(19) United States
(12) Patent Application Publication
(10) Pub. No.: US 2004/0068095 A1
(43) Pub. Date: Apr. 8, 2004

(54) Novel human proteins, polynucleotides encoding them and methods of using the same

(76) Inventors: Richard A. Shimkets, Guilford, CT (US); Raymond J. Taupier JR., East Haven, CT (US); Catherine E. Burgess, Wethersfield, CT (US); Bryan D. Zerhusen, Branford, CT (US); Peter S. Mezes, Old Lyme, CT (US); Luca Rastelli, Guilford, CT (US); Uriel M. Malyankar, Branford, CT (US); William M. Grosse, Branford, CT (US); John P. Alsobrook II, Madison, CT (US); Denise M. Lepley, Branford, CT (US); Kimberly Ann Spytek, New Haven, CT (US); Li Li, Branford, CT (US); Shlomit Edinger, New Haven, CT (US); Valerie Gerlach, Branford, CT (US); Karen Ellerman, Branford, CT (US); John R. MacDougall, Hamden, CT (US); Erik Gunther, Branford, CT (US); Isabelle Millet, Milford, CT (US); David J. Stone, Guilford, CT (US); Glennda Smithson, Guilford, CT (US); Edward S. Szekeres JR., Branford, CT (US); Weizhen Ji, Branford, CT (US)

Correspondence Address: Ivor R. Elrifi, Ph.D., Esq., Mintz, Levin, Cohn, Ferris,, Glovsky and Popeo, P.C., One Financial Center, Boston, MA, 02111, US

(21) Appl. No.: 10/096,625

(22) Filed: Mar. 13, 2002

Related U.S. Application Data
(63) Continuation-in-part of application No. 09/972,211, filed on Oct. 5, 2001.
(60) Provisional application No. 60/275,892, filed on Mar. 14, 2001.
(60) Provisional application No. 60/296,860, filed on Jun. 8, 2001.

(51) Int. Cl. C07K 1/00; C07K 14/00; C07K 17/00

(52) U.S. Cl. 530/350

(57) ABSTRACT

Disclosed herein are nucleic acid sequences that encode novel polypeptides. Also disclosed are polypeptides encoded by these nucleic acid sequences, and antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving any one of these novel human nucleic acids and proteins.

### RELATED APPLICATIONS

This application is a continuation-in-part of U.S. Ser. No. 09/972,211, filed Oct. 5, 2001; and claims priority to U.S. Ser. No. 60/275,892 filed Mar. 14, 2001, and U.S. Ser. No. 60/296,860, filed Jun. 8, 2001 each of which is incorporated by reference in its entirety.

### FIELD OF THE INVENTION

The invention relates to polynucleotides and the polypeptides encoded by such polynucleotides, as well as vectors, host cells, antibodies and recombinant methods for producing the polypeptides and polynucleotides, as well as methods for using the same.

### BACKGROUND OF THE INVENTION

The present invention is based in part on nucleic acids encoding proteins that are new members of the following protein families: alpha-2-macroglobulin, secreted proteins related to angiogenesis, leucine rich-like, cathepsin-L precursor-like, fatty acid-binding protein-like neurolysin precursor-like, gamma-aminobutyric acid (GABA) transporter-like, integrin alpha-7 precursor-like, TMS-2, UNC5 receptor-like, hepatocyte growth factor-like and 26S protease regulatory subunit-like. More particularly, the invention relates to nucleic acids encoding novel polypeptides, as well as vectors, host cells, antibodies, and recombinant methods for producing these nucleic acids and polypeptides.

The alpha-2-macroglobulin (A2M) fatty acid family of proteins are large glycoproteins found in the plasma of vertebrates, in the hemolymph of some invertebrates and in reptilian and avian egg white. A2M-like proteins are able to inhibit all four classes of proteins by a “trapping” mechanism. The A2M-like proteins have a peptide stretch, called the “bait region”, which contains specific cleavage sites for different proteinases. When a proteinase cleaves the bait region, a conformational change is induced in the protein, thus trapping the proteinase. The entrapped enzyme remains active against low molecular weight substrates, whilst its activity toward larger substrates is greatly reduced, due to steric hindrance. Following cleavage in the bait region, a thiol ester bond, formed between the side chains of a cysteine and a glutamine, is cleaved and mediates the covalent binding of the A2M-like protein to the proteinase. A2M is also found in association with senile plaques in Alzheimer's disease. A2M has been implicated biochemically in binding and degradation of amyloid beta protein which accumulates in senile plaques.

The leucine rich-like proteins generally comprise leucine-rich repeats (LRRs), relatively short motifs (22-28 residues in length) found in a variety of cytoplasmic, membrane and extracellular proteins. Although theses proteins are associated with widely different functions, a common property involves protein-protein interaction. Although little is known about the 3-D structure of LRRs, it is believed that they can form amphipathic structures with hydrophilic surfaces capable of acting with membranes. In vitro studies of a synthetic LRR from Drosophila Toll protein have indicated that the peptides form gels by adopting beta-sheet structures that form extended filaments. These results are consistent with the idea that LRRs mediate protein-protein interactions and cellular adhesion. Other functions of LRR-containing proteins include, for example, binding to enzymes and vascular repair. The 3-D structure of ribonuclease inhibitor, a protein containing 15 LRRs, has been determined, revealing LRRs to be a new class of alpha/beta fold. LRRs form elongated non-globular structures and are often flanked by cysteine-rich domains.

Cathepsins are lysosomal proteases that are distributed in many normal tissues and are primarily responsible for intracellular catabolism and turnover. Cathepsin has also been suggested to have roles in the terminal differentiation. Increased levels of cathepsins in tumors together with their ability to degrade extracellular matrix proteins has led to the hypothesis that they are involved in the process of invasion and metastasis. Cathepsin-L is a lysosomal cysteine proteinase belonging to the papain family. This proteinase is different from other members of the mammalian papain family cysteine proteinase in the following ways: (i) the cathepsin-L gene is activated by a variety of growth factors and activated oncogenes, (ii) procathepsin-L, a precursor form of cathepsin L is secreted from various cells, (iii) the mRNA level of cathepsin-L is related to the in vivo metastatic potential of the transformed cells. Thus, the regulation of the cathepsin-L gene and the extracellular functions of secreted procathepsin-L are tightly coupled. Cathepsin-L is induced in tumors by malignant transformation, growth factors, and tumor promoters suggesting they play an important role in tumor invasion and metastasis; additionally, cathepsin-L may be involved in bone resorption implicating possible roles in bone diseases such as osteoporosis, or bone cancers

Fatty acid metabolism in mammalian cells depends on a flux of fatty acids, between the plasma membrane and mitochondria or peroxisomes for beta-oxidation, and between other cellular organelles for lipid synthesis. The fatty acid-binding protein family consists of small, cystolic proteins believed to be involved in the uptake, transport, and solubilization of their hydrophobic ligands. Members of the fatty acid-binding family have highly conserved sequences and tertiary structure. Fatty acid-binding proteins (FABP) were first isolated in the intestine (FABP2) and later found in the liver (FABP1), striated muscle (FABP3), adipocytes (FABP4) and epithelial tissues (E-FABP).

A number of neuropeptidases share two unusual properties: they are strict oligopeptidases—that is they hydrolyze only short peptides—and they cleave at a limited set of sites that are nonetheless diverse in sequence. One neuropeptidase that exemplifies these properties is neurolysin (EC3.4.24.16), a zinc metaloendopeptidase that functions as a monomer of molecular mass 78 kDa (Checler, F. et al., Methods Enzymol. 248 (1995) 593-614; Barrett, A. J. et al., Methods Enzymol. 248 (1995). In vitro, neurolysin cleaves a number of bioactive peptides at sequences that vary widely, and its longest known substrate is only 17 residues in length. The enzyme belongs to the M3 family of metallopeptidases (Rawlings, N. D. et al., Methods Enzymol. 248 (1995) 183-228) along with eight other known peptidases that share extensive sequence homology, including the closely related (60% sequence identity) thimet oligopeptidase (EC3.4.24.15). Enzymes in the M3 family share with several other metallopeptidase families a common active site sequence motif, His-Glu-Xaa-Xaa-His (HEXXH), that forms part of the binding site for the metal cofactor (Matthews, B. W. et al., J. Biol. Chem. 249 (1974) 8030-8044). The two histidines of the motif coordinate the zinc ion, and the glutamate orients and polarizes a water molecule that is believed to act as the attacking nucleophile. Neurolysin is widely distributed in mammalian tissues (Checler, F. et al., Methods Enzymol. 248 (1995) 593-614) and is found in different subcellular locations that vary with cell type. Much of the enzyme is cytosolic, but it also can be secreted or associated with the plasma membrane (Vincent, B. et al., J. Neurosci. 16 (1996) 5049-5059), and some of the enzyme is made with a mitochondrial targeting sequence by initiation at an alternative transcription start site (Kato, A. et al., J. Biol. Chem. 272 (1997) 15313-15322). Although neurolysin cleaves a number of neuropeptides in vitro, its most established (Vincent, B. et al., Brit. J. Pharmacol. 115 (1995) 1053-1063; Barelli, H. et al., Brit. J. Pharmacol. 112 (1994) 127-132; Chabry, J. et al., J. Neurosci. 10 (1990) 3916-3921) role in vivo (along with thimet oligopeptidase) is in metabolism of neurotensin, a 13-residue neuropeptide. It hydrolyzes this peptide between residues 10 and 11, creating shorter fragments that are believed to be inactive. Neurotensin (pGlu-Leu-Tyr-Gln-Asn-Lys-Pro-Arg-Arg-Pro Tyr-Ile-Leu) is found in a variety of peripheral and central tissues where it is involved in a number of effects, including modulation of central dopaminergic and cholinergic circuits, thermoregulation, intestinal motility, and blood pressure regulation (Goedert, M., Trends Neurosci. 7 (1984) 3-5). Neurotensin is also one of the most potent antinocioceptive substances known (Clineschmidt, B. V. et al., Eur. J. Pharmacol. 46 (1977) 395-396), and an inhibitor of neurolysin has been shown to produce neurotensin-induced analgesia in mice (Vincent, B. et al., Br. J. Pharmacol. 121 (1997) 705-710).

Proteins belonging to the famma-aminobutyric acid (GABA) transporter family of proteins play an important role in signal transduction of different cell type such as neuronal and muscle cells. This protein is the human ortholog of VGAT (vesicular GABA transporter) from Rattus norvegicus and unc-47 from C. elegans which are involved in packaging GABA in synaptic vesicles. This protein has a domain similar to the amino acid permease domain found in integral membrane proteins that regulate transport of amino acids. GABA is the product of a biochemical decarboxylation reaction of glutamic acid by the vitamin pyridoxal. GABA serves as a inhibitory neurotransmitter to block the transmission of an impulse from one cell to another in the central nervous system. Medically, GABA has been used to treat both epilepsy and hypertension where it is thought to induce tranquility in individuals who have a high activity of manic behavior and acute agitation.

The integrins are a family of heterodimeric membrane glycoproteins that mediate a wide spectrum of cell-cell and cell-matrix interactions. Their capacity to participate in cellular adhesive processes underlies a wide range of functions. The integrins have preeminent roles in cell migration and morphologic development, differentiation, and metastasis. To a large extent, the diversity and specificity of functions mediated by integrins rest in the structural diversity of the 16 different alpha and 8 beta chains that have been identified and in their ligand-binding and signal transduction capacity. One structural difference in the alpha chains appears to divide them into 2 subgroups. The 1-integrin alpha chains have an insertion of about 180 amino acids in the extracellular region, and the non-I-integrins do not. The functional significance of the I-domain is not known. Alternate splicing increases the structural diversity in the cytoplasmic domains of several integrin alpha and beta chains, and this presumably further expands their functional repertoire. Expression of the alpha-7 integrin gene (ITGA7) is developmentally regulated during the formation of skeletal muscle. Increased levels of expression and production of isoforms containing different cytoplasmic and extracellular domains accompany myogenesis.

A family of genes encoding membrane proteins with a unique structure has been identified in DNA and cDNA clones of various eukaryotes ranging from yeast to human. The nucleotide sequences of three novel cDNAs from Drosophila melanogaster and mouse were determined. The amino acid sequences of the two mouse proteins have human homologs. The gene (TMS-1) encoding the yeast member of this family was disrupted, and the resulting mutant showed no significant phenotype under several stress conditions. The expression of the mouse genes TMS-1 and TMS-2 was examined by in situ hybridization of sections from brain, liver, kidney, heart and testis of an adult mouse as well as in a 1-day-old whole mouse. While the expression of TMS-2 was found to be restricted to the central nervous system, TMS-1 was also expressed in kidney and testis. The expression of TMS-1 and TMS-2 in the brain overlapped and was localized to areas associated with glutamatergic excitatory neurons, such as the hippocampus and cerebral cortex. High-magnification analysis indicated that both mRNAs are expressed in neurons. Semiquantitative analysis of mRNA expression was performed in various parts of the brain. The conservation, unique structure and localization in the mammalian brain of this novel protein family suggest an important biological role.

The vertebrate UNC5 genes, like their Caenorhabditis elegans counterpart, define a family of putative netrin receptors. The netrins comprise a small phylogenetically conserved family of guidance cues important for guiding particular axonal growth cones to their targets. Migration of neurons from proliferative zones to their functional sites is fundamental to the normal development of the central nervous system. Mice homozygous for the spontaneous rostral cerebellar malformation mutation (rcm(s)) or a newly identified transgenic insertion allele (rcm(tg)) exhibit cerebellar and midbrain defects, apparently as a result of abnormal neuronal migration. Laminar structure abnormalities in lateral regions of the rostral cerebellar cortex have been described in homozygous rcm(s) mice. It has been demonstrated that the cerebellum of both rcm(s) and rcm(tg) homozygotes is smaller and has fewer folia than in the wild-type, ectopic cerebellar cells are present in midbrain regions by three days after birth, and there are abnormalities in postnatal cerebellar neuronal migration. The rcm complementary DNA which encodes a transmembrane receptor of the immunoglobulin superfamily has been cloned. The sequence of the rcm protein (Rcm) is highly similar to that of UNC-5, a Caenorhabditis elegans protein that is essential for dorsal guidance of pioneer axons and for the movement of cells away from the netrin ligand, which is encoded by the unc-6 gene. As Rcm is a member of a newly described family of vertebrate homologues of UNC-5 which are netrin-binding proteins, our results indicate that UNC-5-like proteins may have a conserved function in mediating netrin-guided migration (PMID: 9126743, UI: 97271898).

Hepatocyte Growth Factor (HGF), also known as Scatter Factor, is a polypeptide that shows structural homology with enzymes of the blood coagulation cascade. It is a biologically inactive single chain precursor that is then cleaved by specific serine proteases to a fully active alphabeta heterodimer. All the biological responses induced by HGF/SF are elicited by binding to its receptor, a transmembrane tyrosine kinase encoded by the MET proto-oncogene. The signaling cascade triggered by HGF begins with the autophosphorylation of the receptor and is mediated by concomitant activation of different cytoplasmic effectors that bind to the same multifunctional docking site. During development, HGF function is essential: knock-out mice for both ligand and receptor show an embryonic lethal phenotype. HGF/SF displays a unique feature in inducing “branching morphogenesis”, a complex program of proliferation and motogenesis in a number of different cell types. Moreover, HGF is involved in the invasive behavior of several tumor cells both in vivo and in vitro. The role of HGF as a putative therapeutic agent in pathologies characterized by massive cell loss or deregulated cell proliferation is under investigation (PMID: 10641789, UI: 20104755). Additionally, there is increasing evidence that indicates that HGF acts as a multifunctional cytokine on different cell types (PMID: 10760078, UI: 20223576).

The 26S proteosome is the major non-lysosomal protease in eukaryotic cells. This multimeric enzyme is the integral component of the ubiquitin-mediated substrate degradation pathway. It consists of two subcomplexes, the 20S proteosome, which forms the proteolytic core, and the 19S regulator (or PA700), which confers ATP dependency and ubiquitinated substrate specificity on the enzyme. Recent biochemical and genetic studies have revealed many of the interactions between the 17 regulatory subunits, yielding an approximation of the 19S complex topology. Inspection of interactions of regulatory subunits with non-subunit proteins reveals patterns that suggest these interactions play a role in 26S proteosome regulation and localization (PMID: 10664589).

### SUMMARY OF THE INVENTION

The invention is based in part upon the discovery of nucleic acid sequences encoding novel polypeptides. The novel nucleic acids and polypeptides are referred to herein as NOVX, or NOV1, NOV2, NOV3, NOV4, NOV5, NOV6, NOV7, NOV8, NOV9, NOV10, NOV11 and NOV12 nucleic acids and polypeptides. These nucleic acids and polypeptides, as well as derivatives, homologs, analogs and fragments thereof, will hereinafter be collectively designated as “NOVX” nucleic acid or polypeptide sequences.

In one aspect, the invention provides an isolated NOVX nucleic acid molecule encoding a NOVX polypeptide that includes a nucleic acid sequence that has identity to the nucleic acids disclosed in SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199. In some embodiments, the NOVX nucleic acid molecule will hybridize under stringent conditions to a nucleic acid sequence complementary to a nucleic acid molecule that includes a protein-coding sequence of a NOVX nucleic acid sequence. The invention also includes an isolated nucleic acid that encodes a NOVX polypeptide, or a fragment, homolog, analog or derivative thereof. For example, the nucleic acid can encode a polypeptide at least 80% identical to a polypeptide comprising the amino acid sequences of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64 and 200. The nucleic acid can be, for example, a genomic DNA fragment or a cDNA molecule that includes the nucleic acid sequence of any of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199.

Also included in the invention is an oligonucleotide, e.g., an oligonucleotide which includes at least 6 contiguous nucleotides of a NOVX nucleic acid (e.g., SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199) or a complement of said oligonucleotide. Also included in the invention are substantially purified NOVX polypeptides (SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64 and 200). In certain embodiments, the NOVX polypeptides include an amino acid sequence that is substantially identical to the amino acid sequence of a human NOVX polypeptide.

The invention also features antibodies that immunoselectively bind to NOVX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a NOVX nucleic acid, a NOVX polypeptide, or an antibody specific for a NOVX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a NOVX nucleic acid, under conditions allowing for expression of the NOVX polypeptide encoded by the DNA. If desired, the NOVX polypeptide can then be recovered.

In another aspect, the invention includes a method of detecting the presence of a NOVX polypeptide in a sample. In the method, a sample is contacted with a compound that selectively binds to the polypeptide under conditions allowing for formation of a complex between the polypeptide and the compound. The complex is detected, if present, thereby identifying the NOVX polypeptide within the sample.

The invention also includes methods to identify specific cell or tissue types based on their expression of a NOVX.

Also included in the invention is a method of detecting the presence of a NOVX nucleic acid molecule in a sample by contacting the sample with a NOVX nucleic acid probe or primer, and detecting whether the nucleic acid probe or primer bound to a NOVX nucleic acid molecule in the sample.

In a further aspect, the invention provides a method for modulating the activity of a NOVX polypeptide by contacting a cell sample that includes the NOVX polypeptide with a compound that binds to the NOVX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., Cancer, Leukodystrophies, Breast cancer, Ovarian cancer, Prostate cancer, Uterine cancer, Hodgkin disease, Adenocarcinoma, Adrenoleukodystrophy, Cystitis, incontinence, Von Hippel-Lindau (VHL) syndrome, hypercalceimia, Endometriosis, Hirschsprung's disease, Crohn's Disease, Appendicitis, Cirrhosis, Liver failure, Wolfram Syndrome, Smith-Lemli-Opitz syndrome, Retinitis pigmentosa, Leigh syndrome; Congenital Adrenal Hyperplasia, Xerostomia; tooth decay and other dental problems; Inflammatory bowel disease, Diverticular disease, fertility, Infertility, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, Hemophilia, Hypercoagulation, Idiopathic thrombocytopenic purpura, obesity, Diabetes Insipidus and Mellitus with Optic Atrophy and Deafness, Pancreatitis, Metabolic Dysregulation, transplantation recovery, Autoimmune disease, Systemic lupus erythematosus, asthma, arthritis, psoriasis, Emphysema, Scleroderma, allergy, ARDS, Immunodeficiencies, Graft versus host, Alzheimer's disease, Stroke, Parkinson's disease, Huntington's disease, Cerebral palsy, Epilepsy, Multiple sclerosis, Ataxia-telangiectasia, Behavioral disorders, Addiction, Anxiety, Pain, Neurodegeneration, Muscular dystrophy, Lesch-Nyhan syndrome, Myasthenia gravis, schizophrenia, and other dopamine-dysfunctional states, levodopa-induced dyskinesias, alcoholism, pileptic seizures and other neurological disorders, mental depression, Cerebellar ataxia, pure; Episodic ataxia, type 2; Hemiplegic migraine, Spinocerebellar ataxia-6, Tuberous sclerosis, Renal artery stenosis, Interstitial nephritis, Glomerulonephritis, Polycystic kidney disease, Renal tubular acidosis, IgA nephropathy, and/or other pathologies and disorders of the like.

The therapeutic can be, e.g., a NOVX nucleic acid, a NOVX polypeptide, or a NOVX-specific antibody, or biologically active derivatives or fragments thereof.

For example, the compositions of the present invention will have efficacy for treatment of patients suffering from the diseases and disorders disclosed above and/or other pathologies and disorders of the like. The polypeptides can be used as immunogens to produce antibodies specific for the invention, and as vaccines. They can also be used to screen for potential agonist and antagonist compounds. For example, a cDNA encoding NOVX may be useful in gene therapy, and NOVX may be useful when administered to a subject in need thereof. By way of non-limiting example, the compositions of the present invention will have efficacy for treatment of patients suffering from the diseases and disorders disclosed above and/or other pathologies and disorders of the like.

The invention further includes a method for screening for a modulator of disorders or syndromes including, e.g., the diseases and disorders disclosed above and/or other pathologies and disorders of the like. The method includes contacting a test compound with a NOVX polypeptide and determining if the test compound binds to said NOVX polypeptide. Binding of the test compound to the NOVX polypeptide indicates the test compound is a modulator of activity, or of latency or predisposition to the aforementioned disorders or syndromes.

Also within the scope of the invention is a method for screening for a modulator of activity, or of latency or predisposition to disorders or syndromes including, e.g., the diseases and disorders disclosed above and/or other pathologies and disorders of the like by administering a test compound to a test animal at increased risk for the aforementioned disorders or syndromes. The test animal expresses a recombinant polypeptide encoded by a NOVX nucleic acid. Expression or activity of NOVX polypeptide is then measured in the test animal, as is expression or activity of the protein in a control animal which recombinantly-expresses NOVX polypeptide and is not at increased risk for the disorder or syndrome. Next, the expression of NOVX polypeptide in both the test animal and the control animal is compared. A change in the activity of NOVX polypeptide in the test animal relative to the control animal indicates the test compound is a modulator of latency of the disorder or syndrome.

In yet another aspect, the invention includes a method for determining the presence of or predisposition to a disease associated with altered levels of a NOVX polypeptide, a NOVX nucleic acid, or both, in a subject (e.g., a human subject). The method includes measuring the amount of the NOVX polypeptide in a test sample from the subject and comparing the amount of the polypeptide in the test sample to the amount of the NOVX polypeptide present in a control sample. An alteration in the level of the NOVX polypeptide in the test sample as compared to the control sample indicates the presence of or predisposition to a disease in the subject. Preferably, the predisposition includes, e.g., the diseases and disorders disclosed above and/or other pathologies and disorders of the like. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a NOVX polypeptide, a NOVX nucleic acid, or a NOVX-specific antibody to a subject (e.g., a human subject), in an amount sufficient to alleviate or prevent the pathological condition. In preferred embodiments, the disorder, includes, e.g., the diseases and disorders disclosed above and/or other pathologies and disorders of the like.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

NOVX nucleic acids and polypeptides are further useful in the generation of antibodies that bind immuno-specifically to the novel NOVX substances for use in therapeutic or diagnostic methods. These NOVX antibodies may be generated according to methods known in the art, using prediction from hydrophobicity charts, as described in the “Anti-NOVX Antibodies” section below. The disclosed NOVX proteins have multiple hydrophilic regions, each of which can be used as an immunogen. These NOVX proteins can be used in assay systems for functional analysis of various human disorders, which will help in understanding of pathology of the disease and development of new drug targets for various disorders.

The NOVX nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences and their encoded polypeptides. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A. Sequences and Corresponding SEQ ID Numbers

TABLE A
Sequences and Corresponding SEQ ID Numbers

| | | SEQ ID | | |
|---|---|---|---|---|
| | | NO | | |
| NOVX | | (nucleic | SEQ ID NO | |
| Assignment | Internal Identification | acid) | (polypeptide) | Homology |
| 1a | SC_78316254_A | 1 | 2 | Alpha-2-Macroglobulin |
| 1b | CG55051-03 | 199 | 200 | Alpha-2-Macroglobulin |
| 2 | AC005799_A | 3 | 4 | Secreted Proteins Related to |
| | | | | Angiogenesis |
| 3 | SC124141642_A | 5 | 6 | Leucine Rich-like |
| 4 | GMba39917_A/ | 7 | 8 | Cathepsin-L Precursor-like |
| 5 | GMba38118_A | 9 | 10 | Fatty Acid-Binding Protein-like |
| 6a | SC133790496_A | 11 | 12 | Neurolysin Precursor-like |
| 6b | 13375342 | 13 | 14 | Neurolysin Precursor-like |
| 6c | c99.456 | 15 | 16 | Neurolysin Precursor-like |
| 6d | c99.457 | 17 | 18 | Neurolysin Precursor-like |
| 6e | c99.458 | 19 | 20 | Neurolysin Precursor-like |
| 6f | 13375341 | 21 | 22 | Neurolysin Precursor-like |
| 6g | c99.459 | 23 | 24 | Neurolysin Precursor-like |
| 6h | c99.460 | 25 | 26 | Neurolysin Precursor-like |
| 6i | c99.752 | 27 | 28 | Neurolysin Precursor-like |
| 7a | ba122o1 | 29 | 30 | gamma-aminobutyric acid (GABA) |
| | | | | transporter-like |
| 7b | 13374575 | 31 | 32 | gamma-aminobutyric acid (GABA) |
| | | | | transporter-like |
| 7c | 13374576 | 33 | 34 | gamma-aminobutyric acid (GABA) |
| | | | | transporter-like |
| 7d | 13374577 | 35 | 36 | gamma-aminobutyric acid (GABA) |
| | | | | transporter-like |
| 7e | 13374578 | 37 | 38 | gamma-aminobutyric acid (GABA) |
| | | | | transporter-like |
| 7f | 13374579 | 39 | 40 | gamma-aminobutyric acid (GABA) |
| | | | | transporter-like |
| 8a | AC073487_da1 | 41 | 42 | Integrin Alpha 7 Precursor-like |
| 8b | CG53926-02 | 43 | 44 | Integrin Alpha 7 Like-like |
| 9a | 124141642_EXT_da1 | 45 | 46 | TMS-2 |
| 9b | 13375406 | 47 | 48 | TMS-2 |
| 9c | 13375405 | 49 | 50 | TMS-2 |
| 9d | 13375404 | 51 | 52 | TMS-2 |
| 9e | 13375403 | 53 | 54 | TMS-2 |
| 10 | SC121209524_A | 55 | 56 | UNC5 Receptor-like |
| 11a | GMba446g13_A | 57 | 58 | HEPATOCYTE GROWTH |
| | | | | FACTOR-like |
| 11b | cg34a.348 | 59 | 60 | HEPATOCYTE GROWTH |
| | | | | FACTOR-like |
| 11c | cg34a.349 | 61 | 62 | HEPATOCYTE GROWTH |
| | | | | FACTOR-like |
| 12 | GMAC023940_A | 63 | 64 | 26S protease regulatory |
| | | | | subunit-like |

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

NOV1 is homologous to a Alpha-2-Macroglobin-like family of proteins. Thus, the NOV1 nucleic acids, polypeptides, antibodies and related compounds according to the invention will be useful in therapeutic and diagnostic applications implicated in, for example; Alzheimer's disease, inflammation, asthma, allergy and psoriasis, emphysema, pulmonary disease, immune disorders, neurological disorders, and/or other pathologies/disorders.

NOV2 is homologous to the secreted protein related to angiogenesis family of proteins. Thus NOV2 nucleic acids, polypeptides, antibodies and related compounds according to the invention will be useful in therapeutic and diagnostic applications implicated in, for example; abnormal angiogenesis, such as cancer and more specifically, aggressive, metastatic cancer, including tumors of the lungs, kidneys, brain, liver and breasts and/or other pathologies/disorders.

NOV3 is homologous to a family of Leucine rich-like proteins. Thus, the NOV3 nucleic acids and polypeptides, antibodies and related compounds according to the invention will be useful in therapeutic and diagnostic applications implicated in, for example: Lymphatic Diseases, Skin and Connective Tissue Diseases, Diabetes and Kidney Disease, Cancers, tumors, and Brain Disorders, disorders that can be addressed by controlling and directing cell migration, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, Inflammatory bowel disease, Diverticular disease, Crohn's Disease and/or other pathologies/disorders.

NOV4 is homologous to the Cathepsin-L precursor-like family of proteins. Thus, NOV4 nucleic acids, polypeptides, antibodies and related compounds according to the invention will be useful in therapeutic and diagnostic applications implicated in, for example: growth of soft tissue sarcomas; malignant transformation, tumor invasion and metastasis, bone diseases such as osteoporosis, or bone cancers, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Transplantation, Adrenoleukodystrophy, Congenital Adrenal Hyperplasia, Diabetes, Von Hippel-Lindau (VHL) syndrome, Pancreatitis, Endometriosis, Fertility, Inflammatory bowel disease, Diverticular disease, Hirschsprung's disease, Crohn's Disease, Hemophilia, Hypercoagulation, Idiopathic thrombocytopenic purpura, Immunodeficiencies, Osteoporosis, Hypercalceimia, Arthritis, Ankylosin spondylitis, Scoliosis, Endocrine dysfunctions, Diabetes, Growth and reproductive disorders, Psoriasis, Actinic keratosis, Acne, Hair growth, allopecia, pigmentation disorders, endocrine disorders and/or other pathologies/disorders.

NOV5 is homologous to the fatty acid-binding protein family. Thus NOV5 nucleic acids, polypeptides, antibodies and related compounds according to the invention will be useful in therapeutic and diagnostic applications implicated in, for example: psoriasis, basal and squamous cell carcinomas, obesity, diabetes, and/or other pathologies and disorders involving fatty acid transport of skin, oral mucosa as well as other organs, Cardiomyopathy, Atherosclerosis, Hypertension, Congenital heart defects, Aortic stenosis , Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Transplantation, Adrenoleukodystrophy, Congenital Adrenal Hyperplasia, Diabetes, Von Hippel-Lindau (VHL) syndrome, Pancreatitis, Endometriosis, Fertility, Inflammatory bowel disease, Diverticular disease, Hirschsprung's disease, Crohn's Disease, Hemophilia, hypercoagulation, Idiopathic thrombocytopenic purpura, Immunodeficiencies, Osteoporosis, Hypercalceimia, Arthritis, Ankylosing spondylitis, Scoliosis, Endocrine dysfunctions, Diabetes, Growth and reproductive disorders, Psoriasis, Actinic keratosis, Acne, Hair growth, allopecia, pigmentation disorders, endocrine disorders and/or other pathologies/disorders.

NOV6 is homologous to the Neurolysin -like family of proteins. Thus NOV6 nucleic acids, polypeptides, antibodies and related compounds according to the invention will be useful in therapeutic and diagnostic applications implicated in, for example: behavioral neurodegenerative and neuropsychiatric disorders such as schizophrenia, anxiety disorders, bipolar disorders, depression, eating disorders, personality disorders, or sleeping disorders, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Transplantation, Adrenoleukodystrophy, Congenital Adrenal Hyperplasia, Diabetes, Von Hippel-Lindau (VHL) syndrome, Pancreatitis, Endometriosis, Fertility, Inflammatory bowel disease, Diverticular disease, Hirschsprung's disease, Crohn's Disease, Hemophilia, hypercoagulation, Idiopathic thrombocytopenic purpura, Immunodeficiencies, Osteoporosis, Hypercalceimia, Arthritis, Ankylosing spondylitis, Scoliosis, Endocrine dysfunctions, Diabetes, Growth and reproductive disorders, Psoriasis, Actinic keratosis, Acne, Hair growth, allopecia, pigmentation disorders, endocrine disorders and/or other pathologies/disorders.

NOV7 is homologous to members of the PV-1-like family of proteins. Thus, the NOV7 nucleic acids, polypeptides, antibodies and related compounds according to the invention will be useful in therapeutic and diagnostic applications implicated in, for example; cancer, trauma, regeneration (in vitro and in vivo), viral/bacterial/parasitic infections, fertility, neurological disorders and/or other pathologies/disorders.

NOV8 is homologous to the Integrin alpha 7 precursor-like family of proteins. Thus, NOV8 nucleic acids and polypeptides, antibodies and related compounds according to the invention will be useful in therapeutic and diagnostic applications implicated in, for example; Eosinophilic myeloproliferative disorder, Pseudohypoaldosteronism, type IIC, Pseudohypoaldosteronism typeI, Spastic paraplegia-10, Hemolytic anemia due to triosephosphate isomerase deficiency, Imunodeficiency with hyper-IgM, type 2, Clr/Cls deficiency, combined, Cls deficiency, isolated, Leukemia, acute lymphoblastic, Periodic fever, familial, Hypertension, Episodic ataxia/myokymia syndrome, Immunodeficiency with hyper-IgM, type 2, Muscular dystrophy, Lesch-Nyhan syndrome, Myasthenia gravis and other muscular and cellular adhesion disorders and/or other pathologies/disorders.

NOV9 is homologous to members of the TMS-2-like family of proteins. Thus, the NOV9 nucleic acids, polypeptides, antibodies and related compounds according to the invention will be useful in therapeutic and diagnostic applications implicated in, for example; Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, Endocrine dysfunctions, Diabetes, obesity, Growth and Reproductive disorders, Multiple sclerosis, Leukodystrophies, Pain, Neuroprotection, transporter disorders and/or other pathologies/disorders.

NOV10 is homologous to members of the UNC5 receptor-like family of proteins. Thus, the NOV10 nucleic acids, polypeptides, antibodies and related compounds according to the invention will be useful in therapeutic and diagnostic applications implicated in, for example; inflammatory and infectious diseases such as AIDS, cancer therapy, Neurologic diseases, Brain and/or autoimmune disorders like encephalomyelitis, neurodegenerative disorders, Alzheimer's Disease, Parkinson's Disorder, immune disorders, and hematopoietic disorders, endocrine diseases, muscle disorders, inflammation and wound repair, bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome and/or other pathologies/disorders.

NOV11 is homologous to members of the hepatocyte growth factor-like family of proteins. Thus, the NOV11 nucleic acids, polypeptides, antibodies and related compounds according to the invention will be useful in therapeutic and diagnostic applications implicated in, for example; various diseases involving blood coagulation, and hepatocellular carcinoma; cancers including but not limited to lung, breast and ovarian cancer; tumor suppression, senescence, growth regulation, modulation of apoptosis, reproductive control and associated disorders of reproduction, endometrial hyperplasia and adenocarcinoma, psychotic and neurological disorders, Alzheimer's disease, endocrine disorders, inflammatory disorders, gastro-intestinal disorders and disorders of the respiratory system; hematopoiesis, immunotherapy, immunodeficiency diseases, all inflammatory diseases; cancer therapy; autoimmune diseases; obesity, modulation of myofibroblast development; applications to modulation of wound healing; potential applications to control of angiogenesis muscle disorders, neurologic diseases and/or other pathologies/disorders.

NOV12 is homologous to members of the 26S protease regulatory subunit-like family of proteins. Thus, the NOV12 nucleic acids, polypeptides, antibodies and related compounds according to the invention will be useful in therapeutic and diagnostic applications implicated in, for example; eye/lens disorders including but not limited to cataract and Aphakia, Alzheimer's disease, neurodegenerative disorders, inflammation and modulation of the immune response, viral pathogenesis, aging-related disorders, neurologic disorders, cancer and/or other pathologies/disorders.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit, e.g., neurogenesis, cell differentiation, cell proliferation, hematopoiesis, wound healing and angiogenesis.

Additional utilities for the NOVX nucleic acids and polypeptides according to the invention are disclosed herein.

NOV1

NOV 1 includes two novel alpha-2-macroglobulin precursor-like proteins disclosed below. The disclosed proteins have been named NOV1a and NOV1b.

NOV1a

A disclosed NOV1a nucleic acid of 4488 nucleotides (also referred to as SC_{—}78316254_A) encoding a novel alpha-2-macroglobulin precursor-like protein is shown in Table 1A. An open reading frame was identified beginning with an ATG initiation codon at nucleotides 1-3 and ending with a TGA codon at nucleotides 4477-4479. A putative untranslated region downstream from the termination codon is underlined in Table 1A. The start and stop codons are in bold letters.

TABLE 1A
NOV1a Nucleotide Sequence.

(SEQ ID NO:1)

ATGTGGGCTCAGCTCCTTCTAGGAATGTTGGCCCTATCACCACCCATTGCAGAAGAACTTCCAAACTACCTGGTGACATTA
CCAGCCCGGCTAAATTTCCCCTCCGTTCAGAAGGTTTGTTTGGACCTGAGCCCTGGGTACAGTGATGTTAAATTCACGGTT
ACTCTGGAGACCAAGGACAAGACCCAGAAGTTGCTAGAATACTCTGGACTGAAGAAGAGGCACTTACATTGTATCTCCTTT
CTTGTACCACCTCCTGCTGGTGGCACAGAAGAAGTGGCCACAATCCCGGTGTCGGGAGTTGGAAATAACATCAGCTTTGAG
GAGAAGAAAAAGGTTCTAATTCAGAGGCAGGGGAACGGCACCTTTGTACAGACTGACAAACCTCTCTACACCCCAGGGCAG
CAAGTGTATTTCCGCATTGTCACCATGGATAGCAACTTCGTTCCAGTGAATGACAAGTACTCCATGGTGGAACTACAGGAT
CCAAATAGCAACAGGATTGCACAGTGGCTGGAAGTGGTACCTGAGCAAGGCATTGTAGACCTGTCCTTCCAACTGGCACCA
GAGGCAATGCTGGGCACCTACACTGTGGCAGTGGCTGAGGGCAAGACCTTTGGTACTTTCAGTGTGGAGGAATATGTGCTT
TCTCCATTTCTCCTTTTACTCTCTTCAGTGCTGCCGAAGTTTAAGGTGGAAGTGGTGGAACCCAAGGAGTTATCAACGGTG
CAGGAATCTTTCTTAGTAAAAATTTGTTGTAGGTACACCTATGGAAAGCCCATGCTAGGGGCAGTGCAGGTATCTGTGTGT
CAGAAGGCAAATACTTACTGGTATCGAGAGGTGGAACGGGAACAGCTTCCTGACAAATGCAGGAACCTCTCTGGACAGACT
GACAAAACAGGATGTTTCTCAGCACCTGTGGACATGGCCACCTTTGACCTCATTGGATATGCGTACAGCCATCAAATCAAT
ATTGTGGCTACTGTTGTGGAGGAAGGGACAGGTGTGGAGGCCAATGCCACTCAGAATATCTACATTTCTCCACAAATGGGA
TCAATGACCTTTGAAGACACCAGCAATTTTTACCATCCAAATTTCCCCTTCAGTGGGAAGATGCTGCTCAAGTTTCCGCAA
GGCGGTGTGCTCCCTTGCAAGAACCATCTAGTGTTTCTGGTGATTTATGGCACAAATGGAACCTTCAACCAGACCCTGGTT
ACTGATAACAATGGCCTAGCTCCCTTTACCTTGGAGACATCCGGTTGGAATGGGACAGACGTTTCTCTGGAGGGAAAGTTT
CAAATGGAAGACTTAGTATATAATCCGGAACAAGTGCCACGTTACTACCAAAATGCCTACCTGCACCTGCGACCCTTCTAC
AGCACAACCCGCAGCTTCCTTGGCATCCACCGGCTAAACGGCCCCTTGAAATGTGGCCAGCCCCAGGAAGTGCTGGTGGAT
TATTACATCGACCCGGCCGATGCAAGCCCTGACCAAGAGATCAGCTTCTCCTACTATTTAATAGGGAAAGGAAGTTTGGTG
ATGGAGGGGCAGAAACACCTGAACTCTAAGAAGAAAGGACTGAAAGCCTCCTTCTCTCTCTCACTGACCTTCACTTCGAGA
CTGGCCCCTGATCCTTCCCTGGTGATCTATGCCATTTTTCCCAGTGGAGGTGTTGTAGCTGACAAAATTCAGTTCTCAGTC
GAGATGTGCTTTGACAATCAGCAGCTTCCAGGAGCAGAAGTGGAGCTGCAGCTGCAGGCAGCTCCCGGATCCCTGTGTGCG
CTCCGGGCGGTGGATGAGAGTGTCTTACTGCTTAGGCCAGACAGAGAGCTGAGCAACCGCTCTGTCTATGGGATGTTTCCA
TTCTGGTATGGTCACTACCCCTATCAAGTGGCTGAGTATGATCAGTGTCCAGTGTCTGGCCCATGGGACTTTCCTCAGCCC
CTCATTGACCCAATGCCCCAAGGGCATTCGAGCCAGCGTTCCATTATCTGGAGGCCCTCGTTCTCTGAAGGCACGGACCTT
TTCAGCTTTTTCCGGGACGTGGGCCTGAAAATACTGTCCAATGCCAAAATCAAGAAGCCACTAGATTGCAGTCACAGATCT
CCAGAATACAGCACTGCTATGGGTGGCGGTGGTCATCCAGAGGCTTTTGAGTCATCAACTCCTTTACATCAAGCAGAGGAT
TCTCAGGTCCGCCAGTACTTCCCAGAGACCTGGCTCTGGGATCTGTTTCCTATTGGTAACTCGGGGAAGGAGGCGGTCCAC
GTCACAGTTCCTGACGCCATCACCGAGTGGAAGGCGATGAGTTTCTGCACTTCCCAGTCAAGAGGCTTCGGGCTTTCACCC
ACTGTTGGACTAACTGCTTTCAAGCCGTTCTTTGTTGACCTGACTCTCCCTTACTCAGTAGTCCGTGGGGAATCCTTTCGT
CTTACTGCCACCATCTTCAATTACCTAAAGGATTGCATCAGGGTTCAGACTGACCTGGCTAAATCGCATGAGTACCAGCTA
GAATCATGGGCAGATTCTCAGACCTCCAGTTGTCTCTGTGCTGATGACGCAAAAACCCACCACTGGAACATCACAGCTGTC
AAATTGGGTCACATTAACTTTACTATTAGTACAAAGATTCTGGACAGCAATGAACCATGTGGGGGCCAGAAGGGGTTTGTT
CCCCAAAAGGGCCGAAGTGACACGCTCATCAAGCCAGTTCTCGTCAAACCTGAGGGAGTCCTGGTGGAGAAGACACACAGC
TCATTGCTGTGCCCAAAAGGAGGAAAGGTGGCATCTGAATCTGTCTCCCTGGAGCTCCCAGTGGACATTGTTCCTGACTCG
ACCAAGGCTTATGTTACGGTTCTGGGAGACATTATGGGCACAGCCCTGCAGAACCTGGATGGTCTGGTGCAGATGCCCAGT
GGCTGTGGCGAGCAGAACATGGTCTTGTTTGCTCCCATCATCTATGTCTTGCAGTACCTGGAGAAGGCAGGGCTGCTGACG
GAGGAGATCAGGTCTCGGGCAGTGGGTTTCCTGGAAATAGGGTACCAGAAGGAGCTGATGTACAAACACAGCAATGGCTCA
TACAGTGCCTTTGGGGAGCGAGATGCAAATGGAAACACATGGCTGACAGCGTTTGTCACAAAATGCTTTGGCCAAGCTCAG
AAATTCATCTTCATTGATCCCAAGAACATCCAGGATGCTCTCAAGTGGATGGCAGGAAACCAGCTCCCCAGTGGCTGCTAT
GCCAACGTGGGAAATCTCCTTCACACAGCTATGAAGGGTGGTGTTGATGATGAGGTCTCCTTGACTGCGTATGTCACAGCT
GCATTGCTGGAGATGGGAAAGGATGTAGATGACCCAATGGTGAGTCAGGGTCTACGGTGTCTCAAGAATTCGGCCACCTCC
ACGACCAACCTCTACACACAGGCCCTGTTGGCTTACATTTTCTCCCTGGCTGGGGAAATGGACATCAGAAACATTCTCCTT
AAACAGTTAGATCAACAGGCTATCATCTCAGGAGAATCCATTTACTGGAGCCAGAAACCTACTCCATCATCGAACGCCAGC
CCTTGGTCTGAGCCTGCGGCTGTAGATGTGGAACTCACAGCATATGCATTGTTGGCCCAGCTTACCAAGCCCAGCCTGACT
CAAAAGGAGATAGCGAAGGCCACTAGCATAGTGGCTTGGTTGGCCAAGCAACACAATGCATATGGGGGCTTCTCTTCTACT
CAGGATACTGTAGTTGCTCTCCAAGCTCTTGCCAAATATGCCACTACCGCCTACATGCCATCTGAGGAGATCAACCTGGTT
GTAAAATCCACTGAGAATTTCCAGCGCACATTCAACATACAGTCAGTTAACAGATTGGTATTTCAGCAGGATACCCTGCCC
AATGTCCCTGGAATGTACACGTTGGAGGCCTCAGGCCAGGGCTGTGTCTATGTGCAGACGGTGTTGAGATACAATATTCTC
CCTCCCACAAATATGAAGACCTTTAGTCTTAGTGTGGAAATAGGAAAAGCTAGATGTGAGCAACCGACTTCACCTCGATCC
TTGACTCTCACTATTCACACCAGTTATGTGGGGAGCCGTAGCTCTTCCAATATGGCTATTGTGGAAGTGAAGATGCTATCT
GGGTTCAGTCCCATGGAGGGCACCAATCAGTTACTTCTCCAGCAACCCCTGGTGAACAAGGTTGAATTTGGAACTGACACA
CTTAACATTTACTTGGATGAGCTCATTAAGAACACTCAGACTTACACCTTCACCATCAGCCAAAGTGTGCTGGTCACCAAC
TTGAAACCAGCAACCATCAAGGTCTATGACTACTACCTACCAGGTTCTTTTAAATTATCTCAGTACACAATTGTGTGGTCC
ATGAACAATGACAGCATAGTGGACTCTGTGGCACGGCACCCAGAACCACCCCCTTTCAAGACAGAAGCATTTATTCCTTCA
CTTCCTGGGAGTGTTAACAACTGATAGCTACCA

In a search of public sequence databases, the NOV1a nucleic acid sequence has 840 of 1324 bases (63%) identical to a Rattus norvegicus alpha-2-macroglobulin precursor mRNA (GENBANK-ID: Rat A2M) (E=1.3e⁻¹¹⁹). Public nucleotide databases include all Genbank databases and the GeneSeq patent database.

In all BLAST alignments herein, the “E-value” or “Expect” value is a numeric indication of the probability that the aligned sequences could have achieved their similarity to the BLAST query sequence by chance alone, within the database that was searched. For example, the probability that the subject (“Sbjct”) retrieved from the NOV1 a BLAST analysis, e.g., Rattus norvegicus alpha-2-macroglobulin precursor mRNA, matched the Query NOV1a sequence purely by chance is 1.3e⁻¹¹⁹. The Expect value (E) is a parameter that describes the number of hits one can “expect” to see just by chance when searching a database of a particular size. It decreases exponentially with the Score (S) that is assigned to a match between two sequences. Essentially, the E value describes the random background noise that exists for matches between sequences.

The Expect value is used as a convenient way to create a significance threshold for reporting results. The default value used for blasting is typically set to 0.0001. In BLAST 2.0, the Expect value is also used instead of the P value (probability) to report the significance of matches. For example, an E value of one assigned to a hit can be interpreted as meaning that in a database of the current size one might expect to see one match with a similar score simply by chance. An E value of zero means that one would not expect to see any matches with a similar score simply by chance. See, e.g., http://www.ncbi.nlm.nih.gov/Education/BLASTinfo/. Occasionally, a string of X's or N's will result from a BLAST search. This is a result of automatic filtering of the query for low-complexity sequence that is performed to prevent artifactual hits. The filter substitutes any low-complexity sequence that it finds with the letter “N” in nucleotide sequence (e.g., “NNNNNNNN”) or the letter “X” in protein sequences (e.g., “XXX”). Low-complexity regions can result in high scores that reflect compositional bias rather than significant position-by-position alignment. Wootton and Federhen, Methods Enzymol 266:554-571, 1996.

The disclosed NOV1a polypeptide (SEQ ID NO:2) encoded by SEQ ID NO:1 has 1492 amino acid residues and is presented in Table 1B using the one-letter amino acid code. Signal P, Psort and/or Hydropathy results predict that NOV1 a has a signal peptide and is likely to be localized outside the cell with a certainty of 0.3703. The most likely cleavage site for a NOV1a peptide is between amino acids 17 and 18, at: AIA-EE.

TABLE 1B
Encoded NOV1a Protein Sequence.

(SEQ ID NO:2)

MWAQLLLGMLALSPAIAEELPNYLVTLPARLNFPSVQKVCLDLSPGYSDVKFTVTLETKDKTQKLLEYSGLK
KRHLHCISFLVPPPAGGTEEVATIRVSGVGNNISFEEKKKVLIQRQGNGTFVQTDKPLYTPGQQVYFRIVTM
DSNFVPVNDKYSMVELQDPNSNRIAQWLEVVPEQGIVDLSFQLAPEAMLGTYTVAVAEGKTFGTFSVEEYVL
SPFLLLLSSVLPKFKVEVVEPKELSTVQESFLVKICCRYTYGKPMLGAVQVSVCQKANTYWYREVEREQLPD
KCRNLSGQTDKTGCFSAPVDMATFDLIGYAYSHQINIVATVVEEGTGVEANATQNIYISPQMGSMTFEDTSN
FYHPNFPFSGKMLLKFPQGGVLPCKNHLVFLVIYGTNGTFNQTLVTDNNGLAPFTLETSGWNGTDVSLEGKF
QMEDLVYNPEQVPRYYQNAYLHLRPFYSTTRSFLGIHRLNGPLKCGQPQEVLVDYYIDPADASPDQEISFSY
YLIGKGSLVMEGQKHLNSKKKGLKASFSLSLTFTSRLAPDPSLVIYAIFPSGGVVADKIQFSVEMCFDNQQL
PGAEVELQLQAAPGSLCALRAVDESVLLLRPDRELSNRSVYGMFPFWYGHYPYQVAEYDQCPVSGPWDFPQP
LIDPMPQGHSSQRSIIWRPSFSEGTDLFSFFRDVGLKILSNAKIKKPVDCSHRSPEYSTAMGGGGHPEAFES
STPLHQAEDSQVRQYFPETWLWDLFPIGNSGKEAVHVTVPDAITEWKAMSFCTSQSRGFGLSPTVGLTAFKP
FFVDLTLPYSVVRGESFRLTATIFNYLKDCIRVQTDLAKSHEYQLESWADSQTSSCLCADDAKTHHWNITAV
KLGHINFTISTKILDSNEPCGGQKGFVPQKGRSDTLIKPVLVKPEGVLVEKTHSSLLCPKGGKVASESVSLE
LPVDIVPDSTKAYVTVLGDIMGTALQNLDGLVQMPSGCGEQNMVLFAPIILVLQYLEKAGLLTEEIRSRAVG
FLEIGYQKELMYKHSNGSYSAFGERDGNGNTWLTAFVTKCFGQAQKFIFIDPKNIQDALKWMAGNQLPSGCY
ANVGNLLHTAMKGGVDDEVSLTAYVTAALLEMGKDVDDPMVSQGLRCLKNSATSTTNLYTQALLAYIFSLAG
EMDIRNILLKQLDQQAIISGESIYWSQKPTPSSNASPWSEPAAVDVELTAYALLAQLTKPSLTQKEIAKATS
IVAWLAKQHNAYGGFSSTQDTVVALQALAKYATTAYMPSEEINLVVKSTENFQRTFNIQSVNRLVFQQDTLP
NVPGMYTLEASGQGCVYVQTVLRYNILPPTNMKTFSLSVEIGKARCEQPTSPRSLTLTIHTSYVGSRSSSNM
AIVEVKMLSGFSPMEGTNQLLLQQPLVKKVEFGTDTLNIYLDELIKNTQTYTFTISQSVLVTNLKPATIKVY
DYYLPGSFKLSQYTIVWSMNNDSIVDSVARHPEPPPFKTEAFIPSLPGSVNN

The NOV1a amino acid sequence has 595 of 1450 amino acid residues (41%) identical to, and 873 of 1450 residues (60%) positive with, the Homo sapiens 1474 amino acid residue alpha-2-macroglobulin precursor protein (ptnr: SPTREMBL-ACC:P01023) (E=2.0e⁻²⁷⁹).

NOV1b

A disclosed NOV1b nucleic acid of 4492 nucleotides (also referred to as CG55051-03) encoding a novel alpha-2-macroglobulin precursor-like protein is shown in Table 1C. An open reading frame was identified beginning with an ATG initiation codon at nucleotides 1-3 and ending with a TGA codon at nucleotides 4375-4377. A putative untranslated region downstream from the termination codon is underlined in Table 1C. The start and stop codons are in bold letters.

TABLE 1C
NOV1b Nucleotide Sequence.

(SEQ ID NO:199)

ATGTGGGCTCAGCTCCTTCTAGGAATGTTGGCCCTATCACCAGCCATTGCAGAAGAACTTCCAAACTACCTG
GTGACATTACCAGCCCGGCTAAATTTCCCCTCCGTTCAGAAGGTTTGTTTGGACCTGAGCCCTGGGTACAGT
GATGTTAAATTCACGGTTACTCTGGAGACCAAGGACAAGACCCAGAAGTTGCTAGAATACTCTGGACTGAAG
AAGAGGCACTTACATTGTATCTCCTTTCTTGTACCACCTCCTGCTGGTGGCACAGAAGAAGTGGCCACAATC
CGGGTGTCGGGAGTTGGAAATAACATCAGCTTTGAGGAGAAGAAAAAGGTTCTAATTCAGAGGCAGGGGAAC
GGCACCTTTGTACAGACTGACAAACCTCTCTACACCCCAGGGCAGCAAGTGTATTTCCGCATTGTCACCATG
GATAGCAACTTCGTTCCAGTGAATGACAAGTACTCCATGGTGGAACTACAGGATCCAAATAGCAACAGGATT
GCACAGTGGCTGGAAGTGGTACCTGAGCAAGGCATTGTAGACCTGTCCTTCCAACTGGCACCAGAGGCAATG
CTGGGCACCTACACTGTGGCAGTGGCTGAGGGCAAGACCTTTGGTACTTTCAGTGTGGAGGAATATGTGCTT
TCTCCATTTCTCCTTTTACTCTCTTCAGTGCTGCCGAAGTTTAAGGTGGAAGTGGTGGAACCCAAGGAGTTA
TCAACGGTGCAGGAATCTTTCTTAGTAAAAATTTGTTGTAGGTACACCTATGGAAAGCCCATGCTAGGGGCA
GTGCAGGTATCTGTGTGTCAGAAGGCAAATACTTACTGGTATCGAGAGGTGGAACGGGAACAGCTTCCTGAC
AAATGCAGGAACCTCTCTGGACAGACTGACAAAACAGGATGTTTCTCAGCACCTGTGGACATGGCCACCTTT
GACCTCATTGGATATGCGTACAGCCATCAAATCAATATTGTGGCTACTGTTGTGGAGGAAGGGACAGGTGTG
GAGGCCAATGCCACTCAGAATATCTACATTTCTCCACAAATGGGATCAATGACCTTTGAAGACACCAGCAAT
TTTTACCATCCAAATTTCCCCTTCAGTGGGAAGATGCTGCTCAAGTTTCCGCAAGGCGGTGTGCTCCCTTGC
AAGAACCATCTAGTGTTTCTGGTGATTTATGGCACAAATGGAACCTTCAACCAGACCCTGGTTACTGATAAC
AATGGCCTAGCTCCCTTTACCTTGGAGACATCCGGTTGGAATGGGACAGACGTTTCTCTGGAGGGAAAGTTT
CAAATGGAAGACTTAGTATATAATCCGGAACAAGTGCCACGTTACTACCAAAATGCCTACCTGCACCTGCGA
CCCTTCTACAGCACAACCCGCAGCTTCCTTGGCATCCACCGGCTAAACGGCCCCTTGAAATGTGGCCAGCCC
CAGGAAGTGCTGGTGGATTATTACATCGACCCGGCCGATGCAAGCCCTGACCAAGAGATCAGCTTCTCCTAC
TATTTAATAGGGAAAGGAAGTTTGGTGATGGAGGGGCAGAAACACCTGAACTCTAAGAAGAAAGGACTGAAA
GCCTCCTTCTCTCTCTCACTGACCTTCACTTCGAGACTGGCCCCTGATCCTTCCCTGGTGATCTATGCCATT
TTTCCCAGTGGAGGTGTTGTAGCTGACAAAATTCAGTTCTCAGTCGAGATGTGCTTTGACAATCAGCAGCTT
CCAGGAGCAGAAGTGGACCTGCAGCTGCAGGCAGCTCCCGGATCCCTGTGTGCGCTCCGGGCGGTGGATGAG
AGTGTCTTACTGCTTAGGCCAGACAGAGAGCTGAGCAACCGCTCTGTCTATGGGATGTTTCCATTCTGGTAT
GGTCACTACCCCTATCAAGTGGCTGAGTATGATCAGTGTCCAGTGTCTGGCCCATGGGACTTTCCTCAGCCC
CTCATTGACCCAATGCCCCAAGGGCATTCGAGCCAGCGTTCCATTATCTGGAGGCCCTCGTTCTCTGAAGGC
ACGGACCTTTTCAGCTTTTTCCGGGACGTGGGCCTGAAAATACTGTCCAATGCCAAAATCAAGAAGCCAGTA
GATTGCAGTCACAGATCTCCAGAATACAGCACTGCTATGGGTGGCGGTGGTCATCCAGAGGCTTTTGAGTCA
TCAACTCCTTTACATCAAGCAGAGGATTCTCAGGTCCGCCAGTACTTCCCAGAGACCTGGCTCTGGGATCTG
TTTCCTATTGGTAACTCGGGGAAGGAGGCGGTCCACGTCACAGTTCCTGACGCCATCACCGAGTGGAAGGCG
ATGAGTTTCTGCACTTCCCAGTCAAGAGGCTTCGGGCTTTCACCCACTGTTGGACTAACTGCTTTCAAGCCG
TTCTTTGTTGACCTGACTCTCCCTTACTCAGTAGTCCGTGGGGAATCCTTTCGTCTTACTGCCACCATCTTC
AATTACCTAAAGGATTGCATCAGGGTTCAGACTGACCTGGCTAAATCGCATGAGTACCAGCTAGAATCATGG
GCAGATTCTCAGACCTCCAGTTGTCTCTGTGCTGATGACGCAAAAACCCACCACTGGAACATCACAGCTGTC
AAATTGGGTCACATTAACTTTACTATTAGTACAAAGATTCTGGACAGCAATGAACCATGTGGGGGCCAGAAG
GGGTTTGTTCCCCAAAAGGGCCGAAGTGACACGCTCATCAAGCCAGTTCTCGTCAAACCTGAGGGAGTCCTG
GTGGAGAAGACACACAGCTCATTGCTGTGCCCAAAAGGAGGAAAGGTGGCATCTGAATCTGTCTCCCTGGAG
CTCCCAGTGGACATTGTTCCTGACTCGACCAAGGCTTATGTTACGGTTCTGGGAGACATTATGGGCACAGCC
CTGCAGAACCTGGATGGTCTGGTGCAGATGCCCAGTGGCTGTGGCGAGCAGAACATGGTCTTGTTTGCTCCC
ATCATCTATGTCTTGCAGTACCTGGAGAAGGCAGGGCTGCTGACGGAGGAGATCAGGTCTCGGGCAGTGGGT
TTCCTGGAAATAGGGTACCAGAAGGAGCTGATGTACAAACACAGCAATGGCTCATACAGTGCCTTTGGGGAG
CGAGATGGAAATGGAAACACATGGCTGACAGCGTTTGTCACAAAATGCTTTGGCCAAGCTCAGAAATTCATC
TTCATTGATCCCAAGAACATCCAGGATGCTCTCAAGTGGATGGCAGGAAACCAGCTCCCCAGTGGCTGCTAT
GCCAACGTGGGAAATCTCCTTCACACAGCTATGAAGGGTGGTGTTGATGATGAGGTCTCCTTGACTGCGTAT
GTCACAGCTGCATTGCTGGAGATGGGAAAGGATGTAGATGACCCAATGGTGAGTCAGGGTCTACGGTGTCTC
AAGAATTCGGCCACCTCCACGACCAACCTCTACACACAGGCCCTGTTGGCTTACATTTTCTCCCTGGCTGGG
GAAATGGACATCAGAAACATTCTCCTTAAACAGTTAGATCAACAGGCTATCATCTCAGGAGAATCCATTTAC
TGGAGCCAGAAACCTACTCCATCATCGAACGCCAGCCCTTGGTCTGAGCCTGCGGCTGTAGATGTGGAACTC
ACAGCATATGCATTGTTGGCCCAGCTTACCAAGCCCAGCCTGACTCAAAAGGAGATAGCGAAGGCCACTAGC
ATAGTGGCTTGGTTGGCCAAGCAACACAATGCATATGGGGGCTTCTCTTCTACTCAGGATACTGTAGTTGCT
CTCCAAGCTCTTGCCAAATATGCCACTACCGCCTACATGCCATCTGAGGAGATCAACCTGGTTGTAAAATCC
ACTGAGAATTTCCAGCGCACATTCAACATACAGTCAGTTAACAGATTGGTATTTCAGCAGGATACCCTGCCC
AATGTCCCTGGAATGTACACGTTGGAGGCCTCAGGCCAGGGCTGTGTCTATGTGCAGACGGTGTTGAGATAC
AATATTCTCCCTCCCACAAATATGAAGACCTTTAGTCTTAGTGTGGAAATAGGAAAAGCTAGATGTGAGCAG
CCGACTTCACCTCGATCCTTGACTCTCACTATTCACACCAGTTATGTGGGGAGCCGTAGCTCTTCCAATATG
GCTATTGTGGAAGTGAAGATGCTATCTGGGTTCAGTCCCATGGAGGGCACCAATCAGTTACTTCTCCAGCAA
CCCCTGGTGAAGAAGGTTGAATTTGGAACTGACACACTTAACATTTACTTGGATGAGCTCATTAAGAACACT
CAGACTTACACCTTCACCATCAGCCAAAGTGTGCTGGTCACCAACTTGAAACCAGCAACCATCAAGGTCTAT
GACTACTACCTACCAGATGAACAGGCAACAATTCAGTATTCTGATCCCTGTGAATGAGGATAGGAGCTGGAA
ACTCAATTAGTCCTCTGTGACATTTACTGGAGGGTGGAACATTCTTCTGTCGCTTGAAGCAGAACTCATTCA
ATCAAATAATTTAATTTCTCTGACTAGT

The NOV1b nucleic acid is localized to chromosome 12 and has 840 of 1324 bases (63%) identical to a gb:GENBANK-ID:RATA2M|acc:J02635.1 mRNA from Rattus norvegicus (Rat liver alpha-2-macroglobulin mRNA, complete cds) (E=1.8e⁻¹¹⁹)

The disclosed NOV1b polypeptide (SEQ ID NO:200) encoded by SEQ ID NO: 199 has 1458 amino acid residues and is presented in Table 1D using the one-letter amino acid code. Signal P, Psort and/or Hydropathy results predict that NOV1b has a signal peptide and is likely to be localized extracellularly with a certainty of 0.3703. The most likely cleavage site for a NOV1b peptide is between amino acids 17 and 18, at: AIA-EE.

TABLE 1D
Encoded NOV1b Protein Sequence.

(SEQ ID NO:200)

MWAQLLLGMLALSPAIAEELPNYLVTLPARLNFPSVQKVCLDLSPGYSDVKFTVTLETKDKTQKLLEYSGLK
KRHLHCISFLVPPPAGGTEEVATIRVSGVGNNISFEEKKKVLIQRQGNGTFVQTDKPLYTPGQQVYFRIVTM
DSNFVPVNDKYSMVELQDPNSNRIAQWLEVVPEQGIVDLSFQLAPEAMLGTYTVAVAEGKTFGTFSVEEYVL
SPFLLLLSSVLPKFKVEVVEPKELSTVQESFLVKICCRYTYGKPMLGAVQVSVCQKANTYWYREVEREQLPD
KCRNLSGQTDKTGCFSAPVDMATFDLIGYAYSHQINIVATVVEEGTGVEANATQNIYISPQMGSMTFEDTSN
FYHPNFPFSGKMLLKFPQGGVLPCKNHLVFLVIYGTNGTFNQTLVTDNNGLAPFTLETSGWNGTDVSLEGKF
QMEDLVYNPEQVPRYYQNAYLHLRPFYSTTRSFLGIHRLNGPLKCGQPQEVLVDYYIDPADASPDQEISFSY
YLIGKGSLVMEGQKHLNSKKKGLKASFSLSLTFTSRLAPDPSLVIYAIFPSGGVVADKIQFSVEMCFDNQQL
PGAEVELQLQAAPGSLCALRAVDESVLLLRPDRELSNRSVYGMFPFWYGHYPYQVAEYDQCPVSGPWDFPQP
LIDPMPQGHSSQRSIIWRPSFSEGTDLFSFFRDVGLKILSNAKIKKPVDCSHRSPEYSTAMGGGGHPEAFES
STPLHQAEDSQVRQYFPETWLWDLFPIGNSGKEAVHVTVPDAITEWKAMSFCTSQSRGFGLSPTVGLTAFKP
FFVDLTLPYSVVRGESFRLTATIFNYLKDCIRVQTDLAKSHEYQLESWADSQTSSCLCADDAKTHHWNITAV
KLGHINFTISTKILDSNEPCGGQKGFVPQKGRSDTLIKPVLVKPEGVLVEKTHSSLLCPKGGKVASESVSLE
LPVDIVPDSTKAYVTVLGDIMGTALQNLDGLVQMPSGCGEQNMVLFAPIIYVLQYLEKAGLLTEEIRSRAVG
FLEIGYQKELMYKESNGSYSAFGERDGNGNTWLTAFVTKCFGQAQKFIFIDPKNIQDALKWMAGNQLPSGCY
ANVGNLLHTAMKGGVDDEVSLTAYVTAALLEMGKDVDDPMVSQGLRCLKNSATSTTNLYTQALLAYIFSLAG
EMDIRNILLKQLDQQAIISGESIYWSQKPTPSSNASPWSEPAAVDVELTAYALLAQLTKPSLTQKEIAKATS
IVAWLAKQHNAYGGFSSTQDTVVALQALAKYATTAYMPSEEINLVVKSTENFQRTFNIQSVNRLVFQQDTLP
NVPGMYTLEASGQGCVYVQTVLRYNILPPTNMKTFSLSVEIGKARCEQPTSPRSLTLTIHTSYVGSRSSSNM
AIVEVKMLSGFSPMEGTNQLLLQQPLVKKVEFGTDTLNIYLDELIKNTQTYTFTISQSVLVTNLKPATIKVY
DYYLPDEQATIQYSDPCE

The NOV1b amino acid sequence has 599 of 1454 amino acid residues (41%) identical to, and 876 of 1454 amino acid residues (60%) similar to, the 1474 amino acid residue ptnr:SWISSNEW-ACC:P01023 protein from Homo sapiens (Human) (Alpha-2-macroglobulin Precursor (Alpha-2-M)) (E=6.0e⁻²⁸²).

NOV1b is expressed in at least the following tissues: brain, vulva, bronchus, skin and pool mammalian tissues. Expression information was derived from the tissue sources of the included in the derivation of the invention.

The NOV1a and 1b proteins are very closely homologous as shown in the alignment in

[image]

[image]

Homologies to either of the above NOV1 proteins will be shared by the other NOV1 protein insofar as they are homologous to each other as shown above. Any reference to NOV1 is assumed to refer to both of the NOV1 proteins in general, unless otherwise noted.

The disclosed NOV1a polypeptide has homology to the amino acid sequences shown in the BLASTP data listed in Table 1F.

TABLE 1F
BLAST results for NOV1a

| Gene Index/ | | Length | Identity | Positives | |
|---|---|---|---|---|---|
| Identifier | Protein/Organism | (aa) | (%) | (%) | Expect |
| gi\|14765710\|ref\|XP_{—} | alpha 2 | 1474 | 593/1486 | 870/1486 | 0.0 |
| 006925.4\| | macroglobulin | | (39%) | (57%) | |
| | precursor | | | | |
| | [Homo sapiens] | | | | |
| gi\|4557225\|ref\|NP_{—} | alpha 2 | 1474 | 591/1486 | 869/1486 | 0.0 |
| 000005.1\| | macroglobulin | | (39%) | (57%) | |
| | precursor | | | | |
| | [Homo sapiens] | | | | |
| gi\|224053\|prf\|\| | macroglobulin | 1450 | 585/1471 | 861/1471 | 0.0 |
| 1009174A | alpha2 | | (39%) | (57%) | |
| | [Homo sapiens] | | | | |
| gi\|6978425\|ref\|NP_{—} | alpha-2- | 1472 | 578/1483 | 867/1483 | 0.0 |
| 036620.1\| | macroglobulin | | (38%) | (57%) | |
| | [Rattus norvegicus] | | | | |
| gi\|2144118\|pir\|\| | alpha- | 1476 | 570/1495 | 858/1495 | 0 |
| JC5143 | macroglobulin | | (38%) | (57%) | |
| | precursor | | | | |
| | [Cavia porcellus] | | | | |

The homology between these and other sequences is shown graphically in the ClustalW analysis shown in Table 1G. In the ClustalW alignment of the NOV1a protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

[image]

[image]

The presence of identifiable domains in NOV1a, as well as all other NOVX proteins, was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results for NOV1a, as disclosed in Tables 1H and 1I, were collected from the Conserved Domain Database (CDD) with Reverse Position Specific BLAST analyses. This BLAST analysis software samples domains found in the Smart and Pfam collections. For Tables 1H, 1I and all successive DOMAIN sequence alignments, fully conserved single residues are indicated by black shading or by the sign (|) and “strong” semi-conserved residues are indicated by grey shading or by the sign (+). The “strong” group of conserved amino acid residues may be any one of the following groups of amino acids: STA, NEQK, NHQK, NDEQ, QHRK, MILV, MILF, HY, FYW.

Tables 1H and 1I lists the domain description from DOMAIN analysis results against NOV1a. This indicates that the NOV1a sequence has properties similar to those of other proteins known to contain these domains.

TABLE 111
Domain Analysis of NOV1a

gnl|Pfm|pfam00207, A2M, Alpha-2-macroglobulin family. This family
includes the C-terminal region of the alpha-2-macroglobulin family.
(SEQ ID NO:70)
Length=751 residues, 98.5% aligned
Score=563 bits (1451), Expect=2e−161

| | | | |
|---|---|---|---|
| NOV1a | 728 | EDSQVRQYFPETWLWDLFPIGNSGKEAVHVTVPDAITEWKAMSFCTSQSRGFGLSPTGL | 787 |
| | | +\| +\| \|\|\|\|+\|\|\|++ + \| \|++\|+\|\|+\|\| \|+ ++ \| ++\| ++ \| \| | |
| Pfam00207 | 4 | DDITIRSYFPESWLWEVEEVDRSPVLTVNITLPDSITTWEILAVSLSNTKGLCVADPVEL | 63 |
| NOV1a | 788 | TAFKPFFVDLTLPYSVVRGESFRLTATIFNYL-KDCIRVQTDLAKSHEYQLESWADSQTS | 846 |
| | | \| \|+ \|\|++\| \|\|\|\|\|\|\|\|\| \| \| ++\|\|\| \|+\| \|\|\| \| | |
| Pfam00207 | 64 | TVFQDFFLELRLPYSVVRGEQVELRAVLYNYLPSQDIKV--------VVQLEVEPLCQAG | 115 |
| NOV1a | 847 | SCLCADDAKTHHWNITAVKLGHINFTISTKILDSNEPCGGQKGFVPQKGRSDTLIKPVLV | 906 |
| | | \| \| ++ \| ++\| + \| \| \|\|+ \| ++\| + \| | |
| Pfam00207 | 116 | FCALATQRTRSSQSVRPKSLSSVSFPVVVVPLASGLSLVEVVASVPEFFVKDAVVKTLKV | 175 |
| NOV1a | 907 | KPEGVLVEKTHSSLLCP---KGGKVASESVSLELPVDIVPD-STKAYVTVLGDIMGTALQ | 962 |
| | | +\|\|\| \|+\| \|\|\|\| \| \|\| + \| \| \|\|\| +\| ++\| \|\| + \|+\| | |
| Pfam00207 | 176 | EPEGARKEETVSSLLLPPEHLGGGLEVSEVPALKLPDDVPDTEAEAVISVQGDPVAQAIQ | 235 |
| NOV1a | 963 | N------LDGLVQMPSGCGEQNMVLFAPIIYVLQYLEKAGLLTE---EIRSRAVGFLEIG | 1013 |
| | | \| \|+ \|+++\|\|\|\|\|\|\|\|\|+ \|\| +\|\|\| \|\|++ + + + +\|+ + \| | |
| Pfam00207 | 236 | NTLSGEGLNNLLRLPSGCGEQNMIYMAPTVYVLHYLDETWQWEKPGTKKKQKAIDLINKG | 295 |
| NOV1a | 1014 | YQKELMYKHSNGSYSAFGERDGNGNTWLTAFVTKCFGQAQKFIFIDPKNIQDALKW-MAG | 1072 |
| | | \|\|++\| \|+ ++\|\|\|+\|\| \| +\|\|\|\|\|\|\| \| \| \|\|+ ++\|\|\| ++\| \|+\|\| + | |
| Pfam00207 | 296 | YQRQLNYRKADGSYAAFLHRA--SSTWLTAFVLKVFSQARNYVFIDEEHICGAVKWLILN | 353 |
| NOV1a | 1073 | NQLPSGCYANVGNLLHTAMKGGVDD----EVSLTAYVTAALLEMGKDVDDPMVSQGLRCL | 1128 |
| | | \| \| + \| ++\| \|\|\|\|\| \| \|\|+\|\|\|++\| \|\|\|\| \|+\|+ \| \| | |
| Pfam00207 | 354 | QQKDDGVFRESGPVIHNEMKGGVGDDAEVEVTLTAFITIALLEAKLVCISPVVANALSIL | 413 |
| NOV1a | 1129 | KNSATSTTN------LYTQALLAYIFSLAGEMDIRNILLKQLDQQAIISGESIYWS--QK | 1180 |
| | | \| \| \| +\|\| \|\| \|\| +\|\|\| + +\|\| \| ++ + +\| \|\| | |
| Pfam00207 | 414 | KASDYLLENYANGQRVYTLALTAYALALAGVLHKLKEILKSLKEELYKALVKGHWERPQK | 473 |
| NOV1a | 1181 | PTPSSNASPWSEPAAVDVELTAYALLAQLTKPSLTQKEIAKATSIVAWLAKQHNAYGGFS | 1240 |
| | | \| + +\| \| \|\|+\|+\|\|\|\|\| \|\| \| ++ \| +\| \|\| +\| \|\|\| | |
| Pfam00207 | 474 | PKDAPGHPYSPQPQAAAVEMTSYALLALLT--LLPFPKVEMAPKVVKWLTEQQYYGGGFG | 531 |
| NOV1a | 1241 | STQDTVVALQALAKYATTAYMPSE-EINLVVKSTEN-FQRTFNIQSVNRLVFQQDTLP-N | 1297 |
| | | \|\|\|\|\|\|+\|\|\|\|\|+\|\| \| +++ ++\| \|+ \| \| + \| + + \|\| \| | |
| Pfam00207 | 532 | STQDTVMALQALSKYGIATPTHKEKNLSVTIQSPSGSFKSHFQILNNNAFLLRPVELPLN | 591 |
| NOV1a | 1298 | VPGMYTLEASGQGCVYVQTVLRYNILPPTNMKTFSLSVEIGKARCEQPTSPR-SLTLTIH | 1356 |
| | | \| + +\|\|\| + + \| \|\| +\| \| \| \| +\| \| +\| + \| \|+\| | |
| Pfam00207 | 592 | EGFTVTAKVTGQGTLTLVTTYRYKVLDKKNTFCFDLKIETVPDTCVEPKGAKNSDYLSIC | 651 |
| NOV1a | 1357 | TSYVGSRSSSNMAIVEVKMLSGFSPMEGT--NQLLLQQPLVKKVEFGTDTLNIYLDELIK | 1414 |
| | | \| \| \|\|\|\| \| \|\|\| ++ \|\|+\|\| \|++ \| \| \| + + +\|\|\|++ | |
| Pfam00207 | 652 | TRYAGSRSDSGMAIADISMLTGFIPLKPDLKKLENGVDRYVSKYEIDGNHVLLYLDKVSH | 711 |
| NOV1a | 1415 | -NTQTYTFTISQSVLVTNLKPATIKVYDYYLP 1445 | |
| | | \|+ \| \| \| \| \|+\|\|++\|\|\|\|\|\| \| | |
| Pfam00207 | 712 | SETECVGFKIHQDFEVGLLQPASVKVYDYYEP 743 | |

TABLE 1I
Domain Analysis of NOV1a

gnl|Pfam|pfam01835, A2M_N, Alpha-2-macroglobulin family N-terminal
region. This family includes the N-terminal region of the alpha-2-macroglobulin
family. (SEQ ID NO:71)
Length=620 residues, 98.4% aligned
Score=236 bits (603), Expect=5e−63

| | | | |
|---|---|---|---|
| NOV1a | 5 | LLLGMLALSPAIAEEL--PNYLVTLPARLNFPSVQKVCLDLSPGYSDVKFTVTLETKDKT | 62 |
| | | \|\| +\| \| \| \| \|+\| +\|+ \| + +\|\|\|+ \| \| \|\|+\| + | |
| Pfam01835 | 2 | LLWLLLLLLLFFDSSLQKPRYMVIVPSILRTETPEKVCVQLHDLNETVTVTVSLHSFPGK | 61 |
| NOV1a | 63 | QKLLEYSGLK---KRHLHCISFLVPPPA---GGTEEVATIRVSGVGNNISFEEKKKVLIQ | 116 |
| | | + \| + \| \|\|+\|\| \|\| \| \| + + \| \| +\|+\|\| \|\|+ | |
| Pfam01835 | 62 | RNLSSLFTVLLSSKDLFHCVSFTVPQPGLFKSSKGEESFVVVQVKGPTHTFKEKVTVLVS | 121 |
| NOV1a | 117 | RQGNGTFVQTDKPLYTPGQQVYFRIVTMDSNFVPVNDKYSMVELQDPNSNRIAQWLEVVP | 176 |
| | | + \|+\|\|\|\|\|+\|\|\|\|\| \| +\|+ ++\| \| \|+\|+ +\| ++\|\| \|\|+ \|\| | |
| Pfam01835 | 122 | SRRGLVFIQTDKPIYTPGQTVRYRVFSVDENLRPLNELI-LVYIEDPEGNRVDQWEVNKL | 180 |
| NOV1a | 177 | EQGIVDLSFQLAPEAMLGTYTVAV---AEGKTFGT--FSVEEYVLSPFLLLLSSVLPKFK | 231 |
| | | \| \|\| \|\|\| + \| + \|\|+ + + ++ \| \| \|+\|\| + | |
| Pfam01835 | 181 | EGGIFQLSFPIPSEPIQGTWKIVARYESGPESNYTHYFEVKEY---------VLPSFEVS | 231 |
| NOV1a | 232 | VEVVEPKELSTVQESFLVKICCRYTYGKPMLGAVQVSVCQKANTYWYREVEREQLPDKCR | 291 |
| | | + +\| + \| \| \|\| \|\|\|\|\|\|\|+ \| \| \| \| +++\| | |
| Pfam01835 | 232 | ITPPKPFIYYDNFKEFEVTICARYTYGKPVPGVAYVRFGVK------DEDGKKELLAGLE | 285 |
| NOV1a | 292 | NLSGQTDKTG--CFSAPVDMATFDLIGYAY-SHQINIVATVVEEGTGVEANA-TQNIYIS | 347 |
| | | + \| \| \| \| \| + \| + + \|+\| \| \| \| \| | |
| Pfam01835 | 286 | ERAKLLDGNGEICLSQEVLLKELQLKNEDLEGKSLYVAVAVIESEGGDMEEAELGGIKIV | 345 |
| NOV1a | 348 | PQMGSMTFEDTSNFYHPNFPFSGKMLLKFPQGGVLPCKNHLVFLVIYGTNGTFNQTLVTD | 407 |
| | | + \| \| + + \| \|\| \|+\|+ \| \| \| \| \| + + ++ \|\| | |
| Pfam01835 | 346 | RSPYKLKFVKTPSHFKPGIPFFLKVLVVDPDGS--PAPNVPVK--VSAQDASYYSNGTTD | 401 |
| NOV1a | 408 | NNGLAPFTLETSGWNGTDVSLEGKFQMEDLVYNPEQVPRYYQNAYLHLRPFYSTTRSFLG | 467 |
| | | +\|\|\| \|++ \|\| + +\|+ + ++\| + \|\| + \| + \| | |
| Pfam01835 | 402 | EDGLAQFSINTS--GISSLSITVRTNHKELPEEVQAHAEAQATAYSTVSL--SKSYIHLS | 457 |
| NOV1a | 468 | IHRLNGPLKCGQPQEVLVDYYIDPADASPDQEISFSYYLIGKGSLVMEGQKHNLSKKKGL | 527 |
| | | \| \| \| \|\| ++ + + + \| \| ++ \|\| +\| \|++ ++ | |
| Pfam01835 | 458 | IER---TLPCGPGVGEQANFILRGKSLGELKILHFYYLIMSKGKIVKTGRE----PREPG | 510 |
| NOV1a | 528 | KASFSLSLTFTSRLAPDPSLVIYAIFPSGGVVADKIQFSVEMCFDN-----------QQL | 576 |
| | | + \|\|\|\|+ \| \|\|\| \|\| \| \| \| \| \|\|\|\| + \|\| \| \| +\| | |
| Pfam01835 | 511 | QGLFSLSIPVTPDLAPSFRLVAYYILPQGEVVADSVWIDVEDCCANKLDLSFSPSKDYRL3 | 570 |
| NOV1a | 577 | PGAEVELQLQAAPGSLCALRAVDESVLLLRPDRELSNRSVY 617 | |
| | | \| +\|+\|+++\| \| \|\| \|\|\|\|\|\|++\| \|\|+\| +\|\| \|\| | |
| Pfam01835 | 571 | PAQQVKLRVEADPQSLVALRAVDQAVYLLKPKAKLSMSKVY 611 | |

The Alpha-2-Macroglobulin (A2M) family of large glycoproteins are responsible for catalyzing the phosphorylation of the light chain of myosin during the contraction of smooth muscle. Thus, the myosin light chain kinase (MLCK) proteins serve as a key enzyme in muscle contraction and have been shown by immunohistology to be present in neurons and glia. The cDNA for human MLCK has been cloned from hippocampus and shown to encode a protein sequence 95% similar to smooth muscle MLCKs but less than 60% similar to skeletal muscle MLCKs. The cDNA clone detected two RNA transcripts in human frontal and entorhinal cortex, in hippocampus, and in jejunum, one corresponding to MLCK and the other probably to telokin, the carboxy-terminal 154 residues of MLCK expressed as an independent protein in smooth muscle. The levels of expression have been shown to be lower in brain than in smooth muscle. The acidic C-terminus of all MLCKs from both brain and smooth muscle resembles the C-terminus of tubulins. By PCR and Southern blotting using 2 somatic cell hybrid panels, the MLCK gene has been localized to 3cen-q21. Since the MLCK disclosed herein is an MLCK, the chromosomal locus has been assigned as Chromosome 3cen-q21.

Phosphorylation of myosin II regulatory light chains (RLC) by Ca2+/calmodulin (CAM)-dependent MLCK is a critical step in the initiation of smooth muscle and non-muscle cell contraction. Post-translational modifications to MLCK down-regulate enzyme activity, suppressing RLC phosphorylation, myosin II activation and tension development.

A2M glycoproteins are found in the plasma of vertebrates, in the hemolymph of some invertebrates and in reptilian and avian egg white (Sottrup-Jensen, J. Biol. Chem. 264: 11539-11542, 1989). A2M-like proteins are able to inhibit all four classes of proteinases by a ‘trapping’ mechanism. They have a peptide stretch, called the ‘bait region’, which contains specific cleavage sites for different proteinases. When a proteinase cleaves the bait region, a conformational change is induced in the protein, thus trapping the proteinase. The entrapped enzyme remains active against low molecular weight substrates, whilst its activity toward larger substrates is greatly reduced, due to steric hindrance. Following cleavage in the bait region, a thiol ester bond, formed between the side chains of a cysteine and a glutamine, is cleaved and mediates the covalent binding of the A2M-like protein to the proteinase.

By the electroimmunoassay of Laurell, Bergqvist and Nilsson (Scand. J. Haemat. 23: 433-436, 1979) found deficient A2M in a 37-year-old man, his mother, and one daughter. A2M is, like alpha-1-antitrypsin, alpha-2-antiplasmin, and antithrombin III, a protease inhibitor. It inhibits many proteases, including trypsin, thrombin and collagenase. The deficient persons were apparently heterozygotes. No clinical disadvantage resulted from the deficiency. Poller et al. (Hum. Genet. 83: 93-96, 1989) detected an alteration in the A2M gene in a patient with serum A2M deficiency and chronic lung disease since childhood. The alteration involved restriction sites detected with 10 different enzymes and was thought to have been caused by major deletion or rearrangement in the gene. Nine of the restriction enzymes used detected no polymorphism in 40 healthy control subjects and 39 patients with chronic obstructive pulmonary disease. The patient was heterozygous for the A2M alteration; Poller et al. (1989) suggested that this was responsible for the pulmonary disease.

Umans et al. (J. Biol. Chem. 270: 19778-19785, 1995) created mice lacking the A2M gene. The knockout mice were not only viable, but more resistant to endotoxin. They produced normal-sized litters and showed no obvious phenotypic abnormalities. Webb et al. (J. Biol. Chem. 271: 24982-24988, 1996) demonstrated that murine alpha-2-macroglobulin binds TGF-beta and inhibits TGF-beta-receptor interactions. They suggested that these results explain the endotoxin-insensitive phenotype of the knockout mice.

A2M, a serum pan-protease inhibitor, has been implicated in Alzheimer disease (AD; 104300) based on its ability to mediate the clearance and degradation of A-beta, the major component of amyloid beta deposits. Blacker et al. (Nature Genet. 19: 357-360, 1998) analyzed a deletion in the A2M gene at the 5-prime splice site of ‘exon II’ of the bait region (exon 18) and found that inheritance of the deletion, designated A2M-2, conferred increased risk for AD (Mantel-Haenzel odds ratio=3.56, P=0.001). The sibship disequilibrium test (SDT) also revealed a significant association between A2M and AD. These values were comparable to those obtained for the APOE4 allele in the same sample, but in contrast to APOE4, A2M-2 did not affect age of onset. The observed association that A2M with AD did not appear to account for the previously published linkage of AD to chromosome 12, which Blacker et al. (1998) were unable to confirm in their sample. Thus, A2M, the A2M receptor (LRP1; 107770), and the genes for 2 other LRP (low density lipoprotein-related protein) ligands, APOE (107741) and APP (104760), had all been genetically linked to AD, suggesting that these proteins may participate in the common neuropathogenic pathway leading to the disease. Liao et al. (Hum. Molec. Genet. 7: 1953-1956, 1998) found an association between the val100-to-ile polymorphism (103950.0001) and AD.

In a study of AD in 3 samples of patients in the U.K., Dow et al. (Nature Genet. 22: 16-17, 1999) failed to show a strong association between A2M*2 and AD risk. Rudrasingham et al. Nature Genet. 22: 17-19, 1999) did not find associations between AD and A2M*2, or genotypes containing A2M*2, in a powerful, case-control sample. Using the same family-based association methods employed by Blacker et al. (1998), Rogaeva et al. (Nature Genet. 22: 19-21, 1999) also could not replicate the association of A2M*2 in 2 comparable, independent familial AD data sets or in a larger data set of families from the same data set tested by Blacker et al. (1998). Furthermore, they were unable to document any biologic effect of the A2M*2 allele on A2M RNA splicing, protein monomeric molecular mass, or protein levels in brain, liver, or plasma of A2M*2 carriers. Rogaeva et al. (1999) concluded that prior genetic evidence for an AD susceptibility locus on chromosome 12 likely arose from genetic variations other than in A2M*2 alleles. In a reply to these criticisms, Blacker et al. (1999) commented that they did not find it surprising that the case-control studies conducted by the groups of Rudrasingham et al. (1999), Rogaeva et al. (1999), and Dow et al. (1999) could not find an association. They suggested that the findings highlighted differences between family-based and case-control association studies. In an accompanying editorial (Anonymous, 1999), the editor of Nature Genetics pointed out the difficulties in evaluating the results of association studies. Unresolved fundamental issues included such matters as the significance threshold of a true association (especially in light of multiple-hypothesis testing aggravated by publication bias for positive associations), how best to analyze a given data set, and what constitutes a valid refutation. The editors suggested that they would expect manuscripts reporting genetic associations to include an estimate of the effect size and to contain either a replication in an independent sample or physiologically meaningful data supporting a functional role of the polymorphism in question.

The above defined information for NOV1 suggests that this A2M precursor-like protein may function as a member of a A2M precursor family. Therefore, the NOV1 nucleic acids and proteins of the invention are useful in potential therapeutic applications implicated in various diseases and disorders described below and/or other pathologies. For example, the NOV1 compositions of the present invention will have efficacy for treatment of patients suffering from adrenoleukodystrophy, Alzheimer's disease, autoimmune disease, allergies, addiction, anxiety, ataxia-telangiectasia, asthma, ARDS, atherosclerosis, behavioral disorders, aortic stenosis, atrial septal defect (ASD), atrioventricular (A-V) canal defect, ductus arteriosus, cerebral palsy, congenital adrenal hyperplasia, cirrhosis, cardiomyopathy, congenital heart defects, diabetes, diverticular disease, epilepsy, emphysema, endometriosis, endocrine dysfunctions, graft versus host disease, glomerulonephritis, growth and reproductive disorders, hemophilia, hypercoagulation, hypercalceimia, Huntington's disease, hypertension, hypogonadism, fertility, idiopathic thrombocytopenic purpura, immunodeficiencies, interstitial nephritis, IgA nephropathy, lymphaedema, inflammatory bowel disease, Lesch-Nyhan syndrome, leukodystrophies, multiple sclerosis, muscular dystrophy, myasthenia gravis, neurodegeneration, neuroprotection, obesity, Parkinson's disease, pain, polycystic kidney disease, pulmonary stenosis, pancreatitis, renal artery stenosis, renal tubular acidosis, stroke, systemic lupus erythematosus, scleroderma, subaortic stenosis, transplantation, tuberous sclerosis, Von Hippel-Lindau (VHL) syndrome, ventricular septal defect (VSD), valve diseases, ulcers, cancer and other diseases and disorders. The NOV1 nucleic acid encoding A2M precursor-like protein, and the A2M precursor-like protein of the invention, or fragments thereof, may further be useful in diagnostic applications, wherein the presence or amount of the nucleic acid or the protein are to be assessed.

NOV2

A disclosed NOV2 nucleic acid of 2021 nucleotides (also referred to as AC005799_A) encoding a novel secreted protein related to angiogenesis is shown in Table 2A. An open reading frame was identified beginning with an ATG initiation codon at nucleotides 40-42 and ending with a TAA codon at nucleotides 1667-1669. Putative untranslated regions upstream from the initiation codon and downstream from the termination codon are underlined in Table 2A. The start and stop codons are in bold letters.

TABLE 2A
NOV2 Nucleotide Sequence.

(SEQ ID NO:3)

TTGATGGTATTAAGGGGTAGGGTCTTTGGGAGGTGTCTCAATGCCGGGGCTGCGCCGGGACCGCCTACTG
ACTCTGCTACTGCTGGGCGCGCTGCTCTCCGCCGACCTCTACTTCCACCTCTGGCCCCAAGTACAGCGCC
AGCTGCGGCCTCGGGAGCGCCCGCGGGGGTGCCCGTGCACCGGCCGCGCCTCCTCCCTGGCGCGGGACTC
GGCCGCAGCTGCCTCGGACCCCGGCACGATCGTGCACAACTTTTCCCGAACCGAGCCCCGGACTGAACCG
GCTGGCGGCAGCCACAGCGGGTCGAGCTCCAAGTTGCAGGCCCTCTTCGCCCACCCGCTGTACAACGTCC
CGGAGGAGCCGCCTCTCCTGGGAGCCGAGGACTCGCTCCTGGCCAGCCAGGAGGCGCTGCGGTATTACCG
GAGGAAGGTGGCCCGCTGGAACAGGCGACACAAGATGTACAGAGAGCAGATGAACCTTACCTCCCTGGAC
CCCCCACTGCAGCTCCGACTCGAGGCCAGCTGGGTCCAGTTCCACCTGGGTATTAACCGCCATGGGCTCT
ACTCCCGGTCCAGCCCTGTTGTCAGCAAACTTCTGCAAGACATGAGGCACTTTCCCACCATCAGTGCTGA
TTACAGTCAAGATGAGAAAGCCTTGCTGGGGGCATGTGACTGCACCCAGATTGTGAAACCCAGTGGGGTC
CACCTCAAGCTGGTGCTGAGGTTCTCGGATTTCGGGAAGGCCATGTTCAAACCCATGAGACAGCAGCGAG
ATGAGGAGACACCAGTGGACTTCTTCTACTTCATTGACTTTCAGAGACACAATGCTGAGATCGCAGCTTT
CCATCTGGACAGGATTCTGGACTTCCGACGGGTGCCGCCAACAGTGGGGAGGATAGTAAATGTCACCAAG
GAAATCCTAGAGGTCACCAAGAATGAAATCCTGCAGAGTGTTTTCTTTGTCTCTCCAGCGAGCAACGTGT
GCTTCTTCGCCAAGTGTCCATACATGTGCAAGACGGAGTATGCTGTCTGTGGCAACCCACACCTGCTGGA
GGGTTCCCTCTCTGCCTTCCTGCCGTCCCTCAACCTGGCCCCCAGGCTGTCTGTGCCCAACCCCTGGATC
CGCTCCTACACACTGGCAGGAAAAGAGGAGTGGGAGGTCAATCCCCTTTACTGTGACACAGTGAAACAGA
TCTACCCGTACAACAACAGCCAGCGGCTCCTCAATGTCATCGACATGGCCATCTTCGACTTCTTGATAGG
GAATATGGACCGGCACCATTATGAGATGTTCACCAAGTTCGGGGATGATGGGTTCCTTATTCACCTTGAC
AACGCCAGAGGGTTCGGACGACACTCCCATGATGAAATCTCCATCCTCTCGCCTCTCTCCCAGTGCTGCA
TGATAAAAAAGAAAACACTTTTGCACCTGCAGCTGCTGGCCCAAGCTGACTACAGACTCAGCGATGTGAT
GCGAGAATCACTGCTGGAAGACCAGCTCAGCCCTGTCCTCACTGAACCCCACCTCCTTGCCCTGGATCGA
AGGCTCCAAACCATCCTAAGGACAGTGGAGGGGTGCATAGTGGCCCATGGACAGCAGAGTGTCATAGTCG
ACGGCCCAGTGGAACAGTCGGCCCCAGACTCTGGCCAGGCTAACTTGACAAGCTAA GGGCTGGCAGAGTC
CAGTTTCAGAAAATACGCCTGGAGCCAGAGCAGTCGACTCGAGTGCCGACCCTGCGTCCTCACTCCCACC
TGTTACTGCTGGGAGTCAAGTCAGCTAGGAAGGAAGCAGGACATTTTCTCAAACAGCAAGTGGGGCCCAT
GGAACTGAATCTTTACTCCTTGGTGCACCGCTTCTGTCGTGCGTTGCCTTGCTCCGTTTTTCCCAAAAAG
CACTGGCTTCATCAAGGCCACCGACGATCTCCTGAGTGCACTGGGAAATCTGGGTATAGGTCAGGCTTGG
CAGCCTTGATCCCAGGAGAGTACTAATGGTAACAAGTCAAATAAAAGGACATCAAGTGGAA

The disclosed NOV2 nucleic acid sequence, localized to chromosome 17, has 1378 of 1378 bases (100%) identical to Homo sapiens HSM801386 mRNA (GENBANK-ID: HSM801386 (E=2.0e⁻³⁰⁵).

A NOV2 polypeptide (SEQ ID NO:4) encoded by SEQ ID NO:3 has 541 amino acid residues and is presented using the one-letter code in Table 2B. Signal P, Psort and/or Hydropathy results predict that NOV2 contains a signal peptide and is likely to be localized outside the cell with a certainty of 0.7045. The most likely cleavage site for a NOV2 peptide is between amino acids 33 and 34, at: VQR-QL.

TABLE 2Bphz,1/44 !Encoded NOV2 Protein Sequence.
(SEQ ID NO:4)

MPGLRRDRLLTLLLLGALLSADLYFHLWPQVQRQLRPRERPRGCPCTGRASSLARDSAAAASDPGTIVHN
FSRTEPRTEPAGGSHSGSSSKLQALFAHPLYNVPEEPPLLGAEDSLLASQEALRYYRRKVARWNRRHKMY
REQMNLTSLDPPLQLRLEASWVQFHLGINRHGLYSRSSPVVSKLLQDMRHFPTISADYSQDEKALLGACD
CTQIVKPSGVHLKLVLRFSDFGKAMFKPMRQQRDEETPVDFFYFIDFQRHNAEIAAFHLDRILDFRRVPP
TVGRIVNVTKEILEVTKNEILQSVFFVSPASNVCFFAKCPYMCKTEYAVCGNPHLLEGSLSAFLPSLNLA
PRLSVPNPWIRSYTLAGKEEWEVNPLYCDTVKQIYPYNNSQRLLNVIDMAIFDFLIGNMDRHHYEMFTKF
GDDGFLIHLDNARGFGRHSHDEISILSPLSQCCMIKKKTLLHLQLLAQADYRLSDVMRESLLEDQLSPVL
TEPHLLALDRRLQTILRTVEGCIVAHGQQSVIVDGPVEQSAPDSGQANLTS

The NOV2 amino acid sequence has 340 of 340 amino acid residues (100%) identical to a Homo sapiens CAB61412 protein (GENBANK-ID:CAB61412) (E=2.9e⁻¹⁸⁴). Essentially, the sequence constitutes a 5′ extension of HSM801386.

Tissue expression data, obtained by Taqman analysis, reveals strong expression by activated endothelial cells, indicating that the NOV2 secreted protein might be involved in the angiogenic process and could be useful to identify and treat angiogenic processes. Analysis also reveals that the NOV2 gene is overexpressed by kidney tumors compared with their normal adjacent tissues and also strongly expressed by liver and liver tumors, Sage analysis also reveals NOV2 expression in ovarian tumors (Tables 21-23).

NOV2 also has homology to the amino acid sequences shown in the BLASTP data listed in Table 2C.

TABLE 2C
BLAST results for NOV2

| Gene Index/ | | Length | Identity | Positives | |
|---|---|---|---|---|---|
| Identifier | Protein/Organism | (aa) | (%) | (%) | Expect |
| gi\|1359998\|pir\|\| | hypothetical | 340 | 340/340 | 340/340 | 0.0 |
| T42684 | protein | | (100%) | (100%) | |
| | DKFZp434F2322.1 | | | | |
| | (fragment) | | | | |
| | [Homo sapiens] | | | | |
| gi\|14776441\|ref\|XP_{—} | hypothetical | 307 | 306/307 | 306/307 | 1e−174 |
| 045783.1 | protein | | (99%) | (99%) | |
| | DKFZp434F2322 | | | | |
| | [Homo sapiens] | | | | |
| gi\|9368881\|emb\| | hypothetical | 311 | 176/286 | 225/286 | 1e−104 |
| CAB99089.1\| | protein | | (61%) | (78%) | |
| (AL390147) | [Homo sapiens] | | | | |
| gi\|13385516\|ref\|NP_{—} | hypothetical | 249 | 132/237 | 180/237 | 3e−76 |
| 085042.1\| | protein | | (55%) | (75%) | |
| | MGC7673 | | | | |
| | [Mus musculus] | | | | |
| gi\|7404833\|pir\| | hypothetical | 512 | 143/381 | 207/381 | 4e−66 |
| T23035 | protein H03A11.1 | | (37%) | (53%) | |
| | [Caenorhabditis | | | | |
| | elegans] | | | | |

The homology of these sequences is shown graphically in the ClustalW analysis shown in Table 2D.

[image]

[image]

[image]

The above defined information for NOV2 suggests that the NOV2 protein may function as a member of a family of novel secreted proteins related to angiogenesis.

Therefore, the NOV2 nucleic acids and proteins of the invention are useful in potential therapeutic applications implicated in various diseases and disorders described below and/or other pathologies. For example, the NOV2 compositions of the present invention will have efficacy for treatment of patients suffering from abnormal angiogenesis, such as cancer and more specifically, aggressive, metastatic cancer, including tumors of the lungs, kidneys, brain, liver and breasts. The NOV2 nucleic acid encoding secreted proteins related to angiogenesis, and the secreted proteins related to angiogenesis of the invention, or fragments thereof, may further be useful in diagnostic applications, wherein the presence or amount of the nucleic acid or the protein are to be assessed.

NOV3

A disclosed NOV3 nucleic acid of 1869 nucleotides (also referred to as SC124141642_A) encoding a novel leucine rich-like protein is shown in Table 3A. An open reading frame was identified beginning with an ATG initiation codon at nucleotides 17-19 and ending with a TGA codon at nucleotides 1841-1843. Putative untranslated regions upstream from the initiation codon and downstream from the termination codon are underlined in Table 3A. The start and stop codons are in bold letters.

TABLE 3A
NOV3 Nucleotide Sequence

(SEQ ID NO:5)

CTCCCCGCCCGCCCGCATGTGCGCAGAGGATGGTGGCGCGGCCCTAGGCCCACGCTCCGCATGACCTGCTGGCTGT
GCGTCCTGAGCCTGCCCCTGCTCCTGCTGCCCGCGGCGCCGCCCCCGGCTGGAGGCTGCCCGGCCCGCTGCGAGTGCACC
GTGCAGACCCGCGCGGTGGCCTGCACGCGCCGCCGCCTGACCGCCGTGCCCGACGGCATCCCGGCCGAGACCCGCCTGCT
GGAGCTCAGCCGCAACCGCATCCGCTGCCTGAACCCGGGCGACCTGGCCGCGCTGCCCGCGCTGGAGGAGCTGGACCTGA
GCGAGAACGCCATCGCGCACGTGGAGCCCGGCGCCTTCGCCAACCTGCCGCGCCTGCGCGTCCTGCGTCTCCGTGGCAAC
CAGCTGAAGCTCATCCCGCCCGGGGTCTTCACGCGCCTGGACAACCTCACGCTGCTGGACCTGAGCGAGAACAAGCTGGT
AATCCTGCTGGACTACACTTTCCAGGACCTGCACAGCCTGCGCCGGCTGGAAGTGGGCGACAACGACCTGGTATTCGTCT
CGCGCCGCGCCTTCGCGGGGCTGCTGGCCCTGGAGGAGCTGACCCTGGAGCGCTGCAACCTCACGGCTCTGTCCGGGGAG
TCGCTGGGCCATCTGCGCAGCCTGGGCGCCCTGCGGCTGCGCCACCTGGCCATCGCCTCCCTGGAGGACCAGAACTTCCG
CAGGCTGCCCGGGCTGCTGCACCTGGAGATTGACAACTGGCCGCTGCTGGACGAGGTGGCGGCGGGCAGCCTGCGGGGCC
TGAACCTGACCTCGCTGTCGGTCACCCACACCAACATCACCGCCGTGCCGGCCGCCGCGCTGCGGCACCAGGCGCACCTC
ACCTGCCTCAATCTGTCGCACAACCCCATCAGCACGGTGCCGCGGGGGTCGTTCCGGGACCTGGTCCGCCTGCGCGAGCT
GCACCTGGCCGGGGCCCTGCTGGCTGTGGTGGAGCCGCAGGCCTTCCTGGGCCTGCGCCAGATCCGCCTGCTCAACCTCT
CCAACAACCTGCTCTCCACGTTGGAGGAGAGCACCTTCCACTCGGTGAACACGCTAGAGACGCTGCGCGTGGACGGGAAC
CCGCTGGCCTGCGACTGTCGCCTGCTGTGGATCGTGCAGCGTCGCAAGACCCTCAACTTCGACGGGCGGCTGCCGGCCTG
CGCCACCCCGGCCGAGGTGCGCGGCGACGCGCTGCGAAACCTGCCGGACTCCGTGCTGTTCGAGTACTTCGTGTGCCGCA
AACCCAAGATCCGGGAGCGGCGGCTGCAGCGCGTCACGGCCACCGCGGGCGAAGACGTCCGCTTCCTCTGCCGCGCCGAG
GGCGAGCCGGCGCCCACCGTGGCCTGGGTGACCCCCCAGCACCGGCCGGTGACGGCCACCAGCGCGGGCCGGGCGCGCGT
GCTCCCCGGGGGGACGCTGGAGATCCAGGACGCGCGGCCGCAGGACAGCGGCACCTACACGTGCGTGGCCAGCAACGCGG
GCGGCAACGACACCTACTTCGCCACGCTGACCGTGCGCCCCGAGCCGGCCGCCAACCGGACCCCGGGCGAGGCCCACAAC
GAGACGCTGGCGGCCCTGCGCGCGCCGCTCGACCTCACCACCATCCTGGTGTCCACCGCCATGGGCTGCATCACCTTCCT
GGGCGTGGTCCTCTTCTGCTTCGTGCTGCTGTTCGTGTGGAGCCGCGGCCGCGGGCAGCACAAAAACAACTTCTCGGTGG
AGTACTCCTTCCGCAAGGTGGATGGGCCGGCCGCCGCGGCGGGCCAGGGAGGCGCGCGCAAGTTCAACATGAAGATGATC
TGAGGGGTCCCCAGGGCGGA

The disclosed NOV3 nucleic acid sequence maps to chromosome 19 and has 917 of 1521 bases (60%) identical to an insulin-like growth factor binding mRNA from Papio (GENBANK-ID: S83462) (E=2.8e⁻⁴²).

A disclosed NOV3 protein (SEQ ID NO:6) encoded by SEQ ID NO:5 has 608 amino acid residues, and is presented using the one-letter code in Table 3B. Signal P, Psort and/or Hydropathy results predict that NOV3 contains a signal peptide, and is likely to be localized to the plasma membrane with a certainty of 0.4600. The most likely cleavage site for a NOV3 peptide is between amino acids 40 and 41, at: AGG-CP.

TABLE 3B
Encoded NOV3 protein sequence.

(SEQ ID NO:6)

MCAGGWWRGPRPTLRTMTCWLCVLSLPLLLLPAAPPPAGGCPARCECTVQTRAVACTRRRLTAVPDGIPAET
RLLELSRNRIRCLNPGDLAALPALEELDLSENAIAHVEPGAFANLPRLRVLRLRGNQLKLIPPGVFTRLDNL
TLLDLSENKLVILLDYTFQDLHSLRRLEVGDNDLVFVSRRAFAGLLALEELTLERCNLTALSGESLGHLRSL
GALRLRHLAIASLENQNFRRLPGLLHLEIDNWPLLEEVAAGSLRGLNLTSLSVTHTNITAVPAAALRHQAHL
TCLNLSHNPISTVPRGSFRDLVRLRELHLAGALLAVVEPQAFLGLRQIRLLNLSNNLLSTLEESTFHSVNTL
ETLRVDGNPLACDCRLLWIVQRRKTLNFDGRLPACATPAEVRGDALRNLPDSVLFEYFVCRKPKIRERRLQR
VTATAGEDVRFLCRAEGEPAPTVAWVTPQHRPVTATSAGRARVLPGGTLEIQDARPQDSGTYTCVASNAGGN
DTYFATLTVRPEPAANRTPGEAHNETLAALRAPLDLTTILVSTAMGCITFLGVVLFCFVLLFVWSRGRGQHK
NNFSVEYSFRKVDGPAAAAGQGGARKFNMKMI

The NOV3 amino acid sequence has 334 of 614 amino acid residues (54%) identical to, and 430 of 614 amino acid residues (70%) similar to, the Macaca fascicularis 614 amino acid residue hypothetical 69.2 kDa protein (ACC:BAB03557) (E=1.5e⁻¹⁶⁶). The global sequence homology is 62.396% amino acid homology and 54.576% amino acid identity.

NOV3 is expressed in at least the following tissues: Brain, anaplastic oligodendroglioma, and Colon. In addition, the NOV3 sequence is predicted to be expressed in the Liver because of the expression pattern of a closely related Papio insulin-like growth factor binding protein-3 complex acid-labile subunit homolog (GENBANK-ID: S83462).

NOV3 also has homology to the amino acid sequences shown in the BLASTP data listed in Table 3C.

TABLE 3C
BLAST results for NOV3

| Gene Index/ | | Length | Identity | Positives | |
|---|---|---|---|---|---|
| Identifier | Protein/Organism | (aa) | (%) | (%) | Expect |
| gi\|12309630\|emb\| | bA438B23.1 | 606 | 339/603 | 439/603 | 0.0 |
| CAC22713.1\| | (neuronal | | (56%) | (72%) | |
| (AL353746) | leucine-rich | | | | |
| | repeat protein) | | | | |
| | [Homo sapiens] | | | | |
| gi\|15301270\|ref\|XP_{—} | hypothetical | 614 | 333/621 | 427/621 | 1e−169 |
| 053144.1 | protein | | (53%) | (68%) | |
| | XP_053144 | | | | |
| | [Homo sapiens] | | | | |
| gi\|9651089\|dbj\| | hypothetical | 614 | 332/621 | 427/621 | 1e−168 |
| BAB03557.1 | protein | | (53%) | (68%) | |
| (AB046639) | [Macaca fascicularis] | | | | |
| gi\|12832048\|dbj\| | putative | 614 | 332/621 | 425/621 | 1e−168 |
| BAB32403.1\| | [Mus musculus] | | (53%) | (67%) | |
| (AK027262) | | | | | |
| gi\|14754729\|ref\|XP_{—} | hypothetical | 315 | 159/314 | 211/314 | 5e−75 |
| 047947.1 | protein | | (50%) | (66%) | |
| | FLJ14594 | | | | |
| | [Homo sapiens] | | | | |

The homology of these sequences is shown graphically in the ClustalW analysis shown in Table 3D.

[image]

[image]

[image]

Tables 3E-3G list the domain description from DOMAIN analysis results against NOV3. This indicates that the NOV3 sequence has properties similar to those of other proteins known to contain these domains.

TABLE 3E
Domain Analysis of NOV3

gnl Smart|smart00409, IG, Immunoglobulin (SEQ ID NO:81)
Length=86 residues, 97.7% aligned
Score=71.2 bits (173), Expect=2e-13

| | | | |
|---|---|---|---|
| NOV3 | 431 | GRVTATAGEDVRFLCRAEGEPAPTVAWVTPQHRPVTATSAGRARVLPG-GTLEIQDARPQ | 489 |
| | | **\|\|***\|\|*\|***\|*\|*\|*\|*\|\|\|*\|*****+*+**+*********\|**\|\|*\|*+**\|+ | |
| Smart00409 | 2 | PSVTVKEGESVTLSCEASGNPPPTVTWYKQGGKLLAESGRFSVSRSGGNSTLTISNVTPE | 61 |
| NOV3 | 490 | DSGTYTCVASNAGGNDTYFATLTV 513 | |
| | | \|\|\|\|\|\|\|*\|+\|+*\|+*+***\|\|\|\| | |
| Smart00409 | 62 | DSGTYTCAATNSSGSASSGTTLTV 85 | |

TABLE 3F
Domain Analysis of NOV3

gnl′Smart|smart00408, IGc2, Immunoglobulin C-2 Type (SEQ ID NO:82)
Length=63 residues, 96.8% aligned
Score=57.8 bits (138), Expect=2e-09

| | | | |
|---|---|---|---|
| NOV3 | 438 | GEDVRFLCRAEGEPAPTVAWTPQHRPVTATSAGRARVLPGGTLEIQDARPQDSGTYTCV | 497 |
| | | \|\|*\|***\|*\|*\|+\|*\|*+*\|+****************\|**\|*\|\|*\|++***+\|\|\|*\|\|\|\| | |
| Smart00408 | 3 | GESVTLTCPASGDPVPNITWLKDGKP----- LPESRVVASGSTLTIKNVSLEDSGLYTCV | 57 |
| NOV3 | 498 | ASNAGG 503 | |
| | | \|*\|+*\| | |
| Smart00408 | 58 | ARNSVG 63 | |

TABLE 3G
Domain Analysis of NOV3

gnl Pfam pfam0047, ig, Immunoglobulin domain. Members of the
immunoglobulin superfamily are found in hundreds of proteins of
different functions. Examples include antibodies, the giant muscle
kinase titin and receptor tyrosine kinases. Immunoglobulin-like
domains may be involved in protein-protein and protein-ligand
interactions. The Pfam alignments do not include the first and last
strand of the immunoglobulin-like domain. (SEQ ID NO:83)
Length=68 residues, 100.0% aligned
Score=43.5 bits (101), Expect=3e-05

| | | | |
|---|---|---|---|
| NOV3 | 438 | GEDVRFLCRAEG-EPAPTVAWVTPQHRPVTATSAGRARVLPGG-------TLEIQDARPQ | 489 |
| | | \|\|*\|***\|***\|**\|*\|\|\|*\|+***********+**+\|\|**\|\|*******+\|*\|****\|+ | |
| Pfam00047 | 1 | GESVTLTCSVSGYPPDPTVTWLRDGKEIELLGSSE-SRVSSGGRFSISSLSLTISSVTPE | 59 |
| NOV3 | 490 | DSGTYTCVA 498 | |
| | | \|\|\|\|\|\|\| | |
| pfam00047 | 60 | DSGTYTCVV 68 | |

Leucine rich-like proteins generally comprise leucine-rich repeats (LRRs), relatively short motifs (22-28 residues in length) found in a variety of cytoplasmic, membrane and extracellular proteins. Although theses proteins are associated with widely different functions, a common property involves protein-protein interaction. Although little is known about the 3-D structure of LRRs, it is believed that they can form amphipathic structures with hydrophilic surfaces capable of acting with membranes. In vitro studies of a synthetic LRR from Drosophila Toll protein have indicated that the peptides form gels by adopting beta-sheet structures that form extended filaments. These results are consistent with the idea that LRRs mediate protein-protein interactions and cellular adhesion. Other functions of LRR-containing proteins include, for example, binding to enzymes and vascular repair. The 3-D structure of ribonuclease inhibitor, a protein containing 15 LRRs, has been determined, revealing LRRs to be a new class of alpha/beta fold. LRRs form elongated non-globular structures and are often flanked by cysteine-rich domains.

Leucine-rich-like proteins have been shown to be involved in protein-protein interactions that result in protein complexes, receptor ligand binding or cell adhesion. Leucine rich-like proteins have been shown to be useful in potential therapeutic applications implicated in lymphatic diseases, skin and connective tissue diseases, diabetes and kidney diseases, cancers, tumors and brain disorders, disorders that can be addressed by controlling and directing cell migration, Alzheimer's disease, stroke, tuberous sclerosis, hypercalcemia, Parkinson's disease, Huntington's disease, cerebral palsy, epilepsy, Lesch-Nyhan syndrome, multiple sclerosis, ataxia telangiaectasia, leukodystrophies, behavioral disorders, addition, anxiety, pain, neuroprotection, inflammatory bowel disease, diverticular disease and Crohn's disease. These proteins and nucleic acids are further useful in the generation of antibodies for use in therapeutic or diagnostic methods.

The above defined information for NOV3 suggests that this leucine-rich protein may function as a member of a leucine-rich protein family. Therefore, the NOV3 nucleic acids and proteins of the invention are useful in potential therapeutic and diagnostic applications. For example, a cDNA encoding the NOV3 protein may be useful in gene therapy, and the NOV3 protein may be useful when administered to a subject in need thereof. By way of nonlimiting example, the compositions of the present invention will have efficacy for treatment of patients suffering from Lymphatic Diseases, Skin and Connective Tissue Diseases, Diabetes and Kidney Disease, Cancers, tumors, and Brain Disorders, disorders that can be addressed by controlling and directing cell migration, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, Inflammatory bowel disease, Diverticular disease, and Crohn's Disease. The NOV3 nucleic acid encoding leucine-rich protein, and the leucine-rich protein of the invention, or fragments thereof, may further be useful in diagnostic applications, wherein the presence or amount of the nucleic acid or the protein are to be assessed.

NOV4

A disclosed NOV4 nucleic acid of 1049 nucleotides (designated CuraGen Acc. No. GMba39917_A) encoding a novel cathepsin-L precursor-like protein is shown in Table 4A. An open reading frame was identified beginning with an ATG initiation codon at nucleotides 37-39 and ending with a TGA codon at nucleotides 1036-1038. Putative untranslated regions upstream from the initiation codon and downstream from the termination codon re underlined in Table 4A, and the start and stop codons are in bold letters.

TABLE 4A
NOV4 Nucleotide Sequence

(SEQ ID NO:7)

ATCCTCATTTCTTTTCCCTTCCTAGATTTTTGAAAATGGAATCCTTCACTCCTCCTGGCTGCCTTTTGCC
TGGGAATTGCCTCAGCTGCTCTAACACGTGACCACAGTTTAGACGCACAATGGACCAAGTGGAAGGCAAA
GCACAAGAGATTATATGGCATGAATGGAGAAGGATGGAGAAGGAGCTGTTGGGAGAAGGACGTGAAGATG
ATTGAGCAGCACAATCAGGAATACAGCCAAGGGAAACACAGCTTCACAATGGCCATGAACGCCTTTGGAG
ACATGGTAAGTGAAGAATTCAGGCAGGTGATGAATGGTTTTCAATACCAGAAGCACAGGAAGGGGAAACA
GTTCCAGGAACGCCTGCTTCCTGAGATCCCCACATCTGTGGACTGGAGAGAGAAAGGCTACATGACTCCT
GTGAAGGATCAGGGTCAGTGTGGCTCTTGTTGGGCTTTTAGTGCAACTGGTGCTCTGGAAGGGCAGATGT
TTTGGAAAACAGGCAAACTTATCTCACTGAATGAGCTCAATCTGGTAGACTGCTCTGGGCCTCAAGGCAA
TGAAGGCTGCAATGGTGGCTTGATGAACTATCATTTTGAATTTGTTCAGGACCACTCTGGGCAAGAAAGT
GAGACCTCATATCCTCTTGAAAGTAAGGTTAAAACCTGTAGGTACAATCCCAAGTATTCTGCTGCTAATG
ACACTGGTTTTGTGGACATCCCTTCACGGGAGAAGGACCTGGCGAAGGCAGTGGCAACTGTGGGGCCCAT
CTCTGTTGCTGTTGGTGCAAGCCATGTCTTCTTCCAGTTCTATAAAAAAGGTATTTATTTTGAGCCACGC
TGTGACCCTGAAGGCCTGGATCATGCTATGCTGGTGGTTGGCTACAGCTATGAAGGAGCAGACTCAGATA
ACAATAAATATTGGCTGGTGAAGAACAGCTGGGGTAAAAACTGGGGCATGGATGGCTACATAAAGATGGC
CAAGACCGGAGGAACAACTGTGGAATTGCCACAGCAGCCAGCTACCCCACTGTGTGAGCTGATGGATG

The nucleic acid sequence of NOV4, localized on chromosome 10, has 876 of 1022 bases (85%) identical to a Homo sapiens Cathepsin-L Precursor mRNA (GENBANK-ID: HSCATHL) (E=2.6e−¹⁶⁴).

A NOV4 polypeptide (SEQ ID NO:8) encoded by SEQ ID NO:7 is 333 amino acid residues and is presented using the one letter code in Table 4B. Signal P, Psort and/or Hydropathy results predict that NOV4 contains signal peptide and is likely to be localized at the plasma membrane with a certainty of 0.8200. The most likely cleavage site for a NOV4 peptide is between amino acids 17 and 18, at: ASA-AL.

TABLE 4B
NOV4 protein sequence

(SEQ ID NO:8)

MNPSLLLAAFCLGIASAALTRDHSLDAQWTKWKAKHKRLYGMNGEGWRRSCWEKDVKMIEQHNQEYS
QGKHSFTMAMNAFGDMVSEEFRQVMNGFQYQKHRKGKQFQERLLPEIPTSVDWREKGYMTPVKDQGQ
CGSCWAFSATGALEGQMFWKTGKLISLNELNLVDCSGPQGNEGCNGGLMNYHFEFVQDHSGQESETS
YPLESKVKTCRYNPKYSAANDTGFVDIPSREKDLAKAVATVGPISVAVGASHVFFQFYKKGIYFEPR
CDPEGLDHAMLVVGYSYEGADSDNNKYWLVKNSWGKNWGMDGYIKMAKDRRNNCGIATAASYPTV

The NOV4 amino acid sequence has 256 of 33 amino acid residues (76%) identical to, and 288 of 333 residues (86%) positive with, the Homo sapiens 333 amino acid residue Cathepsin-L Precursor protein (P07711) (E=2.1e−144). The global sequence homology is 80.781% amino acid homology and 76.877% amino acid identity.

NOV4 is expressed in at least the following tissues: Musculoskeletal System, Bone, Female Reproductive System, Placenta, Endocrine System, Adrenal Gland/Suprarenal gland, Respiratory System, Lung, Hematopoietic and Lymphatic System, Hematopoietic Tissues, Lymphoid tissue, Spleen, Gastro-intestinal/Digestive System, Liver, Whole Organism, Cardiovascular System, Adipose, Nervous System, Brain, Male Reproductive System, Testis. In addition, NOV4 is predicted to be expressed in the following tissues because of the expression pattern of a closely related Sus scrofa cathepsin L precursor homolog (GENBANK-ID: PIGPCL): Musculoskeletal System, Bone, Female Reproductive System, Placenta, Endocrine System, Adrenal Gland/Suprarenal gland, Respiratory System, Lung, Hematopoietic and Lymphatic System, Hematopoietic Tissues, Lymphoid tissue, Spleen, Gastro-intestinal/Digestive System, Liver, Whole Organism, Cardiovascular System, Adipose, Nervous System, Brain, Male Reproductive System and Testis.

NOV4 also has homology to the amino acid sequences shown in the BLASTP data listed in Table 4C.

TABLE 4C
BLAST results for NOV4

| Gene Index/ | | Length | Identity | Positives | |
|---|---|---|---|---|---|
| Identifier | Protein/Organism | (aa) | (%) | (%) | Expect |
| gi\|15214962\|gb\| | Similar to | 333 | 257/333 | 288/333 | 1e−153 |
| AAH12612.1\| | cathepsin L | | (77%) | (86%) | |
| AAH12612 | [Homo sapiens] | | | | |
| (BC012612) | | | | | |
| gi\|4503155\|ref\|NP_{—} | cathepsin L | 333 | 256/333 | 288/333 | 1e−152 |
| 001903.1 | [Homo sapiens] | | (76%) | (85%) | |
| gi\|11493685\|gb\| | cysteine protease | 333 | 252/333 | 285/333 | 1e−150 |
| AAG35605.1\| | [Cercopithecus aethiops] | | (75%) | (84%) | |
| AF201700_1 | | | | | |
| (AF201700) | | | | | |
| gi\|5822035\|pdb\| | Chain A, Crystal | 316 | 239/316 | 270/316 | 1e−140 |
| 1CS8A | Structure Of | | (75%) | (84%) | |
| | Procathepsin L | | | | |
| gi\|10185020\|emb\| | cathepsin L | 333 | 243/334 | 276/334 | 1e−140 |
| CAC08809.1\| | [Canis familiaris] | | (72%) | (81%) | |
| (AJ279008) | | | | | |

The homology of these sequences is shown graphically in the ClustalW analysis shown in Table 4D.

[image]

[image]

Tables 4E and 4F list the domain description from DOMAIN analysis results against NOV4. This indicates that the NOV4 sequence has properties similar to those of other proteins known to contain these domains.

TABLE 4E
Domain Analysis of NOV4

gnl|pfam,pfam00112, Peptidase_C1, Papain family cysteine protease
(SEQ ID NO:89)
Length = 220 residues, 100.0% aligned
Score = 266 bits (680), Expect = 1e=72

| | | |
|---|---|---|
| NOV4 | 114 | IPTSVDWREKG-YMTRVKDQGQCGSCWAESATGALEGQMFWKTG-KLISLNELNLVDCSG 171 |
| | | +\| \| \|\|\|+\|\| +\|\|\|\|\|\|\|\|\|\|\|\|\|\|\|\|\| \|\|\|\|\|+ \|\|\| \|\|+\|\|+\| \|\|\|\|\|\| |
| pFAM00112 | 1 | LPESFDWRDKGGAVTPVKDQGQCGSCWAFSAVGALEGRYCIKTGGKLVSLSEQQLVDCSG 60 |
| NOV4 | 172 | PQGNEGCNGGLMNYHFEFVQDHSGQESETSYPLESK-VKTCRYNPKYS--AANDTGFVDI 228 |
| | | \| \|\|\|\|\|\| + \|\|++ \| +\|+ \|\| \| \|\|+ \| \| \| \| \|+ \|+ |
| pfam00112 | 61 | D--NNGCNGGLPDNAFEYIIKG-GLPTESDYPYTGKDGGTCKKNCKNSKNYAKIKGYGDV 117 |
| NOV4 | 229 | PSR-EKDLAKAVATVGPISVAVGASHVFFQFYKKGIYFEPRCDPEGLDHAMLVVGYSYEG 287 |
| | | \| \|+ \| \|+\|\| \|\|+\|\|\|+ \| \|\| \|\| \|\|\| \| \| \|\|\|\|+\|+\|\|\| + |
| Pfam00112 | 118 | PYNDEEALQAALATNGPVSVAIDAYEDDFQLYKSGIYKHTECGGENLDHAVLIVGYGTD- 176 |
| NOV4 | 288 | ADSDNNKYWLVKNSWGKNWGMDGYIKMAKDRRNNCGIATAASYPT 332 |
| | | \|\|+\|\|\|\|\|\| +\|\| +\|\| ++\|+ \| \|\|\|\|+ \|\|\|\| |
| pfam00112 | 177 | -GDGGKPYWIVKNSWGTDWGENGYFRIARGGNNECGIASEASYPI 220 |

TABLE 4F
Domain Analysis of NOV4

gnl Smart|smart00645, Pept_C1, Papain family cysteine protease
(SEQ ID NO:90)
Length = 218 residues, 100.0% aligned
Score = 251 bits (640), Expect 6e−68

| | | |
|---|---|---|
| NOV4 | 114 | IPTSVDWREKGYMTPVKDQGQCGSCWAFSATGALEGQMFWKTG-KLISLNELNLVDCSGP 172 |
| | | +\| \| \|\|\|+\|\| +\|\|\|\|\|\|\|\|\|\|\|\|\|\|\|\|\|\|\|\|\|\|\|+ \|\|\| \|\|+\|\|+\| \|\|\|\|\|\| |
| Smart0645 | 1 | LPESFDWRKKGAVTPVKDQGQCGSCWAFSATGALEGRYCIKTGGKLVSLSEQQLVDCSGG 60 |
| NOV4 | 172 | QGNEGCNGGLMNYHFEFVQDHSGQESETSYPLESK-VKTCRYNPKYSAA---NDTGFVDI 228 |
| | | \|\| \|\|\|\|\|\| + \|\|+++ + \| +\|+ \|\| \| \| \| \|\| \| \|+ |
| Smart0645 | 61 | -GNNGCNGGLPDNAFEYIKKNGGLGTESCYPYTGKDGGPCPYTPKCSKKCVSGIKGYDVP 119 |
| NOV4 | 229 | PSREKDLAKAVATVGPISVAVGASHVFFQFYKKGIYFEPRCDPEGLDHAMLVVGYSYEGA 288 |
| | | + \|+ \| +\|\|\| \|\|+\|\|\|+ \|\| \|\|\|\|\| \|\|\| \| \| \|+\|\|+\|+\|\|\| \| |
| Smart0645 | 120 | YNDEEILKEAVANGGPVSVAIDASD--FQFYKSGIYDHPGCGSGLLNHAVLIVGY---GT 174 |
| NOV4 | 289 | DSDNNKYWLVKNSWGKNWGMDGYIKMAKDRRNNCGI-ATAASYP 331 |
| | | + \|\|+\|\|\|\|\|\| +\|\| +\|\| ++\|+ \| \|\|\| \|+ \|\|\|\| |
| Smart0645 | 175 | SENGKDYWIVKNSWGTDWGENGYFRIARGVNNECGIEASVASYP 218 |

Cathepsins are lysosomal proteases that are distributed in many normal tissues and are primarily responsible for intracellular catabolism and turnover. Studies suggest that cathepsin-L may have some roles in terminal differentiation (PMID: 10699763, UI 20164186). Cathepsin-L, a lysosomal cysteine proteinase belongs to the papain family. This proteinase is different from other members of the mammalian papain family cysteine proteinase in the following ways: (i) the cathepsin-L gene is activated by a variety of growth factors and activated oncogenes, (ii) procathepsin-L, a precursor form of cathepsin L is secreted from various cells, (iii) the mRNA level of cathepsin-L is related to the in vivo metastatic potential of the transformed cells. Thus, the regulation of the cathepsin-L gene and the extracellular functions of secreted procathepsin-L are tightly coupled. (PMID: 9524064, UI:98182239).

Studies also suggest that cathepsin-L may have some roles in the terminal differentiation (PMID: 10699763, UI: 20164186). The increased level of cathepsins in tumors together with their ability to degrade extracellular matrix protein has led to the hypothesis that they are involved in the process of invasion and metastasis. In 8 cases of dermatofibrosarcoma protuberans (DFS), five cases of atypical fibroxanthoma (AFX) and twenty cases of dermatofibroma (DF). Expression of cathepsins B and pro-D could be detected in 5 of the 8 cases (62.5%) of DFS, whereas cathepsin pro-L was found in 4 (50%) cases. All AFX expressed cathepsin pro-L, whereas cathepsins B and pro-D were observed in 4 out of 5 cases. None of the malignant tumors showed a recurrence or metastasis after a period of four years. No expression of cathepsins in DF was found. In the epidermis and appendages, an expression of cathepsins pro-D, pro-L and B was seen. Cathepsins may be markers of increased metabolism rather than specific markers of malignancy (PMID: 9649659, UI: 99075963).

The above defined information for NOV4 suggests that this NOV4 protein may function as a member of a cathepsin-L precursor-like protein family. Therefore, the NOV4 nucleic acids and proteins of the invention are useful in potential therapeutic applications implicated in various diseases and disorders described below and/or other pathologies. For example, the NOV4 compositions of the present invention will have efficacy for treatment of patients suffering from growth of soft tissue sarcomas; cathepsin L is induced in tumors by malignant transformation, growth factors, and tumor promoters suggesting they play an important role in tumor invasion and metastasis. Additionally, cathepsin L may be involved in bone resorption implicating possible roles in bone diseases such as osteoporosis, or bone cancers. Additional disorders include Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Transplantation, Adrenoleukodystrophy, Congenital Adrenal Hyperplasia, Diabetes, Von Hippel-Lindau (VHL) syndrome, Pancreatitis, Endometriosis, Fertility, Inflammatory bowel disease, Diverticular disease, Hirschsprung's disease, Crohn's Disease, Hemophilia, hypercoagulation, Idiopathic thrombocytopenic purpura, immunodeficiencies, Osteoporosis, Hypercalceimia, Arthritis, Ankylosing spondylitis, Scoliosis, Endocrine dysfunctions, Diabetes, Growth and reproductive disorders, Psoriasis, Actinic keratosis, Acne, Hair growth, allopecia, pigmentation disorders, endocrine disorders. The NOV4 nucleic acid encoding cathepsin-L precursor-like protein, and the cathepsin-L precursor-like protein of the invention, or fragments thereof, may further be useful in diagnostic applications, wherein the presence or amount of the nucleic acid or the protein are to be assessed.

NOV5

A disclosed NOV5 nucleic acid of 491 nucleotides (also referred to as GMba38118_A) encoding a novel fatty acid-binding protein-like protein is shown in Table 5A. An open reading frame was identified beginning with an ATG initiation codon at nucleotides 10-12 and ending with a TAA codon at nucleotides 462-464. Putative untranslated regions upstream from the initiation codon and downstream from the termination codon are underlined in Table 5A, and the start and stop codons are in bold letters.

TABLE 5A
NOV5 Nucleotide Sequence

(SEQ ID NO:9)

CATGCTGCCATGCCGACGTAGACCCTGCTCTGCACGCCAGCCCGCCCGCA
CCCACCATGGCCACAGTTCAGCAGCTGGAAGGAAGATGGCGCCTGCTGGA
CAGCAAAGGCTTTGATGAATACATGAAGGAGCTAGGAGTGGGAATAGCTT
TGCAAAAAATGGGCGCAATGGCCAAGCCAGATTGTATCACTTGTACCCTG
GGAGATGAGTTTGAAGAAACCACAGCTGATGGCAGAAAAACACAGACTGT
CTGCAACTTTACAGATGGTGCATTGGTTCAGCATCAGGAGTGGGATGGGA
AGGAAAGCACAATAACAAGAAAATTGAAAGATGGGAAATTAGTGGTGGAG
TGTGTCATGAACAATGTCACCTGTACTCGGATCTATGAAAAAGTAGAATA
AAAATTCCATCATCACTTTGGACAGGAG

The NOV5 nucleic acid was identified on chromosome 13 and has 458 of 480 bases (97%) identical to a Homo sapiens Fatty Acid-Binding Protein mRNA (GENBANK-ID: HUMFABPHA) (E=1.9e⁹⁷)

A disclosed NOV5 polypeptide (SEQ ID NO:10) encoded by SEQ ID NO:9 is 135 amino acid residues and is presented using the one-letter code in Table 5B. Signal P, Psort and/or Hydropathy results predict that NOV5 does not have a signal peptide and is likely to be localized in the cytoplasm with a certainty of 0.6500.

TABLE 5B
Encoded NOV5 protein sequence

(SEQ ID NO:10)

MATVQQLEGRWRLLDSKGFDEYMKELGVGIALQKMGAMAKPDCIITCDGR
NLTTKTESTLKTTQFSCTLGDEFEETTADGRKTQTVCNFTDGALVQHQEW
DGKESTITRKLKDGKLVVECVMNNVTCTRIYEKVE

The NOV5 amino acid sequence has 129 of 135 amino acid residues (95%) identical to, and 134 of 135 residues (99%) similar to, the Homo sapiens 135 amino acid residue Fatty Acid-Binding protein Q01469 (E=6.1e⁻⁶⁷). The global sequence homology is 97.037% amino acid similarity and 95.556% amino acid identity.

NOV5 is expressed in at least the following tissues: Sensory System, Skin, Nervous System, Brain, Male Reproductive System, Testis, Respiratory System, Lung, Larynx, Female Reproductive System, Placenta, Whole Organism, Cardiovascular System, Heart, Endocrine System, Parathyroid Gland, Hematopoietic and Lymphatic System, Hematopoietic Tissues, Liver, Tonsils, Gastro-intestinal/Digestive System, Large Intestine, Colon, Stomach, Oesophagus, Urinary System, Kidney. In addition, the NOV5 is predicted to be expressed in the following tissues because of the expression pattern of a closely related Mus musculus Fatty Acid-Binding Protein homolog (GENBANK-ID: ACC:Q05816): Sensory System, Skin, Nervous System, Brain, Male Reproductive System, Testis, Respiratory System, Lung, Larynx, Female Reproductive System, Placenta, Whole Organism, Cardiovascular System, Heart, Endocrine System, Parathyroid Gland, Hematopoietic and Lymphatic System, Hematopoietic Tissues, Liver, Tonsils, Gastro-intestinal/Digestive System, Large Intestine, Colon, Stomach, Oesophagus, Urinary System and Kidney.

NOV5 also has homology to the amino acid sequences shown in the BLASTP data listed in Table 5C.

TABLE 5C
BLAST results for NOV5

| Gene Index/ | | Length | Identity | Positives | |
|---|---|---|---|---|---|
| Identifier | Protein/Organism | (aa) | (%) | (%) | Expect |
| gi\|13651563\|ref\|XP_{—} | similar to | 135 | 135/135 | 135/135 | 2e−65 |
| 015760.1\| | GASTRIN/CHOLECYST | | (100%) | (100%) | |
| | OKININ TYPE B | | | | |
| | RECEPTOR (CCK-B | | | | |
| | RECEPTOR) | | | | |
| | [Homo sapiens] | | | | |
| gi\|4557581\|ref\|NP_{—} | fatty acid | 135 | 129/135 | 134/135 | 3e−63 |
| 001435.1\| | binding protein 5 | | (95%) | (98%) | |
| | (psoriasis- | | | | |
| | associated) | | | | |
| | [Homo sapiens] | | | | |
| gi\|13651468\|ref\|XP_{—} | similar to | 135 | 125/135 | 132/135 | 6e−63 |
| 016351.1\| | GASTRIN/ | | (92%) | (97%) | |
| | CHOLECYST | | | | |
| | OKININ TYPE B | | | | |
| | RECEPTOR (CCK-B | | | | |
| | RECEPTOR) | | | | |
| | [Homo sapiens] | | | | |
| gi\|13651882\|ref\|XP_{—} | fatty acid | 135 | 120/135 | 130/135 | 1e−59 |
| 011655.5\| fatty | binding protein 5 | | (88%) | (95%) | |
| acid binding | (psoriasis- | | | | |
| protein 5 | associated) | | | | |
| (psoriasis- | [Homo sapiens] | | | | |
| associated) | | | | | |
| [Homo sapiens] | | | | | |
| gi\|14746180\|ref\|XP_{—} | similar to | 135 | 119/135 | 128/135 | 5e−59 |
| 018419.2\| | GASTRIN/CHOLECYST | | (88%) | (94%) | |
| | OKININ TYPE B | | | | |
| | RECEPTOR (CCK-B | | | | |
| | RECEPTOR) | | | | |
| | [Homo sapiens] | | | | |

The homology of these sequences is shown graphically in the ClustalW analysis shown in Table 5D.

[image]

[image]

Table 5E list the domain description from DOMAIN analysis results against NOV5. This indicates that the NOV5 sequence has properties similar to those of other proteins known to contain this domain.

TABLE 5E
Domain Analysis of NOV5

gnl.Pfam.pfam00061, lipocalin, Lipocalin/cytosolic fatty-acid
binding protein family. Lipocalins are transporters for small
hydrophobic molecules, such as lipids, steroid hormones, bilins, and
retinoids. Alignment subsumes both the lipocalin and fatty acid
binding protein signatures from PROSITE. This is supported on
structural and functional grounds. Structure is an eight-stranded beta
barrel. (SEQ ID NO:96)
Length = 145 residues, 100.0% aligned
Score = 47.8 bits (112), Expect = 4e−07

| | | |
|---|---|---|
| NOV5 | 6 | QLEGRWRLLDSKGFDEYMK-ELGVGIALQKMGAMAK-PDCIITCDGRNLTTKTESTLKTT 63 |
| | | + \|+\| \|+ \| \|\| +\| \|\|\|\| \| +\| \| + \| \|\| \|+ \| |
| Pfam00061 | 1 | KFAGKWYLVASANFDPELKEELGVLEATRKEITPLKEGNLEIVFDGDKNGICEETFGKLE 60 |
| NOV5 | 64 | QFSCTLGDEFEETTADGRKTQTVCNFTDGALVQHQEWDGKESTITRKLKDG--------- 114 |
| | | + \|\| \|\|+ \| \| \| ++ + \|\| \|+ \|\| \|++ \| +\| |
| Pfam00061 | 61 | KTK-KLGVEFDYYTGDNRFVVLDTDYDNYLLVCVQKGDGNETSRTAELYGRTPELSPEAL 119 |
| N0V5 | 115 | KILVVECV------NNVTCTRIYEKV 134 |
| | | +\| +\|\| \|\|\| \|+ |
| Pfam00061 | 120 | ELFETATKELGIPEDNVVCTRQTERC 145 |

Fatty acid metabolism in mammalian cells depends on a flux of fatty acids, between the plasma membrane and mitochondria or peroxisomes for beta-oxidation, and between other cellular organelles for lipid synthesis. The fatty acid-binding protein (FABP) family consists of small, cytosolic proteins believed to be involved in the uptake, transport, and solubilization of their hydrophobic ligands. Members of this family have highly conserved sequences and tertiary structures. Fatty acid-binding proteins were first isolated in the intestine (FABP2; OMIM-134640) and later found in liver (FABP1; OMIM-134650), striated muscle (FABP3; OMIM-134651), adipocytes (FABP4; OMIM-600434) and epidermal tissues (E-FABP; GDB ID:136450).

Epidermal fatty acid binding protein (E-FABP) was cloned by as a novel keratinocyte protein by Madsen et al (1992, PMID: 1512466) from skin of psoriasis patients. Later using quantitative Western blot analysis, Kingma et al. (1998, PMID: 9521644) have shown that in addition to the skin, bovine E-FABP is expressed in retina, testis, and lens. Since E-FABP was originally identified from the skin of psoriasis patients, it is also known as psoriasis-associated fatty acid-binding protein (PA-FABP). PA-FABP is a cytoplasmic protein, and is expressed in keratinocytes. It is highly up-regulated in psoriatic skin. It shares similarity to other members of the fatty acid-binding proteins and belongs to the fabp/p2/crbp/crabp family of transporter. PA-FABP is believed to have a high specificity for fatty acids, with highest affinity for c18 chain length. Decreasing the chain length or introducing double bonds reduces the affinity. PA-FABP may be involved in keratinocyte differentiation.

Immunohistochemical localization of the expression of E-FABP in psoriasis, basal and squamous cell carcinomas has been carried out in order to obtain indirect information, at the cellular level, on the transport of the fatty acids. (Masouye et al, 1996, PMID: 8726632). E-FABP was localized in the upper stratum spinosum and stratum granulosum in normal and non-lesional psoriatic skin. In contrast, lesional psoriatic epidermis strongly expressed E-FABP in all suprabasal layers, like nonkeratinized oral mucosa. The basal layer did not express E-FABP reactivity in any of these samples. Accordingly, basal cell carcinomas were E-FABP negative whereas only well-differentiated cells of squamous cell carcinomas expressed E-FABP. This suggests that E-FABP expression is related to the commitment of kerafinocyte differentiation and that the putative role of E-FABP should not be restricted to the formation of the skin lipid barrier. Since the pattern of E-FABP expression mimics cellular FA transport, our results suggest that lesional psoriatic skin and oral mucosa have a higher metabolism/transport for FAs than normal and non-lesional psoriatic epidermis.

The above defined information for NOV5 suggests that this NOV5 protein may function as a member of a fatty acid-binding protein family. Therefore, the NOV5 nucleic acids and proteins of the invention are useful in potential therapeutic applications implicated in various diseases and disorders described below and/or other pathologies. For example, the NOV5 compositions of the present invention will have efficacy for treatment of patients suffering from psoriasis, basal and squamous cell carcinomas, obesity, diabetes, and/or other pathologies and disorders involving fatty acid transport of skin, oral mucosa as well as other organs, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Transplantation, Adrenoleukodystrophy, Congenital Adrenal Hyperplasia, Diabetes, Von Hippel-Lindau (VHL) syndrome, Pancreatitis, Endometriosis, Fertility, Inflammatory bowel disease, Diverticular disease, Hirschsprung's disease, Crohn's Disease, Hemophilia, hypercoagulation, Idiopathic thrombocytopenic purpura, immunodeficiencies, Osteoporosis, Hypercalceimia, Arthritis, Ankylosing spondylitis, Scoliosis, Endocrine dysfunctions, Diabetes, Growth and reproductive disorders, Psoriasis, Actinic keratosis, Acne, Hair growth, allopecia, pigmentation disorders and endocrine disorders. The NOV5 nucleic acid encoding fatty acid-binding protein, and the fatty acid-binding protein of the invention, or fragments thereof, may further be useful in diagnostic applications, wherein the presence or amount of the nucleic acid or the protein are to be assessed.

NOV6

NOV6 includes nine novel neurolysin precursor-like proteins disclosed below. The disclosed proteins have been named NOV6a, NOV6b, NOV6c, NOV6d, NOV6e, NOV6f, NOV6g, NOV6h and NOV6i.

NOV6a

A disclosed NOV6a nucleic acid of 2170 nucleotides (also referred to as SC1 33790496_A) encoding a novel neurolysin precursor-like protein is shown in Table 6A. An open reading frame was identified beginning with an ATG initiation codon at nucleotides 16-18 and ending with a TGA codon at nucleotides 2128-2130. Putative untranslated regions upstream from the initiation codon and downstream from the termination codon are underlined in Table 6A, and the start and stop codons are in bold letters.

TABLE 6A
NOV6a Nucleotide Sequence

(SEQ ID NO:11)

CCTCTCAGCGCTCCCATGATCGCCCGGTGCCTTTTGGCTGTGCGAAGCCTCCGCAGGGTTGGTGGTTCCA
GGATTTTACTCAGAATGACGTTAGGAAGAGAAGTGATGTCTCCTCTTCAGGCAATGTCTTCCTATACTGT
GGCTGGCAGAAATGTTTTAAGATGGGATCTTTCACCAGAGCAAATTAAAACAAGAACTGAGGAGCTCATT
GTGCAGACCAAACAGGTGTACGATGCTGTTGGAATGCTCGGTATTGAGGAAGTAACTTACGAGAACTGTC
TGCAGGCACTGGCAGATGTAGAAGTAAAGTATATAGTGGAAAGGACCATGCTAGACTTTCCCCAGCATGT
ATCCTCTGACAAAGAAGTACGAGCAGCAAGTACAGAAGCAGACAAAAGACTTTCTCGTTTTGATATTGAG
ATGAGCATGAGAGGAGATATATTTGAGAGAATTGTTCATTTACAGGAAACCTGTGATCTGGGGAAGATAA
AACCTGAGGCCAGACGATACTTGGAAAAGTCAATTAAAATGGGGAAAAGAAATGGGCTCCATCTTCCTGA
ACAAGTACAGAATGAAATCAAATCAATGAAGAAAAGAATGAGTGAGCTATGTATTGATTTTAACAAAAAC
CTCAATGAGGATGATACCTTCCTTGTATTTTCCAAGGCTGAACTTGGTGCTCTTCCTGATGATTTCATTG
ACAGTTTAGAAAAGACAGATGATGACAAGTATAAAATTACCTTAAAATATCCACACTATTTCCCTGTCAT
GAAGAAATGTTGTATCCCTGAAACCAGAAGAAGGATGGAAATGGCTTTTAATACAAGGTGCAAAGAGGAA
AACACCATAATTTTGCAGCAGCTACTCCCACTGCGAACCAAGGTGGCCAAACTACTCGGTTATAGCACAC
ATGCTGACTTCGTCCTTGAAATGAACACTGCAAAGAGCACAAGCCGCGTAACAGCCTTTCTAGATGATTT
AAGCCAGAAGTTAAAACCCTTGGGTGAAGCAGAACGAGAGTTTATTTTGAATTTGAAGAAAAAGGAATGC
AAAGACAGGGGTTTTGAATATGATGGGAAAATCAATGCCTGGGATCTATATTACTACATGACTCAGACAG
AGGAACTCAAGTATTCCATAGACCAAGAGTTCCTCAAGGAATACTTCCCAATTGAGGTGGTCACTGAAGG
CTTGCTGAACACCTACCAGGAGTTGTTGGGACTTTCATTTGAACAAATGACAGATGCTCATGTTTGGAAC
AAGAGTGTTACACTTTATACTGTGAAGGATAAAGCTACAGGAGAAGTATTGGGACAGTTCTATTTGGACC
TCTATCCAAGGGAAGGAAAATACAATCATGCGGCCTGCTTCGGTCTCCAGCCTGGCTGCCTTCTGCCTGA
TGGAAGCCGGATGATGGCAGTGGCTGCCCTCGTGGTGAACTTCTCACAGCCAGTGGCAGGTCGTCCCTCT
CTCCTGAGACACGACGAGGTGAGGACTTACTTTCATGAGTTTGGTCACGTGATGCATCAGATTTGTGCAC
AGACTGATTTTGCACGATTTAGCGGAACAAATGTGGAAACTGACTTTGTAGAGGTGCCATCGCAAATGCT
TGAAAATTGGGTGTGGGACGTCGATTCCCTCCGAAGATTGTCAAAACATTATAAAGATGGAAGCCCTATT
GCAGACGATCTGCTTGAAAAACTTGTTGCTTCGCTTATGTTATTAGGTCTTCTGACCCTGCGCCAGATTG
TTTTGAGCAAAGTTGATCAGTCTCTTCATACCAACACATCGCTGGATGCTGCAAGTGAATATGCCAAATA
CTGCTCAGAAATATTAGGAGTTGCAGCTACTCCAGGTACAAATATGCCAGCTACCTTTGGACATTTGGCA
GGGGGATACGATGGCCAATATTATGGATATCTTTGGAGTGAAGTATTTTCCATGGATATGTTTTACAGCT
GTTTTAAAAAAGAAGGGATAATGAATCCAGAGGTAGTTGGAATGAAATACAGAAACCTAATCCTGAAACC
TGGGGGATCTCTGGACGGCATGGACATGCTCCACAATTTCTTGAAACGTGAGCCAAACCAAAAAGCGTTC
CTAATGAGTAGAGGCCTGCATGCTCCGTGAACTGGGGATCTTTGGTAGCCGTCCATGTCTGGAGGACAAG

The disclosed NOV6a nucleic acid sequence was identified on chromosome 5 and has 1994 of 2170 (91%) identical to a Sus scrofa Neurolysin Precursor mRNA (GENBANK-ID: PIGSABP) (E=0.0).

A disclosed NOV6a polypeptide (SEQ ID NO:12) encoded by SEQ ID NO:11 is 704 amino acid residues and is presented using the one-letter amino acid code in Table 6B. Signal P, Psort and/or Hydropathy results predict that NOV6a contains a signal peptide and is likely to be localized at the plasma membrane with a certainty of 0.7000. The most likely cleavage site for a NOV6a peptide is between amino acids 17 and 18, at: VGG-SR.

TABLE 6B
Encoded NOV6a protein sequence.

(SEQ ID NO:12)

MIARCLLAVRSLRRVGGSRILLRMTLGREVMSPLQAMSSYTVAGRNVLRWDLSPEQIKTRTEELIVQTKQVYDAVG
MLGIEEVTYENCLQALADVEVKYIVERTMLDFPQHVSSDKEVRAASTEADKRLSRFDIEMSMRGDIFERIVHLQET
CDLGKIKPEARRYLEKSIKMGKRNGLHLPEQVQNEIKSMKKRMSELCIDFNKNLNEDDTFLVFSKAELGALPDDFI
DSLEKTDDDKYKITLKYPHYFPVMKKCCIPETRRRMEMAFNTRCKEENTIILQQLLPLRTKVAKLLGYSTHADFVL
KEYFPIEVVTEGLLNTYQELLGLSFEQMTDAHVWNKSVTLYTVKDKATGEVLGQFYLDLYPREGKYNHAACFGLQP
GCLLPDGSRMMAVAALVVNFSQPVAGRPSLLRHDEVRTYFHEFGHVMHQICAQTDFARFSGTNVETDFVEVPSQML
ENWVWDVDSLRRLSKHYKDGSPIADDLLEKLVASLMLLGLLTLRQIVLSKVDQSLHTNTSLDAASEYAKYCSELLG
VAATPGTNMPATFGHLAGGYDGQYYGYLWSEVFSMDMFYSCFKKEGIMNPEVVGMKYRNLILKPGGSLDGMDMLHN
FLKREPNQKAFLMSRGLHAHP

The NOV6a amino acid sequence has 661 of 704 amino acid residues (93%) identical to, and 667 of 704 amino acid residues (96%) similar to, the Sus scrofa 704 amino acid residue Neurolysin Precursor protein (Q02038) (E=0.0). The global sequence homology is 95.164% amino acid homology and 94.026% amino acid identity.

NOV6a is expressed in at least the following tissues: Whole Organism, Sensory System, Skin, Foreskin, Gastro-intestinal/Digestive System, Large Intestine, Colon, Salivary Glands, Cardiovascular System, Vein, Umbilical Vein, Female Reproductive System, Uterus, Nervous System, Brain, Prosencephalon/Forebrain, Diencephalon, Thalamus, Cardiovascular System, Artery, Coronary Artery, Heart, Male Reproductive System and Prostate. In addition, NOV6a is predicted to be expressed in the following tissues because of the expression pattern of a closely related Sus scrofa Neurolysin Precursor homolog (GENBANK-ID: PIGSABP): Whole Organism, Sensory System, Skin, Foreskin, Gastro-intestinal/Digestive System, Large Intestine, Colon, Salivary Glands, Cardiovascular System, Vein, Umbilical Vein, Female Reproductive System, Uterus, Nervous System, Brain, Prosencephalon/Forebrain, Diencephalon, Thalamus, Cardiovascular System, Artery, Coronary Artery, Heart, Male Reproductive System and Prostate.

NOV6a also has homology to the amino acid sequences shown in the BLASTP data listed in Table 6C.

TABLE 6C
BLAST results for NOV6a

| Gene Index/ | | Length | Identity | Positives | |
|---|---|---|---|---|---|
| Identifier | Protein/Organism | (aa) | (%) | (%) | Expect |
| gi\|417743\|sp\| | NEUROLYSIN | 704 | 661/705 | 677/705 | 0.0 |
| Q02038\| | PRECURSOR | | (93%) | (95%) | |
| NEUL_PIG | (NEUROTENSIN | | | | |
| | ENDOPEPTIDASE) | | | | |
| | (MITOCHONDRIAL | | | | |
| | OLIGOPEPTIDASE M | | | | |
| | [Sus scrofa] | | | | |
| gi\|14149738\|ref\|NP_{—} | neurolysin; | 704 | 700/705 | 701/705 | 0.0 |
| 065777.1\| | KIAA1226 protein; | | (99%) | (99%) | |
| | neurotensin | | | | |
| | endopeptidase | | | | |
| | [Homo sapiens] | | | | |
| gi\|1171691\|sp\| | NEUROLYSIN | 704 | 626/703 | 667/703 | 0.0 |
| P42676\| | PRECURSOR | | (89%) | (94%) | |
| NEUL_RAT | (NEUROTENSIN | | | | |
| | ENDOPEPTIDASE) | | | | |
| | (MITOCHONDRIAL | | | | |
| | OLIGOPEPTIDASE M) | | | | |
| | [Rattus | | | | |
| | norvegicus] | | | | |
| gi\|1783127\|dbj\| | endopeptidase | 745 | 652/691 | 668/691 | 0.0 |
| BAA19063.1\| | 24.16 type M2 | | (94%) | (96%) | |
| (AB000172) | [Sus scrofa] | | | | |
| gi\|1783123\|dbj\| | endopeptidase | 681 | 644/682 | 660/682 | 0.0 |
| BAA19061.1\| | 24.16 type M3 | | (94%) | (96%) | |
| (AB000170) | [Sus scrofa] | | | | |

The homology of these sequences is shown graphically in the ClustalW analysis shown in Table 6D.

[image]

[image]

[image]

Table 6E lists the domain description from DOMAIN analysis results against NOV6a. This indicates that the NOV6a sequence has properties similar to those of other proteins known to contain this domain.

TABLE 6E
Domain Analysis of NOV6a

gnl|Pfam|pfam01432, Peptidase_M3, Peptidase family M3. This is the
Thimet oligopeptidase family, large family of mammalian and bacterial
oligopeptidases that cleave medium sized peptides. The group also
contains mitochondrial intermediate peptidase which is encoded by
nuclear DNA but functions within the mitochondria to remove the leader
sequence. (SEQ ID NO:102)
Length = 603 residues, 100.0% aligned
Score = 617 bits (1592), Expect = 5e−178

| | | |
|---|---|---|
| NOV6a | 88 | CLQALADVEVKYIVERTMLDFPQHVSSDKEVRAASTEADKRLSRFDIEMSMRGDIFERIV 147 |
| | | \|+\|\| ++\| + +\| \| \|\|\|+ \|+ \|\| ++\|\| +\|+\| \|++ \|+ |
| Pfam01432 | 1 | TLKALDELEDTLCRVYDLGEFLQSAHPDKELLEAAEEASEKLSELMNYLSLREDLYTRLK 60 |
| NOV6a | 148 | HLQ-ETCDLGKIKPEARRYLEKSIKMGKRNGLHLPEQVQNEIKSMKKRMSELCIDFNKNL 206 |
| | | + + + \|\|\|\|\| +\|\| \| +++\|+ \|\|\|+ + + \| +\|\| + \|\| \| \| \|\|\| |
| Pfam01432 | 61 | AVLDDKSKSESLDPEARRVVEKFEKDFEKSGIGLPEEKREKFKLIKKELKELGIAFEKNL 120 |
| NOV6a | 207 | NEDDTFLVFSKAELGALPDDFIDSLEKTDDDKYKITLKYPHYFPVMKKCCIPETRRRMEM 266 |
| | | \| \| \|++ +\| \|\|+ + \| \|\|\| ++ \|\| \|\| \|+\|\| \| \|\|\| ++ |
| Pfam01432 | 121 | REKKHLLSFTEEKLAGLPEPVLASAEKTFEELGN-TLAYPT-LPLMKYCENNETREKLYS 178 |
| NOV6a | 267 | AFNTRCKEENTIILQQLLPLRTKVAKLLGYSTHADFVLEMNTAKSTSRVTAFLDDLSQKL 326 |
| | | \|++ \| + \|\| \| ++ \| \|\| ++\| \|\|\| +\|+\|+ +\|\| \|\|+ \| \|\|\| \| \| |
| pfam01432 | 179 | AYHNRLESENRAIRKEALKLRAELAYLLGRNTYANLLLEDKMAKNPEAVLRFLDSLRSKA 238 |
| NOV6a | 327 | KPLGEAEREFILNLKKKECKDRGFEYDGKINAWDLYYYMTQTEELKYSIDQEFLKEYFPI 386 |
| | | \| \| \| \| \|\|\|\| ++ \|\| \|\| + \| \|\|\|+\| \| \|\| \|\|\|+ |
| pfam01432 | 239 | LPNNEIELAVIDELKKKEL------GVNELLPWDHRYYSLRYREEKYSLDPELLKPYFPL 292 |
| NOV6a | 387 | EVVTEGLLNTYQELLGLSFEQMTDAHVWNKSVTLYTVKDKATGEVLGQFYLDLYPREGKY 446 |
| | | + \|\|\| ++\|\| \|\|+\|\|+ \| \|\|+ \| \| \| \|+ \|\|+\|\|\|\|\|\| \| \| |
| Pfam01432 | 293 | TPLIEGLFRLFKELYGLTFEEAADGEVWHPDVRLGEVYDEILKGALGEFYLDLYARRGGK 352 |
| NOV6a | 447 | NHAACFGLQPGCLLPDGSRMMAVAALVVNFSQPVAGRPSLLRHDEVRTYFHEFGHVMHQI 506 |
| | | \|\| \| \| \| + \|\| \|+ \|\|++\| \|\|+\|\|\|\| \|\|+\| \| \|\|\|\|\|\| \|\| + |
| Pfam01432 | 353 | RTGACSGG--GSLDG----QLPVAYLLCNFTKPSAGKPSLLTHDDVFTLFHEFGHSMHSM 406 |
| NOV6a | 507 | CAQTDFARFSGTNVETDFVEVPSQMLENWVWDVDSLRRLSKHYKDGSPIADDLLEKLVAS 566 |
| | | ++\| ++ \|\|\| \| \|\|\|\|+\|\| + \|\|\|+\|+ \| \|\|\|\|\|\| \| \|\| \|+\|\|\| \|+ |
| Pfam01432 | 407 | LSRTHYSYVSGTYVPIDFVEIPSILNENWLWEPLLLNLLSKHYKTGEPIPDELLEKFFAT 466 |
| NOV6a | 567 | LM-LLGLLTLRQIVLSKVDQSLHTNTSLDAASEYAKYCSEILGVAAT--PGTNMPATFGH 623 |
| | | \| \| \|\|+ + +\|\| \|\| \| \| \|\|+ + \|++\| \|\|\| \| \| \| |
| Pfam01432 | 467 | KFRQTGFATFEQIIHALLDQGLHHLTEEDLTEIYAELNEKYFGLSAVDKPGTLWWARFPH 526 |
| NOV6a | 624 | LAGGYDGQYYGYLWSEVFSMDMFYSCFKKEGIMNPEVVGMKYRNLILKPGGSLDGMDMLH 683 |
| | | \|\|\| \|\| \|\|++ + \|+\| + \| \|+\| +\| \| \|++\|\| \| \|\|\| \| ++\|\| |
| Pfam01432 | 527 | FYGGYAANYYVYLYATGLAADLFLAKFIKDGDLNRE-NGVRYRKEFLSSGGSKDPLEMLK 585 |
| NOV6a | 684 | NFLKREPNQKAFLMSRGL 701 |
| | | \|\| \|\|++ \|\| + \|\| |
| Pfam01432 | 586 | KFLGDEPSKDPFLEANGL 603 |

Novel variants for the NOV6a nucleic acid and Neurolysin Precursor-like protein sequences are also disclosed herein as variants of NOV6a. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, however, in the case that a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern for example, alteration in temporal expression, physiological response regulation, cell type expression regulation, intensity of expression, stability of transcribed message. Variants are reported individually, but any combination of all or a subset are also included.

A disclosed NOV6b nucleic acid (also referred to as 13375342) is a variant of NOV6a, encodes a novel neurolysin precursor-like protein, and is shown in Table 6F. NOV6b nucleotide changes are underlined in Table 6F.

TABLE 6F
NOV6b Nucleotide Sequence

(SEQ ID NO:13)

CCTCTCAGCGCTCCCATGATCGCCCGGTGCCTTTTGGCTGTGCGAAGCCTCCGCAGGGTTGGTGGTTCCAGGATTTTAC
TCAGAATGACGTTAGGAAGAGAAGTGATGTCTCCTCTTCAGGCAATGTCTTCCTATACTGTGGCTGGCAGAAATGTTTT
AAGATGGGATCTTTCACCAGAGCAAATTAAAACAAGAACTGAGGAGCTCATTGTGCAGACCAAACAGGTGTACGATGCT
GTTGGAATGCTCGGTATTGAGGAAGTAACTTACGAGAACTGTCTGCAGGCACTGGCAGATGTAGAAGTAAAGTATATAG
TGGAAAGGACCATGCTAGACTTTCCCCAGCATGTATCCTCTGACAAAGAAGTACGAGCAGCAAGTACAGAAGCAGACAA
AAGACTTTCTCGTTTTGATATTGAGATGAGCATGAGAGGAGATATATTTGAGAGAATTGTTCATTTACAGGAAACCTGT
GATCTGGGGAAGATAAAACCTGAGGCCAGACGATACTTGGAAAAGTCAATTAAAATGGGGAAAAGAAATGGGCTCCATC
TTCCTGAACAAGTACAGAATGAAATCAAATCAATGAAGAAAAGAATGAGTGAGCTATGTATTGATTTTAACAAAAACCT
CAATGAGGATGATACCTTCCTTGTATTTTCCAAGGCTGAACTTGGTGCTCTTCCTGATGATTTCATTGACAGTTTAGAA
AAGACAGATGATGACAAGTATAAAATTACCTTAAAATATCCACACTATTTCCCTGTCATGAAGAAATGTTGTATCCCTG
AAACCAGAAGAAGGATGGAAATGGCTTTTAATACAAGGTGCAAAGAGGAAAACACCATAATTTTGCAGCAGCTACTCCC
ACTGCGAACCAAGGTGGCCAAACTACTCGGTTATAGCACACATGCTGACTTCGTCCTTGAAATGAACACTGCAAAGAGC
ACAAGCCGCGTAACGGCCTTTCTAGATGATTTAAGCCAGAAGTTAAAACCCTTGGGTGAAGCAGAACGAGAGTTTATTT
TGAATTTGAAGAAAAAGGAATGCAAAGACAGGGGTTTTGAATATGATGGGAAAATCAATGCCTGGGATCTATATTACTA
CATGACTCAGACAGAGGAACTCAAGTATTCCATAGACCAAGAGTTCCTCAAGGAATACTTCCCAATTGAGGTGGTCACT
GAAGGCTTGCTGAACACCTACCAGGAGTTGTTGGGACTTTCATTTGAACAAATGACAGATGCTCATGTTTGGAACAAGA
GTGTTACACTTTATACTGTGAAGGATAAAGCTACAGGAGAAGTATTGGGACAGTTCTATTTGGACCTCTATCCAAGGGA
AGGAAAATACAATCATGCGGCCTGCTTCGGTCTCCAGCCTGGCTGCCTTCTGCCTGATGGAAGCCGGATGATGGCAGTG
GCTGCCCTCGTGGTGAACTTCTCACAGCCAGTGGCAGGTCGTCCCTCTCTCCTGAGACACGACGAGGTGAGGACTTACT
TTCATGAGTTTGGTCACGTGATGCATCAGATTTGTGCACAGACTGATTTTGCACGATTTAGCGGAACAAATGTGGAAAC
TGACTTTGTAGAGGTGCCATCGCAAATGCTTGAAAATTGGGTGTGGGACGTCGATTCCCTCCGAAGATTGTCAAAACAT
TATAAAGATGGAAGCCCTATTGCAGACGATCTGCTTGAAAAACTTGTTGCTTCGCTTATGTTATTAGGTCTTCTGACCC
TGCGCCAGATTGTTTTGAGCAAAGTTGATCAGTCTCTTCATACCAACACATCGCTGGATGCTGCAAGTGAATATGCCAA
ATACTGCTCAGAAATATTAGGAGTTGCAGCTACTCCAGGTACAAATATGCCAGCTACCTTTGGACATTTGGCAGGGGGA
TACGATGGCCAATATTATGGATATCTTTGGAGTGAAGTATTTTCCATGGATATGTTTTACAGCTGTTTTAAAAAAGAAG
GGATAATGAATCCAGAGGTAGTTGGAATGAAATACAGAAACCTAATCCTGAAACCTGGGGGATCTCTGGACGGCATGGA
CATGCTCCACAATTTCTTGAAACGTGAGCCAAACCAAAAAGCGTTCCTAATGAGTAGAGGCCTGCATGCTCCGTGAACT
GGGGATCTTTGCTAGCCGTCCATGTCTGGAGGACAAG

A disclosed NOVb polypeptide (SEQ ID NO:14) encoded by SEQ ID NO:13 is presented using the one-letter amino acid code in Table 6G. NOV6b amino acid changes, if any, are underlined in Table 6G.

TABLE 6G
Encoded NOV6b protein sequence.

(SEQ ID NO:14)

MIARCLLAVRSLRRVGGSRILLRMTLGREVMSPLQAMSSYTVAGRNVLRWDLSPEQIKTRTEELIVQTKQVYDAVGMLGIEEVTY
ENCLQALADVEVKYIVERTMLDFPQHVSSDKEVRAASTEADKRLSRFDIEMSMRGDIFERIVHLQETCDLGKIKPEARRYLEKSI
KMGKRNGLHLPEQVQNEIKSMKKRMSELCIDFNKNLNEDDTFLVFSKAELGALPDDFIDSLEKTDDDKYKITLKYPHYFPVMKKC
CIPETRRRMEMAFNTRCKEENTIILQQLLPLRTKVAKLLGYSTHADFVLEMNTAKSTSRVTAFLDDLSQKLKPLGEAEREFILNL
KKKECKDRGFEYDGKINAWDLYYYMTQTEELKYSIDQEFLKEYFPIEVVTEGLLNTYQELLGLSFEQMTDAHVWNKSVTLYTVKD
KATGEVLGQFYLDLYPREGKYNHAACFGLQPGCLLPDGSRMMAVAALVVNFSQPVAGRPSLLRHDEVRTYFHEFGHVMHQICAQT
DFARFSGTNVETDFVEVPSQMLENWVWDVDSLRRLSKHYKDGSPIADDLLEKLVASLMLLGLLTLRQIVLSKVDQSLHTNTSLDA
ASEYAKYCSEILGVAATPGTNMPATFGHLAGGYDGQYYGYLWSEVFSMDMFYSCFKKEGIMNPEVVGMKYRNLILKPGGSLDGMD
MLHNFLKREPNQKAFLMSRGLHAP

A disclosed NOV6c nucleic acid (also referred to as c99.456) is a variant of NOV6a, encodes a novel neurolysin precursor-like protein, and is shown in Table 6H. NOV6c nucleotide changes are underlined in Table 6H.

TABLE 6H
NOV6c Nucleotide Sequence

(SEQ ID NO:15)

CCTCTCAGCGCTCCCATGATCGCCCGGTGCCTTTTGGCTGTGCGAAGCCTCCGCAGGGTTGGTGGTTCCAGGATTTTAC
TCAGAATGACGTTAGGAAGAGAAGTGATGTCTCCTCTTCAGGCAATGTCTTCCTATACTGTGGCTGGCACAAATGTTTT
AAGATGGGATCTTTCACCAGAGCAAATTAAAACAAGAACTGAGGAGCTCATTGTGCAGACCAAACAGGTGTACGATGCT
GTTGGAATGCTCGGTATTGAGGAAGTAACTTACGAGAACTGTCTGCAGGCACTGGCAGATGTAGAAGTAAAGTATATAG
TGGAAAGGACCATGCTAGACTTTCCCCAGCATGTATCCTCTGACAAAGAAGTACGAGCAGCAAGTACAGAAGCAGACAA
AAGACTTTCTCGTTTTGATATTGAGATGAGCATGAGAGGAGATATATTTGAGAGAATTGTTCATTTACAGGAAACCTGT
GATCTGGGGAAGATAAAACCTGAGGCCAGACGATACTTGGAAAAGTCAATTAAAATGGGGAAAAGAAATGGGCTCCATC
TTCCTGAACAAGTACAGAATGAAATCAAATCAATGAAGAAAAGAATGAGTGAGCTATGTATTGATTTTAACAAAAACCT
CAATGAGGATGATACCTTCCTTGTATTTTCCAAGGCTGAACTTGGTGCTCTTCCTGATGATTTCATTGACAGTTTAGAA
AAGACAGATGATGACAAGTATAAAATTACCTTAAAATATCCACACTATTTCCCTGTCATGAAGAAATGTTGTATCCCTG
AAACCAGAAGAAGGATGGAAATGGCTTTTAATACAAGGTGCAAAGAGGAAAACACCATAATTTTGCAGCAGCTACTCCC
ACTGCGAACCAAGGTGGCCAAACTACTCGGTTATAGCACACATGCTGACTTCGTCCTTGAAATGAACACTGCAAAGAGC
ACAAGCCGCGTAACAGCCTTTCTAGATGATTTAAGCCAGAAGTTAAAACCCTTGGGTGAAGCAGAACGAGAGTTTATTT
TGAATTTGAAGAAAAAGGAATGCAAAGACAGGGGTTTTGAATATGATGGGAAAATCAATGCCTGGGATCTATATTACTA
CATGACTCAGACAGAGGAACTCAAGTATTCCATAGACCAAGAGTTCCTCAAGGAATACTTCCCAATTGAGGTGGTCACT
GAAGGCTTGCTGAACACCTACCAGGAGTTGTTGGGACTTTCATTTGAACAAATGACAGATGCTCATGTTTGGAACAAGA
GTGTTACACTTTATACTGTGAAGGATAAAGCTACAGGAGAAGTATTGGGACAGTTCTATTTGGACCTCTATCCAAGGGA
AGGAAAATACAATCATGCGGCCTGCTTCGGTCTCCAGCCTGGCTGCCTTCTGCCTGATGGAAGCCGGATGATGGCAGTG
GCTGCCCTCGTGGTGAACTTCTCACAGCCAGTGGCAGGTCGTCCCTCTCTCCTGAGACACGACGAGGTGAGGACTTACT
TTCATGAGTTTGGTCACGTGATGCATCAGATTTGTGCACAGACTGATTTTGCACGATTTAGCGGAACAAATGTGGAAAC
TGACTTTGTAGAGGTGCCATCGCAAATGCTTGAAAATTGGGTGTGGGACGTCGATTCCCTCCGAAGATTGTCAAAACAT
TATAAAGATGGAAGCCCTATTGCAGACGATCTGCTTGAAAAACTTGTTGCTTCGCTTATGTTATTAGGTCTTCTGACCC
TGCGCCAAATTGTTTTGAGCAAAGTTGATCAGTCTCTTCATACCAACACATCGCTGGATGCTGCAAGTGAATATGCCAA
ATACTGCTCAGAAATATTAGGAGTTGCAGCTACTCCAGGTACAAATATGCCAGCTACCTTTGGACATTTGGCAGGGGGA
TACGATGGCCAATATTATGGATATCTTTGGAGTGAAGTATTTTCCATGGATATGTTTTACAGCTGTTTTAAAAAAGAAG
GGATAATGAATCCAGAGGTAGTTGGAATGAAATACAGAAACCTAATCCTGAAACCTGGGGGATCTCTGGACGGCATGGA
CATGCTCCACAATTTCTTGAAACGTGAGCCAAACCAAAAAGCGTTCCTAATGAGTAGAGGCCTGCATGCTCCGTGAACT
GGGGATCTTTGGTAGCCGTCCATGTCTGGAGGACAAG

A disclosed NOV6c polypeptide (SEQ ID NO:16) encoded by SEQ ID NO:15 is presented using the one-letter amino acid code in Table 61. NOV6c amino acid changes, if any, are underlined in Table 6I.

TABLE 6I
Encoded NOV 6c protein sequence.

(SEQ ID NO:16)

MIARCLLAVRSLRRVGGSRILLRMTLGREVMSPLQAMSSYTVAGRNVLRWDLSPEQIKTRTEELIVQTKQVYDAVGMLGIEEVTY
ENCLQALADVEVKYIVERTMLDFPQHVSSDKEVRAASTEADKRLSRFDIEMSMRGDIFERIVHLQETCDLGKIKPEARRYLEKSI
KMGKRNGLHLPEQVQNEIKSMKKRMSELCIDFNKNLNEDDTFLVFSKAELGALPDDFIDSLEKTDDDKYKITLKYPHYFPVMKKC
CIPETRRRMEMAFNTRCKEENTIILQQLLPLRTKVAKLLGYSTHADFVLEMNTAKSTSRVTAFLDDLSQKLKPLGEAEREFILNL
KKKECKDRGFEYDGKINAWDLYYYMTQTEELKYSIDQEFLKEYFPIEVVTEGLLNTYQELLGLSFEQMTDAHVWNKSVTLYTVKD
KATGEVLGQFYLDLYPREGKYNHAACFGLQPGCLLPDGSRMMAVAALVVNFSQPVAGRPSLLRHDEVRTYFHEFGHVMHQICAQT
DFARFSGTNVETDFVEVPSQMLENWVWDVDSLRRLSKHYKDGSPIADDLLEKLVASLMLLGLLTLRQIVLSKVDQSLHTNTSLDA
ASEYAKYCSEILGVAATPGTNMPATFGHLAGGYDGQYYGYLWSEVFSMDMFYSCFKKEGIMNPEVVGMKYRNLILKPGGSLDGMD
MLHNFLKREPNQKAFLMSRGLHAP

A disclosed NOV6d nucleic acid (also referred to as c99.457) is a variant of NOV6a, encodes a novel neurolysin precursor-like protein, and is shown in Table 6J. NOV6d nucleotide changes are underlined in Table 6J.

TABLE 6J
NOV6d Nucleotide Sequence

(SEQ ID NO:17)

CCTCTCAGCGCTCCCATGATCGCCCGGTGCCTTTTGGCTGTGCGAAGCCTCCGCAGGGTTGGTGGTTCCAGGATTTTAC
TCAGAATGACGTTAGGAAGAGAAGTGATGTCTCCTCTTCAGGCAATGTCTTCCTATACTGTGGCTGGCAGAAATGTTTT
AAGATGGGATCTTTCACCAGAGCAAATTAAAACAAGAACTGAGGAGCTCATTGTGCAGACCAAACAGGTGTACGATGCT
GTTGGAATGCTCGGTATTGAGGAAGTAACTTACGAGAACTGTCTGCAGGCACTGGCAGATGTAGAAGTAAAGTATATAG
TGGAAAGGACCATGCTAGACTTTCCCCAGCATGTATCCTCTGACAAAGAAGTACGAGCAGCAAGTACAGAAGCAGACAA
AAGACTTTCTCGTTTTGATATTGAGATGAGCATGAGAGGAGATATATTTGAGAGAATTGTTCATTTACAGGAAACCTGT
GATCTGGGGAAGATAAAACCTGAGGCCAGACGATACTTGGAAAAGTCAATTAAAATGGGGAAAAGAAATGGGCTCCATC
TTCCTGAACAAGTACAGAATGAAATCAAATCAATGAAGAAAAGAATGAGTGAGCTATGTATTGATTTTAACAAAAACCT
CAATGAGGATGATACCTTCCTTGTATTTTCCAAGGCTGAACTTGGTGCTCTTCCTGATGATTTCATTGACAGTTTAGAA
AAGACAGATGATGACAAGTATAAAATTACCTTAAAATATCCACACTATTTCCCTGTCATGAAGAAATGTTGTATCCCTG
AAACCAGAAGAAGGATGGAAATGGCTTTTAATACAAGGTGCAAAGAGGAAAACACCATAATTTTGCAGCAGCTACTCCC
ACTGCGAACCAAGGTGGCCAAACTACTCGGTTATAGCACACATGCTGACTTCGTCCTTGAAATGAACACTGCAAAGAGC
ACAAGCCCGCGTAACAGCCTTTCTAGATGATTTAAGCCAGAAGTTAAAACCCTTGGGTGAAGCAGAACGAGAGTTATTT
TGAATTTGAAGAAAAAGGAATGCAAAGACAGGGGTTTTGAATATGATGGGAAAATCAATGCCTGGGATCTATATTACTA
CATGACTCAGACAGAGGAACTCAAGTATTCCATAGACCAAGAGTTCCTCAAGGAATACTTCCCAATTGAGGTGGTCACT
GAAGGCTTGCTGAACACCTACCAGGAGTTGTTGGGACTTTCATTTGAACAAATGACAGATGCTCATGTTTGGAACAAGA
GTGTTACACTTTATACTGTGAAGGATAAAGCTACAGGAGAAGTATTGGGACAGTTCTATTTGGACCTCTATCCAAGGGA
AGGAAAATACAATCATGCGGCCTGCTTCGGTCTCCAGCCTGGCTGCCTTCTGCCTGATGGAAGCCGGATGATGGCAGTG
GCTGCCCTCGTGGTGAACTTCTCACAGCCAGTGGCAGGTCGTCCCTCTCTCCTGAGACACGACGAGGTGAGGACTTACT
TTCATGAGTTTGGTCACGTGATGCATCAGATTTGTGCACAGACTGATTTTGCACGATTTAGCGGAACAAATGTGGAAAC
TGACTTTGTAGAGGTGCCATCGCAAATGCTTGAAAATTGGGTGTGGGACGTCGATTCCCTCCGAAGATTGTCAAAACAT
TATAAAGATGGAAGCCCTATTGCAGACGATCTGCTTGAAAAACTTGTTGCTTCGCTTATGTTATTAGGTCTTCTGACCC
TGCGCCAGATTGTTTTGAGCAAAGTTGACCAGTCTCTTCATACCAACACATCGCTGGATGCTGCAAGTGAATATGCCAA
ATACTGCTCAGAAATATTAGGAGTTGCAGCTACTCCAGGTACAAATATGCCAGCTACCTTTGGACATTTGGCAGGGGGA
TACGATGGCCAATATTATGGATATCTTTGGAGTGAAGTATTTTCCATGGATATGTTTTACAGCTGTTTTAAAAAAGAAG
GGATAATGAATCCAGAGGTAGTTGGAATGAAATACAGAAACCTAATCCTGAAACCTGGGGGATCTCTGGACGGCATGGA
CATGCTCCACAATTTCTTGAAACGTGAGCCAAACCAAAAAGCGTTCCTAATGAGTAGAGGCCTGCATGCTCCGTGAACT
GGGGATCTTTGGTAGCCGTCCATGTCTGGAGGACAAG

A disclosed NOV6d polypeptide (SEQ ID NO:18) encoded by SEQ ID NO:17 is presented using the one-letter amino acid code in Table 6K. NOV6d amino acid changes, if any, are underlined in Table 6K.

TABLE 6K
Encoded NOV6d protein sequence.

(SEQ ID NO:18)

MIARCLLAVRSLRRVGGSRILLRMTLGREVMSPLQAMSSYTVAGRNVLRWDLSPEQIKTRTEELIVQTKQVYDAVGMLGIEEVTY
ENCLQALADVEVKYIVERTMLDFPQHVSSDKEVRAASTEADKRLSRFDIEMSMRGDIFERIVHLQETCDLGKIKPEARRYLEKSI
KMGKRNGLHLPEQVQNEIKSMKKRMSELCIDFNKNLNEDDTFLVFSKAELGALPDDFIDSLEKTDDDKYKITLKYPHYFPVMKKC
CIPETRRRMEMAFNTRCKEENTIILQQLLPLRTKVAKLLGYSTHADFVLEMNTAKSTSRVTAFLDDLSQKLKPLGEAEREFILNL
KKKECKDREGFEYDGKINAWDLYYMTQTEELKYSIDQEFLKEYFPIEVVTEGLLNTYQELLGLSFEQMTDAHVWNKSVTLYTVKD
KATGEVLGQFYLDLYPREGKYNHAACFGLQPGCLLPDGSRMMAVAALVVNFSQPVAGRPSLLRHDEVRTYFHEFGHVMHQICAQT
DFARFSGTNVETDFVEVPSQMLENWVWDVDSLRRLSKHYKDGSPIADDLLEKLVASLMLLGLLTLRQIVLSKVDQSLHTNTSLDA
ASEYAKYCSEILGVAATPGTNMPATFGHLAGGYDGQYYGYLWSEVFSMDMFYSCFKKEGIMNPEVVGMKYRNLILKPGGSLDGMD
MLHNFLKREPNQKAFLMSRGLHAP

A disclosed NOV6e nucleic acid (also referred to as c99.458) is a variant of NOV6a, encodes a novel neurolysin precursor-like protein, and is shown in Table 6L. NOV6e nucleotide changes are underlined in Table 6L.

TABLE 6L
NOV6e Nucleotide Sequence

(SEQ ID NO:19)

CCTCTCAGCGCTCCCATGATCGCCCGGTGCCTTTTGGCTGTGCGAAGCCTCCGCAGGGTTGGTGGTTCCAGGATTTTAC
TCAGAATGACGTTAGGAAGAGAAGTGATGTCTCCTCTTCAGGCAATGTCTTCCTATACTGTGGCTGGCAGAAATGTTTT
AAGATGGGATCTTTCACCAGAGCAAATTAAAACAAGAACTGAGGAGCTCATTGTGCAGACCAAACAGGTGTACGATGCT
GTTGGAATGCTCGGTATTGAGGAAGTAACTTACGAGAACTGTCTGCAGGCACTGGCAGATGTAGAAGTAAAGTATATAG
TGGAAAGGACCATGCTAGACTTTCCCCAGCATGTATCCTCTGACAAAGAAGTACGAGCAGCAAGTACAGAAGCAGACAA
AAGACTTTCTCGTTTTGATATTGAGATGAGCATGAGAGGAGATATATTTGAGAGAATTGTTCATTTACAGGAAACCTGT
GATCTGGGGAAGATAAAACCTGAGGCCAGACGATACTTGGAAAAGTCAATTAAAATGGGGAAAAGAAATGGGCTCCATC
TTCCTGAACAAGTACAGAATGAAATCAAATCAATGAAGAAAAGAATGAGTGAGCTATGTATTGATTTTAACAAAAACCT
CAATGAGGATGATACCTTCCTTGTATTTTCCAAGGCTGAACTTGGTGCTCTTCCTGATGATTTCATTGACAGTTTAGAA
AAGACAGATGATGACAAGTATAAAATTACCTTAAAATATCCACACTATTTCCCTGTCATGAAGAAATGTTGTATCCCTG
AAACCAGAAGAAGGATGGAAATGGCTTTTAATACAAGGTGCAAAGAGGAAAACACCATAATTTTGCAGCAGCTACTACC
ACTGCGAACCAAGGTGGCCAAACTACTCGGTTATAGCACACATGCTGACTTCGTCCTTGAAATGAACACTGCAAAGAGC
ACAAGCCGCGTAACAGCCTTTCTAGATGATTTAAGCCAGAAGTTAAAACCCTTGGGTGAAGCAGAACGAGAGTTTATTT
TGAATTTGAAGAAAAAGGAATGCAAAGACAGGGGTTTTGAATATGATGGGAAAATCAATGCCTGGGATCTATATTACTA
CATGACTCAGACAGAGGAACTCAAGTATTCCATAGACCAAGAGTTCCTCAAGGAATACTTCCCAATTGAGGTGGTCACT
GAAGGCTTGCTGAACACCTACCAGGAGTTGTTGGGACTTTCATTTGAACAAATGACAGATGCTCATGTTTGGAACAAGA
GTGTTACACTTTATACTGTGAAGGATAAAGCTACAGGAGAAGTATTGGGACAGTTCTATTTGGACCTCTATCCAAGGGA
AGGAAAATACAATCATGCGGCCTGCTTCGGTCTCCAGCCTGGCTGCCTTCTGCCTGATGGAAGCCGGATGATGGCAGTG
GCTGCCCTCGTGGTGAACTTCTCACAGCCAGTGGCAGGTCGTCCCTCTCTCCTGAGACACGACGAGGTGAGGACTTACT
TTCATGAGTTTGGTCACGTGATGCATCAGATTTGTGCACAGACTGATTTTGCACGATTTAGCGGAACAAATGTGGAAAC
TGACTTTGTAGAGGTGCCATCGCAAATGCTTGAAAATTGGGTGTGGGACGTCGATTCCCTCCGAAGATTGTCAAAACAT
TATAAAGATGGAAGCCCTATTGCAGACGATCTGCTTGAAAAACTTGTTGCTTCGCTTATGTTATTAGGTCTTCTGACCC
TGCGCCAGATTGTTTTGAGCAAAGTTGATCAGTCTCTCCATACCAACACATCGCTGGATGCTGCAAGTGAATATGCCAA
ATACTGCTCAGAAATATTAGGAGTTGCAGCTACTCCAGGTACAAATATGCCAGCTACCTTTGGACATTTGGCAGGGGGA
TACGATGGCCAATATTATGGATATCTTTGGAGTGAAGTATTTTCCATGGATATGTTTTACAGCTGTTTTAAAAAAGAAG
GGATAATCAATCCAGAGGTAGTTGGAATGAAATACAGAAACCTAATCCTGAAACCTGGGGGATCTCTGGACGGCATGGA
CATGCTCCACAATTTCTTGAAACGTGAGCCAAACCAAAAAGCGTTCCTAATGAGTAGAGGCCTGCATGCTCCGTGAACT
GGGGATCTTTGGTAGCCGTCCATGTCTGGAGGACAAG

A disclosed NOV6e polypeptide (SEQ ID NO:20) encoded by SEQ ID NO:19 is presented using the one-letter amino acid code in Table 6M. NOV6e amino acid changes, if any, are underlined in Table 6M.

TABLE 6M
Encoded NOV6e protein sequence.

(SEQ ID NO:20)

MIARCLLAVRSLRRVGGSRILLRMTLGREVMSPLQAMSSYTVAGRNVLRWDLSPEQIKTRTEELIVQTKQVYDAVGMLGIEEVTY
ENCLQALADVEVKYIVERTMLDFPQHVSSDKEVRAASTEADKRLSRFDIEMSMRGDIFERIVHLQETCDLGKIKPEARRYLEKSI
KMGKRNGLHLPEQVQNEIKSMKKRMSELCIDFNKNLNEDDTFLVFSKAELGALPDDFIDSLEKTDDDKYKITLKYPHYFPVMKKC
CIPETRRRMEMAFNTRCKEENTIILQQLLPLRTKVAKLLGYSTHADFVLEMNTAKSTSRVTAFLDDLSQKLKPLGEAEREFILNL
KKKECKDRGFEYDGKINAWDLYYYMTQTEELKYSIDQEFLKEYFPIEVVTEGLLNTYQELLGLSFEQMTDAHVWNKSVTLYTVKD
KATGEVLGQFYLDLYPREGKYNHAACFGLQPGCLLPDGSRMMAVAALVVNFSQPVAGRPSLLRHDEVRTYFHEFGHVMHQICAQT
DFARFSGTNVETDFVEVPSQMLENWVWDVDSLRRLSKHYKDGSPIADDLLEKLVASLMLLGLLTLRQIVLSKVDQSLHTNTSLDA
ASEYAKYCSEILGVAATPGTNMPATFGHLAGGYDGQYYGYLWSEVFSMDMFYSCFKKEGIMNPEVVGMKYRNLILKPGGSLDGMD
MLHNFLKREPNQKAFLMSRGLHAP

A disclosed NOV6f nucleic acid (also referred to as 13375341) is a variant of NOV6a, encodes a novel neurolysin precursor-like protein, and is shown in Table 6N. NOV6f nucleotide changes are underlined in Table 6N.

TABLE 6N
NOV6f Nucleotide Sequence

(SEQ ID NO:21)

CCTCTCAGCGCTCCCATGATCGCCCGGTGCCTTTTGGCTGTGCGAAGCCTCCGCAGGGTTGGTGGTTCCAGGATTTTAC
TCAGAATGACGTTAGGAAGAGAAGTGATGTCTCCTCTTCAGGCAATGTCTTCCTATACTGTGGCTGGCAGAAATGTTTT
AAGATGGGATCTTTCACCAGAGCAAATTAAAACAAGAACTGAGGAGCTCATTGTGCAGACCAAACAGGTGTACGATGCT
GTTGGAATGCTCGGTATTGAGGAAGTAACTTACGAGAACTGTCTGCAGGCACTGGCAGATGTAGAAGTAAAGTATATAG
TGGAAAGGACCATGCTAGACTTTCCCCAGCATGTATCCTCTGACAAAGAAGTACGAGCAGCAAGTACAGAAGCAGACAA
AAGACTTTCTCGTTTTGATATTGAGATGAGCATGAGAGGAGATATATTTGAGAGAATTGTTCATTTACAGGAAACCTGT
GATCTGGGGAAGATAAAACCTGAGGCCAGACGATACTTGGAAAAGTCAATTAAAATGGGGAAAAGAAATGGGCTCCATC
TTCCTGAACAAGTACAGAATGAAATCAAATCAATGAAGAAAAGAATGAGTGAGCTATGTATTGATTTTAACAAAAACCT
CAATGAGGATGATACCTTCCTTGTATTTTCCAAGGCTGAACTTGGTGCTCTTCCTGATGATTTCATTGACAGTTTAGAA
AAGACAGATGATGACAAGTATAAAATTACCTTAAAATATCCACACTATTTCCCTGTCATGAAGAAATGTTGTATCCCTG
AAACCAGAAGAAGGATGGAAATGGCTTTTAATACAAGGTGCAAAGAGGAAAACACCATAATTTTGCAGCAGCTACTCCC
ACTGCGAACCAAGGTGGCCAAACTACTCGGTTATAGCACACATGCTGACTTCGTCCTTGAAATGAACACTGCAAAGAGC
ACAAGCCGCGTAACAGCCTTTCTAGATGATTTAAGCCAGAAGTTAAAACCCTTGGGTGAAGCAGAACGAGAGTTTATTT
TGAATTTGAAGAAAAAAGGAATGCAAAGACAGGGTTTTGAATATGATGGGAAAATCAATGCCTGGGATCTATATTACTA
CATGACTCAGACAGAGGAACTCAAGTATTCCATAGACCAAGAGTTCCTCAAGGAATACTTCCCAATTGAGGTGGTCACT
GAAGGCTTGCTGAACACCTACCAGGAGTTGTTGGGACTTTCATTTGAACAAATGACAGATGCTCATGTTTGGAACAAGA
GTGTTACACTTTATACTGTGAAGGATAAAGCTACAGGAGAAGTATTGGGACAGTTCTATTTGGACCTCTATCCAAGGGA
AGGAAAATACAATCATGCGGCCTGCTTCGGTCTCCAGCCTGGCTGCCTTCTGCCTGATGGAAGCCGGATGATGGCAGTG
GCTGCCCTCGTGGTGAACTTCTCACAGCCAGTGGCAGGTCGTCCCTCTCTCCTGAGACACGACGAGGTGAGGACTTACT
TTCATGAGTTTGGTCACGTGATGCATCAGATTTGTGCACAGACTGATTTTGCACGATTTAGCGGAACAAATGTGGAAAC
TGACTTTGTAGAGGTGCCATCGCAAATGCTTGAAAATTGGGTGTGGGACGTCGATTCCCTCCGAAGATTGTCAAAACAT
TATAAAGATGGAAGCCCTATTGCAGACGATCTGCTTGAAAAACTTGTTGCTTCGCTTATGTTATTAGGTCTTCTGACCC
TGCGCCAGATTGTTTTGAGCAAAGTTGATCAGTCTCTTCATACCAACACATCGCCGGATGCTGCAAGTGAATATGCCAA
ATACTGCTCAGAAATATTAGGAGTTGCAGCTACTCCAGGTACAAATATGCCAGCTACCTTTGGACATTTGGCAGGGGGA
TACGATGGCCAATATTATGGATATCTTTGGAGTGAAGTATTTTCCATGGATATGTTTTACAGCTGTTTTAAAAAAGAAG
GGATAATGAATCCAGAGGTAGTTGGAATGAAATACAGAAACCTAATCCTGAAACCTGGGGGATCTCTGGACGGCATGGA
CATGCTCCACAATTTCTTGAAACGTGAGCCAAACCAAAAAGCGTTCCTAATGAGTAGAGGCCTGCATGCTCCGTGAACT
GGGGATCTTTGGTAGCCGTCCATGTCTGGAGGACAAG

A disclosed NOV6f polypeptide (SEQ ID NO:22) encoded by SEQ ID NO:21 is presented using the one-letter amino acid code in Table 6O. NOV6f amino acid changes, if any, are underlined in Table 6O.

TABLE 60
Encoded NOV6f protein sequence.

(SEQ ID NO:22)

MIARCLLAVRSLRRVGGSRILLRMTLGREVMSPLQAMSSYTVAGRNVLRWDLSPEQIKTRTEELIVQTKQVYDAVGMLGIEEVTY
ENCLQALADVEVKYIVERTMLDFPQHVSSDKEVRAASTEADKRLSRFDIEMSMRGDIFERIVHLQETCDLGKIKPEARRYLEKSI
KMGKRNGLHLPEQVQNEIKSMKKRMSELCIDFNKNLNEDDTFLVFSKAELGALPDDFIDSLEKTDDDKYKITLKYPHYFPVMKKC
CIPETRRRMEMAFNTRCKEENTIILQQLLPLRTKVAKLLGYSTHADFVLEMNTAKSTSRVTAFLDDLSQKLKPLGEAEREFILNL
KKKECKDRGFEYDGKINAWDLYYYMTQTEELKYSIDQEFLKEYFPIEVVTEGLLNTYQELLGLSFEQMTDAHVWNKSVTLYTVKD
KATGEVLGQFYLDLYPREGKYNHAACFGLQPGCLLPDGSRMMAVAALVVNFSQPVAGRPSLLRHDEVRTYFHEFGHVMHQICAQT
DFARFSGTNVETDFVEVPSQMLENWVWDVDSLRRLSKHYKDGSPIADDLLEKLVASLMLLGLLTLRQIVLSKVDQSLHTNTSPDA
ASEYAKYCSEILGVAATPGTNMPATFGHLAGGYDGQYYGYLWSEVFSMDMFYSCFKKEGIMNPEVVGMKYRNLILKPGGSLDGMD
MLHNFLKREPNQKAFLMSRGLHAP

A disclosed NOV6g nucleic acid (also referred to as c99.459) is a variant of NOV6a, encodes a novel neurolysin precursor-like protein, and is shown in Table 6P. NOV6g nucleotide changes are underlined in Table 6P.

TABLE 6P
NOV6g Nucleotide Sequence

(SEQ ID NO:23)

CCTCTCAGCGCTCCCATGATCGCCCGGTGCCTTTTGGCTGTGCGAAGCCTCCGCAGGGTTGGTGGTTCCAGGATTTTAC
TCAGAATGACGTTAGGAAGAGAAGTGATGTCTCCTCTTCAGGCAATGTCTTCCTATACTGTGGCTGGCAGAAATGTTTT
AAGATGGGATCTTTCACCAGAGCAAATTAAAACAAGAACTGAGGAGCTCATTGTGCAGACCAAACAGGTGTACGATGCT
GTTGGAATGCTCGGTATTGAGGAAGTAACTTACGAGAACTGTCTGCAGGCACTGGCAGATGTAGAAGTAAAGTATATAG
TGGAAAGGACCATGCTAGACTTTCCCCAGCATGTATCCTCTGACAAAGAAGTACGAGCAGCAAGTACAGAAGCAGACAA
AAGACTTTCTCGTTTTGATATTGAGATGAGCATGAGAGGAGATATATTTGAGAGAATTGTTCATTTACAGGAAACCTGT
GATCTGGGGAAGATAAAACCTGAGGCCAGACGATACTTGGAAAAGTCAATTAAAATGGGGAAAAGAAATGGGCTCCATC
TTCCTGAACAAGTACAGAATGAAATCAAATCAATGAAGAAAAGAATGAGTGAGCTATGTATTGATTTTAACAAAAACCT
CAATGAGGATGATACCTTCCTTGTATTTTCCAAGGCTGAACTTGGTGCTCTTCCTGATGATTTCATTGACAGTTTAGAA
AAGACAGATGATGACAAGTATAAAATTACCTTAAAATATCCACACTATTTCCCTGTCATGAAGAAATGTTGTATCCCTG
AAACCAGAAGAAGGATGGAAATGGCTTTTAATACAAGGTGCAAAGAGGAAAACACCATAATTTTGCAGCAGCTACTCCC
ACTGCGAACCAAGGTGGCCAAACTACTCGGTTATAGCACACATGCTGACTTCGTCCTTGAAATGAACACTGCAAAGAGC
ACAAGCCGCGTAACAGCCTTTCTAGATGATTTAAGCCAGAAGTTAAAACCCTTGGGTGAAGCAGAACGAGAGTTTATTT
TGAATTTGAAGAAAAAGGAATGCAAAGACAGGGGTTTTGAATATGATGGGAAAATCAATGCCTGGGATCTATATTACTA
CATGACTCAGACAGAGGAACTCAAGTATTCCATAGACCAAGAGTTCCTCAAGGAATACTTCCCAATTGAGGTGGTCACT
GAAGGCTTGCTGAACACCTACCAGGAGTTGTTGGGACTTTCATTTGAACAAATGACAGATGCTCATGTTTGGAACAAGA
GTGTTACACTTTATACTGTGAAGGATAAAGCTACAGGAGAAGTATTGGGACAGTTCTATTTGGACCTCTATCCAAGGGA
AGGAAAATACAATCATGCGGCCTGCTTCGGTCTCCAGCCTGGCTGCCTTCTGCCTGATGGAAGCCGGATGATGGCAGTG
GCTGCCCTCGTGGTGAACTTCTCACAGCCAGTGGCAGGTCGTCCCTCTCTCCTGAGACACGACGAGGTGAGGACTTACT
TTCATGAGTTTGGTCACGTGATGCATCAGATTTGTGCACAGACTGATTTTGCACGATTTAGCGGAACAAATGTGGAAAC
TGACTTTGTAGAGGTGCCATCGCAAATGCTTGAAAATTGGGTGTGGGACGTCGATTCCCTCCGAAGATTGTCAAAACAT
TATAAAGATGGAAGCCCTATTGCAGACGATCTGCTTGAAAAACTTGTTGCTTCGCTTATGTTATTAGGTCTTCTGACCC
TGCGCCAGATTGTTTTGAGCAAAGTTGATCAGTCTCTTCATACCAACACATCGCTGGATGCCGCAAGTGATTATGCCAA
ATACTGCTCAGAAATATTAGGAGTTGCAGCTACTCCAGGTACAAATATGCCAGCTACCTTTGGACATTTGGCAGGGGGA
TACGATGGCCAATATTATGGATATCTTTGGAGTGAAGTATTTTCCATGGATATGTTTTACAGCTGTTTTAAAAAAGAAG
GGATAATGAATCCAGAGGTAGTTGGAATGAAATACAGAAACCTAATCCTGAAACCTGGGGGATCTCTGGACGGCATGGA
CATGCTCCACAATTTCTTGAAACGTGAGCCAAACCAAAAAGCGTTCCTAATGAGTAGAGGCCTGCATGCTCCGTGAACT
GGGGATCTTTGGTAGCCGTCCATGTCTGGAGGACAAG
GGGGATCTTTGGTAGCCGTCCATGTCTGGAGGACATG

A disclosed NOV6g polypeptide (SEQ ID NO:24) encoded by SEQ ID NO:23 is presented using the one-letter amino acid code in Table 6Q. NOV6g amino acid changes, if any, are underlined in Table 6Q.

TABLE 6Q
Encoded NOV6g protein sequence.

(SEQ ID NO:24)

EEVTYENCLQALADVEVKYIVERTMLDFPQHVSSDKEVRAASTEADKRLSRFDIEMSMRGDIFERIVHLQETCDLGKIKPEARRY
LEKSIKMGKRNGLHLPEQVQNEIKSMKKRMSELCIDFNKNLNEDDTFLVFSKAELGALPDDFIDSLEKTDDDKYKITLKYPHYFP
VMKKCCIPETRRRMEMAFNTRCKEENTIILQQLLPLRTKVAKLLGYSTHADFVLEMNTAKSTSRVTAFLDDLSQKLKPLGEAERE
FILNLKKKECKDRGFEYDGKINAWDLYYYMTQTEELKYSIDQEFLKEYFPIEVVTEGLLNTYQELLGLSFEQMTDAHVWNKSVTL
YTVKDKATGEVLGQFYLDLYPREGKYNHAACFGLQPGCLLPDGSRMMAVAALVVNFSQPVAGRPSLLRHDEVRTYFHEFGHVMHQ
ICAQTDFARFSGTNVETDFVEVPSQMLENWVWDVDSLRRLSKHYKDGSPIADDLLEKLVASLMLLGLLTLRQIVLSKVDQSLHTN
TSLDAASEYAKYCSEILGVAATPGTNMPATFGHLAGGYDGQYYGYLWSEVFSMDMFYSCFKKEGIMNPEVVGMKYRNLILKPGGS
LDGMDMLHNFLKREPNQKAFLMSRGLHAP

A disclosed NOV6h nucleic acid (also referred to as c99.460) is a variant of NOV6a, encodes a novel neurolysin precursor-like protein, and is shown in Table 6R. NOV6h nucleotide changes are underlined in Table 6R.

TABLE 6R
NOV6h Nucleotide Sequence

(SEQ ID NO:25)

CCTCTCAGCGCTCCCATGATCGCCCGGTGCCTTTTGGCTGTGCGAAGCCTCCGCAGGGTTGGTGGTTCCAGGATTTTAC
TCAGAATCACGTTAGGAAGAGAAGTGATGTCTCCTCTTCAGGCAATGTCTTCCTATACTGTGGCTGGCAGAAATGTTTT
AAGATGGGATCTTTCACCAGAGCAAATTAAAACAAGAACTGAGGAGCTCATTGTGCAGACCAAACAGGTGTACGATGCT
GTTGGAATGCTCGGTATTGAGGAAGTAACTTACGAGAACTGTCTGCAGGCACTGGCAGATGTAGAAGTAAAGTATATAG
TGGAAAGGACCATGCTAGACTTTCCCCAGCATGTATCCTCTGACAAAGAAGTACGAGCAGCAAGTACAGAAGCAGACAA
AAGACTTTCTCGTTTTGATATTGAGATGAGCATGAGAGGAGATATATTTGAGAGAATTGTTCATTTACAGGAAACCTGT
GATCTGGGGAAGATAAAACCTGAGGCCAGACGATACTTGGAAAAGTCAATTAAAATGGGGAAAAGAAATGGGCTCCATC
TTCCTGAACAAGTACAGAATGAAATCAAATCAATGAAGAAAAGAATGAGTGAGCTATGTATTGATTTTAACAAAAACCT
CAATGAGGATGATACCTTCCTTGTATTTTCCAAGGCTGAACTTGGTGCTCTTCCTGATGATTTCATTGACAGTTTAGAA
AAGACAGATGATGACAAGTATAAAATTACCTTAAAATATCCACACTATTTCCCTGTCATGAAGAAATGTTGTATCCCTG
AAACCAGAAGAAGGATGGAAATGGCTTTTAATACAAGGTGCAAAGAGGAAAACACCATAATTTTGCAGCAGCTACTCCC
ACTGCGAACCAAGGTGGCCAAACTACTCGGTTATAGCACACATGCTGACTTCGTCCTTGAAATGAACACTGCAAAGAGC
ACAAGCCGCGTAACAGCCTTTCTAGATGATTTAAGCCAGAAGTTAAAACCCTTGGGTGAAGCAGAACGAGAGTTTATTT
TGAATTTGAAGAAAAAGGAATGCAAAGACAGGGGTTTTGAATATGATGGGAAAATCAATGCCTGGGATCTATATTACTA
CATGACTCAGACAGAGGAACTCAAGTATTCCATAGACCAAGAGTTCCTCAAGGAATACTTCCCAATTGAGGTGGTCACT
GAAGGCTTGCTGAACACCTACCAGGAGTTGTTGGGACTTTCATTTGAACAAATGACAGATGCTCATGTTTGGAACAAGA
GTGTTACACTTTATACTGTGAAGGATAAAGCTACAGGAGAAGTATTGGGACAGTTCTATTTGGACCTCTATCCAAGGGA
AGGAAAATACAATCATGCGGCCTGCTTCGGTCTCCAGCCTGGCTGCCTTCTGCCTGATGGAAGCCGGATGATGGCAGTG
GCTGCCCTCGTGGTGAACTTCTCACAGCCAGTGGCAGGTCGTCCCTCTCTCCTGAGACACGACGAGGTGAGGACTTACT
TTCATGAGTTTGGTCACGTGATGCATCAGATTTGTGCACAGACTGATTTTGCACGATTTAGCGGAACAAATGTGGAAAC
TGACTTTGTAGAGGTGCCATCGCAAATGCTTGAAAATTGGGTGTGGGACGTCGATTCCCTCCGAAGATTGTCAAAACAT
TATAAAGATGGAAGCCCTATTGCAGACGATCTGCTTGAAAAACTTGTTGCTTCGCTTATGTTATTAGGTCTTCTGACCC
TGCGCCAGATTGTTTTGAGCAAAGTTGATCAGTCTCTTCATACCAACACATCGCTGGATGCTGCAAGTGAATATGCTAA
ATACTGCTCAGAAATATTAGGAGTGGCAGCTACTCCAGGTACAAATATGCCAGCTACCTTTGGACATTTGGCAGGGGGA
TACGATGGCCAATATTATGGATATCTTTGGAGTGAAGTATTTTCCATGGATATGTTTTACAGCTGTTTTAAAAAAGAAG
GGATAATGAATCCAGAGGTAGTTGGAATGAAATACAGAAACCTAATCCTGAAACCTGGGGGATCTCTGGACGGCATGGA
CATGCTCCACAATTTCTTGAAACGTGAGCCAAACCAAAAAGCGTTCCTAATGAGTAGAGGCCTGCATGCTCCGTGAACT
GGGGATCTTTGGTAGCCGTCCATGTCTGGAGGACAAG

A disclosed NOV6h polypeptide (SEQ ID NO:26) encoded by SEQ ID NO:25 is presented using the one-letter amino acid code in Table 6S. NOV6h amino acid changes, if any, are underlined in Table 6S.

TABLE 65
Encoded NOV6h protein sequence.

(SEQ ID NO:26)

MIARCLLAVRSLRRVGGSRILLRMTLGREVMSPLQAMSSYTVAGRNVLRWDLSPEQIKTRTEELIVQTKQVYDAVGMLGIEEVTY
ENCLQALADVEVKYIVERTMLDFPQHVSSDKEVRAASTEADKRLSRFDIEMSMRGDIFERIVHLQETCDLGKIKPEARRYLEKSI
KMGKRNGLHLPEQVQNEIKSMKKRMSELCIDFNKNLNEDDTFLVFSKAELGALPDDFIDSLEKTDDDKYKITLKYPHYFPVMKKC
CIPETRRRMEMAFNTRCKEENTIILQQLLPLRTKVAKLLGYSTHADFVLEMNTAKSTSRVTAFLDDLSQKLKPLGEAEREFILNL
KKKECKDRGFEYDGKINAWDLYYYMTQTEELKYSIDQEFLKEYFPIEVVTEGLLNTYQELLGLSFEQMTDAHVWNKSVTLYTVKD
KATGEVLGQFYLDLYPREGKYNHAACFGLQPGCLLPDGSRMMAVAALVVNFSQPVAGRPSLLRHDEVRTYFHEFGHVMHQICAQT
DFARFSGTNVETDFVEVPSQMLENWVWDVDSLRRLSKHYKDGSPIADDLLEKLVASLMLLGLLTLRQIVLSKVDQSLHTNTSLDA
ASEYAKYCSEILGVAATPGTNMPATFGHLAGGYDGQYYGYLWSEVFSMDMFYSCFKKEGIMNPEVVGMKYRNLILKPGGSLDGMD
MLHNFLKREPNQKAFLMSRGLHAP

A disclosed NOV6i nucleic acid (also referred to as c99.752) is a variant of NOV6a, encodes a novel neurolysin precursor-like protein, and is shown in Table 6T. NOV6i nucleotide changes are underlined in Table 6T.

TABLE 6T
NOV6Ti Nucleotide Sequence

(SEQ ID NO:27)

CCTCTCAGCGCTCCCATGATCGCCCGGTGCCTTTTGGCTGTGCGAAGCCTCCGCAGGGTTGGTGGTTCCAGGATTTTAC
TCAGAATGACGTTAGGAAGAGAAGTGATGTCTCCTCTTCAGGCAATGTCTTCCTATACTGTGGCTGGCAGAAATGTTTT
AAGATGGGATCTTTCACCAGAGCAAATTAAAACAAGAACTGAGGAGCTCATTGTGCAGACCAAACAGGTGTACGATGCT
GTTGGAATGCTCGGTATTGAGGAAGTAACTTACGAGAACTGTCTGCAGGCACTGGCAGATGTAGAAGTAAAGTATATAG
TGGAAAGGACCATGCTAGACTTTCCCCAGCATGTATCCTCTGACAAAGAAGTACGAGCAGCAAGTACAGAAGCAGACAA
AAGACTTTCTCGTTTTGATATTGAGATGAGCATGAGAGGAGATATATTTGAGAGAATTGTTCATTTACAGGAAACCTGT
GATCTGGGGAAGATAAAACCTGAGGCCAGACGATACTTGGAAAAGTCAATTAAAATGGGGAAAAGAAATGGGCTCCATC
TTCCTGAACAAGTACAGAATGAAATCAAATCAATGAAGAAAAGAATGAGTGAGCTATGTATTGATTTTAACAAAAACCT
CAATGAGGATGATACCTTCCTTGTATTTTCCAAGGCTGAACTTGGTGCTCTTCCTGATGATTTCATTGACAGTTTAGAA
AAGACAGATGATGACAAGTATAAAATTACCTTAAAATATCCACACTATTTCCCTGTCATGAAGAAATGTTGTATCCCTG
AAACCAGAAGAAGGATGGAAATGGCTTTTAATACAAGGTGCAAAGAGGAAAACACCATAATTTTGCAGCAGCTACTCCC
ACTGCGAACCAAGGTGGCCAAACTACTCGGTTATAGCACACATGCTGACTTCGTCCTTGAAATGAACACTGCAAAGAGC
ACAAGCCGCGTAACAGCCTTTCTAGATGATTTAAGCCAGAAGTTAAAACCCTTGGGTGAAGCAGAACGAGAGTTTATTT
TGAATTTGAAGAAAAAGGAATGCAAAGACAGGGGTTTTGAATATGATGGGAAAATCAATGCCTGGGATCTATATTACTA
CATGACTCAGACAGAGGAACTCAAGTATTCCATAGACCAAGAGTTCCTCAAGGAATACTTCCCAATTGAGGTGGTCACT
GAAGGCTTGCTGAACACCTACCAGGAGTTGTTGGGACTTTCATTTGAACAAATGACAGATGCTCATGTTTGGAACAAGA
GTGTTACACTTTATACTGTGAAGGATAAAGCTACAGGAGAAGTATTGGGACAGTTCTATTTGGACCTCTATCCAAGGGA
AGGAAAATACAATCATGCGGCCTGCTTCGGTCTCCAGCCTGGCTGCCTTCTGCCTGATGGAAGCCGGATGATGGCAGTG
GCTGCCCTCGTGGTGAACTTCTCACAGCCAGTGGCAGGTCGTCCCTCTCTCCTGAGACACGACGAGGTGAGGACTTACT
TTCATGAGTTTGGTCACGTGATGCATCAGATTTGTGCACAGACTGATTTTGCACGATTTAGCGGAACAAATGTGGAAAC
TGACTTTGTAGAGGTGCCATCGCAAATGCTTGAAAATTGGGTGTGGGACGTCGATTCCCTCCGAAGATTGTCAAAACAT
TATAAAGATGGAAGCCCTATTGCAGACGATCTGCTTGAAAAACTTGTTGCTTCGCTTATGTTATTAGGTCTTCTGACCC
TGCGCCAGATTGTTTTGAGCAAAGTTGATCAGTCTCTTCATACCAACACATCGCTGGATGCTGCAAGTGAATATGCCAA
ATACTGCACAGAAATATTAGGAGTTGCAGCTACTCCAGGTACAAATATGCCAGCTACCTTTGGACATTTGGCAGGGGGA
TACGATGGCCAATATTATGGATATCTTTGGAGTGAAGTATTTTCCATGGATATGTTTTACAGCTGTTTTAAAAAAGAAG
GGATAATGAATCCAGAGGTAGTTGGAATGAAATACAGAAACCTAATCCTGAAACCTGGGGGATCTCTGGACGGCATGGA
CATGCTCCACAATTTCTTGAAACGTGAGCCAAACCAAAAAGCGTTCCTAATGAGTAGAGGCCTGCATGCTCCGTGAACT
GGGGATCTTTGGTAGCCGTCCATGTCTGGAGGACAAG

A disclosed NOV6i polypeptide (SEQ ID NO:28) encoded by SEQ ID NO:27 is presented using the one-letter amino acid code in Table 6U. NOV6i amino acid changes, if any, are underlined in Table 6U.

TABLE 6U
Encoded NOV6i protein sequence.

(SEQ ID NO:28)

MIARCLLAVRSLRRVGGSRILLRMTLGREVMSPLQAMSSYTVAGRNVLRWDLSPEQIKTRTEELIVQTKQVYDAVGMLGIEEVTY
ENCLQALADVEVKYIVERTMLDFPQHVSSDKEVRAASTEADKRLSRFDIEMSMRGDIFERIVHLQETCDLGKIKPEARRYLEKSI
KMGKRNGLHLPEQVQNEIKSMKKRMSELCIDFNKNLNEDDTFLVFSKAELGALPDDFIDSLEKTDDDKYKITLKYPHYFPVMKKC
CIPETRRRMEMAFNTRCKEENTIILQQLLPLRTKVAKLLGYSTHADFVLEMNTAKSTSRVTAFLDDLSQKLKPLGEAEREFILNL
KKKECKDRGFEYDGKINAWDLYYYMTQTEELKYSIDQEFLKEYFPIEVVTEGLLNTYQELLGLSFEQMTDAHVWNKSVTLYTVKD
KATGEVLGQFYLDLYPREGKYNHAACFGLQPGCLLPDGSRMMAVAALVVNFSQPVAGRPSLLRHDEVRTYFHEFGHVMHQICAQT
DFARFSGTNVETDFVEVPSQMLENWVWDVDSLRRLSKHYKDGSPIADDLLEKLVASLMLLGLLTLRQIVLSKVDQSLHTNTSLDA
ASEYAKYCTEILGVAATPGTNMPATFGHLAGGYDGQYYGYLWSEVFSMDMFYSCFKKEGIMNPEVVGMKYRNLILKPGGSLDGMD
MLHNFLKREPNQKAFLMSRGLHAP

Homologies to any of the above NOV6 proteins will be shared by the other NOV6 proteins insofar as they are homologous to each other as shown above. Any reference to NOV6 is assumed to refer to all three of the NOV6 proteins in general, unless otherwise noted.

A human genomic clone encompassing exons 1-3 of the neurotensin/nuromedin N gene was identified using a canine neurotensin complementary DNA probe. Sequence comparisons revealed that the 120-amino acid portion of the precursor sequence encoded by exons 1-3 is 89% identical to previously determined cow and dog sequences and that the proximal 250 bp of 5′ flanking sequences are strikingly conserved between rat and human. The 5′ flanking sequence contains cis-regulatory sites required for the induction of neurotensin/neuromedin N gene expression in PC12 cells, including AP1 sites and two cyclic adenosine-5′-monophosphate response elements. Oligonucleotide probes based on the human sequence were used to examine the distribution of neurotensin/neuromedin N messenger RNA in the ventral mesencephalon of schizophrenics and age- and sex-matched controls. Neurotensin/neuromedin N messenger RNA was observed in ventral mesencephalic cells some of which also contained melanin pigment or tyrosine hydroxylase messenger RNA. Neurons expressing neurotensin/neuromedin N messenger RNA were observed in the ventral mesencephalon of both schizophrenic and non-schizophrenic humans. PMID: 1436492, UI: 93063858

Neurotensin is a small neuropeptide of 13 amino acids that may function as a neurotransmitter or neuromodulator in the central nervous system. In the CNS, neurotensin is localized to the catecholamine-containing neurons. A catecholamine-producing cell line can also produce NT. Lithium salts, widely used in the treatment of manic-depressive patients, dramatically potentiate NT gene expression in this cell line. Gerhard et al. (1989) used a canine cDNA as a probe on a somatic cell hybrid panel to determine that the human gene is located on chromosome 12.

The tridecapeptide neurotensin (162650) is widely distributed in various regions of the brain and in peripheral tissues. In the brain, neurotensin acts as a neuromodulator, in particular of dopamine transmission in the nigrostriatal and mesocorticolimbic systems, suggesting its possible implication in dopamine-associated behavioral neurodegenerative and neuropsychiatric disorders. Its various effects are mediated by specific membrane receptors. Vita et al. (1993) isolated a cDNA encoding the human neurotensin receptor and showed that it predicts a 418-amino acid protein that shares 84% homology with the rat protein. Le et al. (1997) also cloned the human neurotensin receptor (NTR) cDNA and its genomic DNA. The gene is encoded by 4 exons spanning more than 10 kb. The authors identified a highly polymorphic tetranucleotide repeat approximately 3 kb from the gene. Southern blot analysis revealed that the NTR gene is present in the human genome as a single-copy gene. Le et al. (1997) stated that-the neurotensin receptor has 7 transmembrane spanning regions and high homology to other receptors that couple to G proteins.

The above defined information for NOV6 suggests that NOV6 may function as a member of a Neurolysin family. Therefore, the NOV6 nucleic acids and proteins of the invention are useful in potential therapeutic applications implicated in various diseases and disorders described below and/or other pathologies. For example, the NOV6 compositions of the present invention will have efficacy for treatment of patients suffering from behavioral neurodegenerative and neuropsychiatric disorders such as schizophrenia, anxiety disorders, bipolar disorders, depression, eating disorders, personality disorders, or sleeping disorders, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Transplantation, Adrenoleukodystrophy, Congenital Adrenal Hyperplasia, Diabetes, Von Hippel-Lindau (VHL) syndrome, Pancreatitis, Endometriosis, Fertility, Inflammatory bowel disease, Diverticular disease, Hirschsprung's disease, Crohn's Disease, Hemophilia, hypercoagulation, Idiopathic thrombocytopenic purpura, immunodeficiencies, Osteoporosis, Hypercalceimia, Arthritis, Ankylosing spondylitis, Scoliosis, Endocrine dysfunctions, Diabetes, Growth and reproductive disorders, Psoriasis, Actinic keratosis, Acne, Hair growth, allopecia, pigmentation disorders and endocrine disorders. The NOV6 nucleic acid encoding neurolysin precursor-like protein, and the neurolysin precursor-like protein of the invention, or fragments thereof, may further be useful in diagnostic applications, wherein the presence or amount of the nucleic acid or the protein are to be assessed.

NOV7

NOV7 includes six novel gamma-aminobutyric acid (GABA) transporter-like receptor proteins disclosed below. The disclosed proteins have been named NOV7a, NOV7b, NOV7c, NOV7d, NOV7e and NOV7f.

NOV7a

A disclosed NOV7a nucleic acid of 1763 nucleotides (also referred to ba122o1) encoding a novel GABA transporter-like receptor protein is shown in Table 7A. An open reading frame was identified beginning with an ATG initiation codon at nucleotides 141-143 and ending with a TAG codon at nucleotides 1716-1719. Putative untranslated regions, if any, are found upstream from the initiation codon and downstream from the termination codon in Table 7A, and the start and stop codons are in bold letters.

TABLE 7A
NOV7a Nucleotide Sequence

(SEQ ID NO:29)

TCATGAGCCAGAGAGCCCCGGGGCGCCGCGCGGAGAGCAAGCGGAGATAGCGACTTTGCGCCCCCCAGCC
CTCGCCTTCTTGCATCGCGTTCCCCGCATCCTCGGGTCCTTCTGTCCTTTCCGCTGTCCCCACCGCCGCC
ATGGCCACCTTGCTCCGCAGCAAGCTGTCCAACGTGGCCACGTCCGTGTCCAACAAGTCCCAGGCCAAGA
TGAGCGGCATGTTCGCCAGGATGGGTTTTCAGGCGGCCACGGATGAGGAGGCGGTGGGCTTCGCGCATTG
CGACGACCTCGACTTTGAGCACCGCCAGGGCCTGCAGATGGACATCCTGAAAGCCGAGGGAGAGCCCTGC
GGGGACGAGGGCGCTGAAGCGCCCGTCGAGGGAGACATCCATTATCAGCGAGGCGGCGGAGCTCCTCTGC
CGCCCTCCGGCTCCAAGGACCAGGTGGGAGGTGGTGGCGAATTCGGGGGCCACGACAAGCCCAAAATCAC
GGCGTGGGAGGCAGGCTGGAACGTGACCAACGCCATCCAGGGCATGTTCGTGCTGGGCCTACCCTACGCC
ATCCTGCACGGCGGCTACCTGGGGTTGTTTCTCATCATCTTCGCCGCCGTTGTGTGCTGCTACACCGGCA
AGATCCTCATCGCGTGCCTGTACGAGGAGAATGAAGACGGCGAGGTGGTGCGCGTGCGGGACTCGTACGT
GGCCATAGCCAACGCCTGCTGCGCCCCGCGCTTCCCAACGCTGGGCGGCCGAGTGGTGAACGTAGCGCAG
ATCATCGAGCTGGTGATGACGTGCATCCTGTACGTGGTGGTGAGTGGCAACCTCATGTACAACAGCTTCC
CGGGGCTGCCCGTGTCGCAGAAGTCCTGGTCCATTATCGCCACGGCCGTGCTGCTGCCTTGCGCCTTCCT
TAAGAACCTCAAGGCCGTGTCCAAGTTCAGTCTGCTGTGCACTCTGGCCCACTTCGTCATCAATATCCTG
GTCATAGCCTACTGTCTATCGCGGGCGCGCGACTGGGCCTGGGAGAAGGTCAAGTTCTACATCGACGTCA
AGAAGTTCCCCATCTCCATTGGCATCATCGTGTTCAGCTACACGTCTCAGATCTTCCTGCCTTCGCTGGA
GGGCAATATGCAGCAGCCCAGCGAGTTCCACTGCATGATGAACTGGACGCACATCGCAGCCTGCGTGCTC
AAGGGCCTCTTCGCGCTCGTCGCCTACCTCACCTGGGCCGACGAGACCAAGGAGGTCATCACGGATAACC
TGCCCGGCTCCATCCGCGCCGTGGTCAACATCTTTCTGGTGGCCAAGGCGCTGTTGTCCTATCCTCTGCC
ATTCTTTGCCGCTGTCGAGGTGCTGGAGAAGTCGCTCTTCCAGGAAGGCAGCCGCGCCTTTTTCCCGGCC
TGCTACAGCGGCGACGGGCGCCTGAAGTCCTGGGGGCTGACGCTGCGCTGCGCGCTCGTCGTCTTCACGC
TGCTCATGGCCATTTATGTGCCGCAGTTCGCGCTGCTCATGGGCCTCACCGGCAGCCTCACGGGCGCCGG
CCTCTGTTTCTTGCTGCCCAGCCTCTTTCACCTGCGCCTGCTCTGGCGCAAGCTGCTGTGGCACCAAGTC
TTCTTCGACGTCGCCATCTTCGTCATCGGCGGCATCTGCAGCGTGTCCGGCTTCGTGCACTCCCTCGAGG
GCCTCATCGAAGCCTACCGAACCAACGCGGAGGACTAGGGCGCAAGGGCGAGCCCCCGCCGCGCTTCTGC
GCTCTCTCCCTTC

The disclosed NOV7a nucleic acid sequence, localized to chromosome 20, has 1532 of 1695 bases (90%) identical to a Homo sapiens vesicular GABA transporter (VGAT) mRNA (gb: acc: AF030253) (E=4.3e⁻³⁰⁸).

A disclosed NOV7a polypeptide (SEQ ID NO:30) encoded by SEQ ID NO:29 is 525 amino acid residues and is presented using the one-letter amino acid code in Table 7B. Signal P, Psort and/or Hydropathy results predict that NOV7a does not contain a signal peptide and is likely to be localized in the plasma membrane with a certainty of 0.6000.

TABLE 7B
Encoded NOV7a protein sequence.

(SEQ ID NO:30)

MATLLRSKLSNVATSVSNKSQAKMSGMFARMGFQAATDEEAVGFAHCDDLDFEHRQGLQMDILKAEGEPC
GDEGAEAPVEGDIHYQRGGGAPLPPSGSKDQVGGGGEFGGHDKPKITAWEAGWNVTNAIQGMFVLGLPYA
ILHGGYLGLFLIIFAAVVCCYTGKILIACLYEENEDGEVVRVRDSYVAIANACCAPRFPTLGGRVVNVAQ
IIELVMTCILYVVVSGNLMYNSFPGLPVSQKSWSIIATAVLLPCAFLKNLKAVSKFSLLCTLAHFVINIL
VIAYCLSRARDWAWEKVKFYIDVKKFPISIGIIVFSYTSQIFLPSLEGNMQQPSEFHCMMNWTHIAACVL
KGLFALVAYLTWADETKEVITDNLPGSIRAVVNIFLVAKALLSYPLPFFAAVEVLEKSLFQEGSRAFFPA
CYSGDGRLKSWGLTLRCALVVFTLLMAIYVPHFALLMGLTGSLTGAGLCFLLPSLFHLRLLWRKLLWHQV
FFDVAIFVIGGICSVSGFVHSLEGLIEAYRTNAED

The NOV7a amino acid sequence has 518 of 525 amino acid residues (98%) identical to, and 519 of 525 amino acid residues (98%) similar to the Homo sapiens 525 amino acid residue vesicular GABA transporter protein (SPTREMBL-ACC: 035458) (E=0.0).

NOV7a is expressed in at least the following tissues/cell lines: Brain, HS-528T/MCF-7, BT549/MDA-MB-231, OVCAR-3/OVCAR-4, IGROV-1, OVCAR-8, SK-OV-3 & OVCAR-5.

Novel variants for the NOV7a nucleic acid and vesicular GABA transporter-like protein are also disclosed herein as variants of NOV7a. Variants, as described above, are reported individually, but any combination of all or a subset are also included.

A disclosed NOV7b nucleic acid (also referred to as 13374575) is a variant of NOV7a, encodes a novel vesicular GABA transporter-like protein, and is shown in Table 7C. NOV7b nucleotide changes are underlined in Table 7C.

TABLE 7C
NOV7b Nucleotide Sequence

(SEQ ID NO:31)

GCGGAGAGCAAGCGGAGATAGCGACTTTGCGCCCCCCAGCCCTCGCCTTCTTGCATCGCGTTCCCCGCATCCTCGGGTC
CTTCTGTCCTTTCCGCTGTCCCCACCGCCGCCATGGCCACCTTGCTCCGCAGCAAGCTGTCCAACGTGGCCACGTCCGT
GTCCAACAAGTCCCAGGCCAAGATGAGCGGCATGTTCGCCAGGATGGGTTTTCAGGCGGCCACGGATGAGGAGGCGGTG
GGCTTCGCGCATTGCGACGACCTCGACTTTGAGCACCGCCAGGGCCTGCAGATGGACATCCTGAAAGCCGAGGGAGAGC
CCTGCGGGGACGAGGGCGCTGAAGCGCCCGTCGAGGGAGACATCCATTATCAGCGAGGCAGCGGAGCTCCTCTGCCGCC
CTCCGGCTCCAAGGACCAGGTGGGAGGTGGTGGCGAATTCGGGGGCCACGACAAGCCCAAAATCACGGCGTGGGAGGCA
GGCTGGAACGTGACCAACGCCATCCAGGGCATGTTCGTGCTGGGCCTACCCTACGCCATCCTGCACGGCGGCTACCTGG
GGTTGTTTCTCATCATCTTCGCCGCCGTTGTGTGCTGCTACACCGGCAAGATCCTCATCGCGTGCCTGTACGAGGAGAA
TGAAGACGGCGAGGTGGTGCGCGTGCGGGACTCGTACGTGGCCATAGCCAACGCCTGCTGCGCCCCGCGCTTCCCAACG
CTGGGCGGCCGAGTGGTGAACGTAGCGCAGATCATCGAGCTGGTGATGACGTGCATCCTGTACGTGGTGGTGAGTGGCA
ACCTCATGTACAACAGCTTCCCGGGGCTGCCCGTGTCGCAGAAGTCCTGGTCCATTATCGCCACGGCCGTGCTGCTGCC
TTGCGCCTTCCTTAAGAACCTCAAGGCCGTGTCCAAGTTCAGTCTGCTGTGCACTCTGGCCCACTTCGTCATCAATATC
CTGGTCATAGCCTACTGTCTATCGCGGGCGCGCGACTGGGCCTGGGAGAAGGTCAAGTTCTACATCGACGTCAAGAAGT
TCCCCATCTCCATTGGCATCATCGTGTTCAGCTACACGTCTCAGATCTTCCTGCCTTCGCTGGAGGGCAATATGCAGCA
GCCCAGCGAGTTCCACTGCATGATGAACTGGACGCACATCGCAGCCTGCGTGCTCAAGGGCCTCTTCGCGCTCGTCGCC
TACCTCACCTGGGCCGACGAGACCAAGGAGGCCATCACGGATAACCTGCCCGGCTCCATCCGCGCCGTGGTCAACATCT
TTCCGGTGGCCAAGGCGCTGTTGTCCTATCCTCTGCCATTCTTTGCCGCTGTCGAGGTGCTGGAGAAGTCGCTCTTCCA
GGAAGGCAGCCGCGCCTTTTTCCCGGCCTGCTACAGCGGCGACGGGCGCCTGAAGTCCTGGGGGCTGACGCTGCGCTGC
GCGCTCGTCGTCTTCACGCTGCTCATGGCCATTTATGTGCCGCACTTCGCGCTGCTCATGGGCCTCACCGGCAGCCTCA
CGGGCGCCGGCCTCTGTTTCTTGCTGCCCAGCCTCTTTCACCTGCGCCTGCTCTGGCGCAAGCTGCTGTGGCACCAAGT
CTTCTTCGACGTCGCCATCTTCGTCATCGGCGGCATCTGCAGCGTGTCCGGCTTCGTGCACTCCCTCGAGGGCCTCATC
GAAGCCTACCGAACCAACGCGGAGGACTAGGGCGCAAGGGCGAGCCCCCGCCGCGCTTCTGCGCTCTCTCCCTTC

A disclosed NOV7b polypeptide (SEQ ID NO:32) encoded by SEQ ID NO:31 is is presented using the one-letter amino acid code in Table 7D. NOV7b amino acid changes, if any, are underlined in Table 7D.

TABLE 7D
Encoded NOV7b protein sequence.

(SEQ ID NO:32)

MATLLRSKLSNVATSVSNKSQAKMSGMFARMGFQAATDEEAVGFAHCDDLDFEHRQGLQMDILKAEGEPCGDEGAEAPVEGDIHY
QRGSGAPLPPSGSKDQVGGGGEFGGHDKPKITAWEAGWNVTNAIQGMFVLGLPYAILHGGYLGLFLIIFAAVVCCYTGKILIACL
YEENEDGEVVRVRDSYVAIANACCAPRFPTLGGRVVNVAQIIELVMTCILYVVVSGNLMYNSFPGLPVSQKSWSIIATAVLLPCA
FLKNLKAVSKFSLLCTLAHFVINILVIAYCLSRARDWAWEKVKFYIDVKKFPISIGIIVFSYTSQIFLPSLEGNMQQPSEFHCMM
NWTHIAACVLKGLFALVAYLTWADETKEAITDNLPGSIRAVVNIFPVAKALLSYPLPFFAAVEVLEKSLFQEGSRAFFPACYSGD
GRLKSWGLTLRCALVVFTLLMAIYVPHFALLMGLTGSLTGAGLCFLLPSLFHLRLLWRKLLWHQVFFDVAIFVIGGICSVSGFVH
SLEGLIEAYRTNAED

A disclosed NOV7c nucleic acid (also referred to as 13374576) is a variant of NOV7a, encodes a novel vesicular GABA transporter-like protein, and is shown in Table 7E. NOV7c nucleotide changes are underlined in Table 7E.

TABLE 7E
NOV7c Nucleotide Sequence

(SEQ ID NO:33)

GCGGAGAGCAAGCCGGAGATAGCGACTTTGCGCCCCCCAGCCCTCGCCTTCTTGCATCGCGTTCCCCGOATCCTCGGGTC
CTTCTGTCCTTTCCGCTGTCCCCACCGCCGCCATGGCCACCTTGCTCCGCAGCAAGCTGTCCAACGTGGCCACGTCCGT
GTCCAACAAGTCCCAGGCCAAGATGAGCGGCATGTTCGCCAGGATGGGTTTTCAGGCGGCCACGGATGAGGAGGCGGTG
GGCTTCGCGCATTGCGACGACCTCGACTTTGAGCACCGCCAGGGCCTGCAGATGGACATCCTGAAAGCCGAGGGAGAGC
CCTGCCGGGACCAGGGCGCTGAGCGCCCGTCGAGGGAGACATCCATTATCAGCGAGGCAGCGGAGCTCCTCTGCCGCC
CTCCGGCTCCAAGGACCAGGTGGGAGGTGGTGGCGAATTCGGGGGCCACGACAAGCCCAAAATCACGGCGTGGGAGGCA
GGCTGGACGTGACCAACGCCATCCAGGGCATGTTCGTGCTGGGCCTACCCTACGCCATCCTGCACGGCGGCTACCTGG
GGTTGTTTCPCATCATCTTCGCCGCCGTTGTGTGCTGCTACACCGGCAAGATCCTCATCGCGTGCCTGTACGAGGAGAA
TGAAGACGGCGAGGTGGTGCGCGTGCGGGACTCGTACGTGGCCATAGCCAACGCCTGCTGCGCCCCGCGCTTCCCAACG
CTGGGCGGCCGAGTGGTGAACGTAGCGCAGATCATCGAGCTGGTGATGACGTGCATCCTGTACGTGGTGGTGAGTGGCA
ACCTCATCTACAACAGCTTCCCGGGGCTGCCCGTGTCGCAGAAGTCCTGGTCCATTATCGCCACGGCCGTGCTGCTGCC
TPGCGCCTTCCTTAAACCTCAGCCGTGTCCAGTTCAGTCTGCTGTGCACTCTGGCCCACTTCGTCATCAATATC
CTGGTCATAGCCTACTGTCTATCGCGGGCGCGCGACTGGGCCTGGGAGAAGGTCAAGTTCTACATCGACGTCAAGAAGT
TCCCCATCTCCATTGGCATCATCGTGTTCAGCTACACGTCTCAGATCTTCCTGCCTTCGCTGGAGGGATATGCAGCA
GCCCAGCGAGTTCCACTGCATGATGAACTGGACGCACATCGCAGCCTGCGTGCTCAAGGGCCTCTTCGCGCTCGTCGCC
TACCTCACCGGGCCGACGAGACCAAGGAGGTCATCACGGATAACCTGCCCGGCTCCATCCGCGCCGTGGTCAACATCT
TTCTGGTGGCCAAGGCGCTGTTGTCCTATCCTCTGCCATTCTTTGCCGCTGTCGAGGTGCTGGAGAAGTCGCTCTTCCA
GGAGGCAGCCGCGCCTTTTTCCCGGCCTGCTACAGCCGCGACGGGCGCCGAAGTCCTGGGGGCTGACGCTGCGCTGC
GCGCTCGTCGTCTTCACGCTGCTCATGGCCATTTATGTGCCGCACTTCGCGCTGCTCATGGGCCTCACCGGCAGCCTCA
CGGGCGCCGGCCTCTGTTTCTTGCTGCCCAGCCTCTTTCACCTGCGCCTGCTCTGGCGCAAGCTGCTGTGGCACCAAGT
CTTCTTCGACGTCGCCATCTTCGTCATCGGCGGCATCTGCAGCGTGTCCGGCTTCGTGCACTCCCTCGAGGGCCTCATC
GAAGCCTACCGAACCAACGGGAGGACTAGGGCGCAAGGGCGAGCCCCCGCCGCGCTTCTGCGCTCTCTCCCTTC

A disclosed NOV7c polypeptide (SEQ ID NO:34) encoded by SEQ ID NO:33 is is presented using the one-letter amino acid code in Table 7F. NOV7c amino acid changes, if any, are underlined in Table 7F.

TABLE 7F
Encoded NOV7c protein sequence.

(SEQ ID NO:34)

MATLLRSKLSNVATSVSNKSQAKMSGFARMGFQAATDEEAVGFAHCDDLDFEHRQGLQMDILKAEGEPCGDEGAEAPVEGDIHY
QRGSGAPLPPSGSKDQVGGGGERGGHDKPKITAWEAGWNVTNAIQGMFVLGLPYAILHGCYLGLFLIIFAAVVCCYTGKILIACL
YEENEDGEVVRVRDSYVAIANACCAPRFPTLGGRVVNVAQIIELVMTCILYVVVSGNLMYNSPPGLPVSQKSWSIIATAVLLPCA
FLLKNAVSKFSLLCTLAHFVINILVIAYCLSRARDWAWEKVKFYIDVKKFPISIGIIVFSYTSQIFLPSLEGNMQQPSEFHCMM
NWTHIAACVLKGLFALVAYLTWADETKEVITDNLPGSIRAVVNIFLVAKALLSYPLPFFAAVEVLEKSLFQEGSRAFFPACYSGD
GRLKSWGLTLRCALVVFTLLMAIYVPHFALLMGLTGSLTGAGLCFLLPSLFHLRLLWRKLLWTIQVFFDVAIFVIGGICSVSGFVH
SLEGLIEAYRTNAED

A disclosed NOV7d nucleic acid (also referred to as 13374577) is a variant of NOV7a, encodes a novel vesicular GABA transporter-like protein, and is shown in Table 7G. NOV7d nucleotide changes are underlined in Table 7G.

TABLE 7G
NOV7d Nucleotide Sequence

(SEQ ID NO:35)

GCGGAGAGCAAGCGGAGATAGCGACTTTGCGCCCCCCAGCCCTCGCCTTCTTGCATCGCGTTCCCCGCATCCTCGGGTC
CTTCTGTCCTTTCCGCTGTCCCCACCGCCGCCATGGCCACCTTGCTCCGCAGCAAGCTGTCCAACGTGGCCACGTCCGT
GTCCAACAAGTCCCAGGCCAAGATGAGCGGCATGTTCGCCAGGATGGGTTTTCAGGCGGCCACGATGAGGAGGCGGTG
GGCTTCGCGCATTGCGACGACCTCGACTTTGAGCACCCCCAGGCCCTCCAGATCCACATCCTGAAAGCCGAGGGAGAGC
CCTGCGGGGACGAGGGCGCTGAAGCGCCCGTCGAGGGAGACATCCATTATCAGCGAGGCAGCGGAGCTCCTCTGCCGCC
CTCCGGCTCCAAGGACCAGGTGOGAGGTGGTCGCGAATTCGGGGGCCACCACAAGCCCAATCACGGCGTGGGAGGCA
GGCGGAACGTGACCAACGCCATCCAGGGCATGTTCGTGCTGGGCCTACCCTACGCCATCCTGCACGGCGGCTACCTGG
GQTTGTTTCTCATCATCTTCGCCGCCGTTGTGTGCTGCTACACCGGCAAGATCCTCATCGCGTGCCTGTACGAGGAGAA
TGAAGACGGCGGTGGTGCGCGTGCGGGACTCGTACGCGGCCATAGCCAACGCCTGCTGCGCCCCGCGCTTCCCAACG
CTGGGCGGCCCAGTGGTGAACGTAGCGCAGATGATCGAGCTGGTCATGACGTGCATCCTGTACGTGGTGGTGAGTGGCA
ACCTCATGTACAACAGCTTCCCGGGGCTGCCCGTGTCGCAGAAGTCCTGGTCCATTATCGCCACGGCCGTGCTGCTGCC
TTGCGCCTTCCTTAAGAACCTCAAGGCCGTGTCCAAGTTCACTCTGCTGTGCACTCTGGCCCACTTCGTCATCAATATC
CTGGTCATAGCCTACTGTCTATCGCGGGCGCGCGACTGGGCCTGGGAGAAGGTCAAGTTCTACATCGACGTCAAGAAGT
TCCCCATCTCCATTGGCATCATCGTGTTCAGCTACACGTCTCAGATCTTCCTGCCTTCGCTGGAGGGCAATATGCAGCA
GCCCAGCGAGTTCCACTGCATGATGAACTGGACGCACATCGCAGCCTGCGTGCTCAAGGGCCTCTTCGCGCTCGTCGCC
TACCTCACCTGGGCCGACGACACCAGCAGGCCATCACGGATAACCTCCCCGGCTCCATCCGCGCCGTGGTCAACATCT
TTCTGGTGGCCAAGGCGCTGTTGTCCTATCCTCTGCCATTCTTTGCCGCTGTCGAGGTGCTGGAGAAGTCGCTCTTCCA
GGAAGGCAGCCGCGCCTTTTTCCCGGCCTGCTACAGCGGCGACGGGCGCCTGAAGTCCTGGGGCTGACGCTGCGCTGC
GCGCTCGTCGTCTTCACGCTGCTCATGGCCATTTATGTGCCGCACTTCGCGCTGCTCATGGGCCTCACCGGCAGCCTCA
CGGGCGCCGGCCTCTGTTTCTTGCTGCCCAGCCTCTTTCACCTGCGCCTGCTCTGGCGCAAGCTGCTGTGGCACCAAGT
CTTCTTCGACGTCGCCATCTTCGTCATCGGCGGCATCTGCAGCGTGTCCGGCTTCGTGCACTCCCTCGAGGGCCTCATC
GAAGCCTACCGAACCAACGCGGAGGACTAGGGCGCAAGGGCGAGCCCCCGCCGCGCTTCTGCGCTCTCTCCCTTC

A disclosed NOV7d polypeptide (SEQ ID NO:36) encoded by SEQ ID NO:35 is presented using the one-letter amino acid code in Table 7H. NOV7d amino acid changes, if any, are underlined in Table 7H.

TABLE 7H
Encoded NOV7d protein seqnence.

(SEQ ID NO:36)

MATLLRSKLSNVATSVSNKSQAKMSGMFARMGFQAATDEEAVGFAHCDDLDFEHRQGLQMDILKAEGEPCGDEGAEAPVEGDIHY
QRGSGAPLPPSGSKDQVCGGGEFGGHDKPKITAWEAGWNVTNAIQGMFVLGLPYATLHGGYLGLFLIIFAAVVCCYTGKILIACL
YEENEDGEWRVRDSYAAIANACCAPRFPTLGGRVVNVAQIIELVMTCTLYVVVSCNLMYNSFPGLPVSQKSWSIIATAVLLPCA
PLKNLKAVSKFSLLCTLAEFVINILVIAYCLSRARDWAWEKVKFYIDVKKFPISIGIIVFSYTSQIFLPSLEGNMQQPSEFHGMM
NWTHIAACVLKGLFALVAYLTWADETKEAITDNLPGSIRAVVNIFLVAKALLSYPLPFFAAVEVLEKSLFQEGSRAFFPACYSGD
GRLKSWGLTLRCALWTLLMAIYVPHFALLMGLTGSLTCAGLCFLLPSLFELRLLWRKLLWHQVFFDVAIFVIGGICSVSGFVH
SLEGLIEAYRTNAED

A disclosed NOV7e nucleic acid (also referred to as 13374578) is a variant of NOV7a, encodes a novel vesicular GABA transporter-like protein, and is shown in Table 7I. NOV7e nucleotide changes are underlined in Table 7I.

TABLE 7I
NOV7e Nucleotide Sequence

(SEQ ID NO:37)

GCGGAGAGCAAGCGGAGATAGCGACTTTGCGCCCCCCAGCCCTCGCCTTCTTGCATCGCGTTCCCCGCATCCTCGGGTC
CTTCTGTCCTTTCCGCTGTCCCCACCGCCGCCATGGCCACOTGCTCCGCAGCAAGCTGTCCAACGTGGCCACGTCCGT
GTCCAACAAGPCCCAGGCGAGATGAGCGGCATGTTCGCCAGGATGGGTTTTCAGGCGGCCACGATGAGGAGGCGGTG
GGCTTCGCGCATTGCGACCACCTCGACTTTGAGCACCGCCACGGCCTGCACATGGACATCCTGAAAGCCGAGGGAGAGC
CCTGCGGGACCAGGGCGCTGAAGCGCCCGTCGAGGGACACATCCATTATCAGCGAAGGCAGCGGAGCTCCTCTGCCGCC
CTCCGGCTCCAAGGACCACGTGGGAGGTGGTGGCGAATTCCGGGGCCACGACAAGCCCAAAATTACGGCGTGGGAGGCA
GCCTGGAACGTGACCACGCCATCCAGGGCATGTTCGTGCTGGGCCTACCCTACGCCATCCTGCACGGCGGCTACCTGG
GGTTGTTTCTCATCATCTTCGCCGCCGTTCTGTGCTGCTACACCGGCAAGATCCTCATCGCGTGCCTGTACGAGGAGAA
TGAAGACGGCGAGGTGGTGCGCGTCCGGGACTCGTACGTGGCCATAGCCAACGCCTGCTGCGCCCCGCGCTTCCCAACG
CTGGGCGGCCGAGTGGTGACGTAGCGCAGATCATCCAGCTGGTGATGACGTGCATCCTGTACGTGGTGGTGAGTGGCA
ACCTCATGTACAACAGCTTCCCGGGCTGCCCGTGTCGCAGAAGTCCTGGTCCATTATCGCCAGGCCGTGCTGCTGCC
TTGCGCCTTCCTTAAGAACCTCAAGGCCGTGTCCAAGTTCAGTCTGCTGTGCACTCTGGCCCACTTCGTCATCAATATC
CTGGTCATAGCCTACTGTCTATCGCGGGCGCGCGACTGGGCCTGGGAGAAGGTCAAGTTCTACATCGACGTCAAGAAGT
TCCCCATCTCCATTGGCATCATCGTGTTCAGCTACACGTCTCAGATCTTCCTGCCTTCGCTGGAGGGCAATATGCAGCA
GCCCAGCGAGTTCCACTGCATGATGAACTGGACGCACATCGCAGCCTGCGTGCTCAAGGGCCTCTTCGCTCGTCGCC
TACCTCACCTGGGCCGACGAGACCAAGGAGGCCATCACGGATAACCTGCCCGGCTCCATCCGCGCCGTGGTCAACATCT
TTCTGGTGGCCAAGGCGCTGTTGTCCTATCCTCTGCCATTCTTTGCCGCTGTCGAGGTGCTGGAGAAGTCGCTCTTCCA
GGAAGGCAGCCGCGCCTTTTTCCCGGCCTGCTACAGCGGCGACGGGCGCCTGAAGTCCTGGGGGCTGACGCTGCGCTGC
GCGCTCGTCGTCTTCACGCTGCTCATGGCCATTTATGTGCCGCACTTCGCGCTGCTCATGGGCCTCACCGCAGCCTCA
CGGGCGCCGGCCTCTGTTTCTTGCTGCCCAGCCTCTTTCACCTGCGCCTGCTCTGGCGCAAGCTGCTGTGGCACCAAGT
CTTCTTCGACGTCGCCATCTTCGTCATCGGCGGCATCTGCAGCGTGTCCGGCTTCGTGCACTCCCTCGAGGGCCTCATC
GAAGCCTACCGAACCAACGCGGAGGACTAGGGCGCAAGGGCGAGCCCCCCGCCGCGCTTCTGCGCTCTCTCCCTTC

A disclosed NOV7e polypeptide (SEQ ID NO:38) encoded by SEQ ID NO:37 is presented using the one-letter amino acid code in Table 7J. NOV7e amino acid changes, if any, are underlined in Table 7J.

TABLE 7J
Encoded NOV7e protein sequence.

(SEQ ID NO:38)

MATLLRSKLSNVATSVSNKSQAKMSGMFARMGFQAATDEEAVGFAHCDDLDFEHRQGLQMDILKAEGEPCGDEGAEAPVEGDIHY
QRGSGAPLPPSGSKDQVGGGGEFGGHDKPKITAWEAGWNVTNAIQGMFVLGLPYAILHGGYLGLFLIIFAAVVCCYTGKILIACL
YEENEDGEVVRVRDSYVAIANACCAPRFPTLGGRVVNVAQIIELVMTCILYVVVSGNLMYNSFPGLPVSQKSWSIIATAVLLPCA
FLKNLKAVSKFSLLCTLAHFVINILVIAYCLSRARDWAWEKVKFYIDVKKFPISIGIIVFSYTSQIFLPSLEGNMQQPSEFHCMM
NWTHIAACVLKGLFALVAYLTWADETKEAITDNLPGSIRAVVNIFLVAKALLSYPLPFFAAVEVLESKSLFQEGSRAFFPACYSGD
GRLKSWGLTLRCALVVFTLLMAIYVPHFALLMGLTGSLTGAGLCFLLPSLFHLRLLWRKLLWHQVFFDVAIFVIGGICSVSGFVH
SLEGLIEAYRTNAED

A disclosed NOV7f nucleic acid (also referred to as 13374579) is a variant of NOV7a, encodes a novel vesicular GABA transporter-like protein, and is shown in Table 7K. NOV7f nucleotide changes are underlined in Table 7K.

TABLE 7K
NOV7f Nucleotide Sequence

(SEQ ID NO:39)

GCGGAGAGCAAGCGGAGATAGCGACTTTGCGCCCCCCAGCCCTCGCCTTCTTGCATCGCGTTCCCCGCATCCTCGGGTC
CTTCTGTCCTTTCCGCTGTCCCCACCGCCGCCATGGCCACCTTGCTCCGCAGCAAGCTGCCAACGTGGCCACGTCCGT
GTCCAACAAGTCCCAGGCCAAGATGAGCGGCATGTTCGCCAGGATGGGTTTTCAGGCGGCCACGGATGAGGAGGCGGTG
GGCTTCGCGCATTGCGACGACCTCGACGTTTGAGCACCCCCAGGGCCTGCAGATGCACATCCTGAAAGCCGAGGGAGAGC
CCTGCGGCGACGAGGGCGCTGAAGCGCCCGTCGAGGGACACATCATCAGCGAGGCACGGAGCTCCTCTGCCGCC
CTCCGGCTCCAAGGACCAGGTGGGAGGTGGTGGCGAATTCGGGGACCACGACAAGCCCAAAATCACGGCGTGGGAGGCA
GCCTGGAACGTCACCAACGCCATCCAGGGCATGTTCGTGCTCGGCCTACCCTACGCCATCCTGCGGCGGCTACCTGG
GGTTGTTTCTCATCATCTTCGCCGCCGTTGTGTGCTCCTACACCGGCAAGATCCTCATCGCGTGCCTCTACGAGGAGAA
TGAAGACGGCGACGTGGTGCGCGTGCGCGACTCGTACGTGGCCATAGCCAACGCCTCCTGCGCCCCGCGCTTCCCAACG
CTGGGCGGCCGAGTGGTGAACGTAGCGCAGATCATCGAGCTGGTGATGACGTGGTCCTGTACGTGGTGGTGAGTGGCA
ACCTCATGTACAACAGCTTCCCGGGGCTGCCCGTGTCGCAGAAGTCCTGGTCCATTATCGCCACGGCCGTGCTGCTGCC
TTGCGCCTTCCTTAAGAACCTCAAGGCCGTGTCCAAGTTCAGTCTGCTGTGCACTCTGGCCCACTTCGTCATCAATATC
CTGGTCATAGCCTACTGTCTATCGCGGGCGCGCGACTGGGCCTGGGAGAAGGTCAAGTTCTACATCGACGTCAAGAAGT
TCCCCATCPCCATTGGATCATCGTGTTCAGCTAACGTCTCAGATCTTCCTGCCTTCGCTGGAGGGATATGCAGCA
GCCCAGCGAGTTCCACTGCATGATGACTGCACGCACATCGCAGCCTGCGTGCTCAAGGGCCTCTTCGCGCTCGTCGCC
TACCTCACCTGGGCCGACGAGACCAAGGAAGGCCATCACGGATAACCTGCCCGGCTCCATCCGCGCCGTGGTCAACATCT
TTCTGGTGGCCAAGGCGCTGTTGTCCTATCCTCTGCCATTCTTTGCCGCTGTCGAGGTGCTGGAGAAGTCGCTCTTCCA
GGAAGGCAGCCGCGCCTTTTTCCCGGCCTGCTACAGCGGCGACGGGCGCCTGAAGTCCTGGGGGCTGACGCTGCGCTGC
GCGCTCGTCGTCTTCACGCTGCTCATGGCCATTTATGTGCCGCACTTCGCGCTGCTCATGGGCCTCACCGGCAGCCTCA
CGGGCGCCGGCCTCTGTTTCTTGCTGCCCAGCCTCTTTCACCTGCGCCTGCTCTGGCGCAAGCTGCTGTGGCACCAAGT
CTTCTTCGACGTCGCCATCTTCGTCATCGGCGGCATCTGCAGCGTGTCCGGCTTCGTGCACTCCCTCGAGGGCCTCATC
GAAGCCTACCGAACCAACGCGGAGGACTAGGGCGCAAGGGCCAGCCCCCGCCGCGCTTCTGCGCTCTCTCCCTTC

A disclosed NOV7f polypeptide (SEQ ID NO:40) encoded by SEQ ID NO:39 is presented using the one-letter amino acid code in Table 7L. NOV7f amino acid changes, if any, are underlined in Table 7L.

TABLE 7L
Encoded NOV7f protein sequence.

(SEQ ID NO:40)

MATLLRSKLSNVATSVSNKSQAMSGMFAIGFQAATDEEAVGFAHCDDLDFEHRQGLQMDILKAEGEPCGDEGAEAPVEGDIHY
QRGSGAPLPPSGSKDQVGGGGEFGDHDKPKITAWEAGWNVTNAIQGMFVLGLPYAILHGGYLGLFLIIFAAVVCCYTGKILIACL
YEENEDGEVVRVRDSYVAIANACCAPRFPTLGGRVVNVAQIIELVMTCILYVVVSGNLMYNSFPGLPVSQKSWSIIATAVLLPCA
FLKNLRAVSKFSLLCTLAEFVINILVIAYCLSRARDWAWEKVKFYIDVKKFPISIGIVFSYTSQIFLPSLEGNMQQPSEFHCMM
NWTHIAACVLKGLFALVAYLTWADETKEAITDNLPGSIRAVVNIFLVAKALLSYPLPFFAAVEVLEKSLFQEGSRAFFPACYSGD
GRLKSWGLTLRCALWTLLMAIYVPHFALLMGLTGSLTGAGLCFLLPSLFELRLLWRKLLWHQVFFDVAIFVIGGICSVSGFVH
SLEGLIEAYRTNAED

NOV7a-NOV7f are very closely homologous as is shown in the amino acid alignment in Table 7M.

[image]

[image]

[image]

Homologies to any of the above NOV7 proteins will be shared by the other NOV7 proteins insofar as they are homologous to each other as shown above. Any reference to NOV7 is assumed to refer to all three of the NOV7 proteins in general, unless otherwise noted.

NOV7a also has homology to the amino acid sequence shown in the BLASTP data listed in Table 7N.

TABLE 7N
BLAST results for NOV7a

| Gene Index/ | | Length | Identity | Positives | |
|---|---|---|---|---|---|
| Identifier | Protein/Organism | (aa) | (%) | (%) | Expect |
| gi\|14388326\|dbj\| | hypothetical | 525 | 520/525 | 521/525 | 0.0 |
| BAB60726.1\| | protein | | (99%) | (99%) | |
| (AB062931) | [Macaca fascicularis] | | | | |
| gi\|13929106\|ref\|NP_{—} | vesicular | 525 | 518/526 | 521/526 | 0.0 |
| 113970.1\| | inhibitory amino | | (98%) | (98%) | |
| | acid transporter | | | | |
| | [Rattus | | | | |
| | norvegicus] | | | | |
| gi\|13396317\|emb\| | bA12201.1 (A | 525 | 524/525 | 524/525 | 0.0 |
| CAC15529.2\| | novel protein | | (99%) | (99%) | |
| (AL133519) | (ortholog of the | | | | |
| | mousevesicular | | | | |
| | inhibitory amino | | | | |
| | acid transporter, | | | | |
| | VIAAT) | | | | |
| | [Homo sapiens] | | | | |
| gi\|6678569\|ref\|NP_{—} | vesicular | 521 | 507/522 | 511/522 | 0.0 |
| 033534.1 | inhibitory amino | | (97%) | (97%) | |
| | acid transporter | | | | |
| | [Mus musculus] | | | | |
| gi\|7303217\|gb\| | CG8394 gene | 543 | 203/419 | 282/419 | 1e−110 |
| AAF58280.1\| | product | | (48%) | (66%) | |
| (AE003815) | [Drosophila | | | | |
| | melanogaster] | | | | |

The homology of these sequences is shown graphically in the ClustalW analysis shown in Table 70.

[image]

[image]

[image]

Table 7P lists the domain description from DOMAIN analysis results against NOV7a. This indicates that the NOV7a sequence has properties similar to those of other proteins known to contain this domain.

TABLE 7P
Domain Analysis of NOV7a

gnl Pfam pfam01490, Aa_trans, Transmembrane amino acid transporter
protein. This transmembrane region is found in many amino acid
transporters including UNC-47 and MTR. UNC-47 encodes a vesicular
amino butyric acid (GABA) transporter, (VGAT). UNC-47 is predicted to
have 10 tranamembrane domains . MTR is a N system amino acid
transporter system protein involved in methyltryptophan resistance.
Other members of this family include proline transporters and amino
acid permeases. (SEQ ID NO:198)
Length = 370 residues, 87.8% aligned
Score = 182 bits (461), Expect = 5e−47

| | | | |
|---|---|---|---|
| NOV7a | 143 | HGGYLGLFLIIFAAVVCCYTGKILIACLYEENEDGEVVRVRDSYVAIANACCAPRFPTLG | 202 |
| | | \| \|\| \|++ \| + \|\|\| +\| \| + \|\|\|+ + + + \| | |
| Pfam01490 | 5 | LGWIPGLVLLLLAGFITLYTGLLLSECYE-----YVPGKRNDSYLDLGRSAYGGKGLLLT | 59 |
| NOV7a | 203 | GRVVNVAQIIELVMTCILYVVVSGNLMYNSFP------GLPVSQKSWSIIATAVLLPCAF | 256 |
| | | \| \| + \| \| \|++++\|+\|+ + \|\| \|\| \|+++ +\| | |
| Pfam01490 | 60 | SFVG---QYVNLFGVNIGYLILAGDLLPKIISSFCGDNCDHLDGNSWIIIFAAIIITLSF | 116 |
| NOV7a | 257 | LKNLKAVS--KFSLLCTLAHFVI---NILVIAYCLSRARDWAWEKVKFY---IDVKKFPI | 308 |
| | | + \| +\| \| +\|\|+ \| \|+\| \| + + + + | |
| Pfam01490 | 117 | IPNFNLLSISSLSAFSSLAYLSIISFLIIVAVIAGIFVLLGAVYGILWSPSFTKLTGLFL | 176 |
| NOV7a | 309 | SIGIIVFSYTSQIFLPSLEGNMQQPSE--FHCMMNWTHIAACVLKGLFALVAYLTWADET | 366 |
| | | +\|\|\|\|\|\|++ \| ++ \|+ \|\| \| ++\| \| \|\| \| \|\|\|+ + | |
| Pfam01490 | 177 | AIGIIVFAFEGHAVLLPIQNTMKSPSAKKFKKVLNVAIIIVTVLYILVGFFGYLTFGNNV | 236 |
| NOV7a | 367 | KEVITDNLP-GSIRAVVNIFLVAKALLSYPLPFFAAVEVLEKSLFQEGSRAFFPACYSGD | 425 |
| | | \| \| \|\|\| +\|\|+ \|\| \|\|++\|\| \| \| ++\| \| ++ + \| | |
| Pfam01490 | 237 | KGNILLNLPNNPFWLIVNLNLVVAILLTFPLQAFPIVRIIENLLTKKNNFA--------P | 288 |
| NOV7a | 426 | GRLKSWGLTLRCALVVFTLLMAIYVPHFALLMGLTGSLTGA 466 | |
| | | + \| + +\| \|\|\|\|\|\|\|+\|\| \|\| \| + \| \|+ +\|\| | |
| Pfam01490 | 289 | NKSKLLRVVIRSGLVVFTLLIAILVPFFGDFLSLVGATSGA 329 | |

Synaptic vesicles from mammalian brain are among the best characterized trafficking organelles. However, so far it has not been possible to characterize vesicle subpopulations that are specific for a given neurotransmitter. Taking advantage of the recent molecular characterization of vesicular neurotransmitter transporters, we have used an antibody specific for the vesicular GABA transporter (VGAT) to isolate GABA-specific synaptic vesicles. The isolated vesicles are of exceptional purity as judged by electron microscopy.

Immunoblotting revealed that isolated vesicles contain most of the major synaptic vesicle proteins in addition to VGAT and are devoid of vesicular monoamine and acetylcholine transporters. The vesicles are 10-fold enriched in GABA uptake activity when compared with the starting vesicle fraction. Furthermore, glutamate uptake activity and glutamate-induced but not chloride-induced acidification are selectively lost during immunoisolation. We conclude that the population of GABA-containing synaptic vesicles is separable and distinct from vesicle populations transporting other neurotransmitters. Sagne et al., FEBS Lett 1997:10, 417(2):177-83.

Proteins belonging to the GABA transporter family of proteins play an important role in signal transduction of different cell type such as neuronal and muscle cells. NOV7 protein is the human ortholog of VGAT (vesicular GABA transporter) from Rattus norvegicus and unc-47 from C. elegans which are involved in packaging GABA in synaptic vesicles. NOV7 protein has a domain similar to the amino acid permease domain found in integral membrane proteins that regulate transport of amino acids.

The above defined information for NOV7 suggests that this NOV7 protein may function as a member of a GABA transporter family. Therefore, the NOV7 nucleic acids and proteins of the invention are useful in potential therapeutic applications implicated in various diseases and disorders described below and/or other pathologies. For example, the NOV7 compositions of the present invention will have efficacy for treatment of patients suffering from cancer, trauma, regeneration (in vitro and in vivo), viral/bacterial/parasitic infections, fertility and neurological disorders. The NOV7 nucleic acid encoding GABA transporter receptor-like protein, and the GABA transporter receptor-like protein of the invention, or fragments thereof, may further be useful in diagnostic applications, wherein the presence or amount of the nucleic acid or the protein are to be assessed.

NOV8

NOV8 includes two novel integrin alpha 7 (ITGA7) precursor-like receptor proteins disclosed below. The disclosed proteins have been named NOV8a and NOV8b.

NOV8a

A disclosed NOV8a nucleic acid of 3432 nucleotides (also referred to AC073487_dal) encoding a novel ITGA7 precursor-like receptor protein is shown in Table 8A. An open reading frame was identified beginning with an ATG initiation codon at nucleotides 1-3 and ending with a TAA codon at nucleotides 3430-3432. The start and stop codons are in bold letters.

TABLE 8A
NOV8a Nucleotide Sequence

(SEQ ID NO:41)

ATGGCCGGGGCTCGGAGCCGCGACCCGTTGGGGGGCCTCCGGGATTTGCTACCTTTTTGGCTCCCTGCTCGTCGAACTGC
TCTTCTCACGGCTGTCGCCTTCAATCTGGACGTGATGGGTGCCTTGCGCAAGGAGGCGAGCCAGGCAGCCTCTTCGGCTT
CTCTGTGGCCCTGCACCCGGCACGTCGCAGCCCCGGACCCCAGCAGCCCACTGCTGGTGGGTGCTCCCCAGGCCCTGGCT
CTTCCTGGGCAGCAGGCGAATCGCACTGGAGGCCTCTTCGCTTGCCCGTTGAGCCTGGAGGAGACTGACTGCTACAGAGT
GGACATCGACCAGGGAGCTGATATGCAAAAGGAAAGCAAGGAGAACCAGTGGTTGGGAGTCAGTGTTCGGAGCCAGGGGC
CTGGGGGCAAGATTGTTACCTGTGCACACCGATATGAGGCAAGGCAGCGAGTGGACCAGATCCTGGAGACGCGGGATATG
ATTGGTCGCTGCTTTGTGCTCAGCCAGGACCTGGCCATCCGGGATGAGTTGGATGGTGGGGAATGGAAGTTCTGTGAGGG
ACGCCCCCAAGGCCATGAACAATTTGGGTTCTGCCAGCAGGGCACAGCTGCCGCCTTCTCCCCTGATAGCCACTACCTCC
TCTTTGGGGCCCCAGGAACCTATAATTGGAAGGGCACGGCCAGGGTGGAGCTCTGTGCACAGGGCTCAGCGGACCTGGCA
CACCTGGACGACGGTCCCTACGAGGCGGGGGGAGAGAAGGAGCAGGACCCCCGCCTCATCCCGGTCCCTGCCAACAGCTA
CTTTGGGTTGCTTTTTGTGACCAACATTGATAGCTCAGACCCCGACCAGCTGGTGTATAAAACTTTGGACCCTGCTGACC
GGCTCCCAGGACCAGCCGGAGACTTGGCCCTCAATAGCTACTTAGGCTTCTCTATTGACTCGGGGAAAGGTCTGGTGCGT
CAGTCGCCTGGTGCCCGAGGTTATGCTGTCTGGGGAGCGCCTGACCTCCGGCTTTGGCTACTCACTGGCTGTGGCTGACC
TCAACAGTGATGGGTGGCCAGACCTGATAGTGGGTGCCCCCTACTTCTTTGAGCGCCAAGAAGAGCTGGGGGGTGCTGTG
TATGTGTACTTGAACCAGGGGGGTCACTGGGCTGGGATCTCCCCTCTCCGGCTCTGCGGCTCCCCTGACTCCATGTTCGG
GATCAGCCTGGCTGTCCTGGGGGACCTCAACCAAGATGGCTTTCCAGATATTGCAGTGGGTGCCCCCTTTGATGGTGATG
GGAAAGTCTTCATCTACCATGGGAGCAGCCTGGGGGTTGTCGCCAAACCTTCACAGGTGCTGGAGGGCGAGGCTGTGGGC
ATCAAGAGCTTCGGCTACTCCCTGTCAGGCAGCTTGGATATGGATGGGAACCAATACCCTGACCTGCTGGTGGGCTCCCT
GGCTGACACCGCAGTGCTCTTCAGGGCCAGACCCATCCTCCATGTCTCCCATGAGGTCTCTATTGCTCCACGAAGCATCG
ACCTGGAGCAGCCCAACTGTGCTGGCGGCCACTCGGTCTGTGTGGACCTAAGGGTCTGTTTCAGCTACATTGCAGTCCCC
AGCAGCTATAGCCCTACTGTGGCCCTGGACTATGTGTTAGATGCGGACACAGACCGGAGGCTCCGGGGCCAGGTTCCCCG
TGTGACGTTCCTGAGCCGTAACCTGGAAGAACCCAAGCACCAGGCCTCGGGCACCGTGTGGCTGAAGCACCAGCATGACC
GAGTCTGTGGAGACGCCATGTTCCAGCTCCAGGAAAATGTCAAAGACAAGCTTCGGGCCATTGTAGTGACCTTGTCCTAC
AGTCTCCAGACCCCTCGGCTCCGGCGACAGGCTCCTGGCCAGGGGCTGCCTCCAGTGGCCCCCATCCTCAATGCCCACCA
GCCCAGCACCCAGCGGGCAGAGATCCACTTCCTGAAGCAAGGCTGTGGTGAAGACAAGATCTGCCAGAGCAATCTGCAGC
TGGTCCGCGCCCGCTTCTGTACCCGGGTCAGCGACACGGAATTCCAACCTCTGCCCATGGATGTGGATGGAACAACAGCC
CTGTTTGCACTGAGTGGGCAGCCAGTCATTGGCCTGGAGCTGATGGTCACCAACCTGCCATCGGACCCAGCCCAGCCCCA
GGCTGATGGGGATGATGCCCATGAAGCCCAGCTCCTGGTCATGCTTCCTGACTCACTGCACTACTCAGGGGTCCGGGCCC
TGGACGAGAAGCCACTCTGCCTGTCCAATGAGAATGCCTCCCATGTTGAGTGTGAGCTGGGGAACCCCATGAAGAGAGGT
GCCCAGGTCACCTTCTACCTCATCCTTAGCACCTCCGGGATCAGCATTGAGACCACGGAACTGGAGGTAGAGCTGCTGTT
GGCCACGATCAGTGAGCAGGAGCTGCATCCAGTCTCTGCACGAGCCCGTGTCTTCATTGAGCTGCCACTGTCCATTGCAG
GGATGGCCATTCCCCAGCAACTCTTCTTCTCTGGTGTGGTGAGGGGCGAGAGAGCCATGCAGTCTGAGCGGGATGTGGGC
AGCAAGGTCAAGTATGAGGTCACGGTAAGTAACCAAGGCCAGTCGCTCAGAACCCTGGGCTCTGCCTTCCTCAACATCAT
GTGGCCTCATGAGATTGCCAATGGGAAGTGGTTGCTGTACCCAATGCAGGTTGAGCTGGAGGGCGGGCAGGGGCCTGGGC
AGAAAGGGCTTTGCTCTCCCAGGAGGCCCAACATCCTCCACCTGGATGTGGACAGTAGGGATAGGAGGCGGCGGGAGCTG
GAGCCACCTGAGCAGCAGGAGCCTGGTGAGCGGCAGGAGCCCAGCATGTCCTGGTGGCCAGTGTCCTCTGCTGAGAAGAA
GAAAAACATCACCCTGGACTGCGCCCGGGGCACGGCCAACTGTGTGGTGTTCAGCTGCCCACTCTACAGCTTTGACCGCG
CGGCTGTGCTGCATGTCTGGGGCCGTCTCTGGAACAGCACCTTTCTGGAGGAGTACTCAGCTGTGAAGTCCCTGGAAGTG
ATTGTCCGGGCCAACATCACAGTGAAGTCCTCCATAAAGAACTTGATGCTCCGAGATGCCTCCACAGTGATCCCAGTGAT
GGTATACTTGGACCCCATGGCTGTGGTGGCAGAAGGAGTGCCCTGGTGGGTCATCCTCCTGGCTGTACTGGCTGGGCTGC
TGGTGCTAGCACTGCTGGTGCTGCTCCTGTGGAAGTGTGGCTTCTTCCATCGGAGCAGCCAGAGCTCATCTTTTCCCACC
AACTATCACCGGGCCTGTCTGGCTGTGCAGCCATGGAAGTTGGGGGTCCAGGGACTGTGGGGTAA

The disclosed NOV8a nucleic acid sequence, localized to chromosome 12, has 2531 of 2561 bases (98%) identical to a 3485 bp Homo sapiens integrin alpha-7 mRNA (GENBANK-ID: AF072132|acc:AF072132) (E=0.0).

A disclosed NOV8a polypeptide (SEQ ID NO:42) encoded by SEQ ID NO:41 is 1143 amino acid residues and is presented using the one-letter amino acid code in Table 8B. Signal P, Psort and/or Hydropathy results predict that NOV8a does not contain a signal peptide and is likely to be localized to the endoplasmic reticulum or nucleus with a certainty of 0.6000.

TABLE 8B
Encoded NOV8a protein seqnence.

(SEQ ID NO:42)

MAGARSRDPLGGLRDLLPFWLPARRTALLTAVAFNLDVMGALRKEASQAASSASLWPCTRHVAAPDPSSPL
LVGAPQALALPGQQANRTGGLFACPLSLEETDCYRVDIDQGADMQKESKENQWLGVSVRSQGPGGKIVTCA
HRYEARQRVDQILETRDMIGRCFVLSQDLAIRDELDGGEWKFCEGRPQGHEQFGFCQQGTAAAFSPDSHYL
LFGAPGTYNWKGTARVELCAQGSADLAHLDDGPYEAGGEKEQDPRLIPVPANSYFGLLFVTNIDSSDPDQL
VYKTLDPADRLPGPAGDLALNSYLGFSIDSGKGLVRAEELSFVAGAPRANHKGAVVILRKDSASRLVPEVM
LSGERLTSGFGYSLAVADLNSDGWPDLIVGAPYFFERQEELGGAVYVYLNQGGHWAGISPLRLCGSPDSMF
GISLAVLGDLNQDGFPDIAVGAPFDGDGKVFIYHGSSLGVVAKPSQVLEGEAVGIKSFGYSLSGSLDMDGN
QYPDLLVGSLADTAVLFRARPILHVSHEVSIAPRSIDLEQPNCAGGHSVCVDLRVCFSYIAVPSSYSPTVA
LDYVLDADTDRRLRGQVPRVTFLSRNLEEPKHQASGRVWLKHQHDRVCGDAMFQLQENVKDKLRAIVVTLS
YSLQTPRLRRQAPGQGLPPVAPILNAHQPSTQRAEIHFLKQGCGEDKICQSNLQLVRARFCTRVSDTEFQP
LPMDVDGTTALFALSGQPVIGLELMVTNLPSDPAQPQADGDDAHEAQLLVMLPDSLHYSGVRALDEKPLCL
SNENASHVECELGNPMKRGAQVTFYLILSTSGISIETTELEVELLLATISEQELHPVSARARVFIELPLSI
AGMAIPQQLFFSGVVRGERAMQSERDVGSKVKYEVTVSNQGQSLRTLGSAFLNIMWPHEIANGKWLLYPMQ
VELEGGQGPGQKGLCSPRRPNILHLDVDSRDRRRRELEPPEQQEPGERQEPSMSWWPVSSAEKKKNITLDC
ARGTANCVVFSCPLYSFDRAAVLHVWGRLWNSTFLEEYSAVKSLEVIVRANITVKSSIKNLMLRDASTVIP
VMVYLDPMAVVAEGVPWWVILLAVLAGLLVLALLVLLLWKCGFFHRSSQSSSFPTNYHRACLAVQPSAMEV
GGPGTVG

The NOV8a amino acid sequence has 975 of 1113 amino acid residues (87%) identical to, and 1032 of 1113 amino acid residues (92%) similar to, the Mus musculus 1161 amino acid residue integrin alpha 7 precursor protein (SPTREMBL-ACC: 088731)(E=0.0).

NOV8b

A disclosed NOV8b nucleic acid of 3110 nucleotides (also referred to CG53926-02) encoding a novel ITGA7 precursor-like receptor protein is shown in Table 8C. An open reading frame was identified beginning with an ATG initiation codon at nucleotides 1-3 and ending with a TAA codon at nucleotides 3106-3108. A putative untranslated region downstream from the termination codon is underlined in Table 8C, and the start and stop codons are in bold letters.

TABLE 8C
NOV8b Nucleotide Sequence

(SEQ ID NO:43)

ATGGCCGGGGCTCGGAGCCGCGACCCGTTGGGGGGCCTCCGGGATTTGCTACCTTTTTGGCTCCCTGCTCG
TCGAACTGCTCTTCTCACGGCTGTCGCCTTCAATCTGGACGTGATGGGTGCCTTGCGCAAGGAGGAGAGCC
AGGCAGCCTCTTCGGCTTCTCTGTGGCCCTGCACCCGGCACGTCGCAGCCCCGGACCCCAGCAGCCCACTG
CTGGTGGGTGCTCCCCAGGCCCTGGCTCTTCCTGGGCAGCAGGCGAATCGCACTGGAGGCCTCTTCGCTTG
CCCGTTGAGCCTGGAGGAGACTGACTGCTACAGAGTGGACATCGACCAGGGAGCTGATATGCAAAAGGAAA
GCAAGGAGAACCAGTGGTTGGGAGTCAGTGTTCGGAGCCAGGGGCATTGGTCGCTGCTTTGTGCTCAGCCA
GGACCTGGCCATCCGGGATGAGTTGGATGGTGGGGAATGGAAGTTCTGTGAGGGACGCCCCCAAGGCCATG
AACAATTTGGGTTCTGCCAGCAGGGCACAGCTGCCGCCTTCTCCCCTGATAGCCACTACCTCCTCTTTGGG
GCCCCAGGAACCTATAATTGGAAGGGCACGGCCAGGGTGGAGCTCTGTGCACAGGGCTCAGCGGACCTGGC
ACACCTGGACGACGGTCCCTACGAGGCGGGGGGAGAGAAGGAGCAGGACCCCCGCCTCATCCCGGTCCCTG
CCAACAGCTACTTTGGGTTGCTTTTTGTGACCAACATTGATAGCTCAGACCCCGACCAGCTGGTGTATAAA
ACTTTGGACCCTGCTGACCGGCTCCCAGGACCAGCCGGAGACTTGGCCCTCAATAGCTACTTAGGCTTCTC
TATTGACTCGGGGAAAGGTCTGGTGCGTGCAGAAGAGCTGAGCTTTGTGGCTGGAGCCCCCCGCGCCAACC
ACAAGGGTGCTGTGGTCATCCTGCGCAAGGACAGCGCCAGTCGCCTGGTGCCCGAGGTTATGCTGTCTGGG
GAGCGCCTGACCTCCGGCTTTGGCTACTCACTGGCTGTGGCTGACCTCAACAGTGATGGGTGGCCAGACCT
GATAGTGGGTGCCCCCTACTTCTTTGAGCGCCAAGAAGAGCTGGGGGGTGCTGTGTATGTGTACTTGAACC
AGGGGGGTCACTGGGCTGGGATCTCCCCTCTCCGGCTCTGCGGCTCCCCTGACTCCATGGTAGGGATCAGC
CTGGCTGTCCTGGGGGACCTCAACCAAGATGGCTTTCCAGATATTGCAGTGGGTGCCCCCTTTGATGGTGA
TGGGAAAGTCTTCATCTACCATGGGAGCAGCCTGGGGGTTGTCGCCAAACCTTCACAGGTGCTGGAGGGCG
AGGCTGTGGGCATCAAGAGCTTCGGCTACTCCCTGTCAGGCAGCTTGGATATGGATGGGAACCAATACCCT
GACCTGCTGGTGGGCTCCCTGGCTGACACCGCAGTGCTCTTCAGGGCCAGACCCATCCTCCATGTCTCCCA
TGAGGTCTCTATTGCTCCACGAAGCATCGACCTGGAGCAGCCCAACTGTGCTGGCGGCCACTCGGTCTGTG
TGGACCTAAGGGTCTGTTTCAGCTACATTGCAGTCCCCAGCAGCTATAGCCCTACTGTGGCCCTGGACTAT
GTGTTAGATGCGGACACAGACCGGAGGCTCCGGGGCCAGGTTCCCCGTGTGACGTTCCTGAGCCGTAACCT
GGAAGAACCCAAGCACCAGGCCTCGGGCACCGTGTGGCTGAAGCACCAGCATGACCGAGTCTGTGGAGACG
CCATGTTCCAGCTCCAGGAAAATGTCAAAGACAAGCTTCGGGCCATTGTAGTGACCTTGTCCTACAGTCTC
CAGACCCCTCGGCTCCGGCGACAGGCTCCTGGCCAGGGGCTGCCTCCAGTGGCCCCCATCCTCAATGCCCA
CCAGCCCAGCACCCAGCGGGCAGAGATCCACTTCCTGAAGCAAGGCTGTGGTGAAGACAAGATCTGCCAGA
GCAATCTGCAGCTGGTCCGCGCCCGCTTCTGTACCCGGGTCAGCGACACGGAATTCCAACCTCTGCCCATG
GATGTGGATGGAACAACAGCCCTGTTTGCACTGAGTGGGCAGCCAGTCATTGGCCTGGAGCTGATGGTCAC
CAACCTGCCATCGGACCCAGCCCAGCCCCAGGCTGATGGGGATGATGCCCATGAAGCCCAGCTCCTGGTCA
TGCTTCCTGACTCACTGCACTACTCAGGGGTCCGGGCCCTGGACCCTGCGGAGAAGCCACTCTGCCTGTCC
AATGAGAATGCCTCCCATGTTGAGTGTGAGCTGGGGAACCCCATGAAGAGAGGTGCCCAGGTCACCTTCTA
CCTCATCCTTAGCACCTCCGGGATCAGCATTGAGACCACGGAACTGGAGGTAGAGCTGCTGTTGGCCACGA
TCAGTGAGCAGGAGCTGCATCCAGTCTCTGCACGAGCCCGTGTCTTCATTGAGCTGCCACTGTCCATTGCA
GGAATGGCCATTCCCCAGCAACTCTTCTTCTCTGGTGTGGTGAGGGGCGAGAGAGCCATGCAGTCTGAGCG
GGATGTGGGCAGCAAGGACTGCGCCCGGGGCACGGCCAACTGTGTGGTGTTCAGCTGCCCACTCTACAGCT
TTGACCGCGCGGCTGTGCTGCATGTCTGGGGCCGTCTCTGGAACAGCACCTTTCTGGAGGAGTACTCAGCT
GTGAAGTCCCTGGAAGTGATTGTCCGGGCCAACATCACAGTGAAGTCCTCCATAAAGGACTTGATGCTCCG
AGATGCCTCCACAGTGATCCCAGTGATGGTATACTTGGACCCCATGGCTGTGGTGGCAGAAGGAGTGCCCT
GGTGGGTCATCCTCCTGGCTGTACTGGCTGGGCTGCTGGTGCTAGCACTGCTGGTGCTGCTCCTGTGGAAG
TGTGGCTTCTTCCATCGGAGCAGCCAGAGCTCATCTTTTCCCACCAACTATCACCGGGCCTGTCTGGCTGT
GCAGCCTTCAGCCATGGAAGTTGGGGGTCCAGGGACTGTGGGGTAACT

The disclosed NOV8b nucleic acid sequence, localized to chromosome 12, has 1856 of 1867 bases (99%) identical to a Homo sapiens integrin alpha-7 mRNA (gb:GENBANK-ID:AF032108|acc:AF032108.1) (E=0.0).

A disclosed NOV8b polypeptide (SEQ ID NO:44) encoded by SEQ ID NO:43 is 1035 amino acid residues and is presented using the one-letter amino acid code in Table 8D. Signal P, Psort and/or Hydropathy results predict that NOV8b does not contain a signal peptide and is likely to be localized to the endoplasmic reticulum with a certainty of 0.8500.

TABLE 8D
Encoded NOV8b protein seqnence.

(SEQ ID NO:44)

MAGARSRDPLGGLRDLLPFWLPARRTALLTAVAFNLDNVGALRKEASQAASSASLWPCTRHVAAPDPSSPL
LVGAPQALALPGQQANRTGGLFACPLSLEETDCYRVDIDQGADMQKESKENQWLGVSVRSQGPGGKIVTCA
HRYEARQRVKQILETRDMIGRCFVLSQDLAIRDELDGGEWKFCEGRPQGHEQFGFCQQGTAAAFSPKSHYL
LFGAPGTYNWKGTARVELCAQGSADLAHLDDGPYEAGGEKEQDPRLIPVPANSYFGLLFVTNIDSSDPDQL
VYKTLDPADRLPGPAGDLALNSYLGFSIDSGKGLVRAEELSFVAGAPRANHKGAVVILRKDSASRLVPEVM
LSGERLTSGFGYSLAVADLNSDGWPDLIVGAPYFFERQEELGGAVYVYLNQGGHWAGISPLRLCGSPDSMF
GISLAVLGDLNQDGFPDIAVGAPFDGDGKVFIYHGSSLGVVAKPSQVLEGEAVGIKSFGYSLSGSLDMDGN
QYPDLLVGSLADTAVLFRARPILHVSHEVSIAPRSIDLEQPNCAGGHSVCVDLRVCFSYIAVPSSYSPTVA
LDYVLDADTDRRLRGQVPRVTFLSRNLEEPKHQASGTVWLKHQHDRVCGDAMFQLQENVKDKLRAIVVTLS
YSLQTPRLRRQAPGQGLPPVAPILNAHQPSTQRAEIHFLKQGCGEDKICQSNLQLVRARFCTRVSDTEFQP
LPMDVDGTTALFALSGQPVIGLELMVTNLPSDPAQPQADGDDAHEAQLLVMLPDSLHYSGVRALDPAEKPL
CLSNENASHVECELGNPMKRGAQVTFYLILSTSGISIETTELEVELLLATISEQELHPVSARARVFIELPL
SIAGMAIPQQLFFSGVVRGERAMQSERDVGSKDCARGTANCVVFSCPLYSFDRAAVLHVWGRLWNSTFLEE
YSAVKSLEVIVRANITVKSSIKDLMLRDASTVIPVMVYLDPMAVVAEGVPWWVILLAVLAGLLVLALLVLL
KWJCGFFGRSSQSSSFOTBTGRACKAVQOSANEVGGOGTVG

The NOV8b amino acid sequence has 843 of 884 amino acid residues (95%) identical to, and 844 of 884 amino acid residues (95%) similar to, the Homo sapiens 1181 amino acid residue integrin alpha-7 precursor protein (ptnr:SWISSNEW-ACC:Q13683) (E=0.0).

NOV8b is expressed in at least the following tissues: skeletal muscle, cardiac muscle, small intestine, colon, ovary, prostate, lung and testis.

The NOV8a and 8b proteins are very closely homologous as shown in the alignment in Table 8E.

[image]

[image]

[image]

Homologies to either of the above NOV8 proteins will be shared by the other NOV8 protein insofar as they are homologous to each other as shown above. Any reference to NOV8 is assumed to refer to both of the NOV8 proteins in general, unless otherwise noted.

The disclosed NOV8 polypeptide has homology to the amino acid sequences shown in the BLASTP data listed in Table 8F.

TABLE 8F
BLAST results for NOV8a

| Gene Index/ | | Length | Identity | Positives | |
|---|---|---|---|---|---|
| Identifier | Protein/Organism | (aa) | (%) | (%) | Expect |
| gi\|6680480\|ref\|NP_{—} | integrin alpha 7 | 1135 | 899/1095 | 960/1095 | 0.0 |
| 032424.1\| | [Mus musculus] | | (82%) | (87%) | |
| gi\|12643785\|sp\| | INTEGRIN ALPHA-7 | 1179 | 941/1095 | 1002/1095 | 0.0 |
| Q61738\| | PRECURSOR | | (85%) | (90%) | |
| ITA7_MOUSE | [Mus musculus] | | | | |
| gi\|4504753\|ref\|NP_{—} | integrin alpha 7 | 1137 | 1025/1125 | 1029/1125 | 0.0 |
| 002197.1\| | precursor | | (91%) | (91%) | |
| | [Homo sapiens] | | | | |
| gi\|3158408\|gb\| | integrin alpha 7 | 1137 | 1023/1125 | 1027/1125 | 0.0 |
| AAC18968.1\| | [Homo sapiens] | | (90%) | (90%) | |
| (AF052050) | | | | | |
| gi\|7447667\|pir\|: | integrin alpha 7 | 1062 | 617/702 | 619/702 | e−157 |
| JC5951 | chain variant | | (87%) | (87%) | |
| | [Homo sapiens] | | | | |

The homology between these and other sequences is shown graphically in the ClustalW analysis shown in Table 8G.

[image]

[image]

[image]

[image]

[image]

Table 8H-J lists the domain description from DOMAIN analysis results against NOV8a. This indicates that the NOV8a sequence has properties similar to those of other proteins known to contain these domains.

TABLE 811
Domain Analysis of NOV8a

gnl Smart|smart00191, Int_alpha, Integrin alpha (beta-propellor
repeats).; Integrins are cell adhesion molecules that mediate cell-
extracellular matrix and cell-cell interactions. They contain both
alpha and beta subunits. Alpha integrins are proposed to contain a
domain containing a 7-fold repeat that adopts a beta-propellor fold.
Some of these domains contain an inserted von Willebrand factor type-A
domain. Some repeats contain putative calcium-binding sites. The 7-
fold repeat domain is homologous to a similar domain in
phosphatidylinositOlglycansPecific phospholipase D. (SEQ ID NO:113)
Length=56 residues, 100.0% aligned
Score=62.4 bits (150), Expect=1e-10

| | | | |
|---|---|---|---|
| NOV8a | 422 | PDSMFGISLAVLGDLNQDGFPDIAVGAPFDGD---GKVFIYHGSSLGVVAKPSQVLE | 475 |
| | | \| \| \|\| \|+\| +\|\|+\| \|\|+\|\|+ \|\|\|\| \| \| \|++\| \|\|\| \| \| \| \| | |
| Smart00191 | 1 | PGSYFGYSVAGVGDVNGDGYPDLLVGAPRANDAETGAVYVYFGSS-GGRCIPLQNLS | 56 |

TABLE 81
Domain Analysis of NOV8a

gnl Smart|smart00191, Int_alpha. Integrin alpha (beta-propellor
repeats. (SEQ ID NO:114)
Length=56 residues, 96.4% aligned
Score=53.1 bits (126), Expect 8e-08

| | | | |
|---|---|---|---|
| NOV8a | 363 | SGFGYSLA-VADLNSDGWPDLIVGAPYFFERQEELGGAVYVYL-NQGGHWAGISPLR | 417 |
| | | \| \|\|\|\|+\| \| \|+\| \|\|+\|\|\|+\|\|\|\| + + \|\|\|\|\|\| + \|\| + \| | |
| Smart00191 | 3 | SYFGYSVAGVGDVNGDGYPDLLVGAPRANDAE---TGAVYVYFGSSGGRCIPLQNLS | 56 |

TABLE 8J
Domain Analysis of NOV8a

gnl Smart|smart00191, Int_alpha, Integrin alpha (beta-propellor
repeats). (SEQ ID NO:1lS)
Length=56 residues, 98.2% aligned
Score=38.1 bits (87), Expect=0.003

| | | | |
|---|---|---|---|
| NOV8a | 305 | NSYLGFSIDSGKGLVRAEELSFVAGAPRAN--HKGAVVILRKDSASRLVPEVMLS | 357 |
| | | \|\| \|+\|+ + + \|\|\|\|\|\| \|\|\| + \| \| +\| \|\| | |
| Smart00191 | 2 | GSYFGYSVAGVGDVNGDGYPDLLVGAPRANDAETGAVYVYFGSSGGRCIPLQNLS | 56 |

Expression of the alpha-7 integrin gene (ITGA7) is developmentally regulated during the formation of skeletal muscle. Increased levels of expression and production of isoforms containing different cytoplasmic and extracellular domains accompany myogenesis. From examining the rat and human genomes by Southern blot analysis and in situ hybridization, Wang et al. (Genomics 26: 563-570, 1995) determined that both genomes contain a single alpha-7 gene. In the human, ITGA7 is present on 12q13, as localized by fluorescence in situ hybridization (Wang et al., 1995). Phylogenetic analysis of the integrin alpha-chain sequences suggested that the early integrin genes evolved in 2 pathways to form the I-integrins and the non-I-integrins. The I-integrin alpha chains apparently arose as a result of an early insertion into the non-I-gene. The I-chain subfamily further evolved by duplications within the same chromosome. The non-I-integrin alpha-chain genes are located in clusters on chromosomes 2, 12, and 17, which coincides closely with the localization of the human homeobox gene clusters. Non-I-integrin alpha-chain genes appear to have evolved in parallel and in proximity to the HOX clusters. Thus, the HOX genes that underlie the design of body structure and the integrin genes that underlie informed cell-cell and cell-matrix interactions appear to have evolved in parallel and coordinate fashions.

ITGA7 is a specific cellular receptor for the basement membrane protein laminin-1, as well as for the laminin isoforms-2 and -4. The alpha-7 subunit is expressed mainly in skeletal and cardiac muscle and may be involved in differentiation and migration processes during myogenesis. Three cytoplasmic and 2 extracellular splice variants are developmentally regulated and expressed in different sites in the muscle. In adult muscle, the alpha-7A and alpha-7B subunits are concentrated in myotendinous junctions but can also be detected in neuromuscular junctions and along the sarcolemmal membrane. To study the involvement of alpha-7 integrin during myogenesis and its role in muscle integrity and function, Mayer et al. (Nature Genet. 17: 318-323, 1997) generated a null allele of the ITGA7 gene in the germline of mice by homologous recombination in embryonic stem (ES) cells. To their surprise, mice homozygous for the mutation were viable and fertile, indicating that the gene is not essential for myogenesis. However, histologic analysis of skeletal muscle showed typical signs of progressive muscular dystrophy starting soon after birth, but with a distinct variability in different muscle types. The histopathologic changes indicated an impairment of function of the myotendinous junctions. Thus, ITGA7 represents an indispensable linkage between the muscle fiber and extracellular matrix that is independent of the dystrophin-dystroglycan complex-mediated interaction of the cytoskeleton with the muscle basement membrane.

The basal lamina of muscle fibers plays a crucial role in the development and function of skeletal muscle. An important laminin receptor in muscle is integrin alpha-7/beta-1D. Integrin beta-1 (ITGB1; 135630) is expressed throughout the body, while integrin alpha-7 is more muscle-specific. To address the role of integrin alpha-7 in human muscle disease, Hayashi et al. (Nature Genet. 19: 94-97, 1998) determined alpha-7 protein expression in muscle biopsies from 117 patients with unclassified congenital myopathy and congenital muscular dystrophy by immunocytochemistry. They found 3 unrelated patients with integrin alpha-7 deficiency and normal laminin alpha-2 chain expression. (Deficiency of LAMA2 (156225) causes congenital muscular dystrophy, and a secondary deficiency of integrin alpha-7 was observed in some cases.) The 3 patients were found to carry mutations in the ITGA7 gene. Hayashi et al. (1998) noted that the finding in these patients accords well with the findings in Itga7 knockout mice (Mayer et al., 1997).

The protein similarity information, expression pattern, and map location for the NOV 8 (ITGA7-like) protein and nucleic acid disclosed herein suggest that NOV8 may have important structural and/or physiological functions characteristic of the ITGA7 family. Therefore, the NOV8 nucleic acids and proteins of the invention are useful in potential therapeutic applications implicated in various diseases and disorders described below and/or other pathologies. For example, the NOV8 compositions of the present invention will have efficacy for treatment of patients suffering from Eosinophilic myeloproliferative disorder, Pseudohypoaldosteronism, type IIC, Pseudohypoaldosteronism typeI Spastic paraplegia-10, Hemolytic anemia due to triosephosphate isomerase deficiency, Immunodeficiency with hyper-IgM, type 2, Clr/Cls deficiency, combined, Cls deficiency, isolated, Leukemia, acute lymphoblastic, Periodic fever, familial, Hypertension, Episodic ataxia/myokymia syndrome, Immunodeficiency with hyper-IgM, type 2, Muscular dystrophy, Lesch-Nyhan syndrome, Myasthenia gravis and other muscular and cellular adhesion disorders. The NOV8 nucleic acid encoding ITGA7-like protein, and the ITGA7-like protein of the invention, or fragments thereof, may further be useful in diagnostic applications, wherein the presence or amount of the nucleic acid or the protein are to be assessed.

NOV9

NOV9 includes six novel TMS-2-like proteins disclosed below. The disclosed proteins have been named NOV9a, NOV9b, NOV9c, NOV9d, NOV9e and NOV9f.

NOV9a

A disclosed NOV9a nucleic acid of 1374 nucleotides (also referred to 124141642_EXT_dal) encoding a novel TMS-2-like protein is shown in Table 9A. An open reading frame was identified beginning with an ATG initiation codon at nucleotides 1-3 and ending with a TGA codon at nucleotides 1372-1374. The start and stop codons are in bold letters.

TABLE 9A
NOV9a Nucleotide Sequence

(SEQ ID NO:45)

ATGGGGGCCTGCCTGGGAGCCTGCTCCCTGCTCAGCTGCGTGAGTCCTGCTGGCTGTGCGTCCTCCCTCTG
CGGCTCTGCCCCCTGCATCCTGTGCAGCTGCTGCCCCGCCAGCCGCAACTCCACCGTGAGCCGCCTCATCT
TCACGTTCTTCCTCTTCCTGGCGGTGTTGGTGTCCATCATTATGCTGAGCCCGGGCGTGGAGAGTCAGCTC
TACAAGCTGCCCTGGGTGTGTGAGGAGGGGGCCGGGATCCCCACCGTCCTGCAGGGCCACATCGACTGTGG
CTCCCTGCTTGGCTACCGCGCTGTCTACCGCATGTGCTTCGCCACGGCGGCCTTCTTCTTCTTTTTCACCC
TGCTCATGCTCTGCGTGAGCAGCAGCCGGGACCCCCGGGCTGCCATCCAGAATGGGTTTTGGTTCTTTAAG
TTCCTGATCCTGGTCGGCCTCACCGTGGGTGCCTTCTACATTCCTGACGGCTCCTTCACCAACATCTGGTT
CTACTTCGGCGTCGTGGGCTCCTTCCTCTTCATCCTCATCCAGCTGGTGCTGCTCATCGACTTTGCGCACT
CCTGGAACCAGCGGTGGCTGGGCAAGGCCGAGGAGTGCGATTCCCGTGCCTGGTACGCATCACTCTCCTCT
TCTACTTGTCTGTCGATCGCGGCCGTGGCGCTGATGTTCATGTACTACACTGAGCCCAGCGGCTGCCACGA
GGGCAAGGTCTTCATCAGCCTCAACCTCACCTTCTGTGTCTGCGTGTCCATCGCTGCTGTCCTGCCCAAGG
TCCAGGTGAGCCTGCCTAACTCGGGTCTGCTGCAGGCCTCGGTCATCACCCTCTACACCATGTTTGTCACC
TGGTCAGCCCTATCCAGTATCCCTGAACAGAAATGCAACCCCCATTTGCCAACCCAGCTGGGCAACGAGAC
AGTTGTGGCAGGCCCCGAGGGCTATGAGACCCAGTGGTGGGATGCCCCGAGCATTGTGGGCCTCATCATCT
TCCTCCTGTGCACCCTCTTCATCAGTCTGCGCTCCTCAGACCACCGGCAGGTGAACAGCCTGATGCAGACC
GAGGAGTGCCCACCTATGCTAGACGCCACACAGCAGCAGCAGCAGGTGGCAGCCTGTGAGGGCCGGGCCTT
TGACAACGAGCAGGACGGCGTCACCTACAGCTACTCCTTCTTCCACTTCTGCCTGGTGCTGGCCTCACTGC
ACGTCATGATGACGCTCACCAACTGGTACAAGTGCGTAGAGACCCGGAAGATGATCAGCACGTGGACCGCC
GTGTGGGTGAAGATCTGTGCCAGCTGGGCAGGGCTGCTCCTCTACCTGTGGACCCTGGTAGCCCCACTCCT
CCTGCGCAACCGCGACTTCAGCTGA

The disclosed NOV9a nucleic acid sequence, localized to chromosome 1, has 359 of 554 bases (64%) identical to a 1759 bp Homo sapiens transmembrane protein SBBI99 mRNA from (GENBANK-ID: AF153979|acc:AF153979) (E=4.5e⁻⁵⁰).

A disclosed NOV9a polypeptide (SEQ ID NO:46) encoded by SEQ ID NO:45 is 457 amino acid residues and is presented using the one-letter amino acid code in Table 9B. Signal P, Psort and/or Hydropathy results predict that NOV8a has a signal peptide and is likely to be localized to the plasma membrane with a certainty of 0.6760. The most likely cleavage site for a NOV9a peptide is between amino acids 69 and 70, at: VES-QL.

TABLE 9B
Encoded NOV9a protein sequence.

(SEQ ID NO:46)

MGACLGACSLLSCVSPAGCASCLCGSAPCILCSCCPASRNSTVSRLIFTFFLFLGVLVSIIMLSPGVESQL
YKLPWVCEEGAGIPTVLQGHIDCGSLLGYRAVYRMCFATAAFFFFFTLLMLCVSSSRDPRAAIQNGFWFFK
FLILVGLTVGAFYIPDGSFTNIWFYFGVVGSFLFILIQLVLLIDFAHSWNQRWLGKAEECDSRAWYASLSS
STCLSIAAVALMFMYYTEPSGCHEGKVFISLNLTFCVCVSIAAVLPKVQVSLPNSGLLQASVITLYTMFVT
WSALSSIPEQKCNPHLPTQLGNETVVAGPEGYETQWWDAPSIVGLIIFLLCTLFISLRSSDHRQVNSLMQT
EECPPMLDATQQQQQVAACEGRAFDNEQDGVTYSYSFFHCLVLASLHVMMTLTNWYKCVETRKMISTWTA
VWVKICASWAGLLLYLWTLVAPLLLRNRDFS

The NOV9a amino acid sequence has 249 of 456 amino acid residues (54%) identical to, and 328 of 456 amino acid residues (71%) similar to, the Mus musculus 453 amino acid residue membrane protein TMS-2 protein (SPTREMBL-ACC: Q9QZI8) (E=2.1e⁻¹³⁵).

NOV9a also has homology to the amino acid sequences shown in the BLASTP data listed in Table 9C.

TABLE 9C
BLAST results for NOV9a

| Gene Index/ | | Length | Identity | Positives | |
|---|---|---|---|---|---|
| Identifier | Protein/Organism | (aa) | (%) | (%) | Expect |
| gi\|15077634\|gb\| | FKSG84 [Homo | 456 | 437/462 | 438/462 | 0.0 |
| AAK83284.1\| | sapiens] | | (94%) | (94%) | |
| AF352325_1 | | | | | |
| (AF352325) | | | | | |
| gi\|9790269\|ref\|NP_{—} | tumor | 453 | 248/465 | 327/465 | 1e−131 |
| 062734.1 | differentially | | (53%) | (69%) | |
| | expressed 1, like; | | | | |
| | membrane protein TMS-2 | | | | |
| | [Mus musculus] | | | | |
| gi\|11282574\|pir\| | hypothetical | 457 | 249/465 | 328/465 | 1e−126 |
| T46332 | protein | | (53%) | (69%) | |
| | DKFZp434H0413.1 | | | | |
| | [Homo sapiens] | | | | |
| gi\|4750715\|ref\|XP_{—} | KIAA1253 protein | 453 | 249/465 | 328/465 | 1e−125 |
| 051568.1\| | [Homo sapiens] | | (53%) | (69%) | |
| gi\|6382026\|dbj\| | KIAA1253 protein | 472 | 249/465 | 328/465 | 1e−125 |
| BAA86567.1\| | [Homo sapiens] | | (53%) | (69%) | |
| (AB033079) | | | | | |

The homology of these sequences is shown graphically in the ClustalW analysis shown in Table 9D.

[image]

[image]

[image]

Novel variants for the NOV9a nucleic acid and TMS-2-like protein are also disclosed herein as variants of NOV9a. Variants, as described above, are reported individually, but any combination of all or a subset are also included.

A disclosed NOV9b nucleic acid (also referred to as 13375406) is a variant of NOV9a, encodes a novel TMS-2-like protein, and is shown in Table 9E. NOV9b nucleotide changes are underlined in Table 9E.

TABLE 9E
NOV9b Nucleotide Sequence

(SEQ ID NO:47)

ATGGGGGCCTGCCTGGGAGCCTGCTCCCTGCTCAGCTGCGTGAGTCCTGCTGGCTGTGCGTCCTGCCTCTGCGGCTCTG
CCCCCTGCATCCTGTGCAGCTGCTGCCCCGCCAGCCGCAACTCCACCGTGAGCCGCCTCATCTTCACGTTCTTCCTCTT
CCTGGGGGTGTTGGTGTCCATCATTATGCTGAGCCCGGGCGTGGAGAGTCAGCTCTACAAGCTGCCCTGGGTGTGTGAG
GAGGGGGCCGGGATCCCCACCGTCCTGCAGGGCCACATCGACTGTGGCTCCCTGCTTGGCTACCGCGCTGTCTACCGCA
TGTGCTTCGCCACGGCGGCCTTCTTCTTCTTTTTCACCCTGCTCATGCTCTGCGTGAGCAGCAGCCGGGACCCCCGGGC
TGCCATCCAGAATGGGTTTTGGTTCTTTAAGTTCCTGATCCTGGTGGGCCTCACCGTGGGTGCCTTCTACATCCCTGAC
GGCTCCTTCACCAACATCTGGTTCTACTTCGGCGTCGTGGGCTCCTTCCTCTTCATCCTCATCCAGCTGGTGCTGCTCA
TCGACTTTGCGCACTCCTGGAACCAGCGGTGGCTGGGCAAGGCCGAGGAGTGCGATTCCCGTGCCTGGTACGCATCACT
CTCCTCTTCTACTTGTCTGTCGATCGCGGCCGTGGCGCTGATGTTCATGTACTACACTGAGCCCAGCGGCTGCCACGAG
GGCAAGGTCTTCATCAGCCTCAACCTCACCTTCTGTGTCTGCGTGTCCATCGCTGCTGTCCTGCCCAAGGTCCAGGTGA
GCCTGCCTAACTCGGGTCTGCTGCAGGCCTCGGTCATCACCCTCTACACCATGTTTGTCACCTGGTCAGCCCTATCCAG
TATCCCTGAACAGAAATGCAACCCCCATTTGCCAACCCAGCTGGGCAACGAGACAGTTGTGGCAGGCCCCGAGGGCTAT
GAGACCCAGTGGTGGGATGCCCCGAGCATTGTGGGCCTCATCATCTTCCTCCTGTGCACCCTCTTCATCAGTCTGCGCT
CCTCAGACCACCGGCAGGTGAACAGCCTGATGCAGACCGAGGAGTGCCCACCTATGCTAGACGCCACACAGCAGCAGCA
GCAGGTGGCAGCCTGTGAGGGCCGGGCCTTTGACAACGAGCAGGACGGCGTCACCTACAGCTACTCCTTCTTCCACTTC
TGCCTGGTGCTGGCCTCACTGCACGTCATGATGACGCTCACCAACTGGTACAAGTGCGTAGAGACCCGGAAGATGATCA
GCACGTGGACCGCCGTGTGGGTGAAGATCTGTGCCAGCTGGGCAGGGCTGCTCCTCTACCTGTGGACCCTGGTAGCCCC
ACTCCTCCTGCGCAACCGCGACTTCAGCTGA

A disclosed NOV9b polypeptide (SEQ ID NO:48) encoded by SEQ ID NO:47 is presented using the one-letter amino acid code in Table 9F. NOV9b amino acid changes, if any, are underlined in Table 9F.

TABLE 9F
Encoded NOV9b protein sequence.

(SEQ ID NO:48)

MGACLGACSLLSCVSPAGCASCLCGSAPCILCSCCPASRNSTVSRLIFTFFLFLGVLVSIIMLSPGVESQLYKLPWVCEEGAGIP
TVLQGHIDCGSLLGYRAVYRMCFATAAFFFFFTLLMLCVSSSRDPRAAIQNGPWFFKFLILVGLTVGAFYIPDGSFTNIWFYFGV
VGSFLFILIQLVLLIDFAHSWNQRWLGKAEECDSRAWYASLSSSTCLSIAAVALMFMYYTEPSGCHEGKVFISLNLTFCVCVSIA
AVLPKVQVSLPNSGLLQASVITLYTMFVTWSALSSIPEQKCNPHLPTQLGNETVVAGPEGYETQWWDAPSIVGLIIFLLCTLFIS
LRSSDHRQVNSLMQTEECPPMLDATQQQQQVAACEGRAFDNEQDGVTYSYSFFHFCLVLASLHVMMTLTNWYKCVETRKMISTWT
AVWVKICASWAGLLLYLWTLVAPLLLRNRDFS

A disclosed NOV9c nucleic acid (also referred to as 13375405) is a variant of NOV9a, encodes a novel TMS-2-like protein, and is shown in Table 9G. NOV9c nucleotide changes are underlined in Table 9G.

TABLE 9G
NOV9c Nucleotide Sequence

(SEQ ID NO:49)

ATGGGGGCCTGCCTGGGAGCCTGCTCCCTGCTCAGCTGCGTGAGTCCTGCTGGCTGTGCGTCCTGCCTCTGCGGCTCTG
CCCCCTGCATCCTGTGCAGCTGCTGCCCCGCCAGCCGCAACTCCACCGTGAGCCGCCTCATCTTCACGTTCTTCCTCTT
CCTGGGGGTGTTGGTGTCCATCATTATGCTGAGCCCGGGCGTGGAGAGTCAGCTCTACAAGCTGCCCTGGGTGTGTGAG
GAGGGGGCCGGGGATCCCCACCGTCCTGCAGGGCCACATCGACTGTGGCTCCCTGCTTGGCTACCGCGCTGTCTACCGA
TGTGCTTCGCCACGGCGGCCTTCTTCTTCTTTTTCACCCTGCTCATGCTCTGCGTGAGCAGCAGCCGGGACCCCCGGGC
TGCCATCCAGAATGGGTTTTGGTTCTTTAAGTTCCTGATCCTGGTGGGCCTCACCGTGGGTGCCTTCTACATTCCTGAC
GGCTCCTTCACCAACATCTGGTTCTACTTCGGCGTCGTGGGCTCCTTCCTCTTCATCCTCATCCAGCTGGTGCTGCTCA
TCGACTTTGCGCACTCCTGGAACCAGCGGTGGCTGGGCAAGGCCGAGGAGTGCGATTCCCGTGCCTGGTACGCATCACT
CTCCTCTTCTACTTGTCCGTCGATCGCGGCCGTGGCGCTGATGTTCATGTACTACACTGAGCCCAGCGGCTGCCACGAG
GGCAAGGTCTTCATCAGCCTCAACCTCACCTTCTGTGTCTGCGTGTCCATCGCTGCTGTCCTGCCCAAGGTCCAGGTGA
GCCTGCCTAACTCGGGTCTGCTGCAGGCCTCGGTCATCACCCTCTACACCATGTTTGTCACCTGGTCAGCCCTATCCAG
TATCCCTGAACAGAAATGCAACCCCCATTTGCCAACCCAGCTGGGCAACGAGACAGTTGTGGCAGGCCCCGAGGGCTAT
GAGACCCAGTGGTGGGATGCCCCGAGCATTGTGGGCCTCATCATCTTCCTCCTGTGCACCCTCTTCATCAGTCTGCGCT
CCTCAGACCACCGGCAGGTGAACAGCCTGATGCAGACCGAGGAGTGCCCACCTATGCTAGACGCCACACAGCAGCAGCA
GCAGGTGGCAGCCTGTGAGGGCCGGGCCTTTGACAACGAGCAGGACGGCGTCACCTACAGCTACTCCTTCTTCCACTTC
TGCCTGGTGCTGGCCTCACTGCACGTCATGATGACGCTCACCAACTGGTACAAGTGCGTAGAGACCCGGAAGATGATCA
GCACGTGGACCGCCGTGTGGGTGAAGATCTGTGCCAGCTGGGCAGGGCTGCTCCTCTACCTGTGGACCCTGGTAGCCCC
ACTCCTCCTGCGCAACCGCGACTTCAGCTGA

A disclosed NOV9c polypeptide (SEQ ID NO:50) encoded by SEQ ID NO:49 is presented using the one-letter amino acid code in Table 9H. NOV9c amino acid changes, if any, are underlined in Table 9H.

TABLE 9H
Encoded NOV9c protein sequence.

(SEQ ID NO:50)

MGACLGACSLLSCVSPAGCASCLCGSAPCILCSCCPASRNSTVSRLIFTFFLFLGVLVSIIMLSPGVESQLYKLPWVCEEGAGIP
TVLQGHIDCGSLLGYRAVYRMCFATAAFFFFFTLLMLCVSSSRDPRAAIQNGFWFFKFLILVGLTVGAFYIPDGSFTNIWFYFGV
VGSFLFILIQLVLLIDFAHSWNQRWLGKAEECDSRAWYASLSSSTCPSIAAVALMFMYYTEPSGCHEGVFISLNLTFCVCVSIA
AVLPKVQVSLPNSGLLQASVITLYTMFVTWSALSSIPEQKCNPHLPTQLGNETVVAGPEGYETQWWDAPSIVGLIIFLLCTLFIS
LRSSDHRQVNSLMQTEECPPMLDATQQQQQVAACEGRAFDNEQDGVTYSYSFFHFCLVLASLHVMMTLTNWYKCVETRKMISTWT
AVWVKICASWAGLLLYLWTLVAPLLLRNRDFS

A disclosed NOV9d nucleic acid (also referred to as 13375404) is a variant of NOV9a, encodes a novel TMS-2-like protein, and is shown in Table 9I. NOV9d nucleotide changes are underlined in Table 9I.

TABLE 9I
NOV9d Nucleotide Sequence

(SEQ ID NO:51)

ATGGGGGCCTGCCTGGGAGCCTGCTCCCTGCTCAGCTGCGTGAGTCCTGCTGGCTGTGCGTCCTGCCTCTGCGGCTCTG
CCCCCTGCATCCTGTGCAGCTGCTGCCCCGCCAGCCGCAACTCCACCGTGAGCCGCCTCATCTTCACGTTCTTCCTCTT
CCTGGGGGTGTTGGTGTCCATCATTATGCTGAGCCCGGGCGTGGAGAGTCAGCTCTACAAGCTGCCCTGGGTGTGTGAG
GAGGGGGCCGGGATCCCCACCGTCCTGCAGGGCCACATCGACTGTGGCTCCCTGCTTGGCTACCGCGCTGTCTACCGCA
TGTGCTTCGCCACGGCGGCCTTCTTCTTCTTTTTCACCCTGCTCATGCTCTGCGTGAGCAGCAGCCGGGACCCCCGGGC
TGCCATCCAGAATGGGTTTTGGTTCTTTAAGTTCCTGATCCTGGTGGGCCTCACCGTGGGTGCCTTCTACATTCCTGAC
GGCTCCTTCACCAACATCTGGTTCTACTTCGGCGTCGTGGGCTCCTTCCTCTTCATCCTCATCCAGCTGGTGCTGCTCA
TCGACTTTGCGCACTCCTGGAACCAGCGGTGGCTGGGCAAGGCCGAGGAGTGCGATTCCCGTGCCTGGTACGCATCACT
CTCCTCTTCTACTTGTCTGTCGATCGAGCCGTGGCGCTGATGTTCATGTACTACACTGAGCCCAGCGGCTGCCACGAG
GGCAAGGTCTTCATCAGCCTCAACCTCACCTTCTGTGTCTGCGTGTCCATCGCTGCTGTCCTGCCCAAGGTCCAGGTGA
GCCTGCCTAACTCGGGTCTGCTGCAGGCCTCGGTCATCACCCTCTACACCATGTTTGTCACCTGGTCAGCCCTATCCAG
TATCCCTGAACAGAAATGCAACCCCCATTTGCCAACCCAGCTGGGCAACGAGACAGTTGTGGCAGGCCCCGAGGGCTAT
GAGACCCAGTGGTGGGATGCCCCGAGCATTGTGGGCCTCATCATCTTCCTCCTGTGCACCCTCTTCATCAGTCTGCGCT
CCTCAGACCACCGGCAGGTGAACAGCCTGATGCAGACCGAGGAGTGCCCACCTATGCTAGACGCCACACAGCAGCAGCA
GCAGGTGGCAGCCTGTGAGGGCCGGGCCTTTGACAACGAGCAGGACGGCGTCACCTACAGCTACTCCTTCTTCCACTTC
TGCCTGGTGCTGGCCTCACTGCACGTCATGATGACGCTCACCAACTGGTACAAGTGCGTAGAGACCCGGAAGATGATCA
GCACGTGGACCGCCGTGTGGGTGAAGATCTGTGCCAGCTGGGCAGGGCTGCTCCTCTACCTGTGGACCCTGGTAGCCCC
ACTCCTCCTGCGCAACCGCGACTTCAGCTGA

A disclosed NOV9d polypeptide (SEQ ID NO:52) encoded by SEQ ID NO:51 presented using the one-letter amino acid code in Table 9J. NOV9d amino acid changes, if any, are underlined in Table 9J.

TABLE 9J
Encoded NOV9d protein sequence.

(SEQ ID NO:52)

MGACLGACSLLSCVSPAGCASCLCGSAPCILCSCCPASRNSTVSRLIFTFFLFLGVLVSIIMLSPGVESQLYKLPWVCEEGAGIP
TVLQGHIDCGSLLGYRAVYRMCFATAAFFFFFTLLMLCVSSSRDPRAAIQNGFWFFKFLILVGLTVGAFYIPDGSFTNIWFYFGV
VGSFLFILIQLVLLIDFAHSWNQRWLGKAEECDSRAWYASLSSSTCLSIAAVALMFMYYTEPSGCHEGKVFISLNLTFCVCVSIA
AVLPKVQVSLPNSGLLQASVITLYTMFVTWSALSSIPEQKCNPHLPTQLQNETVVAGPEGYETQWWDAPSIVGLIIFLLCTLFIS
LRSSDHRQVNSLMQTEECPPMLDATQQQQQVAACEGRAFDNEQDGVTYSYSFFHFCLVLASLHVMMTLTNWYKCVETRKMISTWT
AVWVKICASWAGLLLYLWTLVAPLLLRNRDFS

A disclosed NOV9e nucleic acid (also referred to as 13375403) is a variant of NOV9a, encodes a novel TMS-2-like protein, and is shown in Table 9K. NOV9e nucleotide changes are underlined in Table 9K.

TABLE 9K
NOV9e Nucleotide Sequence

(SEQ ID NO:53)

| | |
|---|---|
| ATGGGGGCCTGCCTGGGAGCCTGCTCCCTGCTCAGCTGCGTGAGTCCTGCTGGCTGTGCGTCCTGCCTCTGCGGCTCTG | |
| CCCCCTGCATCCTGTGCAGCTGCTGCCCCGCCAGCCGCAACTCCACCGTGAGCCGCCTCATCTTCACGTTCTTCCTCTT | ~ |
| CCTGGGGGTGTTGGTGTCCATCATTATGCTGAGCCCGGGCGTGGAGAGTCAGCTCTACAAGCTGCCCTGGGTGTGTGAG | |
| GAGGGGGCCGGGATCCCCACCGTCCTGCAGGGCCACATCGACTGTGGCTCCCTGCTTGGCTACCGCGCTGTCTACCGCA | |
| TGTGCTTCGCCACGGCGGCCTTCTTCTTCTTTTTCACCCTGCTCATGCTCTGCGTGAGCAGCAGCCGGGACCCCCGGGC | |
| TGCCATCCAGAATGGGTTTTGGTTCTTTAAGTTCCTGATCCTGGTGGGCCTCACCGTGGGTGCCTTCTACATTCCTGAC | |
| GGCTCCTTCACCAACATCTGGTTCTACTTCGGCGTCGTGGGCTCCTTCCTCTTCATCCTCATCCAGCTGGTGCTGCTCA | |
| TCGACTTTGCGCACTCCTGGAACCAGCGGTGGCTGGGCAAGGCCGAGGAGTGCGATTCCCGTGCCTGGTACGCATCACT | |
| CTCCTCTTCTACTTGTCTGTCGATCGCGGCCGCGGCGCTGATGTTCATGTACTACACTGAGCCCAGCGGCTGCCACGAG | |
| GGCAAGGTCTTCATCAGCCTCAACCTCACCTTCTGTGTCTGCGTGTCCATCGCTGCTGTCCTGCCCAAGGTCCAGGTGA | |
| GCCTGCCTAACTCGGGTCTGCTGCAGGCCTCGGTCATCACCCTCTACACCATGTTTGTCACCTGGTCAGCCCTATCCAG | |
| TATCCCTGAACAGAAATGCAACCCCCATTTGCCAACCCAGCTGGGCAACGAGACAGTTGTGGCAGGCCCCGAGGGCTAT | |
| GAGACCCAGTGGTGGGATGCCCCGAGCATTGTGGGCCTCATCATCTTCCTCCTGTGCACCCTCTTCATCAGTCTGCGCT | |
| CCTCAGACCACCGGCAGGTGAACAGCCTGATGCAGACCGAGGAGTGCCCACCTATGCTAGACGCCACACAGCAGCAGCA | |
| GCAGGTGGCAGCCTGTGAGGGCCGGGCCTTTGACAACGAGCAGGACGGCGTCACCTACAGCTACTCCTTCTTCCACTTC | |
| TGCCTGGTGCTGGCCTCACTGCACGTCATGATGACGCTCACCAACTGGTACAAGTGCGTAGAGACCCGGAAGATGATCA | |
| GCACGTGGACCGCCGTGTGGGTGAAGATCTGTGCCAGCTGGGCAGGGCTGCTCCTCTACCTGTGGACCCTGGTAGCCCC | |
| ACTCCTCCTGCGCAACCGCGACTTCAGCTCA | |

A disclosed NOV9e polypeptide (SEQ ID NO:54) encoded by SEQ ID NO:53 is presented using the one-letter amino acid code in Table 9L. NOV9e amino acid changes, if any, are underlined in Table 9L.

TABLE 9L
Encoded NOV9e protein sequence.

(SEQ ID NO:54)

MGACLGACSLLSCVSPAGCASCLCGSAPCILCSCCPASRNSTVSRLIFTFFLFLGVLVSIIMLSPGVESQLYKLPWVCEEGAGIP
TVLQGHIDCGSLLGYRAVYRMCFATAAFFFFFTLLMLCVSSSRDPRAAIQNGFWFFKFLILVGLTVGAFYIPDGSFTNIWFYFGV
VGSFLFILIQLVLLEDFAHSWNQRWLGKAEECDSRAWYASLSSSTCLSIAAAALMFMYYTEPSGCHEGKVFISLNLTFCVCVSIA
AVLPKVQVSLPNSGLLQASVITLYTMFVTWSALSSIPEQKCNPHLPTQLGNETVVAGPEGYETQWWDAPSIVGLIIFLLCTLFIS
LRSSDHRQVNSLMQTEECPPMLDATQQQQQVAACEGRAFDNEQDGVTYSYSFFHFCLVLASLHVMMTLTNWYKCVETRKMISTWT
AVWVKICASWAGLLLYLWTLVAPLLLRNRDFS

The lactose permease is an integral membrane protein that cotransports H(+) and lactose into the bacterial cytoplasm (Green A L, et al.; J Biol Chem Jul. 28, 2000;275(30):23240-6). Previous work has shown that bulky substitutions at glycine 64, which is found on the cytoplasmic edge of transmembrane segment 2 (TMS-2), cause a substantial decrease in the maximal velocity of lactose uptake without significantly affecting the K(m) values (Jessen-Marshall, A. E., Parker, N. J., and Brooker, R. J. (1997) J. Bacteriol. 179, 2616-2622). In the current study, mutagenesis was conducted along the face of TMS-2 that contains glycine-64. Single amino acid substitutions that substantially changed side-chain volume at codons 52, 57, 59, 63, and 66 had little or no effect on transport activity, whereas substitutions at codons 49, 53, 56, and 60 were markedly defective and/or had lower levels of expression. According to helical wheel plots, Phe-49, Ser-53, Ser-56, Gln-60, and Gly-64 form a continuous stripe along one face of TMS-2. Several of the TMS-2 mutants (S56Y, S56L, S56Q, Q60A, and Q60V) were used as parental strains to isolate mutants that restore transport activity. These mutations were either first-site mutations or second-site suppressors in TMS-1, TMS-2, TMS-7 or TMS-11. A kinetic analysis showed that the suppressors had a higher rate of lactose transport compared with the corresponding parental strains. Overall, the results of this study are consistent with the notion that a face on TMS-2, containing Phe-49, Ser-53, Ser-56, Gln-60, and Gly-64, plays a critical role in conformational changes associated with lactose transport. We hypothesize that TMS-2 slides across TMS-7 and TMS-11 when the lactose permease interconverts between the C1 and C2 conformations. This idea is discussed within the context of a revised model for the structure of the lactose permease.

The protein similarity information, expression pattern, and map location for the NOV9 suggest that NOV9 may have important structural and/or physiological functions characteristic of the TMS-2 family. Therefore, the NOV9 nucleic acids and proteins of the invention are useful in potential therapeutic applications implicated in various diseases and disorders described below and/or other pathologies. For example, the NOV9 compositions of the present invention will have efficacy for treatment of patients suffering from Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, Endocrine dysfunctions, Diabetes, obesity, Growth and Reproductive disorders, Multiple sclerosis, Leukodystrophies, Pain, Neuroprotection and transporter disorders. The NOV9 nucleic acid encoding ITGA7-like protein, and the ITGA7-like protein of the invention, or fragments thereof, may further be useful in diagnostic applications, wherein the presence or amount of the nucleic acid or the protein are to be assessed.

NOV10

A disclosed NOV10 nucleic acid of 2295 nucleotides (also referred to AC073487_dal) encoding a novel UNC5 Receptor-like receptor protein is shown in Table 10A. An open reading frame was identified beginning with an ATG initiation codon at nucleotides 64-66 and ending with a TGA codon at nucleotides 2902-2904. Putative untranslated regions upstream from the initiation codon and downstream from the termination codon are underlined in Table 10A, and the start and stop codons are in bold letters.

TABLE 10A
NOV10 Nucleotide Sequence

(SEQ ID NO:55)

CGGCGAGACTGGGGCCAGGGAGACAGCCCTGGGGGAGAGGCGCCCGAACCAGGCCGCGGGAGCAATGGGGGC
CCGGAGCGGAGCTCGGGGCGCGCTGCTGCTGGCACTGCTGCTCTGCTGGGACCCGAGGCTGAGCCAAGCAG
GTAGGAAGCGATCGGGTGAGGTGCTCCCTGACTCCTTCCCGTCAGCGCCAGCAGAGCCGCTGCCCTACTTC
CTGCAGGAGCCACAGGACGCCTACATTGTGAAGAACAAGCCTGTGGAGCTCCGCTGCCGCGCCTTCCCCGC
CACACAGATCTACTTCAAGTGCAACGGCGAGTGGGTCAGCCAGAACGACCACGTCACACAGGAAGGCCTGG
ATGAGGCCACCCTGGGGGCGCGGGGCGGCCTGCGGGTGCGCGAGGTGCAGATCGAGGTGTCGCGGCAGCAG
GTGGAGGAGCTCTTTGGGCTGGAGGATTACTGGTGCCAGTGCGTGGCCTGGAGCTCCGCGGGCACCACCAA
GAGTCGCCGAGCCTACGTCCGCATCGCCTGTCTGCGCAAGAACTTCGATCAGGAGCCTCTGGGCAAGGAGG
TGCCCCTGGACCATGAGGTTCTCCTGCAGTGCCGCCCGCCGGAGGGGGTGCCTGTGGCCGAGGTGGAATGG
CTCAAGAATGAGGATGTCATCGACCCCACCCAGGACACCAACTTCCTGCTCACCATCGACCACAACCTCAT
CATCCGCCAGGCCCGCCTGTCGGACACTGCCAACTATACCTGCGTGGCCAAGAACATCGTGGCCAAACGCC
GGAGCACCACTGCCACCGTCATCGTCTACGTGAATGGCGGCTGGTCCAGCTGGGCAGAGTGGTCACCCTGC
TCCAACCGCTGTGGCCGAGGCTGGCAGAAGCGCACCCGGACCTGCACCAACCCCGCTCCACTCAACGGAGG
GGCCTTCTGCGAGGGCCAGGCATTCCAGAAGACCGCCTGCACCACCATCTGCCCAGTCGATGGGGCGTGGA
CGGAGTGGAGCAAGTGGTCAGCCTGCAGCACTGAGTGTGCCCACTGGCGTAGCCGCGAGTGCATGGCGCCC
CCACCCCAGAACGGAGGCCGTGACTGCAGCGGGACGCTGCTCGACTCTAAGAACTGCACAGATGGGCTGTG
CATGCAAAGTGAGCCTGTCCCCGCAGTGCTGGAGGCCTCAGGGGATGCGGCGCTGTATGCGGGGCTCGTGG
TGGCCATCTTCGTGGTCGTGGCAATCCTCATGGCGGTGGGGGTGGTGGTGTACCGCCGCAACTGCCGTGAC
TTCGACACAGACATCACTGACTCATCTGCTGCCCTGACTGGTGGTTTCCACCCCGTCAACTTTAAGACGGC
AAGGCCCAGTAACCCGCAGCTCCTACACCCCTCTGTGCCTCCTGACCTGACAGCCAGCGCCGGCATCTACC
GCGGACCCGTGTATGCCCTGCAGGACTCCACCGACAAAATCCCCATGACCAACTCTCCTCTGCTGGACCCC
TTACCCAGCCTTAAGGTCAAGGTCTACAGCTCCAGCACCACGGGCTCTGGGCCAGGCCTGGCAGATGGGGC
TGACCTGCTGGGGGTCTTGCCGCCTGGCACATACCCTAGCGATTTCGCCCGGGACACCCACTTCCTGCACC
TGCGCAGCGCCAGCCTCGGTTCCCAGCAGCTCTTGGGCCTGCCCCGAGACCCAGGGAGCAGCGTCAGCGGC
ACCTTTGGCTGCCTGGGTGGGAGGCTCAGCATCCCCGGCACAGGTGTCAGCTTGCTGGTGCCCAATGGAGC
CATTCCCCAGGGCAAGTTCTACGAGATGTATCTACTCATCAACAAGGCAGAAAGTACCCTGCCGCTTTCAG
AAGGGACCCAGACAGTATTGAGCCCCTCGGTGACCTGTGGACCCACAGGCCTCCTGCTGTGCCGCCCCGTC
ATCCTCACCATGCCCCACTGTGCCGAAGTCAGTGCCCGTGACTGGATCTTTCAGCTCAAGACCCAGGCCCA
CCAGGGCCACTGGGAGCAGGAGGTGGTGACCCTGGATGAGGAGACCCTGAACACACCCTGCTACTGCCAGC
TGGAGCCCAGGGCCTGTCACATCCTGCTGGACCAGCTGGGCACCTACGTGTTCACGGGCGAGTCCTATTCC
CGCTCAGCAGTCAAGCGGCTCCAGCTGGCCGTCTTCGCCCCCGCCCTCTGCACCTCCCTGGAGTACAGCCT
CCGGGTCTACTGCCTGGAGGACACGCCTGTAGCACTGAAGGAGGTGCTGGAGCTGGAGCGGACTCTGGGCG
GATACTTGGTGGAGGAGCCGAAACCGCTAATGTTCAAGGACAGTTACCACAACCTGCGCCTCTCCCTCCAT
GACCTCCCCCATGCCCATTGGAGGAGCAAGCTGCTGGCCAAATACCAGGAGATCCCCTTCTATCACATTTG
GAGTGGCAGCCAGAAGGCCCTCCACTGCACTTTCACCCTGGAGAGGCACAGCTTGGCCTCCACAGAGCTCA
CCTGCAAGATCTGCGTGCGGCAAGTGGAAGGGGAGGGCCAGATATTCCAGCTGCATACCACTCTGGCAGAG
ACACCTGCTGGCTCCCTGGACACTCTCTGCTCTGCCCCTGGCAGCACTGTCACCACCCAGCTGGGACCTTA
TGCCTTCAAGATCCCACTGTCCATCCGCCAGAAGATATGCAACAGCCTAGATGCCCCCAACTCACGGGGCA
ATGACTGGCGGATGTTAGCACAGAAGCTCTCTATGGACCGGTACCTGAATTACTTTGCCACCAAAGCGAGC
CCCACGGGTGTGATCCTGGACCTCTGGGAAGCTCTGCAGCAGGACGATGGGGACCTCAACAGCCTGGCGAG
TGCCTTGGAGGAGATGGGCAAGAGTGAGATGCTGGTGGCTGTGGCCACCGACGGGGACTGCTGAGCCTCCT
GGGACAGCGGGCTGGCAGGGACTGGCAGGAGGCAGGTGCAGGGAGGCCTGGGGCAGCCTCCTGATGGGGAT
GTTTGGCCTCTGC

The disclosed NOV10 nucleic acid sequence, localized to chromosome 10, has 2213 of 2841 bases (77%) identical to a 2838 bp Rattus norvegicus transmembrane receptor UNCH2 mRNA (GENBANK-ID: RNU87306) (E=0.0).

A disclosed NOV10 polypeptide (SEQ ID NO:56) encoded by SEQ ID NO:55 is 946 amino acid residues and is presented using the one-letter amino acid code in Table 10B. Signal P, Psort and/or Hydropathy results predict that NOV10 does not contain a signal peptide and is likely to be localized at the plasma membrane with a certainty of 0.5140. The most likely cleavage site for a NOV10 peptide is between amino acids 26 and 27, at: SGA-GR.

TABLE 10B
Encoded NOV10 protein sequence.

(SEQ ID NO:56)

MGARSGARGALLLALLLCWDPRLSQAGRKRSGEVLPDSFPSAPAEPLPYFLQEPQDAYIVKNKPVELRCRA
FPATQIYFKCNGEWVSQNDHVTQEGLDEATLGARGGLRVREVQIEVSRQQVEELFGLEDYWCQCVAWSSAG
TTKSRRAYVRIACLRKNFDQEPLGKEVPLDHEVLLQCRPPEGVPVAEVEWLKNEDVIDPTQDTNFLLTIDH
NLIIRQARLSDTANYTCVAKNIVAKRRSTTATVIVYVNGGWSSWAEWSPCSNRCGRGWQKRTRTCTNPAPL
NGGAFCEGQAFQKTACTTICPVDGAWTEWSKWSACSTECAHWRSRECMAPPPQNGGRDCSGTLLDSKNCTD
GLCMQSEPVPAVLEASGDAALYAGLVVAIFVVVAILMAVGVVVYRRNCRDFDTDITDSSAALTGGFHPVNF
KTARPSNPQLLHPSVPPDLTASAGIYRGPVYALQDSTDKIPMTNSPLLDPLPSLKVKVYSSSTTGSGPGLA
DGADLLGVLPPGTYPSDFARDTHFLHLRSASLGSQQLLGLPRDPGSSVSGRFGCLGGRLSIPSTSVSLLVP
NGAIPQGKFYEMYLLINKAESTLPLSEGTQTVLSPSVTCGPTGLLLCRPVILTMPHCAEVSARDWIFQLKT
QAHQGHWEQEVVTLDEETLNTPCYCQLEPRACHILLDQLGTYVFTGESYSRSAVKRLQLAVFAPALCTSLE
YSLRVYCLEDTPVALKEVLELERTLGGYLVEEPKPLMFKDSYHNLRLSLHDLPHAHWRSKLLAKYQEIPFY
HIWSGSQKALHCTFTLERHSLASTELTCKICVRQVEGEGQIFQLHTTLAETPAGSLDTLCSAPGSTVTTQL
GPYAFKIPLSIRQKICNSLDAPNSRGNDWRMLAQKLSMDRYLNYFATKASPTGVILDLWEALQQDDGDLNS
LASALEEMGKSEMLVAVATDGDC

The NOV10 amino acid sequence has 860 of 946 amino acid residues (90%) identical to, and 893 of 946 amino acid residues (94%) similar to, the Rattus norvegicus 945 amino acid residue transmembrane receptor UNCH2 mRNA (ACC:008722)(E=0.0). The global sequence homology is 93.617% amino acid homology and 91.383% amino acid identity.

NOV10 is expressed in at least the following tissues: Respiratory System, Lung; Urinary System, Kidney; Gastro-intestinal/Digestive System, Liver, Small Intestine; Whole Organism; Female Reproductive System, Placenta, Chorionic Villus. In addition, the sequence is predicted to be expressed in the following tissues because of the expression pattern of (GENBANK-ID: ACC:008722) Transmembrane Receptor UNC5H2 homolog in species Rattus norvegicus: Respiratory System, Lung; Urinary System, Kidney; Gastro-intestinal/Digestive System, Liver, Small Intestine; Whole Organism; Female Reproductive System, Placenta, Chorionic Villus.

The disclosed NOV10 polypeptide has homology to the amino acid sequences shown in the BLASTP data listed in Table 10C.

TABLE 10C
BLAST results for NOV10

| Gene Index/ | | Length | Identity | Positives | |
|---|---|---|---|---|---|
| Identifier | Protein/Organism | (aa) | (%) | (%) | Expect |
| gi\|6678505\|ref\|NP_{—} | UNC-5 homolog | 931 | 597/910 | 707/910 | 0.0 |
| 033498.1\| | (C. elegans) 3 | | (65%) | (77%) | |
| | [Mus musculus] | | | | |
| gi\|4507837\|ref\|NP_{—} | unc5 (C. elegans | 931 | 585/910 | 702/910 | 0.0 |
| 003719.1\| | homolog) c; | | (64%) | (76%) | |
| | homolog of | | | | |
| | C. elegans | | | | |
| | transmembrane | | | | |
| | receptor Unc5 | | | | |
| | [Homo sapiens] | | | | |
| gi\|12857776\|dbj\| | putative | 945 | 861/951 | 899/951 | 0.0 |
| BAB31108.1\| | [Mus musculus] | | (90%) | (93%) | |
| (AK018177) | | | | | |
| gi\|11559982\|ref\|NP_{—} | transmembrane receptor | 945 | 860/951 | 893/951 | 0.0 |
| 071543.1\| | Unc5H2 | | (90%) | (93%) | |
| | [Rattus norvegicus] | | | | |
| gi\|15296526\|ref\|XP_{—} | unc5 (C. elegans | 931 | 586/910 | 703/910 | 0.0 |
| 042940.2\| | homolog) c | | (64%) | (76%) | |
| | [Homo sapiens] | | | | |

The homology between these and other sequences is shown graphically in the ClustalW analysis shown in Table 10D.

[image]

[image]

[image]

[image]

Table 10E-I lists the domain description from DOMAIN analysis results against NOV10. This indicates that the NOV10 sequence has properties similar to those of other proteins known to contain these domains.

TABLE 10E
Domain Analysis of NOV10

gln|Smart smart00218, ZU5, Domain present in ZO-l and Unc5-like netrin
receptors; Domain of unknown function. (SEQ ID NO:126)
Length=104 residues, 100.0% aligned
Score=149 bits (376), Expect=7e-37

| | | | |
|---|---|---|---|
| NOV10 | 541 | PGSSVSGTFGCLGGRLSIPGTGVSLLVPNGAIPQGKFYENYLLINKAESTLPLSEGTQTV | 600 |
| | | \| \|\|\|\|\| \|\|\|\| \| \|\|\| \|++\| \|\|\|\|\|\| \| \|\|+++ \|\| \| \| +\|+ | |
| 00218 | 1 | PSFLVSGTFDARGGRLRGPRTGVRLIIPPGAIPQGTRYTCTLVVHDKLSTPPPLEEGETL | 60 |
| NOV10 | 601 | LSPSVTCGPTGLLLCRPVILTMPHCAEVSARDWIFQLKTQAHQG 644 | |
| | | \|\|\| \| \|\|\| \| \| \|\|\|\|\| +\|\|\|\| + \|\|\| \| + \| | |
| 00218 | 61 | LSPVVECGPHGALFLRPVILEVPHCASLRPRDWEIVLLRSENGG 104 | |

TABLE 10F
Domain Analysis of NOV10

gln|Pfam|pfam00791, ZU5, ZU5 domain. Domain present in ZO-1 and Unc5-
like netrin receptors Domain of unknown function. (SEQ ID NO:127)
Length=104 residues, 100.0% aligned
Score=147 bits (371), Expect 3e-36

| | | | |
|---|---|---|---|
| NOV10 | 541 | PGSSVSGTFGCLGGRLSIPGRGVSLLVPNGAIPQGKFYEMYLLINKAESTLPLSEGTQTV | 600 |
| | | \| \|\|\|\|\| \|\|\|\| \| \|\|\| \|++\| \|\|\|\|\|\| \| \|\|+++ \|\| \| \| +\|+ | |
| 00791 | 1 | SGFLVSGTFDARGGRLRGPRTGVRLIIPPGAIPQGTRYTCYLVVHDKLSTPPPLEEGETL | 60 |
| NOV10 | 601 | LSPSVTCGPTGLLLCRPVILTMPHCAEVSARDWIFQLKTQAHQG 644 | |
| | | \|\|\| \| \|\|\| \| \| \|\|\|\|\| +\|\|\|\| + \|\|\| \| + \| | |
| 00791 | 61 | LSPVVECGPHGALFLRPVILEVPHCASLRPRDWELVLLRSENGG 104 | |

TABLE 10G
Domain Analysis of NOV10

gln|Smart smart00005, DEATH, DEATH domain, found in proteins involved
in cell death (apoptosis).; Alpha-helical domain present in a variety
of proteins with apoptotic functions. Some (but not all) of these
domains form homotypic and heterotypic dimers. (SEQ ID NO:128)
Length=96 residues, 99.0% aligned
Score=64.7 bits (156), Expect=2e-11

| | | | |
|---|---|---|---|
| NOV10 | 853 | GPYAFKIPLSIRQKICNSLDAPNSRGNDWRMLAQKLSM-DRYLNYFATKAL-----PTGV | 906 |
| | | \| \| + \|+\|+ \|\| + \|+\|\|\| \|\|+\|\| + + ++ \|++ + | |
| 00005 | 1 | PPGAASLTELTREKLAKLLD--HDLGDDWRELARKLGLSEADIDQIETESPRDLAEQSYQ | 58 |
| NOV10 | 907 | ILDLWEALQQDDGDLNSLASALEEMGKSEMLVAVATD 943 | |
| | | +\| \|\|\| + + \| +\| \|\| +\|\|+ + + + ++ | |
| 00005 | 59 | LLRLWEQREGKNATLGTLLEALRKMGRDDAVELLRSE 95 | |

TABLE 10H
Domain Analysis of NOV10

gnl|Smart smart00209, TSP1, Thrombospondin type 1 repeats; Type 1
repeats in thrombospondin-1 bind and activate TGF-beta. (SEQ ID
NO 129)
Length=51 residues, 100.0% aligned
Score=62.0 bits (149), Expect=1e-10

| | | | |
|---|---|---|---|
| NOV10 | 254 | WSSWAEWSPCSNRCGRGWQKRTRTCTNPAPLNGGAFCEGQAFQKTACTT-ICP | 305 |
| | | \| \|+\|\|\|\|\|\| \|\| \| \| \|\|\| \| \| \|\|\| \| \| + \|\| \|\| | |
| 00209 | 1 | WGEWSEWSPCSVTCGGGVQTRTRCCNPPP--NGGGPCTGPDTETRACNEQPCP | 51 |

TABLE 101
Domain Analysis of NOV10

gnl|Smart smart00409, IG. Immunoglobulin. (SEQ ID NO:130)
Length=86 residues, 100.0% aligned
Score=48.9 bits (115), Expect 1e-06

| | | | |
|---|---|---|---|
| NOV10 | 164 | PLGKEVPLDHEVLLQCRPPEGVPVAEVEWLKNEDVIDPTQDTNFLLTIDHN---LIIRQA | 220 |
| | | \| \| \| \| \| \| \| \| \| \| + + \| ++ \| \| | |
| 00409 | 1 | PPSVTVKEGESVTLSCEAS-GNPPPTVTWYKQGGKL-LAESGRFSVSRSGGNSTLTISNV | 58 |
| NOV10 | 221 | RLSDTANYTCVAKNIVAKRRSTTATVIVY 249 | |
| | | \|+ \|\|\| \| \| \| \| \|+ \| | |
| 00409 | 59 | TPEDSCTYTCAATNSSGSASSGT-TLTVL 86 | |

Migration of neurons from proliferative zones to their functional sites is fundamental to the normal development of the central nervous system. Mice homozygous for the rostral cerebellar malformation (rcm) mutation exhibit cerebellar and midbrain defects, apparently as a result of abnormal neuronal migration. Ackerman et al. (1997) reported that in rem-mutant mice, the cerebellum is smaller and has fewer folia than in wild type, ectopic cerebellar cells are present in midbrain regions by 3 days after birth, and there are abnormalities in postnatal cerebellar-neuronal migration. The authors isolated cDNAs encoding the rcm protein (Rcm). Sequence analysis revealed that the predicted 931-amino acid mouse protein is a transmembrane protein that contains 2 immunoglobulin (Ig)-like domains and 2 type I thrombospondin (THBS1; 188060) motifs in the extracellular region. Ig and THBS1 domains are also found in the extracellular region of the C. elegans UNC5 transmembrane protein, and the C-terminal 865-amino acid region of Rcm is 30% identical to UNC5. Ackerman et al. (1997) stated that the UNC5 protein is essential for dorsal guidance of pioneer axons and for the movement of cells away from the netrin ligand. In the developing brain of vertebrates, netrin-1 (601614) plays a role in both cell migration and axonal guidance. Leonardo et al. (1997) demonstrated that Rcm binds netrin-1 in vitro. Ackerman et al. (1997) concluded that Rcm and its ligand are important in critical migratory and/or cell-proliferation events during cerebellar development. Przyborski et al. (1998) found that disruption of the mouse rcm gene, also called the Unc5h3 gene, resulted in a failure of tangentially migrating granule cells to recognize the rostral boundary of the cerebellum.

By searching an EST database for sequences related to the Unc5h3 gene, Ackerman and Knowles (1998) identified a partial human fetal brain cDNA encoding UNC5C, the human Unc5h3 homolog. Using 5-prime RACE, they cloned a cDNA corresponding to the entire UNC5C coding region. The predicted 931-amino acid human protein has the overall domain structure of UNC5 family proteins, and is 97% identical to Unc5h3. Northern blot analysis revealed that the 9.5-kb UNC5 mRNA is expressed in brain and heart, and at low levels in kidney.

The protein similarity information, expression pattern, and map location for the NOV10 (UNC5 receptor-like) protein and nucleic acid disclosed herein suggest that NOV10 may have important structural and/or physiological functions characteristic of the UNC5 receptor family. Therefore, the NOV10 nucleic acids and proteins of the invention are useful in potential therapeutic applications implicated in various diseases and disorders described below and/or other pathologies. For example, the NOV10 compositions of the present invention will have efficacy for treatment of patients suffering from inflammatory and infectious diseases such as AIDS, cancer therapy, Neurologic diseases, Brain and/or autoimmune disorders like encephalomyelitis, neurodegenerative disorders, Alzheimer's Disease, Parkinson's Disorder, immune disorders, and hematopoietic disorders, endocrine diseases, muscle disorders, inflammation and wound repair, bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome and/or other pathologies and disorders. The NOV10 nucleic acid encoding UNC5 Receptor-like protein, and the UNC5 Receptor-like protein of the invention, or fragments thereof, may further be useful in diagnostic applications, wherein the presence or amount of the nucleic acid or the protein are to be assessed.

NOV11

NOV11 includes three novel Hepatocyte Growth Factor-like proteins disclosed below. The disclosed proteins have been named NOV11a, NOV11b and NOV11c.

NOV11a

A disclosed NOV 11a nucleic acid of 1782 nucleotides (also referred to GMba446g13_A) encoding a novel TMS-2-like protein is shown in Table 11A. An open reading frame was identified beginning with an ATG initiation codon at nucleotides 22-24 and ending with a TGA codon at nucleotides 1723-1725. Putative untranslated regions upstream from the initiation codon and downstream from the termination codon are underlined in Table 11A, and the start and stop codons are in bold letters.

TABLE 11A
NOV11a Nucleotide Sequence

(SEQ ID NO:57)

CAGGGCAGCGCTCGCCATTGAATGACTTCCAGGTGCTCCGGGGCACAGAGCTACCTGCTACATGCGGTGGT
GCCTGGGCCTTGGCAGGAGGATGTGGCAGATGCTGAAGAGTGTGCTGGTCGCTGTGGGCCCTTAACGGACT
GCTGGGCCTTCCACTACAATGTGAGCAGCCATGGTTGCCAACTGCTGCCATGGACTCAACACTCGCCCCAC
TCAAGGCTGTGGCATTCTGGGCGCTGTGACCTCTTCCAGAAGAAAGACTACATACGGACCTGCATCATGAA
CAATGGGGTTGGGTACCGGGGCACCATGGCCACGACCGTGGGTGGCCTGTCCTGCCAGGCTTGGAGCCACA
AGTTCCCGAATGATCACAAGTACATGCCCACGCTCCGGAATGGCCTGGAAGAGAACTTCTGCCATAACCCT
GATGGCGACCCCGGAGGTCCTTGGTGCCACACAACAGACCCTGCCGTGCGCTTCCAGAGCTGCGGCATCAA
ATCCTGCCGGGTGGCCGCGTGTGTCTGGTGCAATGGCGAGGAATACCGCGGCGCGGTAGACCGCACCGAGT
CAGGGCGCGAGTGCCAGCGCTGGGATCTTCAGCACCCGCACCAGCACCCCTTCGAGCCGGGCAGGTTCCTC
GACCAAGGTCTGGACGACAACTATTGCCGGAATCCTGACGGCTCCGAGCGGCCATGGTGCTACACTACGGA
TCCGCAGATCGAGCGAGAATTCTGTGACCTCCCCCGCTGCGGTTCCGAGGCACAGCCCCGCCAAGAGGCCA
CAAGTGTCAGCTGCTTCCGCGGGAAGGGTGAGGGCTACCGGGGCACAGCCAATACCACCACCGCGGGCGTA
CCTTGCCAGCGTTGGGACGCGCAAATCCCGCATCAGCACCGATTTACGCCAGAAAAATACGCGTGCAAGGA
CCTTCGGGAGAACTTCTGCCGGAACCTCGACGGCTCAGAGGCGCCCTGGTGCTTCACACTGCGGCCCGGCA
TGCGCGTGGGCTTTTGCTACCAGATCCGGCGTTGTACAGACGACGTGCGGCCCCAGGACTGCTACCACGGC
GCGGGGGAGCAGTACCGCGGCACGGTCAGCAAGACCCGCAAGGGTGTCCAGTGCCAGCGCGCGTCCGCTGA
GACGCCGCACAAGCCGCAGTTCACGTTTACCTCCGAACCGCATGCACAACTGGAGGAGAACTTCTGCCAGA
CCCCAGATGGGGATAGCCATGGGCCCTGGTGCTACACGATGGACCCAAGGACCCCATTCGACTACTGTGCC
CTGCGACGCTGCGCTGATGACCAGCCGCCATCAATCCTGGACCCCCCCGACCAGGTGCAGTTTGAGAAGTG
TGGCAAGAGGGTGGATCGGCTGGATCAGCGTCGTTCCAAGCTGCGCGTGGCTGGGGGCCATCCGGGCAACT
CACCCTGGACAGTCAGCTTGGGGAATCGGCAGGGCCAGCATTTCTGCGGGGGGTCTCTAGTGAAGGAGCAG
TGGATACTGACTGCCCGGCAGTGCTTCTCCTCCCAGCATATGCCTCTCACGGGCTATGAGGTATGGTTGGG
CACCCTGTTCCAGAACCCAGAACATGGAGAGCCAGGCCTACAGCGGGTCCCAGTAGCCAAGATCGTGTGTG
GGCCCTCAGGCTCCCAGCTTGTCCTGCTCAAGCTGGAGAGGTCTGTGACCCTGAACCAGCGTGTGGCCCTG
ATCTGCCTGCCGCCTGAATGATATGTGGTGCCTCCAGGGACCAAGTGTGAGATTGCAGGCCGGGGTGAGAC
CAAAGGT

The disclosed NOV11a nucleic acid sequence, localized to chromosome 1, has 1735 of 1787 bases (97%) identical to a Homo sapiens Macrophage Stimulating Protein mRNA (GENBANK-ID: RNU87306) (E=0.0).

A disclosed NOV11a polypeptide (SEQ ID NO:58) encoded by SEQ ID NO:57 is 567 amino acid residues and is presented using the one-letter amino acid code in Table 11B. Signal P, Psort and/or Hydropathy results predict that NOV11a does not contain a signal peptide and is likely to be localized to the peroxisome (microbody) with a certainty of 0.4531 and to the cytoplasm with a certainty of 0.4500. NOV11a is similar to the hepatocyte growth factor family, some members of which are released extracellularly. Therefore it is likely that NOV11a is available at the same sub-cellular localization and hence accessible to a diagnostic probe and for various therapeutic applications

TABLE 11B
Encoded NOV11a protein sequence.

(SEQ ID NO:58)

MTSRCSGAQSYLLHAVVPGPWQEDVADAEECAGRCGPLTDCWAFHYNVSSHGCQLLPWTQHSPHSRLWHSG
RCDLFQKKDYIRTCIMNNGVGYRGTMATTVGGLSCQAWSHKFPNDHKYMPTLRNGLEENFCHNPDGDPGGP
WCHTTDPAVRFQSCGIKSCRVAACVWCNGEEYRGAVDRTESGRECQRWDLQHPHQHPFEPGRFLDQGLDDN
YCRNPDGSERPWCYTTDPQIEREFCDLPRCGSEAQPRQEATSVSCFRGKGEGYRGTANTTTAGVPCQRWDA
QIPHQHRFTPEKYACKDLRENFCRNLDGSEAPWCFTLRPGMRVGFCYQIRRCTDDVRPQDCYHGAGEQYRG
TVSKTRKGVQCQRASAETPHKPQFTFTSEPHAQLEENFCQTPDGDSHGPWCYTMDPRTPFDYCALRRCADD
QPPSILDPPDQVQFEKCGKRVDRLDQRRSKLRVAGGHPGNSPWTVSLGNRQGQHFCGGSLVKEQWILTARQ
CFSSQHMPLTGYEVWLGTLFQNPQHGEPGLQRVPVAKMLCGPSGSQLVLLKLERSVTLNQRVALICLPPE

The NOV11a amino acid sequence has 249 of 456 amino acid residues (54%) identical to, and 552 of 567 amino acid residues (97%) identical to, and 556 or 567 amino acid residues (98%) similar to, the Homo sapiens 567 amino acid residue Hepatoctye Growth Factor protein (Q13208) (E=0.0). The global sequence homology is 97.707% amino acid homology and 97.354% amino acid identity.

NOV11a is expressed in at least the following tissues: lung, liver, kidney, brain. In addition, NOV11a is predicted to be expressed in the following tissues because of the expression pattern of a closely related Bos taurus Growth Factor homolog in species (GENBANK-ID: AW657716): lymph node, ovary, fat, hypothalamus, and pituitary.

NOV11a also has homology to the amino acid sequences shown in the BLASTP data listed in Table 11C.

TABLE 11C
BLAST results for NOV11a

| Gene Index/Identifier | Protein/Organism | Length (aa) | Identity (%) | Positives (%) | Expect |
|---|---|---|---|---|---|
| gi\|1141775 gb\| | hepatocyte growth | 567 | 552/567 | 556/567 | 0.0 |
| AAC63092.1 (U28054) | factor-like | | (97%) | (97%) | |
| | protein homolog | | | | |
| | [Homo sapiens] | | | | |
| gi\|123114 sp P26927 | HEPATOCYTE GROWTH | 771 | 532/557 | 540/557 | 0.0 |
| HGFL_HUMAN | FACTOR-LIKE | | (95%) | (96%) | |
| | PROTEIN PRECURSOR | | | | |
| | (MACROPHAGE | | | | |
| | STIMULATORY | | | | |
| | PROTEIN) (MSP) | | | | |
| | [Homo sapiens] | | | | |
| gi\|10337615\|ref\|NP_{—} | macrophage | 711 | 532/557 | 540/557 | 0.0 |
| 066278.1\| | stimulating 1 | | (95%) | (96%) | |
| | (hepatocyte | | | | |
| | growth factorlike) | | | | |
| | [Homo sapiens] | | | | |
| gi\|15294659\|ref\|XP_{—} | macrophage | 711 | 532/557 | 540/557 | 0.0 |
| 054070.1\| | stimulating 1 | | (95%) | (96%) | |
| | (hepatocyte | | | | |
| | growth factorlike) | | | | |
| | [Homo sapiens] | | | | |
| gi\|90615\|pir \|A40332 | macrophage- | 716 | 435/565 | 479/565 | 0.0 |
| | stimulating | | (76%) | (83%) | |
| | protein 1 | | | | |
| | precursor | | | | |
| | [Mus musculus] | | | | |

The homology of these sequences is shown graphically in the ClustalW analysis shown in Table 11D.

[image]

[image]

[image]

Table 11E-J lists the domain description from DOMAIN analysis results against NOV11a. This indicates that the NOV11a sequence has properties similar to those of other proteins known to contain these domains.

TABLE 11E
Domain Analysis of NOV11a

gnl|pfam|pfam00051, kringle, Kringle domain. Kringle domains have been
found in plasminogen, hepatocyte growth factors, prothrombin, and
apolipoprotein A. Structure is disuifide-rich, nearly all-beta. (SEQ
ID NO:136)
Length=79 residues, 100.0% aligned
Score=114 bits (284), Expect=2e-26

| | | | |
|---|---|---|---|
| NOV11a | 166 | CVWCNGEEYRGAVDRTESGRECQRWDLQHPHQHPF-EPGRFLDQGLDDNYCRNPDGSERP | 224 |
| \| \|\|\| \|\|\| \|\|\|\| \|\|\|\|\| \| \|\|+\| \| \|+ +\|\| +\|\|\|\|\|\|\|\| \|\|\| | | | |
| 000511 | 1 | CYHGNGENYRGTASTTESGAPCQRWDSQTPHRHSKYTPERYPAKGLGENYCRNPDGDERP | 60 |
| NOV11a | 225 | WCYTTDPQIEREFCDLPRC 243 | |
| | | \|\|\|\|\|\|\|++ \|+\|\|+\|\|\| | |
| 00051 | 61 | WCYTTDPRVRWEYCDIPRC 79 | |

TABLE 11F
Domain Analysis of NOV11a

gnl|Pfam|pfam00051, kringle, Kringle domain. (SEQ ID NO:137)
Length=79 residues, 100.0% aligned
Score=106 bits (264), Expect=4e-24

| | | | |
|---|---|---|---|
| NOV11a | 258 | CFRGKGEGYRGTANTTTAGVPCQRWDAQIPHQHRF-TPEKYACKDLRENFCRNLDGSEAP | 316 |
| | | \|+ \| \|\| \|\|\|\|\|+\|\| +\| \|\|\|\|\|\|+\| \|\|+\| \|\|\|+\| \| \| \|\|+\|\|\| \|\| \| \| | |
| 00051 | 1 | CYHGNGENYRGTASTTESGAPCQRWDSQTPHRHSKYTPERYPAKGLGENYCRNPDGDERP | 60 |
| NOV11a | 317 | WCFTLRPGMRVGFCYQIRRC 336 | |
| | | \|\|+\| \| +\| +\| \| \|\| | |
| 00051 | 61 | WCYTTDPRVRWEYC-DIPRC 79 | |

TABLE 11G
Domain Analysis of NOV11a

gln|Pfam|pfam00051, kringle, Eringle domain. (SEQ ID NO:138)
Length=79 residues, 100.0% aligned
Score=98.6 bits (244), Expect=9e-22

| | | | |
|---|---|---|---|
| NOV11a | 345 | CYHGAGEQYRGTVSKTRKGVQCQRASAETPHK-PQFTFTSEPHAQLEENFCQTPDGDSHG | 403 |
| | | \|\|\|\| \|\| \|\|\|\| \| \| \| \|\|\| ++\|\|\|+ ++\| \| \| \|\|+\|+ \|\|\|\| | |
| 000511 | 1 | CYHGNGENYRGTASTTESGAPCQRWDSQTPHRHSKYTPERYPAKGLGENYCRNPDGDER- | 59 |
| NOV11a | 404 | PWCYTMDPRTPFDYCALRRC 423 | |
| | | \|\|\|\|\| \|\|\| ++\|\| + \|\| | |
| 00051 | 60 | PWCYTTDPRVRWEYCDIPRC 79 | |

TABLE 11H
Domain Analysis of NOV11a

gnl|Pfam|pfam00051, kringle, Eringle domain. (SEQ ID NO:139)
Length=Th residues, 100.0% aligned
Score=94.4 bits (233), Expect2e-20

| | | | |
|---|---|---|---|
| NOV11a | 85 | CIMNNGVGYRGTMATTVGGLSCQAWSHKFPNDHKYM---PTLRNGLEENFCHNPDGDPGG | 141 |
| | | \| \|\| \|\|\|\| +\|\| \| \|\| \| + \|+ \| \|\| \|\|+\| \|\|\|\|\| | |
| 000511 | 1 | CYHGNGENYRGTASTTESGAPCQRWDSQTPHRHSKYTPERYPAKGLGENYCRNPDGDE-R | 59 |
| NOV11a | 142 | PWCHTTDPAVRFQSCGIKSC 161 | |
| | | \|\|\|+\|\|\|\| \|\|++ \| \| \| | |
| 00051 | 60 | PWCYTTDPRVRWEYCDIPRC 79 | |

TABLE 111
Domain Analysis of NOV11a

gln|Smart smart00130, KR, Kringle domain; Named after a Danish pastry.
Found in several serine proteases and in ROR-like receptors. Can occur
in up to 38 copies (in apolipoprotein(a)). Plasminogen-like kringles
possess affinity for free lysine and lysine- containing peptides. (SEQ
ID NO:140)
Length=83 residues, 97.6% aligned
Score=112 bits (280), Expect=Ee-26

| | | | |
|---|---|---|---|
| NOV11a | 166 | CVWCNGEEYRGAVDRTESGRECQRWDLQHPHQHPFEPGRFLDQGLDDNYCRNPDG-SERP | 224 |
| | | \|\|\|\|\|\|\|\|+\|\|+\|\|\|\|\|\|\|\|\|\|\|\|\|+\|\|+\|\|\|\|\|\|\|\|\|\|\| | |
| 00130 | 3 | CYAGNGESYRGTASTTKSGKPCQRWDSQTPHLHRFTPERFPELGLEHNYCRNPDGDSEGP | 62 |
| NOV11a | 225 | WCYTTDPQIEREFCDLPRCGS 245 | |
| | | \|\|\|\|\|\|\| + \|+\|\|+\|+\| \| | |
| 00130 | 63 | WCYTTDPNVRWEYCDIPQCES 83 | |

TABLE 11J
Domain Analysis of NOV11a

gnl|Smart smart00130, KR, Kringle domain; (SEQ ID NO:141)
Length=83 residues, 100.0% aligned
Score=108 bits (271), Expect=6e-25

| | | | |
|---|---|---|---|
| NOV11a | 343 | QDCYHGAGEQYRGTVSKTRKGVQCQRASAETPHKPQFTFTSEPHAQLEENFCQTPDGDSH | 402 |
| | | +\|\|\| \| \|\| \|\|\|\| \| \|+ \| \|\|\| ++\|\|\| +\|\| \| \|\| \|+\|+ \|\|\|\|\| | |
| 00130 | 1 | RDCYAGNGESYRGTASTTKSGDPCQRWDSQTPHLHRFTPERFPELGLEHNYCRNPDGDSE | 60 |
| NOV11a | 403 | GPWCYTMDPRTPFDYCALRRCAD 425 | |
| | | \|\|\|\|\|\| \|\| ++\|\| + +\| | |
| 00130 | 61 | GPWCYTTDPNVRWEYCDIPQCES 83 | |

Novel variants for the NOV11a nucleic acid and hepatocyte growth factor-like protein are also disclosed herein as variants of NOV11a. Variants, as described above, are reported individually, but any combination of all or a subset are also included.

A disclosed NOV11b nucleic acid (also referred to as cg34a.348) is a variant of NOV11a, encodes a novel hepatocyte growth factor-like protein, and is shown in Table 11K. NOV11b nucleotide changes are underlined in Table 11K.

TABLE 11K
NOV11b Nucleotide Sequence

(SEQ ID NO:59)

TGCAGCCTCCAGCCAGAAGGATGGGGTGGCTCCCACTCCTGCTGCTTCTGACTCAATGCTTAGGGGTCCCTGGGCAGCG
CTCGCCATTGAATGACTTCGAGGTGCTCCGGGGCACAGAGCTACAGCGGCTGCTACAAGCGGTGGTGCCCGGGCCTTGG
CAGGAGGATGTGGCAGATGCTGAAGAGTGTGCTGGTCGCTGTGGGCCCTTAATGGACTGCCGGGCGTTCCACTACAATG
TGAGCAGCCATGGTTGCCAACTGCTGCCATGGACTCAACACTCACCCCACACGAGGCTGCGGCATTCTGGGCGCTGTGA
CCTCTTCCAGGAGAAAGACTACATACGGACCTGCATCATGAACAATGGGGTTGGGTACCGGGGCACCATGGCCACGACC
GTGGGTGGCCTGTCCTGCCAGGCTTGGAGCCACAAGTTCCCGAACGATCACAGGTACATGCCCACGCTCCGGAATGGCC
TGGAAGAGAACTTCTGCCGTAACCCTGATGGCGACCCCGGAGGTCCTTGGTGCCACACAACAGACCCTGCCGTGCGCTT
CCAGAGCTGCGGCATCAAATCCTGCCGGTCTGCCGCGTGTGTCTGGTGCAATGGCGAGGAATACCGCGGCGCGGTAGAC
CGCACCGAGTCAGGGCGCGAGTGCCAGCGCTGGGATCTTCAGCACCCGCACCAGCACCCCTTCGAGCCGGGCAAGTACC
CCGACCAAGGTCTGGACGACAACTATTGCCGGAATCCTGACGGCTCCGAGCGGCCATGGTGCTACACTACGGATCCGCA
GATCGAGCGAGAATTCTGTGACCTCCCCCGCTGCGGTTCCGAGGCACAGCCCCGCCAAGAGGCCACAAGTGTCAGCTGC
TTCCGCGGGAAGGGTGAGGGCTACCGGGGCACAGCCAATACCACCACCGCGGGCGTACCTTGCCAGCGTTGGGACGCGC
AAATCCCGCATCAGCACCGATTTACGCCAGAAAAATACGCGTGCAAGGACCTTCGGGAGAACTTCTGCTGGAACCCCGA
CGGCTCAGAGGCGCCCTGGTGCTTCACACTGCGGCCCGGCATGCGCGTGGGCTTTTGCTACCAGATCCGGCGTTGTACA
GACGACGTGCGGCCCCAGGGTTGCTACCACGGCGCGGGGGAGCAGTACCGCGGCACGGTCAGCAAGACCCGCAAGGGTG
TCCAGTGCCAGCGCGCGTCCGCTGAGACGCCGCACAAGCCGCAGTTTACCTTTACCTCCGAACCGCATGCACAACTGGA
GGAGAACTTCTGCCGCGACCCAGATGGGGCTAGCTATGGGCCCTGGTGCTACACGATGGACCCAAGGACCCCATTCGAC
TACTGTGCCCTGCGACGCTGCGCTGATGACCAGCCGCCATCAATCCTGGACCCCCCCGACCAGGTGCAGTTTGAGAAGT
GTGGCAAGAGGGTGGATCGGCTGGATCAGCGTTGTTCCAAGCTGCGCGTGGCTGGGGGCCATCCGGGCAACTCACCCTG
GACAGTCAGCTTGCGGAATAGGCAGGGCCAGCATTTCTGCGGGGGGTCTCTAGTGAAGGAGCAGTGGATACTGACTGCC
CGGCAGTGCTTCTCCTCCAGCCATATGCCTCTCACGGGCTATGAGGTATGGTTGGGCACCCTGTTCCAGAACCCACAAC
ATGGAGAGCCAGGCCTACAGCGGGTCCCAGTAGCCAAGATGCTGTGTGGGCCCTCAGGCTCTCAGCTTGTCCTGCTCAA
GCTGGAGAGATCTGTGACCCTGAACCAGCGTGTGGCCCTGATCTGCCTGCCGCCTGAATGGTATGTGGTGCCTCCAGGG
ACCAAGTGTGAGATGGCAGGCCGGGGTGAGACCAAAGGTACGGGTAATGACACAGTCCTAAATGTGGCCTTGCTGAATG
TCATCTCCAACCAGGAGTGTAACATCAAGCACCGAGGACATGTGCGGGAGAGCGAGATGTGCACTGAGGGACTGTTGGC
CCCTGTGGGGGCCTGTGAGGGGGGTGACTACGGGGGCCCACTTGCCTGCTTTACCCACAACTGCTGGGTCCTGGAAGGA
ATTAGAATCCCCAACCGAGTATGCGCAAGGTCGCGCTGGCCAGCCGTCTTCACACGTGTCTCTGTGTTTGTGGACTGGA
TTCACAAGGTCATGAGACTGGGTTAGGCCCAGCCTTGACGCCATATGCTTTGGGGAGGACAAAACTT

A disclosed NOV11b polypeptide (SEQ ID NO:60) encoded by SEQ ID NO:59 is presented using the one-letter amino acid code in Table 11L. NOV11b amino acid changes, if any, are underlined in Table 11L.

TABLE 11L
Encoded NOV11b protein sequence.

(SEQ ID NO:60)

MGWLPLLLLLTQCLGVPGQRSPLNDFEVLRGTELQRLLQAVVPGPWQEDVADAEECAGRCGPLMDVRAFHYNVSSHGCQLLPWTQ
HSPHTRLRHSGRCDLFQEKDYIRTCIMNNGVGYRGTMATTVGGLSCQAWSHKFPNDHRYMPTLRNGLEENFCRNPDGDPGGPWCH
TTDPAVRFQSCGIKSCRSAACVWCNGEEYRGAVDRTESGRECQRWDLQHPHQHPFEPGKYPDQGLDDNYCRNPDGSERPWCYTTD
PQIEREFCDLPRCGSEAQPRQEATSVSCFRGKGEGYRGTANTTTAGVPCQRWDAQIPHQHRFTPEDYACKDLRENFCWNPDGSEA
PWCFTLRPGMRVGFCYQIRRCTDDVRPQGCYHGAGEQYRGTVSKTRKGVQCQRASAETPHKPQFTFTSEPHAQLEENFCRDPDGD
SYGPWCYTMDPRTPFDYCALRRCADDQPPSILDPPDQVQFEKCGKRVDRLDQRCSKLRVAGGHPGNSPWTVSLRNRQGQHFCGGS
LVKEQWILTARQCFSSSHMPLTGYEVWLGTLFQNPQHGEPGLQRVPVAKMLCGPSGSQLVLLKLERSVTLNQRVALICLPPEWYV
VPPGTKCEIAGRGETKGTGNDTVLNVALLNVISNQECNIKHRGHVRESEMCTEGLLAPVGACEGGDYGGPLACFTHNCWVLEGIR
IPNRVCARSRWPAVFTRVSVFVDWIHKVMRLG

A disclosed NOV11c nucleic acid (also referred to as cg34a.349) is a variant of NOV11a, encodes a novel hepatocyte growth factor-like protein, and is shown in Table 11M. NOV11c nucleotide changes are underlined in Table 11M.

TABLE 11M
NOV11c Nucleotide Sequence

(SEQ ID NO:61)

TGCAGCCTCCAGCCAGAAGGATGGGGTGGCTCCCACTCCTGCTGCTTCTGACTCAATGCTTAGGGGTCCCTGGGCAGCG
CTCGCCATTGAATGACTTCGAGGTGCTCCGGGGCACAGAGCTACAGCGGCTGCTACAAGCGGTGGTGCCCGGGCCTTGG
CAGGAGGATGTGGCAGATGCTGAAGAGTGTGCTGGTCGCTGTGGGCCCTTAATGGACTGCCGGGCGTTCCACTACAATG
TGAGCAGCCATGGTTGCCAACTGCTGCCATGGACTCAACACTCACCCCACACGAGGCTGCGGCATTCTGGGCGCTGTGA
CCTCTTCCAGGAGAAAGACTACATACGGACCTGCATCATGAACAATGGGGTTGGGTACCGGGGCACCATGGCCACGACC
GTGGGTGGCCTGTCCTGCCAGGCTTGGAGCCACAAGTTCCCGAACGATCACAGGTACATGCCCACGCTCCGGAATGGCC
TGGAAGAGAACTTCTGCCGTAACCCTGATGGCGACCCCGGAGGTCCTTGGTGCCACACAACAGACCCTGCCGTGCGCTT
CCAGAGCTGCGGCATCAAATCCTGCCGGTCTGCCGCGTGTGTCTGGTGCAATGGCGAGGAATACCGCGGCGCGGTAGAC
CGCACCGAGTCAGGGCGCGAGTGCCAGCGCTGGGATCTTCAGCACCCGCACCAGCACCCCTTCGAGCCGGGCAAGTACC
CCGACCAAGGTCTGGACGACAACTATTGCCGGAATCCTGACGGCTCCGAGCGGCCATGGTGCTACACTACGGATCCGCA
GATCGAGCGAGAATTCTGTGACCTCCCCCGCTGCGGTTCCGAGGCACAGCCCCGCCAAGAGGCCACAAGTGTCAGCTGC
TTCCGCGGGAAGGGTGAGGGCTACCGGGGCACAGCCAATACCACCACCGCGGGCGTACCTTGCCAGCGTTGGGACGCGC
AAATCCCGCATCAGCACCGATTTACGCCAGAAAAATACGCGTGCAAGGACCTTCGGGAGAACTTCTGCCGGAACCCCGA
CGGCTCAGAGGCGCCCTGGTGCTTCACCCTGCGGCCCGGCATGCGCGTGGGCTTTTGCTACCAGATCCGGCGTTGTACA
GACGACGTGCGGCCCCAGGGTTGCTACCACGGCGCGGGGGAGCAGTACCGCGGCACGGTCAGCAAGACCCGCAAGGGTG
TCCAGTGCCAGCGCGCGTCCGCTGAGACGCCGCACAAGCCGCAGTTTACCTTTACCTCCGAACCGCATGCACAACTGGA
GGAGAACTTCTGCCGCGACCCAGATGGGGATAGCTATGGGCCCTGGTGCTACACGATGGACCCAAGGACCCCATTCGAC
TACTGTGCCCTGCGACGCTGCGCTGATGACCAGCCGCCATCAATCCTGGACCCCCCCGACCAGGTGCAGTTTGAGAAGT
GTGGCAAGAGGGTGGATCGGCTGGATCAGCGTTGTTCCAAGCTGCGCGTGGCTGGGGGCCATCCGGGCAACTCACCCTG
GACAGTCAGCTTGCGGAATAGGCAGGGCCAGCATTTCTGCGGGGGGTCTCTAGTGAAGGAGCAGTGGATACTGACTGCC
CGGCAGTGCTTCTCCTCCAGCCATATGCCTCTCACGGGCTATGAGGTATGGTTGGGCACCCTGTTCCAGAACCCACAAC
ATGGAGAGCCAGGCCTACAGCGGGTCCCAGTAGCCAAGATGCTGTGTGGGCCCTCAGGCTCTCAGCTTGTCCTGCTCAA
GCTGGAGAGATCTGTGACCCTGAACCAGCGTGTGGCCCTGATCTGCCTGCCGCCTGAATGGTATGTGGTGCCTCCAGGG
ACCAAGTGTGAGATTGCAGGCCGGGGTGAGACCAAAGGTACGGGTAATGACACAGTCCTAAATGTGGCCTTGCTGAATG
TCATCTCCAACCAGGAGTGTAACATCAAGCACCGAGGACATGTGCGGGAGAGCGAGATGTGCACTGAGGGACTGTTGGC
CCCTGTGGGGGCCTGTGAGGGGGGTGACTACGGGGGCCCACTTGCCTGCTTTACCCACAACTGCTGGGTCCTGGAAGGA
ATTAGAATCCCCAACCGAGTATGCGCAAGGTCGCGCTGGCCAGCCGTCTTCACACGTGTCTCTGTGTTTGTGGACTGGA
TTCACAAGGTCATGAGACTGGGTTAGGCCCAGCCTTGACGCCATATGCTTTGGGGAGGACAAAACTT

A disclosed NOV11c polypeptide (SEQ ID NO:62) encoded by SEQ ID NO:61 is presented using the one-letter amino acid code in Table 11N. NOV11c amino acid changes, if any, are underlined in Table 11N.

TABLE 11N
Encoded NOV11c protein sequence.

(SEQ ID NO:62)

MGWLPLLLLLTQCLGVPGQRSPLNDFEVLRGTELQRLLQAVVPGPWQEDVADAEECAGRCGPLMDCRAFHYNVSSHGCQLLPWTQ
HSPHTRLRHSGRCDLFQEKDYIRTCIMNNGVGYRGTMATTVGGLSCQAWSHKFPNDHRYMPTLRNGLEENFCRKPDGDPGGPWCH
TTDPAVRFQSCGIKSCRSAACVWCNGEEYRGAVDRTESGRECQRWDLQHPHQHPFEPGKYPDQGLDDNYCRNPDGSERPWCYTTD
PQIEREFCDLPRCGSEAQPRQEATSVSCFRGKGEGYRGTANTTTAGVPCQRWDAQIPHQHRFTPEKYACKDLRENFCRNPDGSEA
PWCFTLRPGNRVGFCYQIRRCTDDVRPQGCYHGAGEQYRGTVSKTRKGVQCQRASAETPHKPQFTFTSEPHAQLEENFCRDPDGD
SYGPWCYTMDPRTPFDYCALRRCADDQPPSILDPPDQVQFEKCGKRVDRLDQRCSKLRVAGGHPGNSPWTVSLRNRQGQHFCGGS
LVKEQWILTARQCFSSSHMPLTGYEVWLGTLFQNPQHGEPGLQRVPVAKMLCGPSGSQLVLLKLERSVTLNQRVALICLPPEWYV
VPPGTKCEIAGRGETKGTGNDTVLNVALLNLISNQECNIKHRGHVRESEMCTEGLLAPVGACEGGDYGGPLACFTHNCWVLEGIR
IPNRVCARSRWPAVFTRVSVFVDWIHKVMRLG

In vitro, normal human melanocytes require synergistic mitogens, in addition to the common growth factors present in serum, in order to proliferate. The peptide growth factors that confer stimulation are fibroblast growth factors (such as bFGF/FGF2), hepatocyte growth factor/scatter factor (HGF/SF), mast/stem cell factor (M/SCF), endothelins (such as ET-1) and melanotropin (MSH). The proper function of these factors and their cognate receptors is likely to be important in vivo, as all five ligands are produced in the skin, and disruption of their normal function, by elimination due to deletions or mutations, or overproduction due to ectopic expression, disrupts the normal distribution of melanocytes. The synergistic growth factors activate intracellular signal transduction cascades and maintain the intermediate effectors at optimal levels and duration required for nuclear translocation and modification of transcription factors. The consequent induction of immediate-early response genes, such as cyclins, and subsequent activation of cyclin-dependent kinases (CDK4, CDK6 and CDK2) inactivates the retinoblastoma family of proteins (pRb, p107 and p130, together termed pocket proteins), and releases their suppressive association with E2F transcription factors. Molecular events that disrupt this tight control of pocket proteins and cause their inactivation, increase E2F transcriptional activity and confer autonomous growth on melanocytes. (10761990)

Organ culture and transplantation experiments in the early 1960s and 1970s have demonstrated that growth and morphogenesis of the epithelium of the mammary gland are controlled by mesenchymal-epithelial interactions. The identification of molecules that provide the essential signals exchanged in mesenchymal-epithelial interactions is an area of active research. Recent evidence suggests that morphogenic programs of epithelia can be triggered by mesenchymal factors that signal via tyrosine kinase receptors. This review concentrates on the effects of two mesenchymal factors, Hepatocyte Growth Factor/Scatter Factor and neuregulin, on morphogenesis and differentiation of mammary epithelial cells in vitro and signaling pathways involved during morphogenesis of mammary epithelial cells (10959405).

Increasing evidence indicates that HGF acts as a multifunctional cytokine on different cell types. This review addresses the molecular mechanisms that are responsible for the pleiotropic effects of HGF. HGF binds with high affinity to its specific tyrosine kinase receptor c-met, thereby stimulating not only cell proliferation and differentiation, but also cell migration and tumorigenesis. The three fundamental principles of medicine-prevention, diagnosis, and therapy-may be benefited by the rational use of HGF. In renal tubular cells, HGF induces mitogenic and morphogenetic responses. In animal models of toxic or ischemic acute renal failure, HGF acts in a renotropic and nephroprotective manner. HGF expression is rapidly up-regulated in the remnant kidney of nephrectomized rats, inducing compensatory growth. In a mouse model of chronic renal disease, HGF inhibits the progression of tubulointerstitial fibrosis and kidney dysfunction. Increased HGF mRNA transcripts were detected in mesenchymal and tubular epithelial cells of rejecting kidney. In transplanted patients, elevated HGF levels may indicate renal rejection. When HGF is considered as a therapeutic agent in human medicine, for example, to stimulate kidney regeneration after acute injury, strategies need to be developed to stimulate cell regeneration and differentiation without an induction of tumorigenesis. (10760078)

The protein similarity information, expression pattern, and map location for the NOV11 protein and nucleic acid suggest that NOV11 may have important structural and/or physiological functions characteristic of the hepatocyte growth factor family. Therefore, the NOV11 nucleic acids and proteins of the invention are useful in potential therapeutic applications implicated in various diseases and disorders described below and/or other pathologies. For example, the NOV11 compositions of the present invention will have efficacy for treatment of patients suffering from various diseases involving blood coagulation, and hepatocellualr carcinoma; cancers including but not limited to lung, breast and ovarian cancer; tumor suppression, senescence, growth regulation, modulation of apoptosis, reproductive control and associated disorders of reproduction, endometrial hyperplasia and adenocarcinoma, psychotic and neurological disorders, Alzheimer's disease, endocrine disorders, inflammatory disorders, gastrointestinal disorders and disorders of the respiratory system; hematopoiesis, immunotherapy, immunodeficiency diseases, all inflammatory diseases; cancer therapy; autoimmune diseases; obesity, modulation of myofibroblast development; applications to modulation of wound healing; potential applications to control of angiogenesis muscle disorders, neurologic diseases and/or other pathologies and disorders. The NOV11 nucleic acid encoding hepatocyte growth factor-like protein, and the hepatocyte growth factor-like protein of the invention, or fragments thereof, may further be useful in diagnostic applications, wherein the presence or amount of the nucleic acid or the protein are to be assessed.

NOV12

A disclosed NOV12 nucleic acid of 1407 nucleotides (also referred to GMAC023940_A) encoding a novel 26S protease regulatory subunit-like protein is shown in Table 12A. An open reading frame was identified beginning with an ATG initiation codon at nucleotides 58-60 and ending with a TGA codon at nucleotides 1377-1379. Putative untranslated regions upstream from the initiation codon and downstream from the termination codon are underlined in Table 12A, and the start and stop codons are in bold letters.

TABLE 12A
NOV12 Nucleotide Sequence

(SEQ ID NO:63)

ACTTTGAATCATCAACATAAAGAAAAAATGTTAAAAGCTCTCCCAGGCCAAGGCAAGATGGGTCAAAGTCA
GAGTGGTGGTCATGGTCCTGGAGGTGGCAAGAAGGATGACAAGGACAAGAAAAAGAAATATGAACCTCCTG
TACCAACTACAGTGGGGAAAAAGAAGAAGAAAACAAAGGGACCAGATGCTGCCAGCAAACTGCCACTGGTG
ACACCTCACACTCAGTGCCAGTTAAAATTACTGAAGTTAGAGAGAATTAAAGACTATCTTCTCATGGAGGA
AGAATTCATTAGAAATCAGGAACAAATGAAACCATTAGAAGAAAAGCAAGAAGGGAAAAGATCAAAAGTGG
ATGATCTGAGGGGGACCCCAATGTCAGTAGGAATCTTGGAAGAGATCATTGATGACAATCATGCCATCGTG
TCTACATCTGTGGGCTCAGAACACTACATCAGCATTCTTTCATTTGCAGACAAGGATCTTCTGGAACCTGG
CTGCTCGGTCAGGCTCAACCACAAGGTGCATACCATGATAGGGGTGCTGATGGATGACATGGATCCCCTGG
TCACAGTGATGAAGGTGGAAAAGGCCCCCCAAGAGACCTATGCAGATACTGGGGGGTTGGACAACCAAATT
CGGGAAATTAAGGAATCTGTGGAGCTTCCTCTCACCCATCCTGAATATTATGAAGAGATGGGTATAAAGCC
TCCAAAGGGGGTCATTCTCTGTGGTCCACCTGGCACAGGTAAAACCTTGTTAGCCAAAGCAGTAGCAAACC
AAACCTCAGCCACTTTCTTGAGAGTGGTTGGCTCTGAACTTATTCAGAAGTACCTAGGTGATGGGCCCAAA
CTCGGACGGGAATTGTTTCGAGTTGCTGAAGAACGTGCACCGTCCATTGTGTTTATTGATGAAATTGACGC
CATTGGGACAAAAAGATATGACTCCAATTCTGGTGGTGAGAGAGAAATTCAGCGAACAACGTTGGAACTGC
TGAACCAGTTGGATGGATTTGATTCTAGGGTAGATGTGAAAGCTATAATGGCCACAAACCAAATAGAAACT
TTGGATCCAGCGCTTATCAGACCAGGCCGCATTGGCAGGAAGATTGAGTTCCCCCTGCCTGATGAAAAGAC
GAAGAAGCCCATCTTTCAGATTCACACAAGCAGGATGACGCTGGCTGATGATGTAACCCTGCACGACTTGA
TCATGGCTAAAGATGACCTCTCTGGTGCTGACATCAAGGCAGTCTGTACAGAAGCTGGTCTGATGGCCTTA
AGAGAACGTAGAATGAAAGTAACAAATGAAGACTTCAAAAAATCTAAAGAAAATGTTCTTTATAAGAAACA
GGAAGACACCCCTGAGGGGCTGTATCTCATGTGAACTACGGCTGCCATCAGGAAAATG

The disclosed NOV12 nucleic acid sequence, localized to chromosome 12, has 1320 of 1362 bases (96%) identical to a Homo sapiens 26S Protease Regulatory Subunit 4 mRNA (GENBANK-ID: HUM26SPSIV) (E=8.6e⁻²⁸⁵).

A disclosed NOV12 polypeptide (SEQ ID NO:64) encoded by SEQ ID NO:63 is 440 amino acid residues and is presented using the one-letter amino acid code in Table 12B. Signal P, Psort and/or Hydropathy results predict that NOV12 does not contain a signal peptide and is likely to be localized in the nucleus with a certainty of 0.9800.

TABLE 12B
Encoded NOV12 protein sequence.

(SEQ ID NO:64)

MGQSQSGGHGPGGGKKDDKDKKKKYEPPVPTTVGKKKKKTKGPDAASKLPLVTPHTQCQLKLLKLERIKDY
LLMEEEFIRNQEQMKPLEEKQEGKRSKVDDLRGTPMSVGILEEIIDDNHAIVSTSVGSEHYISILSFADKD
LLEPGCSVRLNHKVHTMIGVLMDDMDPLVTVMKVEDAPQETYADTGGLDNQIREIKESVELPLTHPEYYEE
MGIKPPKGVILCGPPGTGKTLLAKAVANQTSATFLRVVGSELIQKYLGDGPKLGRELFRVAEERAPSIVFI
DEIDAIGTKRYDSNSGGEREIQRTTLELLNQLDGFDSRVDVKAIMATNQIETLDPALIRPGRIGRKIEFPL
PDEKTKKPIFQIHTSRMTLADDVTLHDLIMAKDDLSGADIKAVCTEAGLMALRERRMKVTNEDFKKSKENV
LYKKQEDTPEGLYL

The NOV12 amino acid sequence has 414 of 440 amino acid residues (94%) identical to, and 422 of 440 amino acid residues (95%) similar to, the 440 amino acid residue 26S Protease Regulatory Subunit 4 protein from Homo sapiens (Q03527) (E=6.3e⁻²¹⁸). The global sequence homology is 94.545% amino acid homology and 94.091% amino acid identity.

NOV12 is expressed in at least the following tissues: parathyroid-tumor, skin, Colon carcinoma, neuroepithelium, lung carcinoma, brain, liver, kidney, neuron, spleen, olfactory, T-cell, cartilage, ovary, heart. In addition, NOV12 is predicted to be expressed in the following tissues because of the expression pattern of a closely related Mus musculus 26S protease regulatory subunit homolog (GENBANK-ID: AM325227): parathyroid-tumor, skin, Colon carcinoma, neuroepithelium, lung carcinoma, brain, liver, kidney, neuron, spleen, olfactory, T-cell, cartilage, ovary, heart.

NOV12 also has homology to the amino acid sequences shown in the BLASTP data listed in Table 12C.

TABLE 12C
BLAST results for NOV12

| Gene Index/Identifier | Protein/Organism | Length (aa) | Identity (%) | Positives (%) | Expect |
|---|---|---|---|---|---|
| gi\|4506207\|ref\|NP_ | proteasome | 440 | 414/440 | 422/440 | 0.0 |
| 002793.1\| | (prosome,macropain) | | (94%) | (95%) | |
| | 26S subunit, | | | | |
| | ATPase, 1; | | | | |
| | Proteasome 26S | | | | |
| | subunit | | | | |
| | [Homo sapiens] | | | | |
| gi\|6679501\|ref\|NP_ | protease | 440 | 415/440 | 422/440 | 0.0 |
| 032973.1\| | (prosome, | | (94%) | (95%) | |
| | macropain) 26S | | | | |
| | subunit, ATPase 1 | | | | |
| | [Mus musculus] | | | | |
| gi\|345717\|pir \|A44468 | 26S proteasome | 440 | 413/440 | 421/440 | 0.0 |
| | regulatory chain | | (93%) | (94%) | |
| | 4 [validated] | | | | |
| | [Homo sapiens] | | | | |
| gi\|2492516\|sp \|Q90732 | 26S PROTEASE | 440 | 409/440 | 418/440 | 0.0 |
| PRS4_CHICK | REGULATORY | | (92%) | (94%) | |
| | SUBUNIT 4 (P26S4) | | | | |
| | [Gallus gallus] | | | | |
| gi\|7301070\|gb \| | Pros26.4 gene | 439 | 379/440 | 406/440 | 0.0 |
| AAF56205.1\| (AE003745) | product | | (86%) | (92%) | |
| | [Drosophila | | | | |
| | melanogaster] | | | | |

The homology of these sequences is shown graphically in the ClustalW analysis shown in Table 12D.

[image]

[image]

[image]

Table 12E and 12F lists the domain description from DOMAIN analysis results against NOV12. This indicates that the NOV12 sequence has properties similar to those of other proteins known to contain these domains.

TABLE 12E
Domain Analysis of NOV12

gnl|Pfam|pfam00004, AAA, ATPase family associated with various
cellular activities (AAA). AAA family proteins often perform
chaperone-like functions that assist in the assembly, operation, or
disassembly of protein complexes. (SEQ ID NO:147)
Length=186 residues, 100.0% aligned
Score=200 bits (509), Expect=1e-52

| | | | |
|---|---|---|---|
| NOV12 | 221 | GVILCGPPGTGKTLLAKAVANQTSATFLRVVGSELIQKYLGDGPKLGRRLFRVAEERAPS | 280 |
| | | \|++\| \|\|\|\|\|\|\|\|\|\|\|\|\|\|\| + \|+ + \|\|\|\|+ \|\|+\|+ \|\| \| \|\| +\| + \|\| | |
| 00004 | 1 | GILLYGPPGTGKTLLAKAVAKELGVPFIEISGSELLSKYVGESEKLVRALFSLARKSAPC | 60 |
| NOV12 | 281 | IVFIDEIDAIGTKRYDSNSGGEREIQRTTLELLNQLDGFDSRVDVKAIMATNQIETLDPA | 340 |
| | | \|+\|\|\|\|\|\|+ \|\| \| +\| \| +\|\| ++\|\|\|+ +\| \| \|\|\|+ + \|\|\|\| | |
| 00004 | 61 | IIFIDEIDALAPKRGDVGTGDVSS--RVVNQLLTENMDFEKLSNVIVIGATNRPDLLDPA | 118 |
| NOV12 | 341 | LIRPGRIGRKIEFPLPDEKTKKPIFQIHTSRMTLADDVTLHDSIMAKDDLSGADIKAVCT | 400 |
| | | \|+\|\|\|\| \|+\|\| \|\|\|\|\|+ + \| +\|\| + \| \|\| \| ++ \|\|\|\|+ \|+\| | |
| 00004 | 119 | LLRPGRFDRRIEVPLPDEEERLEILKIHLKKKPLEKDVDLDEIARRTPGFSGADLAALCR | 178 |
| NOV12 | 401 | EAGLMALR 408 | |
| | | \|\| \| \|+\| | |
| 00004 | 179 | EAALRAIR 186 | |

TABLE 12F
Domain Analysis of NOV12

gnl|Smart smart00382, AAA, ATPases associated with a variety of
cellular activities; AAA—ATPases associated with a variety of
cellular activities. This profile/alignment only detects a fraction of
this vast family. The poorly conserved N-terminal helix is missing
from the alignment. (SEQ ID NO:148)
Length 151 residues, 100.0% aligned
Score=62.4 bits (150), Expect=5e-11

| | | | |
|---|---|---|---|
| NOV12 | 218 | PPKGVILCGPPGTGKTLLAKAVANQTSATFLRVV-------------------GSELIQK | 258 |
| | | \| + \|++ \|\|\|\|+\|\|\| \|\|+\|+\| + \|+ \| | |
| 003821 | | PGEVVLIVGPPGSGKTTLARALARELGPDGGGVIYIDGEDLREEALLQLLRLLVLVGEDK | 60 |
| NOV12 | 259 | YLGDGPKLGRELFRVAEERAPSIVFIDEIDAIGTKRYDSNSGGEREIQRTTLELLNQLDG | 318 |
| | | \| \| + \| +\| + \| ++ +\|\|\| ++ + + \|\|\| \| | |
| 00382 | 81 | LSGSGGQRIRLALALARKLKPDVLILDEITSLLDAE-------QEALLLLLEELLRLLLL | 113 |
| NOV12 | 319 | FDSRVDVKAIMATNQIETLDPALIRPGRIGRKIEFPLPD 357 | |
| | | +\| \| \|\| \| \|\|\|+\| \| \|+\| | |
| 00382 | 114 | LLKEENVTVIATTNDETDLIPALLRR-RFDRRIVLLRIL 151 | |

In eukaryotic cells, the vast majority of proteins in the cytosol and nucleus are degraded via the proteosome-ubiquitin pathway. The 26S proteosome is a huge protein degradation machine of 2.5 MDa, built of approximately 35 different subunits. It contains a proteolytic core complex, the 20S proteosome and one or two 19S regulatory complexes which associate with the termini of the barrel-shaped 20S core. The 19S regulatory complex serves to recognize ubiquitylated target proteins and is implicated to have a role in their unfolding and translocation into the interior of the 20S complex where they are degraded into oligopeptides. While much progress has been made in recent years in elucidating the structure, assembly and enzymatic mechanism of the 20S complex, our knowledge of the functional organization of the 19S regulator is rather limited. Most of its subunits have been identified, but specific functions can be assigned to only a few of them. (10582236)

The ATP/ubiquitin-dependent 26S proteosome is a central regulator of cell cycle progression and stress responses. While investigating the application of peptide aldehyde proteasome inhibitors to block signal-induced IkappaBalpha degradation in human LNCaP prostate carcinoma cells, we observed that persistent inhibition of proteosomal activity signals a potent cell death program. Biochemically, this program included substantial upregulation of PAR-4 (prostate apoptosis response-4), a putative pro-apoptotic effector protein and stabilization of c-jun protein, a potent pro-death effector in certain cells. Also observed was modest downregulation of bcl-XL, a pro-survival effector protein. However, in contrast to some recent reports stable, high level, expression of functional bcl-2 protein in prostate carcinoma cells failed to signal protection against cell death induction by proteasome inhibitors. Also in disagreement to a recent report, no evidence was found for activation of the JNK stress kinase pathway. A role for p53, a protein regulated by the proteasome pathway, was ruled out, since comparable cell death induction by proteasome inhibitors occurred in PC-3 cells that do not express functional p53 protein. These data signify that the ubiquitin/proteasome pathway represents a potential therapeutic target for prostate cancers irrespective of bcl-2 expression or p53 mutations (9879995)

The protein similarity information, expression pattern, and map location for NOV12 suggest that NOV12 may have important structural and/or physiological functions characteristic of the 26S protease regulatory subunit family. Therefore, the NOV12 nucleic acids and proteins of the invention are useful in potential therapeutic applications implicated in various diseases and disorders described below and/or other pathologies. For example, the NOV12 compositions of the present invention will have efficacy for treatment of patients suffering from eye/lens disorders including but not limited to cataract and Aphakia, Alzheimer's disease, neurodegenerative disorders, inflammation and modulation of the immune response, viral pathogenesis, aging-related disorders, neurologic disorders, cancer and/or other pathologies and disorders. The NOV12 nucleic acid encoding 26S protease regulatory subunit-like protein, and the 26S protease regulatory subunit-like protein of the invention, or fragments thereof, may further be useful in diagnostic applications, wherein the presence or amount of the nucleic acid or the protein are to be assessed.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199, or a complement of this aforementioned nucleotide sequence, can be isolated using standard molecular biology techniques and the sequence information provided herein. Using all or a portion of the nucleic acid sequence of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199 as a hybridization probe, NOVX molecules can be isolated using standard hybridization and cloning techniques (e.g. as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199, or a portion of this nucleotide sequence (e.g., a fragment that can be used as a probe or primer or a fragment encoding a biologically-active portion of an NOVX polypeptide). A nucleic acid molecule that is complementary to the nucleotide sequence shown NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199 is one that is sufficiently complementary to the nucleotide sequence shown NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199 that it can hydrogen bond with little or no mismatches to the nucleotide sequence shown SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199, as well as a polypeptide possessing NOVX biological activity. Various biological activities of the NOVX proteins are described below.

An NOVX polypeptide is encoded by the open reading frame (“ORF”) of an NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199; or an anti-sense strand nucleotide sequence of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199; or of a naturally occurring mutant of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199.

Probes based on the human NOVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an NOVX protein, such as by measuring a level of an NOVX-encoding nucleic acid in a sample of cells from a subject e.g., detecting NOVX mRNA levels or determining whether a genomic NOVX gene has been mutated or deleted.

“A polypeptide having a biologically-active portion of an NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199, that encodes a polypeptide having an NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences shown in SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199 due to degeneracy of the genetic code and thus encode the same NOVX proteins as that encoded by the nucleotide sequences shown in SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199. In another embodiment, an isolated nucleic acid molecule of the invention has a nucleotide sequence encoding a protein having an amino acid sequence shown in SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64 and 200.

In addition to the human NOVX nucleotide sequences shown in SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding an NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from the human SEQ ID NOS: b 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199 are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199, or fragments, analogs or derivatives thereof, under conditions of moderate stringency is provided. A non-limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199, thereby leading to changes in the amino acid sequences of the encoded NOVX proteins, without altering the functional ability of said NOVX proteins. For example, nucleotide substitutions leading to amino acid substitutions at “non-essential” amino acid residues can be made in the sequence SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64 and 200. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199 yet retain biological activity. In one embodiment, the isolated nucleic acid molecule comprises a nucleotide sequence encoding a protein, wherein the protein comprises an amino acid sequence at least about 45% homologous to the amino acid sequences SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64 and 200. Preferably, the protein encoded by the nucleic acid molecule is at least about 60% homologous to SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64 and 200; more preferably at least about 70% homologous SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64 and 200; still more preferably at least about 80% homologous to SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64 and 200; even more preferably at least about 90% homologous to SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64 and 200; and most preferably at least about 95% homologous to SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64 and 200.

An isolated nucleic acid molecule encoding an NOVX protein homologous to the protein of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64 and 200 can be created by introducing one or more nucleotide substitutions, additions or deletions into the nucleotide sequence of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199, such that one or more amino acid substitutions, additions or deletions are introduced into the encoded protein.

Mutations can be introduced into SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of an NOVX coding sequence, such as by saturation mutagenesis, and the resultant mutants can be screened for NOVX biological activity to identify mutants that retain activity. Following mutagenesis SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and an NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of an NOVX protein of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64 and 200, or antisense nucleic acids complementary to an NOVX nucleic acid sequence of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199, are additionally provided.

In one embodiment, an antisense nucleic acid molecule is antisense to a “coding region” of the coding strand of a nucleotide sequence encoding an NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g. Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for an NOVX-encoding nucleic acid can be designed based upon the nucleotide sequence of an NOVX cDNA disclosed herein (i.e., SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199). For example, a derivative of a Tetrahymena L-19 IVS RNA can be constructed in which the nucleotide sequence of the active site is complementary to the nucleotide sequence to be cleaved in an NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996, supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64 and 200. The invention also includes a mutant or variant protein any of whose residues may be changed from the corresponding residues shown in SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64 and 200 while still encoding a protein that maintains its NOVX activities and physiological functions, or a functional fragment thereof.

In general, an NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, an NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence shown in SEQ ID NOS:2, 4, 6, 8, 10, 12, 14, 16, 18, 20, 22, 24, 26, 28,30, 32, 34, 36, 38, 40, 42, 44, 46, 48, 50, 52, 54, 56, 58, 60, 62, 64 and 200) that include fewer amino acids than the full-length NOVX proteins, and exhibit at least one activity of an NOVX protein. Typically, biologically-active portions comprise a domain or motif with at least one activity of the NOVX protein. A biologically-active portion of an NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence shown SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64 and 200. In other embodiments, the NOVX protein is substantially homologous to SEQ ID NOS:2, 4,6, 8, 10, 12, 14, 16, 18, 20, 22, 24, 26, 28, 30, 32, 34, 36, 38, 40, 42, 44, 46, 48, 50, 52, 54, 56, 58, 60, 62, 64 and 200, and retains the functional activity of the protein of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64 and 200, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64 and 200, and retains the functional activity of the NOVX proteins of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64 and 200.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, an NOVX “chimeric protein” or “fusion protein” comprises an NOVX polypeptide operatively-linked to a non-NOVX polypeptide. An “NOVX polypeptide” refers to a polypeptide having an amino acid sequence corresponding to an NOVX protein SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64 and 200, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within an NOVX fusion protein the NOVX polypeptide can correspond to all or a portion of an NOVX protein. In one embodiment, an NOVX fusion protein comprises at least one biologically-active portion of an NOVX protein. In another embodiment, an NOVX fusion protein comprises at least two biologically-active portions of an NOVX protein. In yet another embodiment, an NOVX fusion protein comprises at least three biologically-active portions of an NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is an NOVX protein containing a heterologous signal sequence at its N-terminus. In certain host cells (e.g. mammalian host cells), expression and/or secretion of NOVX can be increased through use of a heterologous signal sequence.

In yet another embodiment, the fusion protein is an NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between an NOVX ligand and an NOVX protein on the surface of a cell, to thereby suppress NOVX-mediated signal transduction in vivo. The NOVX-immunoglobulin fusion proteins can be used to affect the bioavailability of an NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with an NOVX ligand.

An NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of an NOVX protein. In one embodiment, a library of coding sequence fragments can be generated by treating a double stranded PCR fragment of an NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

Also included in the invention are antibodies to NOVX proteins, or fragments of NOVX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab}, and F_{(ab′)2} fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated NOVX-related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX-related protein that is located on the surface of the protein, e.g., a hydrophilic region. A hydrophobicity analysis of the human NOVX-related protein sequence will indicate which regions of a NOVX-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow and Lane, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, MONOCLONAL ANTIBODIES: PRINCIPLES AND PRACTICE, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., MONOCLONAL ANTIBODY PRODUCTION TECHNIQUES AND APPLICATIONS, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al,(Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

Fab Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5): 1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Anti-NOVX antibodies may be used in methods known within the art relating to the localization and/or quantitation of an NOVX protein (e.g., for use in measuring levels of the NOVX protein within appropriate physiological samples, for use in diagnostic methods, for use in imaging the protein, and the like). In a given embodiment, antibodies for NOVX proteins, or derivatives, fragments, analogs or homologs thereof, that contain the antibody derived binding domain, are utilized as pharmacologically-active compounds (hereinafter “Therapeutics”).

An anti-NOVX antibody (e.g., monoclonal antibody) can be used to isolate an NOVX polypeptide by standard techniques, such as affinity chromatography or immunoprecipitation. An anti-NOVX antibody can facilitate the purification of natural NOVX polypeptide from cells and of recombinantly-produced NOVX polypeptide expressed in host cells. Moreover, an anti-NOVX antibody can be used to detect NOVX protein (e.g., in a cellular lysate or cell supernatant) in order to evaluate the abundance and pattern of expression of the NOVX protein. Anti-NOVX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, □-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

NOVX Recombinant Expression Vectors and Host Cells

Another aspect of the invention pertains to vectors, preferably expression vectors, containing a nucleic acid encoding an NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264, 166). Developmentally-regulated promoters are also encompassed, e.g., the murine hox promoters (Kessel and Gruss, 1990. Science 249: 374-379) and the □-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199 can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of an NOVX gene into which a deletion, addition or substitution has been introduced to thereby alter, e.g., functionally disrupt, the NOVX gene. The NOVX gene can be a human gene (e.g., the cDNA of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199), but more preferably, is a non-human homologue of a human NOVX gene. For example, a mouse homologue of human NOVX gene of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199 can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in an NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of an NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to an NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with an NOVX target molecule. As used herein, a “target molecule” is a molecule with which an NOVX protein binds or interacts in nature, for example, a molecule on the surface of a cell which expresses an NOVX interacting protein, a molecule on the surface of a second cell, a molecule in the extracellular milieu, a molecule associated with the internal surface of a cell membrane or a cytoplasmic molecule. An NOVX target molecule can be a non-NOVX molecule or an NOVX protein or polypeptide of the invention. In one embodiment, an NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with an NOVX target molecule can be accomplished by one of the methods described above for determining direct binding. In one embodiment, determining the ability of the NOVX protein to bind to or interact with an NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to an NOVX target molecule by one of the methods described above for determining direct binding. In an alternative embodiment, determining the ability of the test compound to modulate the activity of NOVX protein can be accomplished by determining the ability of the NOVX protein further modulate an NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with an NOVX protein, wherein determining the ability of the test compound to interact with an NOVX protein comprises determining the ability of the NOVX protein to preferentially bind to or modulate the activity of an NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl--N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences, SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in an NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”).

Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in an NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an NOVX-protein, or the misexpression of the NOVX gene. For example, such genetic lesions can be detected by ascertaining the existence of at least one of: (i) a deletion of one or more nucleotides from an NOVX gene; (ii) an addition of one or more nucleotides to an NOVX gene; (iii) a substitution of one or more nucleotides of an NOVX gene, (iv) a chromosomal rearrangement of an NOVX gene; (v) an alteration in the level of a messenger RNA transcript of an NOVX gene, (vi) aberrant modification of an NOVX gene, such as of the methylation pattern of the genomic DNA, (vii) the presence of a non-wild-type splicing pattern of a messenger RNA transcript of an NOVX gene, (viii) a non-wild-type level of an NOVX protein, (ix) allelic loss of an NOVX gene, and (x) inappropriate post-translational modification of an NOVX protein. As described herein, there are a large number of assay techniques known in the art which can be used for detecting lesions in an NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared.

Differences in fragment length sizes between sample and control DNA indicates mutations in the sample DNA. Moreover, the use of sequence specific ribozymes (see, e.g. U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g. Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g. Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharnacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C 19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof, or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, an NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of an NOVX protein, a peptide, an NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering an NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example 1

### Identification of NOVX Nucleic Acids

ThlastN using CuraGen Corporation's sequence file for polypeptides or homologs was run against the Genomic Daily Files made available by GenBank or from files downloaded from the individual sequencing centers. Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The novel NOVX target sequences identified in the present invention were subjected to the exon linking process to confirm the sequence. PCR primers were designed by starting at the most upstream sequence available, for the forward primer, and at the most downstream sequence available for the reverse primer. Table 11A shows the sequences of the PCR primers used for obtaining different clones. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. Table 17B shows a list of these bacterial clones. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

Physical clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

### Example 2

### Identification of Single Nucleotide Polymorphisms in NOVX Nucleic Acid Sequences

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

SeqCalling assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraTools™ program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the full length sequence.

### Example 3

### Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on a Perkin-Elmer Biosystems ABI PRISM® 7700 Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), AI_comprehensive_panel (containing normal tissue and samples from autoinflammatory diseases), Panel CNSD.01 (containing samples from normal and diseased brains) and CNS_neurodegeneration_panel (containing samples from normal and diseased brains).

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (PE Biosystems; Catalog No. 4309169) and gene-specific primers according to the manufacturer's instructions. Probes and primers were designed for each assay according to Perkin Elmer Biosystem's Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tₘ) range=58°-60° C., primer optimal Tm=59° C., maximum primer difference 2° C., probe does not have 5′ G, probe Tₘ must be 10° C. greater than primer Tₘ, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: Normalized RNA from each tissue and each cell line was spotted in each well of a 96 well PCR plate (Perkin Elmer Biosystems). PCR cocktails including two probes (a probe specific for the target clone and another gene-specific probe multiplexed with the target probe) were set up using 1×TaqMan™ PCR Master Mix for the PE Biosystems 7700, with 5 mM MgCl2, dNTPs (dA, G, C, U at 1:1:1:2 ratios), 0.25 U/ml AmpliTaq Gold™ (PE Biosystems), and 0.4 U/μl RNase inhibitor, and 0.25 U/μl reverse transcriptase. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:

ca.=carcinoma,

*=established from metastasis,

met=metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

General Screening Panel v1.4

The plates for Panel 1.4 include 2 control wells (genomic DNA control and chemistry control) and 94 wells containing cDNA from various samples. The samples in Panel 1.4 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panel 1.4 are widely available through the American Type Culture Collection (ATCC), a repository for cultured cell lines, and were cultured using the conditions recommended by the ATCC. The normal tissues found on Panel 1.4 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

Panels 2D and 2.2

The plates for Panels 2D and 2.2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologists at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

Panel 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

Panels 4D, 4R, and 4.1D

Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) were employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵ M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵ M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah, 100 μM non essential amino acids (Gibco), 1 mM sodium pyruvate (Gibco), mercaptoethanol 5.5×10⁻⁵ M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 gM non essential amino acids (Gibco), 1 mM sodium pyruvate (Gibco), mercaptoethanol 5.5×10⁻⁵ M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. Then CD45RO beads were used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24, 48 and 72 hours.

To prepare the primary and secondary Th1/Th2 and Tr1 cells, six-well Falcon plates were coated overnight with 10 μg/ml anti-CD28 (Pharmingen) and 2 tg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (4 ng/ml). IL-12 (5 ng/ml) and anti-IL4 (1 □g/ml) were used to direct to Th1, while IL-4 (5 ng/ml) and anti-IFN gamma (1 □g/ml) were used to direct to Th2 and IL-10 at 5 ng/ml was used to direct to Trl. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 □g/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20 degrees C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37 degrees C. for 30 minutes to remove contaminating genomic DNA, extracted once with phenol chloroform and re-precipitated with 1/10 volume of 3 M sodium acetate and 2 volumes of 100% ethanol. The RNA was spun down and placed in RNAse free water. RNA was stored at −80 degrees C.

Panels CNSD.01, CNS_{—}1 and CNS_{—}1.1

The plates for Panel CNSD.01, CNS_{—}1 and CNS1.1 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) pateins, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Broddmann Area 21), parietal cortex (Broddmann area 7), and occipital cortex (Brodmann area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V1.0 panel, the following abbreviations are used:

AD=Alzheimer's disease brain; patient was demented and showed AD-like pathology upon autopsy

Control=Control brains; patient not demented, showing no neuropathology

Control (Path)=Control brains; pateint not demented but showing sever AD-like pathology

SupTemporal Ctx=Superior Temporal Cortex

Inf Temporal Ctx=Inferior Temporal Cortex

NOV1: ALPHA-2-MACROGLOBULIN

Expression of the NOV1 gene (SC_{—}78316254_A) was assessed using the primer-probe sets Ag1180 and Ag1312, described in Table 13. Results from RTQ-PCR runs are shown in Tables 14, 15, 16, 17, 18 and 19.

TABLE 13

Probe Name Ag1180/Ag1312 (Identical Sequence)

| | | | | | |
|---|---|---|---|---|---|
| | | | | Start | SEQ ID |
| Primers | Sequences | TM | Length | Position | NO: |

| | | | | | |
|---|---|---|---|---|---|
| Forward | 5′-CCTGGAAATAGGGTACCAGAAG-3′ | 59 | 22 | 3027 | 149 |
| Probe | FAM-5′ACACAGCAATGGCTCATACAGTGCCT- | 68.9 | 26 | 3063 | 150 |
| | 3′-TAMRA | | | | |
| Reverse | 5′-TCAGCCATGTGTTTCCATTT-3′ | 59 | 20 | 3105 | 151 |

TABLE 14
Panel 1.2

| | Relative | Relative |
|---|---|---|
| | Expression (%) | Expression (%) |
| | 1.2tm1392f_{—} | 1.2tm1998f_{—} |
| Tissue Name | ag1180 | ag1180 |
| Endothelial cells | 0.0 | 0.0 |
| Heart (fetal) | 0.0 | 0.0 |
| Pancreas | 0.0 | 0.0 |
| Pancreatic ca. CAPAN 2 | 1.0 | 2.6 |
| Adrenal Gland (new lot*) | 0.0 | 0.0 |
| Thyroid | 0.0 | 0.0 |
| Salivary gland | 1.7 | 7.6 |
| Pituitary gland | 0.2 | 0.0 |
| Brain (fetal) | 0.1 | 0.0 |
| Brain (whole) | 0.6 | 0.4 |
| Brain (amygdala) | 0.8 | 1.1 |
| Brain (cerebellum) | 0.0 | 0.1 |
| Brain (hippocampus) | 1.1 | 2.6 |
| Brain (thalamus) | 0.3 | 1.4 |
| Cerebral Cortex | 2.3 | 4.7 |
| Spinal cord | 3.0 | 1.2 |
| CNS ca. (glio/astro) U87-MG | 0.0 | 0.0 |
| CNS ca. (glio/astro) U-118-MG | 0.0 | 0.0 |
| CNS ca. (astro) SW1783 | 0.0 | 0.0 |
| CNS ca.* (neuro; met) SK-N-AS | 0.0 | 0.0 |
| CNS ca. (astro) SF-539 | 0.0 | 0.0 |
| CNS ca. (astro) SNB-75 | 0.0 | 0.0 |
| CNS ca. (glio) SNB-19 | 0.0 | 0.0 |
| CNS ca. (glio) U251 | 0.0 | 0.0 |
| CNS ca. (glio) SF-295 | 0.0 | 0.0 |
| Heart | 0.0 | 0.2 |
| Skeletal Muscle (new lot*) | 5.4 | 0.0 |
| Bone marrow | 0.0 | 0.0 |
| Thymus | 0.5 | 0.5 |
| Spleen | 0.0 | 0.0 |
| Lymph node | 0.0 | 0.0 |
| Colorectal | 0.0 | 0.0 |
| Stomach | 4.5 | 2.1 |
| Small intestine | 0.0 | 0.0 |
| Colon ca. SW480 | 0.0 | 0.0 |
| Colon ca.* (SW480 met) SW620 | 0.0 | 0.0 |
| Colon ca. HT29 | 0.0 | 0.0 |
| Colon ca. HCT-116 | 0.0 | 0.0 |
| Colon ca. CaCo-2 | 0.0 | 0.0 |
| 83219 CC Well to Mod Diff | 0.0 | 0.0 |
| (ODO3866) | | |
| Colon ca. HCC-2998 | 0.0 | 0.0 |
| Gastric ca.* (liver met) NCI-N87 | 100.0 | 100.0 |
| Bladder | 0.0 | 0.0 |
| Trachea | 1.1 | 0.2 |
| Kidney | 0.0 | 0.0 |
| Kidney (fetal) | 0.0 | 0.0 |
| Renal ca. 786-0 | 0.0 | 0.0 |
| Renal ca. A498 | 0.0 | 0.0 |
| Renal ca. RXF 393 | 0.0 | 0.0 |
| Renal ca. ACHN | 0.0 | 0.0 |
| Renal ca. UO-31 | 0.0 | 0.0 |
| Renal ca. TK-10 | 0.0 | 0.0 |
| Liver | 0.0 | 0.0 |
| Liver (fetal) | 0.0 | 0.0 |
| Liver ca. (hepatoblast) HepG2 | 0.0 | 0.0 |
| Lung | 0.0 | 0.0 |
| Lung (fetal) | 0.0 | 0.0 |
| Lung ca. (small cell) LX-1 | 0.0 | 0.0 |
| Lung ca. (small cell) NCI-H69 | 0.0 | 0.0 |
| Lung ca. (s.cell var.) SHP-77 | 0.0 | 0.0 |
| Lung ca. (large cell) NCI-H460 | 0.0 | 0.0 |
| Lung ca. (non-sm.cell) A549 | 0.0 | 0.0 |
| Lung ca. (non-s.cell) NCI-H23 | 0.0 | 0.0 |
| Lung ca (non-s.cell) HOP-62 | 0.0 | 0.0 |
| Lung ca. (non-s.cl) NCI-H522 | 0.0 | 0.1 |
| Lung ca. (squam.) SW 900 | 0.0 | 0.0 |
| Lung ca. (squam.) NCI-H596 | 0.0 | 0.0 |
| Mammary gland | 1.1 | 1.8 |
| Breast ca.* (pl. effusion) MCF-7 | 0.0 | 0.0 |
| Breast ca.* (pl.ef) MDA-MB-231 | 0.0 | 0.0 |
| Breast ca.* (pl. effusion) T47D | 0.0 | 0.0 |
| Breast ca. BT-549 | 0.0 | 0.0 |
| Breast ca. MDA-N | 0.0 | 0.0 |
| Ovary | 0.1 | 0.3 |
| Ovarian ca. OVCAR-3 | 0.2 | 0.2 |
| Ovarian ca. OVCAR-4 | 0.0 | 0.0 |
| Ovarian ca. OVCAR-5 | 0.0 | 0.0 |
| Ovarian ca. OVCAR-8 | 0.0 | 0.0 |
| Ovarian ca. IGROV-1 | 0.0 | 0.2 |
| Ovarian ca.* (ascites) SK-OV-3 | 0.0 | 0.0 |
| Uterus | 0.0 | 0.2 |
| Placenta | 1.6 | 0.4 |
| Prostate | 0.4 | 1.4 |
| Prostate ca.* (bone met) PC-3 | 0.0 | 0.0 |
| Testis | 4.0 | 0.7 |
| Melanoma Hs688 (A).T | 0.0 | 0.0 |
| Melanoma* (met) Hs688 (B).T | 0.0 | 0.0 |
| Melanoma UACC-62 | 0.0 | 0.0 |
| Melanoma M14 | 0.0 | 0.0 |
| Melanoma LOX IMVI | 0.0 | 0.0 |
| Melanoma* (met) SK-MEL-5 | 0.0 | 0.0 |

TABLE 15
Panel 1.3D

| | Relative | | Relative |
|---|---|---|---|
| | Expression (%) | | Expression (%) |
| | 1.3dx4tm5588 | | 1.3dx4tm5588 |
| Tissue Name | f_ag1180_a2 | Tissue Name | f_ag1180_a2 |
| Liver adenocarcinoma | 0.0 | Kidney (fetal) | 0.0 |
| Pancreas | 0.0 | Renal ca. 786-0 | 0.0 |
| Pancreatic ca. CAPAN 2 | 11.3 | Renal ca. A498 | 0.0 |
| Adrenal gland | 0.0 | Renal ca. RXF 393 | 0.0 |
| Thyroid | 1.6 | Renal ca. ACHN | 0.0 |
| Salivary gland | 7.6 | Renal ca. UO-31 | 0.0 |
| Pituitary gland | 0.8 | Renal ca. TK-10 | 0.0 |
| Brain (fetal) | 4.5 | Liver | 0.0 |
| Brain (whole) | 8.9 | Liver (fetal) | 0.0 |
| Brain (amygdala) | 22.7 | Liver ca. (hepatoblast) HepG2 | 0.0 |
| Brain (cerebellum) | 8 | Lung | 0.0 |
| Brain (hippocampus) | 4.9 | Lung (fetal) | 0.9 |
| Brain (substantia nigra) | 1.9 | Lung ca. (small cell) LX-1 | 0.0 |
| Brain (thalamus) | 6.7 | Lung ca. (small cell) NCI-H69 | 0.0 |
| Cerebral Cortex | 6.8 | Lung ca. (s.cell var.) SHP-77 | 0.0 |
| Spinal cord | 47.6 | Lung ca. (large cell) NCI-H460 | 0.0 |
| CNS ca. (glio/astro) U87-MG | 0.0 | Lung ca. (non-sm. cell) A549 | 0.0 |
| CNS ca. (glio/astro) U-118-MG | 0.0 | Lung ca. (non-s.cell) NCI-H23 | 0.0 |
| CNS ca. (astro) SW1783 | 0.0 | Lung ca (non-s.cell) HOP-62 | 0.0 |
| CNS ca.* (neuro; met) SK-N-AS | 0.0 | Lung ca. (non-s.cl) NCI-H522 | 0.0 |
| CNS ca. (astro) SF-539 | 0.0 | Lung ca. (squam.) SW 900 | 0.0 |
| CNS ca. (astro) SNB-75 | 0.0 | Lung ca. (squam.) NCI-H596 | 0.0 |
| CNS ca. (glio) SNB-19 | 0.0 | Mammary gland | 3.0 |
| CNS ca. (glio) U251 | 0.0 | Breast ca.* (pl. effusion) MCF-7 | 0.9 |
| CNS ca. (glio) SF-295 | 0.0 | Breast ca.* (pl.ef) | 0.0 |
| | | MDA-MB-231 | |
| Heart (fetal) | 0.0 | Breast ca.* (pl. effusion) T47D | 0.0 |
| Heart | 0.0 | Breast ca. BT-549 | 0.0 |
| Fetal Skeletal | 0.0 | Breast ca. MDA-N | 0.0 |
| Skeletal muscle | 0.9 | Ovary | 2.5 |
| Bone marrow | 0.0 | Ovarian ca. OVCAR-3 | 0.2 |
| Thymus | 14 | Ovarian ca. OVCAR-4 | 0.0 |
| Spleen | 1.4 | Ovarian ca. OVCAR-5 | 0.0 |
| Lymph node | 0.0 | Ovarian ca. OVCAR-8 | 0.0 |
| Colorectal | 0.0 | Ovarian ca. IGROV-1 | 0.0 |
| Stomach | 17.8 | Ovarian ca.* (ascites) SK-OV-3 | 0.3 |
| Small intestine | 0.0 | Uterus | 0.0 |
| Colon ca. SW480 | 0.0 | Placenta | 11.3 |
| Colon ca.* (SW480 met) | 0.0 | Prostate | 0.0 |
| SW620.0 | | | |
| Colon ca. HT29 | 0.0 | Prostate ca.* (bone met) PC-3 | 0.0 |
| Colon ca. HCT-116 | 0.0 | Testis | 17.1 |
| Colon ca. CaCo-2 | 0.0 | Melanoma Hs688 (A).T | 0.0 |
| 83219 CC Well to Mod Diff | 0.0 | Melanoma* (met) Hs688 (B).T | 0.0 |
| (ODO3866) | | | |
| Colon ca. HCC-2998 | 0.0 | Melanoma UACC-62 | 0.9 |
| Gastric ca.* (liver met) | 100 | Melanoma M14 | 0.0 |
| NCI-N87 | | | |
| Bladder | 0.0 | Melanoma LOX IMVI | 0.0 |
| Trachea | 7.6 | Melanoma* (met) SK-MEL-5 | 0.0 |
| Kidney | 0.4 | Adipose | 0.0 |

TABLE 16
Panel 2D

| | Relative | | Relative |
|---|---|---|---|
| | Expression (%) | | Expression (%) |
| | 2dx4tm4715f_{—} | | 2dx4tm4715f_{—} |
| Tissue Name | ag1180_a2 | Tissue Name | ag1180_a2 |
| Normal Colon GENPAK 061003 | 0.1 | Kidney NAT Clontech 8120608 | 0.0 |
| 83219 CC Well to Mod Diff | 0.1 | Kidney Cancer Clontech 8120613 | 0.0 |
| (ODO3866) | | | |
| 83220 CC NAT (ODO3866) | 0.0 | Kidney NAT Clontech 8120614 | 0.0 |
| 83221 CC Gr.2 rectosigmoid | 0.0 | Kidney Cancer Clontech 9010320 | 0.0 |
| (ODO3868) | | | |
| 83222 CC NAT (ODO3868) | 0.0 | Kidney NAT Clontech 9010321 | 0.0 |
| 83235 CC Mod Diff (ODO3920) | 0.0 | Normal Uterus GENPAK 061018 | 0.0 |
| 83236 CC NAT (ODO3920) | 0.0 | Uterus Cancer GENPAK 064011 | 0.4 |
| 83237 CC Gr.2 ascend colon | 0.0 | Normal Thyroid Clontech A + | 0.4 |
| (ODO3921) | | 6570-1 | |
| 83238 CC NAT (ODO3921) | 0.0 | Thyroid Cancer GENPAK 064010 | 0.0 |
| 83241 CC from Partial | 0.0 | Thyroid Cancer INVITROGEN | 0.0 |
| Hepatectomy (ODO4309) | | A302152 | |
| 83242 Liver NAT (ODO4309) | 0.0 | Thyroid NAT INVITROGEN | 0.0 |
| | | A302153 | |
| 87472 Colon mets to lung | 0.0 | Normal Breast GENPAK 061019 | 0.1 |
| (OD04451-01) | | | |
| 87473 Lung NAT (OD04451-02) | 0.0 | 84877 Breast Cancer (OD04566) | 0.0 |
| Normal Prostate Clontech A + | 3.3 | 85975 Breast Cancer | 0.0 |
| 6546-1 | | (OD04590-01) | |
| 84140 Prostate Cancer (OD04410) | 0.0 | 85976 Breast Cancer Mets | 0.0 |
| | | (OD04590-03) | |
| 84141 Prostate NAT (OD04410) | 0.6 | 87070 Breast Cancer Metastasis | 0.0 |
| | | (OD04655-05) | 0.0 |
| 87073 Prostate Cancer | 0.5 | GENPAK Breast Cancer 064006 | 1.2 |
| (OD04720-01) | | | |
| 87074 Prostate NAT | 0.8 | Breast Cancer Res. Gen. 1024 | 0.0 |
| (OD04720-02) | | | |
| Normal Lung GENPAK 061010 | 0.1 | Breast Cancer Clontech 9100266 | 0.0 |
| 83239 Lung Met to Muscle | 0.0 | Breast NAT Clontech 9100265 | 0.0 |
| (ODO4286) | | | |
| 83240 Muscle NAT (ODO4286) | 0.0 | Breast Cancer INVITROGEN | 0.2 |
| | | A209073 | |
| 84136 Lung Malignant Cancer | 0.0 | Breast NAT INVITROGEN | 0.1 |
| (OD03126) | | A2090734 | |
| 84137 Lung NAT (OD03126) | 0.0 | Normal Liver GENPAK 061009 | 0.0 |
| 84871 Lung Cancer (OD04404) | 18.5 | Liver Cancer GENPAK 064003 | 0.0 |
| 84872 Lung NAT (OD04404) | 0.0 | Liver Cancer Research Genetics | 0.0 |
| | | RNA 1025 | |
| 84875 Lung Cancer (OD04565) | 0.1 | Liver Cancer Research Genetics | 0.0 |
| | | RNA 1026 | |
| 84876 Lung NAT (OD04565) | 0.0 | Paired Liver Cancer Tissue | 0.0 |
| | | Research Genetics RNA 6004-T | |
| 85950 Lung Cancer (OD04237-01) | 0.0 | Paired Liver Tissue Research | 0.0 |
| | | Genetics RNA 6004-N | |
| 85970 Lung NAT (OD04237-02) | 0.0 | Paired Liver Cancer Tissue | 0.0 |
| | | Research Genetics RNA 6005-T | |
| 83255 Ocular Mel Met to Liver | 0.1 | Paired Liver Tissue Research | 0.0 |
| (ODO4310) | | Genetics RNA 6005-N | |
| 83256 Liver NAT (ODO4310) | 0.0 | Normal Bladder GENPAK 061001 | 0.0 |
| 84139 Melanoma Mets to Lung | 0.0 | Bladder Cancer Research Genetics | 0.0 |
| (OD04321) | | RNA 1023 | |
| 84138 Lung NAT(OD04321) | 0.0 | Bladder Cancer INVITROGEN | 13.0 |
| | | A302173 | |
| Normal Kidney GENPAK 061008 | 0.0 | 87071 Bladder Cancer | 0.6 |
| | | (OD04718-01) | |
| 83786 Kidney Ca, Nuclear grade 2 | 0.0 | 87072 Bladder Normal Adjacent | 0.0 |
| (OD04338) | | (OD04718-03) | |
| 83787 Kidney NAT (OD04338) | 0.0 | Normal Ovary Res. Gen. | 0.0 |
| 83788 Kidney Ca Nuclear grade | 0.0 | Ovarian Cancer GENPAK 064008 | 0.8 |
| 1/2 (OD04339) | | | |
| 83789 Kidney NAT (OD04339) | 0.0 | 87492 Ovary Cancer | 100 |
| | | (OD04768-07) | |
| 83790 Kidney Ca, Clear cell type | 0.0 | 87493 Ovary NAT (OD04768-08) | 0.0 |
| (OD04340) | | | |
| 83791 Kidney NAT (OD04340) | 0.0 | Normal Stomach GENPAK | 0.0 |
| | | 061017 | |
| 83792 Kidney Ca, Nuclear grade 3 | 0.0 | Gastric Cancer Clontech 9060358 | 0.0 |
| (OD04348) | | | |
| 83793 Kidney NAT (OD04348) | 0.0 | NAT Stomach Clontech 9060359 | 0.0 |
| 87474 Kidney Cancer | 0.0 | Gastric Cancer Clontech 9060395 | 0.2 |
| (OD04622-01) | | | |
| 87475 Kidney NAT (OD04622-03) | 0.0 | NAT Stomach Clontech 9060394 | 0.0 |
| 85973 Kidney Cancer | 0.0 | Gastric Cancer Clontech 9060397 | 0.1 |
| (OD04450-01) | | | |
| 85974 Kidney NAT (OD04450-03) | 0.0 | NAT Stomach Clontech 9060396 | 0.0 |
| Kidney Cancer Clontech 8120607 | 0.0 | Gastric Cancer GENPAK 064005 | 0.0 |

TABLE 17
Panel 2.2

| | Relative | | Relative |
|---|---|---|---|
| | Expression (%) | | Expression (%) |
| | 2.2x4tm6329f_{—} | | 2.2x4tm6329f_{—} |
| Tissue Name | ag1180_a1 | Tissue Name | ag1180_a1 |
| Normal Colon GENPAK 061003 | 0.0 | 83793 Kidney NAT (OD04348) | 0.0 |
| 97759 Colon cancer (OD06064) | 5.0 | 98938 Kidney malignant cancer | 0.4 |
| | | (OD06204B) | |
| 97760 Colon cancer NAT | 1.6 | 98939 Kidney normal adjacent | 0.0 |
| (OD06064) | | tissue (OD06204E) | |
| 97778 Colon cancer (OD06159) | 0.0 | 85973 Kidney Cancer (OD04450-01) | 0.0 |
| 97779 Colon cancer NAT | 0.0 | 85974 Kidney NAT (OD04450-03) | 1.1 |
| (OD06159) | | | |
| 98861 Colon cancer (OD06297-04) | 0.0 | Kidney Cancer Clontech 8120613 | 0.0 |
| 98862 Colon cancer NAT | 0.0 | Kidney NAT Clontech 8120614 | 0.0 |
| (OD06297-015) | | | |
| 83237 CC Gr.2 ascend colon | 0.0 | Kidney Cancer Clontech 9010320 | 0.0 |
| (ODO3921) | | | |
| 83238 CC NAT (ODO3921) | 0.0 | Kidney NAT Clontech 9010321 | 1.0 |
| 97766 Colon cancer metastasis | 0.0 | Kidney Cancer Clontech 8120607 | 0.0 |
| (OD06104) | | | |
| 97767 Lung NAT (OD06104) | 0.0 | Kidney NAT Clontech 8120608 | 0.0 |
| 87472 Colon mets to lung | 0.0 | Normal Uterus GENPAK 061018 | 3.4 |
| (OD04451-01) | | | |
| 87473 Lung NAT (OD04451-02) | 0.0 | Uterus Cancer GENPAK 064011 | 0.0 |
| Normal Prostate Clontech A + | 0.0 | Normal Thyroid Clontech A + | 0.0 |
| 6546-1 (8090438) | | 6570-1 (7080817) | |
| 84140 Prostate Cancer (OD04410) | 0.0 | Thyroid Cancer GENPAK 064010 | 0.0 |
| 84141 Prostate NAT (OD04410) | 1.3 | Thyroid Cancer INVITROGEN | 0.0 |
| | | A302152 | |
| Normal Ovary Res. Gen. | 1.9 | Thyroid NAT INVITROGEN | 0.0 |
| | | A302153 | |
| 98863 Ovarian cancer | 0.0 | Normal Breast GENPAK 061019 | 0.0 |
| (OD06283-03) | | | |
| 98865 Ovarian cancer | 0.0 | 84877 Breast Cancer (OD04566) | 0.0 |
| NAT/fallopian tube (OD06283-07) | | | |
| Ovarian Cancer GENPAK 064008 | 8.2 | Breast Cancer Res. Gen. 1024 | 0.0 |
| 97773 Ovarian cancer (OD06145) | 0.0 | 85975 Breast Cancer | 0.0 |
| | | (OD04590-01) | |
| 97775 Ovarian cancer NAT | 0.0 | 85976 Breast Cancer Mets | 0.0 |
| (OD06145) | | (OD04590-03) | |
| 98853 Ovarian cancer | 5.5 | 87070 Breast Cancer Metastasis | 0.4 |
| (OD06455-03) | | (OD04655-05) | |
| 98854 Ovarian NAT | 0.0 | GENPAK Breast Cancer 064006 | 12.1 |
| (OD06455-07) Fallopian tube | | | |
| Normal Lung GENPAK 061010 | 0.0 | Breast Cancer Clontech 9100266 | 0.0 |
| 92337 Invasive poor diff. lung | 9.6 | Breast NAT Clontech 9100265 | 0.0 |
| adeno (ODO4945-01 | | | |
| 92338 Lung NAT (ODO4945-03) | 0.0 | Breast Cancer INVITROGEN | 0.3 |
| | | A209073 | |
| 84136 Lung Malignant Cancer | 0.0 | Breast NAT INVITROGEN | 0.0 |
| (OD03126) | | A2090734 | |
| 84137 Lung NAT (OD03126) | 0.7 | 97763 Breast cancer (OD06083) | 1.8 |
| 90372 Lung Cancer (OD05014A) | 0.0 | 97764 Breast cancer node | 0.0 |
| | | metastasis (OD06083) | |
| 90373 Lung NAT (OD05014B) | 0.0 | Normal Liver GENPAK 061009 | 0.2 |
| 97761 Lung cancer (OD06081) | 5.9 | Liver Cancer Research Genetics | 0.0 |
| | | RNA 1026 | |
| 97762 Lung cancer NAT | 0.0 | Liver Cancer Research Genetics | 1.0 |
| (OD06081) | | RNA 1025 | |
| 85950 Lung Cancer (OD04237-01) | 0.0 | Paired Liver Cancer Tissue | 2.1 |
| | | Research Genetics RNA 6004-T | |
| 85970 Lung NAT (OD04237-02) | 0.0 | Paired Liver Tissue Research | 0.0 |
| | | Genetics RNA 6004-N | |
| 83255 Ocular Mel Met to Liver | 0.0 | Paired Liver Cancer Tissue | 0.0 |
| (ODO4310) | | Research Genetics RNA 6005-T | |
| 83256 Liver NAT (ODO4310) | 0.0 | Paired Liver Tissue Research | 0.5 |
| | | Genetics RNA 6005-N | |
| 84139 Melanoma Mets to Lung | 0.0 | Liver Cancer GENPAK 064003 | 0.5 |
| (OD04321) | | | |
| 84138 Lung NAT (OD04321) | 0.0 | Normal Bladder GENPAK 061001 | 0.0 |
| Normal Kidney GENPAK 061008 | 0.0 | Bladder Cancer Research Genetics | 0.0 |
| | | RNA 1023 | |
| 83786 Kidney Ca, Nuclear grade 2 | 0.0 | Bladder Cancer INVITROGEN | 100.0 |
| (OD04338) | | A302173 | |
| 83787 Kidney NAT (OD04338) | 0.0 | Normal Stomach GENPAK | 1.6 |
| | | 061017 | |
| 83788 Kidney Ca Nuclear grade | 0.4 | Gastric Cancer Clontech 9060397 | 0.0 |
| 1/2 (OD04339) | | | |
| 83789 Kidney NAT (OD04339) | 0.0 | NAT Stomach Clontech 9060396 | 0.0 |
| 83790 Kidney Ca, Clear cell type | 0.0 | Gastric Cancer Clontech 9060395 | 2.3 |
| (OD04340) | | | |
| 83791 Kidney NAT (OD04340) | 0.0 | NAT Stomach Clontech 9060394 | 1.9 |
| 83792 Kidney Ca, Nuclear grade 3 | 0.0 | Gastric Cancer GENPAK 064005 | 0.0 |
| (OD04348) | | | |

TABLE 18
Panel 3D

| | Relative | | Relative |
|---|---|---|---|
| | Expression (%) | | Expression (%) |
| | 3dtm4779f_{—} | | 3dtm4779f_{—} |
| Tissue Name | ag1180 | Tissue Name | ag1180 |
| 94905_Daoy_Medulloblastoma/ | 0 | 94954_Ca Ski_Cervical | 0 |
| Cerebellum_sscDNA | | epidermoid carcinoma | |
| | | (metastasis)_sscDNA | |
| 94906_TE671_Medulloblastom/ | 0 | 94955_ES-2_Ovarian clear cell | 0 |
| Cerebellum_sscDNA | | carcinoma_sscDNA | |
| 94907_D283 | 0 | 94957_Ramos/6h stim_Stimulated | 0 |
| Med_Medulloblastoma/ | | with PMA/ionomycin 6h_sscDNA | |
| Cerebellum_sscDNA | | | |
| 94908_PFSK-1_Primitive | 0 | 94958_Ramos/14h stim_{—} | 0 |
| Neuroectodermal/Cerebellum ssc | | Stimulated with PMA/ionomycin | |
| DNA | | 14h_sscDNA | |
| 94909_XF-498_CNS_sscDNA | 0.0 | 94962_MEG-01_Chronic | 0.0 |
| | | myelogenous leukemia | |
| | | (megokaryoblast)_sscDNA | |
| 94910_SNB- | 0.0 | 94963_Raji_Burkitt's | 0.0 |
| 78_CNS/glioma_sscDNA | | lymphoma_sscDNA | |
| 94911_SF- | 0.0 | 94964_Daudi_Burkitt's | 0.0 |
| 268_CNS/glioblastoma_sscDNA | | lymphoma_sscDNA | |
| 94912_T98G_Glioblastoma_{—} | 0.0 | 94965_U266_B-cell | 0.0 |
| sscDNA | | plasmacytoma/myeloma_sscDNA | |
| 96776_SK-N-SH_Neuroblastoma | 0.0 | 94968_CA46_Burkitt's | 0.0 |
| (metastasis)_sscDNA | | lymphoma_sscDNA | |
| 94913_SF- | 0.0 | 94970_RL_non-Hodgkin's B-cell | 0.0 |
| 295_CNS/glioblastoma_sscDNA | | lymphoma_sscDNA | |
| 94914_Cerebellum_sscDNA | 2.4 | 94972_JM1_pre-B-cell | 0.0 |
| | | lymphoma/leukemia_sscDNA | |
| 96777_Cerebellum_sscDNA | 0.5 | 94973_Jurkat_T cell | 0.0 |
| | | leukemia_sscDNA | |
| 94916_NCI- | 0.0 | 94974_TF- | 0.0 |
| H292_Mucoepidermoid lung | | 1_Erythroleukemia_sscDNA | |
| carcinoma_sscDNA | | | |
| 94917_DMS-114_Small cell lung | 0.0 | 94975_HUT 78_T-cell | 0.0 |
| cancer_sscDNA | | lymphoma_sscDNA | |
| 94918_DMS-79_Small cell lung | 0.0 | 94977_U937_Histiocytic | 0.0 |
| cancer/neuroendocrine_sscDNA | | lymphoma_sscDNA | |
| 94919_NCI-H146_Small cell lung | 0.0 | 94980_KU-812_Myelogenous | 0.3 |
| cancer/neuroendocrine_sscDNA | | leukemia_sscDNA | |
| 94920_NCI-H526_Small cell lung | 0.0 | 94981_769-P_Clear cell renal | 0.0 |
| cancer/neuroendocrine_sscDNA | | carcinoma_sscDNA | |
| 94921_NCI-N417_Small cell lung | 0.0 | 94983_Caki-2_Clear cell renal | 0.0 |
| cancer/neuroendocrine_sscDNA | | carcinoma_sscDNA | |
| 94923_NCI-H82_Small cell lung | 0.0 | 94984_SW 839_Clear cell renal | 0.0 |
| cancer/neuroendocrine_sscDNA | | carcinoma_sscDNA | |
| 94924_NCI-H157_Squamous cell | 0.0 | 94986_G401_Wilms' | 0.0 |
| lung Cancer (metastasis)_sscDNA | | tumor_sscDNA | |
| 94925_NCI-H1155_Large cell | 0.0 | 94987_Hs766T_Pancreatic | 0.0 |
| lung | | carcinoma (LN | |
| cancer/neuroendocrine_sscDNA | | metastasis)_sscDNA | |
| 94926_NCI-H1299_Large cell | 0.0 | 94988_CAPAN-1_Pancreatic | 2.3 |
| lung | | adenocarcinoma (liver | |
| cancer/neuroendocrine_sscDNA | | metastasis)_sscDNA | |
| 94927_NCI-H727_Lung | 0.0 | 94989_SU86.86_Pancreatic | 0.2 |
| carcinoid_sscDNA | | carcinoma (liver | |
| | | metastasis)_sscDNA | |
| 94928_NCI-UMC-11_Lung | 0.3 | 94990_BxPC-3_Pancreatic | 0.3 |
| carcinoid_sscDNA | | adenocarcinoma_sscDNA | |
| 94929_LX-1_Small cell lung | 0.0 | 94991_HPAC_Pancreatic | 4.9 |
| cancer_sscDNA | | adenocarcinoma_sscDNA | |
| 94930_Colo-205_Colon | 0.0 | 94992_MIA PaCa-2_Pancreatic | 0.0 |
| cancer_sscDNA | | carcinoma_sscDNA | |
| 94931_KM12_Colon | 100.0 | 94993_CFPAC-1_Pancreatic | 0.0 |
| cancer_sscDNA | | ductal adenocarcinoma_sscDNA | |
| 94932_KM20L2_Colon | 0.0 | 94994_PANC-1_Pancreatic | 0.0 |
| cancer_sscDNA | | epithelioid ductal | |
| | | carcinoma_sscDNA | |
| 94933_NCI-H716_Colon | 0.0 | 94996_T24_Bladder carcinma | 0.0 |
| cancer_sscDNA | | (transitional cell)_sscDNA | |
| 94935_SW-48_Colon | 0.0 | 94997_5637_Bladder | 2.1 |
| adenocarcinoma_sscDNA | | carcinoma_sscDNA | |
| 94936_SW1116_Colon | 1.3 | 94998_HT-1197_Bladder | 2.6 |
| adenocarcinoma_sscDNA | | carcinoma sscDNA | |
| 94937_LS 174T_Colon | 0.0 | 94999_UM-UC-3_Bladder | 0.0 |
| adenocarcinoma_sscDNA | | carcinma (transitional | |
| | | cell)_sscDNA | |
| 94938_SW-948_Colon | 0.0 | 95000_A204_{—} | 0.0 |
| adenocarcinoma_sscDNA | | Rhabdomyosarcoma_sscDNA | |
| 94939_SW-480_Colon | 0.0 | 95001_HT- | 0.0 |
| adenocarcinoma_sscDNA | | 1080_Fibrosarcoma_sscDNA | |
| 94940_NCI-SNU-5_Gastric | 0.0 | 95002_MG-63_Osteosarcoma | 0.0 |
| carcinoma_sscDNA | | (bone)_sscDNA | |
| 94941_KATO III_Gastric | 0.0 | 95003_SK-LMS- | 0.0 |
| carcinoma_sscDNA | | 1_Leiomyosarcoma | |
| | | (vulva)_sscDNA | |
| 94943_NCI-SNU-16_Gastric | 0.0 | 95004_SJRH30_{—} | 0.0 |
| carcinoma_sscDNA | | Rhabdomyosarcoma | |
| | | (met to bone marrow)_sscDNA | |
| 94944_NCI-SNU-1_Gastric | 0.0 | 95005_A431_{—} | 0.0 |
| carcinoma_sscDNA | | Epidermoidcarcinoma_sscDNA | |
| 94946_RF-1_Gastric | 0.0 | 95007_WM266- | 0.0 |
| adenocarcinoma_sscDNA | | 4_Melanoma_sscDNA | |
| 94947_RF-48_Gastric | 0.0 | 95010_DU 145_Prostate | 0.0 |
| adenocarcinoma_sscDNA | | carcinoma (brain | |
| | | metastasis)_sscDNA | |
| 96778_MKN-45_Gastric | 0.0 | 95012_MDA-MB-468_Breast | 0.3 |
| carcinoma_sscDNA | | adenocarcinoma_sscDNA | |
| 94949_NCI-N87_Gastric | 86.5 | 95013_SCC-4_Squamous cell | 4.8 |
| carcinoma_sscDNA | | carcinoma of tongue_sscDNA | |
| 94951_OVCAR-5_Ovarian | 0.0 | 95014_SCC-9_Squamous cell | 0.0 |
| carcinoma_sscDNA | | carcinoma of tongue_sscDNA | |
| 94952_RL95-2_Uterine | 1.3 | 95015_SCC-15_Squamous cell | 0.0 |
| carcinoma_sscDNA | | carcinoma of tongue_sscDNA | |
| 94953_HelaS3_Cervical | 0.0 | 95017_CAL 27_Squamous cell | 2.4 |
| adenocarcinoma_sscDNA | | carcinoma of tongue_sscDNA | |

TABLE 19
Panel 4D/4R

| | Relative Expression(%) | | Relative Expression(%) | | |
|---|---|---|---|---|---|
| | 4dtm1922f_{—} | 4rtm1957f_{—} | 4Dtm1889f_{—} | 4Dtm1914f_{—} | 4Rtm2856f_{—} |
| Tissue Name | ag1180 | ag1180 | ag1312 | ag1312 | ag1312 |
| 93768_Secondary Th1_anti- | 0.0 | 0.0 | 0.0 | 17.8 | 0.0 |
| CD28/anti-CD3 | | | | | |
| 93769_Secondary Th2_anti- | 0.0 | 0.0 | 0.0 | 0.0 | 0.0 |
| CD28/anti-CD3 | | | | | |
| 93770_Secondary Tr1_anti- | 0.0 | 0.0 | 0.0 | 0.0 | 0.0 |
| CD28/anti-CD3 | | | | | |
| 93573_Secondary | 0.0 | 0.0 | 0.0 | 0.0 | 0.0 |
| Th1_resting day 4-6 in IL-2 | | | | | |
| 93572_Secondary | 0.0 | 0.0 | 0.0 | 0.0 | 0.0 |
| Th2_resting day 4-6 in IL-2 | | | | | |
| 93571_Secondary | 0.0 | 0.0 | 0.0 | 0.0 | 0.0 |
| Tr1_resting day 4-6 in IL-2 | | | | | |
| 93568_primary Th1_anti- | 0.0 | 0.0 | 0.0 | 0.0 | 0.0 |
| CD28/anti-CD3 | | | | | |
| 93569_primary Th2_anti- | 0.1 | 0.0 | 0.1 | 0.0 | 0.0 |
| CD28/anti-CD3 | | | | | |
| 93570_primary Tr1_anti- | 0.0 | 0.0 | 0.0 | 0.0 | 0.0 |
| CD28/anti-CD3 | | | | | |
| 93565_primary Th1_resting | 0.0 | 0.0 | 0.0 | 0.0 | 0.0 |
| dy 4-6 in IL-2 | | | | | |
| 93566_primary Th2_resting | 0.0 | 0.0 | 0.0 | 0.0 | 0.0 |
| dy 4-6 in IL-2 | | | | | |
| 93567_primary Tr1_resting | 0.0 | 0.0 | 0.0 | 0.0 | 0.0 |
| dy 4-6 in IL-2 | | | | | |
| 93351_CD45RA CD4 | 0.0 | 0.0 | 0.0 | 0.0 | 0.0 |
| lymphocyte_anti-CD28/anti- | | | | | |
| CD3 | | | | | |
| 93352_CD45RO CD4 | 0.0 | 0.0 | 0.0 | 0.0 | 0.0 |
| lymphocyte_anti-CD28/anti- | | | | | |
| CD3 | | | | | |
| 93251_CD8 | 0.0 | 0.0 | 0.0 | 0.0 | 0.0 |
| Lymphocytes_anti- | | | | | |
| CD28/anti-CD3 | | | | | |
| 93353_chronic CD8 | 0.0 | 0.0 | 0.0 | 0.0 | 0.0 |
| Lymphocytes 2ry_resting dy | | | | | |
| 4-6 in IL-2 | | | | | |
| 93574_chronic CD8 | 0.0 | 0.0 | 0.0 | 0.0 | 0.0 |
| Lymphocytes 2ry_activated | | | | | |
| CD3/CD28 | | | | | |
| 93354_CD4_none | 0.0 | 0.0 | 0.0 | 0.0 | 0.0 |
| 93252_Secondary | 0.0 | 0.0 | 0.0 | 0.0 | 0.4 |
| Th1/Th2/Tr1_anti-CD95 | | | | | |
| CH11 | | | | | |
| 93103_LAK cells_resting | 0.0 | 0.0 | 0.0 | 0.0 | 0.0 |
| 93788_LAK cells_IL-2 | 0.0 | 0.0 | 0.0 | 0.0 | 0.0 |
| 93787_LAK cells_IL-2 + IL- | 0.0 | 0.0 | 0.0 | 0.0 | 0.0 |
| 12 | | | | | |
| 93789_LAK cells_IL-2 + IFN | 0.0 | 0.0 | 0.0 | 0.0 | 0.0 |
| gamma | | | | | |
| 93790_LAK cells_IL-2 + IL- | 0.0 | 0.0 | 0.0 | 0.0 | 0.0 |
| 18 | | | | | |
| 93104_LAK | 0.0 | 0.0 | 0.0 | 0.0 | 0.0 |
| cells_PMA/ionomycin and | | | | | |
| IL-18 | | | | | |
| 93578_NK Cells IL- | 0.0 | 0.0 | 0.0 | 0.0 | 0.0 |
| 2_resting | | | | | |
| 93109_Mixed Lymphocyte | 0.0 | 0.0 | 0.0 | 0.0 | 0.0 |
| Reaction_Two Way MLR | | | | | |
| 93110_Mixed Lymphocyte | 0.0 | 0.0 | 0.0 | 0.0 | 0.0 |
| Reaction_Two Way MLR | | | | | |
| 93111_Mixed Lymphocyte | 0.0 | 0.0 | 0.0 | 0.0 | 0.0 |
| Reaction_Two Way MLR | | | | | |
| 93112_Mononuclear Cells | 0.0 | 0.0 | 0.0 | 0.0 | 0.0 |
| (PBMCs)_resting | | | | | |
| 93113_Mononuclear Cells | 0.0 | 0.0 | 0.0 | 0.0 | 0.0 |
| (PBMCs)_PWM | | | | | |
| 93114_Mononuclear Cells | 0.2 | 0.0 | 0.0 | 0.0 | 0.0 |
| (PBMCs)_PHA-L | | | | | |
| 93249_Ramos (B cell)_none | 0.0 | 0.0 | 0.0 | 0.0 | 0.0 |
| 93250_Ramos (B cell)_{—} | 0.0 | 0.0 | 0.0 | 0.0 | 0.0 |
| ionomycin | | | | | |
| 93349_B | 0.0 | 0.0 | 0.0 | 0.0 | 0.0 |
| lymphocytes_PWM | | | | | |
| 93350_B | 0.0 | 0.0 | 0.0 | 0.0 | 0.3 |
| lymphoytes_CD40L and IL-4 | | | | | |
| 92665_EOL-1 | 0.0 | 0.0 | 0.0 | 0.0 | 0.0 |
| (Eosinophil)_dbcAMP | | | | | |
| differentiated | | | | | |
| 93248_EOL-1 | 0.0 | 0.0 | 0.0 | 0.0 | 0.0 |
| (Eosinophil)_dbcAMP/PMA | | | | | |
| ionomycin | | | | | |
| 93356_Dendritic Cells_none | 0.0 | 0.0 | 0.0 | 0.0 | 0.0 |
| 93355_Dendritic Cells_LPS | 0.0 | 0.0 | 0.0 | 0.0 | 0.0 |
| 100 ng/ml | | | | | |
| 93775_Dendritic Cells_anti- | 0.0 | 0.0 | 0.0 | 0.0 | 0.0 |
| CD40 | | | | | |
| 93774_Monocytes_resting | 0.0 | 0.0 | 0.0 | 0.0 | 0.0 |
| 93776_Monocytes_LPS | 0.1 | 0.0 | 0.1 | 0.0 | 0.0 |
| 50 ng/ml | | | | | |
| 93581_Macrophages_resting | 0.0 | 0.0 | 0.0 | 0.0 | 0.0 |
| 93582_Macrophages_LPS | 0.0 | 0.0 | 0.0 | 0.0 | 0.0 |
| 100 ng/ml | | | | | |
| 93098_HUVEC | 0.0 | 0.0 | 0.0 | 0.0 | 0.0 |
| (Endothelial)_none | | | | | |
| 93099_HUVEC | 0.0 | 0.0 | 0.0 | 0.0 | 0.0 |
| (Endothelial)_starved | | | | | |
| 93100_HUVEC | 0.0 | 0.0 | 0.0 | 0.0 | 0.0 |
| (Endothelial)_IL-1b | | | | | |
| 93779_HUVEC | 0.0 | 0.0 | 0.0 | 0.0 | 0.0 |
| (Endothelial)_IFN gamma | | | | | |
| 93102_HUVEC | 0.0 | 0.0 | 0.0 | 0.0 | 0.0 |
| (Endothelial)_TNF alpha + | | | | | |
| IFN gamma | | | | | |
| 93101_HUVEC | 0.0 | 0.0 | 0.0 | 0.0 | 0.0 |
| (Endothelial)_TNF alpha + | | | | | |
| IL4 | | | | | |
| 93781_HUVEC | 0.0 | 0.0 | 0.0 | 0.0 | 0.0 |
| (Endothelial)_IL-11 | | | | | |
| 93583_Lung Microvascular | 0.0 | 0.0 | 0.0 | 0.0 | 0.0 |
| Endothelial Cells_none | | | | | |
| 93584_Lung Microvascular | 0.0 | 0.0 | 0.0 | 0.0 | 0.0 |
| Endothelial Cells_TNFa | | | | | |
| (4 ng/ml) and IL1b (1 ng/ml) | | | | | |
| 92662_Microvascular | 0.0 | 0.0 | 0.0 | 0.0 | 0.0 |
| Dermal endothelium_none | | | | | |
| 92663_Microsvasular | 0.0 | 0.0 | 0.0 | 0.0 | 0.0 |
| Dermal endothelium_TNFa | | | | | |
| (4 ng/ml) and IL1b(1 ng/ml) | | | | | |
| 93773_Bronchial | 5.3 | 2.0 | 5.7 | 3.3 | 1.7 |
| epithelium_TNFa (4 ng/ml) | | | | | |
| and IL1b (1 ng/ml)** | | | | | |
| 93347_Small Airway | 28.7 | 32.1 | 38.7 | 29.7 | 41.2 |
| Epithelium_none | | | | | |
| 93348_Small Airway | 100.0 | 100.0 | 100.0 | 100.0 | 100.0 |
| Epithelium_TNFa (4 ng/ml) | | | | | |
| and IL1b (1 ng/ml) | | | | | |
| 92668_Coronery Artery | 0.0 | 0.0 | 0.0 | 0.0 | 0.0 |
| SMC_resting | | | | | |
| 92669_Coronery Artery | 0.0 | 0.0 | 0.0 | 0.0 | 0.0 |
| SMC_TNFa (4 ng/ml) and | | | | | |
| IL1b (1 ng/ml) | | | | | |
| 93107_astrocytes_resting | 0.0 | 0.0 | 0.0 | 0.0 | 0.0 |
| 93108_astrocytes_TNFa | 0.0 | 0.0 | 0.0 | 0.0 | 0.0 |
| (4 ng/ml) and IL1b (1 ng/ml) | | | | | |
| 92666_KU-812 | 0.0 | 0.0 | 0.0 | 0.0 | 0.0 |
| (Basophil)_resting | | | | | |
| 92667_KU-812 | 0.0 | 0.0 | 0.0 | 0.1 | 0.0 |
| (Basophil)_PMA/ionoycin | | | | | |
| 93579_CCD1106 | 1.7 | 0.8 | 1.7 | 1.3 | 1.0 |
| (Keratinocytes)_none | | | | | |
| 93580_CCD1106 | 15.3 | 22.4 | 14.8 | 10.9 | 2.8 |
| (Keratinocytes)_TNFa and | | | | | |
| IFNg** | | | | | |
| 93791_Liver Cirrhosis | 0.0 | 0.0 | 0.0 | 0.0 | 0.0 |
| 93792_Lupus Kidney | 0.0 | 0.0 | 0.0 | 0.0 | 0.0 |
| 93577_NCI-H292 | 0.3 | 0.0 | 0.0 | 0.0 | 0.0 |
| 93358_NCI-H292_IL-4 | 0.3 | 0.0 | 0.0 | 0.0 | 0.3 |
| 93360_NCI-H292_IL-9 | 0.0 | 0.0 | 0.0 | 0.0 | 0.1 |
| 93359_NCI-H292_IL-13 | 0.0 | 0.0 | 0.1 | 0.0 | 0.0 |
| 93357_NCI-H292_IFN | 0.0 | 0.0 | 0.0 | 0.0 | 0.0 |
| gamma | | | | | |
| 93777_HPAEC_- | 0.0 | 0.0 | 0.0 | 0.0 | 0.0 |
| 93778_HPAEC_IL-1 | 0.0 | 0.0 | 0.0 | 0.0 | 0.0 |
| beta/TNA alpha | | | | | |
| 93254_Normal Human Lung | 0.0 | 0.0 | 0.0 | 0.0 | 0.0 |
| Fibroblast_none | | | | | |
| 93253_Normal Human Lung | 0.0 | 0.0 | 0.0 | 0.0 | 0.0 |
| Fibroblast_TNFa (4 ng/ml) | | | | | |
| and IL-1b (1 ng/ml) | | | | | |
| 93257_Normal Human Lung | 0.0 | 0.0 | 0.0 | 0.0 | 0.0 |
| Fibroblast_IL-4 | | | | | |
| 93256_Normal Human Lung | 0.0 | 0.0 | 0.0 | 0.0 | 0.0 |
| Fibroblast_IL-9 | | | | | |
| 93255_Normal Human Lung | 0.0 | 0.0 | 0.0 | 0.0 | 0.0 |
| Fibroblast_IL-13 | | | | | |
| 93258_Normal Human Lung | 0.0 | 0.0 | 0.0 | 0.0 | 0.0 |
| Fibroblast_IFN gamma | | | | | |
| 93106_Dermal Fibroblasts | 0.0 | 0.0 | 0.0 | 0.0 | 0.0 |
| CCD1070_resting | | | | | |
| 93361_Dermal Fibroblasts | 0.0 | 0.0 | 0.0 | 0.0 | 0.0 |
| CCD1070_TNF alpha | | | | | |
| 4 ng/ml | | | | | |
| 93105_Dermal Fibroblasts | 0.0 | 0.0 | 0.0 | 0.0 | 0.0 |
| CCD1070_IL-1 beta 1 ng/ml | | | | | |
| 93772_dermal | 0.0 | 0.0 | 0.0 | 0.0 | 0.0 |
| fibroblast_IFN gamma | | | | | |
| 93771_dermal | 0.0 | 0.0 | 0.0 | 0.0 | 0.0 |
| fibroblast_IL-4 | | | | | |
| 93260_IBD Colitis 2 | 0.0 | 0.0 | 0.0 | 0.0 | 0.0 |
| 93261_IBD Crohns | 0.0 | 0.0 | 0.0 | 0.0 | 0.0 |
| 735010_Colon_normal | 0.0 | 0.0 | 0.0 | 0.0 | 0.0 |
| 735019_Lung_none | 0.0 | 0.0 | 0.0 | 0.0 | 0.0 |
| 64028-1_Thymus_none | 0.1 | 0.0 | 0.2 | 0.0 | 0.0 |
| 64030-1_Kidney_none | 1.8 | 3.2 | 3.0 | 2.8 | 2.7 |

Panel 1.2 Summary: Ag1180 Results from two experiments using the same probe/primer sets are in good agreement. The NOV1 gene is most highly expressed in gastric cancer cell lines (CT=23.7, 24) and at more moderate levels in pancreatic cancer cell lines (CT=29.0, 30.7). Therefore, expression of the NOV1 gene could be used to distinguish gastric cell line derived material from other samples. In addition, these results suggest that therapeutic modulation of this gene or its protein product could be effective in the treatment of gastric cancer.

Among metabolically relevant tissues, the NOV1 gene is moderately expressed in adult skeletal muscle and adult heart tissue. (adult CT=34.2/32.8: fetal CT=39.6/40) This result suggests that the NOV1 gene, the protein encoded by NOV1 gene, or antibodies designed with the protein could be used to distinguish those tissues from the corresponding fetal tissues.

Panel 1.3D Summary: Ag1180 Moderate levels of expression of the NOV1 gene are detected in gastric cancer cell lines (CT=30.4) and lower levels in pancreatic cancer cell lines (CT=33.5). This result is consistent with the expression profile observed in Panel 1.2. See Panel 1.2 for potential utility of this gene.

Among tissues involved in central nervous system function, the NOV1 gene is specifically expressed at low to moderate levels in the amygdala, cerebellum, cortex, hippocampus and thalamus, and expressed highly in the spinal cord and cerebral cortex. Alpha-2-macroglobulin has been implicated in Alzheimer's disease, both genetically and biochemically in the clearance of beta amyloid. The high similarity of the NOV1 gene protein product to alpha-2-macroglobulin suggests probable similarity of function. Therefore, agents that affect the NOV1 gene product activity may have efficacy in treating Alzheimer's disease. If the NOV1 gene is involved in A-beta clearance, then agents that increase its expression, concentration, or activity may aid in the clearance of A-beta, which is a hallmark of Alzheimer's disease histopathology.

Panel 2D Summary: Ag1180 Expression of the NOV1 gene is highest in ovarian cancer (CT=25.6) and is overexpressed in 2/2 ovarian cancers when compared to the normal margins. Furthermore, the NOV1 gene is also overexpressed in bladder cancer, breast cancer and prostate cancer relative to the normal controls. Thus, NOV1 gene expression could be used as a marker of these cancerous tissues. In addition, therapeutic modulation of this gene product, through the use of small molecule drugs or antibodies, could be useful for the treatment of ovarian, bladder, breast and prostate cancer.

Panel 2.2 Summary: Ag1180 Expression of NOV1 is highest in bladder cancer tissue (CT=31.3) and is overexpressed in bladder cancers when compared to the normal margins. Thus, expression of the NOV1 gene could be used to distinguish bladder cancer from normal bladder tissue or other tissues. In addition, therepeutic modulation of the NOV1 gene or its protein product could potentially be useful in the treatment of bladder cancer. There is also low but significant expression of the NOV1 gene in ovarian cancer, breast cancer, and lung cancer. Thus, expression of this gene could be used to distinguish between these cancerous tissues and their normal counterparts.

Panel 3D Summary: Ag1180 The NOV1 gene is moderately expressed in colon cancer cell line (CT=29.7) and gastric cancer cell line (CT=29.9) and expressed at low levels in pancreatic cancer cell line (CT=34). These results are consistent with the expression patterns observed in panels 1.2 and 1.3D. Thus, expression of this gene could be used to distinguish colon and stomach cancers from other tissues.

Panel 4D/4R Summary: Ag1180/Ag1312 Five experiments using the same probe/primer set show results that are in excellent agreement. Expression of the NOV1 gene is detected at moderate levels in small airway epithelium (CT=28) and is slightly upregulated when treated with TNF-alpha+IL-1beta (CT=26-27). The NOV1 gene encodes a protein that is most likely a macroglobulin-like molecule belonging to a class of proteinase inhibitor that can behave as a potent modulator of the inflammatory reaction and tissue repair mechanism. Therefore, protein therapeutics designed against the NOV1 gene product could modulate the inflammatory responses observed in asthma, emphysema. In addition, the presence of expression in keratinocytes stimulated with the inflammatory cytokines TNF-alpha+IL-1beta (CT=29) suggests potential utility of the NOV1 gene product in skin related disease such as psoriasis, eczema, and contact dermatitis. Since this class of protein can in some situations act as acute phase protein, antibody targets against the protein encoded by the NOV1 gene might also be useful against the previously mentioned diseases. (Allgayer et al., Clin Exp Metastasis 16(1):62-73, 1998; Khalifa et al., Chemioterapia 6:736-7, 1987; Blacker et al., Nat Genet 19:357-60, 1998; Mikhailenko et al., J Biol Chem. Aug. 15, 2001.)

NOV2: Secreted Proteins Related to Angiogenesis

Expression of the NOV2 gene (AC005799_A) was assessed using the primer-probe set Ag1385, described in Table 20. Results from RTQ-PCR runs are shown in Tables 21, 22, and 23.

TABLE 20

Probe Name Ag1385

| | | | | | |
|---|---|---|---|---|---|
| | | | | Start | |
| Primers | Sequences | TM | Length | Position | SEQ ID NO: |

| | | | | | |
|---|---|---|---|---|---|
| Forward | 5′-AGGTCACCAAGAATGAAATCCT-3′ | 59 | 22 | 278 | 152 |
| Probe | FAM-5′- | 69.2 | 26 | 306 | 153 |
| | TGTTTTCTTTGTCTCTCCAGCGAGCA-3′ | | | | |
| | TAMRA | | | | |
| Reverse | 5′-CTTGCACATGTATGGACACTTG-3′ | 59.1 | 22 | 348 | 154 |

TABLE 21
Panel 1.2

| | Relative Expression (%) | |
|---|---|---|
| | 1.2tm1609f_{—} | 1.2tm1812f_{—} |
| Tissue Name | ag1385 | ag1385 |
| Endothelial cells | 0.0 | 0.0 |
| Heart (fetal) | 16.8 | 8.6 |
| Pancreas | 0.0 | 0.2 |
| Pancreatic ca. CAPAN 2 | 0.0 | 0.0 |
| Adrenal Gland (new lot*) | 1.4 | 3.3 |
| Thyroid | 0.3 | 0.4 |
| Salivary gland | 100.0 | 100.0 |
| Pituitary gland | 0.8 | 1.1 |
| Brain (fetal) | 0.5 | 0.2 |
| Brain (whole) | 0.5 | 0.2 |
| Brain (amygdala) | 0.8 | 0.4 |
| Brain (cerebellum) | 0.2 | 0.2 |
| Brain (hippocampus) | 2.4 | 1.1 |
| Brain (thalamus) | 12.5 | 6.8 |
| Cerebral Cortex | 3.3 | 1.4 |
| Spinal cord | 1.7 | 2.8 |
| CNS ca. (glio/astro) U87-MG | 0.0 | 0 |
| CNS ca. (glio/astro) U-118-MG | 0.5 | 0.5 |
| CNS ca. (astro) SW1783 | 0.2 | 0.1 |
| CNS ca.* (neuro; met) SK-N-AS | 0.0 | 0.0 |
| CNS ca. (astro) SF-539 | 0.0 | 0.0 |
| CNS ca. (astro) SNB-75 | 0.5 | 0.4 |
| CNS ca. (glio) SNB-19 | 0.0 | 0.0 |
| CNS ca. (glio) U251 | 0.0 | 0.0 |
| CNS ca. (glio) SF-295 | 1.8 | 1.8 |
| Heart | 6.3 | 16.5 |
| Skeletal Muscle (new lot*) | 2.4 | 5.4 |
| Bone marrow | 1.1 | 0.8 |
| Thymus | 1.0 | 0.3 |
| Spleen | 1.3 | 0.9 |
| Lymph node | 2.6 | 0.6 |
| Colorectal | 0.6 | 0.4 |
| Stomach | 8.5 | 1.7 |
| Small intestine | 6.9 | 2.8 |
| Colon ca. SW480 | 0.0 | 0.0 |
| Colon ca.* (SW480 met) SW620 | 0.0 | 0.0 |
| Colon ca. HT29 | 0.0 | 0.0 |
| Colon ca. HCT-116 | 0.0 | 0.0 |
| Colon ca. CaCo-2 | 0.2 | 0.3 |
| 83219 CC Well to Mod Diff | 1.4 | 0.8 |
| (ODO3866) | | |
| Colon ca. HCC-2998 | 0.0 | 0.0 |
| Gastric ca.* (liver met) NCI-N87 | 0.0 | 0.0 |
| Bladder | 29.9 | 20.9 |
| Trachea | 3.6 | 2.5 |
| Kidney | 35.1 | 25.5 |
| Kidney (fetal) | 4.8 | 2.9 |
| Renal ca. 786-0 | 0.0 | 0.0 |
| Renal ca. A498 | 0.0 | 0.0 |
| Renal ca. RXF 393 | 0.0 | 0.0 |
| Renal ca. ACHN | 0.0 | 0.0 |
| Renal ca. UO-31 | 0.0 | 0.0 |
| Renal ca. TK-10 | 0.0 | 0.0 |
| Liver | 37.4 | 28.7 |
| Liver (fetal) | 16.6 | 12 |
| Liver ca. (hepatoblast) HepG2 | 0.0 | 0.0 |
| Lung | 2.5 | 0.5 |
| Lung (fetal) | 14.1 | 5.7 |
| Lung ca. (small cell) LX-1 | 0.0 | 0.0 |
| Lung ca. (small cell) NCI-H69 | 0.5 | 0.3 |
| Lung ca. (s.cell var.) SHP-77 | 0.0 | 0.0 |
| Lung ca. (large cell) NCI-H460 | 0.7 | 0.2 |
| Lung ca. (non-sm. cell) A549 | 0.0 | 0.0 |
| Lung ca. (non-s.cell) NCI-H23 | 0.4 | 0.3 |
| Lung ca (non-s.cell) HOP-62 | 0.1 | 0.2 |
| Lung ca. (non-s.cl) NCI-H522 | 0.0 | 0.0 |
| Lung ca. (squam.) SW 900 | 16.5 | 10.4 |
| Lung ca. (squam.) NCI-H596 | 2.5 | 1.8 |
| Mammary gland | 11.8 | 6.2 |
| Breast ca.* (pl. effusion) MCF-7 | 0.0 | 0.0 |
| Breast ca.* (pl.ef) MDA-MB-231 | 0.0 | 0.0 |
| Breast ca.* (pl. effusion) T47D | 0.0 | 0.0 |
| Breast ca. BT-549 | 0.5 | 0.4 |
| Breast ca. MDA-N | 0.0 | 0.0 |
| Ovary | 21.5 | 11.7 |
| Ovarian ca. OVCAR-3 | 0.2 | 0.2 |
| Ovarian ca. OVCAR-4 | 10.9 | 7.6 |
| Ovarian ca. OVCAR-5 | 0.5 | 0.3 |
| Ovarian ca. OVCAR-8 | 0.0 | 0.0 |
| Ovarian ca. IGROV-1 | 1.3 | 0.9 |
| Ovarian ca.* (ascites) SK-OV-3 | 2.8 | 2.1 |
| Uterus | 9.0 | 6.2 |
| Placenta | 4.2 | 0.6 |
| Prostate | 3.1 | 1.6 |
| Prostate ca.* (bone met) PC-3 | 1.7 | 1.9 |
| Testis | 1.6 | 1.8 |
| Melanoma Hs688 (A).T | 0.3 | 0.2 |
| Melanoma* (met) Hs688 (B).T | 1.6 | 1.3 |
| Melanoma UACC-62 | 0.0 | 0.0 |
| Melanoma M14 | 0.0 | 0.0 |
| Melanoma LOX IMVI | 0.0 | 0.0 |
| Melanoma* (met) SK-MEL-5 | 0.0 | 0.0 |

TABLE 22
Panel 2D

| | Relative Expression (%) | |
|---|---|---|
| | 2Dtm2327f_{—} | 2Dtm3180f_{—} |
| Tissue Name | ag1385 | ag1385 |
| Normal Colon GENPAK 061003 | 9.4 | 10.0 |
| 83219 CC Well to Mod Diff (ODO3866) | 6.1 | 5.6 |
| 83220 CC NAT (ODO3866) | 2.0 | 1.1 |
| 83221 CC Gr.2 rectosigmoid (ODO3868) | 2.3 | 1.8 |
| 83222 CC NAT (ODO3868) | 0.9 | 1.1 |
| 83235 CC Mod Diff (ODO3920) | 1.1 | 1.9 |
| 83236 CC NAT (ODO3920) | 2.5 | 2.8 |
| 83237 CC Gr.2 ascend colon (ODO3921) | 3.4 | 4.5 |
| 83238 CC NAT (ODO3921) | 1.6 | 2.4 |
| 83241 CC from Partial Hepatectomy | 10.5 | 14.8 |
| (ODO4309) | | |
| 83242 Liver NAT (ODO4309) | 55.5 | 66.4 |
| 87472 Colon mets to lung (OD04451-01) | 20.7 | 14.7 |
| 87473 Lung NAT (OD04451-02) | 25.9 | 20.2 |
| Normal Prostate Clontech A + 6546-1 | 3.5 | 1.9 |
| 84140 Prostate Cancer (OD04410) | 7.7 | 5.3 |
| 84141 Prostate NAT (OD04410) | 8.2 | 9.9 |
| 87073 Prostate Cancer (OD04720-01) | 3.4 | 2.3 |
| 87074 Prostate NAT (OD04720-02) | 8.5 | 8.7 |
| Normal Lung GENPAK 061010 | 34.9 | 34.2 |
| 83239 Lung Met to Muscle (ODO4286) | 22.1 | 33.9 |
| 83240 Muscle NAT (ODO4286) | 54.7 | 29.5 |
| 84136 Lung Malignant Cancer (OD03126) | 55.9 | 36.9 |
| 84137 Lung NAT (OD03126) | 64.6 | 66.0 |
| 84871 Lung Cancer (OD04404) | 37.1 | 46.7 |
| 84872 Lung NAT (OD04404) | 82.4 | 44.4 |
| 84875 Lung Cancer (OD04565) | 13.1 | 10.8 |
| 84876 Lung NAT (OD04565) | 23.2 | 18.8 |
| 85950 Lung Cancer (OD04237-01) | 27.2 | 16.3 |
| 85970 Lung NAT (OD04237-02) | 52.8 | 32.8 |
| 83255 Ocular Mel Met to Liver (ODO4310) | 1.1 | 0.6 |
| 83256 Liver NAT (ODO4310) | 78.5 | 39.8 |
| 84139 Melanoma Mets to Lung (OD04321) | 2.0 | 2.0 |
| 84138 Lung NAT (OD04321) | 55.1 | 31.9 |
| Normal Kidney GENPAK 061008 | 17.4 | 13.9 |
| 83786 Kidney Ca, Nuclear grade 2 | 88.9 | 99.3 |
| (OD04338) | | |
| 83787 Kidney NAT (OD04338) | 20.9 | 21.0 |
| 83788 Kidney Ca Nuclear grade 1/2 | 27.5 | 25.2 |
| (OD04339) | | |
| 83789 Kidney NAT (OD04339) | 28.1 | 20.6 |
| 83790 Kidney Ca, Clear cell type | 14.6 | 12.0 |
| (OD04340) | | |
| 83791 Kidney NAT (OD04340) | 36.6 | 24.3 |
| 83792 Kidney Ca, Nuclear grade 3 | 15.7 | 7.9 |
| (OD04348) | | |
| 83793 Kidney NAT (OD04348) | 50.3 | 35.4 |
| 87474 Kidney Cancer (OD04622-01) | 85.3 | 65.5 |
| 87475 Kidney NAT (OD04622-03) | 7.1 | 6.3 |
| 85973 Kidney Cancer (OD04450-01) | 9.0 | 5.5 |
| 85974 Kidney NAT (OD04450-03) | 27.4 | 18.8 |
| Kidney Cancer Clontech 8120607 | 4.7 | 2.4 |
| Kidney NAT Clontech 8120608 | 24.8 | 10.1 |
| Kidney Cancer Clontech 8120613 | 1.6 | 0.4 |
| Kidney NAT Clontech 8120614 | 13.6 | 14.4 |
| Kidney Cancer Clontech 9010320 | 100.0 | 94.0 |
| Kidney NAT Clontech 9010321 | 77.9 | 49.0 |
| Normal Uterus GENPAK 061018 | 16.8 | 10.7 |
| Uterus Cancer GENPAK 064011 | 64.6 | 40.3 |
| Normal Thyroid Clontech A + 6570-1 | 16.7 | 4.6 |
| Thyroid Cancer GENPAK 064010 | 29.5 | 18.0 |
| Thyroid Cancer INVITROGEN A302152 | 94.6 | 66.0 |
| Thyroid NAT INVITROGEN A302153 | 9.2 | 6.0 |
| Normal Breast GENPAK 061019 | 30.1 | 15.1 |
| 84877 Breast Cancer (OD04566) | 7.2 | 4.0 |
| 85975 Breast Cancer (OD04590-01) | 8.0 | 4.7 |
| 85976 Breast Cancer Mets (OD04590-03) | 11.0 | 5.0 |
| 87070 Breast Cancer Metastasis | 5.9 | 3.9 |
| (OD04655-05) | | |
| GENPAK Breast Cancer 064006 | 14.4 | 8.2 |
| Breast Cancer Res. Gen. 1024 | 27.0 | 18.7 |
| Breast Cancer Clontech 9100266 | 9.3 | 7.6 |
| Breast NAT Clontech 9100265 | 25.5 | 18.2 |
| Breast Cancer INVITROGEN A209073 | 15.0 | 13.4 |
| Breast NAT INVITROGEN A2090734 | 32.3 | 17.7 |
| Normal Liver GENPAK 061009 | 58.2 | 45.1 |
| Liver Cancer GENPAK 064003 | 42.0 | 31.9 |
| Liver Cancer Research Genetics RNA 1025 | 44.1 | 40.9 |
| Liver Cancer Research Genetics RNA 1026 | 85.3 | 81.8 |
| Paired Liver Cancer Tissue Research | 86.5 | 60.7 |
| Genetics RNA 6004-T | | |
| Paired Liver Tissue Research Genetics | 37.9 | 31.4 |
| RNA 6004-N | | |
| Paired Liver Cancer Tissue Research | 92.0 | 77.9 |
| Genetics RNA 6005-T | | |
| Paired Liver Tissue Research | 25.3 | 18.3 |
| Genetics RNA 6005-N | | |
| Normal Bladder GENPAK 061001 | 18.7 | 19.2 |
| Bladder Cancer Research Genetics | 4.0 | 4.3 |
| RNA 1023 | | |
| Bladder Cancer INVITROGEN A302173 | 8.0 | 4.9 |
| 87071 Bladder Cancer (OD04718-01) | 9.4 | 8.0 |
| 87072 Bladder Normal Adjacent | 41.2 | 25.5 |
| (OD04718-03) | | |
| Normal Ovary Res. Gen. | 10.0 | 9.3 |
| Ovarian Cancer GENPAK 064008 | 76.8 | 100.0 |
| 87492 Ovary Cancer (OD04768-07) | 54.7 | 62.0 |
| 87493 Ovary NAT (OD04768-08) | 16.7 | 14.4 |
| Normal Stomach GENPAK 061017 | 17.1 | 20.3 |
| Gastric Cancer Clontech 9060358 | 2.2 | 1.2 |
| NAT Stomach Clontech 9060359 | 11.6 | 11.6 |
| Gastric Cancer Clontech 9060395 | 7.2 | 5.1 |
| NAT Stomach Clontech 9060394 | 10.4 | 9.5 |
| Gastric Cancer Clontech 9060397 | 7.3 | 9.0 |
| NAT Stomach Clontech 9060396 | 8.5 | 6.6 |
| Gastric Cancer GENPAK 064005 | 5.7 | 4.9 |

TABLE 23
Panel 4D/4R

| | Relative Expression (%) | |
|---|---|---|
| | 4dtm1923f_{—} | 4rtm1959f_{—} |
| Tissue Name | ag1385 | ag1385 |
| 93768_Secondary Th1_anti-CD28/anti-CD3 | 0.0 | 0.0 |
| 93769_Secondary Th2_anti-CD28/anti-CD3 | 0.0 | 0.0 |
| 93770_Secondary Tr1_anti-CD28/anti-CD3 | 0.0 | 0.0 |
| 93573_Secondary Th1_resting day 4-6 | 0.0 | 0.0 |
| in IL-2 | | |
| 93572_Secondary Th2_resting day 4-6 | 0.0 | 0.0 |
| in IL-2 | | |
| 93571_Secondary Tr1_resting day 4-6 | 0.0 | 0.0 |
| in IL-2 | | |
| 93568_primary Th1_anti-CD28/anti-CD3 | 0.0 | 0.0 |
| 93569_primary Th2_anti-CD28/anti-CD3 | 0.0 | 0.0 |
| 93570_primary Tr1_anti-CD28/anti-CD3 | 0.0 | 0.0 |
| 93565_primary Th1_resting dy 4-6 | 0.0 | 0.0 |
| in IL-2 | | |
| 93566_primary Th2_resting dy 4-6 | 0.0 | 0.0 |
| in IL-2 | | |
| 93567_primary Tr1_resting dy 4-6 | 0.0 | 0.0 |
| in IL-2 | | |
| 93351_CD45RA CD4 lymphocyte_{—} | 0.6 | 0.6 |
| anti-CD28/anti-CD3 | | |
| 93352_CD45RO CD4 lymphocyte_{—} | 0.0 | 0.0 |
| anti-CD28/anti-CD3 | | |
| 93251_CD8 Lymphocytes_{—} | 0.0 | 0.1 |
| anti-CD28/anti-CD3 | | |
| 93353_chronic CD8 Lymphocytes 2ry_{—} | 0.0 | 0.0 |
| resting dy 4-6 in IL-2 | | |
| a93574_chronic CD8 Lymphocytes 2ry_{—} | 0.0 | 0.0 |
| activated CD3/CD28 | | |
| 93354_CD4_none | 0.0 | 0.1 |
| 93252_Secondary Th1/Th2/Tr1_{—} | 0.0 | 0.0 |
| anti-CD95 CH11 | | |
| 93103_LAK cells_resting | 62.4 | 65.1 |
| 93788_LAK cells_IL-2 | 0.4 | 0.2 |
| 93787_LAK cells_IL-2 + IL-12 | 4.6 | 7.0 |
| 93789_LAK cells_IL-2 + IFN gamma | 4.6 | 8.5 |
| 93790_LAK cells_IL-2 + IL-18 | 3.6 | 5.7 |
| 93104_LAK cells_PMA/ionomycin | 14.9 | 17.6 |
| and IL-18 | | |
| 93578_NK Cells IL-2_resting | 0.0 | 0.0 |
| 93109_Mixed Lymphocyte Reaction_{—} | 18.3 | 16.4 |
| Two Way MLR | | |
| 93110_Mixed Lymphocyte Reaction_{—} | 14.2 | 21.3 |
| Two Way MLR | | |
| 93111_Mixed Lymphocyte Reaction_{—} | 3.7 | 4.0 |
| Two Way MLR | | |
| 93112_Mononuclear Cells (PBMCs)_{—} | 0.4 | 0.3 |
| resting | | |
| 93113_Mononuclear Cells (PBMCs)_PWM | 3.2 | 5.9 |
| 93114_Mononuclear Cells (PBMCs)_{—} | 26.1 | 28.5 |
| PHA-L | | |
| 93249_Ramos (B cell)_none | 0.0 | 0.0 |
| 93250_Ramos (B cell)_ionomycin | 0.0 | 0.0 |
| 93349_B lymphocytes_PWM | 0.0 | 0.0 |
| 93350_B lymphoytes_{—} | 0.2 | 0.4 |
| CD40L and IL-4 | | |
| 92665_EOL-1 (Eosinophil)_{—} | 0.0 | 0.0 |
| dbcAMP differentiated | | |
| 93248_EOL-1 (Eosinophil)_{—} | 0.0 | 0.0 |
| dbcAMP/PMAionomycin | | |
| 93356_Dendritic Cells_none | 31.0 | 41.5 |
| 93355_Dendritic Cells_LPS 100 ng/ml | 80.1 | 91.4 |
| 93775_Dendritic Cells_anti-CD40 | 49.7 | 57.8 |
| 93774_Monocytes_resting | 0.7 | 0.2 |
| 93776_Monocytes_LPS 50 ng/ml | 29.7 | 37.9 |
| 93581_Macrophages_resting | 20.7 | 15.2 |
| 93582_Macrophages_LPS 100 ng/ml | 100.0 | 100.0 |
| 93098_HUVEC (Endothelial)_none | 0.0 | 0.0 |
| 93099_HUVEC (Endothelial)_starved | 0.1 | 0.0 |
| 93100_HUVEC (Endothelial)_IL-1b | 0.0 | 0.0 |
| 93779_HUVEC (Endothelial)_IFN gamma | 0.0 | 0.0 |
| 93102_HUVEC (Endothelial)_TNF | 0.0 | 0.0 |
| alpha + IFN gamma | | |
| 93101_HUVEC (Endothelial)_{—} | 0.0 | 0.0 |
| TNF alpha + IL4 | | |
| 93781_HUVEC (Endothelial)_IL-11 | 0.0 | 0.0 |
| 93583_Lung Microvascular Endothelial | 0.0 | 0.0 |
| Cells_none | | |
| 93584_Lung Microvascular Endothelial | 0.0 | 0.0 |
| Cells_TNFa (4 ng/ml) and IL1b (1 ng/ml) | | |
| 92662_Microvascular Dermal | 0.0 | 0.0 |
| endothelium_none | | |
| 92663_Microsvasular Dermal endothelium_{—} | 0.0 | 0.0 |
| TNFa (4 ng/ml) and IL1b (1 ng/ml) | | |
| 93773_Bronchial epithelium_{—} | 0.3 | 0.2 |
| TNFa (4 ng/ml) and IL1b (1 ng/ml)** | | |
| 93347_Small Airway Epithelium_none | 1.2 | 0.3 |
| 93348_Small Airway Epithelium_{—} | 1.4 | 3.1 |
| TNFa (4 ng/ml) and IL1b (1 ng/ml) | | |
| 92668_Coronery Artery SMC_resting | 0.0 | 0.4 |
| 92669_Coronery Artery SMC_{—} | 0.0 | 0.2 |
| TNFa (4 ng/ml) and IL1b (1 ng/ml) | | |
| 93107_astrocytes_resting | 0.0 | 0.0 |
| 93108_astrocytes_{—} | 0.0 | 0.0 |
| TNFa (4 ng/ml) and IL1b (1 ng/ml) | | |
| 92666_KU-812 (Basophil)_resting | 0.0 | 0.0 |
| 92667_KU-812 (Basophil)_PMA/ionoycin | 0.0 | 0.0 |
| 93579_CCD1106 (Keratinocytes)_none | 0.0 | 0.0 |
| 93580_CCD1106 (Keratinocytes)_{—} | 0.0 | 0.0 |
| TNFa and IFNg** | | |
| 93791_Liver Cirrhosis | 12.9 | 23.5 |
| 93792_Lupus Kidney | 17.6 | 25.9 |
| 93577_NCI-H292 | 0.0 | 0.0 |
| 93358_NCI-H292_IL-4 | 0.0 | 0.2 |
| 93360_NCI-H292_IL-9 | 0.2 | 0.0 |
| 93359_NCI-H292_IL-13 | 0.0 | 0.2 |
| 93357_NCI-H292_IFN gamma | 0.0 | 0.0 |
| 93777_HPAEC_- | 0.0 | 0.0 |
| 93778_HPAEC_IL-1 beta/TNA alpha | 0.0 | 0.0 |
| 93254_Normal Human Lung | 0.3 | 0.8 |
| Fibroblast_none | | |
| 93253_Normal Human Lung | 0.5 | 0.9 |
| Fibroblast_TNFa (4 ng/ml) | | |
| and IL-1b (1 ng/ml) | | |
| 93257_Normal Human Lung | 0.5 | 0.8 |
| Fibroblast_IL-4 | | |
| 93256_Normal Human Lung | 0.0 | 0.7 |
| Fibroblast_IL-9 | | |
| 93255_Normal Human Lung | 1.1 | 0.0 |
| Fibroblast_IL-13 | | |
| 93258_Normal Human Lung | 2.3 | 1.9 |
| Fibroblast_IFN gamma | | |
| 93106_Dermal Fibroblasts | 6.0 | 6.4 |
| CCD1070_resting | | |
| 93361_Dermal Fibroblasts CCD1070_TNF | 4.1 | 4.3 |
| alpha 4 ng/ml | | |
| 93105_Dermal Fibroblasts CCD1070_{—} | 3.8 | 3.8 |
| IL-1 beta 1 ng/ml | | |
| 93772_dermal fibroblast_IFN gamma | 62.8 | 90.1 |
| 93771_dermal fibroblast_IL-4 | 15.8 | 8.2 |
| 93260_IBD Colitis 2 | 0.9 | 0.5 |
| 93261_IBD Crohns | 0.5 | 2.4 |
| 735010_Colon_normal | 1.7 | 4.3 |
| 735019_Lung_none | 45.4 | 75.3 |
| 64028-1_Thymus_none | 35.1 | 25.7 |
| 64030-1_Kidney_none | 7.1 | 14.8 |

Panel 1.2 Summary: Ag1385 Results from two experiments using the same probe/primer set are in very good agreement. The NOV2 gene is expressed in high to moderate levels across a wide variety of tissues. In this panel, expression of the NOV2 gene appears to be generally restricted to normal tissue as compared to cultured cancer cell lines. The NOV2 gene is most highly expressed in the salivary gland, liver, kidney, bladder, stomach and small intestine. Based on its homology to well characterized secreted molecules, the NOV2 gene product may be useful as a protein or antibody target for diseases involving any or all of these tissues.

The NOV2 gene is widely expressed in tissues involved in central nervous system function, including the amygdala (CT=30), cerebellum (CT=32), hippocampus (CT=28), thalamus (CT=26), cerebral cortex (CT=28), spinal cord (CT=27-29), cerebellum, substantia nigra and the developing brain. There is considerable evidence that angiogenesis occurs in response to ischemic stroke, and that re-vascularization occurs as part of the CNS healing process. Since the NOV2 gene is predicted to be involved in angiogenesis, therapeutic up-regulation of this gene or its protein product may therefore facilitate or enhance the recovery process in the days following stroke.

Panel 2D Summary: Ag1385 Results from two experiments using the same probe/primer set are in very good agreement. The NOV2 gene is expressed across a wide variety of tissue samples, with highest expression seen in normal kidney and ovarian cancer (CT=25). In particular, there is substantial overexpression of this gene in ovarian cancer when compared to normal ovarian tissue. Thus, this gene could potentially be used to distinguish ovarian cancer from normal ovarian tissue. In addition, therapeutic modulation of the NOV2 gene or its protein product could be useful in the treatment of ovarian cancer.

Panel 4D/4R Summary: Ag1385 Results from two experiments using the same probe/primer set are in excellent agreement. Expression of the NOV2 gene is highest in LPS treated macrophages and dendritic cells (CTs=29.7/27.7). The NOV2 gene is also expressed at moderate levels in LPS treated monocytes and dermal fibroblasts stimulated with IFN gamma. The NOV2 gene most likely encodes a novel uncharacterized secreted protein that could be a potential protein or antibody target used in modulating the inflammatory response in immune mediated diseases such as rheumatid arthritis (RA), inflammatory bowel disease (IBD), lung inflammatory diseases and infectious diseases. In addition, the presence of the NOV2 gene in activated dermal fibroblasts suggests a potential use for NOV2 protein product in the treatment of psoriasis and other related inflammatory skin diseases. (Wei et al., Collateral growth and angiogenesis around cortical stroke. Stroke 32:2179-84. 2001; Cheung et al., Induction of angiogenesis related genes in the contralateral cortex with a rat three-vessel occlusion model. Chin J Physiol 43:119-24, 2000; Marti et al., Am J Pathol. 156:965-76, 2000)

NOV3: Leucine Rich-like

Expression of the NOV3 gene (SC124141642_A) was assessed using the primer-probe sets Ag1388 and Ag2455, described in Tables 24 and 25. Results of the RTQ-PCR runs are shown in Tables 26, 27, 28, 29 and 30.

TABLE 24

Probe Name Ag1388

| | | | | | |
|---|---|---|---|---|---|
| Primers | Sequences | TM | Length | Start Position | SEQ ID NO: |

| | | | | | |
|---|---|---|---|---|---|
| Forward | 5′-CTGGTAATCCTGCTGGACTACA-3′ | 59.3 | 22 | 412 | 155 |
| Probe | FAM-5′- | 69.5 | 23 | 434 | 156 |
| | CTTTCCAGGACCTGCACAGCCTG-3′ | | | | |
| | TAMRA | | | | |
| Reverse | 5′-AGACGAATACCAGGTCGTTGT-3′ | 58.6 | 21 | 476 | 157 |

TABLE 25

Probe Name Ag2455

| | | | | | |
|---|---|---|---|---|---|
| | | | | Start | |
| Primers | Sequences | TM | Length | Position | SEQ ID NO: |

| | | | | | |
|---|---|---|---|---|---|
| Forward | 5′-GCTGGTAATCCTGCTGGACTA-3′ | 59.3 | 21 | 475 | 158 |
| Probe | FAN-5′- | 69.9 | 24 | 497 | 159 |
| | ACTTTCCAGGACCTGCACAGCCTG-3′- | | | | |
| | TAMRA | | | | |
| Reverse | 5′-AGACGAATACCAGGTCGTTGT-3′ | 58.6 | 21 | 540 | 160 |

TABLE 26
Panel 1.2

| | Relative | | Relative |
|---|---|---|---|
| | Expression (%) | | Expression (%) |
| | 1.2tm1617f_{—} | | 1.2tm1617f_{—} |
| Tissue Name | ag1388 | Tissue Name | ag1388 |
| Endothelial cells | 0.9 | Renal ca. 786-0 | 0.3 |
| Heart (fetal) | 0.7 | Renal ca. A498 | 0.2 |
| Pancreas | 0.2 | Renal ca. RXF 393 | 1.7 |
| Pancreatic ca. CAPAN 2 | 0.2 | Renal ca. ACHN | 0.0 |
| Adrenal Gland (new lot*) | 6.1 | Renal ca. UO-31 | 0.2 |
| Thyroid | 0.9 | Renal ca. TK-10 | 0.0 |
| Salivary gland | 18.3 | Liver | 1.7 |
| Pituitary gland | 0.0 | Liver (fetal) | 2.0 |
| Brain (fetal) | 1.1 | Liver ca. (hepatoblast) HepG2 | 2.9 |
| Brain (whole) | 10.5 | Lung | 1.6 |
| Brain (amygdala) | 7.4 | Lung (fetal) | 0.4 |
| Brain (cerebellum) | 100.0 | Lung ca. (small cell) LX-1 | 1.3 |
| Brain (hippocampus) | 12.4 | Lung ca. (small cell) NCI-H69 | 6.6 |
| Brain (thalamus) | 20.2 | Lung ca. (s.cell var.) SHP-77 | 0.0 |
| Cerebral Cortex | 20.4 | Lung ca. (large cell) NCI-H460 | 0.9 |
| Spinal cord | 1.6 | Lung ca. (non-sm. cell) A549 | 2.7 |
| CNS ca. (glio/astro) U87-MG | 3.3 | Lung ca. (non-s.cell) NCI-H23 | 0.3 |
| CNS ca. (glio/astro) U-118-MG | 3.4 | Lung ca (non-s.cell) HOP-62 | 0.0 |
| CNS ca. (astro) SW1783 | 1.1 | Lung ca. (non-s.cl) NCI-H522 | 0.5 |
| CNS ca.* (neuro; met) SK-N-AS | 2.5 | Lung ca. (squam.) SW 900 | 1.3 |
| CNS ca. (astro) SF-539 | 1.3 | Lung ca. (squam.) NCI-H596 | 3.3 |
| CNS ca. (astro) SNB-75 | 1.7 | Mammary gland | 3.3 |
| CNS ca. (glio) SNB-19 | 0.7 | Breast ca.* (pl. effusion) MCF-7 | 6.3 |
| CNS ca. (glio) U251 | 0.0 | Breast ca.* (pl.ef) MDA-MB-231 | 0.2 |
| CNS ca. (glio) SF-295 | 0.2 | Breast ca.* (pl. effusion) T47D | 15.6 |
| Heart | 1.0 | Breast ca. BT-549 | 0.5 |
| Skeletal Muscle (new lot*) | 0.2 | Breast ca. MDA-N | 0.3 |
| Bone marrow | 38.4 | Ovary | 0.6 |
| Thymus | 0.9 | Ovarian ca. OVCAR-3 | 2.9 |
| Spleen | 6.8 | Ovarian ca. OVCAR-4 | 0.7 |
| Lymph node | 6.0 | Ovarian ca. OVCAR-5 | 1.3 |
| Colorectal | 0.5 | Ovarian ca. OVCAR-8 | 1.1 |
| Stomach | 60.7 | Ovarian ca. IGROV-1 | 5.7 |
| Small intestine | 5.9 | Ovarian ca.* (ascites) SK-OV-3 | 3.9 |
| Colon ca. SW480 | 0.1 | Uterus | 2.4 |
| Colon ca.* (SW480 met) SW620 | 1.2 | Placenta | 1.8 |
| Colon ca. HT29 | 0.2 | Prostate | 2.4 |
| Colon ca. HCT-116 | 0.4 | Prostate ca* (bone met) PC-3 | 0.3 |
| Colon ca. CaCo-2 | 1.1 | Testis | 4.2 |
| 83219 CC Well to Mod Diff | 2.7 | Melanoma Hs688 (A).T | 0.2 |
| (ODO3866) | | | |
| Colon ca. HCC-2998 | 6.9 | Melanoma* (met) Hs688 (B).T | 0.7 |
| Gastric ca.* (liver met) NCI-N87 | 0.5 | Melanoma UACC-62 | 0.1 |
| Bladder | 9.7 | Melanoma M14 | 0.0 |
| Trachea | 4.8 | Melanoma LOX IMVI | 0.0 |
| Kidney | 0.8 | Melanoma* (met) SK-MEL-5 | 0.0 |
| Kidney (fetal) | 0.4 | | |

TABLE 27
Panel 1.3D

| | Relative | | Relative |
|---|---|---|---|
| | Expression (%) | | Expression (%) |
| | 1.3dtm4554f_{—} | | 1.3dtm4554f_{—} |
| Tissue Name | ag2455 | Tissue Name | ag2455 |
| Liver adenocarcinoma | 0.0 | Kidney (fetal) | 0.0 |
| Pancreas | 1.4 | Renal ca. 786-0 | 0.0 |
| Pancreatic ca. CAPAN 2 | 0.0 | Renal ca. A498 | 0.0 |
| Adrenal gland | 6.0 | Renal ca. RXF 393 | 1.2 |
| Thyroid | 2.0 | Renal ca. ACHN | 0.0 |
| Salivary gland | 1.8 | Renal ca. UO-31 | 0.0 |
| Pituitary gland | 1.4 | Renal ca. TK-10 | 0.0 |
| Brain (fetal) | 5.1 | Liver | 0.0 |
| Brain (whole) | 32.3 | Liver (fetal) | 4.9 |
| Brain (amygdala) | 50.7 | Liver ca. (hepatoblast) HepG2 | 2.0 |
| Brain (cerebellum) | 84.1 | Lung | 10.3 |
| Brain (hippocampus) | 72.7 | Lung (fetal) | 0.2 |
| Brain (substantia nigra) | 7.2 | Lung ca. (small cell) LX-1 | 0.2 |
| Brain (thalamus) | 48.6 | Lung ca. (small cell) NCI-H69 | 0.8 |
| Cerebral Cortex | 90.8 | Lung ca. (s.cell var.) SHP-77 | 0.0 |
| Spinal cord | 39.5 | Lung ca. (large cell) NCI-H460 | 0.0 |
| CNS ca. (glio/astro) U87-MG | 1.5 | Lung ca. (non-sm. cell) A549 | 0.9 |
| CNS ca. (glio/astro) U-118-MG | 0.0 | Lung ca. (non-s.cell) NCI-H23 | 0.0 |
| CNS ca. (astro) SW1783 | 1.0 | Lung ca (non-s.cell) HOP-62 | 0.0 |
| CNS ca.* (neuro; met) SK-N-AS | 0.5 | Lung ca. (non-s.cl) NCI-H522 | 1.2 |
| CNS ca. (astro) SF-539 | 0.0 | Lung ca. (squam.) SW 900 | 0.0 |
| CNS ca. (astro) SNB-75 | 0.0 | Lung ca. (squam.) NCI-H596 | 2.7 |
| CNS ca. (glio) SNB-19 | 3.1 | Mammary gland | 1.2 |
| CNS ca. (glio) U251 | 0.0 | Breast ca.* (pl. effusion) MCF-7 | 0.8 |
| CNS ca. (glio) SF-295 | 0.0 | Breast ca.* (pl.ef) | 0.0 |
| | | MDA-MB-231 | |
| Heart (fetal) | 1.1 | Breast ca.* (pl. effusion) T47D | 1.9 |
| Heart | 0.0 | Breast ca. BT-549 | 0.0 |
| Fetal Skeletal | 2.5 | Breast ca. MDA-N | 0.0 |
| Skeletal muscle | 1.1 | Ovary | 0.4 |
| Bone marrow | 36.9 | Ovarian ca. OVCAR-3 | 2.6 |
| Thymus | 21.3 | Ovarian ca. OVCAR-4 | 0.0 |
| Spleen | 100.0 | Ovarian ca. OVCAR-5 | 1.1 |
| Lymph node | 29.3 | Ovarian ca. OVCAR-8 | 0.8 |
| Colorectal | 0.2 | Ovarian ca. IGROV-1 | 0.8 |
| Stomach | 0.6 | Ovarian ca.* (ascites) SK-OV-3 | 0.0 |
| Small intestine | 4.0 | Uterus | 3.0 |
| Colon ca. SW480 | 0.0 | Placenta | 9.2 |
| Colon ca.* (SW480 met)SW620 | 2.4 | Prostate | 2.0 |
| Colon ca. HT29 | 0.8 | Prostate ca.* (bone met) PC-3 | 0.0 |
| Colon ca. HCT-116 | 0.0 | Testis | 6.6 |
| Colon ca. CaCo-2 | 9.3 | Melanoma Hs688(A).T | 0.0 |
| 83219 CC Well to Mod Diff | 3.2 | Melanoma* (met) Hs688(B).T | 0.0 |
| (ODO3866) | | | |
| Colon ca. HCC-2998 | 0.9 | Melanoma UACC-62 | 0.0 |
| Gastric ca.* (liver met) | 0.0 | Melanoma M14 | 0.0 |
| NCI-N87 | | | |
| Bladder | 2.9 | Melanoma LOX IMVI | 0.0 |
| Trachea | 5.3 | Melanoma* (met) SK-MEL-5 | 0.0 |
| Kidney | 0.0 | Adipose | 0.0 |

TABLE 28
Panel 2D

| | Relative | Relative |
|---|---|---|
| | Expression (%) | Expression (%) |
| Tissue Name | 2Dtm2328f_ag1388 | 2dtm4516f_ag2455 |
| Normal Colon GENPAK 061003 | 5.9 | 17.6 |
| 83219 CC Well to Mod Diff (ODO3866) | 10.1 | 9.0 |
| 83220 CC NAT (ODO3866) | 3.2 | 18.8 |
| 83221 CC Gr.2 rectosigmoid (ODO3868) | 4.2 | 4.2 |
| 83222 CC NAT (ODO3868) | 10.9 | 2.7 |
| 83235 CC Mod Diff(ODO3920) | 0.0 | 4.0 |
| 83236 CC NAT (ODO3920) | 5.0 | 3.0 |
| 83237 CC Gr.2 ascend colon (ODO3921) | 9.9 | 7.2 |
| 83238 CC NAT (ODO3921) | 2.4 | 17.0 |
| 83241 CC from Partial Hepatectomy (ODO4309) | 5.2 | 0.0 |
| 83242 Liver NAT (ODO4309) | 0.0 | 0.0 |
| 87472 Colon mets to lung (OD04451-01) | 11.0 | 17.1 |
| 87473 Lung NAT (OD04451-02) | 23.8 | 20.2 |
| Normal Prostate Clontech A + 6546-1 | 6.3 | 4.7 |
| 84140 Prostate Cancer (OD04410) | 11.2 | 12.6 |
| 84141 Prostate NAT (OD04410) | 0.0 | 11.9 |
| 87073 Prostate Cancer (OD04720-01) | 0.0 | 4.4 |
| 87074 Prostate NAT (OD04720-02) | 3.5 | 7.2 |
| Normal Lung GENPAK 061010 | 25.0 | 23.8 |
| 83239 Lung Met to Muscle (ODO4286) | 0.0 | 4.3 |
| 83240 Muscle NAT (ODO4286) | 8.5 | 0.0 |
| 84136 Lung Malignant Cancer (OD03126) | 8.5 | 11.1 |
| 84137 Lung NAT (OD03126) | 0.0 | 15.3 |
| 84871 Lung Cancer (OD04404) | 6.8 | 0.0 |
| 84872 Lung NAT (OD04404) | 15.0 | 18.2 |
| 84875 Lung Cancer (OD04565) | 2.8 | 14.8 |
| 84876 Lung NAT (OD04565) | 5.0 | 13.8 |
| 85950 Lung Cancer (OD04237-01) | 2.4 | 0.0 |
| 85970 Lung NAT (OD04237-02) | 6.4 | 0.0 |
| 83255 Ocular Mel Met to Liver (ODO4310) | 0.0 | 0.0 |
| 83256 Liver NAT (ODO4310) | 4.8 | 0.0 |
| 84139 Melanoma Mets to Lung (OD04321) | 2.7 | 0.0 |
| 84138 Lung NAT (OD04321) | 13.5 | 31.4 |
| Normal Kidney GENPAK 061008 | 0.5 | 4.7 |
| 83786 Kidney Ca, Nuclear grade 2 (OD04338) | 9.8 | 7.5 |
| 83787 Kidney NAT (OD04338) | 1.6 | 2.1 |
| 83788 Kidney Ca Nuclear grade 1/2 (OD04339) | 2.3 | 10.2 |
| 83789 Kidney NAT (OD04339) | 0.0 | 0.0 |
| 83790 Kidney Ca, Clear cell type (OD04340) | 12.0 | 10.4 |
| 83791 Kidney NAT (OD04340) | 2.9 | 10.0 |
| 83792 Kidney Ca, Nuclear grade 3 (OD04348) | 5.6 | 2.5 |
| 83793 Kidney NAT (OD04348) | 5.5 | 1.4 |
| 87474 Kidney Cancer (OD04622-01) | 33.0 | 21.9 |
| 87475 Kidney NAT (OD04622-03) | 5.0 | 5.5 |
| 85973 Kidney Cancer (OD04450-01) | 0.0 | 0.0 |
| 85974 Kidney NAT (OD04450-03) | 0.0 | 0.0 |
| Kidney Cancer Clontech 8120607 | 0.0 | 9.1 |
| Kidney NAT Clontech 8120608 | 5.3 | 2.6 |
| Kidney Cancer Clontech 8120613 | 0.0 | 0.0 |
| Kidney NAT Clontech 8120614 | 0.0 | 0.0 |
| Kidney Cancer Clontech 9010320 | 24.7 | 32.5 |
| Kidney NAT Clontech 9010321 | 8.4 | 0.0 |
| Normal Uterus GENPAK 061018 | 3.7 | 2.0 |
| Uterus Cancer GENPAK 064011 | 5.7 | 2.3 |
| Normal Thyroid Clontech A + 6570-1 | 3.2 | 4.4 |
| Thyroid Cancer GENPAK 064010 | 3.1 | 2.4 |
| Thyroid Cancer INVITROGEN A302152 | 4.4 | 9.3 |
| Thyroid NAT INVITROGEN A302153 | 0.0 | 11.0 |
| Normal Breast GENPAK 061019 | 34.9 | 23.3 |
| 84877 Breast Cancer (OD04566) | 0.9 | 11.1 |
| 85975 Breast Cancer (OD04590-01) | 33.9 | 39.0 |
| 85976 Breast Cancer Mets (OD04590-03) | 92.0 | 76.8 |
| 87070 Breast Cancer Metastasis (OD04655-05) | 100.0 | 100.0 |
| GENPAK Breast Cancer 064006 | 0.0 | 12.2 |
| Breast Cancer Res. Gen. 1024 | 5.5 | 13.7 |
| Breast Cancer Clontech 9100266 | 0.9 | 2.6 |
| Breast NAT Clontech 9100265 | 0.0 | 5.2 |
| Breast Cancer INVITROGEN A209073 | 6.0 | 4.2 |
| Breast NAT INVITROGEN A2090734 | 4.6 | 12.6 |
| Normal Liver GENPAK 061009 | 2.7 | 0.0 |
| Liver Cancer GENPAK 064003 | 3.3 | 1.3 |
| Liver Cancer Research Genetics RNA 1025 | 9.0 | 2.3 |
| Liver Cancer Research Genetics RNA 1026 | 5.6 | 1.6 |
| Paired Liver Cancer Tissue | 6.0 | 3.6 |
| Research Genetics RNA 6004-T | | |
| Paired Liver Tissue Research | 0.0 | 2.6 |
| Genetics RNA 6004-N | | |
| Paired Liver Cancer Tissue Research | 3.2 | 2.1 |
| Genetics RNA 6005-T | | |
| Paired Liver Tissue Research | 8.1 | 0.0 |
| Genetics RNA 6005-N | | |
| Normal Bladder GENPAK 061001 | 11.8 | 17.9 |
| Bladder Cancer Research Genetics RNA 1023 | 7.2 | 1.9 |
| Bladder Cancer INVITROGEN A302173 | 6.0 | 2.7 |
| 87071 Bladder Cancer (OD04718-01) | 1.5 | 2.6 |
| 87072 Bladder Normal Adjacent (OD04718-03) | 11.3 | 1.9 |
| Normal Ovary Res. Gen. | 0.3 | 2.1 |
| Ovarian Cancer GENPAK 064008 | 8.4 | 1.6 |
| 87492 Ovary Cancer (OD04768-07) | 0.0 | 2.7 |
| 87493 Ovary NAT (OD04768-08) | 0.0 | 0.0 |
| Normal Stomach GENPAK 061017 | 6.6 | 13.2 |
| Gastric Cancer Clontech 9060358 | 2.1 | 4.8 |
| NAT Stomach Clontech 9060359 | 6.0 | 7.1 |
| Gastric Cancer Clontech 9060395 | 11.0 | 7.5 |
| NAT Stomach Clontech 9060394 | 3.6 | 9.9 |
| Gastric Cancer Clontech 9060397 | 3.1 | 0.0 |
| NAT Stomach Clontech 9060396 | 0.0 | 4.3 |
| Gastric Cancer GENPAK 064005 | 0.0 | 6.8 |

TABLE 29
Panels 4D/4R

| | | | Relative |
|---|---|---|---|
| | Relative Expression (%) | | Expression (%) |
| | 4Dtm1781f_{—} | 4rtm1790f_{—} | 4Dx4tm4260f_{—} |
| Tissue Name | ag1388 | ag1388 | ag2455_a1 |
| 93768_Secondary Th1_anti-CD28/anti-CD3 | 2.6 | 2.9 | 5.3 |
| 93769_Secondary Th2_anti-CD28/anti-CD3 | 4.9 | 3.7 | 6.5 |
| 93770_Secondary Tr1_anti-CD28/anti-CD3 | 9.9 | 3.4 | 5.1 |
| 93573_Secondary Th1_resting day 4-6 in IL-2 | 12.0 | 12.2 | 6.8 |
| 93572_Secondary Th2_resting day 4-6 in IL-2 | 12.1 | 16.4 | 14.9 |
| 93571_Secondary Tr1_resting day 4-6 in IL-2 | 11.5 | 16.2 | 25.2 |
| 93568_primary Th1_anti-CD28/anti-CD3 | 5.0 | 6.2 | 1.8 |
| 93569_primary Th2_anti-CD28/anti-CD3 | 4.6 | 5.5 | 1.0 |
| 93570_primary Tr1_anti-CD28/anti-CD3 | 5.8 | 4.0 | 1.4 |
| 93565_primary Th1_resting dy 4-6 in IL-2 | 31.2 | 100.0 | 23.4 |
| 93566_primary Th2_resting dy 4-6 in IL-2 | 36.1 | 55.1 | 27.2 |
| 93567_primary Tr1_resting dy 4-6 in IL-2 | 22.1 | 4.9 | 28.0 |
| 93351_CD45RA CD4 lymphocyte_{—} | 0.0 | 0.9 | 2.4 |
| anti-CD28/anti-CD3 | | | |
| 93352_CD45RO CD4 lymphocyte_{—} | 3.6 | 4.0 | 2.2 |
| anti-CD28/anti-CD3 | | | |
| 93251_CD8 Lymphocytes_{—} | 3.6 | 2.4 | 2.5 |
| anti-CD28/anti-CD3 | | | |
| 93353_chronic CD8 Lymphocytes | 3.7 | 3.0 | 0.0 |
| 2ry_resting dy 4-6 in IL-2 | | | |
| 93574_chronic CD8 Lymphocytes 2ry_activated | 3.7 | 3.6 | 3.4 |
| CD3/CD28 | | | |
| 93354_CD4_none | 7.3 | 11.6 | 17.4 |
| 93252_Secondary Th1/Th2/Tr1_{—} | 39.2 | 43.2 | 18.5 |
| anti-CD95 CH11 | | | |
| 93103_LAK cells_resting | 6.4 | 4.5 | 5.1 |
| 93788_LAK cells_IL-2 | 9.4 | 8.2 | 5.3 |
| 93787_LAK cells_IL-2 + IL-12 | 2.5 | 7.5 | 2.7 |
| 93789_LAK cells_IL-2 + IFN gamma | 3.9 | 13.7 | 3.7 |
| 93790_LAK cells_IL-2 + IL-18 | 1.5 | 1.3 | 3.2 |
| 93104_LAK cells_PMA/ionomycin and IL-18 | 0.0 | 0.4 | 1.0 |
| 93578_NK Cells IL-2_resting | 10.0 | 9.2 | 10.6 |
| 93109_Mixed Lymphocyte Reaction_Two Way | 3.1 | 6.9 | 8.8 |
| MLR | | | |
| 93110_Mixed Lymphocyte Reaction_Two Way | 0.0 | 1.4 | 4.0 |
| MLR | | | |
| 93111_Mixed Lymphocyte Reaction_Two Way | 6.7 | 6.0 | 6.5 |
| MLR | | | |
| 93112_Mononuclear Cells (PBMCs)_resting | 9.7 | 12.5 | 5.9 |
| 93113_Mononuclear Cells (PBMCs)_PWM | 5.0 | 12.2 | 2.7 |
| 93114_Mononuclear Cells (PBMCs)_PHA-L | 5.6 | 5.1 | 4.7 |
| 93249_Ramos (B cell)_none | 6.8 | 5.7 | 4.7 |
| 93250_Ramos (B cell)_ionomycin | 4.1 | 48.3 | 9.4 |
| 93349_B lymphocytes_PWM | 7.1 | 17.7 | 10.3 |
| 93350_B lymphoytes_{—} | 21.8 | 2.7 | 36.9 |
| CD40L and IL-4 | | | |
| 92665_EOL-1 (Eosinophil)_{—} | 100.0 | 65.1 | 100.0 |
| dbcAMP differentiated | | | |
| 93248_EOL-1 | 17.6 | 35.4 | 37.9 |
| (Eosinophil)_dbcAMP/PMAionomycin | | | |
| 93356_Dendritic Cells_none | 1.4 | 2.0 | 0.0 |
| 93355_Dendritic Cells_LPS 100 ng/ml | 0.0 | 0.0 | 2.5 |
| 93775_Dendritic Cells_anti-CD40 | 0.0 | 0.7 | 0.7 |
| 93774_Monocytes_resting | 10.5 | 23.2 | 18.7 |
| 93776_Monocytes_LPS 50 ng/ml | 2.6 | 6.2 | 3.0 |
| 93581_Macrophages_resting | 2.8 | 5.1 | 5.5 |
| 93582_Macrophages_LPS 100 ng/ml | 0.0 | 1.1 | 1.6 |
| 93098_HUVEC (Endothelial)_none | 0.0 | 0.1 | 0.0 |
| 93099_HUVEC (Endothelial)_starved | 0.0 | 0.0 | 0.0 |
| 93100_HUVEC (Endothelial)_IL-1b | 0.0 | 0.2 | 0.0 |
| 93779_HUVEC (Endothelial)_IFN gamma | 0.0 | 0.0 | 0.0 |
| 93102_HUVEC (Endothelial)_TNF alpha + IFN | 0.0 | 0.0 | 0.0 |
| gamma | | | |
| 93101_HUVEC (Endothelial)_TNF alpha + IL4 | 0.0 | 0.1 | 0.0 |
| 93781_HUVEC (Endothelial)_IL-11 | 0.0 | 0.0 | 0.0 |
| 93583_Lung Microvascular Endothelial | 0.0 | 0.0 | 0.0 |
| Cells_none | | | |
| 93584_Lung Microvascular | 0.0 | 0.4 | 0.0 |
| Endothelial Cells_TNFa | | | |
| (4 ng/ml) and IL1b (1 ng/ml) | | | |
| 92662_Microvascular Dermal endothelium_none | 0.0 | 0.4 | 0.0 |
| 92663_Microsvasular Dermal | 0.0 | 0.3 | 0.0 |
| endothelium_TNFa (4 ng/ml) and IL1b (1 ng/ml) | | | |
| 93773_Bronchial epithelium_{—} | 1.5 | 0.0 | 0.0 |
| TNFa (4 ng/ml) and IL1b (1 ng/ml)** | | | |
| 93347_Small Airway Epithelium_none | 0.0 | 0.0 | 0.0 |
| 93348_Small Airway Epithelium_{—} | 0.0 | 0.2 | 0.7 |
| TNFa (4 ng/ml) and IL1b (1 ng/ml) | | | |
| 92668_Coronery Artery SMC_resting | 1.3 | 0.0 | 0.0 |
| 92669_Coronery Artery SMC_TNFa | 0.0 | 0.0 | 0.0 |
| (4 ng/ml) and IL1b (1 ng/ml) | | | |
| 93107_astrocytes_resting | 0.0 | 0.5 | 0.0 |
| 93108_astrocytes_TNFa (4 ng/ml) and IL1b | 0.0 | 0.0 | 0.0 |
| (1 ng/ml) | | | |
| 92666_KU-812 (Basophil)_resting | 1.9 | 5.6 | 3.5 |
| 92667_KU-812 (Basophil)_PMA/ionoycin | 2.4 | 6.7 | 3.9 |
| 93579_CCD1106 (Keratinocytes)_none | 0.0 | 0.0 | 0.0 |
| 93580_CCD1106 (Keratinocytes)_{—} | 0.0 | 0.4 | 0.0 |
| TNFa and IFNg** | | | |
| 93791_Liver Cirrhosis | 10.7 | 1.8 | 7.3 |
| 93792_Lupus Kidney | 2.5 | 2.6 | 1.5 |
| 93577_NCI-H292 | 0.0 | 4.5 | 3.8 |
| 93358_NCI-H292_IL-4 | 0.0 | 2.3 | 0.0 |
| 93360_NCI-H292_IL-9 | 1.3 | 0.4 | 0.5 |
| 93359_NCI-H292_IL-13 | 0.0 | 0.4 | 0.0 |
| 93357_NCI-H292_IFN gamma | 0.0 | 0.5 | 0.0 |
| 93777_HPAEC_- | 0.0 | 0.0 | 0.0 |
| 93778_HPAEC_IL-1 beta/TNA alpha | 0.0 | 0.1 | 0.4 |
| 93254_Normal Human Lung Fibroblast_none | 0.0 | 0.8 | 0.0 |
| 93253_Normal Human Lung Fibroblast_{—} | 0.0 | 0.6 | 0.0 |
| TNFa (4 ng/ml) and IL-1b (1 ng/ml) | | | |
| 93257_Normal Human Lung Fibroblast_IL-4 | 0.0 | 0.0 | 0.0 |
| 93256_Normal Human Lung Fibroblast_IL-9 | 0.0 | 0.0 | 0.0 |
| 93255_Normal Human Lung Fibroblast_IL-13 | 0.0 | 0.0 | 0.0 |
| 93258_Normal Human Lung Fibroblast_{—} | 0.0 | 0.0 | 0.0 |
| IFN gamma | | | |
| 93106_Dermal Fibroblasts CCD1070_{—} | 0.0 | 0.2 | 0.0 |
| resting | | | |
| 93361_Dermal Fibroblasts CCD1070_{—} | 8.9 | 19.2 | 9.4 |
| TNF alpha 4 ng/ml | | | |
| 93105_Dermal Fibroblasts CCD1070_{—} | 0.0 | 0.0 | 0.5 |
| IL-1 beta 1 ng/ml | | | |
| 93772_dermal fibroblast_IFN gamma | 0.0 | 0.0 | 0.0 |
| 93771_dermal fibroblast_IL-4 | 0.0 | 0.0 | 0.0 |
| 93259_IBD Colitis 1** | 2.9 | 0.2 | 0.9 |
| 93260_IBD Colitis 2 | 1.5 | 1.1 | 2.4 |
| 93261_IBD Crohns | 1.4 | 0.5 | 0.0 |
| 735010_Colon_normal | 34.9 | 5.5 | 31.0 |
| 735019_Lung_none | 11.8 | 2.7 | 11.6 |
| 64028-1_Thymus_none | 1.5 | 0.4 | 0.9 |
| 64030-1_Kidney_none | 4.5 | 6.7 | 11.8 |

TABLE 30
Panel CNSD.01

| | Relative | | Relative |
|---|---|---|---|
| | Expression (%) | | Expression (%) |
| | cns1x4tm6186f_{—} | | cns1x4tm6186f_{—} |
| Tissue Name | ag2455_a2 | Tissue Name | ag2455_a2 |
| 102633_BA4 Control | 9.1 | 102605_BA17 PSP | 1.3 |
| 102641_BA4 Control2 | 59.7 | 102612_BA17 PSP2 | 8.6 |
| 102625_BA4 Alzheimer's2 | 14.7 | 102637_Sub Nigra Control | 31.8 |
| 102649_BA4 Parkinson's | 20.1 | 102645_Sub Nigra Control2 | 35.7 |
| 102656_BA4 Parkinson's2 | 51.0 | 102629_Sub Nigra Alzheimer's2 | 15.7 |
| 102664_BA4 Huntington's | 5.6 | 102660_Sub Nigra Parkinson's2 | 29.9 |
| 102671_BA4 Huntington's2 | 29.6 | 102667_Sub Nigra Huntington's | 50.1 |
| 102603_BA4 PSP | 9.6 | 102674_Sub Nigra Huntington's2 | 9.8 |
| 102610_BA4 PSP2 | 22.2 | 102614_Sub Nigra PSP2 | 2.0 |
| 102588_BA4 Depression | 0.9 | 102592_Sub Nigra Depression | 0.0 |
| 102596_BA4 Depression2 | 8.8 | 102599_Sub Nigra Depression2 | 0.0 |
| 102634_BA7 Control | 13.6 | 102636_Glob Palladus Control | 43.8 |
| 102642_BA7 Control2 | 31.4 | 102644_Glob Palladus Control2 | 100.0 |
| 102626_BA7 Alzheimer's2 | 0.0 | 102620_Glob Palladus Alzheimer's | 18.3 |
| 102650_BA7 Parkinson's | 23.4 | 102628_Glob Palladus | 15.2 |
| | | Alzheimer's2 | |
| 102657_BA7 Parkinson's2 | 27.4 | 102652_Glob Palladus Parkinson's | 24.9 |
| 102665_BA7 Huntington's | 21.7 | 102659_Glob Palladus | 66.9 |
| | | Parkinson's2 | |
| 102672_BA7 Huntington's2 | 36.8 | 102606_Glob Palladus PSP | 45.4 |
| 102604_BA7 PSP | 7.7 | 102613_Glob Palladus PSP2 | 43.1 |
| 102611_BA7 PSP2 | 0.0 | 102591_Glob Palladus Depression | 12.9 |
| 102589_BA7 Depression | 9.0 | 102638_Temp Pole Control | 23.1 |
| 102632_BA9 Control | 3.7 | 102646_Temp Pole Control2 | 67.9 |
| 102640_BA9 Control2 | 30.4 | 102622_Temp Pole Alzheimer's | 2.5 |
| 102617_BA9 Alzheimer's | 0.0 | 102630_Temp Pole Alzheimer's2 | 6.7 |
| 102624_BA9 Alzheimer's2 | 1.7 | 102653_Temp Pole Parkinson's | 39.7 |
| 102648_BA9 Parkinson's | 6.8 | 102661_Temp Pole Parkinson's2 | 12.8 |
| 102655_BA9 Parkinson's2 | 15.7 | 102668_Temp Pole Huntington's | 26.1 |
| 102663_BA9 Huntington's | 21.7 | 102607_Temp Pole PSP | 0.0 |
| 102670_BA9 Huntington's2 | 1.1 | 102615_Temp Pole PSP2 | 0.0 |
| 102602_BA9 PSP | 3.6 | 102600_Temp Pole Depression2 | 4.8 |
| 102609_BA9 PSP2 | 6.2 | 102639_Cing Gyr Control | 36.1 |
| 102587_BA9 Depression | 8.5 | 102647_Cing Gyr Control2 | 28.9 |
| 102595_BA9 Depression2 | 0.0 | 102623_Cing Gyr Alzheimer's | 7.0 |
| 102635_BA17 Control | 12.7 | 102631_Cing Gyr Alzheimer's2 | 0.0 |
| 102643_BA17 Control2 | 36.0 | 102654_Cing Gyr Parkinson's | 17.7 |
| 102627_BA17 Alzheimer's2 | 5.3 | 102662_Cing Gyr Parkinson's2 | 14.1 |
| 102651_BA17 Parkinson's | 23.5 | 102669_Cing Gyr Huntington's | 52.1 |
| 102658_BA17 Parkinson's2 | 18.3 | 102676_Cing Gyr Huntington's2 | 8.5 |
| 102666_BA17 Huntington's | 24.9 | 102608_Cing Gyr PSP | 0.0 |
| 102673_BA17 Huntington's2 | 6.8 | 102616_Cing Gyr PSP2 | 0.6 |
| 102590_BA17 Depression | 3.7 | 102594_Cing Gyr Depression | 5.0 |
| 102597_BA17 Depression2 | 6.6 | 102601_Cing Gyr Depression2 | 3.4 |

Panel 1.2 Summary: Ag1388 Expression of the NOV3 gene in the samples on this panel seems to be restricted, in large part, to normal tissues. The NOV3 gene is most highly expressed in a sample derived from cerebellum (CT=26). Expression of this gene is also prominent in stomach. Based upon this pattern of expression, the expression of this gene might be of use as a marker of cerebellar or stomach tissue.

Among CNS samples, the NOV3 gene is expressed in cerebellum, amygdala, hippocampus, thalamus, cerebral cortex and spinal cord. This result is consistent with what is observed in Panel 1.3D; please see below for summary of potential implications of the expression of this gene in the central nervous system.

The NOV3 gene encodes a type 1 membrane protein with several leucine-rich-repeat domains, indicating that this gene product may be involved in extracellular signalling and/or interactions with the extracellular matrix. Among metabolically relevant tissues, this gene is expressed at low but significant levels in the adrenal gland, thyroid, heart and liver. As a potential extracellular signalling molecule, the NOV3 gene product may serve as an antibody target for diseases involving any or all of these tissues.

Panel 1.3D Summary: Ag2455 Expression of the NOV3 gene in this panel is largely restricted to normal brain and normal lymphoid tissues. Highest expression of this gene is detected in spleen (CT=30), with lower but significant expression in lymph node, bone marrow and thymus. Thus, the expression of this gene might be useful as a marker of lymphoid tissue.

Moderate and roughly equivalent expression is also detected in several regions of the CNS including amygdala, cerebellum, substantia nigra, hippocampus, thalamus, cerebral cortex and spinal cord. In Drosophilia, the LRR region of axon guidance proteins has been shown to be critical for function (especially in axon repulsion) (ref. 1). Since the NOV3 gene encodes a leucine-rich-repeat protein that is expressed across all brain regions, it is an excellent candidate neuronal guidance protein for axons, dendrites and/or growth cones in general. Therefore, therapeutic modulation of the levels of this protein, or possible signaling via this protein, may be of utility in enhancing/directing compensatory synaptogenesis and fiber growth in the CNS in response to neuronal death (stroke, head trauma), axon lesion (spinal cord injury), or neurodegeneration (Alzheimer's, Parkinson's, Huntington's, vascular dementia or any neurodegenerative disease).

Panel 2D Summary: Ag1388/Ag2455 Results from two experiments using different probe/primer sets are in good agreement. Strikingly, expression of the NOV3 gene is highest in two metastatic breast cancer samples (CT=31-32), and is also detectable in several other breast cancer samples. In addition, there appears to be a moderate association with overexpression of the NOV3 gene in kidney cancers when compared to their normal adjacent tissues, as 6 of 9 pairs show this pattern of expression. Thus, expression of this gene could be used as a marker for the detection of breast or kidney cancer. In addition, therapeutic down modulation of the NOV3 gene product, through the use of antibodies or small molecule drugs, may be useful for the treatment of breast or kidney cancer.

Panel 4D/4R Summary: Ag1388/Ag2455 Significant expression of the NOV3 gene is detected in bone marrow, spleen, and lymph node, as well as in the thymus in one experiment. These results are consistent with what is observed in Panel 1.3D. In addition, differential NOV3 gene expression is observed in the eosinophil cell line EOL-1 under resting conditions over that in EOL-1 cells stimulated by phorbol ester and ionomycin. Furthermore, unstimulated T lymphocytes (Th1, Th2, and Tr1) expressed this gene at higher levels than anti-CD28+anti-CD3-stimulated T cells. Thus, the NOV3 gene may be involved in both eosinophil and T lymphocyte function. Antibodies raised against the NOV3 protein that stimulate its activity may be useful in reduction of eosinophil activation and may therefore be useful therapeutic antibodies for asthma and allergy, and also as anti-inflammatory therapeutics for T cell-mediated autoimmune and inflammatory diseases. Furthermore, the isolated extracellular domain of the NOV3 protein may likewise function as a protein therapeutic in the treatment of asthma, emphysema, and allergy, as well as in other autoimmune and inflammatory diseases such as rheumatoid arthritis, inflammatory bowel disease, and psoriasis.

Panel CNSD.01 Summary: Ag2455 Among the samples on this panel, the NOV3 gene is most highly expressed in the globus palladus, a region of the basal ganglia involved in the control of movement; various inputs to the globus palladus are lost in Parkinson's disease and Huntington's disease. Since there is evidence that leucine-rich repeat proteins are critical in axonal guidance, the protein encoded by the NOV3 gene may be important in the treatment of Parkinson's and/or Huntington's disease by stimulating neuroregeneration and/or stem cell implantation for the establishment of connectivity. Likewise modulation of the activity of this protein may serve to slow or stop neurodegeneration in these diseases. (Battye et al., Repellent signaling by Slit requires the leucine-rich repeats. J. Neurosci. 21: 4290-4298, 2001.)

NOV4: Cathepsin-L Precursor-like

Expression of the NOV4 gene (GMba39917_A) was assessed using the primer-probe sets Ag2453 described in Table 31.

TABLE 31

Probe Name Ag2453

| | | | | | |
|---|---|---|---|---|---|
| | | | | Start | |
| Primers | Sequences | TM | Length | Position | SEQ ID NO: |

| | | | | | |
|---|---|---|---|---|---|
| Forward | 5′-CTCTGGAAGGGCAGATGTTT-3′ | 59.3 | 20 | 473 | 161 |
| Probe | FAM-5′- | 66.9 | 28 | 493 | 162 |
| | TGGAAAACAGGCAAACTTATCTCACTGA- | | | | |
| | 3′-TAMRA | | | | |
| Reverse | 5′-CCAGAGCAGTCTACCAGATTGA-3′ | 59.5 | 22 | 527 | 163 |

Expression of this gene in panels 1.3D, 2D, 4D, and Cns_Neurodegeneration_V1.0 was low/undetectable (Ct values>35) in all samples (data not shown).

NOV5: Fatty Acid-Binding Protein-like

Expression of the NOV5 gene (GMba38118_A) was assessed using the primer-probe set Ag2456, described in Table 32. Results of the RTQ-PCR runs are shown in Tables 33, 34, 35, 36, and 37.

TABLE 32

Probe Name Ag2456

| | | | | | |
|---|---|---|---|---|---|
| | | | | Start | |
| Primers | Sequences | TM | Length | Position | SEQ ID NO: |

| | | | | | |
|---|---|---|---|---|---|
| Forward | 5′-AGTGGTGGAGTGTGTCATGAA-3′ | 59 | 21 | 404 | 164 |
| Probe | TET-5′- | 64.5 | 27 | 425 | 165 |
| | CAATGTCACCTGTACTCGGATCTATGA-3′- | | | | |
| | TAMRA | | | | |
| Reverse | 5′-CTGTCCAAAGTGATGATGGAA-3′ | 58.6 | 21 | 468 | 166 |

TABLE 33
Panel 1.3D

| | Relative | | Relative |
|---|---|---|---|
| | Expression (%) | | Expression (%) |
| | 1.3dtm3778t_{—} | | 1.3dtm3778t_{—} |
| Tissue Name | ag2456 | Tissue Name | ag2456 |
| Liver adenocarcinoma | 2.7 | Kidney (fetal) | 3.0 |
| Pancreas | 4.2 | Renal ca. 786-0 | 3.6 |
| Pancreatic ca. CAPAN 2 | 1.0 | Renal ca. A498 | 20.6 |
| Adrenal gland | 2.3 | Renal ca. RXF 393 | 2.0 |
| Thyroid | 4.0 | Renal ca. ACHN | 0.8 |
| Salivary gland | 2.3 | Renal ca. UO-31 | 12.4 |
| Pituitary gland | 3.3 | Renal ca. TK-10 | 1.3 |
| Brain (fetal) | 15.6 | Liver | 0.9 |
| Brain (whole) | 5.4 | Liver (fetal) | 6.1 |
| Brain (amygdala) | 14.6 | Liver ca. (hepatoblast) HepG2 | 2.9 |
| Brain (cerebellum) | 4.6 | Lung | 28.9 |
| Brain (hippocampus) | 71.2 | Lung (fetal) | 7.8 |
| Brain (substantia nigra) | 7.9 | Lung ca. (small cell) LX-1 | 4.2 |
| Brain (thalamus) | 11.7 | Lung ca. (small cell) NCI-H69 | 14.3 |
| Cerebral Cortex | 12.4 | Lung ca. (s.cell var.) SHP-77 | 20.9 |
| Spinal cord | 13.7 | Lung ca. (large cell)NCI-H460 | 3.1 |
| CNS ca. (glio/astro) U87-MG | 3.6 | Lung ca. (non-sm. cell) A549 | 3.3 |
| CNS ca. (glio/astro) U-118-MG | 1.7 | Lung ca. (non-s.cell) NCI-H23 | 7.7 |
| CNS ca. (astro) SW1783 | 0.7 | Lung ca (non-s.cell) HOP-62 | 1.9 |
| CNS ca.* (neuro; met) SK-N-AS | 25.3 | Lung ca. (non-s.cl) NCI-H522 | 0.4 |
| CNS ca. (astro) SF-539 | 1.4 | Lung ca. (squam.) SW 900 | 0.5 |
| CNS ca. (astro) SNB-75 | 1.2 | Lung ca. (squam.) NCI-H596 | 9.7 |
| CNS ca. (glio) SNB-19 | 0.7 | Mammary gland | 15.9 |
| CNS ca. (glio) U251 | 0.4 | Breast ca.* (pl. effusion) MCF-7 | 1.2 |
| CNS ca. (glio) SF-295 | 0.4 | Breast ca.* (pl.ef) MDA-MB-231 | 20.9 |
| Heart (fetal) | 7.9 | Breast ca.* (pl. effusion) T47D | 0.5 |
| Heart | 12.5 | Breast ca. BT-549 | 32.3 |
| Fetal Skeletal | 14.3 | Breast ca. MDA-N | 1.3 |
| Skeletal muscle | 4.3 | Ovary | 1.2 |
| Bone marrow | 6.9 | Ovarian ca. OVCAR-3 | 2.1 |
| Thymus | 4.2 | Ovarian ca. OVCAR-4 | 0.1 |
| Spleen | 17.1 | Ovarian ca. OVCAR-5 | 4.1 |
| Lymph node | 6.6 | Ovarian ca. OVCAR-8 | 4.7 |
| Colorectal | 7.7 | Ovarian ca. IGROV-1 | 2.5 |
| Stomach | 16.0 | Ovarian ca.* (ascites) SK-OV-3 | 9.5 |
| Small intestine | 23.0 | Uterus | 3.4 |
| Colon ca. SW480 | 14.6 | Placenta | 21.8 |
| Colon ca.* (SW480 met) SW620 | 10.3 | Prostate | 2.6 |
| Colon ca. HT29 | 6.0 | Prostate ca.* (bone met) PC-3 | 6.7 |
| Colon ca. HCT-116 | 23.2 | Testis | 7.4 |
| Colon ca. CaCo-2 | 13.1 | Melanoma Hs688(A).T | 0.7 |
| 83219 CC Well to Mod Diff | 16.5 | Melanoma* (met) Hs688(B).T | 0.6 |
| (ODO3866) | | | |
| Colon ca. HCC-2998 | 25.3 | Melanoma UACC-62 | 2.5 |
| Gastric ca.* (liver met) | 17.0 | Melanoma M14 | 2.5 |
| NCI-N87 | | | |
| Bladder | 3.7 | Melanoma LOX IMVI | 16.8 |
| Trachea | 20.0 | Melanoma* (met) SK-MEL-5 | 100.0 |
| Kidney | 0.8 | Adipose | 13.4 |

TABLE 34
Panel 2D

| | Relative | | Relative |
|---|---|---|---|
| | Expression (%) | | Expression (%) |
| | 2dtm3779t_{—} | | 2dtm3779t_{—} |
| Tissue Name | ag2456 | Tissue Name | ag2456 |
| Normal Colon GENPAK 061003 | 13.9 | Kidney NAT Clontech 8120608 | 0.1 |
| 83219 CC Well to Mod Diff | 3.3 | Kidney Cancer Clontech 8120613 | 0.2 |
| (ODO3866) | | | |
| 83220 CC NAT (ODO3866) | 2.7 | Kidney NAT Clontech 8120614 | 0.0 |
| 83221 CC Gr.2 rectosigmoid | 1.7 | Kidney Cancer Clontech 9010320 | 1.8 |
| (ODO3868) | | | |
| 83222 CC NAT (ODO3868) | 0.6 | Kidney NAT Clontech 9010321 | 0.2 |
| 83235 CC Mod Diff (ODO3920) | 4.2 | Normal Uterus GENPAK 061018 | 0.5 |
| 83236 CC NAT (ODO3920) | 1.7 | Uterus Cancer GENPAK 064011 | 1.6 |
| 83237 CC Gr.2 ascend colon | 10.2 | Normal Thyroid Clontech A + | 1.0 |
| (ODO3921) | | 6570-1 | |
| 83238 CC NAT (ODO3921) | 3.1 | Thyroid Cancer GENPAK 064010 | 0.6 |
| 83241 CC from Partial | 2.6 | Thyroid Cancer INVITROGEN | 0.6 |
| Hepatectomy (ODO4309) | | A302152 | |
| 83242 Liver NAT (ODO4309) | 0.3 | Thyroid NAT INVITROGEN | 1.4 |
| | | A302153 | |
| 87472 Colon mets to lung | 1.4 | Normal Breast GENPAK 061019 | 2.1 |
| (OD04451-01) | | | |
| 87473 Lung NAT (0D04451-02) | 2.5 | 84877 Breast Cancer (OD04566) | 0.7 |
| Normal Prostate Clontech A + | 0.5 | 85975 Breast Cancer | 2.0 |
| 6546-1 | | (OD04590-01) | |
| 84140 Prostate Cancer (OD04410) | 2.0 | 85976 Breast Cancer Mets | 3.1 |
| | | (OD04590-03) | |
| 84141 Prostate NAT (OD04410) | 4.0 | 87070 Breast Cancer Metastasis | 1.1 |
| | | (OD04655-05) | |
| 87073 Prostate Cancer | 1.1 | GENPAK Breast Cancer 064006 | 1.8 |
| (OD04720-01) | | | |
| 87074 Prostate NAT | 1.5 | Breast Cancer Res. Gen. 1024 | 1.0 |
| (OD04720-02) | | | |
| Normal Lung GENPAK 061010 | 11.3 | Breast Cancer Clontech 9100266 | 0.8 |
| 83239 Lung Met to Muscle | 1.4 | Breast NAT Clontech 9100265 | 0.6 |
| (ODO4286) | | | |
| 83240 Muscle NAT (ODO4286) | 1.2 | Breast Cancer INVITROGEN | 1.5 |
| | | A209073 | |
| 84136 Lung Malignant Cancer | 5.8 | Breast NAT INVITROGEN | 0.7 |
| (OD03126) | | A2090734 | |
| 84137 Lung NAT (OD03126) | 20.7 | Normal Liver GENPAK 061009 | 0.0 |
| 84871 Lung Cancer (OD04404) | 100.0 | Liver Cancer GENPAK 064003 | 0.2 |
| 84872 Lung NAT (OD04404) | 3.3 | Liver Cancer Research Genetics | 0.1 |
| | | RNA 1025 | |
| 84875 Lung Cancer (OD04565) | 36.1 | Liver Cancer Research Genetics | 0.2 |
| | | RNA 1026 | |
| 84876 Lung NAT (OD04565) | 2.9 | Paired Liver Cancer Tissue | 0.2 |
| | | Research Genetics RNA 6004-T | |
| 85950 Lung Cancer (OD04237-01) | 5.7 | Paired Liver Tissue Research | 0.6 |
| | | Genetics RNA 6004-N | |
| 85970 Lung NAT (OD04237-02) | 4.2 | Paired Liver Cancer Tissue | 0.2 |
| | | Research Genetics RNA 6005-T | |
| 83255 Ocular Mel Met to Liver | 1.3 | Paired Liver Tissue Research | 0.0 |
| (OD04310) | | Genetics RNA 6005-N | |
| 83256 Liver NAT (ODO4310) | 0.4 | Normal Bladder GENPAK 061001 | 2.6 |
| 84139 Melanoma Mets to Lung | 3.2 | Bladder Cancer Research Genetics | 0.5 |
| (OD04321) | | RNA 1023 | |
| 84138 Lung NAT (OD04321) | 8.7 | Bladder Cancer INVITROGEN | 12.9 |
| | | A302173 | |
| Normal Kidney GENPAK 061008 | 1.0 | 87071 Bladder Cancer | 4.0 |
| | | (OD04718-01) | |
| 83786 Kidney Ca, Nuclear grade 2 | 1.2 | 87072 Bladder Normal Adjacent | 1.5 |
| (OD04338) | | (OD04718-03) | |
| 83787 Kidney NAT (OD04338) | 0.7 | Normal Ovary Res. Gen. | 0.2 |
| 83788 Kidney Ca Nuclear grade | 1.3 | Ovarian Cancer GENPAK 064008 | 5.8 |
| 1/2 (OD04339) | | | |
| 83789 Kidney NAT (OD04339) | 1.0 | 87492 Ovary Cancer | 3.1 |
| | | (OD04768-07) | |
| 83790 Kidney Ca, Clear cell type | 1.7 | 87493 Ovary NAT (OD04768-08) | 1.1 |
| (OD04340) | | | |
| 83791 Kidney NAT (OD04340) | 0.6 | Normal Stomach GENPAK | 1.5 |
| | | 061017 | |
| 83792 Kidney Ca, Nuclear grade 3 | 2.5 | Gastric Cancer Clontech 9060358 | 0.7 |
| (OD04348) | | | |
| 83793 Kidney NAT (OD04348) | 0.4 | NAT Stomach Clontech 9060359 | 2.4 |
| 87474 Kidney Cancer | 3.0 | Gastric Cancer Clontech 9060395 | 4.2 |
| (OD04622-01) | | | |
| 87475 Kidney NAT (OD04622-03) | 0.1 | NAT Stomach Clontech 9060394 | 1.6 |
| 85973 Kidney Cancer (OD04450- | 0.6 | Gastric Cancer Clontech 9060397 | 4.6 |
| 01) | | | |
| 85974 Kidney NAT (OD04450-03) | 0.3 | NAT Stomach Clontech 9060396 | 0.8 |
| Kidney Cancer Clontech 8120607 | 0.0 | Gastric Cancer GENPAK 064005 | 3.2 |

TABLE 35
Panel 3D

| | Relative | | Relative |
|---|---|---|---|
| | Expression (%) | | Expression (%) |
| | 3dx4tm6021t_a | | 3dx4tm6021t_a |
| Tissue Name | g2456_b2 | Tissue Name | g2456_b2 |
| 94905_Daoy_Medulloblastoma/ | 2.0 | 94954_Ca Ski_Cervical | 25.0 |
| Cerebellum_sscDNA | | epidermoid carcinoma | |
| | | (metastasis)_sscDNA | 25.0 |
| 94906_TE671_Medulloblastoma/ | 0.1 | 94955_ES-2_Ovarian clear cell | 24.1 |
| Cerebellum_sscDNA | | carcinoma_sscDNA | |
| 94907_D283 | 7.1 | 94957_Ramos/6h stim_Stimulated | 25.3 |
| Med_Medulloblastoma/ | | with PMA/ionomycin 6h_sscDNA | |
| Cerebellum_sscDNA | | | |
| 94908_PFSK-1_Primitive | 3.8 | 94958_Ramos/14h stim_{—} | 28.7 |
| Neuroectodermal/Cerebellum_{—} | | Stimulated with PMA/ionomycin | |
| sscDNA | | 14h_sscDNA | |
| 94909_XF-498_CNS_{—} | 0.0 | 94962_MEG-01_Chronic | |
| sscDNA | | myelogenous leukemia | |
| | | (megokaryoblast)_sscDNA | 48.4 |
| 94910_SNB- | 0.7 | 94963_Raji_Burkitt's | 13.1 |
| 78_CNS/glioma_sscDNA | | lymphoma_sscDNA | |
| 94911_SF- | 2.2 | 94964_Daudi_Burkitt's | 46.1 |
| 268_CNS/glioblastoma_sscDNA | | lymphoma_sscDNA | |
| 94912_T98G_Glioblastoma_sscD | 0.3 | 94965_U266_B-cell | 1.9 |
| NA | | plasmacytoma/myeloma_sscDNA | |
| 96776_SK-N-SH_Neuroblastoma | 7.4 | 94968_CA46_Burkitt's | 22.0 |
| (metastasis)_sscDNA | | lymphoma_sscDNA | |
| 94913_SF- | 0.7 | 94970_RL_non-Hodgkin's B-cell | 10.9 |
| 295_CNS/glioblastoma_sscDNA | | lymphoma_sscDNA | |
| 94914_Cerebellum_sscDNA | 7.1 | 94972_JM1_pre-B-cell | 23.6 |
| | | lymphoma/leukemia_sscDNA | |
| 96777_Cerebellum_sscDNA | 2.8 | 94973_Jurkat_T cell | 23.4 |
| | | leukemia_sscDNA | |
| 94916_NCI- | 28.3 | 94974_TF- | 19.1 |
| H292_Mucoepidermoid lung | | 1_Erythroleukemia_sscDNA | |
| carcinoma_sscDNA | | | |
| 94917_DMS-114_Small cell lung | 8.7 | 94975_HUT 78_T-cell | 21.3 |
| cancer_sscDNA | | lymphoma_sscDNA | |
| 94918_DMS-79_Small cell lung | 62.6 | 94977_U937_Histiocytic | 40.0 |
| cancer/neuroendocrine_sscDNA | | lymphoma_sscDNA | |
| 94919_NCI-H146_Small cell lung | 63.7 | 94980_KU-812_Myelogenous | 25.7 |
| cancer/neuroendocrine_sscDNA | | leukemia_sscDNA | |
| 94920_NCI-H526_Small cell lung | 90.5 | 94981_769-P_Clear cell renal | 5.5 |
| cancer/neuroendocrine_sscDNA | | carcinoma_sscDNA | |
| 94921_NCI-N417_Small cell lung | 0.3 | 94983_Caki-2_Clear cell renal | 26.3 |
| cancer/neuroendocrine_sscDNA | | carcinoma_sscDNA | |
| 94923_NCI-H82_Small cell lung | 1.7 | 94984_SW 839_Clear cell renal | 0.2 |
| cancer/neuroendocrine_sscDNA | | carcinoma_sscDNA | |
| 94924_NGI-H157_Squamous cell | 21.5 | 94986_G401_Wilms' | 4.2 |
| lung cancer (metastasis)_sscDNA | | tumor_sscDNA | |
| 94925_NCI-H1155_Large cell | 0.6 | 94987_Hs766T_Pancreatic | 2.7 |
| lung | | carcinoma (LN | |
| cancer/neuroendocrine_sscDNA | | metastasis)_sscDNA | |
| 94926_NCI-H1299_Large cell | 63.0 | 94988_CAPAN-1_Pancreatic | 4.3 |
| lung | | adenocarcinoma (liver | |
| cancer/neuroendocrine_sscDNA | | metastasis)_sscDNA | |
| 94927_NCI-H727_Lung | 100.0 | 94989_SU86.86_Pancreatic | 20.2 |
| carcinoid_sscDNA | | carcinoma (liver | |
| | | metastasis)_sscDNA | |
| 94928_NCI-UMC- 11_Lung | 1.4 | 94990_BxPC-3_Pancreatic | 6.8 |
| carcinoid_sscDNA | | adenocarcinoma_sscDNA | |
| 94929_LX-1_Small cell lung | 17.7 | 94991_HPAC_Pancreatic | 11.5 |
| cancer_sscDNA | | adenocarcinoma_sscDNA | |
| 94930_Colo-205_Colon | 14.5 | 94992_MIA PaCa-2 Pancreatic | 1.7 |
| cancer_sscDNA | | carcinoma_sscDNA | |
| 94931_KM12_Colon | 51.3 | 94993_CFPAC-1_Pancreatic | 17.5 |
| cancer_sscDNA | | ductal adenocarcinoma_sscDNA | |
| 94932_KM20L2_Colon | 5.2 | 94994_PANC-1_Pancreatic | 7.0 |
| cancer_sscDNA | | epithelioid ductal | |
| | | carcinoma_sscDNA | |
| 94933_NCI-H716_Colon | 63.6 | 94996_T24_Bladder carcinma | 16.7 |
| cancer_sscDNA | | (transitional cell)_sscDNA | |
| 94935_SW-48_Colon | 4.1 | 94997_5637_Bladder | 19.6 |
| adenocarcinoma_sscDNA | | carcinoma_sscDNA | |
| 94936_SW1116_Colon | 5.6 | 94998_HT-1197_Bladder | 13.2 |
| adenocarcinoma_sscDNA | | carcinoma_sscDNA | |
| 94937_LS 174T_Colon | 12.8 | 94999_UM-UC-3_Bladder | 5.7 |
| adenocarcinoma_sscDNA | | carcinma (transitional | |
| | | cell)_sscDNA | |
| 94938_SW-948_Colon | 2.1 | 95000_A204_Rhabdomyosarcoma_{—} | 1.6 |
| adenocarcinoma_sscDNA | | sscDNA | |
| 94939_SW-480_Colon | 7.0 | 95001_HT- | 3.3 |
| adenocarcinoma_sscDNA | | 1080_Fibrosarcoma_sscDNA | |
| 94940_NCI-SNU-5_Gastric | 6.8 | 95002_MG-63_Osteosarcoma | 3.0 |
| carcinoma_sscDNA | | (bone)_sscDNA | |
| 94941_KATO III_Gastric | 15.8 | 95003_SK-LMS- | 1.3 |
| carcinoma_sscDNA | | 1_Leiomyosarcoma | |
| | | (vulva)_sscDNA | |
| 94943_NCI-SNU-16_Gastric | 16.8 | 95004_SJRH30_Rhabdomyosarco | 0.2 |
| carcinoma_sscDNA | | ma (met to bone marrow)_sscDNA | |
| 94944_NCI-SNU-1_Gastric | 81.9 | 95005_A431_Epidermoid | 13.0 |
| carcinoma_sscDNA | | carcinoma_sscDNA | |
| 94946_RF-1_Gastric | 17.6 | 95007_WM266- | 0.7 |
| adenocarcinoma_sscDNA | | 4_Melanoma_sscDNA | |
| 94947_RF-48_Gastric | 27.2 | 95010_DU 145_Prostate | 0.3 |
| adenocarcinoma_sscDNA | | carcinoma (brain | |
| | | metastasis)_sscDNA | |
| 96778_MKN-45_Gastric | 6.7 | 95012_MDA-MB-468_Breast | 0.7 |
| carcinoma_sscDNA | | adenocarcinoma_sscDNA | |
| 94949_NCI-N87_Gastric | 18.3 | 95013_SCC-4_Squamous cell | 0.9 |
| carcinoma_sscDNA | | carcinoma of tongue_sscDNA | |
| 94951_OVCAR-5_Ovarian | 3.9 | 95014_SCC-9_Squamous cell | 0.6 |
| carcinoma_sscDNA | | carcinoma of tongue_sscDNA | |
| 94952_RL95-2_Uterine | 0.1 | 95015_SCC-15_Squamous cell | 0.5 |
| carcinoma_sscDNA | | carcinoma of tongue_sscDNA | |
| 94953_HelaS3_Cervical | 2.5 | 95017_CAL 27_Squamous cell | 9.9 |
| adenocarcinoma_sscDNA | | carcinoma of tongue_sscDNA | |

TABLE 36
Panel 4D

| | Relative | | Relative |
|---|---|---|---|
| | Expression (%) | | Expression (%) |
| | 4dtm3780t_{—} | | 4dtm3780t_{—} |
| Tissue Name | ag2456 | Tissue Name | ag2456 |
| 93768_Secondary Th1_anti- | 19.5 | 93100_HUVEC (Endothelial)_{—} | 6.0 |
| CD28/anti-CD3 | | IL-1b | |
| 93769_Secondary Th2_anti- | 11.1 | 93779_HUVEC (Endothelial)_{—} | 11.1 |
| CD28/anti-CD3 | | lFN gamma | |
| 93770_Secondary Tr1_anti- | 15.9 | 93102_HUVEC | 5.8 |
| CD28/anti-CD3 | | (Endothelial)_TNF alpha + IFN | |
| | | gamma | |
| 93573_Secondary Th1_resting day | 0.5 | 93101_HUVEC | 33.0 |
| 4-6 in IL-2 | | (Endothelial)_TNF alpha + IL4 | |
| 93572_Secondary Th2_resting day | 0.8 | 93781_HUVEC (Endothelial)_{—} | 4.2 |
| 4-6 in lL-2 | | IL-11 | |
| 93571_Secondary Tr1_resting day | 0.9 | 93583_Lung Microvascular | 15.2 |
| 4-6 in IL-2 | | Endothelial Cells_none | |
| 93568_primary Th1_anti- | 17.0 | 93584_Lung Microvascular | 20.0 |
| CD28/anti-CD3 | | Endothelial Cells_TNFa (4 ng/ml) | |
| | | and IL1b (1 ng/ml) | |
| 93569_primary Th2_anti- | 14.5 | 92662_Microvascular Dermal | 22.5 |
| CD28/anti-CD3 | | endothelium_none | |
| 93570_primary Tr1_anti- | 29.1 | 92663_Microsvasular Dermal | 9.5 |
| CD28/anti-CD3 | | endothelium_TNFa (4 ng/ml) and | |
| | | IL1b (1 ng/ml) | |
| 93565_primary Th1_resting dy 4-6 | 4.0 | 93773_Bronchial | 1.8 |
| in IL-2 | | epithelium_TNFa (4 ng/ml) and | |
| | | IL1b (1 ng/ml)** | 1.8 |
| 93566_primary Th2_resting dy 4-6 | 2.9 | 93347_Small Airway | 3.4 |
| in IL-2 | | Epithelium_none | |
| 93567_primary Tr1_resting dy 4-6 | 1.5 | 93348_Small Airway | 16.5 |
| in IL-2 | | Epithelium_TNFa (4 ng/ml) and | |
| | | IL1b (1 ng/ml) | |
| 93351_CD45RA CD4 | 8.0 | 92668_Coronery Artery | 3.0 |
| lymphocyte_anti-CD28/anti-CD3 | | SMC_resting | |
| 93352_CD45RO CD4 | 12.1 | 92669_Coronery Artery | 1.6 |
| lymphocyte_anti-CD28/anti-CD3 | | SMC_TNFa (4 ng/ml) and IL1b | |
| | | (1 ng/ml) | |
| 93251_CD8 Lymphocytes_anti- | 14.5 | 93107_astrocytes_resting | 3.7 |
| CD28/anti-CD3 | | | |
| 93353_chronic CD8 Lymphocytes | 8.8 | 93108_astrocytes_TNFa | 1.1 |
| 2ry_resting dy 4-6 in IL-2 | | (4 ng/ml) and IL1b (1 ng/ml) | |
| 93574_chronic CD8 Lymphocytes | 7.9 | 92666_KU-812 (Basophil)_{—} | 10.0 |
| 2ry_activated CD3/CD28 | | resting | |
| 93354_CD4_none | 0.4 | 92667_KU-812 | 18.9 |
| | | (Basophil)_PMA/ionoycin | |
| 93252_Secondary | 1.7 | 93579_CCD1106 | 4.8 |
| Th1/Th2/Tr1_anti-CD95 CH11 | | (Keratinocytes)_none | |
| 93103_LAK cells_resting | 9.5 | 93580_CCD1106 | 0.7 |
| | | (Keratinocytes)_TNFa and | |
| | | IFNg** | |
| 93788_LAK cells_IL-2 | 11.3 | 93791_Liver Cirrhosis | 0.4 |
| 93787_LAK cells_IL-2 + IL-12 | 6.7 | 93792_Lupus Kidney | 0.3 |
| 93789_LAK cells_IL-2 + IFN | 9.3 | 93577_NCI-H292 | 1.8 |
| gamma | | | |
| 93790_LAK cells_IL-2 + IL-18 | 11.3 | 93358_NCI-H292_IL-4 | 4.2 |
| 93104_LAK | 9.4 | 93360_NCI-H292_IL-9 | 6.7 |
| cell_PMA/ionomycin and IL-18 | | | |
| 93578_NK Cells IL-2_resting | 3.3 | 93359_NCI-H292_IL-13 | 2.0 |
| 93109_Mixed Lymphocyte | 4.8 | 93357_NCI-H292_IFN gamma | 3.0 |
| Reaction_Two Way MLR | | | |
| 93110_Mixed Lymphocyte | 6.5 | 93777_HPAEC_- | 9.7 |
| Reaction_Two Way MLR | | | |
| 93111_Mixed Lymphocyte | 4.4 | 93778_HPAEC_IL-1 beta/TNA | 9.0 |
| Reaction_Two Way MLR | | alpha | |
| 93112_Mononuclear Cells | 0.3 | 93254_Normal Human Lung | 1.1 |
| (PBMCs)_resting | | Fibroblast_none | |
| 93113_Mononuclear Cells | 30.1 | 93253_Normal Human Lung | 0.5 |
| (PBMCs)_PWM | | Fibroblast_TNFa (4 ng/ml) | |
| | | and IL-1b (1 ng/ml) | |
| 93114_Mononuclear Cells | 12.9 | 93257_Normal Human Lung | 5.6 |
| (PBMCs)_PHA-L | | Fibroblast_IL-4 | |
| 93249_Ramos (B cell)_none | 22.4 | 93256_Normal Human Lung | 3.7 |
| | | Fibroblast_IL-9 | |
| 93250_Ramos (B cell)_ionomycin | 73.2 | 93255_Normal Human Lung | 3.4 |
| | | Fibroblast_IL-13 | |
| 93349_B lymphocytes_PWM | 100.0 | 93258_Normal Human Lung | 3.9 |
| | | Fibroblast_IFN gamma | |
| 93350_B lymphoytes_CD40L and | 6.7 | 93106_Dermal Fibroblasts | 6.2 |
| IL-4 | | CCD1070_resting | |
| 92665_EOL-1 | 5.5 | 93361_Dermal Fibroblasts | 10.7 |
| (Eosinophil)_dbcAMP | | CCD1070_TNF alpha 4 ng/ml | |
| differentiated | | | |
| 93248_EOL-1 | 3.6 | 93105_Dermal Fibroblasts | 2.4 |
| (Eosinophil)_dbcAMP/ | | CCD1070_IL-1 beta 1 ng/ml | |
| PMAionomycin | | | |
| 93356_Dendritic Cells_none | 17.3 | 93772_dermal fibroblast_IFN | 1.6 |
| | | gamma | |
| 93355_Dendritic Cells_LPS | 16.8 | 93771_dermal fibroblast_IL-4 | 3.0 |
| 100 ng/ml | | | |
| 93775_Dendritic Cells_anti-CD40 | 21.9 | 93260_IBD Colitis 2 | 0.4 |
| 93774_Monocytes_resting | 0.5 | 93261_IBD Crohns | 0.4 |
| 93776_Monocytes_LPS 50 ng/ml | 1.1 | 735010_Colon_normal | 2.9 |
| 93581_Macrophages_resting | 39.2 | 735019_Lung_none | 6.7 |
| 93582_Macrophages_LPS 100 | 3.7 | 64028-1_Thymus_none | 1.7 |
| ng/ml | | | |
| 93098_HUVEC | 11.7 | 64030-1_Kidney_none | 6.4 |
| (Endothelial)_none | | | |
| 93099_HUVEC | 18.8 | | |
| (Endothelial)_starved | | | |

TABLE 37
Panel CNS_neurodegeneration_v1.0

| | Relative | | Relative |
|---|---|---|---|
| | Expression (%) | | Expression (%) |
| | tm7017t_{—} | | tm7017t_ag |
| Tissue Name | ag2456_b2_s1 | Tissue Name | 2456_b2_s1 |
| AD 1 Hippo | 0.0 | Control (Path) 3 Temporal | 0.0 |
| | | Ctx | |
| AD 2 Hippo | 0.0 | Control (Path) 4 Temporal | 0.1 |
| | | Ctx | |
| AD 3 Hippo | 0.0 | AD 1 Occipital Ctx | 0.0 |
| AD 4 Hippo | 0.0 | AD 2 Occipital Ctx (Missing) | 0.0 |
| AD 5 Hippo | 100.0 | AD 3 Occipital Ctx | 0.0 |
| AD 6 Hippo | 0.5 | AD 4 Occipital Ctx | 0.0 |
| Control 2 Hippo | 0.0 | AD 5 Occipital Ctx | 99.2 |
| Control 4 Hippo | 0.0 | AD 6 Occipital Ctx | 2.4 |
| Control (Path) 3 Hippo | 0.2 | Control 1 Occipital Ctx | 0.0 |
| AD 1 Temporal Ctx | 0.0 | Control 2 Occipital Ctx | 0.0 |
| AD 2 Temporal Ctx | 0.0 | Control 3 Occipital Ctx | 0.0 |
| AD 3 Temporal Ctx | 0.0 | Control 4 Occipital Ctx | 0.0 |
| AD 4 Temporal Ctx | 0 | Control (Path) 1 Occipital Ctx | 0.1 |
| AD 5 Inf Temporal Ctx | 46.2 | Control (Path) 2 Occipital Ctx | 0.0 |
| AD 5 Sup Temporal Ctx | 99.2 | Control (Path) 3 Occipital Ctx | 0.0 |
| AD 6 Inf Temporal Ctx | 0.5 | Control (Path) 4 Occipital Ctx | 0.0 |
| AD 6 Sup Temporal Ctx | 0.4 | Control 1 Parietal | 0.0 |
| Control 1 Temporal Ctx | 0.0 | Control 2 Parietal | 0.1 |
| Control 2 Temporal Ctx | 0.0 | Control 3 Parietal | 0.0 |
| Control 3 Temporal Ctx | 0.0 | Control (Path) 1 Parietal | 0.1 |
| Control 3 Temporal Ctx | 0.0 | Control (Path) 2 Parietal | 0.0 |
| Control (Path) 1 Temporal Ctx | 0.0 | Control (Path) 3 Parietal | 0.0 |
| Control (Path) 2 Temporal Ctx | 0.0 | Control (Path) 4 Parietal | 0.1 |

Panel 1.3D Summary: Ag2456 Expression of the NOV5 gene is highest in melanoma (CT=26.4) and is expressed at moderate to high levels across all melanoma cancer cell lines present in this panel. This expression profile strongly suggests that the NOV5 gene could be used to distinguish melanoma cell lines from other tissue samples.

Panel 1.3D also shows that the NOV5 gene is expressed at high to moderate levels in the brain. Among CNS samples, this gene is expressed at highest levels (CT=26.9) in the hippocampus region of the brain. Expression is also detected in the cerebral cortex, cerebellum, substantia nigra, thalamus, amygdala, and spinal cord. The NOV5 gene encodes a protein with homology to fatty acid binding proteins. Fatty acids are ubiquitious in central nervous system associated membranes such as myelin, synaptic vesicles, pre- and post-synaptic membranes, and synaptosomal cytosol, where they play a critical role in membrane composition and fluidity. Therefore, the fatty acid binding proteins that transport the hydrophobic fatty acids into the cell play an important role both during development and during dendritic outgrowth repair, axonal extension, and compensatory synaptogenesis. Fatty acid transport proteins are upregulated during the response to injury, and the decrease in levels in aged mammals may be partially responsible for their decreased ability to respond to and repair CNS injury. Thus, the Gmba38818_A protein product may play a role in some or all of these central nervous system related processes and therapeutic modulation of the gene product could be important in treating these same disease processes.

This gene is also widely expressed at moderate levels in most metabolic tissues, including adipose, adrenal gland, adult and fetal heart, adult and fetal liver, adult and skeletal muscle, pancreas (CT=31), pituitary and thyroid. Therefore, therapeutic targeting of the fatty acid binding protein encoded by the NOV5 gene may be useful for the treatment of metabolic diseases, such as obesity and diabetes.

Panel 2D Summary Ag2456 Expression of the NOV5 gene is highest in lung cancer (CT=23.1). Overexpression of the NOV5 gene is seen in 3/5 lung cancer samples when compared to their normal adjacent tissue counterparts. Thus, based on this expression profile, the expression of the NOV5 gene could be used to distinguish lung cancer samples from normal lung. In addition, therapeutic modulation of the NOV5 gene product, through the use of small molecule drugs or antibodies, could be beneficial in the treatment of lung cancer.

Panel 3D Summary Ag2456 Expression of the NOV5 gene is highest in samples derived from colon cancer (CT=29), and lung cancer (CT=28.3) cell lines. Overexpression of this gene in lung cancers is consistent with the results in panel 2D. Thus, based on this expression pattern, the NOV5 gene could be used to distinguish lung cancer cell lines from other cell lines. In addition, therapeutic modulation of this gene product, through the use of small molecule drugs or antibodies, could potentially be effective in the treatment of lung cancer.

Panel 4D Summary Ag2456 Expression of the NOV5 gene is highest in primary B cells activated by PWM (CT=24.4), and in an activated B cell line, Ramos (CT=24.9). The expression of the Gmba38818_A gene in PBMC treated with the B cell mitogen, PWM, (CT=26.2) is consistent with this data. This gene probably encodes for a fatty acid binding protein that might be involved in B cell trafficking. Thus, drug targeting of the fatty acid binding protein encoded by the NOV5 gene may be valuable for treatment of immune disease processes, particularly autoimmune diseases such as lupus, rheumatoid arthritis, and diseases associated with hyperglobulinemia.

Panel CNS_neurodegeneration_v1.0 Summary Ag2456 Expression of the NOV5 gene is highest in tissue samples derived from different brain regions of a patient with Alzheimer's disease. These regions include the hippocampus (CT=19.4), the superior temporal cortex (CT=19.5), the inferior temporal cortex (CT=20.6), and the occipital cortex (CT=19.5). Thus, this gene may be involved in the pathology of at least one form of Alzheimer's disease. Upregulation of the NOV5 gene or its protein product may be of use in enhancing compensatory synatogenesis and axon or dendritic outgrowth in response to spinal cord injury, neuronal death resulting from stroke or head trauma, or neurodegeneration present in Alzheimer's, Parkinson's, Huntington's, spinocerebellar ataxia, progressive supranuclear palsy. (Glatz et al., J Mol Neurosci 16:23-32, 2001; Pu et al., Mol Cell Biochem 198:69-78, 1999; Liu et al., J Neurosci Res 48:551-62, 1997.)

NOV6a: Neurolysin Precursor-like

Expression of the NOV6a gene (SC133790496_A) was assessed using the primer-probe set Ag2458, described in Table 38. Results of the RTQ-PCR runs are shown in Tables 39, 40, 41, 42, 43, 44, and 45.

TABLE 38

Probe Name Ag2458

| | | | | | |
|---|---|---|---|---|---|
| | | | | Start | |
| Primers | Sequences | TM | Length | Position | SEQ ID NO: |

| | | | | | |
|---|---|---|---|---|---|
| Forward | 5′-GTTGGTGGTTCCAGGATTTT-3′ | 58.7 | 20 | 58 | 167 |
| Probe | TET-5′- | 66.6 | 26 | 104 | 168 |
| | TGATGTCTCCTCTTCAGGCAATGTCT-3′ | | | | |
| | TAMRA | | | | |
| Reverse | 5′-CTGCCAGCCACAGTATAGGA-3′ | 58.9 | 20 | 130 | 169 |

TABLE 39
Panel 1.3D

| | Relative Expression(%) | |
|---|---|---|
| | 1.3dtm4270t_{—} | 1.3dx4tm5407t_{—} |
| Tissue Name | ag2458 | ag2458_a2 |
| Liver adenocarcinoma | 33.9 | 41.4 |
| Pancreas | 1.3 | 0.8 |
| Pancreatic ca. CAPAN 2 | 6.9 | 44.3 |
| Adrenal gland | 0.7 | 1.1 |
| Thyroid | 2.8 | 3.3 |
| Salivary gland | 0.6 | 2 |
| Pituitary gland | 1.6 | 0.4 |
| Brain (fetal) | 9.2 | 19.7 |
| Brain (whole) | 5.4 | 17.7 |
| Brain (amygdala) | 6.7 | 9.0 |
| Brain (cerebellum) | 2.7 | 10.2 |
| Brain (hippocampus) | 23 | 13.1 |
| Brain (substantia nigra) | 1.6 | 7.2 |
| Brain (thalamus) | 4.9 | 16.6 |
| Cerebral Cortex | 26.2 | 8.2 |
| Spinal cord | 2.4 | 9.8 |
| CNS ca. (glio/astro) U87-MG | 11.0 | 19.7 |
| CNS ca. (glio/astro) U-118-MG | 45.1 | 82.4 |
| CNS ca. (astro) SW1783 | 21.9 | 46.0 |
| CNS ca.* (neuro; met) SK-N-AS | 73.7 | 40.2 |
| CNS ca. (astro) SF-539 | 9.9 | 12.0 |
| CNS ca. (astro) SNB-75 | 22.5 | 71.5 |
| CNS ca. (glio) SNB-19 | 6.2 | 18.1 |
| CNS ca. (glio) U251 | 5.9 | 45.0 |
| CNS ca. (glio) SF-295 | 13.0 | 26.4 |
| Heart (fetal) | 1.7 | 0.0 |
| Heart | 1.2 | 4.4 |
| Fetal Skeletal | 11.4 | 1.2 |
| Skeletal muscle | 3.8 | 41.7 |
| Bone marrow | 1.5 | 2.2 |
| Thymus | 0.8 | 0.4 |
| Spleen | 0.9 | 0.8 |
| Lymph node | 1.3 | 10.4 |
| Colorectal | 4.8 | 3.1 |
| Stomach | 0.0 | 3.1 |
| Small intestine | 1.1 | 2.3 |
| Colon ca. SW480 | 19.5 | 18.9 |
| Colon ca.* (SW480 met)SW620 | 29.9 | 29.8 |
| Colon ca. HT29 | 17.7 | 9.7 |
| Colon ca. HCT-116 | 22.1 | 43.8 |
| Colon ca. CaCo-2 | 13.5 | 18.4 |
| 83219 CC Well to Mod Diff | 10.6 | 7.0 |
| (ODO3866) | | |
| Colon ca. HCC-2998 | 45.7 | 21.1 |
| Gastric ca.* (liver met) NCI-N87 | 38.4 | 69.5 |
| Bladder | 7.4 | 13.6 |
| Trachea | 2.9 | 3.0 |
| Kidney | 1.3 | 3.5 |
| Kidney (fetal) | 3.6 | 3.9 |
| Renal ca. 786-0 | 10.0 | 19.0 |
| Renal ca. A498 | 29.7 | 28.6 |
| Renal ca. RXF 393 | 5.6 | 53.0 |
| Renal ca. ACHN | 5.5 | 13.3 |
| Renal ca. UO-31 | 21.6 | 45.5 |
| Renal ca. TK-10 | 20.4 | 27.0 |
| Liver | 2.9 | 2.4 |
| Liver (fetal) | 3.8 | 5.0 |
| Liver ca. (hepatoblast) HepG2 | 17.3 | 28.2 |
| Lung | 1.9 | 2.5 |
| Lung (fetal) | 1.4 | 5.6 |
| Lung ca. (small cell) LX-1 | 11.8 | 40.6 |
| Lung ca. (small cell) NCI-H69 | 31.4 | 44.0 |
| Lung ca. (s.cell var.) SHP-77 | 69.3 | 90.5 |
| Lung ca. (large cell)NCI-H460 | 9.9 | 63.6 |
| Lung ca. (non-sm. cell) A549 | 20.9 | 25.9 |
| Lung ca. (non-s.cell) NCI-H23 | 11.3 | 10.7 |
| Lung ca (non-s.cell) HOP-62 | 4.1 | 9.1 |
| Lung ca. (non-s.cl) NCI-H522 | 29.1 | 30.0 |
| Lung ca. (squam.) SW 900 | 9.7 | 20.1 |
| Lung ca. (squam.) NCI-H596 | 9.5 | 43.2 |
| Mammary gland | 5.3 | 9.0 |
| Breast ca.* (pl. effusion) MCF-7 | 14.3 | 30.8 |
| Breast ca.* (pl.ef) MDA-MB-231 | 42.3 | 41.1 |
| Breast ca.* (pl. effusion) T47D | 9.6 | 13.8 |
| Breast ca. BT-549 | 100.0 | 100.0 |
| Breast ca. MDA-N | 17.2 | 8.8 |
| Ovary | 4.8 | 0.9 |
| Ovarian ca. OVCAR-3 | 11.3 | 20.9 |
| Ovarian ca. OVCAR-4 | 2.5 | 10.6 |
| Ovarian ca. OVCAR-5 | 20.0 | 26.3 |
| Ovarian ca. OVCAR-8 | 18.6 | 16.5 |
| Ovarian ca. IGROV-1 | 6.9 | 6.8 |
| Ovarian ca.* (ascites) SK-OV-3 | 32.3 | 64.3 |
| Uterus | 1.8 | 4.3 |
| Placenta | 2.2 | 1.1 |
| Prostate | 2.0 | 2.4 |
| Prostate ca.* (bone met)PC-3 | 3.1 | 3.2 |
| Testis | 2.0 | 1.5 |
| Melanoma Hs688(A).T | 4.6 | 5.4 |
| Melanoma* (met) Hs688(B).T | 2.4 | 6.9 |
| Melanoma UACC-62 | 1.3 | 12.6 |
| Melanoma M14 | 5.9 | 56.2 |
| Melanoma LOX IMVI | 14.7 | 8.1 |
| Melanoma* (met) SK-MEL-5 | 16.2 | 24.7 |
| Adipose | 4.4 | 3.4 |

TABLE 40
Panel 2D

| | Relative |
|---|---|
| | Expression(%) |
| | 2dtm4271t_{—} |
| Tissue Name | ag2458 |
| Normal Colon GENPAK 061003 | 62.4 |
| 83219 CC Well to Mod Diff | 16.8 |
| (ODO3866) | |
| 83220 CC NAT (ODO3866) | 11.7 |
| 83221 CC Gr.2 rectosigmoid | 18.6 |
| (ODO3868) | |
| 83222 CC NAT (ODO3868) | 8.4 |
| 83235 CC Mod Diff (ODO3920) | 34.2 |
| 83236 CC NAT (ODO3920) | 11.3 |
| 83237 CC Gr.2 ascend colon | 74.7 |
| (ODO3921) | |
| 83238 CC NAT (ODO3921) | 9.6 |
| 83241 CC from Partial | 35.6 |
| Hepatectomy (ODO4309) | |
| 83242 Liver NAT (ODO4309) | 32.3 |
| 87472 Colon mets to lung | 29.7 |
| (OD04451-01) | |
| 87473 Lung NAT (OD04451-02) | 6.4 |
| Normal Prostate Clontech A+ | 8.8 |
| 6546-1 | |
| 84140 Prostate Cancer | 25.9 |
| (OD04410) | |
| 84141 Prostate NAT (OD04410) | 27.9 |
| 87073 Prostate Cancer | 15.8 |
| (OD04720-01) | |
| 87074 Prostate NAT (OD04720- | 28.1 |
| 02) | |
| Normal Lung GENPAK 061010 | 24.8 |
| 83239 Lung Met to Muscle | 100.0 |
| (ODO4286) | |
| 83240 Muscle NAT (ODO4286) | 31.9 |
| 84136 Lung Malignant Cancer | 29.5 |
| (OD03126) | |
| 84137 Lung NAT (OD03126) | 27.9 |
| 84871 Lung Cancer (OD04404) | 71.7 |
| 84872 Lung NAT (OD04404) | 15.1 |
| 84875 Lung Cancer (OD04565) | 28.5 |
| 84876 Lung NAT (OD04565) | 13.1 |
| 85950 Lung Cancer (OD04237- | 90.1 |
| 01) | |
| 85970 Lung NAT (OD04237-02) | 16.8 |
| 83255 Ocular Mel Met to Liver | 76.3 |
| (ODO4310) | |
| 83256 Liver NAT (ODO4310) | 26.8 |
| 84139 Melanoma Mets to Lung | 36.3 |
| (OD04321) | |
| 84138 Lung NAT (OD04321) | 22.5 |
| Normal Kidney GENPAK | 33.7 |
| 061008 | |
| 83786 Kidney Ca, Nuclear grade | 68.8 |
| 2 (OD04338) | |
| 83787 Kidney NAT (OD04338) | 33.4 |
| 83788 Kidney Ca Nuclear grade | 30.6 |
| 1/2 (OD04339) | |
| 83789 Kidney NAT (OD04339) | 27.2 |
| 83790 Kidney Ca, Clear cell type | 49.3 |
| (OD04340) | |
| 83791 Kidney NAT (OD04340) | 32.1 |
| 83792 Kidney Ca, Nuclear grade | 16.0 |
| 3 (OD04348) | |
| 83793 Kidney NAT (OD04348) | 35.4 |
| 87474 Kidney Cancer | 13.2 |
| (OD04622-01) | |
| 87475 Kidney NAT (OD04622- | 4.0 |
| 03) | |
| 85973 Kidney Cancer | 48.6 |
| (OD04450-01) | |
| 85974 Kidney NAT (OD04450- | 30.4 |
| 03) | |
| Kidney Cancer Clontech | 20.4 |
| 8120607 | |
| Kidney NAT Clontech 8120608 | 7.3 |
| Kidney Cancer Clontech 8120613 | 14.4 |
| Kidney NAT Clontech 8120614 | 8.8 |
| Kidney Cancer Clontech 9010320 | 12.2 |
| Kidney NAT Clontech 9010321 | 22.1 |
| Normal Uterus GENPAK 061018 | 8.3 |
| Uterus Cancer GENPAK 064011 | 15.3 |
| Normal Thyroid Clontech A+ | 15.1 |
| 6570-1 | |
| Thyroid Cancer GENPAK 064010 | 33.0 |
| Thyroid Cancer INVITROGEN | 21.6 |
| A302152 | |
| Thyroid NAT INVITROGEN | 14.4 |
| A302153 | |
| Normal Breast GENPAK 061019 | 33.2 |
| 84877 Breast Cancer (OD04566) | 44.8 |
| 85975 Breast Cancer (OD04590- | 95.9 |
| 01) | |
| 85976 Breast Cancer Mets | 61.1 |
| (OD04590-03) | |
| 87070 Breast Cancer Metastasis | 38.4 |
| (OD04655-05) | |
| GENPAK Breast Cancer 064006 | 33.2 |
| Breast Cancer Res. Gen. 1024 | 23.0 |
| Breast Cancer Clontech 9100266 | 33.4 |
| Breast NAT Clontech 9100265 | 19.5 |
| Breast Cancer INVITROGEN | 47.0 |
| A209073 | |
| Breast NAT INVITROGEN | 37.6 |
| A2090734 | |
| Normal Liver GENPAK 061009 | 15.5 |
| Liver Cancer GENPAK 064003 | 14.0 |
| Liver Cancer Research Genetics | 20.4 |
| RNA 1025 | |
| Liver Cancer Research Genetics | 7.1 |
| RNA 1026 | |
| Paired Liver Cancer Tissue | 24.8 |
| Research Genetics RNA 6004-T | |
| Paired Liver Tissue Research | 18.0 |
| Genetics RNA 6004-N | |
| Paired Liver Cancer Tissue | 13.4 |
| Research Genetics RNA 6005-T | |
| Paired Liver Tissue Research | 7.3 |
| Genetics RNA 6005-N | |
| Normal Bladder GENPAK 061001 | 59.9 |
| Bladder Cancer Research Genetics | 5.2 |
| RNA 1023 | |
| Bladder Cancer INVITROGEN | 30.1 |
| A302173 | |
| 87071 Bladder Cancer (OD04718- | 65.5 |
| 01) | |
| 87072 Bladder Normal Adjacent | 18.4 |
| (OD04718-03) | |
| Normal Ovary Res. Gen. | 5.1 |
| Ovarian Cancer GENPAK 064008 | 29.1 |
| 87492 Ovary Cancer (OD04768- | 66.0 |
| 07) | |
| 87493 Ovary NAT (OD04768-08) | 7.7 |
| Normal Stomach GENPAK | 16.4 |
| 061017 | |
| Gastric Cancer Clontech 9060358 | 3.5 |
| NAT Stomach Clontech 9060359 | 10.5 |
| Gastric Cancer Clontech 9060395 | 29.9 |
| NAT Stomach Clontech 9060394 | 12.9 |
| Gastric Cancer Clontech 9060397 | 42.3 |
| NAT Stomach Clontech 9060396 | 6.2 |
| Gastric Cancer GENPAK 064005 | 31.0 |

TABLE 41
Panel 3D

| | Relative | | Relative |
|---|---|---|---|
| | Expression (%) | | Expression (%) |
| | 3dx4tm5121t_{—} | | 3dx4tm5121t_{—} |
| Tissue Name | ag2458_b2 | Tissue Name | ag2458_b2 |
| 94905_Daoy_Medulloblastoma/ | 13.6 | 94954_Ca Ski_Cervical | 24.3 |
| Cerebellum_sscDNA | | epidermoid carcinoma | |
| | | (metastasis)_sscDNA | |
| 94906_TE671_Medulloblastom/ | 7.4 | 94955_ES-2_Ovarian clear cell | 16.1 |
| Cerebellum_sscDNA | | carcinoma_sscDNA | |
| 94907_D283 | 53.0 | 94957_Ramos/6h stim_Stimulated | 8.7 |
| Med_Medulloblastoma/ | | with PMA/ionomycin 6h_sscDNA | |
| Cerebellum_sscDNA | | | |
| 94908_PFSK-1_Primitive | 6.5 | 94958_Ramos/14h stim_{—} | 8.3 |
| Neuroectodermal/Cerebellum_{—} | | Stimulated with PMA/ionomycin | |
| sscDNA | | 14h_sscDNA | |
| 94909_XF-498_CNS_sscDNA | 6.8 | 94962_MEG-01_Chronic | 22.1 |
| | | myelogenous leukemia | |
| | | (megokaryoblast)_sscDNA | |
| 94910_SNB-78_{—} | 9.8 | 94963_Raji_Burkitt's | 8.3 |
| CNS/glioma_sscDNA | | lymphoma_sscDNA | |
| 94911_SF-268_{—} | 10.2 | 94964_Daudi_Burkitt's | 19.8 |
| CNS/glioblastoma_sscDNA | | lymphoma_sscDNA | |
| 94912_T98G_Glioblastoma_{—} | 15.7 | 94965_U266_B-cell | 6.4 |
| sscDNA | | plasmacytoma/myeloma_sscDNA | |
| 96776_SK-NSH_Neuroblastoma | 16.5 | 94968_CA46_Burkitt's | 6.5 |
| (metastasis)_sscDNA | | lymphoma_sscDNA | |
| 94913_SF-295_{—} | 7.4 | 94970_RL_non-Hodgkin's B-cell | 9.0 |
| CNS/glioblastoma_sscDNA | | lymphoma_sscDNA | |
| 94914_Cerebellum_sscDNA | 3.9 | 94972_JM1_pre-B-cell | 4.1 |
| | | lymphoma/leukemia_sscDNA | |
| 96777_Cerebellum_sscDNA | 0.8 | 94973 _Jurkat_T cell | 9.8 |
| | | leukemia_sscDNA | |
| 94916_NCI- | 46.9 | 94974_TF1_Erythroleukemia_{—} | 31.5 |
| H292_Mucoepidermoid lung | | sscDNA | |
| carcinoma_sscDNA | | | |
| 94917_DM5-114_Small cell | 10.9 | 94975_HUT 78_T-cell | 15.7 |
| lung cancer_sscDNA | | lymphoma_sscDNA | |
| 94918_DMS-79_Small cell lung | 100.0 | 94977_U937_Histiocytic | 30.5 |
| cancer/neuroendocrine_sscDNA | | lymphoma_sscDNA | |
| 94919_NCI-H146_Small cell | 33.4 | 94980_KU-812_Myelogenous | 21.7 |
| lung | | leukemia_sscDNA | |
| cancer/neuroendocrine_sscDNA | | | |
| 94920_NCI-H526_Small cell | 30.2 | 94981_769-P_Clear cell renal | 16.6 |
| lung | | carcinoma_sscDNA | |
| cancer/neuroendocrine_sscDNA | | | |
| 94921_NCI-N417_Small cell | 26.1 | 94983_Caki-2_Clear cell renal | 16.8 |
| lung | | carcinoma_sscDNA | |
| cancer/neuroendocrine_sscDNA | | | |
| 94923_NCI-H82_Small cell lung | 28.4 | 94984_SW 839_Clear cell renal | 11.8 |
| cancer/neuroendocrine_sscDNA | | carcinoma_sscDNA | |
| 94924_NCI-H157_Squamous | 88.0 | 94986_G401_Wilms' | 16.5 |
| cell lung cancer | | tumor_sscDNA | |
| (metastasis)_sscDNA | | | |
| 94925_NCI-H1155_Large cell | 31.3 | 94987_Hs766T_Pancreatic | 12.9 |
| lung | | carcinoma (LN | |
| cancer/neuroendocrine_sscDNA | | metastasis)_sscDNA | |
| 94926_NCI-H1299_Large cell | 32.4 | 94988_CAPAN-1_Pancreatic | 12.0 |
| lung | | adenocarcinoma (liver | |
| cancer/neuroendocrine_sscDNA | | metastasis)_sscDNA | |
| 94927_NCI-H727_Lung | 23.0 | 94989_SU86.86_Pancreatic | 32.6 |
| carcinoid_sscDNA | | carcinoma (liver | |
| | | metastasis)_sscDNA | |
| 94928_NCI-UMC-11_Lung | 75.0 | 94990_BxPC-3_Pancreatic | 4.0 |
| carcinoid_sscDNA | | adenocarcinoma_sscDNA | |
| 94929_LX-1_Small cell lung | 17.0 | 94991_HPAC_Pancreatic | 26.4 |
| cancer_sscDNA | | adenocarcinoma_sscDNA | |
| 94930_Colo-205_Colon | 11.1 | 94992_MIA PaCa-2_Pancreatic | 8.2 |
| cancer_sscDNA | | carcinoma_sscDNA | |
| 94931_KM12_Colon | 37.3 | 94993_CFPAC-1_Pancreatic | 30.3 |
| cancer_sscDNA | | ductal adenocarcinoma_sscDNA | |
| 94932_KM20L2_Colon | 7.8 | 94994_PANC-1_Pancreatic | 26.7 |
| cancer_sscDNA | | epithelioid ductal | |
| | | carcinoma_sscDNA | |
| 94933_NCI-H716_Colon | 32.3 | 94996_T24_Bladder carcinma | 8.2 |
| cancer_sscDNA | | (transitional cell)_sscDNA | |
| 94935_SW-48_Colon | 7.8 | 94997_5637_Bladder | 20.5 |
| adenocarcinoma_sscDNA | | carcinoma_sscDNA | |
| 94936_SW1116_Colon | 10.9 | 94998_HT-1197_Bladder | 18.0 |
| adenocarcinoma_sscDNA | | carcinoma_sscDNA | |
| 94937_LS 174T_Colon | 29.3 | 94999_UM-UC-3_Bladder | 4.6 |
| adenocarcinoma _sscDNA | | carcinma (transitional | |
| | | cell)_sscDNA | |
| 94938_SW-948_Colon | 1.9 | 95000_A204_Rhabdomyosarcoma | 20.0 |
| adenocarcinoma_sscDNA | | _sscDNA | |
| 94939_SW-480_Colon | 4.9 | 95001_HT-1080_{—} | 15.4 |
| adenocarcinoma_sscDNA | | Fibrosarcoma_sscDNA | |
| 94940_NCI-SNU-5_Gastric | 8.3 | 95002_MG-63_Osteosarcoma | 16.2 |
| carcinoma_sscDNA | | (bone)_sscDNA | |
| 94941_KATO III_Gastric | 53.1 | 95003_SK-LMS-1_Leiomyosarcoma | 27.5 |
| carcinoma_sscDNA | | (vulva)_sscDNA | |
| 94943_NCI-SNU-16_Gastric | 7.3 | 95004_SJRH30_Rhabdomyosarcoma | 12.5 |
| carcinoma_sscDNA | | (met to bone marrow)_sscDNA | |
| 94944_NCI-SNU-1_Gastric | 64.4 | 95005_A431_Epidermoid | 6.9 |
| carcinoma_sscDNA | | carcinoma_sscDNA | |
| 94946_RF-1_Gastric | 11.4 | 95007_WM2664_{—} | 7.5 |
| adenocarcinoma_sscDNA | | Melanoma_sscDNA | |
| 94947_RF-48_Gastric | 15.4 | 95010_DU 145_Prostate | 0.1 |
| adenocarcinoma_sscDNA | | carcinoma (brain | |
| | | metastasis)_sscDNA | |
| 96778_MKN-45_Gastric | 28.8 | 95012_MDA-MB-468_Breast | 13.5 |
| carcinoma_sscDNA | | adenocarcinoma_sscDNA | |
| 94949_NCI-N87_Gastric | 19.5 | 95013_SCC-4_Squamous cell | 1.3 |
| carcinoma_sscDNA | | carcinoma of tongue_sscDNA | |
| 94951_OVCAR-5_Ovarian | 11.7 | 95014_SCC-9_Squamous cell | 0.3 |
| carcinoma_sscDNA | | carcinoma of tongue_sscDNA | |
| 94952_RL95-2_Uterine | 4.5 | 95015_SCC-15_Squamous cell | 0.3 |
| carcinoma_sscDNA | | carcinoma of tongue_sscDNA | |
| 94953_HelaS3_Cervical | 11.3 | 95017_CAL 27_Squamous cell | 24.5 |
| adenocarcinoma_sscDNA | | carcinoma of tongue_sscDNA | |

TABLE 42
Panel 4D

| | Relative | | Relative |
|---|---|---|---|
| | Expression(%) | | Expression(%) |
| Tissue Name | 4dtm4272t_ag2458 | Tissue Name | 4dtm4272t_ag2458 |
| 93768_Secondary Th1_anti- | 14.5 | 93100_HUVEC (Endothelial)_IL- | 16.5 |
| CD28/anti-CD3 | | 1b | |
| 93769_Secondary Th2_anti- | 7.4 | 93779_HUVEC (Endothelial)_IFN | 26.1 |
| CD28/anti-CD3 | | gamma | |
| 93770_Secondary Tr1_anti- | 9.5 | 93102_HUVEC | 16.0 |
| CD28/anti-CD3 | | (Endothelial)_TNF alpha + IFN | |
| | | gamma | |
| 93573_Secondary Th1_resting | 0.2 | 93101_HUVEC | 23.2 |
| day 4-6 in IL-2 | | (Endothelial)_TNF alpha + IL4 | |
| 93572_Secondary Th2_resting | 0.6 | 93781_HUVEC (Endothelial)_IL- | 13.6 |
| day 4-6 in IL-2 | | 11 | |
| 93571_Secondary Tr1_resting | 0.8 | 93583_Lung Microvascular | 21.0 |
| day 4-6 in IL-2 | | Endothelial Cells_none | |
| 93568_primary Th1_anti- | 19.8 | 93584_Lung Microvascular | 18.3 |
| CD28/anti-CD3 | | Endothelial Cells_TNFa (4 ng/ml) | |
| | | and IL1b (1 ng/ml) | |
| 93569_primary Th2_anti- | 13.0 | 92662_Microvascular Dermal | 35.4 |
| CD28/anti-CD3 | | endothelium_none | |
| 93570_primary Tr1 anti- | 19.2 | 92663 Microsvasular Dermal | 20.0 |
| CD28/anti-CD3 | | endothelium_TNFa (4 ng/ml) and | |
| | | IL1b (1 ng/ml) | |
| 93565_primary Th1_resting dy | 8.8 | 93773_Bronchial | 14.9 |
| 4-6 in IL-2 | | epithelium_TNFa (4 ng/ml) and | |
| | | IL1b (1 ng/ml) ** | |
| 93566_primary Th2_resting dy | 2.2 | 93347_Small Airway | 7.2 |
| 4-6 in IL-2 | | Epithelium_none | |
| 93567_primary Tr1_resting dy 4- | 3.6 | 93348_Small Airway | 45.4 |
| 6 in IL-2 | | Epithelium_TNFa (4 ng/ml) and | |
| | | IL1b (1 ng/ml) | |
| 93351_CD45RA CD4 | 19.5 | 92668_Coronery Artery | 17.7 |
| lymphocyte_anti-CD28/anti- | | SMC_resting | |
| CD3 | | | |
| 93352_CD45RO CD4 | 17.7 | 92669_Coronery Artery | 10.8 |
| lymphocyte_anti-CD28/anti- | | SMC_TNFa (4 ng/ml) and IL1b (1 | |
| CD3 | | ng/ml) | |
| 93251_CD8 Lymphocytes_anti- | 9.3 | 93107_astrocytes_resting | 11.1 |
| CD28/anti-CD3 | | | |
| 93353_chronic CD8 | 11.0 | 93108_astrocytes_TNFa (4 ng/ml) | 10.5 |
| Lymphocytes 2ry_resting dy 4-6 | | and IL1b (1 ng/ml) | |
| in IL-2 | | | |
| 93574_chronic CD8 | 5.5 | 92666_KU-812 (Basophil)_resting | 18.8 |
| Lymphocytes 2ry_activated | | | |
| CD3/CD28 | | | |
| 93354_CD4_none | 0.7 | 92667_KU-812 | 27.9 |
| | | (Basophil)_PMA/ionoycin | |
| 93252_Secondary | 0.9 | 93579_CCD1106 | 20.9 |
| Th1/Th2/Tr1_anti-CD95 CH11 | | (Keratinocytes)_none | |
| 93103_LAK cells_resting | 15.8 | 93580_CCD1106 | 7.4 |
| | | (Keratinocytes)_TNFa and | |
| | | IFNg** | |
| 93788_LAK cells_IL-2 | 6.1 | 93791_Liver Cirrhosis | 3.7 |
| 93787_LAK cells_IL-2 + IL-12 | 8.8 | 93792_Lupus Kidney | 1.9 |
| 93789_LAK cells_IL-2 + IFN | 11.7 | 93577_NCI-H292 | 18.6 |
| gamma | | | |
| 93790_LAK cells_IL-2 + IL-18 | 13.2 | 93358_NCI-H292_IL-4 | 33.7 |
| 93104_LAK | 8.5 | 93360_NCI-H292_IL-9 | 36.9 |
| cells_PMA/ionomycin and IL-18 | | | |
| 93578_NK Cells IL-2_resting | 2.5 | 93359_NCI-H292_IL-13 | 20.0 |
| 93109_Mixed Lymphocyte | 17.0 | 93357_NCI-H292_IFN gamma | 20.4 |
| Reaction_Two Way MLR | | | |
| 93110_Mixed Lymphocyte | 10.2 | 93777_HPAEC_- | 18.8 |
| Reaction_Two Way MLR | | | |
| 93111_Mixed Lymphocyte | 7.4 | 93778_HPAEC_IL-1 beta/TNA | 18.9 |
| Reaction_Two Way MLR | | alpha | |
| 93112_Mononuclear Cells | 2.4 | 93254_Normal Human Lung | 9.5 |
| (PBMCs)_resting | | Fibroblast_none | |
| 93113_Mononuclear Cells | 23.7 | 93253_Normal Human Lung | 3.7 |
| (PBMCs)_PWM | | Fibroblast_TNFa (4 ng/ml) and IL- | |
| | | 1b (1 ng/ml) | |
| 93114_Mononuclear Cells | 9.6 | 93257_Normal Human Lung | 24.7 |
| (PBMCs)_PHA-L | | Fibroblast_IL-4 | |
| 93249_Ramos (B cell)_none | 30.4 | 93256_Normal Human Lung | 19.2 |
| | | Fibroblast_IL-9 | |
| 93250_Ramos (B | 100.0 | 93255_Normal Human Lung | 14.3 |
| cell)_ionomycin | | Fibroblast_IL-13 | |
| 93349_B lymphocytes_PWM | 70.2 | 93258_Normal Human Lung | 23.2 |
| | | Fibroblast_IFN gamma | |
| 93350_B lymphoytes_CD40L | 5.5 | 93106_Dermal Fibroblasts | 47.0 |
| and IL-4 | | CCD1070_resting | |
| 92665_EOL-1 | 11.7 | 93361_Dermal Fibroblasts | 42.3 |
| (Eosinophil)_dbcAMP | | CCD1070_TNF alpha 4 ng/ml | |
| differentiated | | | |
| 93248_EOL-1 | 6.3 | 93105_Dermal Fibroblasts | 20.2 |
| (Eosinophil)_dbcAMP/PMAiono- | | CCD1070_IL-beta 1 ng/ml | |
| mycin | | | |
| 93356_Dendritic Cells_none | 12.4 | 93772_dermal fibroblast_IFN | 9.2 |
| | | gamma | |
| 93355_Dendritic Cells_LPS 100 | 9.0 | 93771_dermal fibroblast_IL-4 | 22.1 |
| ng/ml | | | |
| 93775_Dendritic Cells_anti- | 12.5 | 93260_IBD Colitis 2 | 1.1 |
| CD40 | | | |
| 93774_Monocytes_resting | 15.2 | 93261_IBD Crohns | 1.0 |
| 93776_Monocytes_LPS 50 | 11.7 | 735010_Colon _normal | 4.1 |
| ng/ml | | | |
| 93581_Macrophages_resting | 41.8 | 735019_Lung_none | 8.9 |
| 93582_Macrophages_LPS 100 | 6.8 | 64028-1_Thymus_none | 10.5 |
| ng/ml | | | |
| 93098_HUVEC | 35.8 | 64030-1_Kidney_none | 3.6 |
| (Endothelial)_none | | | |
| 93099_HUVEC | 58.2 | | |
| (Endothelial)_starved | | | |

TABLE 43
Panel CNS_1

| | Relative Expression (%) | |
|---|---|---|
| | cns1x4tm6185t_{—} | cns1tm6569t_{—} |
| Tissue Name | ag2458_b2 | ag2458 |
| 102633_BA4 Control | 47.4 | 40.3 |
| 102641_BA4 Control2 | 74.3 | 47.3 |
| 102625_BA4 Alzheimer's2 | 16.6 | 5.6 |
| 102649_BA4 Parkinson's | 40.9 | 42.6 |
| 102656_BA4 Parkinson's2 | 100.0 | 74.7 |
| 102664_BA4 Huntington's | 41.3 | 33.2 |
| 102671_BA4 Huntington's2 | 18.3 | 6.2 |
| 102603_BA4 PSP | 8.4 | 13.1 |
| 102610_BA4 PSP2 | 35.5 | 40.9 |
| 102588_BA4 Depression | 26.1 | 21.6 |
| 102596_BA4 Depression2 | 6.0 | 3.0 |
| 102634_BA7 Control | 75.7 | 32.8 |
| 102642_BA7 Control2 | 75.9 | 46.0 |
| 102626_BA7 Alzheimer's2 | 6.1 | 2.2 |
| 102650_BA7 Parkinson's | 27.3 | 14.1 |
| 102657_BA7 Parkinson's2 | 30.2 | 40.3 |
| 102665_BA7 Huntington's | 43.5 | 37.4 |
| 102672_BA7 Huntington's2 | 44.5 | 27.9 |
| 102604_BA7 PSP | 47.9 | 55.9 |
| 102611_BA7 PSP2 | 23.5 | 20.0 |
| 102589_BA7 Depression | 20.7 | 18.7 |
| 102632_BA9 Control | 33.5 | 25.7 |
| 102640_BA9 Control2 | 81.2 | 72.2 |
| 102617_BA9 Alzheimer's | 5.1 | 13.9 |
| 102624_BA9 Alzheimer's2 | 29.0 | 15.6 |
| 102648_BA9 Parkinson's | 46.8 | 33.0 |
| 102655_BA9 Parkinson's2 | 61.1 | 95.9 |
| 102663_BA9 Huntington's | 67.5 | 100.0 |
| 102670_BA9 Huntington's2 | 20.5 | 12.9 |
| 102602_BA9 PSP | 9.5 | 18.6 |
| 102609_BA9 PSP2 | 3.6 | 12.5 |
| 102587_BA9 Depression | 10.4 | 10.2 |
| 102595_BA9 Depression2 | 5.4 | 11.3 |
| 102635_BA17 Control | 30.3 | 34.9 |
| 102643_BA17 Control2 | 45.0 | 30.8 |
| 102627_BA17 Alzheimer's2 | 12.9 | 4.3 |
| 102651_BA17 Parkinson's | 52.9 | 27.2 |
| 102658_BA17 Parkinson's2 | 55.5 | 37.4 |
| 102666_BA17 Huntington's | 34.0 | 24.3 |
| 102673_BA17 Huntington's2 | 13.7 | 8.2 |
| 102590_BA17 Depression | 17.3 | 20.9 |
| 102597_BA17 Depression2 | 38.8 | 13.5 |
| 102605_BA17 PSP | 38.0 | 38.7 |
| 102612_BA17 PSP2 | 12.4 | 13.3 |
| 102637_Sub Nigra Control | 42.0 | 24.3 |
| 102645_Sub Nigra Control2 | 41.1 | 97.3 |
| 102629_Sub Nigra Alzheimer's2 | 19.4 | 7.6 |
| 102660_Sub Nigra Parkinson's2 | 85.7 | 28.7 |
| 102667_Sub Nigra Huntington's | 53.9 | 28.9 |
| 102674_Sub Nigra Huntington's2 | 41.8 | 25.2 |
| 102614_Sub Nigra PSP2 | 9.8 | 6.3 |
| 102592_Sub Nigra Depression | 11.7 | 6.9 |
| 102599_Sub Nigra Depression2 | 5.8 | 10.6 |
| 102636_Glob Palladus Control | 17.5 | 19.1 |
| 102644_Glob Palladus Control2 | 16.3 | 8.5 |
| 102620_Glob Palladus Alzheimer's | 11.9 | 12.4 |
| 102628_Glob Palladus Alzheimer's2 | 7.3 | 10.8 |
| 102652_Glob Palladus Parkinson's | 84.3 | 59.0 |
| 102659_Glob Palladus Parkinson's2 | 22.3 | 11.3 |
| 102606_Glob Palladus PSP | 10.6 | 7.1 |
| 102613_Glob Palladus PSP2 | 15.7 | 9.2 |
| 102591_Glob Palladus Depression | 9.2 | 3.7 |
| 102638_Temp Pole Control | 22.1 | 17.8 |
| 102646_Temp Pole Control2 | 45.1 | 51.0 |
| 102622_Temp Pole Alzheimer's | 11.4 | 14.1 |
| 102630_Temp Pole Alzheimer's2 | 7.2 | 14.1 |
| 102653_Temp Pole Parkinson's | 25.9 | 22.4 |
| 102661_Temp Pole Parkinson's2 | 25.4 | 39.8 |
| 102668_Temp Pole Huntington's | 39.1 | 37.4 |
| 102607_Temp Pole PSP | 13.7 | 6.8 |
| 102615_Temp Pole PSP2 | 17.0 | 7.9 |
| 102600_Temp Pole Depression2 | 3.1 | 15.5 |
| 102639_Cing Gyr Control | 58.5 | 53.6 |
| 102647_Cing Gyr Control2 | 38.5 | 47.0 |
| 102623_Cing Gyr Alzheimer's | 17.0 | 48.6 |
| 102631_Cing Gyr Alzheimer's2 | 11.7 | 8.3 |
| 102654_Cing Gyr Parkinson's | 31.0 | 43.2 |
| 102662_Cing Gyr Parkinson's2 | 41.7 | 48.0 |
| 102669_Cing Gyr Huntington's | 84.9 | 77.9 |
| 102676_Cing Gyr Huntington's2 | 22.1 | 7.2 |
| 102608_Cing Gyr PSP | 26.1 | 19.6 |
| 102616_Cing Gyr PSP2 | 8.9 | 11.3 |
| 102594_Cing Gyr Depression | 12.7 | 11.5 |
| 102601_Cing Gyr Depression2 | 8.5 | 10.8 |

TABLE 44
Panel CNS_1.1

| | Relative Expression (%) | |
|---|---|---|
| | cns_{—} | cns_{—} |
| | 1.1tm6733t_{—} | 1.1tm6734t_{—} |
| Tissue Name | ag2458_b2 | ag2458_b2 |
| 102601_Cing Gyr Depression2 | 8.4 | 9.7 |
| 102594_Cing Gyr Depression | 11.0 | 18.8 |
| 102616_Cing Gyr PSP2 | 7.7 | 7.4 |
| 102608_Cing Gyr PSP | 22.2 | 23.2 |
| 102676_Cing Gyr Huntington's2 | 9.1 | 14.5 |
| 102669_Cing Gyr Huntington's | 44.5 | 71.0 |
| 102662_Cing Gyr Parkinson's2 | 32.6 | 39.6 |
| 102654_Cing Gyr Parkinson's | 36.9 | 49.2 |
| 102631_Cing Gyr Alzheimer's2 | 14.1 | 18.0 |
| 102623_Cing Gyr Alzheimer's | 30.0 | 11.7 |
| 102647_Cing Gyr Control2 | 26.2 | 39.0 |
| 102639_Cing Gyr Control | 47.8 | 75.4 |
| 102600_Temp Pole Depression2 | 8.3 | 7.6 |
| 102615_Temp Pole PSP2 | 4.5 | 4.4 |
| 102607_Temp Pole PSP | 5.3 | 5.9 |
| 102668_Temp Pole Huntington's | 34.4 | 46.0 |
| 102661_Temp Pole Parkinson's2 | 18.2 | 49.8 |
| 102653_Temp Pole Parkinson's | 37.9 | 37.0 |
| 102630_Temp Pole Alzheimer's2 | 7.4 | 6.2 |
| 102622_Temp Pole Alzheimer's | 3.1 | 7.1 |
| 102646_Temp Pole Control2 | 32.7 | 47.8 |
| 102638_Temp Pole Control | 13.2 | 13.4 |
| 102591_Glob Palladus Depression | 8.6 | 7.3 |
| 102613_Glob Palladus PSP2 | 12.8 | 7.7 |
| 102606_Glob Palladus PSP | 17.2 | 2.1 |
| 102659_Glob Palladus Parkinson's2 | 24.3 | 34.4 |
| 102652_Glob Palladus Parkinson's | 64.2 | 87.9 |
| 102628_Glob Palladus Alzheimer's2 | 9.5 | 6.3 |
| 102620_Glob Palladus Alzheimer's | 17.8 | 14.1 |
| 102644_Glob Palladus Control2 | 16.2 | 14.2 |
| 102636_Glob Palladus Control | 8.3 | 23.7 |
| 102599_Sub Nigra Depression2 | 3.9 | 6.5 |
| 102592_Sub Nigra Depression | 33.4 | 0.0 |
| 102614_Sub Nigra PSP2 | 13.1 | 11.0 |
| 102674_Sub Nigra Huntington's2 | 48.1 | 46.0 |
| 102667_Sub Nigra Huntington's | 42.8 | 59.7 |
| 102660_Sub Nigra Parkinson's2 | 55.9 | 78.3 |
| 102629_Sub Nigra Alzheimer's2 | 14.1 | 18.1 |
| 102645_Sub Nigra Control2 | 18.8 | 14.3 |
| 102637_Sub Nigra Control | 32.0 | 39.4 |
| 102597_BA17 Depression2 | 29.8 | 33.3 |
| 102590_BA17 Depression | 16.9 | 22.2 |
| 102612_BA17 PSP2 | 10.6 | 5.7 |
| 102605_BA17 PSP | 34.9 | 40.3 |
| 102673_BA17 Huntington's2 | 10.4 | 19.3 |
| 102666_BA17 Huntington's | 51.2 | 63.3 |
| 102658_BA17 Parkinson's2 | 62.4 | 92.4 |
| 102651_BA17 Parkinson's | 40.2 | 51.9 |
| 102627_BA17 Alzheimer's2 | 3.3 | 9.4 |
| 102643_BA17 Control2 | 71.2 | 69.4 |
| 102635_BA17 Control | 42.0 | 43.4 |
| 102595_BA9 Depression2 | 7.6 | 16.4 |
| 102587_BA9 Depression | 8.1 | 19.9 |
| 102609_BA9 PSP2 | 2.7 | 11.7 |
| 102602_BA9 PSP | 14.2 | 21.9 |
| 102670_BA9 Huntington's2 | 16.1 | 21.8 |
| 102663_BA9 Huntington's | 59.1 | 37.4 |
| 102655_BA9 Parkinson's2 | 56.5 | 76.5 |
| 102648_BA9 Parkinson's | 34.0 | 48.2 |
| 102624_BA9 Alzheimer's2 | 30.7 | 20.4 |
| 102617_BA9 Alzheimer's | 12.8 | 2.1 |
| 102640_BA9 Control2 | 67.2 | 100.0 |
| 102632_BA9 Control | 24.3 | 44.1 |
| 102589_BA7 Depression | 19.9 | 26.2 |
| 102611_BA7 PSP2 | 38.5 | 36.8 |
| 102604_BA7 PSP | 49.7 | 56.2 |
| 102672_BA7 Huntington's2 | 29.3 | 49.0 |
| 102665_BA7 Huntington's | 34.6 | 45.8 |
| 102657_BA7 Parkinson's2 | 25.4 | 43.4 |
| 102650_BA7 Parkinson's | 28.9 | 23.1 |
| 102626_BA7 Alzheimer's2 | 11.1 | 9.2 |
| 102642_BA7 Control2 | 41.8 | 38.8 |
| 102634_BA7 Control | 55.4 | 45.2 |
| 102596_BA4 Depression2 | 6.4 | 19.6 |
| 102588_BA4 Depression | 0.0 | 23.8 |
| 102610_BA4 PSP2 | 45.8 | 23.0 |
| 102603_BA4 PSP | 13.0 | 23.9 |
| 102671_BA4 Huntington's2 | 6.0 | 10.7 |
| 102664_BA4 Huntington's | 31.2 | 23.6 |
| 102656_BA4 Parkinson's2 | 100.0 | 93.1 |
| 102649_BA4 Parkinson's | 58.9 | 55.2 |
| 102625_BA4 Alzheimer's | 8.2 | 2.2 |
| 102641_BA4 Control2 | 39.1 | 0.0 |
| 102633_BA4 Control | 43.8 | 51.1 |

TABLE 45
Panel CNS_Neurodegeneration_v1.0

| | Relative | | Relative |
|---|---|---|---|
| | Expression (%) | | Expression (%) |
| | tm7017t_{—} | | tm7017t_{—} |
| Tissue Name | ag2458_b2_s2 | Tissue Name | ag2458_b2_s2 |
| AD 1 Hippo | 2.8 | Control (Path) 3 Temporal Ctx | 1.5 |
| AD 2 Hippo | 6.2 | Control (Path) 4 Temporal Ctx | 11.6 |
| AD 3 Hippo | 1.1 | AD 1 Occipital Ctx | 3.4 |
| AD 4 Hippo | 1.6 | AD 2 Occipital Ctx (Missing) | 0.0 |
| AD 5 hippo | 59.9 | AD 3 Occipital Ctx | 1.1 |
| AD 6 Hippo | 12.9 | AD 4 Occipital Ctx | 6.3 |
| Control 2 Hippo | 7.2 | AD 5 Occipital Ctx | 17.5 |
| Control 4 Hippo | 2.2 | AD 6 Occipital Ctx | 38.1 |
| Control (Path) 3 Hippo | 1.0 | Control 1 Occipital Ctx | 0.8 |
| AD 1 Temporal Ctx | 2.4 | Control 2 Occipital Ctx | 16.6 |
| AD 2 Temporal Ctx | 9.8 | Control 3 Occipital Ctx | 4.2 |
| AD 3 Temporal Ctx | 1.1 | Control 4 Occipital Ctx | 1.5 |
| AD 4 Temporal Ctx | 5.5 | Control (Path) 1 Occipital Ctx | 22.9 |
| AD 5 Inf Temporal Ctx | 100.0 | Control (Path) 2 Occipital Ctx | 3.5 |
| AD 5 SupTemporal Ctx | 46.5 | Control (Path) 3 Occipital Ctx | 0.3 |
| AD 6 Inf Temporal Ctx | 14.2 | Control (Path) 4 Occipital Ctx | 3.6 |
| AD 6 Sup Temporal Ctx | 17.9 | Control 1 Parietal | 1.9 |
| Control 1 Temporal Ctx | 1.9 | Control 2 Parietal | 27.9 |
| Control 2 Temporal Ctx | 13.6 | Control 3 Parietal | 7.8 |
| Control 3 Temporal Ctx | 5.7 | Control (Path) 1 Parietal | 24.8 |
| Control 4 Temporal Ctx | 2.3 | Control (Path) 2 Parietal | 8.9 |
| Control (Path) 1 Temporal Ctx | 16.7 | Control (Path) 3 Parietal | 1.2 |
| Control (Path) 2 Temporal Ctx | 9.4 | Control (Path) 4 Parietal | 16.1 |

Panel 1.3D Summary: Ag2458 Results from two experiments using the same probe and primer sets are in very good agreement. Highest expression is seen in breast cancer in both runs (CT=28-30). The NOV6A gene is expressed at moderate levels across a wide variety of cancerous cell lines as opposed to normal tissues. Thus, the expression of this gene could be used to distinguish cell line derived samples from normal tissue derived samples. In addition, since the cell lines are derived from cancerous tissue, expression of the NOV6A gene potentially could be used to distinguish cancerous material from normal material and specifically, as a marker for breast cancer. Finally, since the expression of this gene is largely associated with cancerous cells, therapeutic modulation of this gene product, through the use of small molecule drugs or antibodies, might be beneficial in the treatment of breast or other cancers.

Panel 2D Summary: Ag2458 In this experiment, expression of the NOV6A gene is most pronounced in lung cancer with a CT of 28.4. Other tissues also demonstrating significant expression are ocular melanoma (CT=28.8), bladder cancer (CT=29.0), ovarian cancer and gastric cancers. The NOV6A gene appears to show a stronger association with malignant tissue as compared to normal adjacent tissue. For instance, there is at least a 2 to 3 fold difference in expression level between malignant tissue and normal adjacent tissue samples derived from gastric, ovary, lung and colon cancers. Thus, the NOV6A gene could be used to distinguish between malignant and normal tissues of the stomach, ovary, lung and colon. In addition, therapeutic modulation of this gene product, through the use of small molecule drugs or antibodies might be of benefit in the treatment of the associated cancers.

Panel 3D Summary: Ag2458 The NOV6A gene is highly expressed in lung cancer (CT=27.4) and expressed at moderate/low level among all the tissue samples in the panel. Please see panel 2D for a discussion of potential utility for this expression profile.

Panel 4D Summary: Ag2458 The NOV6A gene is highly expressed in an activated B cell line, Ramos (26.8) and in primary B cells activated by PWM(27.3). The gene is also expressed at moderate/low levels among most of the tissues in the sample regardless of treatment.

Since the NOV6A gene most probably encodes a neurolysin like molecule with potential enzymatic activity, it may be important in maintaining normal cellular functions in a number of tissues. Therapies designed with the protein encoded by the NOV6A gene could be important in regulating cellular viability or function.

Panel CNS_{—}1 Summary: Ag2458 Results from two experiments using the same probe/primer set are in good agreement. Highest expression of the NOV6A gene occurs at moderate levels (CT=30.7) in Brodman's Area 4 from a Parkinson's patient and Brodman's Area 9 from a Huntington's patient (CT=30.2). This gene is expressed at moderate/low levels across most of the tissues (healthy and diseased) in the sample. Please see panel CNS_neurodegeneration_v1.0 for potential utility of this gene in diseases of the CNS.

Panel CNS_{—}1.1 Summary: Ag2458 Results from two experiments using the same probe/primer set are in very good agreement. Highest expression of the NOV6A gene occurs in Brodman's Area 4 in a Parkinson's patient (CT=31.6) and Brodman's Area 9 in a control patient (CT=32.2). Please see panel CNS_neurodegeneration_V1.0 for a discussion of potential utility of this gene in diseases of the CNS.

Panel CNS_Neurodegeneration_v1.0 Summary Ag2458 The NOV6A gene is highly expressed in the a tissue sample from the inferior temporal cortex from an Alzheimer's patient (CT=27.6) and expressed at moderate levels in samples from the occipital cortex (CT=29), superior temporal cortex (CT=28.7), and the hippocampus (CT=28.4) of an Alzheimer's patien. Significant expression is also detected in tissue samples derived from a control patient originating in the parietal region (CT=29.6), and occipital cortex (CT=29.7) regions of the brain. Expression of this gene is detectable at moderate/low levels in most of the tissues in this sample. The wide expression of the gene across many tissues involved in the central nervous system indicates that the NOV6A gene, which encodes a neurolysin-like molecule with enzymatic activity, has specific function and utility to CNS processes. Aminopeptidases are increased in Huntington's disease, and mediate neurotoxic processing of A-beta in Alzheimer's disease brains, indicating that agents that inhibit the activity of these enzymes may be useful in treating neurodegenerative disorders, including Alzheimer's disease and Huntington's disease. Metallopeptidases have been implicated in the normal and disease-state processing of peptides involved in neurological, endocrine and cardiovascular functions. In this context, specific inhibitors of these enzymes could selectively modulate peptide levels and thus have considerable therapeutic potential for the treatment of stroke, epilepsy, schizophrenia and depression. Thus, therapeutic modulation of the protein encoded by the NOV6A gene, may have considerable efficacy in treating these central nervous system disorders. (Shrimpton and Smith, J Pept Sci 6:251-63, 2000; Saido, Neurobiol Aging 19:S69-75, 1998; Kaneko et al., Neuroscience 104:1003-11, 2001; Mantle et al., J Neurol Sci. 131:65-70, 1995.)

NOV7a: Gamma-aminobutyric Acid (GABA) Transporter-like

Expression of the NOV7a gene (ba122o1) was assessed using the primer-probe sets Ag1481 and Ag2307 described in Tables 46 and 47. Results of the RTQ-PCR runs are shown in Tables 48, 49, 50, 51, 52, 53, and 54.

TABLE 46

Probe Name Ag1481

| | | | | | |
|---|---|---|---|---|---|
| | | | | Start | |
| Primers | Sequences | TM | Length | Position | SEQ ID NO: |

| | | | | | |
|---|---|---|---|---|---|
| Forward | 5′-TGGGAGAAGGTCAAGTTCTACA-3′ | 58.8 | 22 | 1020 | 170 |
| Probe | FAM-5′-ATCTCCATTGGCATCATCGTGTTCAG- | 69.2 | 26 | 1062 | 171 |
| | 3′-TAMRA | | | | |
| Reverse | 5′-GCAGGAAGATCTGAGACGTGTA-3′ | 59.5 | 22 | 1089 | 172 |

TABLE 47

Probe Name Ag23O7

| | | | | | |
|---|---|---|---|---|---|
| | | | | Start | |
| Primers | Sequences | TM | Length | Position | SEQ ID NO: |

| | | | | | |
|---|---|---|---|---|---|
| Forward | 5′-GGAACTTCTTGACGTCGATGTA-3′ | 59.2 | 22 | 705 | 173 |
| Probe | TET-5′-AACTTGACCTTCTCCCAGGCCCAGT- | 70 | 25 | 728 | 174 |
| | 3′-TAMRA | | | | |
| Reverse | 5′-TCGTCATCAATATCCTGGTCAT-3′ | 59.3 | 22 | 779 | 175 |

TABLE 48
Panel 1.3D

| | | | Relative |
|---|---|---|---|
| | Relative Expression (%) | | Expression (%) |
| | 1.3dtm4170f_{—} | 1.3dx4tm5350f_{—} | 1.3dtm4557t_{—} |
| Tissue Name | ag1481 | ag1481_a2 | ag2307 |
| Liver adenocarcinoma | 0.0 | 0.0 | 0.0 |
| Pancreas | 0.0 | 0.0 | 0.0 |
| Pancreatic ca. CAPAN 2 | 0.0 | 0.0 | 0.0 |
| Adrenal gland | 0.0 | 2.1 | 0.0 |
| Thyroid | 0.0 | 0.0 | 0.0 |
| Salivary gland | 0.0 | 0.0 | 0.0 |
| Pituitary gland | 13.8 | 17.8 | 4.5 |
| Brain (fetal) | 9.0 | 23.8 | 3.0 |
| Brain (whole) | 24.7 | 100.0 | 22.7 |
| Brain (amygdala) | 25.9 | 48.7 | 23.5 |
| Brain (cerebellum) | 12.2 | 60.6 | 15.6 |
| Brain (hippocampus) | 100.0 | 67.2 | 27.2 |
| Brain (substantia nigra) | 4.5 | 23.0 | 3.8 |
| Brain (thalamus) | 24.8 | 63.6 | 21.6 |
| Cerebral Cortex | 52.8 | 70.9 | 100.0 |
| Spinal cord | 1.8 | 10.1 | 4.3 |
| CNS ca. (glio/astro) U87-MG | 0.0 | 0.0 | 0.0 |
| CNS ca. (glio/astro) U-118-MG | 0.0 | 0.0 | 0.0 |
| CNS ca. (astro) SW1783 | 0.0 | 0.0 | 0.0 |
| CNS ca.* (neuro; met) SK-N-AS | 0.1 | 0.0 | 0.0 |
| CNS ca. (astro) SF-539 | 0.0 | 0.0 | 0.0 |
| CNS ca. (astro) SNB-75 | 0.0 | 0.0 | 0.0 |
| CNS ca. (glio) SNB-19 | 0.0 | 0.0 | 0.0 |
| CNS ca. (glio) U251 | 0.0 | 0.3 | 0.0 |
| CNS ca. (glio) SF-295 | 0.0 | 0.0 | 0.0 |
| Heart (fetal) | 0.0 | 0.5 | 0.0 |
| Heart | 0.0 | 0.0 | 0.0 |
| Fetal Skeletal | 0.2 | 4.1 | 0.3 |
| Skeletal muscle | 0.0 | 0.0 | 0.0 |
| Bone marrow | 0.0 | 0.0 | 0.0 |
| Thymus | 0.0 | 0.0 | 0.1 |
| Spleen | 0.0 | 1.0 | 0.0 |
| Lymph node | 0.1 | 0.0 | 0.0 |
| Colorectal | 0.4 | 0.0 | 0.0 |
| Stomach | 0.0 | 0.0 | 0.0 |
| Small intestine | 0.0 | 0.5 | 0.0 |
| Colon ca. SW480 | 0.0 | 0.0 | 0.0 |
| Colon ca.* (SW480 met) SW620 | 0.0 | 0.0 | 0.0 |
| Colon ca. HT29 | 0.0 | 0.0 | 0.0 |
| Colon ca. HCT-116 | 0.0 | 1.0 | 0.0 |
| Colon ca. CaCo-2 | 0.0 | 0.0 | 0.0 |
| 83219 CC Well to Mod Diff | 0.0 | 0.0 | 0.0 |
| (ODO3866) | | | |
| Colon ca. HCC-2998 | 0.0 | 0.0 | 0.0 |
| Gastric ca.* (liver met) NCI-N87 | 0.0 | 0.0 | 0.0 |
| Bladder | 0.0 | 0.0 | 0.0 |
| Trachea | 0.0 | 0.0 | 0.1 |
| Kidney | 0.0 | 0.0 | 0.0 |
| Kidney (fetal) | 0.8 | 3.5 | 0.2 |
| Renal ca. 786-0 | 0.0 | 0.0 | 0.0 |
| Renal ca. A498 | 0.0 | 0.0 | 0.0 |
| Renal ca. RXF 393 | 0.0 | 0.0 | 0.0 |
| Renal ca. ACHN | 0.0 | 0.0 | 0.0 |
| Renal ca. UO-31 | 0.0 | 0.0 | 0.0 |
| Renal ca. TK-10 | 0.0 | 0.0 | 0.0 |
| Liver | 0.0 | 0.0 | 0.0 |
| Liver (fetal) | 0.0 | 0.0 | 0.0 |
| Liver ca. (hepatoblast) HepG2 | 0.0 | 0.0 | 0.0 |
| Lung | 0.0 | 0.0 | 0.0 |
| Lung (fetal) | 0.0 | 0.2 | 0.0 |
| Lung ca. (small cell) LX-1 | 0.0 | 0.0 | 0.0 |
| Lung ca. (small cell) NCI-H69 | 0.0 | 0.0 | 0.0 |
| Lung ca. (s.cell var.) SHP-77 | 0.0 | 0.0 | 0.0 |
| Lung ca. (large cell)NCI-H460 | 0.0 | 1.7 | 0.0 |
| Lung ca. (non-sm. cell) A549 | 0.0 | 0.0 | 0.0 |
| Lung ca. (non-s.cell) NCI-H23 | 0.0 | 0.0 | 0.0 |
| Lung ca (non-s.cell) HOP-62 | 0.0 | 0.0 | 0.0 |
| Lung ca. (non-s.cl) NCI-H522 | 9.4 | 14.4 | 8.4 |
| Lung ca. (squam.) SW 900 | 0.0 | 0.0 | 0.0 |
| Lung ca. (squam.) NCI-H596 | 0.0 | 0.0 | 0.0 |
| Mammary gland | 0.0 | 1.6 | 0.0 |
| Breast ca.* (pl. effusion) MCF-7 | 0.0 | 0.0 | 0.0 |
| Breast ca.* (pl.ef) MDA-MB-231 | 0.0 | 0.0 | 0.0 |
| Breast ca.* (pl. effusion) T47D | 0.0 | 0.0 | 0.0 |
| Breast ca. BT-549 | 0.0 | 0.0 | 0.0 |
| Breast ca. MDA-N | 0.0 | 0.0 | 0.3 |
| Ovary | 0.4 | 0.0 | 0.2 |
| Ovarian ca. OVCAR-3 | 0.0 | 0.0 | 0.0 |
| Ovarian ca. OVCAR-4 | 0.0 | 0.0 | 0.0 |
| Ovarian ca. OVCAR-5 | 0.0 | 0.0 | 0.0 |
| Ovarian ca. OVCAR-8 | 0.0 | 0.0 | 0.0 |
| Ovarian ca. IGROV-1 | 0.1 | 0.0 | 0.0 |
| Ovarian ca.* (ascites) SK-OV-3 | 0.0 | 0.0 | 0.0 |
| Uterus | 0.0 | 0.0 | 0.0 |
| Placenta | 0.0 | 0.4 | 0.0 |
| Prostate | 0.0 | 0.0 | 0.0 |
| Prostate ca.* (bone met)PC-3 | 0.0 | 0.0 | 0.0 |
| Testis | 1.8 | 2.2 | 0.9 |
| Melanoma Hs688(A).T | 0.0 | 0.0 | 0.0 |
| Melanoma* (met) Hs688(B).T | 0.0 | 0.0 | 0.0 |
| Melanoma UACC-62 | 0.3 | 0.0 | 0.0 |
| Melanoma M14 | 0.0 | 0.0 | 0.0 |
| Melanoma LOXIMVI | 0.0 | 0.0 | 0.0 |
| Melanoma* (met) SK-MEL-5 | 0.0 | 0.0 | 0.0 |
| Adipose | 0.0 | 0.0 | 0.1 |

TABLE 49
Panel General_screening_panel_v1.4

| | Relative Expression (%) | |
|---|---|---|
| | General_screening_{—} | |
| | panel_v1.4(384) | |
| | tm6981f_{—} | tm7150f_ag1481_{—} |
| Tissue Name | ag1481_b2 | b1 |
| D6005-01_Human adipose | 0.0 | 0.1 |
| 112193_Metastatic melanoma | 0.0 | 0.0 |
| 112192_Metastatic melanoma | 0.0 | 0.0 |
| 95280_Epidermis (metastatic melanoma) | 0.0 | 0.0 |
| 95279_Epidermis (metastatic melanoma) | 0.0 | 0.0 |
| Melanoma (met)_SK-MEL-5 | 0.3 | 0.0 |
| 112196_Tongue (oncology) | 0.0 | 0.0 |
| 113461_Testis Pool | 1.6 | 0.2 |
| Prostate ca. (bone met)_PC-3 | 0.0 | 0.0 |
| 113455_Prostate Pool | 0.0 | 0.0 |
| 103396_Placenta | 0.0 | 0.0 |
| 113463_Uterus Pool | 0.0 | 0.0 |
| Ovarian carcinoma_OVCAR-3 | 0.0 | 0.2 |
| Ovarian carcinoma(ascites)_SK-OV-3 | 0.0 | 0.1 |
| 95297_Adenocarcinoma (ovary) | 0.0 | 0.0 |
| Ovarian carcinoma_OVCAR-5 | 0.0 | 0.2 |
| Ovarian carcinoma_IGROV-1 | 0.0 | 0.0 |
| Ovarian carcinoma_OVCAR-8 | 0.0 | 0.0 |
| 103368_Ovary | 0.0 | 0.0 |
| MCF7_breast carcinoma(pleural effusion) | 0.0 | 0.0 |
| Breast ca. (pleural effusion)_MDA-MB-231 | 0.1 | 0.0 |
| 112189_ductal cell carcinoma(breast) | 0.0 | 0.0 |
| Breast ca. (pleural effusion)_T47D | 0.2 | 0.0 |
| Breast carcinoma_MDA-N | 0.0 | 0.0 |
| 113452_Breast Pool | 0.0 | 0.0 |
| 103398_Trachea | 0.4 | 0.0 |
| 112354_lung | 0.0 | 0.0 |
| 103374_Fetal Lung | 0.6 | 0.0 |
| 94921_Small cell carcinoma of the lung | 0.3 | 1.1 |
| Lung ca. (small cell)_LX-1 | 0.0 | 0.0 |
| 94919_Small cell carcinoma of the lung | 0.2 | 0.3 |
| Lung ca. (s.cell var.)_SHP-77 | 0.0 | 0.0 |
| 95268_Lung (Large cell carcinoma) | 0.0 | 0.0 |
| 94920_Small cell carcinoma of the lung | 0.0 | 0.0 |
| Lung ca. (non-s.cell)_NCI-H23 | 0.0 | 0.5 |
| Lung ca. (large cell)_NCI-H460 | 0.0 | 0.0 |
| Lung ca. (non-s.cell)_HOP-62 | 0.0 | 0.0 |
| Lung ca. (non-s.cl)_NCI-H522 | 62.3 | 43.4 |
| 103392_Liver | 0.0 | 0.0 |
| 103393_Fetal Liver | 0.0 | 0.0 |
| Liver ca. (hepatoblast)_HepG2 | 0.0 | 0.0 |
| 113465_Kidney Pool | 0.2 | 0.0 |
| 103373_Fetal Kidney | 2.7 | 1.6 |
| Renal ca._786-0 | 0.0 | 0.0 |
| 112188_renal cell carcinoma | 0.0 | 0.0 |
| Renal ca._ACHN | 0.0 | 0.1 |
| 112190_Renal cell carcinoma | 0.0 | 0.0 |
| Renal ca._TK-10 | 0.0 | 0.1 |
| Bladder | 0.0 | 0.0 |
| Gastric ca. (liver met)_NCI-N87 | 0.0 | 0.2 |
| 112197_Stomach | 0.0 | 0.1 |
| 94938_Colon Adenocarcinoma | 0.0 | 0.0 |
| Colon ca._SW480 | 0.0 | 0.0 |
| Colon ca. (SW480 met)_SW620 | 0.0 | 0.0 |
| Colon ca._HT29 | 0.0 | 0.0 |
| Colon ca._HCT-116 | 0.3 | 0.4 |
| Colon ca._CaCo-2 | 0.0 | 0.0 |
| 83219_CC Well to Mod Diff (ODO3866) | 0.0 | 0.0 |
| 94936_Colon Adenocarcinoma | 0.0 | 0.0 |
| 94930_Colon | 0.0 | 0.0 |
| 94935_Colon Adenocarcinoma | 0.0 | 0.0 |
| 113468_Colon Pool | 0.2 | 0.0 |
| 113457_Small Intestine Pool | 0.2 | 0.0 |
| 113460_Stomach Pool | 0.0 | 0.0 |
| 113467_Bone Marrow Pool | 0.0 | 0.0 |
| 103371_Fetal Heart | 0.0 | 0.0 |
| 113451_Heart Pool | 0.1 | 0.0 |
| 113466_Lymph Node Pool | 0.2 | 0.0 |
| 103372_Fetal Skeletal Muscle | 0.0 | 0.0 |
| 113456_Skeletal Muscle Pool | 0.0 | 0.0 |
| 113459_Spleen Pool | 0.0 | 0.0 |
| 113462_Thymus Pool | 0.3 | 0.0 |
| CNS ca. (glio/astro)_U87-MG | 0.0 | 0.0 |
| CNS ca. (glio/astro)_U-118-MG | 0.0 | 0.0 |
| CNS ca. (neuro;met)_SK-N-AS | 0.2 | 0.0 |
| 95264_Brain astrocytoma | 0.0 | 0.0 |
| CNS ca. (astro)_SNB-75 | 0.0 | 0.0 |
| CNS ca. (glio)_SNB-19 | 0.0 | 0.0 |
| CNS ca. (glio)_SF-295 | 0.0 | 0.0 |
| 113447_Brain (Amygdala) Pool | 22.9 | 28.2 |
| 103382_Brain (cerebellum) | 100.0 | 100.0 |
| 64019-1_brain(fetal) | 28.7 | 35.7 |
| 113448_Brain (Hippocampus) Pool | 35.9 | 28.7 |
| 113464_Cerebral Cortex Pool | 28.5 | 33.0 |
| 113449_Brain (Substantia nigra) Pool | 25.6 | 53.9 |
| 113450_Brain (Thalamus) Pool | 14.1 | 30.2 |
| 103384_Brain (whole) | 11.7 | 47.2 |
| 113458_Spinal Cord Pool | 6.4 | 10.6 |
| 103375_Adrenal Gland | 0.0 | 0.0 |
| 113454_Pituitary gland Pool | 9.9 | 8.0 |
| 103397_Salivary Gland | 0.0 | 0.0 |
| 103369_Thyroid (female) | 0.0 | 0.0 |
| Pancreatic ca._CAPAN2 | 0.0 | 0.0 |
| 113453_Pancreas Pool | 0.0 | 0.0 |

Panel 50
Panel 2D

| | Relative Expression (%) | |
|---|---|---|
| | 2dtm4171f_{—} | 2dx4tm4724f_{—} |
| Tissue Name | ag1481 | ag1481_a1 |
| Normal Colon GENPAK 061003 | 0.0 | 0.0 |
| 83219 CC Well to Mod Diff (ODO3866) | 0.0 | 30.2 |
| 83220 CC NAT (ODO3866) | 0.0 | 0.0 |
| 83221 CC Gr.2 rectosigmoid (ODO3868) | 0.0 | 0.0 |
| 83222 CC NAT (ODO3868) | 0.0 | 0.0 |
| 83235 CC Mod Diff (ODO3920) | 0.0 | 0.0 |
| 83236 CC NAT (ODO3920) | 0.0 | 0.0 |
| 83237 CC Gr.2 ascend colon (ODO3921) | 50.7 | 50.0 |
| 83238 CC NAT (ODO3921) | 100.0 | 8.1 |
| 83241 CC from Partial Hepatectomy | 0.0 | 0.0 |
| (ODO4309) | | |
| 83242 Liver NAT (ODO4309) | 0.0 | 0.0 |
| 87472 Colon mets to lung (OD04451-01) | 0.0 | 0.0 |
| 87473 Lung NAT (OD04451-02) | 0.0 | 5.3 |
| Normal Prostate Clontech A + 6546-1 | 0.0 | 0.0 |
| 84140 Prostate Cancer (OD04410) | 0.0 | 0.0 |
| 84141 Prostate NAT (OD04410) | 0.0 | 0.0 |
| 87073 Prostate Cancer (OD04720-01) | 0.0 | 0.0 |
| 87074 Prostate NAT (OD04720-02) | 0.0 | 0.0 |
| Normal Lung GENPAK 061010 | 0.0 | 0.0 |
| 83239 Lung Met to Muscle (ODO4286) | 60.3 | 0.0 |
| 83240 Muscle NAT (ODO4286) | 0.0 | 0.0 |
| 84136 Lung Malignant Cancer (OD03126) | 0.0 | 0.0 |
| 84137 Lung NAT (OD03126) | 0.0 | 0.0 |
| 84871 Lung Cancer (OD04404) | 0.0 | 23.5 |
| 84872 Lung NAT (OD04404) | 0.0 | 100.0 |
| 84875 Lung Cancer (OD04565) | 0.0 | 0.0 |
| 84876 Lung NAT (OD04565) | 0.0 | 0.0 |
| 85950 Lung Cancer (OD04237-01) | 0.0 | 0.0 |
| 85970 Lung NAT (OD04237-02) | 0.0 | 0.0 |
| 83255 Ocular Mel Met to Liver | 0.0 | 0.0 |
| (ODO4310) | | |
| 83256 Liver NAT (ODO4310) | 0.0 | 0.0 |
| 84139 Melanoma Mets to Lung | 0.0 | 0.0 |
| (OD04321) | | |
| 84138 Lung NAT (OD04321) | 0.0 | 0.0 |
| Normal Kidney GENPAK 061008 | 0.0 | 0.0 |
| 83786 Kidney Ca, Nuclear grade 2 | 0.0 | 0.0 |
| (OD04338) | | |
| 83787 Kidney NAT (OD04338) | 0.0 | 0.0 |
| 83788 Kidney Ca Nuclear grade 1/2 | 0.0 | 0.0 |
| (OD04339) | | |
| 83789 Kidney NAT (OD04339) | 0.0 | 0.0 |
| 83790 Kidney Ca, Clear cell type | 0.0 | 0.0 |
| (OD04340) | | |
| 83791 Kidney NAT (OD04340) | 0.0 | 0.0 |
| 83792 Kidney Ca, Nuclear grade 3 | 22.5 | 0.0 |
| (OD04348) | | |
| 83793 Kidney NAT (OD04348) | 0.0 | 0.0 |
| 87474 Kidney Cancer (OD04622-01) | 39.8 | 0.0 |
| 87475 Kidney NAT (OD04622-03) | 0.0 | 0.0 |
| 85973 Kidney Cancer (OD04450-01) | 0.0 | 0.0 |
| 85974 Kidney NAT (OD04450-03) | 26.4 | 0.0 |
| Kidney Cancer Clontech 8120607 | 0.0 | 0.0 |
| Kidney NAT Clontech 8120608 | 0.0 | 0.0 |
| Kidney Cancer Clontech 8120613 | 0.0 | 0.0 |
| Kidney NAT Clontech 8120614 | 0.0 | 0.0 |
| Kidney Cancer Clontech 9010320 | 0.0 | 8.3 |
| Kidney NAT Clontech 9010321 | 43.5 | 0.0 |
| Normal Uterus GENPAK 061018 | 0.0 | 10.2 |
| Uterus Cancer GENPAK 064011 | 0.0 | 0.0 |
| Normal Thyroid Clontech A + 6570-1 | 0.0 | 0.0 |
| Thyroid Cancer GENPAK 064010 | 0.0 | 0.0 |
| Thyroid Cancer INVITROGEN A302152 | 0.0 | 0.0 |
| Thyroid NAT INVITROGEN A302153 | 0.0 | 0.0 |
| Normal Breast GENPAK 061019 | 0.0 | 0.0 |
| 84877 Breast Cancer (OD04566) | 0.0 | 0.0 |
| 85975 Breast Cancer (OD04590-01) | 0.0 | 31.2 |
| 85976 Breast Cancer Mets (OD04590-03) | 0.0 | 0.0 |
| 87070 Breast Cancer Metastasis | 0.0 | 55.3 |
| (OD04655-05) | | |
| GENPAK Breast Cancer 064006 | 15.3 | 0.0 |
| Breast Cancer Res. Gen. 1024 | 0.0 | 0.0 |
| Breast Cancer Clontech 9100266 | 0.0 | 0.0 |
| Breast NAT Clontech 9100265 | 0.0 | 0.0 |
| Breast Cancer INVITROGEN A209073 | 0.0 | 0.0 |
| Breast NAT INVITROGEN A2090734 | 0.0 | 0.0 |
| Normal Liver GENPAK 061009 | 0.0 | 0.0 |
| Liver Cancer GENPAK 064003 | 0.0 | 20.1 |
| Liver Cancer Research Genetics | 0.0 | 0.0 |
| RNA 1025 | | |
| Liver Cancer Research Genetics | 0.0 | 0.0 |
| RNA 1026 | | |
| Paired Liver Cancer Tissue Research | 0.0 | 0.0 |
| Genetics RNA 6004-T | | |
| Paired Liver Tissue Research | 0.0 | 13.9 |
| Genetics RNA 6004-N | | |
| Paired Liver Cancer Tissue Research | 0.0 | 0.0 |
| Genetics RNA 6005-T | | |
| Paired Liver Tissue Research | 0.0 | 0.0 |
| Genetics RNA 6005-N | | |
| Normal Bladder GENPAK 061001 | 0.0 | 0.0 |
| Bladder Cancer Research Genetics | 52.8 | 17.6 |
| RNA 1023 | | |
| Bladder Cancer INVITROGEN A302173 | 50.3 | 0.0 |
| 87071 Bladder Cancer (OD04718-01) | 17.0 | 0.0 |
| 87072 Bladder Normal Adjacent | 0.0 | 0.0 |
| (OD04718-03) | | |
| Normal Ovary Res. Gen. | 42.9 | 24.6 |
| Ovarian Cancer GENPAK 064008 | 0.0 | 0.0 |
| 87492 Ovary Cancer (OD04768-07) | 0.0 | 0.0 |
| 87493 Ovary NAT (OD04768-08) | 0.0 | 0.0 |
| Normal Stomach GENPAK 061017 | 0.0 | 0.0 |
| Gastric Cancer Clontech 9060358 | 0.0 | 0.0 |
| NAT Stomach Clontech 9060359 | 0.0 | 0.0 |
| Gastric Cancer Clontech 9060395 | 0.0 | 0.0 |
| NAT Stomach Clontech 9060394 | 0.0 | 0.0 |
| Gastric Cancer Clontech 9060397 | 0.0 | 0.0 |
| NAT Stomach Clontech 9060396 | 0.0 | 0.0 |
| Gastric Cancer GENPAK 064005 | 0.0 | 0.0 |

Panel 51
Panel 3D

| | Relative | | Relative |
|---|---|---|---|
| | Expression (%) | | Expression (%) |
| | 3dtm4953f_{—} | | 3dtm4953f_{—} |
| Tissue Name | ag1481 | Tissue Name | ag1481 |
| 94905_Daoy_Medulloblastoma/ | 0.0 | 94954_Ca Ski_Cervical | 0.0 |
| Cerebellum_sscDNA | | epidermoid carcinoma | |
| | | (metastasis)_sscDNA | |
| 94906_TE671_Medulloblastom/ | 0.0 | 94955_ES-2_Ovarian clear cell | 0.0 |
| Cerebellum_sscDNA | | carcinoma_sscDNA | |
| 94907_D283 | 0.0 | 94957_Ramos/6h stim_Stimulated | 0.0 |
| Med_Medulloblastoma/ | | with PMA/ionomycin 6h_sscDNA | |
| Cerebellum_sscDNA | | | |
| 94908_PFSK-1_Primitive | 0.0 | 94958_Ramos/14h stim_{—} | 0.0 |
| Neuroectodermal/Cerebellum_{—} | | Stimulated with PMA/ionomycin | |
| sscDNA | | 14h_sscDNA | |
| 94909_XF-498_CNS_sscDNA | 0.0 | 94962_MEG-01_Chronic | 0.0 |
| | | myelogenous leukemia | |
| | | (megokaryoblast)_sscDNA | |
| 94910_SNB78_{—} | 0.0 | 94963_Raji_Burkitt's | 0.0 |
| CNS/glioma_sscDNA | | lymphoma_sscDNA | |
| 94911_SF-268_{—} | 0.0 | 94964_Daudi_Burkitt's | 0.0 |
| CNS/glioblastoma_sscDNA | | lymphoma_sscDNA | |
| 94912_T98G_Glioblastoma_{—} | 0.0 | 94965_U266_B-cell | 0.0 |
| sscDNA | | plasmacytoma/myeloma_sscDNA | |
| 96776_SK-N-SH_{—} | 0.0 | 94968_CA46_Burkitt's | 0.0 |
| Neuroblastoma | | lymphoma_sscDNA | |
| (metastasis)_sscDNA | | | |
| 94913_SF295_{—} | 0.0 | 94970_RL_non-Hodgkin's B-cell | 0.0 |
| CNS/glioblastoma_sscDNA | | lymphoma_sscDNA | |
| 94914_Cerebellum_sscDNA | 0.0 | 94972_JM1_pre-B-cell | 0.0 |
| | | lymphoma/leukemia_sscDNA | |
| 96777_Cerebellum_sscDNA | 0.0 | 94973_Jurkat_T cell | 0.0 |
| | | leukemia_sscDNA | |
| 94916_NCIH292 | 0.0 | 94974_TF1_Erythroleukemia_{—} | 0.0 |
| Mucoepidermoid lung | | sscDNA | |
| carcinoma_sscDNA | | | |
| 94917_DMS-114_Small cell | 0.0 | 94975_HUT 78_T-cell | 0.0 |
| lung cancer_sscDNA | | lymphoma_sscDNA | |
| 94918_DMS-79_Small cell lung | 0.0 | 94977_U937_Histiocytic | 0.0 |
| cancer/neuroendocrine_sscDNA | | lymphoma_sscDNA | |
| 94919_NCI-H146_Small cell | 0.0 | 94980_KU-812_Myelogenous | 0.0 |
| lung | | leukemia_sscDNA | |
| cancer/neuroendocrine_sscDNA | | | |
| 94920_NCI-H526_Small cell | 0.0 | 94981_769-P_Clear cell renal | 0.0 |
| lung | | carcinoma_sscDNA | |
| cancer/neuroendocrine_sscDNA | | | |
| 94921_NCI-N417_Small cell | 0.0 | 94983_Caki-2_Clear cell renal | 0.0 |
| lung | | carcinoma_sscDNA | |
| cancer/neuroendocrine_sscDNA | | | |
| 94923_NCI-H82_Small cell lung | 0.0 | 94984_SW 839_Clear cell renal | 0.0 |
| cancer/neuroendocrine_sscDNA | | carcinoma_sscDNA | |
| 94924_NCI-H157_Squamous | 0.0 | 94986_G401_Wilms' | 0.0 |
| cell lung cancer | | tumor_sscDNA | |
| (metastasis)_sscDNA | | | |
| 94925_NCI-H1155_Large cell | 0.0 | 94987_Hs766T_Pancreatic | 0.0 |
| lung | | carcinoma (LN | |
| cancer/neuroendocrine_sscDNA | | metastasis)_sscDNA | |
| 94926_NCI-H1299_Large cell | 0.0 | 94988_CAPAN-1_Pancreatic | 0.0 |
| lung | | adenocarcinoma (liver | |
| cancer/neuroendocrine_sscDNA | | metastasis)_sscDNA | |
| 94927_NCI-H727_Lung | 0.0 | 94989_SU86.86_Pancreatic | 0.0 |
| carcinoid_sscDNA | | carcinoma (liver | |
| | | metastasis)_sscDNA | |
| 94928_NCI-UMC-11_Lung | 0.0 | 94990_BxPC-3_Pancreatic | 0.0 |
| carcinoid_sscDNA | | adenocarcinoma_sscDNA | |
| 94929_LX-1_Small cell lung | 0.0 | 94991_HPAC_Pancreatic | 0.0 |
| cancer_sscDNA | | adenocarcinoma_sscDNA | |
| 94930_Colo-205_Colon | 0.0 | 94992_MIA PaCa-2_Pancreatic | 0.0 |
| cancer_sscDNA | | carcinoma_sscDNA | |
| 94931_KM12_Colon | 0.0 | 94993_CFPAC-1_Pancreatic | 0.0 |
| cancer_sscDNA | | ductal adenocarcinoma_sscDNA | |
| 94932_KM20L2_Colon | 0.0 | 94994_PANC-1_Pancreatic | 0.0 |
| cancer_sscDNA | | eithelioid ductal | |
| | | carcinoma_sscDNA | |
| 94933_NCI-H716_Colon | 0.0 | 94996_T24_Bladder carcinma | 0.0 |
| cancer_sscDNA | | (transitional cell)_sscDNA | |
| 94935_SW-48_Colon | 0.0 | 94997_5637_Bladder | 0.0 |
| adenocarcinoma_sscDNA | | carcinoma_sscDNA | |
| 94936_SW1116_Colon | 0.0 | 94998_HT-1197_Bladder | 0.0 |
| adenocarcinoma_sscDNA | | carcinoma_sscDNA | |
| 94937_LS 174T_Colon | 0.0 | 94999_UM-UC-3_Bladder | 0.0 |
| adenocarcinoma_sscDNA | | carcinma (transitional | |
| | | cell)_sscDNA | |
| 94938_SW-948_Colon | 0.0 | 95000_A204_Rhabdomyosarcoma_{—} | 0.0 |
| adenocarcinoma_sscDNA | | sscDNA | |
| 94939_SW-480_Colon | 0.0 | 95001_HT-1080_{—} | 0.0 |
| adenocarcinoma_sscDNA | | Fibrosarcoma_sscDNA | |
| 94940_NCI-SNU-5_Gastric | 0.0 | 95002_MG-63_Osteosarcoma | 0.0 |
| carcinoma_sscDNA | | (bone)_sscDNA | |
| 94941_KATO III_Gastric | 0.0 | 95003_SK-LMS-1_{—} | 0.0 |
| carcinoma_sscDNA | | Leiomyosarcoma | |
| | | (vulva)_sscDNA | |
| 94943_NCI-SNU-16_Gastric | 0.0 | 95004_SJRH30_Rhabdomyosarcoma | 100.0 |
| carcinoma_sscDNA | | (met to bone marrow)_sscDNA | |
| 94944_NCI-SNU-1_Gastric | 0.0 | 95005_A431_Epidermoid | 0.0 |
| carcinoma_sscDNA | | carcinoma_sscDNA | |
| 94946_RF-1_Gastric | 0.0 | 95007_WM2664_{—} | 0.0 |
| adenocarcinoma_sscDNA | | Melanoma_sscDNA | |
| 94947_RF-48_Gastric | 0.0 | 95010_DU 145_Prostate | 0.0 |
| adenocarcinoma_sscDNA | | carcinoma (brain | |
| | | metastasis)_sscDNA | |
| 96778_MKN-45_Gastric | 0.0 | 95012_MDA-MB-468_Breast | 0.0 |
| carcinoma_sscDNA | | adenocarcinoma_sscDNA | |
| 94949_NCI-N87_Gastric | 0.0 | 95013_SCC-4_Squamous cell | 0.0 |
| carcinoma_sscDNA | | carcinoma of tongue_sscDNA | |
| 94951_OVCAR-5_Ovarian | 0.0 | 95014_SCC-9_Squamous cell | 0.0 |
| carcinoma_sscDNA | | carcinoma of tongue_sscDNA | |
| 94952_RL95-2_Uterine | 0.0 | 95015_SCC-15_Squamous cell | 0.0 |
| carcinoma_sscDNA | | carcinoma of tongue_sscDNA | |
| 94953_HelaS3_Cervical | 0.0 | 95017_CAL 27_Squamous cell | 0.0 |
| adenocarcinoma_sscDNA | | carcinoma of tongue_sscDNA | |

TABLE 52
Panel 4D

| | Relative | | |
|---|---|---|---|
| | Expression (%) | Relative Expression (%) | |
| | 4dx4tm4512t_{—} | 4dtm2475f_{—} | 4dtm4172f_{—} |
| Tissue Name | ag2307_a2 | ag1481 | ag1481 |
| 93768_Secondary Th1_anti-CD28/anti- | 10.4 | 0.0 | 10.7 |
| CD3 | | | |
| 93769_Secondary Th2_anti-CD28/anti- | 0.0 | 0.0 | 0.0 |
| CD3 | | | |
| 93770_Secondary Tr1_anti-CD28/anti- | 0.0 | 0.0 | 0.0 |
| CD3 | | | |
| 93573_Secondary Th1_resting day 4-6 | 2.6 | 0.0 | 12.9 |
| in IL-2 | | | |
| 93572_Secondary Th2_resting day 4-6 | 0.0 | 0.0 | 0.0 |
| in IL-2 | | | |
| 93571_Secondary Tr1_resting day 4-6 | 0.0 | 0.0 | 0.0 |
| in IL-2 | | | |
| 93568_primary Th1_anti-CD28/anti- | 2.8 | 0.0 | 0.0 |
| CD3 | | | |
| 93569_pnimary Th2_anti-CD28/anti- | 2.9 | 0.0 | 0.0 |
| CD3 | | | |
| 93570_primary Tr1_anti-CD28/anti- | 3.7 | 4.5 | 0.0 |
| CD3 | | | |
| 93565_primary Th1_resting dy 4-6 in | 0.0 | 2.9 | 0.0 |
| IL-2 | | | |
| 93566_primary Th2_resting dy 4-6 in | 0.0 | 0.0 | 0.0 |
| IL-2 | | | |
| 93567_primary Tr1_resting dy 4-6 in | 2.2 | 0.0 | 0.0 |
| IL-2 | | | |
| 93351_CD45RA CD4 | 14.7 | 21.0 | 10.3 |
| lymphocyte_anti-CD28/anti-CD3 | | | |
| 93352_CD45RO CD4 | 0.0 | 7.6 | 0.0 |
| lymphocyte_anti-CD28/anti-CD3 | | | |
| 93251_CD8 Lymphocytes_anti- | 0.0 | 9.7 | 0.0 |
| CD28/anti-CD3 | | | |
| 93353 chronic CD8 Lymphocytes | 0.0 | 0.0 | 0.0 |
| 2ry_resting dy 4-6 in IL-2 | | | |
| 93574_chronic CD8 Lymphocytes | 0.0 | 0.0 | 0.0 |
| 2ry_activated CD3/CD28 | | | |
| 93354_CD4_none | 14.8 | 0.0 | 0.0 |
| 93252_Secondary Th1/Th2/Tr1_anti- | 0.0 | 0.0 | 0.0 |
| CD95 CH11 | | | |
| 93103_LAK cells_resting | 0.6 | 0.0 | 0.0 |
| 93788_LAK cells_IL-2 | 0.0 | 0.0 | 0.0 |
| 93787_LAK cells_IL-2 + IL-12 | 23.4 | 33.0 | 0.0 |
| 93789_LAK cells_IL-2 + IFN gamma | 2.4 | 39.0 | 28.1 |
| 93790_LAK cells_IL-2 + IL-18 | 43.6 | 30.1 | 18.9 |
| 93104_LAK cells_PMA/ionomycin and | 0.7 | 0.0 | 0.0 |
| IL-18 | | | |
| 93578_NK Cells IL-2_resting | 0.0 | 0.0 | 0.0 |
| 93109_Mixed Lymphocyte | 9.5 | 19.5 | 28.3 |
| Reaction_Two Way MLR | | | |
| 93110_Mixed Lymphocyte | 0.0 | 20.6 | 0.0 |
| Reaction_Two Way MLR | | | |
| 93111_Mixed Lymphocyte | 6.0 | 0.0 | 0.0 |
| Reaction_Two Way MLR | | | |
| 93112_Mononuclear Cells | 1.0 | 7.0 | 0.0 |
| (PBMCs)_resting | | | |
| 93113_Mononuclear Cells | 8.6 | 0.0 | 0.0 |
| (PBMCs)_PWM | | | |
| 93114_Mononuclear Cells | 0.0 | 0.0 | 0.0 |
| (PBMCs)_PHA-L | | | |
| 93249_Ramos (B cell)_none | 0.0 | 0.0 | 0.0 |
| 93250_Ramos (B cell)_ionomycin | 0.0 | 0.0 | 0.0 |
| 93349_B lymphocytes_PWM | 41.6 | 70.7 | 44.8 |
| 93350_B lymphoytes_CD40L and IL-4 | 7.6 | 23.3 | 9.7 |
| 92665_EOL-1 (Eosinophil)_dbcAMP | 0.0 | 0.0 | 0.0 |
| differentiated | | | |
| 93248_EOL-1(Eosinophil)_{—} | 0.0 | 19.3 | 0.0 |
| dbcAMP/PMAionomycin | | | |
| 93356_Dendritic Cells_none | 0.0 | 0.0 | 0.0 |
| 93355_Dendritic Cells_LPS 100 ng/ml | 0.0 | 0.0 | 0.0 |
| 93775_Dendritic Cells_anti-CD40 | 0.0 | 0.0 | 0.0 |
| 93774_Monocytes_resting | 0.0 | 0.0 | 0.0 |
| 93776_Monocytes_LPS 50 ng/ml | 0.0 | 0.0 | 0.0 |
| 93581_Macrophages_resting | 0.0 | 23.5 | 0.0 |
| 93582_Macrophages_LPS 100 ng/ml | 0.0 | 0.0 | 0.0 |
| 93098_HUVEC (Endothelial)_none | 0.0 | 0.0 | 0.0 |
| 93099_HUVEC (Endothelial)_starved | 5.4 | 0.0 | 0.0 |
| 93100_HUVEC (Endothelial)_IL-1b | 0.0 | 8.4 | 0.0 |
| 93779_HUVEC (Endothelial)_{—} | 0.0 | 6.1 | 0.0 |
| IFN gamma | | | |
| 93102_HUVEC (Endothelial)_TNF | 0.0 | 0.0 | 0.0 |
| alpha + IFN gamma | | | |
| 93101_HUVEC (Endothelial)_TNF | 0.0 | 0.0 | 0.0 |
| alpha + IL4 | | | |
| 93781_HUVEC (Endothelial)_IL-11 | 0.0 | 0.0 | 0.0 |
| 93583_Lung Microvascular Endothelial | 14.8 | 0.0 | 0.0 |
| Cells_none | | | |
| 93584_Lung Microvascular Endothelial | 0.0 | 0.0 | 0.0 |
| Cells_TNFa (4 ng/ml) and IL1b | | | |
| (1 ng/ml) | | | |
| 92662_Microvascular Dermal | 0.0 | 0.0 | 0.0 |
| endothelium_none | | | |
| 92663_Microsvasular Dermal | 0.0 | 0.0 | 11.0 |
| endothelium_TNFa (4 ng/ml) and IL1b | | | |
| (1 ng/ml) | | | |
| 93773_Bronchial epithelium_TNFa | 0.0 | 0.0 | 0.0 |
| (4 ng/ml) and IL1b (1 ng/ml)** | | | |
| 93347_Small Airway Epithelium_none | 6.5 | 0.0 | 0.0 |
| 93348_Small Airway Epithelium_TNFa | 7.4 | 0.0 | 0.0 |
| (4 ng/ml) and IL1b (1 ng/ml) | | | |
| 92668_Coronery Artery SMC_resting | 0.0 | 0.0 | 0.0 |
| 92669_Coronery Artery SMC_TNFa | 0.0 | 1.2 | 0.0 |
| (4 ng/ml) and IL1b (1 ng/ml) | | | |
| 93107_astrocytes_resting | 6.9 | 0.0 | 0.0 |
| 93108_astrocytes_TNFa (4 ng/ml) and | 0.0 | 0.0 | 0.0 |
| IL1b (1 ng/ml) | | | |
| 92666_KU-812 (Basophil)_resting | 0.0 | 0.0 | 0.0 |
| 92667_KU-812 | 0.9 | 0.0 | 0.0 |
| (Basophil)_PMA/ionoycin | | | |
| 93579_CCD1106 (Keratinocytes)_none | 0.0 | 0.0 | 0.0 |
| 93580_CCD1106 | 0.0 | 0.0 | 0.0 |
| (Keratinocytes)_TNFa and IFNg** | | | |
| 93791_Liver Cirrhosis | 45.1 | 72.7 | 25.2 |
| 93792_Lupus Kidney | 9.9 | 0.0 | 0.0 |
| 93577_NCI-H292 | 0.0 | 0.0 | 0.0 |
| 93358_NCI-H292_IL-4 | 0.0 | 0.0 | 0.0 |
| 93360_NCI-H292_IL-9 | 0.0 | 0.0 | 0.0 |
| 93359_NCI-H292_IL-13 | 0.0 | 0.0 | 0.0 |
| 93357_NCI-H292_IFN gamma | 0.0 | 0.0 | 0.0 |
| 93777_HPAEC_- | 0.0 | 0.0 | 0.0 |
| 93778_HPAEC_IL-1 beta/TNA alpha | 0.0 | 0.0 | 0.0 |
| 93254_Normal Human Lung | 0.0 | 0.0 | 0.0 |
| Fibroblast_none | | | |
| 93253_Normal Human Lung | 0.0 | 0.0 | 0.0 |
| Fibroblast_TNFa (4 ng/ml) and IL-1b | | | |
| (1 ng/ml) | | | |
| 93257_Normal Human Lung | 0.0 | 0.0 | 0.0 |
| Fibroblast_IL-4 | | | |
| 93256_Normal Human Lung | 0.0 | 0.0 | 0.0 |
| Fibroblast_IL-9 | | | |
| 93255_Normal Human Lung | 7.6 | 0.0 | 0.0 |
| Fibroblast_IL-13 | | | |
| 93258_Normal Human Lung | 0.0 | 0.0 | 0.0 |
| Fibroblast_IFN gamma | | | |
| 93106_Dermal Fibroblasts | 0.0 | 0.0 | 0.0 |
| CCD1070_resting | | | |
| 93361_Dermal Fibroblasts | 0.0 | 0.0 | 0.0 |
| CCD1070_TNF alpha 4 ng/ml | | | |
| 93105_Dermal Fibroblasts | 3.2 | 0.0 | 0.0 |
| CCD1070_IL-1 beta 1 ng/ml | | | |
| 93772_dermal fibroblast_IFN gamma | 3.1 | 0.0 | 0.0 |
| 93771_dermal fibroblast_IL-4 | 0.0 | 0.0 | 0.0 |
| 93260_IBD Colitis 2 | 0.0 | 1.5 | 0.0 |
| 93261_IBD Crohns | 0.0 | 0.0 | 0.0 |
| 735010_Colon_normal | 100.0 | 100.0 | 100.0 |
| 735019_Lung_none | 60.8 | 33.2 | 47.6 |
| 64028-1_Thymus_none | 10.6 | 0.0 | 8.0 |
| 64030-1_Kidney_none | 0.0 | 1.0 | 11.0 |

TABLE 53
Panel CNS_1

| | Relative | | Relative |
|---|---|---|---|
| | Expression (%) | | Expression (%) |
| | cns1x4tm6179f_{—} | | cns1x4tm6179f_{—} |
| Tissue Name | ag1481_a1 | Tissue Name | ag1481_a1 |
| 102633_BA4 Control | 1.1 | 102605_BA17 PSP | 1.2 |
| 102641_BA4 Control2 | 4.2 | 102612_BA17 PSP2 | 0.4 |
| 102625_BA4 Alzheimer's2 | 0.4 | 102637_Sub Nigra Control | 0.7 |
| 102649_BA4 Parkinson's | 2.7 | 102645_Sub Nigra Control2 | 2.6 |
| 102656_BA4 Parkinson's2 | 4.3 | 102629_Sub Nigra Alzheimer's2 | 0.7 |
| 102664_BA4 Huntington's | 2.3 | 102660_Sub Nigra Parkinson's2 | 3.8 |
| 102671_BA4 Huntington's2 | 0.3 | 102667_Sub Nigra Huntington's | 3.0 |
| 102603_BA4 PSP | 0.3 | 102674_Sub Nigra Huntington's2 | 1.2 |
| 102610_BA4 PSP2 | 1.1 | 102614_Sub Nigra PSP2 | 0.6 |
| 102588_BA4 Depression | 0.2 | 102592_Sub Nigra Depression | 0.0 |
| 102596_BA4 Depression2 | 0.3 | 102599_Sub Nigra Depression2 | 0.3 |
| 102634_BA7 Control | 1.4 | 102636_Glob Palladus Control | 1.8 |
| 102642_BA7 Control2 | 2.1 | 102644_Glob Palladus Control2 | 2.4 |
| 102626_BA7 Alzheimer's2 | 0.3 | 102620_Glob Palladus Alzheimer's | 1.0 |
| 102650_BA7 Parkinson's | 0.4 | 102628_Glob Palladus | 0.7 |
| | | Alzheimer's2 | |
| 102657_BA7 Parkinson's2 | 1.5 | 102652_Glob Palladus Parkinson's | 9.2 |
| 102665_BA7 Huntington's | 2.6 | 102659_Glob Palladus | 2.0 |
| | | Parkinson's2 | |
| 102672_BA7 Huntington's2 | 0.6 | 102606_Glob Palladus PSP | 1.5 |
| 102604_BA7 PSP | 1.8 | 102613_Glob Palladus PSP2 | 1.5 |
| 102611_BA7 PSP2 | 1.0 | 102591_Glob Palladus Depression | 0.2 |
| 102589_BA7 Depression | 0.2 | 102638_Temp Pole Control | 1.4 |
| 102632_BA9 Control | 1.2 | 102646_Temp Pole Control2 | 2.8 |
| 102640_BA9 Control2 | 6.1 | 102622_Temp Pole Alzheimer's | 100.0 |
| 102617_BA9 Alzheimer's | 0.6 | 102630_Temp Pole Alzheimer's2 | 0.0 |
| 102624_BA9 Alzheimer's2 | 0.5 | 102653_Temp Pole Parkinson's | 0.9 |
| 102648_BA9 Parkinson's | 1.4 | 102661_Temp Pole Parkinson's2 | 0.9 |
| 102655_BA9 Parkinson's2 | 2.6 | 102668_Temp Pole Huntington's | 1.1 |
| 102663_BA9 Huntington's | 2.7 | 102607_Temp Pole PSP | 0.2 |
| 102670_BA9 Huntington's2 | 0.5 | 102615_Temp Pole PSP2 | 0.3 |
| 102602_BA9 PSP | 0.8 | 102600_Temp Pole Depression2 | 0.1 |
| 102609_BA9 PSP2 | 0.3 | 102639_Cing Gyr Control | 2.4 |
| 102587_BA9 Depression | 0.2 | 102647_Cing Gyr Control2 | 2.8 |
| 102595_BA9 Depression2 | 0.1 | 102623_Cing Gyr Alzheimer's | 0.6 |
| 102635_BA17 Control | 1.3 | 102631_Cing Gyr Alzheimer's2 | 0.2 |
| 102643_BA17 Control2 | 3.8 | 102654_Cing Gyr Parkinson's | 0.7 |
| 102627_BA17 Alzheimer's2 | 0.3 | 102662_Cing Gyr Parkinson's2 | 1.6 |
| 102651_BA17 Parkinson's | 0.7 | 102669_Cing Gyr Huntington's | 2.6 |
| 102658_BA17 Parkinson's2 | 1.7 | 102676_Cing Gyr Huntington's2 | 0.5 |
| 102666_BA17 Huntington's | 2.4 | 102608_Cing Gyr PSP | 0.4 |
| 102673_BA17 Huntington's2 | 0.3 | 102616_Cing Gyr PSP2 | 0.3 |
| 102590_BA17 Depression | 0.2 | 102594_Cing Gyr Depression | 0.1 |
| 102597_BA17 Depression2 | 1.3 | 102601_Cing Gyr Depression2 | 0.3 |

TABLE 54
Panel CNS_Neurodegeneration_v1.0

| | Relative | | Relative |
|---|---|---|---|
| | Expression (%) | | Expression (%) |
| | tm6958f_{—} | | tm6958f_{—} |
| Tissue Name | ag1481_a1s1 | Tissue Name | ag1481_a1s1 |
| AD 1 Hippo | 3.2 | Control (Path) 3 Temporal Ctx | 2.1 |
| AD 2 Hippo | 15.9 | Control (Path) 4 Temporal Ctx | 21.1 |
| AD 3 Hippo | 1.1 | AD 1 Occipital Ctx | 6.2 |
| AD 4 Hippo | 4.1 | AD 2 Occipital Ctx (Missing) | 0.0 |
| AD 5 Hippo | 92.1 | AD 3 Occipital Ctx | 1.5 |
| AD 6 Hippo | 20.4 | AD 4 Occipital Ctx | 12.4 |
| Control 2 Hippo | 28.9 | AD 5 Occipital Ctx | 61.1 |
| Control 4 Hippo | 1.7 | AD 6 Occipital Ctx | 12.2 |
| Control (Path) 3 Hippo | 0.7 | Control 1 Occipital Ctx | 0.8 |
| AD 1 Temporal Ctx | 5.0 | Control 2 Occipital Ctx | 89.8 |
| AD 2 Temporal Ctx | 18.6 | Control 3 Occipital Ctx | 10.6 |
| AD 3 Temporal Ctx | 1.7 | Control 4 Occipital Ctx | 0.2 |
| AD 4 Temporal Ctx | 9.9 | Control (Path) 1 Occipital Ctx | 69.1 |
| AD 5 Inf Temporal Ctx | 88.6 | Control (Path) 2 Occipital Ctx | 5.1 |
| AD 5 Sup Temporal Ctx | 16.9 | Control (Path) 3 Occipital Ctx | 0.4 |
| AD 6 Inf Temporal Ctx | 17.1 | Control (Path) 4 Occipital Ctx | 12.0 |
| AD 6 Sup Temporal Ctx | 23.4 | Control 1 Parietal | 2.1 |
| Control 1 Temporal Ctx | 1.8 | Control 2 Parietal | 17.7 |
| Control 2 Temporal Ctx | 62.9 | Control 3 Parietal | 15.4 |
| Control 3 Temporal Ctx | 8.3 | Control (Path) 1 Parietal | 100.0 |
| Control 3 Temporal Ctx | 3.3 | Control (Path) 2 Parietal | 15.3 |
| Control (Path) 1 Temporal Ctx | 51.6 | Control (Path) 3 Parietal | 1.2 |
| Control (Path) 2 Temporal Ctx | 25.7 | Control (Path) 4 Parietal | 32.8 |

Panel 1.3D Summary Ag1481/Ag2307 Results from experiments using different probe and prime sets are in very good agreement. The NOV7a gene is expressed at high to moderate levels in all the tissue samples originating from the central nervous system, including the pituitary gland, amygdala, cerebellum, hippocampus, substantia nigra, thalamus, cerebral cortex and spinal cord. Highest expression is detected in the hippocampus region (CT=27-30). These high expresson levels of the NOV7a gene suggest that the NOV7a protein product may be essential for normal central nervous system function. Thus, expression of the NOV7a gene could potentially be used to distinguish brain tissues from other tissues and may also be an excellent target in the treatment of neurpsychiatric disease.

Moderate expression of the NOV7a gene also occurs in lung cancer cell lines (CT=30.4, 31.3). Therefore, in addition to its CNS utility, expression of the NOV7a gene could potentially be used to distinguish between lung cancer cell lines and other tissues or cell lines.

Panel General_screening_panel_v1.4 Summary Ag1481 Results from two experiments using the same probe and primer set are in excellent agreement. Highest expression of the NOV7a gene is seen in the cerebellum (CT=25) with significant expression detected in all the tissues samples originating from regions of the brain including the amygdala, hippocampus, cerebral cortex, substantia nigra, thalamus, and spinal cord. In addition, high expression of the NOV7a gene is present in lung cancer cell lines (CT=26) The results from this panel are in excellent agreement with the expression profile detected in panel 1.3D. Therefore, these results suggest that expression of the NOV7a gene could be used to distinguish normal brain tissue from other tissues. The NOV7a gene could also possibly serve as a marker of lung cancer cell lines from other cell lines and tissues.

The metabolic expression of the NOV7a gene is limited to the pituitary gland with CT values ranging from 29-32. Therefore, the NOV7a protein product may be a small molecule target for the treatment of diseases involving the pituitary gland.

Panel 2D Summary Ag1481 Among the tissues samples in this panel, expression of the NOV7a gene is low but significant in normal colon tissue adjacent to a colon tumor (CT=34) as well as in a lung cancer metastasis to muscle (CT=34.8). Two replicates of this experiment follow similar trends with both showing no expression in most tissue samples. Thus, this expression profile suggests that expression of the NOV7a gene could potentially be used to distinguish between colon cancer and normal tissue.

Panel 2.2 Summary Ag2307 Expression of this gene in panel 2.2 is low/undetectable (Ct values>35) in all samples (data not shown).

Panel 3D Summary Ag1481 High expression of the NOV7a gene is detected in a metastatic rhabdomyosarcoma cell line with a CT value of 18.2. Moderate expression is also detected in two lung cancer samples (small cell CT=33.1; large cell CT=32.8) and cerebellum (CT=28.2). Thus, this gene could be used to distinguish these samples from other cell line samples.

Panel 4D Summary Ag1481/Ag2307 Results from two experiments using two different probe/primer sets are in excellent agreement. Expression of the NOV7a gene is greatest in tissue derived from normal colon (CT=32) and is also observed at moderate levels in lung tissue (CT=33), mitogen stimulated B cells (CT=32-33), LAK cells stimulated with IL-2 and gamma interferon (CT=33-34), and LAK cells treated with IL-2 and IL-18 (CT=34). The NOV7a gene encodes a protein with homology to vesicular GABA tranporters that may be active in regulating secretion within the colon and perhaps the lung. The function of this type of transporter in leukocytes has not been described. Therepeutic regulation of the protein encoded by NOV7a could be important in the treatment of colitis as well as diseases involving the lung, including asthma and emphysema.

Panel CNS_{—}1 Summary Ag1481 The NOV7a gene is widely expressed at low to moderate levels in most of the tissue samples in this panel. Expression of the gene is highest (CT=25) in the temporal pole from an Alzheimer's patient. Panel CNS.01 also shows the NOV7a gene to be downregulated in the parietal, prefrontal, and cingulated cortex of depressed patients. It could therefore make an excellent drug target for schizophrenia. Multiple laboratories have shown a GABAergic deficit in schizophrenia and bipolar diaorder, usually a decrease in the number of intemeurons producing GABA. Thus, therapeutic modulation or potentiation of this protein to increase the amount of GABA transported to the synaptic vesicles could be of benefit in schizoprenia and/or bipolar disorder. Furthermore, the gene for this protein is located on chromosome 20 (specifically at 20q12), a locus that has been linked to schizophrenia. This information, when coupled with the fact that at least 4 amino acid changing SNPs exist in the coding region of this gene, make the NOV7a gene an excellent candidate for screening for risk of psychiatric disease.

Panel CNS_Neurodegeneration_v1.0 Summary Ag1481 The NOV7a gene shows expression at moderate to low levels in most of the tissues in this sample. Highest expression is detected in the parietal cortex of a control patient (CT=28.5). Other tissue samples showing moderate levels of expression of the NOV7a gene include the occipital cortex (CT=29), and temporal cortex (CT=29.5) region of a control patient and the occipital cortex (CT=29.2), inferior temporal cortex (CT=29.7) and hippocampus regions of an Alzheimer's patient (CT=28.6). Based on this expression profile, this gene does not appear to be differentially regulated in Alzheimer's disease, although this panel does confirm that this gene is expressed at moderate to high levels in the CNS.

This protein appears to be the human homologue of the rat vesicular GABA transporter (VGAT). GABA, the primary inhibitory neurotransmitter in the mammalian brain, is synthesized from glutamate in the cytoplasm by two isoforms of glutamic acid decarboxylase (GAD65 and GAD67). As with the monoamine neurotransmitters, a vesicular transporter is then necessary to transport the transmitter into the synaptic vesicle. This protein is thus critical for normal CNS function and would make an excellent drug target in neuropsychiatric disease. A large number of antiepileptics have been shown to work by either potentiating GABA transmission, or by increasing GABA production in interneurons. Therefore, therapeutic induction of the NOV7a gene or its activity may be of benefit in the control of seizures. (Gurling et al., Am J Hum Genet 68:661-73, 2001; Reynolds and Beasley, J Chem Neuroanat 22:95-100, 2001; Moshe, Neurology 55:S32-40; discussion S54-8, 2000; Timmermans and Scheuermann, Eur J Morphol 36:133-42, 1998.)

NOV10: UNC5 Receptor-like

Expression of the NOV10 gene (SC121209524_A) was assessed using the primer-probe sets Ag1522, Ag1848, Ag2263, and Ag2422 described in Tables 55, 56, 57, and 58. Results of the RTQ-PCR runs are shown in Tables 59, 60, 61, 62, 63, 64, and 65.

TABLE 55

Probe Name Ag1522

| | | | | | |
|---|---|---|---|---|---|
| | | | | Start | |
| Primers | Sequences | TM | Length | Position | SEQ ID NO: |

| | | | | | |
|---|---|---|---|---|---|
| Forward | 5′-TGACTTCGACACAGACATCACT-3′ | 58.4 | 22 | 1275 | 176 |
| Probe | TET-5′-ACTCATCTGCTGCCCTGACTGGTG- | 69.2 | 24 | 1298 | 177 |
| | 3′-TAMRA | | | | |
| Reverse | 5′-CCTTGCCGTCTTAAAGTTGAC-3′ | 58.9 | 21 | 1333 | 178 |

TABLE 56

Probe Name Ag1848

| | | | | | |
|---|---|---|---|---|---|
| | | | | Start | |
| Primers | Sequences | TM | Length | Position | SEQ ID NO: |

| | | | | | |
|---|---|---|---|---|---|
| Forward | 5′-TGACTTCGACACAGACATCACT-3′ | 58.4 | 22 | 1242 | 179 |
| Probe | TET-5′-ACTCATCTGCTGCCCTGACTGGTG- | 69.2 | 24 | 1265 | 180 |
| | 3′-TAMRA | | | | |
| Reverse | 5′-CCTTGCCGTCTTAAAGTTGAC-3′ | 58.9 | 21 | 1300 | 181 |

TABLE 57

Probe Name Ag2263

| | | | | | |
|---|---|---|---|---|---|
| | | | | Start | |
| Primers | Sequences | TM | Length | Position | SEQ ID NO: |

| | | | | | |
|---|---|---|---|---|---|
| Forward | 5′-TGACTTCGACACAGACATCACT-3′ | 58.4 | 22 | 1234 | 182 |
| Probe | TET-5′-ACTCATCTGCTGCCCTGACTGGTG- | 69.2 | 24 | 1257 183 | |
| | 3′-TAMRA | | | | |
| Reverse | 5′-CCTTGCCGTCTTAAAGTTGAC-3′ | 58.9 | 21 | 1292 | 184 |

TABLE 58

Probe Name Ag2422

| | | | | | |
|---|---|---|---|---|---|
| | | | | Start | |
| Primers | Sequences | TM | Length | Position | SEQ ID NO: |

| | | | | | |
|---|---|---|---|---|---|
| Forward | 5′-GGCTCCCTGGACACTCTCT-3′ | 59.4 | 19 | 2617 | 185 |
| Probe | FAM-5′- | 71 | 26 | 2654 | 186 |
| | CTGTCACCACCCAGCTGGGACCTTAT-3′- | | | | |
| | TAMRA | | | | |
| Reverse | 5′-TGGACAGTGGGATCTTGAAG-3′ | 58.6 | 20 | 2682 | 187 |

TABLE 59
Panel 1.2

| | Relative | | Relative |
|---|---|---|---|
| | Expression (%) | | Expression (%) |
| | 1.2tm2156t_{—} | | 1.2tm2156t_{—} |
| Tissue Name | ag1522 | Tissue Name | ag1522 |
| Endothelial cells | 1.2 | Renal ca. 786-0 | 0.0 |
| Heart (fetal) | 17.9 | Renal ca. A498 | 0.3 |
| Pancreas | 0.7 | Renal ca. RXF 393 | 0.2 |
| Pancreatic ca. CAPAN 2 | 4.9 | Renal ca. ACHN | 0.0 |
| Adrenal Gland (new lot*) | 7.9 | Renal ca. UO-31 | 0.5 |
| Thyroid | 0.0 | Renal ca. TK-10 | 0.3 |
| Salavary gland | 2.5 | Liver | 2.4 |
| Pituitary gland | 0.1 | Liver (fetal) | 0.5 |
| Brain (fetal) | 0.2 | Liver ca. (hepatoblast) HepG2 | 0.3 |
| Brain (whole) | 3.2 | Lung | 0.3 |
| Brain (amygdala) | 4.4 | Lung (fetal) | 0.4 |
| Brain (cerebellum) | 9.0 | Lung ca. (small cell) LX-1 | 25.3 |
| Brain (hippocampus) | 18.9 | Lung ca. (small cell) NCI-H69 | 43.8 |
| Brain (thalamus) | 15.7 | Lung ca. (s.cell var.) SHP-77 | 0.3 |
| Cerebral Cortex | 35.4 | Lung ca. (large cell) NCI-H460 | 54.7 |
| Spinal cord | 1.6 | Lung ca. (non-sm.cell) A549 | 0.3 |
| CNS ca. (glio/astro) U87-MG | 72.2 | Lung ca. (non-s.cell) NCI-H23 | 2.4 |
| CNS ca. (glio/astro) U-118-MG | 3.1 | Lung ca. (non-s.cell) HOP-62 | 1.7 |
| CNS ca. (astro) SW1783 | 0.3 | Lung ca. (non-s.cl) NCI-H522 | 9.3 |
| CNS ca.* (neuro; met) SK-N-AS | 36.3 | Lung ca. (squam.) SW 900 | 1.5 |
| CNS ca. (astro) SF-539 | 5.8 | Lung ca. (squam.) NCI-H596 | 22.4 |
| CNS ca. (astro) SNB-75 | 1.7 | Mammary gland | 1.4 |
| CNS ca. (glio) SNB-19 | 23.8 | Breast ca.* (pl. effusion) MCF-7 | 0.8 |
| CNS ca. (glio) U251 | 2.9 | Breast ca.* (pl.ef) MDA-MB-231 | 0.0 |
| CNS ca. (glio) SF-295 | 100.0 | Breast ca.* (pl. effusion) T47D | 18.4 |
| Heart | 31.6 | Breast ca. BT-549 | 0.0 |
| Skeletal Muscle (new lot*) | 3.4 | Breast ca. MDA-N | 0.0 |
| Bone marrow | 0.2 | Ovary | 6.9 |
| Thymus | 0.2 | Ovarian ca. OVCAR-3 | 1.7 |
| Spleen | 2.1 | Ovarian ca. OVCAR-4 | 12.9 |
| Lymph node | 0.5 | Ovarian ca. OVCAR-5 | 5.7 |
| Colorectal | 1.4 | Ovarian ca. OVCAR-8 | 5.3 |
| Stomach | 1.3 | Ovarian ca. IGROV-1 | 0.8 |
| Small intestine | 3.3 | Ovarian ca.* (ascites) SK-OV-3 | 5.4 |
| Colon ca. SW480 | 0.8 | Uterus | 0.9 |
| Colon ca.* (SW480 met) SW620 | 2.2 | Placenta | 0.9 |
| Colon ca. HT29 | 0.1 | Prostate | 10.0 |
| Colon ca. HCT-116 | 7.5 | Prostate ca.* (bone met) PC-3 | 0.0 |
| Colon ca. CaCo-2 | 6.3 | Testis | 0.3 |
| 83219 CC Well to Mod Diff | 3.0 | Melanoma Hs688(A).T | 21.2 |
| (ODO3866) | | | |
| Colon ca. HCC-2998 | 1.2 | Melanoma* (met) Hs688(B).T | 28.5 |
| Gastric ca.* (liver met) NCI-N87 | 24.7 | Melanoma UACC-62 | 2.4 |
| Bladder | 12.8 | Melanoma M14 | 0.0 |
| Trachea | 0.3 | Melanoma LOX IMVI | 0.0 |
| Kidney | 19.2 | Melanoma* (met) SK-MEL-5 | 1.2 |
| Kidney (fetal) | 6.6 | | |

TABLE 60
Panel 1.3D

| | Relative | Relative | Relative | Relative |
|---|---|---|---|---|
| | Expression(%) | Expression(%) | Expression(%) | Expression(%) |
| Tissue Name | 1.3dtm4258t_ag1522 | 1.3dtm4351t_ag1848 | 1.3dx4tm5633t_ag2263_b1 | 1.3dtm4220_ag2422 |
| Liver adenocarcinoma | 15.8 | 12.3 | 31.5 | 18.3 |
| Pancreas | 1.7 | 1.4 | 2.8 | 2.9 |
| Pancreatic ca. CAPAN 2 | 6.7 | 4.6 | 21.7 | 5.5 |
| Adrenal gland | 3.9 | 2.0 | 3.5 | 3.0 |
| Thyroid | 1.7 | 1.5 | 0.0 | 2.5 |
| Salivary gland | 0.6 | 0.2 | 2.3 | 0.3 |
| Pituitary gland | 2.1 | 1.4 | 2.9 | 4.3 |
| Brain (fetal) | 1.4 | 1.1 | 3.5 | 1.1 |
| Brain (whole) | 28.7 | 13.5 | 43.1 | 10.4 |
| Brain (amygdala) | 16.8 | 13.0 | 31.3 | 18.6 |
| Brain (cerebellum) | 8.2 | 6.5 | 42.3 | 9.2 |
| Brain (hippocampus) | 60.7 | 47.6 | 16.8 | 51.8 |
| Brain (substantia nigra) | 8.9 | 5.2 | 32.3 | 6.8 |
| Brain (thalamus) | 40.1 | 22.2 | 62.0 | 19.8 |
| Cerebral Cortex | 25.9 | 18.4 | 36.6 | 14.3 |
| Spinal cord | 10.2 | 5.4 | 38.0 | 7.9 |
| CNS ca. (glio/astro) U87- | 43.2 | 34.6 | 100.0 | 48.6 |
| MG | | | | |
| CNS ca. (glio/astro) U-118- | 10.2 | 8.0 | 6.4 | 7.5 |
| MG | | | | |
| CNS ca. (astro) SW1783 | 0.9 | 0.8 | 2.8 | 1.1 |
| CNS ca.* (neuro; met) SK- | 100.0 | 100.0 | 59.1 | 100.0 |
| N-AS | | | | |
| CNS Ca. (astro) SF-539 | 9.7 | 8.3 | 17.6 | 9.0 |
| CNS Ca. (astro) SNB-75 | 12.9 | 12.1 | 8.4 | 12.1 |
| CNS ca. (glio) SNB-19 | 19.5 | 17.6 | 46.2 | 17.2 |
| CNS Ca. (glio) U251 | 13.4 | 10.6 | 24.5 | 10.9 |
| CNS Ca. (glio) SF-295 | 66.9 | 62.4 | 64.1 | 62.0 |
| Heart (fetal) | 15.6 | 12.5 | 20.0 | 18.7 |
| Heart | 2.2 | 1.1 | 3.4 | 3.3 |
| Fetal Skeletal | 22.2 | 14.0 | 6.7 | 19.3 |
| Skeletal muscle | 0.3 | 0.2 | 1.4 | 0.7 |
| Bone marrow | 0.7 | 0.3 | 0.4 | 0.8 |
| Thymus | 2.0 | 1.6 | 3.6 | 3.4 |
| Spleen | 7.9 | 5.6 | 4.5 | 5.9 |
| Lymph node | 2.6 | 1.9 | 2.7 | 2.1 |
| Colorectal | 4.7 | 9.2 | 12.8 | 10.3 |
| Stomach | 6.1 | 2.4 | 3.6 | 4.5 |
| Small intestine | 2.9 | 2.9 | 4.5 | 4.9 |
| Colon Ca. SW480 | 2.0 | 1.0 | 1.9 | 1.5 |
| Colon ca.* (SW480 | 1.0 | 1.2 | 2.0 | 2.1 |
| met)SW620 | | | | |
| Colon ca. HT29 | 0.0 | 0.1 | 0.0 | 0.0 |
| Colon Ca. HCT-116 | 4.2 | 2.9 | 4.7 | 5.6 |
| Colon Ca. CaCo-2 | 5.3 | 3.9 | 12.5 | 7.2 |
| 83219 CC Well to Mod Diff | 14.8 | 17.3 | 19.8 | 23.5 |
| (0D03866) | | | | |
| Colon ca. HCC-2998 | 0.7 | 1.6 | 0.0 | 0.5 |
| Gastric ca.* (liver met) NCI- | 21.9 | 22.8 | 19.1 | 25.7 |
| 87 | | | | |
| Bladder | 2.1 | 1.7 | 3.4 | 1.5 |
| Trachea | 12.2 | 6.8 | 1.6 | 13.8 |
| Kidney | 1.4 | 0.6 | 3.9 | 3.0 |
| Kidney (fetal) | 5.3 | 5.8 | 5.2 | 6.3 |
| Renal ca. 786-0 | 0.0 | 0.0 | 0.0 | 0.0 |
| Renal ca. A498 | 7.7 | 7.9 | 6.8 | 9.7 |
| Renal ca. RXF 393 | 0.1 | 3.6 | 0.8 | 0.0 |
| Renal ca. ACHN | 0.0 | 0.0 | 0.0 | 0.0 |
| Renal ca. UO-31 | 0.2 | 0.3 | 0.5 | 0.3 |
| Renal Ca. TK-10 | 0.0 | 0.0 | 0.0 | 0.0 |
| Liver | 0.3 | 0.0 | 0.0 | 0.6 |
| Liver (fetal) | 1.1 | 1.0 | 0.4 | 1.2 |
| Liver ca. (hepatoblast) | 0.2 | 0.0 | 0.8 | 0.3 |
| HepG2 | | | | |
| Lung | 8.2 | 9.4 | 4.1 | 10.3 |
| Lung (fetal) | 4.3 | 4.2 | 7.3 | 4.5 |
| Lung ca. (small cell) LX-1 | 8.4 | 6.9 | 31.8 | 9.9 |
| Lung ca. (small cell) NCI- | 44.4 | 48.6 | 90.7 | 54.3 |
| H69 | | | | |
| Lung Ca. (s. cell var.) SHP- | 0.7 | 0.8 | 0.5 | 1.1 |
| 77 | | | | |
| Lung ca. (large cell)NCI- | 16.2 | 11.9 | 22.4 | 12.1 |
| H460 | | | | |
| Lung Ca. (non-sm. cell) | 0.4 | 0.3 | 0.2 | 0.4 |
| A549 | | | | |
| Lung Ca. (non-s. cell) NCI- | 2.0 | 0.9 | 3.3 | 1.2 |
| H23 | | | | |
| Lung ca (non-s. cell) HOP- | 0.4 | 0.9 | 1.6 | 0.7 |
| 62 | | | | |
| Lung Ca. (non-s. cl) NCI- | 1.7 | 0.8 | 1.7 | 1.1 |
| H522 | | | | |
| Lung Ca. (squam.) SW900 | 0.5 | 0.3 | 1.9 | 0.2 |
| Lung ca. (squam.) NCI- | 4.0 | 4.1 | 26.4 | 2.4 |
| H596 | | | | |
| Mammary gland | 6.3 | 4.4 | 3.0 | 2.8 |
| Breast ca.* (pl. effusion) | 1.1 | 0.4 | 1.5 | 0.9 |
| MCF-7 | | | | |
| Breast ca.* (pl. ef) MDA- | 0.8 | 1.2 | 0.7 | 1.4 |
| MB-231 | | | | |
| Breast ca.* (pl. effusion) | 9.6 | 5.7 | 14.0 | 4.4 |
| T47D | | | | |
| Breast ca. BT-549 | 0.2 | 0.3 | 0.2 | 0.3 |
| Breast ca. MDA-N | 0.0 | 0.0 | 0.0 | 0.0 |
| Ovary | 6.4 | 4.9 | 6.2 | 9.5 |
| Ovarian ca. OVCAR-3 | 1.1 | 0.6 | 1.1 | 0.8 |
| Ovarian ca. OVCAR-4 | 1.0 | 1.4 | 11.4 | 1.5 |
| Ovarian ca. OVCAR-5 | 2.4 | 2.6 | 5.7 | 3.3 |
| Ovarian ca. OVCAR-8 | 3.6 | 1.6 | 2.6 | 5.4 |
| Ovarian ca. IGROV-1 | 0.6 | 0.2 | 0.7 | 0.2 |
| Ovarian ca.* (ascites) SK- | 2.0 | 2.6 | 2.1 | 1.1 |
| OV-3 | | | | |
| Uterus | 2.7 | 1.3 | 3.9 | 4.2 |
| Placenta | 2.0 | 2.0 | 5.8 | 4.8 |
| Prostate | 4.4 | 2.5 | 3.4 | 5.4 |
| Prostate ca.* (bone met)PC-3 | 0.1 | 0.0 | 0.2 | 0.0 |
| Testis | 8.1 | 5.5 | 3.5 | 6.4 |
| Melanoma Hs688(A).T | 31.6 | 25.0 | 59.7 | 27.4 |
| Melanoma* (met) | 46.0 | 17.1 | 87.3 | 28.5 |
| Hs688(B).T | | | | |
| Melanoma UACC-62 | 0.1 | 0.2 | 2.0 | 0.5 |
| Melanoma M14 | 0.0 | 0.0 | 0.0 | 0.0 |
| Melanoma LOX IMVI | 0.1 | 0.2 | 0.0 | 0.0 |
| Melanoma* (met) SK-MEL-5 | 0.9 | 0.9 | 1.7 | 0.6 |
| Adipose | 3.6 | 2.3 | 5.1 | 2.9 |

TABLE 61
Panel 2D

| | Relative | Relative | | | Relative |
|---|---|---|---|---|---|
| | Expression (%) | Expression (%) | Relative Expression (%) | | Expression (%) |
| Tissue Name | 2dtm4352t_ag1848 | 2dtm5513t_ag2263 | 2Dtm2353t_ag1522 | 2dtm2417t_ag1522 | 2dtm4221f_ag2422 |
| Normal Colon GENPAK | 35.1 | 59.0 | 20.2 | 46.0 | 36.9 |
| 061003 | | | | | |
| 83219 CC Well to Mod Diff | 22.5 | 21.8 | 15.3 | 45.1 | 21.3 |
| (ODO3866) | | | | | |
| 83220 CC NAT (ODO3866) | 7.4 | 7.7 | 6.1 | 15.2 | 5.5 |
| 83221 CC Gr. 2 rectosigmoid | 5.8 | 5.9 | 7.0 | 8.2 | 13.2 |
| (ODO3868) | | | | | |
| 83222 CC NAT (ODO3868) | 0.5 | 9.3 | 0.3 | 0.5 | 0.8 |
| 83235 CC Mod Diff | 2.5 | 5.6 | 1.2 | 4.0 | 5.8 |
| (ODO3920) | | | | | |
| 83236 CC NAT (ODO3920) | 4.1 | 5.4 | 3.0 | 4.7 | 7.2 |
| 83237 CC Gr. 2 ascend colon | 24.1 | 19.9 | 10.7 | 22.5 | 25.5 |
| (ODO3921) | | | | | |
| 83238 CC NAT (ODO3921) | 7.3 | 5.6 | 3.6 | 4.3 | 5.8 |
| 83241 CC from Partial | 20.7 | 19.3 | 12.1 | 19.9 | 27.0 |
| Hepatectomy (ODO4309) | | | | | |
| 83242 Liver NAT | 2.4 | 2.6 | 0.4 | 3.6 | 3.3 |
| (ODO4309) | | | | | |
| 87472 Colon mets to lung | 6.1 | 8.5 | 5.8 | 11.9 | 10.7 |
| (OD04451-01) | | | | | |
| 87473 Lung NAT | 7.7 | 10.0 | 9.3 | 17.7 | 15.4 |
| (OD04451-02) | | | | | |
| Normal Prostate Clontech | 7.3 | 21.6 | 10.5 | 51.0 | 7.0 |
| A+ 6546-1 | | | | | |
| 84140 Prostate Cancer | 14.9 | 9.0 | 12.2 | 14.9 | 17.4 |
| (OD04410) | | | | | |
| 84141 Prostate NAT | 25.3 | 19.2 | 14.6 | 13.8 | 29.7 |
| (OD04410) | | | | | |
| 87073 Prostate Cancer | 22.7 | 31.6 | 12.2 | 18.0 | 30.6 |
| (OD04720-01) | | | | | |
| 87074 Prostate NAT | 17.7 | 16.7 | 11.8 | 11.8 | 25.0 |
| (OD04720-02) | | | | | |
| Normal Lung GENPAK | 17.6 | 12.8 | 7.3 | 17.8 | 22.4 |
| 061010 | | | | | |
| 83239 Lung Met to Muscle | 25.0 | 31.0 | 12.7 | 27.4 | 22.1 |
| (ODO4286) | | | | | |
| 83240 Muscle NAT | 6.2 | 7.3 | 7.4 | 8.7 | 9.5 |
| (ODO4286) | | | | | |
| 84136 Lung Malignant | 26.1 | 28.3 | 22.7 | 27.4 | 20.4 |
| Cancer (OD03126) | | | | | |
| 84137 Lung NAT | 21.9 | 13.9 | 12.7 | 21.9 | 31.9 |
| (OD03126) | | | | | |
| 84871 Lung Cancer | 41.5 | 30.4 | 17.9 | 41.5 | 48.0 |
| (OD04404) | | | | | |
| 84872 Lung NAT | 10.0 | 11.8 | 16.4 | 28.7 | 12.4 |
| (OD04404) | | | | | |
| 84875 Lung Cancer | 28.5 | 27.9 | 22.5 | 38.2 | 40.6 |
| (OD04565) | | | | | |
| 84876 Lung NAT | 8.5 | 8.6 | 8.1 | 11.7 | 16.3 |
| (OD04565) | | | | | |
| 85950 Lung Cancer | 10.9 | 8.8 | 9.8 | 7.1 | 9.6 |
| (OD04237-01) | | | | | |
| 85970 Lung NAT | 14.3 | 14.0 | 12.9 | 23.0 | 16.0 |
| (OD04237-02) | | | | | |
| 83255 Ocular Mel Met to | 0.7 | 0.5 | 0.6 | 0.5 | 1.1 |
| Liver (ODO4310) | | | | | |
| 83256 Liver NAT | 1.8 | 3.3 | 3.5 | 2.6 | 3.0 |
| (ODO4310) | | | | | |
| 84139 Melanoma Mets to | 3.6 | 4.3 | 1.4 | 2.0 | 2.9 |
| Lung (OD04321) | | | | | |
| 84138 Lung NAT | 25.2 | 24.0 | 20.4 | 14.4 | 18.6 |
| (OD04321) | | | | | |
| Normal Kidney GENPAK | 18.0 | 17.4 | 20.2 | 19.9 | 26.1 |
| 061008 | | | | | |
| 83786 Kidney Ca, Nuclear | 2.9 | 2.7 | 1.7 | 4.2 | 4.9 |
| grade 2 (OD04338) | | | | | |
| 83787 Kidney NAT | 17.2 | 11.3 | 6.2 | 11.7 | 22.8 |
| (OD04338) | | | | | |
| 83788 Kidney Ca Nuclear | 3.7 | 4.6 | 3.6 | 10.0 | 6.6 |
| grade 1/2(OD04339) | | | | | |
| 83789 Kidney NAT | 11.4 | 12.1 | 11.7 | 12.2 | 11.0 |
| (OD04339) | | | | | |
| 83790 Kidney Ca, Clear cell | 66.0 | 65.1 | 46.7 | 50.7 | 70.7 |
| type (OD04340) | | | | | |
| 83791 Kidney NAT | 14.8 | 12.9 | 15.3 | 19.1 | 16.8 |
| (OD04340) | | | | | |
| 83792 Kidney Ca, Nuclear | 16.3 | 16.8 | 21.0 | 9.5 | 17.0 |
| grade 3 (OD04348) | | | | | |
| 83793 Kidney NAT | 8.8 | 11.5 | 8.2 | 5.8 | 9.3 |
| (OD04348) | | | | | |
| 87474 Kidney Cancer | 27.7 | 24.8 | 24.0 | 25.3 | 41.5 |
| (OD04622-01) | | | | | |
| 87475 Kidney NAT | 3.4 | 3.1 | 2.1 | 4.6 | 5.9 |
| (OD04622-03) | | | | | |
| 85973 Kidney Cancer | 0.2 | 0.5 | 0.2 | 0.0 | 0.5 |
| (OD04450-01) | | | | | |
| 85974 Kidney NAT | 9.3 | 9.9 | 5.9 | 6.3 | 12.9 |
| (OD04450-03) | | | | | |
| Kidney Cancer Clontech | 11.9 | 12.8 | 7.3 | 9.1 | 13.4 |
| 8120607 | | | | | |
| Kidney NAT Clontech | 7.9 | 5.6 | 12.2 | 6.2 | 8.0 |
| 8120608 | | | | | |
| Kidney Cancer Clontech | 5.2 | 8.8 | 3.6 | 8.0 | 10.1 |
| 8120613 | | | | | |
| Kidney NAT Clontech | 8.9 | 7.5 | 6.2 | 6.7 | 9.3 |
| 8120614 | | | | | |
| Kidney Cancer Clontech | 25.0 | 21.9 | 18.7 | 61.1 | 22.1 |
| 9010320 | | | | | |
| Kidney NAT Clontech | 16.4 | 12.9 | 14.0 | 20.3 | 17.9 |
| 9010321 | | | | | |
| Normal Uterus GENPAK | 3.3 | 8.4 | 4.1 | 5.6 | 6.0 |
| 061018 | | | | | |
| Uterus Cancer GENPAK | 17.1 | 11.7 | 9.6 | 10.7 | 15.6 |
| 064011 | | | | | |
| Normal Thyroid Clontech | 2.6 | 1.5 | 2.6 | 9.2 | 3.6 |
| A+ 6570-1 | | | | | |
| Thyroid Cancer GENPAK | 100.00 | 82.9 | 100.0 | 72.7 | 100.0 |
| 064010 | | | | | |
| Thyroid Cancer | 12.5 | 8.0 | 7.6 | 4.5 | 11.7 |
| INVITROGEN A302152 | | | | | |
| Thyroid NAT | 2.8 | 3.2 | 3.0 | 2.4 | 6.0 |
| INVITROGEN A302153 | | | | | |
| Normal Breast GENPAK | 9.9 | 12.9 | 10.3 | 5.7 | 7.2 |
| 061019 | | | | | |
| 84877 Breast Cancer | 12.8 | 12.9 | 11.7 | 15.9 | 12.8 |
| (OD04566) | | | | | |
| 85975 Breast Cancer | 27.2 | 16.5 | 17.9 | 39.0 | 25.3 |
| (OD04590-01) | | | | | |
| 85976 Breast Cancer Mets | 35.4 | 42.0 | 26.1 | 66.0 | 27.9 |
| (OD04590-03) | | | | | |
| 87070 Breast Cancer | 6.0 | 5.2 | 4.5 | 5.4 | 3.5 |
| Metastasis (OD04655-05) | | | | | |
| GENPAK Breast Cancer | 28.1 | 21.6 | 30.8 | 32.1 | 36.3 |
| 064006 | | | | | |
| Breast Cancer Res. Gen. | 19.8 | 16.7 | 20.7 | 46.7 | 14.8 |
| 1024 | | | | | |
| Breast Cancer Clontech | 13.9 | 11.0 | 13.1 | 15.9 | 22.1 |
| 9100266 | | | | | |
| Breast NAT Clontech | 15.6 | 16.4 | 10.4 | 14.4 | 20.9 |
| 9100265 | | | | | |
| Breast Cancer | 34.2 | 25.5 | 22.2 | 26.8 | 50.0 |
| INVITROGEN A209073 | | | | | |
| Breast NAT INVITROGEN | 7.1 | 4.3 | 6.7 | 9.7 | 11.3 |
| A2090734 | | | | | |
| Normal Liver GENPAK | 1.6 | 1.7 | 1.4 | 4.2 | 2.3 |
| 061009 | | | | | |
| Liver Cancer GENPAK | 1.7 | 1.3 | 1.0 | 2.8 | 1.3 |
| 064003 | | | | | |
| Liver Cancer Research | 3.3 | 2.3 | 1.4 | 1.1 | 3.2 |
| Genetics RNA 1025 | | | | | |
| Liver Cancer Research | 4.9 | 6.4 | 7.8 | 6.5 | 10.7 |
| Genetics RNA 1026 | | | | | |
| Paired Liver Cancer Tissue | 4.2 | 3.0 | 5.0 | 9.9 | 5.2 |
| Research Genetics RNA | | | | | |
| 6004-T | | | | | |
| Paired Liver Tissue | 3.5 | 4.2 | 4.7 | 7.9 | 3.7 |
| Research Genetics RNA | | | | | |
| 6004-N | | | | | |
| Paired Liver Cancer Tissue | 8.2 | 10.3 | 7.9 | 11.5 | 6.7 |
| Research Genetics RNA | | | | | |
| 6005-T | | | | | |
| Paired Liver Tissue | 2.7 | 1.6 | 2.0 | 3.2 | 2.3 |
| Research Genetics RNA | | | | | |
| 6005-N | | | | | |
| Normal Bladder GENPAK | 13.6 | 11.5 | 6.8 | 17.9 | 15.2 |
| 061001 | | | | | |
| Bladder Cancer Research | 14.5 | 14.2 | 10.7 | 22.8 | 14.2 |
| Genetics RNA 1023 | | | | | |
| Bladder Cancer | 22.7 | 17.7 | 18.0 | 29.3 | 23.5 |
| INVITROGEN A302173 | | | | | |
| 87071 Bladder Cancer | 26.1 | 21.0 | 14.5 | 29.3 | 28.3 |
| (OD04718-01) | | | | | |
| 87072 Bladder Normal | 3.1 | 3.2 | 2.9 | 5.0 | 4.2 |
| Adjacent (OD04718-03) | | | | | |
| Normal Ovary Res. Gen. | 3.6 | 4.6 | 1.4 | 4.7 | 5.4 |
| Ovarian Cancer GENPAK | 89.5 | 100.0 | 40.9 | 100.0 | 76.3 |
| 064008 | | | | | |
| 87492 Ovary Cancer | 16.7 | 15.6 | 9.7 | 43.2 | 19.5 |
| (OD04768-07) | | | | | |
| 87493 Ovary NAT | 10.8 | 6.7 | 6.5 | 7.9 | 8.3 |
| (OD04768-08) | | | | | |
| Normal Stomach GENPAK | 14.7 | 14.8 | 11.8 | 39.5 | 13.1 |
| 061017 | | | | | |
| Gastric Cancer Clontech | 2.9 | 2.8 | 1.4 | 6.0 | 2.9 |
| 9060358 | | | | | |
| NAT Stomach Clontech | 7.4 | 10.8 | 6.4 | 19.9 | 8.7 |
| 9060359 | | | | | |
| Gastric Cancer Clontech | 21.6 | 21.2 | 11.1 | 58.6 | 32.3 |
| 9060395 | | | | | |
| NAT Stomach Clontech | 23.7 | 13.8 | 6.8 | 34.6 | 22.2 |
| 9060394 | | | | | |
| Gastric Cancer Clontech | 24.8 | 25.2 | 15.4 | 78.5 | 31.9 |
| 9060397 | | | | | |
| NAT Stomach Clontech | 6.1 | 7.5 | 3.9 | 14.5 | 7.9 |
| 9060396 | | | | | |
| Gastric Cancer GENPAK | 7.0 | 7.3 | 2.5 | 14.8 | 13.0 |
| 064005 | | | | | |

TABLE 62
Panel 3D

| | Relative | | Relative |
|---|---|---|---|
| | Expression(%) | | Expression(%) |
| | 3dx4tm6021t_ | | 3dx4tm6021t_ |
| Tissue Name | ag2263_b1 | Tissue Name | ag2263_b1 |
| 94905_Daoy_Medulloblastoma/ | 19.1 | 94954_Ca Ski _Cervical | 0.4 |
| Cerebellum_sccDNA | | epidermoid carcinoma | |
| | | (metastasis)_sscDNA | |
| 94906_TE671_Medulloblastom/ | 8.4 | 94955_ES-2_Ovarian clear cell | 0.0 |
| Cerebellum_sscDNA | | carcinoma_sscDNA | |
| 94907_D283 | 39.3 | 94957_Ramos/6 h stim_{—} | 0.0 |
| Med_Medulloblastoma/ | | Stimulated with PMA/ionomycin | |
| Cerebellum_sscDNA | | 6h_sscDNA | |
| 94908_PFSK-1_Primitive | 59.5 | 94958_Ramos/14 h stim_{—} | 0.0 |
| Neuroectodermal/Cerebellum_ss | | Stimulated with PMA/ionomycin | |
| cDNA | | 14 h_sscDNA | |
| 94909_XF-498_CNS_{—} | 0.9 | 94962_MEG-01_Chronic | 3.9 |
| sscDNA | | myelogenous leukemia | |
| | | (megokaryoblast)_sscDNA | |
| 94910_SNB- | 35.4 | 94963_Raji_Burkitt's | 0.0 |
| 78_CNS/glioma_sscDNA | | lymphoma_sscDNA | |
| 94911_SF-. | 0.0 | 94964_Daudi_Burkitt's | 0.0 |
| 268_CNS/glioblastoma_sscDNA | | lymphoma_sscDNA | |
| 94912_T98G_Glioblastoma_{—} | 1.2 | 94965_U266_B-cell | 0.0 |
| DNA | | plasmacytoma/myeloma_sscDNA | |
| 96776_8K-N- | 94.4 | 94968_CA46_Burkitt's | 0.0 |
| SH_Neuroblastoma | | lymphoma_sscDNA | |
| (metastasis)_sscDNA | | | |
| 94913_SF- | 0.3 | 94970_RL_non-Hodgkin's B- | 0.0 |
| 295_CNS/glioblastoma_sscDNA | | cell lymphoma_sscDNA | |
| 94914_Cerebellum_sscDNA | 37.4 | 94972_JM1_pre-B-cell | 0.0 |
| | | lymphoma/leukemia_sscDNA | |
| 96777_Cerebellum_sscDNA | 35.1 | 94973_Jurkat_T cell | 0.5 |
| | | leukemia_sscDNA | |
| 94916_NCI- | 4.3 | 94974_TF- | 73.1 |
| H292_Mucoepidermoid lung | | 1_Erythroleukemia_sscDNA | |
| carcinoma_sscDNA | | | |
| 94917_DMS-114_Small cell | 6.6 | 94975_HUT 78_T-cell | 0.0 |
| lung cancer_sscDNA | | lymphoma_sscDNA | |
| 94918_DMS-79_Small cell lung | 100.0 | 94977_U937_Histiocytic | 0.0 |
| cancer/neuroendocrine_sscDNA | | lymphoma_sscDNA | |
| 94919_NCI-H146_Small cell | 37.3 | 94980_KU-812_Myelogenous | 0.6 |
| lung | | leukemia_sscDNA | |
| cancer/neuroendocrine sscDNA | | | |
| 94920_NCI-H526_Small cell | 17.2 | 94981_769-P_Clear cell renal | 0.0 |
| lung | | carcinoma_sscDNA | |
| cancer/neuroendocrine_sscDNA | | | |
| 94921_NCI-N417_Small cell | 88.8 | 94983_Caki-2_Clear cell renal | 0.0 |
| lung | | carcinoma_sscDNA | |
| cancer/neuroendocrine_sscDNA | | | |
| 94923_NCI-H82_Small cell lung | 95.3 | 94984_SW 839_Clear cell renal | 0.0 |
| cancer/neuroendocrine_sscDNA | | carcinoma_sscDNA | |
| 94924_NCI-H157_Squamous | 0.8 | 94986_G401_Wilms' | 2.8 |
| cell lung cancer | | tumor_sscDNA | |
| (metastasis)_sscDNA | | | |
| 94925_NCI-H1155_Large cell | 55.7 | 94987_Hs766T_Pancreatic | 0.6 |
| lung | | carcinoma (LN | |
| cancer/neuroendocrine_sscDNA | | metastasis)_sscDNA | |
| 94926_NCI-H1299_Large cell | 0.8 | 94988_CAPAN-1_Pancreatic | 3.1 |
| lung | | adenocarcinoma (liver | |
| cancer/neuroendocrine_sscDNA | | metastasis)_sscDNA | |
| 94927_NCI-H727_Lung | 0.7 | 94989_SU86.86_Pancreatic | 0.4 |
| carcinoid_sscDNA | | carcinoma (liver | |
| | | metastasis)_sscDNA | 0.4 |
| 94928_NCI-UMC-11_Lung | 7.9 | 94990_BxPC-3_Pancreatic | 22.9 |
| carcinoid_sscDNA | | adenocarcinoma_sscDNA | |
| 94929_LX-1_Small cell lung | 1.8 | 94991_HPAC_Pancreatic | 35.7 |
| cancer_sscDNA | | adenocarcinoma_sscDNA | |
| 94930_Colo-205_Colon | 0.3 | 94992_MIA PaCa-2_Pancreatic | 0.6 |
| cancer_sscDNA | | carcinoma_sscDNA | |
| 94931_KM12_Colon | 0.1 | 94993_CFPAC-1_Pancreatic | 1.1 |
| cancer_sscDNA | | ductal adenocarcinoma_sscDNA | |
| 94932_KM20L2_Colon | 0.6 | 94994_PANC-1_Pancreatic | 0.3 |
| cancer_sscDNA | | epithelioid ductal | |
| | | carcinoma_sscDNA | |
| 94933_NCI-H716_Colon | 70.2 | 94996_T24_Bladder carcinma | 0.0 |
| cancer_sscDNA | | (transitional cell)_sscDNA | |
| 94935_SW-48_Colon | 0.0 | 94997_5637_Bladder | 2.2 |
| adenocarcinoma_sscDNA | | carcinoma_sscDNA | |
| 94936_SW1116_Colon | 16.6 | 94998_HT-1197_Bladder | 0.4 |
| adenocarcinoma_sscDNA | | carcinoma_sscDNA | |
| 94937_LS 174T_Colon | 4.2 | 94999_UM-UC-3_Bladder | 0.2 |
| adenocarcinoma_sscDNA | | carcinma (transitional | |
| | | cell)_sscDNA | |
| 94938_SW-948_Colon | 0.4 | 95000_A204_Rhabdomyosarcoma_{—} | 0.0 |
| adenocarcinoma_sscDNA | | sscDNA | |
| 94939_SW-480_Colon | 0.0 | 95001_HT- | 7.9 |
| adenocarcinoma_sscDNA | | 1080_Fibrosarcoma_sscDNA | |
| 94940_NCI-SNU-5_Gastric | 1.8 | 95002_MG-63_Osteosarcoma | 16.3 |
| carcinoma_sscDNA | | (bone)_sscDNA | |
| 94941_KATO III_Gastric | 17.5 | 95003_SK-LMS- | 0.0 |
| carcinoma_sscDNA | | 1_Leiomyosarcoma | |
| | | (vulva)_sscDNA | |
| 94943_NCI-SNU-16_Gastric | 0.7 | 95004_SJRH30_Rhabdomyosar- | 0.0 |
| carcinoma_sscDNA | | coma (met to bone | |
| | | marrow)_sscDNA | |
| 94944_NCI-SNU-1_Gastric | 23.0 | 95005_A431_Epidermoid | 34.9 |
| carcinoma_sscDNA | | carcinoma_sscDNA | |
| 94946_RF-1_Gastric | 0.0 | 95007_WM266- | 0.0 |
| adenocarcinoma_sscDNA | | 4_Melanoma_sscDNA | |
| 94947_RF-48_Gastric | 0.0 | 95010_DU 145_Prostate | 0.0 |
| adenocarcinoma_sscDNA | | carcinoma (brain | |
| | | metastasis)_sscDNA | |
| 96778_MKN-45_Gastric | 11.5 | 95012_MDA-MB-468_Breast | 16.3 |
| carcinoma_sscDNA | | adenocarcinoma_sscDNA | |
| 94949_NCI-N87_Gastric | 24.1 | 95013_SCC-4 Squamous cell | 0.0 |
| carcinoma_sscDNA | | carcinoma of tongue_sscDNA | |
| 94951_OVCAR-5_Ovarian | 3.7 | 95014_SCC-9_Squamous cell | 0.0 |
| carcinoma_sscDNA | | carcinoma of tongue_sscDNA | |
| 94952_RL95-2_Uterine | 4.6 | 95015_SCC-15_Squamous cell | 0.0 |
| carcinoma_sscDNA | | carcinoma of tongue_sscDNA | |
| 94953_HelaS3_Cervical | 5.9 | 95017_CAL 27_Squamous cell | 7.1 |
| adenocarcinoma_sscDNA | | carcinoma of tongue_sscDNA | |

TABLE 63
Panel 4D

| | Relative | Relative | Relative | Relative |
|---|---|---|---|---|
| | Expression (%) | Expression (%) | Expression (%) | Expression (%) |
| | 4dtm2473t_{—} | 4dtm3214t_{—} | 4dtm4353t_{—} | 4dtm4222_{—} |
| Tissue Name | ag1522 | ag2263 | ag1848 | ag2422 |
| 93768_Secondary Th1_anti- | 0.0 | 0.0 | 0.1 | 0.2 |
| CD28/anti-CD3 | | | | |
| 93769_Secondary Th2_anti- | 0.0 | 0.0 | 0.0 | 0.0 |
| CD28/anti-CD3 | | | | |
| 93770_Secondary Tr1_anti- | 0.0 | 0.0 | 0.0 | 4.6 |
| CD28/anti-CD3 | | | | |
| 93573_Secondary | 0.1 | 0.1 | 0.0 | 0.0 |
| Th1_resting day 4-6 in IL-2 | | | | |
| 93572_Secondary | 0.0 | 0.0 | 0.0 | 0.0 |
| Th2_resting day 4-6 in IL-2 | | | | |
| 93571_Secondary | 0.0 | 0.0 | 0.0 | 0.2 |
| Tr1_resting day 4-6 in IL-2 | | | | |
| 93568_primary Th1_anti- | 0.1 | 0.2 | 0.2 | 1.0 |
| CD28/anti-CD3 | | | | |
| 93569_primary Th2_anti- | 0.1 | 0.1 | 0.2 | 0.3 |
| CD28/anti-CD3 | | | | |
| 93570_primary Tr1_anti- | 0.2 | 0.0 | 0.5 | 0.6 |
| CD28/anti-CD3 | | | | |
| 93565_primary Th1_resting | 0.0 | 0.0 | 0.0 | 0.0 |
| dy 4-6 in IL-2 | | | | |
| 93566_primary Th2_resting | 0.0 | 0.0 | 0.0 | 0.0 |
| dy 4-6 in IL-2 | | | | |
| 93567_primary Tr1_resting | 0.0 | 0.0 | 0.0 | 0.0 |
| dy 4-6 in IL-2 | | | | |
| 93351_CD45RA CD4 | 4.9 | 8.5 | 6.3 | 10.6 |
| lymphocyte_anti-CD28/anti- | | | | |
| CD3 | | | | |
| 93352_CD45RO CD4 | 0.0 | 0.0 | 0.0 | 0.0 |
| lymphocyte_anti-CD28/anti- | | | | |
| CD3 | | | | |
| 93251_CD8 | 0.0 | 0.0 | 0.0 | 0.0 |
| Lymphocytes_anti- | | | | |
| CD28/anti-CD3 | | | | |
| 93353_chronic CD8 | 0.0 | 0.0 | 0.0 | 0.0 |
| Lymphocytes 2ry_resting dy | | | | |
| 4—6 in IL-2 | | | | |
| 93574_chronic CD8 | 0.0 | 0.0 | 0.0 | 0.0 |
| Lymphocytes 2ry_activated | | | | |
| CD3/CD28 | | | | |
| 93354_CD4 none | 0.0 | 0.0 | 0.0 | 0.0 |
| 93252_Secondary | 0.0 | 0.0 | 0.0 | 0.0 |
| Th1/Th2/Tr1_anti-CD95 | | | | |
| CH11 | | | | |
| 93103_LAK cells_resting | 1.8 | 2.0 | 2.7 | 5.8 |
| 93788_LAK cells_IL-2 | 0.0 | 0.0 | 0.0 | 0.0 |
| 93787_LAK cells_IL-2 + IL- | 0.0 | 0.0 | 0.0 | 0.2 |
| 12 | | | | |
| 93789_LAK cells_IL-2 + IFN | 0.0 | 0.0 | 0.0 | 0.2 |
| gamma | | | | |
| 93790_LAK cells_IL-2 + IL- | 0.0 | 0.0 | 0.4 | 0.1 |
| 18 | | | | |
| 93104_LAK | 1.1 | 1.7 | 1.0 | 2.5 |
| cells_PMA/ionomycin and | | | | |
| IL-18 | | | | |
| 93578_NK Cells IL-2_{—} | 0.0 | 0.0 | 0.1 | 0.0 |
| resting | | | | |
| 93109_Mixed Lymphocyte | 0.0 | 0.2 | 0.0 | 0.2 |
| Reaction_Two Way MLR | | | | |
| 93110_Mixed Lymphocyte | 0.2 | 0.8 | 0.3 | 0.6 |
| Reaction_Two Way MLR | | | | |
| 93111_Mixed Lymphocyte | 0.5 | 0.1 | 0.2 | 0.3 |
| Reaction_Two Way MLR | | | | |
| 93112_Mononuclear Cells | 0.0 | 0.1 | 0.0 | 0.0 |
| (PBMCs)_resting | | | | |
| 93113_Mononuclear Cells | 0.0 | 0.0 | 0.1 | 0.0 |
| (PBMCs)_PWM | | | | |
| 93114_Mononuclear Cells | 0.0 | 0.0 | 0.0 | 0.0 |
| (PBMCs)_PHA-L | | | | |
| 93249_Ramos (B cell)_none | 0.0 | 0.0 | 0.0 | 0.0 |
| 93250_Ramos (B | 0.0 | 0.0 | 0.0 | 0.0 |
| cell)_ionomycin | | | | |
| 93349_B | 0.2 | 0.0 | 0.0 | 0.0 |
| lymphocytes_PWM | | | | |
| 93350_B | 0.0 | 0.1 | 0.0 | 0.3 |
| lymphoytes_CD40L and IL-4 | | | | |
| 92665_EOL-1 | 0.2 | 0.4 | 0.2 | 0.0 |
| (Eosinophil)_dbcAMP | | | | |
| differentiated | | | | |
| 93248_EOL-1 | 0.0 | 0.2 | 0.4 | 0.6 |
| (Eosinophil)_dbcAMP/PMA | | | | |
| ionomycin | | | | |
| 93356_Dendritic Cells_none | 1.4 | 1.0 | 1.1 | 2.8 |
| 93355_Dendritic Cells_LPS | 0.3 | 0.3 | 0.4 | 0.4 |
| 100 ng/ml | | | | |
| 93775_Dendritic Cells_anti- | 2.4 | 3.5 | 3.0 | 6.7 |
| CD40 | | | | |
| 93774_Monocytes_resting | 0.8 | 0.6 | 0.8 | 1.3 |
| 93776_Monocytes_LPS | 0.0 | 0.3 | 0.0 | 0.0 |
| 50 ng/ml | | | | |
| 93581_Macrophages_resting | 1.3 | 0.0 | 1.0 | 2.0 |
| 93582 _Macrophages_LPS | 0.0 | 0.1 | 0.2 | 0.4 |
| 100 ng/ml | | | | |
| 93098_HUVEC | 1.1 | 0.6 | 1.4 | 2.5 |
| (Endothelial)_none | | | | |
| 93099_HUVEC | 4.4 | 2.9 | 4.7 | 6.0 |
| (Endothelial)_starved | | | | |
| 93100_HUVEC | 1.7 | 1.0 | 2.8 | 2.3 |
| (Endothelial)_IL-1b | | | | |
| 93779_HUVEC | 1.6 | 2.5 | 1.4 | 1.9 |
| (Endothelial)_IFN gamma | | | | |
| 93102_HUVEC | 0.3 | 0.5 | 0.3 | 0.5 |
| (Endothelial)_TNF alpha + | | | | |
| IFN gamma | | | | |
| 93101_HUVEC | 0.2 | 0.3 | 0.3 | 1.3 |
| (Endothelial)_TNF alpha + | | | | |
| IL4 | | | | |
| 93781_HUVEC | 0.9 | 2.2 | 1.2 | 0.5 |
| (Endothelial)_IL-11 | | | | |
| 93583_Lung Microvascular | 2.2 | 2.8 | 6.5 | 6.7 |
| Endothelial Cells_none | | | | |
| 93584_Lung Microvascular | 12.7 | 8.5 | 11.9 | 15.5 |
| Endothelial Cells_TNFa | | | | |
| (4 ng/ml) and IL1b (1 ng/ml) | | | | |
| 92662_Microvascular | 32.1 | 22.4 | 30.8 | 22.4 |
| Dermal endothelium_none | | | | |
| 92663_Microsvasular | 16.3 | 8.8 | 16.2 | 14.4 |
| Dermal endothelium_TNFa | | | | |
| (4 ng/ml) and IL1b (1 ng/ml) | | | | |
| 93773_Bronchial | 24.0 | 15.1 | 31.2 | 50.7 |
| epithelium_TNFa (4 ng/ml) | | | | |
| and IL1b (1 ng/ml)** | | | | |
| 93347_Small Airway | 8.8 | 6.7 | 5.9 | 12.8 |
| Epithelium_none | | | | |
| 93348_Small Airway | 31.9 | 21.0 | 43.5 | 44.8 |
| Epithelium_TNFa (4 ng/lml) | | | | |
| and IL1b (1 ng/ml) | | | | |
| 92668_Coronery Artery | 27.4 | 8.5 | 28.7 | 35.8 |
| SMC_resting | | | | |
| 92669_Coronery Artery | 12.9 | 27.4 | 21.6 | 17.8 |
| SMC_TNFa (4 ng/ml) and | | | | |
| IL1b (1 ng/ml) | | | | |
| 93107_astrocytes_resting | 17.1 | 23.8 | 14.9 | 24.3 |
| 93108_astrocytes_TNFa | 32.8 | 28.1 | 29.5 | 35.1 |
| (4 ng/ml) and IL1b (1 ng/ml) | | | | |
| 92666_KU-812 | 1.0 | 1.3 | 1.8 | 0.7 |
| (Basophil)_resting | | | | |
| 92667_KU-812 | 1.4 | 2.0 | 3.3 | 3.7 |
| (Basophil)_PMA/ionoycin | | | | |
| 93579_CCD1106 | 1.4 | 0.7 | 0.2 | 2.7 |
| (Keratinocytes)_none | | | | |
| 93580_CCD1106 | 0.9 | 0.8 | 0.3 | 1.3 |
| (Keratinocytes)_TNFa and | | | | |
| IFNg** | | | | |
| 93791_Liver Cirrhosis | 2.9 | 2.4 | 3.0 | 4.8 |
| 93792_Lupus Kidney | 3.0 | 0.9 | 2.9 | 4.4 |
| 93577_NCI-H292 | 10.4 | 5.6 | 13.7 | 18.8 |
| 93358_NCI-H292_IL-4 | 14.2 | 6.8 | 14.9 | 17.1 |
| 93360_NCI-H292_IL-9 | 13.2 | 9.3 | 16.7 | 12.8 |
| 93359_NCI-H292_IL-13 | 9.4 | 15.9 | 8.6 | 9.0 |
| 93357_NCI-H292_IFN | 3.8 | 4.7 | 4.7 | 5.3 |
| gamma | | | | |
| 93777_HPAEC_- | 1.2 | 1.6 | 1.0 | 2.8 |
| 93778_HPAEC_IL-1 | 5.8 | 4.7 | 2.6 | 6.0 |
| beta/TNA alpha | | | | |
| 93254_Normal Human Lung | 100.0 | 100.0 | 100.0 | 100.0 |
| Fibroblast_none | | | | |
| 93253_Normal Human Lung | 8.5 | 15.9 | 12.2 | 15.2 |
| Fibroblast_TNFa (4 ng/ml) | | | | |
| and IL-1b (1 ng/ml) | | | | |
| 93257_Normal Human Lung | 74.2 | 45.7 | 79.6 | 97.3 |
| Fibroblast_IL-4 | | | | |
| 93256_Normal Human Lung | 27.7 | 30.6 | 48.6 | 50.3 |
| Fibroblast_IL-9 | | | | |
| 93255_Normal Human Lung | 48.0 | 27.4 | 39.5 | 55.9 |
| Fibroblast_IL-13 | | | | |
| 93258_Normal Human Lung | 76.3 | 42.6 | 82.9 | 98.6 |
| Fibroblast_IFN gamma | | | | |
| 93106_Dermal Fibroblasts | 52.8 | 27.2 | 56.3 | 65.5 |
| CCD1070_resting | | | | |
| 93361_Dermal Fibroblasts | 33.9 | 19.8 | 42.6 | 46.7 |
| CCD1070_TNF alpha 4 ng/ml | | | | |
| 93105_Dermal Fibroblasts | 29.1 | 70.2 | 27.9 | 28.9 |
| CCD1070_IL-1 beta 1 ng/ml | | | | |
| 93772_dermal | 6.1 | 8.9 | 3.6 | 7.9 |
| fibroblast_IFN gamma | | | | |
| 93771_dermal | 14.5 | 17.3 | 16.2 | 18.9 |
| fibroblast_IL-4 | | | | |
| 93260_IBD Colitis 2 | 0.1 | 0.2 | 0.1 | 0.5 |
| 93261_IBD Crohns | 0.6 | 0.0 | 0.4 | 0.8 |
| 735010_Colon_normal | 7.6 | 8.0 | 6.4 | 11.3 |
| 735019_Lung_none | 59.5 | 47.6 | 75.8 | 74.7 |
| 64028-1_Thymus_none | 16.5 | 10.2 | 17.3 | 19.6 |
| 64030-1_Kidney_none | 6.8 | 3.0 | 9.0 | 6.5 |

TABLE 64
Panel CNS_1

| | Relative | | Relative |
|---|---|---|---|
| | Expression (%) | | Expression (%) |
| | CNS1tm6191t_{—} | | CNS1tm6191t_{—} |
| Tissue Name | ag2263_a2 | Tissue Name | ag2263_a2 |
| 102633_BA4 Control | 22.8 | 102605_BA17 PSP | 11.2 |
| 102641_BA4 Control2 | 38.1 | 102612_BA17 PSP2 | 7.1 |
| 102625_BA4 Alzheimer's2 | 3.7 | 102637_Sub Nigra Control | 100.0 |
| 102649_BA4 Parkinson's | 45.6 | 102645_Sub Nigra Control2 | 51.6 |
| 102656_BA4 Parkinson's2 | 31.1 | 102629_Sub Nigra Alzheimer's2 | 30.7 |
| 102664_BA4 Huntington's | 12.3 | 102660_Sub Nigra Parkinson's2 | 89.0 |
| 102671_BA4 Huntington's2 | 12.2 | 102667_Sub Nigra Huntington's | 58.9 |
| 102603_BA4 PSP | 13.6 | 102674_Sub Nigra Huntington's2 | 16.0 |
| 102610_BA4 PSP2 | 42.5 | 102614_Sub Nigra PSP2 | 22.4 |
| 102588_BA4 Depression | 27.8 | 102592_Sub Nigra Depression | 40.4 |
| 102596_BA4 Depression2 | 10.8 | 102599_Sub Nigra Depression2 | 12.7 |
| 102634_BA7 Control | 28.3 | 102636_Glob Palladus Control | 36.0 |
| 102642_BA7 Control2 | 27.2 | 102644_Glob Palladus Control2 | 21.2 |
| 102626_BA7 Alzheimer's2 | 5.5 | 102620_Glob Palladus Alzheimer's | 26.0 |
| 102650_BA7 Parkinson's | 13.2 | 102628_Glob Palladus | 11.1 |
| | | Alzheimer's2 | |
| 102657_BA7 Parkinson's2 | 12.7 | 102652_Glob Palladus Parkinson's | 73.0 |
| 102665_BA7 Huntington's2 | 14.7 | 102659_Glob Palladus | 15.6 |
| | | Parkinson's2 | |
| 102672_BA7 Huntington's2 | 22.2 | 102606_Glob Palladus PSP | 14.9 |
| 102604_BA7 PSP | 28.9 | 102613_Glob Palladus PSP2 | 10.4 |
| 102611_BA7 PSP2 | 8.9 | 102591_Glob Palladus Depression | 28.3 |
| 102589_BA7 Depression | 5.4 | 102638_Temp Pole Control | 5.4 |
| 102632_BA9 Control | 14.2 | 102646_Temp Pole Control2 | 25.0 |
| 102640_BA9 Control2 | 56.8 | 102622_Temp Pole Alzheimer's | 10.0 |
| 102617_BA9 Alzheimer's | 5.5 | 102630_Temp Pole Alzheimer's2 | 2.5 |
| 102624_BA9 Alzheimer's2 | 13.7 | 102653_Temp Pole Parkinson's | 15.5 |
| 102648_BA9 Parkinson's | 16.1 | 102661_Temp Pole Parkinson's2 | 27.9 |
| 102655_BA9 Parkinson's2 | 21.0 | 102668_Temp Pole Huntington's | 22.2 |
| 102663_BA9 Huntington's | 21.3 | 102607_Temp Pole PSP | 1.3 |
| 102670_BA9 Huntington's2 | 11.9 | 102615_Temp Pole PSP2 | 6.3 |
| 102602_BA9 PSP | 27.7 | 102600_Temp Pole Depression2 | 12.3 |
| 102609_BA9 PSP2 | 5.9 | 102639_Cing Gyr Control | 48.1 |
| 102587_BA9 Depression | 11.0 | 102647_Cing Gyr Control2 | 28.0 |
| 102595_BA9 Depression2 | 9.5 | 102623_Cing Gyr Alzheimer's | 27.1 |
| 102635_BA17 Control | 24.8 | 102631_Cing Gyr Alzheimer's2 | 13.1 |
| 102643_BA17 Control2 | 45.4 | 102654_Cing Gyr Parkinson's | 29.5 |
| 102627_BA17 Alzheimer's2 | 6.4 | 102662_Cing Gyr Parkinson's2 | 37.2 |
| 102651_BA17 Parkinson's | 35.2 | 102669_Cing Gyr Huntington's | 70.3 |
| 102658_BA17 Parkinson's2 | 15.2 | 102676_Cing Gyr Huntington's2 | 32.0 |
| 102666_BA17 Huntington's | 15.5 | 102608_Cing Gyr PSP | 42.6 |
| 102673_BA17 Huntington's2 | 8.1 | 102616_Cing Gyr PSP2 | 8.3 |
| 102590_BA17 Depression | 26.1 | 102594_Cing Gyr Depression | 20.5 |
| 102597_BA17 Depression2 | 59.7 | 102601_Cing Gyr Depression2 | 36.1 |

TABLE 65
Panel CNS_Neurodegeneration_v1.0

| | Relative | Relative |
|---|---|---|
| | Expression (%) | Expression (%) |
| | tm6993t_{—} | tm6900f_{—} |
| Tissue Name | ag1848_b2 | ag2422_b2s2 |
| AD 1 Hippo | 28.3 | 21.2 |
| AD 2 Hippo | 38.0 | 38.5 |
| AD 3 Hippo | 12.0 | 14.8 |
| AD 4 Hippo | 17.7 | 13.3 |
| AD 5 hippo | 45.4 | 57.7 |
| AD 6 Hippo | 66.9 | 95.3 |
| Control 2 Hippo | 43.3 | 46.0 |
| Control 4 Hippo | 34.1 | 30.2 |
| Control (Path) 3 Hippo | 3.9 | 12.6 |
| AD 1 Temporal Ctx | 47.1 | 40.4 |
| AD 2 Temporal Ctx | 49.2 | 39.6 |
| AD 3 Temporal Ctx | 14.5 | 15.6 |
| AD 4 Temporal Ctx | 41.4 | 36.2 |
| AD 5 Inf Temporal Ctx | 78.1 | 88.4 |
| AD 5 Sup Temporal Ctx | 40.9 | 56.7 |
| AD 6 Inf Temporal Ctx | 83.9 | 74.1 |
| AD 6 Sup Temporal Ctx | 58.2 | 71.5 |
| Control 1 Temporal Ctx | 17.9 | 11.3 |
| Control 2 Temporal Ctx | 45.7 | 44.7 |
| Control 3 Temporal Ctx | 14.6 | 15.6 |
| Control 4 Temporal Ctx | 23.2 | 19.0 |
| Control (Path) 1 Temporal Ctx | 45.9 | 40.1 |
| Control (Path) 2 Temporal Ctx | 24.7 | 21.7 |
| Control (Path) 3 Temporal Ctx | 6.0 | 7.7 |
| Control (Path) 4 Temporal Ctx | 32.0 | 23.9 |
| AD 1 Occipital Ctx | 24.2 | 26.4 |
| AD 2 Occipital Ctx (Missing) | 0.0 | 0.0 |
| AD 3 Occipital Ctx | 19.2 | 18.1 |
| AD 4 Occipital Ctx | 30.1 | 23.2 |
| AD 5 Occipital Ctx | 6.0 | 26.7 |
| AD 6 Occipital Ctx | 43.3 | 50.2 |
| Control 1 Occipital Ctx | 14.5 | 12.7 |
| Control 2 Occipital Ctx | 66.7 | 76.0 |
| Control 3 Occipital Ctx | 17.8 | 17.4 |
| Control 4 Occipital Ctx | 23.3 | 15.7 |
| Control (Path) 1 Occipital Ctx | 100.0 | 100.0 |
| Control (Path) 2 Occipital Ctx | 18.7 | 12.3 |
| Control (Path) 3 Occipital Ctx | 7.9 | 7.1 |
| Control (Path) 4 Occipital Ctx | 24.4 | 13.9 |
| Control 1 Parietal | 23.2 | 22.2 |
| Control 2 Parietal | 46.0 | 64.3 |
| Control 3 Parietal | 26.1 | 17.2 |
| Control (Path) 1 Parietal | 51.1 | 54.1 |
| Control (Path) 2 Parietal | 36.4 | 27.8 |
| Control (Path) 3 Parietal | 6.1 | 5.1 |
| Control (Path) 4 Parietal | 46.0 | 36.4 |

Panel 1.2 Summary: Ag1522 Expression of the NOV10 gene is highest in CNS cancer cell lines (CT=26.1). Of nine tissue samples derived from CNS cancer cell lines, expression of the NOV10 gene occurs in all samples, with expression high (CT=26.1, 26.6, 27.6) in three samples, moderate in five samples and low in one sample. High expression is also detectable in melanoma cell lines (CT=27.9). Significant expression of the NOV10 gene is seen in gastric cancer (28.1) and all ten samples of lung cancer cell lines in this sample. Thus, expression of the NOV10 gene could be used to distinguish those cancer cell lines from normal tissues. In addition, therapeutic modulation of the expression, or activity of the NOV10 gene product, might be of use in the treatment of melanoma, gastric cancer, lung cancer and brain cancer.

Panel 1.3D Summary Ag1522/Ag1848/Ag2263/Ag2422 Four experiments using different probe/primer sets on the same tissue panel produce results that are in excellent agreement. In all four experiments, highest expression of the NOV10 gene is detected in CNS cancer cell lines. Expression is also significant in lung cancer and melanoma cell lines and in healthy brain tissue from the hippocampus and thalamus regions. Thus, the expression of the NOV10 gene could be used to distinguish these tissue samples from other samples. Moreover, therapeutic modulation of the expression, or function, of the NOV10 gene, through the use of small molecule drugs or antibodies, might be beneficial in the treatment of melanoma, lung cancer and brain cancer.

Among metabolic tissues, there is high expression of the NOV10 gene in adult heart tissue (CT=27.8) and moderate expression in fetal heart, adult and fetal liver, pancreas, adrenal gland, thyroid and pituitary. The NOV10 gene appears to be differentially expressed in fetal (CT value=31) and adult skeletal muscle (CT value=37) using the probe and primer set Ag1848 and may be useful for the differentiation of the adult from the fetal phenotype in this tissue.

Panel 2D Summary Ag1522/Ag1848/Ag2263/Ag2422 Results from multiple experiments with four different probe and primer sets are in very good agreement. In all four experiments, highest expression of the NOV10 gene is detected in thyroid and ovarian cancers (CTs=27-30), with lower expression also seen in most of the other tissues on this panel. Thus, the expression of the NOV10 gene could be used to distinguish ovarian and thyroid cancer cell lines from other tissues. Moreover, therapeutic modulation of the expression this gene, or its function, through the use of small molecule drugs or antibodies, might be of benefit in the treatment of ovarian and thyroid cancer. In addition, experiments with Ag2263 show differential expression between samples derived from lung cancer and their adjacent normal tissues. Thus, expression of the NOV10 gene could be used to distinguish cancerous lung tissue from normal lung tissue. Moreover, therapeutic modulation of the expression or function of this gene or its protein product, through the use of antibodies or small molecule drugs, might be of benefit in the treatment of lung cancer

Panel 3D Summary Ag2263 Expression of the NOV10 gene occurs at moderate levels across all the tissues in this panel. Highest expression is detected in a small cell lung cancer (CT=30.6) and neuroblastoma (CT=30.7). In addition, significant expression is detected in a cluster of small cell lung cancer lines. Thus, this gene could be used to distinguish lung cancer cell lines from other samples. Moreover, therapeutic modulation of the NOV10 gene or its protein product, through the use of small molecule drugs or antibodies might be of benefit in the treatment of small cell lung cancer.

Panel 4D Summary Ag1522/Ag1848/Ag2263/Ag2422 Experiments using each of the four probe and primer sets that correspond to the NOV10 gene produce results that are in excellent agreement. In all the experiments, expression of the NOV10 gene occurs at moderate to low levels in many of the tissues in the sample. Highest expression in each experiment occurs in lung fibroblasts (CT=29). Moderate expression in lung fibroblasts treated with IL-4 is also consistent among all four experiments (CT=30). Lower expression is also detected in a variety of fibroblasts, endothelial and smooth muscle cells. The expression of the NOV10 gene produces a complex profile; it is upregulated by TNF-alpha in small airway epithelium, but clearly downregulated by the same stimulus in lung fibroblasts. The gene most probably encodes a netrin receptor that may be important in understanding cell migration. Regulation of the protein encoded for by the NOV10 gene could potentially control the progression of keloid formation, emphysema and other conditions in which TNF-alpha and IL-1 beta are present and tissue remodeling may occur.

Panel CNS_{—}1 Summary Ag2263 Expression of the NOV10 gene is moderate to low across many of the tissues in this panel. Highest expression is detected in the substantia nigra (CT=31.4). Although no disease-specific expression is seen in this panel, the expression profile confirms the expression of this gene in the central nervous system. Please see panel_CNS_neurodegeneration for potential utility of the NOV10 gene regarding the CNS.

Panel CNS_Neurodegeneration_v1.0 Summary Ag1848/Ag2422 Two experiments using different probe/primer sets produce results that are in good agreement. Highest expression of the NOV10 gene is detected in the occipital cortex of a control patient. Significant levels of expression are also detected in the hippocampus, inferior temporal cortex, and the superior temporal cortex of brain tissue from an Alzheimer's patient.

Based on its homology, the NOV10 gene product is most similar to an UNC5H receptor, which as a class are known to act both in axon guidance and neuronal migration during development, as well as inducers of apoptosis (except when stimulated by the ligand netrin-1). Panel CNS_Neurodegeneration_V1.0 shows a moderate increase (1.5 to 2-fold) in the temporal cortex of the Alzheimer's disease brain when compared to non-demented elderly showing a high amyloid plaque load. Thus the NOV10 gene represents a protein that differentiates demented and non-demented elderly who have a severe amyloid plaque load, making it an excellent drug target in Alzheimer's disease. The modulation and/or selective stimulation of this receptor may be of use in enhancing or directing compensatory synatogenesis and axon/dendritic outgrowth in response to neuronal death (stroke, head trauma) neurodegeneration (Alzheimer's, Parkinson's, Huntington's, spinocerebellar ataxia, progressive supranuclear palsy) or spinal cord injury. Furthermore, antagonism of this receptor may decrease apoptosis in Alzheimer's disease. (Ellezam et al., Exp Neurol. 168:105-15, 2001; Braisted et al., J Neurosci. 20:5792-801, 2000; Montell, Development 126:3035-46, 1999.)

NOV11a: Hepatocyte Growth Factor-like

Expression of the NOV11a gene (GMba446g13_A) was assessed using the primer-probe sets Ag3086 and Ag3797, described in Tables 66 and 67. Results from RTQ-PCR runs are shown in Tables 68, 69, 70, 71, 72 and 73.

TABLE 66

Probe Name Ag3086

| | | | | | |
|---|---|---|---|---|---|
| | | | | Start | |
| Primers | Sequences | TM | Length | Position | SEQ ID NO: |

| | | | | | |
|---|---|---|---|---|---|
| Forward | 5′-GGACCCCATTCGACTACTGT-3′ | 20 | 20 | 1309 | 188 |
| Probe | FAM-5′- | 23 | 23 | 1345 | 189 |
| | CTGATGACCAGCCGCCATCAATC-3′- | | | | |
| | TAMRA | | | | |
| Reverse | 5′-TTCTCAAACTGCACCTGGTC-3′ | 20 | 20 | 1399 | 190 |

TABLE 67

Probe Name Ag3797

| | | | | | |
|---|---|---|---|---|---|
| | | | | Start | |
| Primers | Sequences | TM | Length | Position | SEQ ID NO: |

| | | | | | |
|---|---|---|---|---|---|
| Forward | 5′-TCTGGACGACAACTATTGCC-3′ | 58.7 | 20 | 627 | 191 |
| Probe | FAM-5′- | 69.2 | 25 | 672 | 192 |
| | ATGGTGCTACACTACGGATCCGCAG-3′- | | | | |
| | TAMRA | | | | |
| Reverse | 5′-GTCACAGAATTCTCGCTCGA-3′ | 59.1 | 20 | 698 | 193 |

TABLE 68
Panel 1.3D

| | Relative | | Relative |
|---|---|---|---|
| | Expression (%) | | Expression (%) |
| | 1.3dx4tm5430f_{—} | | 1.3dx4tm5430f_{—} |
| Tissue Name | ag3086_a1 | Tissue Name | ag3086_a1 |
| Liver adenocarcinoma | 0.7 | Kidney (fetal) | 31.1 |
| Pancreas | 17.9 | Renal ca. 786-0 | 0.2 |
| Pancreatic ca. CAPAN 2 | 0.6 | Renal ca. A498 | 0.5 |
| Adrenal gland | 2.7 | Renal ca. RXF 393 | 0.7 |
| Thyroid | 3.3 | Renal ca. ACHN | 0.8 |
| Salivary gland | 1.2 | Renal ca. UO-31 | 0.4 |
| Pituitary gland | 3.6 | Renal ca. TK-10 | 0.2 |
| Brain (fetal) | 3.2 | Liver | 94.2 |
| Brain (whole) | 3.4 | Liver (fetal) | 100.0 |
| Brain (amygdala) | 2.1 | Liver ca. (hepatoblast) HepG2 | 58.4 |
| Brain (cerebellum) | 1.5 | Lung | 2.8 |
| Brain (hippocampus) | 3.0 | Lung (fetal) | 12.9 |
| Brain (substantia nigra) | 1.7 | Lung ca. (small cell) LX-1 | 1.3 |
| Brain (thalamus) | 3.0 | Lung ca. (small cell) NCI-H69 | 0.2 |
| Cerebral Cortex | 0.9 | Lung ca. (s cell var.) SHP-77 | 1.2 |
| Spinal cord | 2.9 | Lung ca. (large cell) NCI-H460 | 1.4 |
| CNS ca. (glio/astro) U87-MG | 0.7 | Lung ca. (non-sm. cell) A549 | 0.2 |
| CNS ca. (glio/astro) U-118-MG | 0.9 | Lung ca. (non-s.cell) NCI-H23 | 0.9 |
| CNS ca. (astro) S5W1783 | 0.4 | Lung ca (non-s.cell) HOP-62 | 0.5 |
| CNS ca.* (neuro; met) SK-N-AS | 0.7 | Lung ca. (non-s.cl) NCI-H522 | 0.6 |
| CNS ca. (astro) SF-539 | 0.5 | Lung ca. (squam.) SW 900 | 0.4 |
| CNS ca. (astro) SNB-75 | 1.2 | Lung ca. (squam.) NCI-H596 | 0.5 |
| CNS ca. (glio) SNB-19 | 1.6 | Mammary gland | 3.1 |
| CNS ca. (glio) U251 | 2.4 | Breast ca.* (pl. effusion) MCF-7 | 0.7 |
| CNS ca. (glio) SF-295 | 0.7 | Breast ca.* (pl.ef) MDA-MB-231 | 0.7 |
| Heart (fetal) | 0.6 | Breast ca.* (pl. effusion) T47D | 2.7 |
| Heart | 0.5 | Breast ca. BT-549 | 0.7 |
| Fetal Skeletal | 0.2 | Breast ca. MDA-N | 0.0 |
| Skeletal muscle | 1.4 | Ovary | 0.4 |
| Bone marrow | 2.0 | Ovarian ca. OVCAR-3 | 0.6 |
| Thymus | 1.2 | Ovarian ca. OVCAR-4 | 0.4 |
| Spleen | 4.0 | Ovarian ca. OVCAR-5 | 0.4 |
| Lymph node | 3.1 | Ovarian ca. OVCAR-8 | 0.7 |
| Colorectal | 1.6 | Ovarian ca. IGROV-1 | 0.7 |
| Stomach | 10.3 | Ovarian ca.* (ascites) SK-OV-3 | 0.0 |
| Small intestine | 29.6 | Uterus | 3.2 |
| Colon ca. SW480 | 0.7 | Placenta | 4.6 |
| Colon ca.* (SW480 met) SW620 | 0.2 | Prostate | 2.1 |
| Colon ca. HT29 | 0.2 | Prostate ca.* (bone met) PC-3 | 0.9 |
| Colon ca. HCT-116 | 1.2 | Testis | 12.4 |
| Colon ca. CaCo-2 | 2.4 | Melanoma Hs688(A).T | 0.1 |
| 83219 CC Well to Mod Diff | 2.1 | Melanoma* (met) Hs688(B).T | 0.2 |
| (ODO3866) | | | |
| Colon ca. HCC-2998 | 1.4 | Melanoma UACC-62 | 0.4 |
| Gastric ca.* (liver met) NCI-N87 | 1.8 | Melanoma M14 | 0.8 |
| Bladder | 4.5 | Melanoma LOX IMVI | 0.0 |
| Trachea | 2.6 | Melanoma* (met) SK-MEL-5 | 0.4 |
| Kidney | 26.1 | Adipose | 2.2 |

TABLE 69
General Screening Panel_v1.4

| | Relative | | Relative |
|---|---|---|---|
| | Expression (%) | | Expression (%) |
| | 1.4x4tm7355f_{—} | | 1.4x4tm7355f_{—} |
| Tissue Name | ag3797_a1 | Tissue Name | ag3797_a1 |
| D6005-01_Human adipose | 1.4 | Renal ca._TK-10 | 28.9 |
| 112193_Metastatic melanoma | 0.4 | Bladder | 8.4 |
| 112192_Metastatic melanoma | 0.5 | Gastric ca. (liver met) NCI-N87 | 2.7 |
| 95280_Epidermis (metastatic | 0.3 | 112197_Stomach | 1.4 |
| melanoma) | | | |
| 95279_Epidermis (metastatic | 0.3 | 94938_Colon Adenocarcinoma | 1.0 |
| melanoma) | | | |
| Melanoma (met)_SK-MEL-5 | 0.5 | Colon ca._SW480 | 3.9 |
| 112196_Tongue (oncology) | 0.8 | Colon ca. (SW480 met)_SW620 | 1.2 |
| 113461_Testis Pool | 2.0 | Colon ca._HT29 | 0.2 |
| Prostate ca. (bone met)_PC-3 | 1.5 | Colon ca._HCT-116 | 4.3 |
| 113455_Prostate Pool | 1.8 | Colon ca._CaCo-2 | 11.5 |
| 103396_Placenta | 1.7 | 83219_CC Well to Mod Diff | 2.8 |
| | | (ODO3866) | |
| 113463_Uterus Pool | 0.5 | 94936_Colon Adenocarcinoma | 2.9 |
| Ovarian carcinoma_OVCAR-3 | 1.0 | 94930_Colon | 0.5 |
| Ovarian carcinoma (ascites)_SK- | 0.8 | 94935_Colon Adenocarcinoma | 0.2 |
| OV-3 | | | |
| 95297_Adenocarcinoma (ovary) | 0.3 | 113468_Colon Pool | 1.6 |
| Ovarian carcinoma_OVCAR-5 | 6.4 | 113457_Small Intestine Pool | 2.0 |
| Ovarian carcinoma_IGROV-1 | 4.6 | 113460_Stomach Pool | 1.9 |
| Ovarian carcinoma_OVCAR-8 | 2.9 | 113467_Bone Marrow Pool | 0.4 |
| 103368_Ovary | 1.9 | 103371_Fetal Heart | 0.8 |
| MCF7_breast carcinoma (pleural | 2.3 | 113451_Heart Pool | 0.7 |
| effusion) | | | |
| Breast ca. (pleural | 2.2 | 113466_Lymph Node Pool | 1.7 |
| effusion)_MDA-MB-231 | | | |
| 112189_ductal cell | 3.0 | 103372_Fetal Skeletal Muscle | 0.7 |
| carcinoma (breast) | | | |
| Breast ca. (pleural effusion)_T47D | 18.5 | 113456_Skeletal Muscle Pool | 1.1 |
| Breast carcinoma_MDA-N | 0.7 | 113459_Spleen Pool | 2.5 |
| 113452_Breast Pool | 1.5 | 113462_Thymus Pool | 2.4 |
| 103398_Trachea | 1.2 | CNS ca. (glio/astro)_U87-MG | 2.7 |
| 112354_lung | 0.4 | CNS ca. (glio/astro)_U-118-MG | 3.0 |
| 103374_Fetal Lung | 2.3 | CNS ca. (neuro; met)_SK-N-AS | 2.1 |
| 94921_Small cell carcinoma of the | 0.2 | 95264_Brain astrocytoma | 0.6 |
| lung | | | |
| Lung ca. (small cell)_LX-1 | 3.3 | CNS ca. (astro)_SNB-75 | 1.8 |
| 94919_Small cell carcinoma of the | 0.5 | CNS ca. (glio)_SNB-19 | 4.1 |
| lung | | | |
| Lung ca. (s.cell var.)_SHP-77 | 2.4 | CNS ca. (glio)_SF-295 | 2.1 |
| 95268_Lung (Large cell | 0.6 | 113447_Brain (Amygdala) Pool | 0.9 |
| carcinoma) | | | |
| 94920_Small cell carcinoma of the | 0.6 | 103382_Brain (cerebellum) | 1.9 |
| lung | | | |
| Lung ca. (non-s.cell)_NCI-H23 | 3.7 | 64019-1_brain (fetal) | 2.8 |
| Lung ca. (large cell)_NCI-H460 | 0.9 | 113448_Brain (Hippocampus) | 1.0 |
| | | Pool | |
| Lung ca. (non-s.cell)_HOP-62 | 1.2 | 113464_Cerebral Cortex Pool | 0.7 |
| Lung ca. (non-s.cl)_NCI-H522 | 1.7 | 113449_Brain (Substantia nigra) | 0.9 |
| | | Pool | |
| 103392_Liver | 26.6 | 113450_Brain (Thalamus) Pool | 1.0 |
| 103393_Fetal Liver | 45.5 | 103384_Brain (whole) | 1.6 |
| Liver ca. (hepatoblast)_HepG2 | 100.0 | 113458_Spinal Cord Pool | 1.7 |
| 113465_Kidney Pool | 1.7 | 103375_Adrenal Gland | 3.1 |
| 103373_Fetal Kidney | 11.1 | 113454_Pituitary gland Pool | 1.7 |
| Renal ca._786-0 | 1.0 | 103397_Salivary Gland | 1.0 |
| 112188_renal cell carcinoma | 0.3 | 103369_Thyroid (female) | 2.8 |
| Renal ca._ACHN | 1.4 | Pancreatic ca._CAPAN2 | 0.8 |
| 112190_Renal cell carcinoma | 1.8 | 113453_Pancreas Pool | 7.5 |

TABLE 70
Panel 2.2

| | Relative | | Relative |
|---|---|---|---|
| | Expression (%) | | Expression (%) |
| | 2.2x4tm6408f_{—} | | 2.2x4tm6408f_{—} |
| Tissue Name | ag3086_a1 | Tissue Name | ag3086_a1 |
| Normal Colon GENPAK 061003 | 1.4 | 83793 Kidney NAT (OD04348) | 40.9 |
| 97759 Colon cancer (OD06064) | 0.1 | 98938 Kidney malignant cancer | 0.4 |
| | | (OD06204B) | |
| 97760 Colon cancer NAT | 0.0 | 98939 Kidney normal adjacent | 5.1 |
| (OD06064) | | tissue (OD06204E) | |
| 97778 Colon cancer (OD06159) | 0.0 | 85973 Kidney Cancer (OD04450- | 5.7 |
| | | 01) | |
| 97779 Colon cancer NAT | 1.4 | 85974 Kidney NAT (OD04450-03) | 15.0 |
| (OD06159) | | | |
| 98861 Colon cancer (OD06297-04) | 0.0 | Kidney Cancer Clontech 8120613 | 0.2 |
| 98862 Colon cancer NAT | 1.1 | Kidney NAT Clontech 8120614 | 5.9 |
| (OD06297-015) | | | |
| 83237 CC Gr.2 ascend colon | 0.4 | Kidney Cancer Clontech 9010320 | 0.4 |
| (ODO3921) | | | |
| 83238 CC NAT (ODO3921) | 0.2 | Kidney NAT Clontech 9010321 | 1.5 |
| 97766 Colon cancer metastasis | 0.0 | Kidney Cancer Clontech 8120607 | 0.1 |
| (OD06104) | | | |
| 97767 Lung NAT (OD06104) | 0.6 | Kidney NAT Clontech 8120608 | 3.5 |
| 87472 Colon mets to lung | 1.3 | Normal Uterus GENPAK 061018 | 0.2 |
| (OD04451-01) | | | |
| 87473 Lung NAT (OD04451-02) | 0.4 | Uterus Cancer GENPAK 064011 | 0.1 |
| Normal Prostate Clontech A+ | 0.6 | Normal Thyroid Clontech A+ | 0.5 |
| 6546-1 (8090438) | | 6570-1 (7080817) | |
| 84140 Prostate Cancer (OD04410) | 0.2 | Thyroid Cancer GENPAK 064010 | 0.3 |
| 84141 Prostate NAT (OD04410) | 0.5 | Thyroid Cancer INVITROGEN | 2.1 |
| | | A302152 | |
| Normal Ovary Res. Gen. | 0.4 | Thyroid NAT INVITROGEN | 0.4 |
| | | A302153 | |
| 98863 Ovarian cancer (OD06283- | 0.2 | Normal Breat GENPAK 061019 | 0.7 |
| 03) | | | |
| 98865 Ovarian cancer | 1.2 | 84877 Breast Cancer (OD04566) | 2.3 |
| NAT/fallopian tube (OD06283-07) | | | |
| Ovarian Cancer GENPAK 064008 | 1.5 | Breast Cancer Res. Gen. 1024 | 1.9 |
| 97773 Ovarian cancer (OD06145) | 0.9 | 85975 Breast Cancer (OD04590- | 5.1 |
| | | 01) | |
| 97775 Ovarian cancer NAT | 1.8 | 85976 Breast Cancer Mets | 1.3 |
| (OD06145) | | (OD04590-03) | |
| 98853 Ovarian cancer (OD06455- | 0.6 | 87070 Breast Cancer Metastasis | 0.7 |
| 03) | | (OD04655-05) | |
| 98854 Ovarian NAT (OD06455- | 0.2 | GENPAK Breast Cancer 064006 | 0.5 |
| 07) Fallopian tube | | | |
| Normal Lung GENPAK 061010 | 0.8 | Breast Cancer Clontech 9100266 | 0.2 |
| 92337 Invasive poor diff. lung | 0.3 | Breast NAT Clontech 9100265 | 0.5 |
| adeno (ODO4945-01 | | | |
| 92338 Lung NAT (ODO4945-03) | 1.1 | Breast Cancer INVITROGEN | 0.2 |
| | | A209073 | |
| 84136 Lung Malignant Cancer | 1.6 | Breast NAT INVITROGEN | 1.4 |
| (OD03126) | | A2090734 | |
| 84137 Lung NAT (OD03126) | 0.3 | 97763 Breast cancer (OD06083) | 0.7 |
| 90372 Lung Cancer (OD05014A) | 0.5 | 97764 Breast cancer node | 0.2 |
| | | metastasis (OD06083) | |
| 90373 Lung NAT (OD05014B) | 0.6 | Normal Liver GENPAK 061009 | 28.7 |
| 97761 Lung cancer (OD06081) | 0.5 | Liver Cancer Research Genetics | 7.5 |
| | | RNA 1026 | |
| 97762 Lung cancer NAT | 1.2 | Liver Cancer Research Genetics | 45.0 |
| (OD06081) | | RNA 1025 | |
| 85950 Lung Cancer (OD04237-01) | 0.3 | Paired Liver Cancer Tissue | 35.7 |
| | | Research Genetics RNA 6004-T | |
| 85970 Lung NAT (OD04237-02) | 1.1 | Paired Liver Tissue Research | 5.1 |
| | | Genetics RNA 6004-N | |
| 83255 Ocular Mel Met to Liver | 0.2 | Paired Liver Cancer Tissue | 14.7 |
| (ODO4310) | | Research Genetics RNA 6005-T | |
| 83256 Liver NAT (ODO4310) | 21.6 | Paired Liver Tissue Research | 65.0 |
| | | Genetics RNA 6005-N | |
| 84139 Melanoma Mets to Lung | 0.2 | Liver Cancer GENPAK 064003 | 100.0 |
| (OD04321) | | | |
| 84138 Lung NAT (OD04321) | 0.2 | Normal Bladder GENPAK 061001 | 2.8 |
| Normal Kidney GENPAK 061008 | 5.5 | Bladder Cancer Research Genetics | 0.2 |
| | | RNA 1023 | |
| 83786 Kidney Ca, Nuclear grade 2 | 20.6 | Bladder Cancer INVITROGEN | 0.7 |
| (OD04338) | | A302173 | |
| 83787 Kidney NAT (OD04338) | 4.5 | Normal Stomach GENPAK | 2.5 |
| | | 061017 | |
| 83788 Kidney Ca Nuclear grade | 6.5 | Gastric Cancer Clontech 9060397 | 0.0 |
| 1/2 (OD04339) | | | |
| 83789 Kidney NAT (OD04339) | 6.0 | NAT Stomach Clontech 9060396 | 0.1 |
| 83790 Kidney Ca, Clear cell type | 0.4 | Gastric Cancer Clontech 9060395 | 0.0 |
| (OD04340) | | | |
| 83791 Kidney NAT (OD04340) | 8.7 | NAT Stomach Clontech 9060394 | 0.3 |
| 83792 Kidney Ca, Nuclear grade 3 | 0.3 | Gastric Cancer GENPAK 064005 | 2.8 |
| (OD04348) | | | |

TABLE 71
Panel 4D

| | Relative | | Relative |
|---|---|---|---|
| | Expression (%) | | Expression (%) |
| | 4dx4tm5510f_{—} | | 4dx4tm5510f_{—} |
| Tissue Name | ag3086_a2 | Tissue Name | ag3086_a2 |
| 93768_Secondary Th1_anti- | 0.7 | 93100_HUVEC (Endothelial)_IL- | 0.4 |
| CD28/anti-CD3 | | lb | |
| 93769_Secondary Th2_anti- | 0.9 | 93779_HUVEC (Endothelial)_IFN | 1.2 |
| CD28/anti-CD3 | | gamma | |
| 93770_Secondary Tr1_anti- | 1.1 | 93102_HUVEC | 0.3 |
| CD28/anti-CD3 | | (Endothelial)_TNF alpha + IFN | |
| | | gamma | |
| 93573_Secondary Th1_resting day | 2.8 | 93101_HUVEC | 0.2 |
| 4-6 in IL-2 | | (Endothelial)_TNF alpha + IL4 | |
| 93572_Secondary Th2_resting day | 1.4 | 93781_HUVEC (Endothelial)_IL- | 0.8 |
| 4-6 in IL-2 | | 11 | |
| 93571_Secondary Tr1_resting day | 1.3 | 93583_Lung Microvascular | 1.0 |
| 4-6 in IL-2 | | Endothelial Cells_none | |
| 93568_primary Th1_anti- | 0.7 | 93584_Lung Microvascular | 0.9 |
| CD28/anti-CD3 | | Endothelial Cells_TNFa (4 ng/ml) | |
| | | and IL1b (1 ng/ml) | |
| 93569_primary Th2_anti- | 0.9 | 92662_Microvascular Dermal | 1.6 |
| CD28/anti-CD3 | | endothelium_none | |
| 93570_primary Tr1_anti- | 1.1 | 92663_Microvascular Dermal | 0.8 |
| CD28/anti-CD3 | | endothelium_TNFa (4 ng/ml) and | |
| | | IL1b (1 ng/ml) | |
| 93565_primary Th1_resting dy 4-6 | 6.4 | 93773_Bronchial | 3.2 |
| in IL-2 | | epithelium_TNFa (4 ng/ml) and | |
| | | IL1b (1 ng/ml)** | |
| 93566_primary Th2_resting dy 4-6 | 3.1 | 93347_Small Airway | 1.8 |
| in IL-2 | | Epithelium_none | |
| 93567_primary Tr1_resting dy 4-6 | 2.0 | 93348_Small Airway | 3.3 |
| in IL-2 | | Epithelium_TNFa (4 ng/ml) and | |
| | | IL1b (1 ng/ml) | |
| 93351_CD45RA CD4 | 0.4 | 92668_Coronery Artery | 1.0 |
| lymphocyte_anti-CD28/anti-CD3 | | SMC_resting | |
| 93352 CD45RO CD4 | 1.4 | 92669 Coronery Artery | 0.6 |
| lymphocyte_anti-CD28/anti-CD3 | | SMC_TNFa (4 ng/ml) and IL1b (1 | |
| | | ng/ml) | |
| 93251_CD8 Lymphocytes_anti- | 1.4 | 93107_astrocytes_resting | 3.2 |
| CD28/anti-CD3 | | | |
| 93353_chronic CD8 Lymphocytes | 1.7 | 93108_astrocytes_TNFa (4 ng/ml) | 2.7 |
| 2ry_resting dy 4-6 in IL-2 | | and IL1b (1 ng/ml) | |
| 93574_chronic CD8 Lymphocytes | 0.6 | 92666_KU-812 (Basophil)_resting | 1.5 |
| 2ry_activated CD3/CD28 | | | |
| 93354_CD4_none | 2.1 | 92667_KU-812 | 1.2 |
| | | (Basophil)_PMA/ionoycin | |
| 93252_Secondary | 4.2 | 93579_CCD1106 | 1.0 |
| Th1/Th2/Tr1_anti-CD95 CH11 | | (Keratinocytes)_none | |
| 93103_LAK cells_resting | 1.2 | 93580_CCD1106 | 3.6 |
| | | (Keratinocytes)_TNFa and | |
| | | IFNg** | |
| 93788_LAK cells_IL-2 | 3.7 | 93791_Liver Cirrhosis | 84.6 |
| 93787_LAK cells_IL-2 + IL-12 | 2.2 | 93792_Lupus Kidney | 33.9 |
| 93789_LAK cells_IL-2 + IFN | 3.0 | 93577_NCI-H292 | 8.6 |
| gamma | | | |
| 93790_LAK cells_IL-2 + IL-18 | 2.6 | 93358_NCI-H292_IL-4 | 7.1 |
| 93104_LAK | 1.0 | 93360_NCI-H292_IL-9 | 6.5 |
| cells_PMA/ionomycin and IL-18 | | | |
| 93578_NK Cells IL-2_resting | 1.5 | 93359_NCI-H292_IL-13 | 2.8 |
| 93109_Mixed Lymphocyte | 2.2 | 93357_NCI-H292_IFN gamma | 3.1 |
| Reaction_Two Way MLR | | | |
| 93110_Mixed Lymphocyte | 1.2 | 93777_HPAEC_- | 1.7 |
| Reaction_Two Way MLR | | | |
| 93111_Mixed Lymphocyte | 1.3 | 93778_HPAEC_IL-1 beta/TNA | 1.4 |
| Reaction_Two Way MLR | | alpha | |
| 93112_Mononuclear Cells | 0.7 | 93254_Normal Human Lung | 4.3 |
| (PBMCs)_resting | | Fibroblast_none | |
| 93113_Mononuclear Cells | 0.8 | 93253_Normal Human Lung | 4.9 |
| (PBMCs)_PWM | | Fibroblast_TNFa (4 ng/ml) and IL- | |
| | | 1b (1 ng/ml) | |
| 93114_Mononuclear Cells | 0.6 | 93257_Normal Human Lung | 2.2 |
| (PBMCs)_PHA-L | | Fibroblast_IL-4 | |
| 93249_Ramos (B cell)_none | 1.9 | 93256_Normal Human Lung | 1.2 |
| | | Fibroblast_IL-9 | |
| 93250_Ramos (B cell)_ionomycin | 1.4 | 93255_Normal Human Lung | 1.6 |
| | | Fibroblast_IL-13 | |
| 93349_B lymphocytes_PWM | 1.2 | 93258_Normal Human Lung | 1.9 |
| | | Fibroblast_IFN gamma | |
| 93350_B lymphoytes_CD40L and | 2.2 | 93106_Dermal Fibroblasts | 3.3 |
| IL-4 | | CCD1070_resting | |
| 92665_EOL-1 | 2.4 | 93361_Dermal Fibroblasts | 4.7 |
| (Eosinophil)_dbcAMP | | CCD1070_TNF alpha 4 ng/ml | |
| differentiated | | | |
| 93248_EOL-1 | 2.6 | 93105_Dermal Fibroblasts | 0.7 |
| (Eosinophil)_dbcAMP/ | | CCD1070_IL-1 beta 1 ng/ml | |
| PMAionomycin | | | |
| 93356_Dendritic Cells_none | 2.2 | 93772_dermal fibroblast_IFN | 0.8 |
| | | gamma | |
| 93355_Dendritic Cells_LPS 100 | 2.1 | 93771_dermal fibroblast_IL-4 | 2.4 |
| ng/ml | | | |
| 93775_Dendritic Cells_anti-CD40 | 1.8 | 93260_IBD Colitis 2 | 11.6 |
| 93774_Monocytes_resting | 1.5 | 93261_IBD Crohns | 14.2 |
| 93776_Monocytes_LPS 50 ng/ml | 0.6 | 735010_Colon_normal | 61.0 |
| 93581_Macrophages_resting | 1.7 | 735019_Lung_none | 3.6 |
| 93582_Macrophages_LPS 100 | 1.1 | 64028-1_Thymus_none | 100.0 |
| ng/ml | | | |
| 93098_HUVEC | 1.3 | 64030-1_Kidney_none | 5.7 |
| (Endothelial)_none | | | |
| 93099_HUVEC | 1.2 | | |
| (Endothelial)_starved | | | |

TABLE 72
Panel 4.1D

| | Relative Expression (%) | |
|---|---|---|
| Tissue Name | 4.1dx4tm5986f_ag3797_a1 | 4.1dtm6034f_ag3797 |
| 93768_Secondary Th1_anti-CD28/anti-CD3 | 8.9 | 1.3 |
| 93769_Secondary Th2_anti-CD28/anti-CD3 | 2.6 | 1.3 |
| 93770_Secondary Tr1_anti-Cd28/anti-CD3 | 3.1 | 0.9 |
| 93573_Secondary Th1_resting day 4-6 in IL-2 | 1.5 | 1.6 |
| 93572_Secondary Th2_resting day 4-6 in IL-2 | 3.4 | 0.7 |
| 93571_Secondary Tr1_resting day 4-6 in IL-2 | 3.4 | 0.9 |
| 93568_primary TH1_anti-CD28/anti-CD3 | 3.2 | 0.3 |
| 93569_primary Th2_anti-CD28/anti-CD3 | 2.0 | 1.1 |
| 93570_primary Tr1_anti-CD28/anti-CD3 | 2.7 | 1.0 |
| 93565_primary Th1_resting dy 4-6 in IL-2 | 3.2 | 0.4 |
| 93566_primary Th2_resting dy 4-6 in IL-2 | 4.5 | 0.0 |
| 93567_primary Tr1_resting dy 4-6 in IL-2 | 2.3 | 0.7 |
| 93351_CD45RA CD4 lymphocyte_anti-CD28/anti-CD3 | 2.0 | 0.7 |
| 93352_CD45RO CD4 lymphocyte_anti-CD28/anti-CD3 | 1.8 | 2.0 |
| 93251_CD8 Lymphocyte_anti-CD28/anti-CD3 | 5.6 | 0.9 |
| 93353_chronic CD8 Lymphocytes 2ry_resting dy 4-6 in IL-2 | 4.6 | 1.1 |
| 93574_chronic CD8 Lymphocytes 2ry_activated CD3/CD28 | 1.8 | 0.2 |
| 93354_CD4_none | 7.2 | 1.3 |
| 93252_Secondary Th1/Th2/Tr1_anti-CD95 CH11 | 6.6 | 1.1 |
| 93103_LAK cells_resting | 7.6 | 0.4 |
| 93788_LAK cells_IL-2 | 4.8 | 0.3 |
| 93787_LAK cells_IL-2 + IL-12 | 5.7 | 0.8 |
| 93789_LAK cells_IL-2 + IFN gamma | 5.5 | 0.2 |
| 93790_LAK cells_IL-2 + IL-18 | 1.6 | 0.4 |
| 93104_LAK cells_PMA/ionomycin and IL-18 | 2.7 | 1.3 |
| 93578_NK Cells IL-2_resting | 5.6 | 2.0 |
| 93109_Miced Lymphocyte Reaction_Two Way MLR | 4.9 | 2.0 |
| 93110_Mixed Lymphocyte Reaction_Two Way MLR | 0.5 | 0.8 |
| 93111_Mixed Lymphocyte Reaction_Two Way MLR | 6.0 | 0.2 |
| 93112_Mononuclear Cells (PBMCs)_resting | 1.3 | 0.4 |
| 93113_Mononuclear Cells (PBMCs)_PWM | 7.9 | 0.5 |
| 93114_Mononuclear Cells (PBMCs)_PHA-L | 5.1 | 0.5 |
| 93249_Ramos (B cell)_none | 5.7 | 0.6 |
| 93250_Ramos (B cell)_ionomycin | 4.4 | 0.3 |
| 93349_B lymphocytes_PWM | 1.1 | 0.2 |
| 93350_B lymphocytes_CD40L and IL-4 | 4.3 | 0.6 |
| 92665_EOL-1 (Eosinophil)_dbcAMP differentiated | 8.4 | 3.5 |
| 93248_EOL-1 (Eosinophil)_dbcAMP/PMAionomycin | 7.2 | 5.1 |
| 93356_Dendritic Cells_none | 3.4 | 1.0 |
| 93355_Dendritic Cells_LPS 100 ng/ml | 5.5 | 0.5 |
| 93775_Dendritic Cells_anti-CD40 | 2.6 | 0.3 |
| 93774_Monocytes_resting | 1.1 | 0.9 |
| 93776_Monocytes_LPS 50 ng/ml | 2.6 | 0.3 |
| 93581_Macrophages_resting | 5.2 | 0.2 |
| 93582_Macrophages_LPS 100 ng/ml | 1.4 | 0.3 |
| 93098_HUVEC (Endothelial)_none | 1.2 | 0.2 |
| 93099_HUVEC (Endothelial)_starved | 3.4 | 0.2 |
| 93100_HUVEC (Endothelial)_IL-1b | 2.4 | 0.1 |
| 93779_HUVEC (Endothelial)_IFN gamma | 2.6 | 1.2 |
| 93102_HUVEC (Endothelial)_TNF alpha + IFN alpha | 0.0 | 0.3 |
| 93101_HUVEC (Endothelial)_TNF alpha + IL4 | 1.6 | 0.4 |
| 93781_HUVEC (Endothelial)_IL-11 | 2.2 | 0.4 |
| 93583_Lung Microvascular Endothelial Cells_none | 1.8 | 0.9 |
| 93584_Lung Microvascular Endothelial Cells_TNFa (4 ng/ml) and | 2.2 | 0.3 |
| IL1b (1 ng/ml) | | |
| 92662_Microvascular Dermal endothelium_none | 1.4 | 0.5 |
| 92663_Microvascular Dermal endothelium_TNFa (4 ng/ml) and IL1b | 2.2 | 0.3 |
| (1 ng/ml) | | |
| 93773_Bronchial epithelium_TNFa (4 ng/ml) and IL1b (1 ng/ml)** | 17.8 | 0.5 |
| 93347_Small Airway Epithelium_none | 1.5 | 0.2 |
| 93348_Small Airway Epithelium_TNFa (4 ng/ml) and IL1b (1 ng/ml) | 3.0 | 0.6 |
| 92668_Coronery Artery SMC_resting | 1.1 | 0.6 |
| 92669_Coronery Artery SMC_TNFa (4 ng/ml) and IL1b (1 ng/ml) | 1.9 | 0.8 |
| 93107_astrocytes_resting | 2.5 | 1.5 |
| 93108_astrocytes_TNFa (4 ng/ml) and IL1b (1 ng/ml) | 0.5 | 1.2 |
| 92666_KU-182 (Basophil)_resting | 4.3 | 0.8 |
| 92667_KU-182 (Basophil)_PMA/ionomycin | 3.0 | 0.6 |
| 93579_CCD1106 (Keratinocytes)_none | 1.6 | 0.9 |
| 93580_CCD1106 (Keratinocytes)_TNFa and IFNg** | 2.4 | 0.0 |
| 93791_Liver Cirrhosis | 76.6 | 9.8 |
| 93577_NCI-H292 | 5.4 | 4.1 |
| 93358_NCI-H292_IL-4 | 10.3 | 0.6 |
| 93360_NCI-H292_IL-9 | 16.5 | 1.3 |
| 93359_NCI-H292_IL-13 | 8.5 | 3.6 |
| 93357_NCI-H292_IFN-gamma | 5.8 | 3.4 |
| 93777_HPAEC_- | 1.2 | 1.0 |
| 93778_HPAEC_IL-1 beta/TNA alpha | 1.5 | 0.3 |
| 93254_Normal Human Lung Fibroblast_none | 2.5 | 0.8 |
| 93253_Normal Human Fibroblast_TNFa (4 ng/ml) and IL-1b (1 | 5.7 | 0.9 |
| ng/ml) | | |
| 93257_Normal Human Lung Fibroblast_IL-4 | 2.9 | 0.4 |
| 93256_Normal Human Lung Fibroblast_IL-9 | 2.5 | 0.4 |
| 93255_Notmal Human Lung Fibroblast_IL-13 | 2.7 | 1.9 |
| 93258_Normal Human Lung Fibroblast_IFN gamma | 0.0 | 2.2 |
| 93106_Dermal Fibroblasts CCD1070_resting | 6.1 | 3.4 |
| 93361_Dermal Fibroblasts CCD1070_TNF alpha 4 ng/ml | 2.1 | 3.8 |
| 93105_Dermal Fibroblasts CCD1070_IL-1 beta 1 ng/ml | 4.0 | 1.5 |
| 93772_dermal fibroblast_IFN gamma | 1.6 | 0.9 |
| 93771_dermal fibroblast_IL-4 | 1.4 | 1.3 |
| 93892_Dermal fibroblasts_none | 2.3 | 1.5 |
| 99202_Neutrophils_TNFa + LPS | 0.0 | 1.7 |
| 99203_Neutrophils_none | 0.8 | 0.6 |
| 735010_Colon_nornmal | 21.7 | 6.7 |
| 735019_Lung_none | 3.7 | 10.6 |
| 64028-1_Thymus_none | 11.7 | 27.0 |
| 64030-1_Kidney_none | 100.0 | 100.0 |

TABLE 73
Panel CNS_Neurodegeneration_v1.0

| | Relative |
|---|---|
| | Expression (%) |
| Tissue Name | tm7142f_ag3797_{‘3}b2 |
| AD 1 Hippo | 53.6 |
| AD 2 Hippo | 69.7 |
| AD 3 Hippo | 25.6 |
| AD 4 Hiipo | 33.9 |
| AD 5 Hippo | 91.6 |
| AD 6 Hippo | 39.7 |
| Control 2 Hippo | 38.4 |
| Control 4 Hippo | 59.4 |
| Control (Path) 3 Hippo | 7.8 |
| AD 1 Temporal Ctx | 41.0 |
| AD 2 Temporal Ctx | 70.4 |
| AD 3 Temporal Ctx | 21.3 |
| AD 4 Temporal Ctx | 46.7 |
| AD 5 Inf Temporal Ctx | 92.1 |
| AD 5 Sup Temporal Ctx | 74.8 |
| AD 6 Inf Temporal Ctx | 44.5 |
| AD 6 Sup Temporal Ctx | 57.9 |
| Control 1 Temporal Ctx | 22.8 |
| Control 2 Temporal Ctx | 45.7 |
| Control 3 Temporal Ctx | 13.8 |
| Control 3 Temporal Ctx | 51.4 |
| Control (Path) 1 Temporal Ctx | 62.8 |
| Control (Path) 2 Temporal Ctx | 41.4 |
| Control (Path) 3 Temporal Ctx | 12.5 |
| Control (Path) 4 Temporal Ctx | 62.2 |
| AD 1 Occipital Ctx | 48.0 |
| AD 2 Occipital Ctx (Missing) | 0.0 |
| AD 3 Occipital Ctx | 13.0 |
| AD 4 Occipital Ctx | 40.7 |
| AD 5 Occipital Ctx | 51.9 |
| AD 6 Occipital Ctx | 28.4 |
| Control 1 Occipital Ctx | 2.6 |
| Control 2 Occipital Ctx | 100.0 |
| Control 3 Occipital Ctx | 33.1 |
| Control 4 Occipital Ctx | 18.6 |
| Control (Path) 1 Occipital Ctx | 82.7 |
| Control (Path) 2 Occipital Ctx | 18.7 |
| Control (Path) 3 Occipital Ctx | 3.4 |
| Control (Path) 4 Occipital Ctx | 49.0 |
| Control 1 Parietal | 19.8 |
| Control 2 Pareital | 60.6 |
| Control 3 Parietal | 31.0 |
| Control (Path) 1 Parietal | 57.3 |
| Control (Path) 2 Parietal | 31.0 |
| Control (Path) 3 Parietal | 5.7 |
| Control (Path) 4 Parietal | 52.1 |

Panel 1.3D Summary Ag3086 The NOV11a gene is highly expressed in both fetal and adult liver tissue (CTs=26) and liver cancer cell lines (CT=27). The gene is also expressed at moderate to low levels in most of the other tissues in the panel. Thus, since the NOV11a gene appears to be highly expressed in liver tissue, it could therefore be used to distinguish liver derived tissue from other tissues. The NOV11a gene product may also be a potential therapeutic treatment of disease in any of these tissues.

In tissues involved in the central nervous system, the NOV11a gene is moderately expressed in the fetal and adult brain, including the adult thalamus, substantia nigra, hippocampus, amygdala and is also expressed at low but significant levels in the cerebellum and cerebral cortex. This expression profile suggests that the NOV11a gene has functional significance in the CNS. The close homologue to the NOV11a gene product, hepatocyte growth factor, has numerous therapeutic applications in the CNS, including prevention of neuronal death in animal models of stroke and ischemia. Hepatocyte growth factor has mitogenic activity, crossing the blood brain barrier when disrupted, and thus has potential application as a protein therapeutic to treat brain pathologies when administered directly to the cortico spinal fluid or systemically when the blood brain barrier is disrupted. Hepatocyte growth factor-like protein is a neurotrophic factor useful in the prevention of motoneuron atrophy upon axotomy. Therefore, the protein encoded by the NOV11a gene may be useful as a therapeutic agent in treating stroke and neurodegenerative diseases including Alzheimer's disease, Parkinson's disease, and Huntington's disease. The potential role of the NOV11a gene or its protein product in brain plasticity and regeneration affords utility in treating brain damage and aging related disorders, such as memory impairment that has hippocampal dysfunction as its primary focus.

General_Screening_Panel_{—}1.4 Ag3797 The expression of the NOV11a gene in panel 1.4 appears to be highest in a sample derived from a liver cancer cell line (HepG2) (CT=25.3). In addition there is substantial expression of this gene associated with other liver derived material (adult liver CT=27.2; fetal liver CT=26.5). Thus, the expression of the NOV11a gene could be used to distinguish liver derived specimens from other samples. In addition, therapeutic modulation of this gene might be of benefit in the treatment of liver related disorders.

Panel 2.2 Summary Ag3086 The expression of the NOV11a gene appears to be highest in a sample derived from a liver cancer specimen (CT=26) and is also significant in a number of samples derived from liver tissue. This result is consistent with what is seen in Panels 1.4 and 2D. In addition there appears to be substantial expression of this gene associated with normal kidney tissue (CT=27.2) when compared to adjacent kidney cancer specimens. Thus, this gene could be used to distinguish liver tissue from non-liver tissue as well as distinguish normal kidney tissue when compared to adjacent kidney cancer. Moreover, therapeutic modulation of the expression of the NOV11a gene or function of its product might be of benefit in the treatment of kidney cancer.

Panel 4D Summary Ag3086 The NOV11a gene is highly expressed in the thymus (CT=24), colon (CT=28.4), and IBD Colitis 2 (CT=27.2) and is expressed at lower levels in mature T cells. The NOV11a gene encodes a putative hepatocyte like growth factor homologue. There are reports that hepatocyte growth factor (HGF) is expressed in the thymus and colon. In the thymus, HGF may promote T cell production and in the colon, overexpression of HGF has been shown to leads to IBD like disease in mice. Therapies designed with the protein encoded for by the NOV11a gene could be important in the regulation of T cell development and immune function and be useful in organ transplantation. In addition, blocking the function of the NOV11a gene product could help in the treatment of IBD colitis.

Panel 4.1D Summary Ag3797 Results from two experiments using the same probe and primer set are in very good agreement. In both experiments, highest expression of the NOV11a gene is detected in kidney (CT=29, 27.4). Moderate expression is also detected in liver cirrhosis (CT=29.4, 30.7). Moderate to low expression of the gene is detected in many of the tissues in this panel. Thus, expression of the NOV11a gene could be used to distinguish those tissues from other tissues.

Panel CNS_Neurodegeneration_v1.0 Summary Ag3797 Highest expression of the NOV11a gene is detected in the occipital cortex of a control patient (CT=3 1.3). Moderate to low expression is detected throughought the tissue samples in this panel. Please see panel 1.3 for a discussion of potential utility of this gene with regards to the CNS. (Korhonen et al., Eur J Neurosci. 12:3453-61, 2000; Powell et al., Neuron 30:79-89, 2001; Stella et al., Mol Biol Cell 12:1341-52, 2001; Kern et al., Cytokine 14:170-6, 2001; Hayashi et al., Gene Ther 8:1167-73, 2001; Tamura et al., Scand J Immunol. 47:296-301, 1998; Takayama et al., Lab Invest. 81:297-305, 2001.)

NOV12: 26S Protease Regulatory Subunit-like

Expression of the NOV12 gene (GMAC023940_A) was assessed using the primer-probe set Ag1505 described in Table 74. Results from RTQ-PCR runs are shown in Tables 75, 76, 77, and 78.

TABLE 74

Probe Name Ag1505

| | | | | | |
|---|---|---|---|---|---|
| | | | | Start | |
| Primers | Sequences | TM | Length | Position | SEQ ID NO: |

| | | | | | |
|---|---|---|---|---|---|
| Forward | 5′-GAAGAAGCCCATCTTTCAGATT-3′ | 58.8 | 22 | 1140 | 194 |
| Probe | TET-5′- | 68.7 | 26 | 1188 | 195 |
| | TGATGTAACCCTGCACGACTTGATCA-3′ | | | | |
| | TAMRA | | | | |
| Reverse | 5′-AGCACCAGAGAGGTCATCTTTA-3′ | 58.1 | 22 | 1218 | 196 |

TABLE 75
Panel 1.2

| | Relative | | Relative |
|---|---|---|---|
| | Expression (%) | | Expression (%) |
| | 1.2tm2128t_{—} | | 1.2tm2128t_{—} |
| Tissue Name | ag1505 | Tissue Name | ag1505 |
| Endothelial cells | 0.5 | Renal ca. 786-0 | 0.0 |
| Heart (fetal) | 0.3 | Renal ca. A498 | 0.0 |
| Pancreas | 0.0 | Renal ca. RXF 393 | 0.0 |
| Pancreatic ca. CAPAN 2 | 0.0 | Renal ca. ACHN | 0.0 |
| Adrenal Gland (new lot*) | 0.0 | Renal ca. UO-31 | 0.0 |
| Thyroid | 0.0 | Renal ca. TK-10 | 0.0 |
| Salavary gland | 26.6 | Liver | 24.1 |
| Pituitary gland | 0.0 | Liver (fetal) | 19.6 |
| Brain (fetal) | 3.4 | Liver ca. (hepatoblast) HepG2 | 0.0 |
| Brain (whole) | 0.0 | Lung | 0.0 |
| Brain (amygdala) | 2.6 | Lung (fetal) | 0.0 |
| Brain (cerebellum) | 2.8 | Lung ca. (small cell) LX-1 | 0.0 |
| Brain (hippocampus) | 13.2 | Lung ca. (small cell) NCI-H69 | 0.4 |
| Brain (thalamus) | 0.2 | Lung ca. (s.cell var.) SHP-77 | 0.0 |
| Cerebral Cortex | 0.6 | Lung ca. (large cell) NCI-H460 | 0.0 |
| Spinal cord | 0.0 | Lung ca. (non-sm. cell) A549 | 1.1 |
| CNS ca. (glio/astro) U87-MG | 0.0 | Lung ca. (non-s.cell) NCI-H23 | 0.0 |
| CNS ca. (glio/astro) U-118-MG | 0.3 | Lung ca (non-s.cell) HOP-62 | 0.0 |
| CNS ca. (astro) SW1783 | 0.0 | Lung ca. (non-s.cl) NCI-H522 | 0.0 |
| CNS ca.* (neuro; met) SK-N-AS | 5.8 | Lung ca. (squam.) SW 900 | 0.0 |
| CNS ca. (astro) SF-539 | 0.0 | Lung ca. (squam.) NCI-H596 | 0.0 |
| CNS ca. (astro) SNB-75 | 0.0 | Mammary gland | 21.0 |
| CNS ca. (glio) SNB-19 | 0.6 | Breast ca.* (pl. effusion) MCF-7 | 0.0 |
| CNS ca. (glio) U251 | 14.1 | Breast ca.* (pl.ef) MDA-MB-231 | 1.8 |
| CNS ca. (glio) SF-295 | 0.0 | Breast ca.* (pl. effusion) T47D | 0.2 |
| Heart | 0.2 | Breast ca. BT-549 | 0.0 |
| Skeletal Muscle (new lot*) | 0.2 | Breast ca. MDA-N | 0.0 |
| Bone marrow | 0.5 | Ovary | 0.0 |
| Thymus | 0.0 | Ovarian ca. OVCAR-3 | 0.0 |
| Spleen | 0.0 | Ovarian ca. OVCAR-4 | 0.0 |
| Lymph node | 0.0 | Ovarian ca. OVCAR-5 | 0.0 |
| Colorectal | 2.0 | Ovarian ca. OVCAR-8 | 0.0 |
| Stomach | 11.7 | Ovarian ca. IGROV-1 | 0.0 |
| Small intestine | 0.3 | Ovarian ca.* (ascites) SK-OV-3 | 0.0 |
| Colon ca. SW480 | 0.0 | Uterus | 0.0 |
| Colon ca.* (SW480 met) SW620 | 0.0 | Placenta | 0.0 |
| Colon ca. HT29 | 0.0 | Prostate | 0.0 |
| Colon ca. HCT-116 | 0.0 | Prostate ca.* (bone met) PC-3 | 0.0 |
| Colon ca. CaCo-2 | 0.0 | Testis | 0.5 |
| 83219 CC Well to Mod Diff | 1.5 | Melanoma Hs688(A).T | 0.0 |
| (ODO3866) | | | |
| Colon ca. HCC-2998 | 0.0 | Melanoma* (met) Hs688(B).T | 0.2 |
| Gastric ca.* (liver met) NCI-N87 | 100.0 | Melanoma UACC-62 | 0.0 |
| Bladder | 45.4 | Melanoma M14 | 5.1 |
| Trachea | 0.0 | Melanoma LOX IMVI | 0.8 |
| Kidney | 73.7 | Melanoma* (met) SK-MEL-5 | 0.0 |
| Kidney (fetal) | 8.4 | | |

TABLE 76
Panel 1.3D

| | Relative | | Relative |
|---|---|---|---|
| | Expression (%) | | Expression (%) |
| | 1.3dx4tm5367t_{—} | | 1.3dx4tm5367 |
| Tissue Name | ag1505_b2 | Tissue Name | t_ag1505_b2 |
| Liver adenocarcinoma | 0.0 | Kidney (fetal) | 10.1 |
| Pancreas | 0.0 | Renal ca. 786-0 | 0.0 |
| Pancreatic ca. CAPAN 2 | 0.0 | Renal ca. A498 | 0.0 |
| Adrenal gland | 6.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 0.0 | Liver | 18.3 |
| Brain (whole) | 0.0 | Liver (fetal) | 4.2 |
| Brain (amygdala) | 0.0 | Liver ca. (hepatoblast) HepG2 | 0.0 |
| Brain (cerebellum) | 0.0 | Lung | 0.0 |
| Brain (hippocampus) | 0.0 | Lung (fetal) | 0.0 |
| Brain (substantia nigra) | 0.0 | Lung ca. (small cell) LX-1 | 0.0 |
| Brain (thalamus) | 0.0 | Lung ca. (small cell) NCI-H69 | 0.0 |
| Cerebral Cortex | 0.0 | Lung ca. (s.cell var.) SHP-77 | 0.0 |
| Spinal cord | 0.0 | Lung ca. (large cell) NCI-H460 | 0.0 |
| CNS ca. (glio/astro) U87-MG | 0.0 | Lung ca. (non-sm. cell) A549 | 12.0 |
| CNS ca. (glio/astro) U-118-MG | 0.0 | Lung ca. (non-s.cell) NCI-H23 | 0.0 |
| CNS ca. (astro) SW1783 | 0.0 | Lung ca (non-s.cell) HOP-62 | 0.0 |
| CNS ca.* (neuro; met) SK-N-AS | 5.4 | Lung ca. (non-s.cl) NCI-H522 | 0.0 |
| CNS ca. (astro) SF-539 | 0.0 | Lung ca. (squam.) SW 900 | 0.0 |
| CNS ca. (astro) SNB-75 | 0.0 | Lung ca. (squam.) NCI-H596 | 0.0 |
| CNS ca. (glio) SNB-19 | 0.0 | Mammary gland | 7.4 |
| CNS ca. (glio) U251 | 100.0 | Breast ca.* (pl. effusion) MCF-7 | 0.0 |
| CNS ca. (glio) SF-295 | 0.0 | Breast ca.* (pl.ef) MDA-MB-231 | 0.0 |
| Heart (fetal) | 0.0 | Breast ca.* (pl. effusion) T47D | 0.0 |
| Heart | 0.0 | Breast ca. BT-549 | 0.0 |
| Fetal Skeletal | 13.0 | Breast ca. MDA-N | 0.0 |
| Skeletal muscle | 0.0 | Ovary | 0.0 |
| Bone marrow | 0.0 | Ovarian ca. OVCAR-3 | 0.0 |
| Thymus | 0.0 | Ovarian ca. OVCAR-4 | 0.0 |
| Spleen | 0.0 | Ovarian ca. OVCAR-5 | 0.0 |
| Lymph node | 0.0 | Ovarian ca. OVCAR-8 | 0.0 |
| Colorectal | 0.0 | Ovarian ca. IGROV-1 | 0.0 |
| Stomach | 19.8 | Ovarian ca.* (ascites) SK-OV-3 | 0.0 |
| Small intestine | 2.2 | Uterus | 0.0 |
| Colon ca. SW480 | 0.0 | Placenta | 0.0 |
| Colon ca.* (SW480 met) SW620 | 0.0 | Prostate | 0.0 |
| Colon ca. HT29 | 0.0 | Prostate ca.* (bone met) PC-3 | 0.0 |
| Colon ca. HCT-116 | 0.0 | Testis | 5.7 |
| Colon ca. CaCo-2 | 0.0 | Melanoma Hs688(A).T | 0.0 |
| 83219 CC Well to Mod Diff | 0.0 | Melanoma* (met) Hs688(B).T | 0.0 |
| (ODO3866) | | | |
| Colon ca. HCC-2998 | 0.0 | Melanoma UACC-62 | 0.0 |
| Gastric ca.* (liver met) NCI-N87 | 21.4 | Melanoma M14 | 0.0 |
| Bladder | 0.0 | Melanoma LOX IMVI | 3.6 |
| Trachea | 0.0 | Melanoma* (met) SK-MEL-5 | 0.0 |
| Kidney | 0.0 | Adipose | 2.8 |

TABLE 77
Panel 2.2

| | Relative | | Relative |
|---|---|---|---|
| | Expression (%) | | Expression (%) |
| | 2.2x4tm6351t_{—} | | 2.2x4tm6351 |
| Tissue Name | ag1505_a2 | Tissue Name | t_ag1505_a2 |
| Normal Colon GENPAK 061003 | 0.0 | 83793 Kidney NAT (OD04348) | 12.9 |
| 97759 Colon cancer (OD06064) | 0.0 | 98938 Kidney malignant cancer | 4.1 |
| | | (OD06204B) | |
| 97760 Colon cancer NAT | 0.0 | 98939 Kidney normal adjacent | 7.6 |
| (OD06064) | | tissue (OD06204E) | |
| 97778 Colon cancer (OD06159) | 0.0 | 85973 Kidney Cancer (OD04450- | 0.0 |
| | | 01) | |
| 97779 Colon cancer NAT | 1.7 | 85974 Kidney NAT (OD04450-03) | 2.5 |
| (OD06159) | | | |
| 98861 Colon cancer (OD06297-04) | 0.0 | Kidney Cancer Clontech 8120613 | 0.0 |
| 98862 Colon cancer NAT | 0.0 | Kidney NAT Clontech 8120614 | 11.9 |
| (OD06297-015) | | | |
| 83237 CC Gr.2 ascend colon | 0.0 | Kidney Cancer Clontech 9010320 | 0.0 |
| (ODO3921) | | | |
| 83238 CC NAT (ODO3921) | 0.0 | Kidney NAT Clontech 9010321 | 0.0 |
| 97766 Colon cancer metastasis | 0.0 | Kidney Cancer Clontech 8120607 | 0.0 |
| (OD06104) | | | |
| 97767 Lung NAT (OD06104) | 0.0 | Kidney NAT Clontech 8120608 | 0.0 |
| 87472 Colon mets to lung | 0.0 | Normal Uterus GENPAK 061018 | 0.0 |
| (OD04451-01) | | | |
| 87473 Lung NAT (OD04451-02) | 0.0 | Uterus Cancer GENPAK 064011 | 0.0 |
| Normal Prostate Clontech A+ | 0.0 | Normal Thyroid Clontech A+ | 0.0 |
| 6546-1 (8090438) | | 6570-1 (7080817) | |
| 84140 Prostate Cancer (OD04410) | 0.0 | Thyroid Cancer GENPAK 064010 | 0.0 |
| 84141 Prostate NAT (OD04410) | 0.0 | Thyroid Cancer INVITROGEN | 0.0 |
| | | A302152 | |
| Normal Ovary Res. Gen. | 2.2 | Thyroid NAT INVITROGEN | 0.0 |
| | | A302153 | |
| 98863 Ovarian cancer (OD06283- | 0.0 | Normal Breast GENPAK 061019 | 100.0 |
| 03) | | | |
| 98865 Ovarian cancer | 0.0 | 84877 Breast Cancer (OD04566) | 0.0 |
| NAT/fallopian tube (OD06283-07) | | | |
| Ovarian Cancer GENPAK 064008 | 0.0 | Breast Cancer Res. Gen. 1024 | 4.9 |
| 97773 Ovarian cancer (OD06145) | 3.5 | 85975 Breast Cancer (OD04590- | 0.0 |
| | | 01) | |
| 97775 Ovarian cancer NAT | 0.0 | 85976 Breast Cancer Mets | 0.0 |
| (OD06145) | | (OD04590-03) | |
| 98853 Ovarian cancer (OD06455- | 0.0 | 87070 Breast Cancer Metastasis | 0.0 |
| 03) | | (OD04655-05) | |
| 98854 Ovarian NAT (OD06455- | 0.0 | GENPAK Breast Cancer 064006 | 0.0 |
| 07) Fallopian tube | | | |
| Normal Lung GENPAK 061010 | 0.0 | Breast Cancer Clontech 9100266 | 0.0 |
| 92337 Invasive poor diff. lung | 0.0 | Breast NAT Clontech 9100265 | 0.0 |
| adeno (ODO4945-01 | | | |
| 92338 Lung NAT (ODO4945-03) | 4.0 | Breast Cancer INVITROGEN | 5.9 |
| | | A209073 | |
| 84136 Lung Malignant Cancer | 0.0 | Breast NAT INVITROGEN | 48.3 |
| (OD03126) | | A2090734 | |
| 84137 Lung NAT (OD03126) | 0.0 | 97763 Breast cancer (OD06083) | 14.2 |
| 90372 Lung Cancer (OD05014A) | 2.7 | 97764 Breast cancer node | 0.0 |
| | | metastasis (OD06083) | |
| 90373 Lung NAT (OD05014B) | 0.0 | Normal Liver GENPAK 061009 | 47.4 |
| 97761 Lung cancer (OD06081) | 0.0 | Liver Cancer Research Genetics | 0.0 |
| | | RNA 1026 | |
| 97762 Lung cancer NAT | 0.0 | Liver Cancer Research Genetics | 0.0 |
| (OD06081) | | RNA 1025 | |
| 85950 Lung Cancer (OD04237-01) | 0.0 | Paired Liver Cancer Tissue | 0.0 |
| | | Research Genetics RNA 6004-T | |
| 85970 Lung NAT (OD04237-02) | 0.0 | Paired Liver Tissue Research | 5.5 |
| | | Genetics RNA 6004-N | |
| 83255 Ocular Mel Met to Liver | 0.0 | Paired Liver Cancer Tissue | 0.0 |
| (ODO4310) | | Research Genetics RNA 6005-T | |
| 83256 Liver NAT (ODO4310) | 4.5 | Paired Liver Tissue Research | 0.0 |
| | | Genetics RNA 6005-N | |
| 84139 Melanoma Mets to Lung | 0.0 | Liver Cancer GENPAK 064003 | 1.5 |
| (OD04321) | | | |
| 84138 Lung NAT (OD04321) | 0.0 | Normal Bladder GENPAK 061001 | 0.0 |
| Normal Kidney GENPAK 061008 | 2.7 | Bladder Cancer Research Genetics | 0.0 |
| | | RNA 1023 | |
| 83786 Kidney Ca, Nuclear grade 2 | 27.4 | Bladder Cancer INVITROGEN | 0.0 |
| (OD04338) | | A302173 | |
| 83787 Kidney NAT (OD04338) | 0.0 | Normal Stomach GENPAK | 67.3 |
| | | 061017 | |
| 83788 Kidney Ca Nuclear grade | 0.0 | Gastric Cancer Clontech 9060397 | 0.0 |
| 1/2 (OD04339) | | | |
| 83789 Kidney NAT (OD04339) | 3.5 | NAT Stomach Clontech 9060396 | 4.1 |
| 83790 Kidney Ca, Clear cell type | 0.0 | Gastric Cancer Clontech 9060395 | 0.0 |
| (OD04340) | | | |
| 83791 Kidney NAT (OD04340) | 14.8 | NAT Stomach Clontech 9060394 | 7.7 |
| 83792 Kidney Ca, Nuclear grade 3 | 0.0 | Gastric Cancer GENPAK 064005 | 0.0 |
| (OD04348) | | | |

TABLE 78
Panel 3D

| | Relative | | Relative |
|---|---|---|---|
| | Expression (%) | | Expression (%) |
| | 3dtm4935t_{—} | | 3dtm4935t_ag |
| Tissue Name | ag1505 | Tissue Name | 1505 |
| 94905_Daoy_Medulloblastoma/Ce | 0.9 | 94954_Ca Ski_Cervical | 0.0 |
| rebellum_sscDNA | | epidermoid carcinoma | |
| | | (metastasis)_sscDNA | |
| 94906_TE671_Medulloblastom/Ce | 0.5 | 94955_ES-2_Ovarian clear cell | 0.7 |
| rebellum_sscDNA | | carcinoma_sscDNA | |
| 94907_D283 | 9.3 | 94957_Ramos/6h stim_Stimulated | 0.0 |
| Med_Medulloblastoma/ | | with PMA/ionomycin 6h_sscDNA | |
| Cerebellum_sscDNA | | | |
| 94908_PFSK-1_Primitive | 3.3 | 94958_Ramos/14h stim_{—} | 0.0 |
| Neuroectodermal/Cerebellum_ssc | | Stimulated with PMA/ionomycin | |
| DNA | | 14h_sscDNA | |
| 94909_XF-498_CNS_sscDNA | 0.8 | 94962_MEG-01_Chronic | 0.0 |
| | | myelogenous leukemia | |
| | | (megokaryoblast)_sscDNA | |
| 94910_SNB- | 1.4 | 94963_Raji_Burkitt's | 0.0 |
| 78_CNS/glioma_sscDNA | | lymphoma_sscDNA | |
| 94911_SF- | 3.2 | 94964_Daudi_Burkitt's | 0.0 |
| 268_CNS/glioblastoma_sscDNA | | lymphoma_sscDNA | |
| 94912_T98G_Glioblastoma_sscD | 0.7 | 94965_U266_B-cell | 0.0 |
| NA | | plasmacytoma/myeloma_sscDNA | |
| 96776_SK-N-SH_Neuroblastoma | 0.7 | 94968_CA46_Burkitt's | 0.0 |
| (metastasis)_sscDNA | | lymphoma_sscDNA | |
| 94913_SF- | 0.0 | 94970_RL_non-Hodgkin's B-cell | 0.0 |
| 295_CNS/glioblastoma_sscDNA | | lymphoma_sscDNA | |
| 94914_Cerebellum_sscDNA | 0.0 | 94972_JM1_pre-B-cell | 0.0 |
| | | lymphoma/leukemia_sscDNA | |
| 96777_Cerebellum_sscDNA | 0.0 | 94973_Jurkat_T cell | 0.0 |
| | | leukemia_sscDNA | |
| 94916_NCI- | 0.3 | 94974_TF- | 0.0 |
| H292_Mucoepidermoid lung | | 1_Erythroleukemia_sscDNA | |
| carcinoma_sscDNA | | | |
| 94917_DMS-114_Small cell lung | 0.0 | 94975_HUT 78_T-cell | 0.0 |
| cancer_sscDNA | | lymphoma_sscDNA | |
| 94918_DMS-79_Small cell lung | 100.0 | 94977_U937_Histiocytic | 0.0 |
| cancer/neuroendocrine_sscDNA | | lymphoma_sscDNA | |
| 94919_NCI-H146_Small cell lung | 1.9 | 94980_KU-812_Myelogenous | 0.0 |
| cancer/neuroendocrine_sscDNA | | leukemia_sscDNA | |
| 94920_NCI-H526_Small cell lung | 0.1 | 94981_769-P_Clear cell renal | 0.0 |
| cancer/neuroendocrine_sscDNA | | carcinoma_sscDNA | |
| 94921_NCI-N417_Small cell lung | 1.5 | 94983_Caki-2_Clear cell renal | 0.0 |
| cancer/neuroendocrine_sscDNA | | carcinoma_sscDNA | |
| 94923_NCI-H82_Small cell lung | 0.0 | 94984_SW 839_Clear cell renal | 0.0 |
| cancer/neuroendocrine_sscDNA | | carcinoma_sscDNA | |
| 94924_NCI-H157_Squamous cell | 0.0 | 94986_G401_Wilms' | 0.0 |
| lung cancer (metastasis)_sscDNA | | tumor_sscDNA | |
| 94925_NCI-H1155_Large cell | 0.0 | 94987_Hs766T_Pancreatic | 1.4 |
| lung | | carcinoma (LN | |
| cancer/neuroendocrine_sscDNA | | metastasis)_sscDNA | |
| 94926_NCI-H1299_Large cell | 0.0 | 94988_CAPAN-1_Pancreatic | 0.0 |
| lung | | adenocarcinoma (liver | |
| cancer/neuroendocrine_sscDNA | | metastasis)_sscDNA | |
| 94927_NCI-H727_Lung | 0.0 | 94989_SU86.86_Pancreatic | 0.7 |
| carcinoid_sscDNA | | carcinoma (liver | |
| | | metastasis)_sscDNA | |
| 94928_NCI-UMC-11_Lung | 16.3 | 94990_BxPC-3_Pancreatic | 0.5 |
| carcinoid_sscDNA | | adenocarcinoma_sscDNA | |
| 94929_LX-1_Small cell lung | 0.4 | 94991_HPAC_Pancreatic | 2.2 |
| cancer_sscDNA | | adenocarcinoma_sscDNA | |
| 94930_Colo-205_Colon | 0.0 | 94992_MIA PaCa-2_Pancreatic | 0.0 |
| cancer_sscDNA | | carcinoma_sscDNA | |
| 94931_KM12_Colon | 0.0 | 94993_CFPAC-1_Pancreatic | 0.0 |
| cancer_sscDNA | | ductal adenocarcinoma_sscDNA | |
| 94932_KM20L2_Colon | 0.0 | 94994_PANC-1_Pancreatic | 0.0 |
| cancer_sscDNA | | epithelioid ductal | |
| | | carcinoma_sscDNA | |
| 94933_NCI-H716_Colon | 0.0 | 94996_T24_Bladder carcinma | 0.0 |
| cancer_sscDNA | | (transitional cell)_sscDNA | |
| 94935_SW-48_Colon | 0.0 | 94997_5637_Bladder | 0.0 |
| adenocarcinoma_sscDNA | | carcinoma_sscDNA | |
| 94936_SW1116_Colon | 0.0 | 94998_HT-1197_Bladder | 1.7 |
| adenocarcinoma_sscDNA | | carcinoma_sscDNA | |
| 94937_LS 174T_Colon | 0.0 | 94999_UM-UC-3_Bladder | 0.2 |
| adenocarcinoma_sscDNA | | carcinma (transitional | |
| | | cell)_sscDNA | |
| 94938_SW-948_Colon | 0.0 | 95000_A204_Rhabdomyosarcoma_{—} | 0.1 |
| adenocarcinoma_sscDNA | | sscDNA | |
| 94939_SW-480_Colon | 0.0 | 95001_HT- | 0.1 |
| adenocarcinoma_sscDNA | | 1080_Fibrosarcoma_sscDNA | |
| 94940_NCI-SNU-5_Gastric | 0.0 | 95002_MG-63_Osteosarcoma | 0.0 |
| carcinoma_sscDNA | | (bone)_sscDNA | |
| 94941_KATO III_Gastric | 0.0 | 95003_SK-LMS- | 8.8 |
| carcinoma_sscDNA | | 1_Leiomyosarcoma | |
| | | (vulva)_sscDNA | |
| 94943_NCI-SNU-16_Gastric | 5.8 | 95004_SJRH30_Rhabdomyosarcoma | 0.0 |
| carcinoma_sscDNA | | (met to bone marrow)_sscDNA | |
| 94944_NCI-SNU-1_Gastric | 0.0 | 95005_A431_Epidermoid | 0.0 |
| carcinoma_sscDNA | | carcinoma_sscDNA | |
| 94946_RF-1_Gastric | 0.0 | 95007_WM266- | 0.0 |
| adenocarcinoma_sscDNA | | 4_Melanoma_sscDNA | |
| 94947_RF-48_Gastric | 0.0 | 95010_DU 145_Prostate | 0.0 |
| adenocarcinoma_sscDNA | | carcinoma (brain | |
| | | metastasis)_sscDNA | |
| 96778_MKN-45_Gastric | 0.0 | 95012_MDA-MB-468_Breast | 0.0 |
| carcinoma_sscDNA | | adenocarcinoma_sscDNA | |
| 94949_NCI-N87_Gastric | 1.0 | 95013_SCC-4_Squamous cell | 0.0 |
| carcinoma_sscDNA | | carcinoma of tongue_sscDNA | |
| 94951_OVCAR-5_Ovarian | 0.0 | 95014_SCC-9_Squamous cell | 0.0 |
| carcinoma_sscDNA | | carcinoma of tongue_sscDNA | |
| 94952_RL95-2_Uterine | 0.0 | 95015_SCC-15_Squamous cell | 0.1 |
| carcinoma_sscDNA | | carcinoma of tongue_sscDNA | |
| 94953_HelaS3_Cervical | 0.0 | 95017_CAL 27_Squamous cell | 10.7 |
| adenocarcinoma_sscDNA | | carcinoma of tongue_sscDNA | |

Panel 1.2 Summary Ag1505 The expression of this gene in panel 1.2 appears to be highest in a sample derived from a gastric cancer cell line (NCI-N87) (CT=30.4). Interestingly, this gene is more highly expressed in adult kidney tissue (CT=30.6) than in fetal kidney. Expression of the NOV12 gene is also detected in the hippocampus (CT=33.3) and in two CNS cancer cell lines (CTs=33.2, 34.5). Thus, the expression of the NOV12 gene could be used to distinguish gastric cancer from other tissues or to distinguish adult kidney tissue from fetal kidney tissue. Moreover, therapeutic modulation of the expression or activity of the NOV12 gene product, through the use of small molecule drugs or antibodies, might be of benefit in the treatment of gastric cancer.

Among tissues involved in metabolic processes, the NOV12 gene is expressed at significant levels in both adult and fetal liver (adult CT=32.5, fetal CT=32.8) and may play a role as a small molecule target in the treatment of any or all diseases of the liver.

For tissues involved in the central nervous system, the NOV12 gene is homologous to human S4 protein, a proteasome complex complex subunit, which interacts with hepatitis B virus (HBV) X-protein (HBX). A peptide derived from the S4 protein may be used to interfere with HBV infection, and is thus useful in therapy of hepatitis B. Such peptides are also useful as antigens to generate polyclonal or monoclonal antibodies for diagnostic applications. DNA probes and primers derived from the NOV12 gene may also be used to detect HBV infection. The proteasome mediates the degradation of ubiquitinated intracellular proteins. Numerous neurodegenerative diseases have been associated with improper ubiquitination and targeting of proteins to the proteasome. For example, alpha synuclein, which mediates Parkinson's disease, associates with a subunit of the regulatory complex of the proteasome, suggesting that the mutated alpha synuclein changes proteasomal activity and results in the disease. Parkin has ubiquitin ligase activity disrupted by mutations that induce early onset Parkinson's disease. Alzheimer's disease is also associated with improper ubiquitination and subsequent degradation of proteins by the proteasome. Phosphorylation of the S4 protein in response to gamma interferon decreases the level of the protein and thus regulates its function. Thus, agents that affect the phosphorylation and level of the NOV12 gene product may be useful in influencing proteasome activity and consequently abberant neurodegenerative protein degradation involved in Parkinson's disease, Alzheimer's disease, and other neurodegenerative disorders. Such agents would be useful in treatment of these diseases.

Panel 1.3D Summary Ag1505 Low levels of NOV12 gene expression are detected in a CNS cancer cell line (CT=34).

Panel 2.2 Summary Ag1505 Expression of the NOV12 gene in this panel is detected only in normal tissues. In all three tissue types where the gene is detected, the NOV12 gene is overexpressed in normal tissue when compared to corresponding cancerous tissue. The NOV12 gene is expressed in normal breast (CT=33.4), normal liver (CT=34.5) and stomach (CT=34), and undetected in the corresponding cancerous tissues. Thus, the expression of this gene could be used to distinguish normal breast, stomach and liver tissues from other tissues.

Panel 3D Summary Ag1505 High expression of the NOV12 gene is detected in a small cell lung cancer line (CT=28.6). Moderate levels of expression are detected in carcinoma of the tongue (CT=31.9) and low levels of gene expression are detected in bladder, gastric, pancreatic cancers and leiomyosarcoma. Thus, the expression of the NOV12 gene could be used to distinguish these tissues from other samples. In addition, therapeutic modulation of the expression or activity of the NOV12 gene or its protein product, through the use of small molecule drugs or antibodies, might be of benefit in the treatment of small cell lung cancer. (Layfield et al., Neuropathol Appl Neurobiol. 27:171-9, 2001; Ghee et al., J Neurochem.75: 2221-4, 2000; Rivett et al., Biochimie 83:363-6, 2001.)

### Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. An isolated polypeptide comprising an amino acid sequence selected from the group consisting of:
(a) a mature form of an amino acid sequence selected from the group consisting of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64 and 200;
(b) a variant of a mature form of an amino acid sequence selected from the group consisting of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64 and 200, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64 and 200; and
(d) a variant of an amino acid sequence selected from the group consisting of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64 and 200 wherein one or more amino acid residues in said variant differs from the amino acid sequence of said mature form, provided that said variant differs in no more than 15% of amino acid residues from said amino acid sequence.

2. The polypeptide of claim 1, wherein said polypeptide comprises the amino acid sequence of a naturally-occurring allelic variant of an amino acid sequence selected from the group consisting of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64 and 200.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64 and 200;
(b) a variant of a mature form of an amino acid sequence selected from the group consisting of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64 and 200, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64 and 200;
(d) a variant of an amino acid sequence selected from the group consisting of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64 and 200, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64 and 200, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199.

9. The nucleic acid molecule of claim 5, wherein said nucleic acid molecule comprises a nucleotide sequence selected from the group consisting of
(a) a nucleotide sequence selected from the group consisting of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199;
(b) a nucleotide sequence differing by one or more nucleotides from a nucleotide sequence selected from the group consisting of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63 and 199, or a complement of said nucleotide sequence.

11. The nucleic acid molecule of claim 5, wherein the nucleic acid molecule comprises a nucleotide sequence selected from the group consisting of
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that immunospecifically-binds to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide,
thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule,
thereby determining the presence or amount of the nucleic acid molecule in said sample.

20. A method of identifying an agent that binds to a polypeptide of claim 1, the method comprising:
(a) contacting said polypeptide with said agent; and
(b) determining whether said agent binds to said polypeptide.

21. A method for identifying an agent that modulates the expression or activity of the polypeptide of claim 1, the method comprising:
(a) providing a cell expressing said polypeptide;
(b) contacting the cell with said agent; and
(c) determining whether the agent modulates expression or activity of said polypeptide,
whereby an alteration in expression or activity of said peptide indicates said agent modulates expression or activity of said polypeptide.

22. A method for modulating the activity of the polypeptide of claim 1, the method comprising contacting a cell sample expressing the polypeptide of said claim with a compound that binds to said polypeptide in an amount sufficient to modulate the activity of the polypeptide.

23. A method of treating or preventing a NOVX-associated disorder, said method comprising administering to a subject in which such treatment or prevention is desired the polypeptide of claim 1 in an amount sufficient to treat or prevent said NOVX-associated disorder in said subject.

24. The method of claim 23, wherein said subject is a human.

25. A method of treating or preventing a NOVX-associated disorder, said method comprising administering to a subject in which such treatment or prevention is desired the nucleic acid of claim 5 in an amount sufficient to treat or prevent said NOVX-associated disorder in said subject.

26. The method of claim 25, wherein said subject is a human.

27. A method of treating or preventing a NOVX-associated disorder, said method comprising administering to a subject in which such treatment or prevention is desired the antibody of claim 15 in an amount sufficient to treat or prevent said NOVX-associated disorder in said subject.

28. The method of claim 27, wherein the subject is a human.

29. A pharmaceutical composition comprising the polypeptide of claim 1 and a pharmaceutically-acceptable carrier.

30. A pharmaceutical composition comprising the nucleic acid molecule of claim 5 and a pharmaceutically-acceptable carrier.

31. A pharmaceutical composition comprising the antibody of claim 15 and a pharmaceutically-acceptable carrier.

32. A kit comprising in one or more containers, the pharmaceutical composition of claim 29.

33. A kit comprising in one or more containers, the pharmaceutical composition of claim 30.

34. A kit comprising in one or more containers, the pharmaceutical composition of claim 31.

35. The use of a therapeutic in the manufacture of a medicament for treating a syndrome associated with a human disease, the disease selected from a NOVX-associated disorder, wherein said therapeutic is selected from the group consisting of a NOVX polypeptide, a NOVX nucleic acid, and a NOVX antibody.

36. A method for screening for a modulator of activity or of latency or predisposition to a NOVX-associated disorder, said method comprising:
(a) administering a test compound to a test animal at increased risk for a NOVX-associated disorder, wherein said test animal recombinantly expresses the polypeptide of claim 1;
(b) measuring the activity of said polypeptide in said test animal after administering the compound of step (a);
(c) comparing the activity of said protein in said test animal with the activity of said polypeptide in a control animal not administered said polypeptide, wherein a change in the activity of said polypeptide in said test animal relative to said control animal indicates the test compound is a modulator of latency of or predisposition to a NOVX-associated disorder.

37. The method of claim 36, wherein said test animal is a recombinant test animal that expresses a test protein transgene or expresses said transgene under the control of a promoter at an increased level relative to a wild-type test animal, and wherein said promoter is not the native gene promoter of said transgene.

38. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease,
wherein an alteration in the expression level of the polypeptide in the first subject as compared to the control sample indicates the presence of or predisposition to said disease.

39. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

40. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64 and 200, or a biologically active fragment thereof.

41. A method of treating a pathological state in a mammal, the method comprising administering to the mammal the antibody of claim 15 in an amount sufficient to alleviate the pathological state.
